(19) United States
(12) Patent Application Publication
(10) Pub. No.: US 2006/0141493 A1
(43) Pub. Date: Jun. 29, 2006

(54) Atherosclerotic phenotype determinative genes and methods for using the same

(75) Inventors: Mike West, Durham, NC (US); Joseph R. Nevins, Chapel Hill, NC (US); Pascal Goldschmidt, Chapel Hill, NC (US); David Seo, Durham, NC (US)

Correspondence Address: FISH & NEAVE IP GROUP;ROPES & GRAY LLP, ONE INTERNATIONAL PLACE, BOSTON, MA, 02110-2624, US

(73) Assignee: Duke University Office of Science and Technology, Durham, NC (US)

(21) Appl. No.: 11/198,782

(22) Filed: Aug. 4, 2005

Related U.S. Application Data
(63) Continuation-in-part of application No. 10/291,885, filed on Nov. 12, 2002.
(60) Provisional application No. 60/337,709, filed on Nov. 9, 2001.
(60) Provisional application No. 60/374,547, filed on Apr. 23, 2002.
(60) Provisional application No. 60/420,784, filed on Oct. 24, 2002.
(60) Provisional application No. 60/421,043, filed on Oct. 25, 2002.
(60) Provisional application No. 60/424,680, filed on Nov. 8, 2002.
(60) Provisional application No. 60/651,462, filed on Aug. 4, 2004.

(51) Int. Cl. C12Q 1/68; G06F 19/00

(52) U.S. Cl. 435/6; 702/20

(57) ABSTRACT

Genes whose expression is correlated with and determinant of an atherosclerotic phenotype are provided. Genes whose expression is correlated with and determinant of an atherosclerotic susceptibility are also provided. Also provided are methods of using the subject atherosclerotic determinant genes or the atherosclerotic susceptibility genes in diagnosis and treatment methods, as well as drug screening methods. In addition, reagents and kits thereof that find use in practicing the subject methods are provided. Also provided are methods of determining whether a gene is correlated with a disease phenotype, where correlation is determined using at least one parameter that is not expression level and is preferably determined using a binary prediction tree analysis.

### CROSS-REFERENCE TO RELATED APPLICATIONS

This application is a continuation-in-part application of prior U.S. application Ser. No. 10/291,885, filed Nov. 12, 2002, entitled “ATHEROSCLEROTIC PHENOTYPE DETERMINATIVE GENES AND METHODS FOR USING THE SAME,” which claims the benefit of U.S. Provisional Application No. 60/337,709 filed Nov. 9, 2001, 60/374,547 filed Apr. 23, 2002, 60/420,784 filed Oct. 24, 2002, 60/421,043 filed Oct. 25, 2002 and 60/424,680 filed Nov. 8, 2002. This application also claims priority under 35 USC 119(e) to U.S. Provisional Application No. 60/651,462, filed Aug. 4, 2004. The entire teachings of all these patent applications are incorporated herein by reference.

### STATEMENT REGARDING FEDERALLY-SPONSORED RESEARCH OR DEVELOPMENT

The invention described herein was supported, in whole or in part, by Grant No. HL073042-02 from the National Heart, Lung and Blood Institute at the National Institutes of Health. The United States government has certain rights in the invention.

### FIELD OF THE INVENTION

The field of this invention is atherosclerosis.

### BACKGROUND OF THE INVENTION

Atherosclerosis is the leading cause of morbidity and mortality in the industrialized world. While applicants have made substantial progress in the treatment and prevention of atherosclerosis and the related thromboembolic complications, there remains an urgent need to develop individualized prognostic tools and therapeutic plans. Progress in this direction will come with an improved understanding of the genetic foundations of the disease.

Atherosclerosis is a complex trait manifested by chronic inflammation that selectively affects arterial vessels and progressively destroys the structure of the vessel wall, leading to thromboembolic complications. The thromboembolic consequences of atherosclerosis, sudden cardiac death, myocardial infarction, and other ischemic organ damage such as stroke and ischemic renovascular disease, represent the major causes of death, morbidity and disability for developed countries and are spreading rapidly worldwide. In spite of substantial improvement in our understanding of risk for atherosclerosis and thromboembolic complications, improved predictive tools are needed to allow for early prevention in a fashion that is cost-effective.

The sequencing of the entire human genome promises to transform the study of human health by providing an opportunity to develop genomic knowledge that will eventually boost prevention, diagnosis and treatment of disease. Genome research in the post-sequencing era is now faced with massive, multi-disciplinary challenges to realize this promise. Most complex illnesses result (i) from the combined action of gene variants that are considered as “normal”, as they do not destroy the function of the gene that they modify; (ii) from factors provided by the environment, and (iii) from a stochastic component that can be best defined as “chance”. The ensemble of genetic modifiers that enhance the impact of environmental factors on health represents the genetic susceptibility to ailments.

Because of the potential benefits of genetic approaches to diagnosis and treatment of atherosclerosis, there is intense interest in the identification of genes whose contribution is relevant to atherosclerosis. Ideally, one would like to test all variants of all genes for their contribution to atherosclerosis. However, such effort would be prohibitively expensive, and even if the resources were to become accessible, our current ability to analyze data would become limiting. Hence, the prioritization of contributory genes has become a necessity. A systematic approach to satisfy this need and provide such prioritization process has been defined and is based on gene expression that correlates with atherosclerosis. Hence, the present invention satisfies this need.

As with most complex illnesses, atherosclerosis results from the combined interaction of a genetic component and environmental factors. However, unlike classical Mendelian disorders, the genetic component is not attributable to single causative genes making it difficult to study by standard genetic and molecular biological approaches. Instead, it is anticipated that combinations of gene variants determine an individual's susceptibility to atherosclerosis by enhancing the impact of environmental factors.

The gene variants are often in the form of single nucleotide polymorphisms (SNPs). SNPs represent subtle variations in a gene's coding sequence or the associated regulatory regions resulting in a mild to moderate impact on the function or concentration of the encoded protein. The inheritance of unique combinations of genetic variants can have a dominant impact that fosters the pathogenesis of atherosclerosis. In principle, one would like to identify all variants of all genes and assay them for their contribution towards the genesis of atherosclerosis. Even if applicants were able to identify all variants, applicants would be limited by our ability to assay and analyze such a vast number of SNPs. Practically, one must take an approach that falls somewhere between an analysis restricted to known candidate genes identified on the basis of clinical and biological knowledge (functional candidate genes) and an investigation of the entire genomic complement of genes. See Nussbaum R L Genetics in Medicine. New York: W.B. Saunders Company, 2001. Science 1996; 272:689-93.

Such an approach should involve prioritization based on programmatic qualification mechanisms.

Recent advances in the knowledge of the human genome, coupled with the development of technologies for large scale analysis of gene activity via DNA microarrays, now affords the opportunity to identify genes whose expression implies a role in a phenotype.

Therefore novel strategies for analyzing gene expression data to advance the understanding of the disease in relationship to gene expression, are needed to take advantage of the recent development of technologies. Further, new methods to predict and treat atherosclerosis based on the gene expression data obtained by such techniques are needed.

### SUMMARY OF THE INVENTION

The invention relates in part, to methods of diagnosing, or aiding in the diagnosis, of atherosclerosis. The invention also relates to determining the susceptibility, or aiding in determining the susceptibility, of developing atherosclerosis, such as in a mammal. Applicants have used a unique collection of human aorta samples, which exhibit a progression of atherosclerotic disease, coupled with novel strategies for analyzing gene expression data, to identify genes and metagenes whose expression closely relates to, and indeed predicts, the extent of fatty streaks and more advanced atherosclerotic lesions. Applicants believe this represents a novel approach to the identification of genes that contribute to atherosclerosis. Applicants have also analyzed gene expression data from different sections of aorta to identify genes and metagenes indicative of the susceptibility of vascular tissue to becoming atherosclerotic, or of the mammal from which the vascular sample was derived of developing atherosclerosis.

Genes whose expression is correlated with and determinant of an atherosclerotic phenotype are provided. Similarly, metagenes useful in binary prediction tree modeling to classify samples according to atherosclerotic phenotype are also provided. Genes whose expression is correlated with and determinant of susceptibility to atherosclerosis are provided. Similarly, metagenes useful in binary prediction tree modeling to classify samples according to their susceptibility to atherosclerosis are also provided.

The invention also provides methods of using the subject atherosclerotic determinant genes and metagenes in diagnosis and treatment methods, as well as in drug screening methods. In addition, reagents and kits thereof that find use in practicing the subject methods are provided. Also provided are methods of determining whether a gene is correlated with a disease phenotype, e.g., atherosclerosis, where correlation is determined using at least one parameter that is not expression level and is preferably determined using binary prediction tree analysis and metagene construction.

The invention also provides metagenes for atherosclerosis identified by the use of a binary prediction tree model, that characterize multiple patterns of expression of the genes across the samples.

One aspect of the invention provides a method of estimating whether a sample is from tissue having an atherosclerotic phenotype, said method comprising (a) obtaining an expression profile for said sample from at least two of said genes listed in Table I; (b) providing one or more predictive statistical tree models, each model including one or more nodes, each node representing a metagene, each node including a statistical predictive probability of the having an atherosclerotic phenotype, each metagene representing a dominant factor from a group of genes associated with having an atherosclerotic phenotype, wherein at least two genes in the group of genes are selected from those listed in Table I; and (c) determining an estimate of the sample having the atherosclerotic phenotype by averaging the predictions of one or more of the tree models applied to the expression profile of the sample. Steps (a) and (b) may be performed in any order.

In some embodiments, at least two of the genes are selected from those having Genbank accession numbers selected from Y09445, AF053233, U43185, AL050008, AB022718, L10333, M80634, AF044896, X78565, AB011143, X69819, J02947, U78095, D67029, AF013249, AB014574, L13939, L06797, D89077, Y08374, X02317, AB002365, AF084481, D34625, AB011103, AF041259, J05037, AF056087, U81800, AL050262, AB018271, J03011, D12485, U88629, U75308, J03600, AF004709, AB002361, X90858, Z29067, U00952, M80254, AF030339, AJ007395, AF013570, Z22555, L22524, Y07512, Y00093, AB007889, Y08136, L10678, Z98046, D79994, D87074, X81109, AL049946, U78556, M63603, X12451, U89606, AB029018, AF095791, X74039, X90976, U00802, X96752, Z49107, AL080235, AF051851, AF062075, AB000220, AB015718, X78817, AJ000534, M63835, M16336, U32324, M22324, X54162, U57911, M64571, AC005546, AC005546, Y13622, L76191, U60060, AJ011497, D64142, D26350, X15414, D87434, X79204, AB014513, U63127, S59184, X53587, Z84718, AF030409, J04621, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, X97074, D89016, AF022797, M33552, U09578, M21186, M64925, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663,

M63193, D84110, AJ006973, AB002318, U51333, U09577 and U00672. In some embodiments, at least two genes are preferred genes from table I. Preferred genes from table II are genes having Genbank accession numbers selected from Y09445, AF053233, U43185, AL050008, AB022718, L10333, M80634, AF044896, X78565, AB011143, X69819, J02947, U78095, D67029, AF013249, AB014574, L13939, L06797, D89077, Y08374, X02317, AB002365, AF084481, D34625, AB011103, AF041259, J05037, AF056087, U81800, AL050262, AB018271, J03011, D12485, U88629, U75308, J03600, AF004709, AB002361, X90858, Z29067, U00952, M80254, AF030339, AJ007395, AF013570, Z22555, L22524, Y07512, Y00093, AB007889, Y08136, L10678, Z98046, D79994, D87074, X81109, AL049946, U78556, M63603, X12451, U89606, AB029018, AF095791, X74039, X90976, U00802, X96752, Z49107, AL080235, AF051851, AF062075, AB000220, AB015718, X78817, AJ000534, M63835, M16336, U32324, M22324, X54162, U57911, M64571, AC005546, AC005546, Y13622, L76191, U60060, AJ011497, D64142, D26350, X15414, D87434, X79204, AB014513, U63127, S59184, X53587, Z84718, AF030409, J04621, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, X97074, D89016, AF022797, M33552, U09578, M21186, M64925, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663, M63193, D84110, AJ006973, AB002318, U51333, U09577 and U00672.

In some embodiments of the methods for estimating whether a sample is from tissue having an atherosclerotic phenotype, the tissue is a vascular tissue, such as aortic tissue. The tissue is preferably mammalian tissue, such as human, primate or rodent tissue. In some embodiments, the sample is from a mammal suspected of having tissue having an atherosclerotic phenotype or from a mammal is at risk of being afflicted with atherosclerosis. Mammals at risk for being afflicted with atherosclerosis include those having traditional cardiovascular risk factors. Cardiovascular risk factors include but are not limited to cholesterol, HDL cholesterol, systolic blood pressure, cigarette smoking, exercise, alcohol, race, family history of premature coronary artery disease, and medication use, including aspirin, statins, B-blockers and hormone replacement therapy in women. In some embodiments, the methods for estimating whether a sample is from tissue having an atherosclerotic phenotype are carried out in the context of determining if an agent has anti-atherosclerosis properties.

For example, a mammal may be treated with a compound, and a sample is obtained from the mammal to determine if the compound can decrease an atherosclerosis phenotype. In some embodiments, the mammal is a rodent model of atherosclerosis, such as apolipoprotein E (apoE)-deficient C57BL/6 mice. In other embodiments, other mouse disease models are used, such as KK/Ay mice, an animal model of type II diabetes.

In some embodiments of the methods for estimating whether a sample is from tissue having an atherosclerotic phenotype, at least one metagene in at least one of the predictive statistical tree models is one of the 509 metagenes provided herein. In one embodiment, at least one of the metagenes (i) is one of the 509 metagenes and (ii) represents a dominant factor from at least 2, 3, 4, 5, 6, 7, 8, 9, 10, 11, 12, 13, 14, 15, 16, 17, 18, 19 or 20 genes associated with having an atherosclerotic phenotype. In another embodiment, at least one of the statistical tree models has two or more metagenes.

In some embodiments of the methods for estimating whether a sample is from tissue having an atherosclerotic phenotype, one or more predictive statistical tree models correctly classify samples with greater than 85%, 90%, 95%, 98% or 99% accuracy. In other embodiments, the one or more predictive statistical tree models correctly classify samples with an accuracy of up to 90%, 91%, 92%, 93%, 94%, 95%, 96%, 97%, 98% or 99%.

The invention also provides a method of predicting the susceptibility of a mammal for developing atherosclerosis, the method comprising: (a) obtaining an expression profile of at least two of said genes listed in Table II from a sample from the mammal; (b) providing one or more predictive statistical tree models, each model including one or more nodes, each node representing a metagene, each node including a statistical predictive probability of being susceptible to developing atherosclerosis, each metagene representing a dominant factor from a group of genes associated with susceptible to developing atherosclerosis, wherein at least two genes in the group of genes are selected from those listed in Table II; and (c) determining an estimate of the sample being susceptible to developing atherosclerosis by averaging the predictions of one or more of the tree models applied to the expression profile of the sample. Steps (a) and (b) may be performed in any order.

In some embodiments, the at least 2, 3, 4, 5, 6 or 7 of the genes are selected from genes having Genbank accession numbers selected from M68891, X51757, D83004, X06256, Z22865, X75918 and M55153.

In some embodiments of the methods for predicting the susceptibility of a mammal for developing atherosclerosis, the tissue is a vascular tissue, such as aortic tissue. The tissue is preferably mammalian tissue, such as human, primate or rodent tissue. In some embodiments, the sample is from a mammal suspected of having tissue susceptible to developing atherosclerosis. Susceptible mammals include those having traditional cardiovascular risk factors.

In some embodiments, the methods for predicting the susceptibility of a mammal for developing atherosclerosis are carried out in the context of determining if an agent can modify the susceptibility of a mammal for developing atherosclerosis. For example, a mammal may be treated with a compound, and a sample is obtained from the mammal to determine if the compound can decrease the susceptibility of a mammal for developing atherosclerosis. In some embodiments, the mammal is a rodent, such as a rodent model of atherosclerosis. Mice atherosclerosis models include as apolipoprotein E (apoE)-deficient C57BL/6 mice. In other embodiments, other mouse disease models are used, such as KK/Ay mice, an animal model of type II diabetes.

In some embodiments of the methods for predicting the susceptibility of a mammal for developing atherosclerosis, at least one metagene in at least one of the predictive statistical tree models is one of the 509 metagenes provided herein. In one embodiment, at least one of the metagenes (i) is one of the 509 metagenes and (ii) represents a dominant factor from at least 2, 3, 4, 5, 6, 7, 8, 9, 10, 11, 12, 13, 14, 15, 16, 17, 18, 19 or 20 genes associated with having an atherosclerotic phenotype. In another embodiment, at least one of the statistical tree models has two or more metagenes.

In some embodiments of the methods for predicting the susceptibility of a mammal for developing atherosclerosis, one or more predictive statistical tree models correctly classify samples with greater than 85%, 90%, 95%, 98% or 99% accuracy. In other embodiments, the one or more predictive statistical tree models correctly classify samples with an accuracy of up to 90%, 91%, 92%, 93%, 94% or 95%.

### BRIEF DESCRIPTION OF THE FIGURES

FIG. 1 shows an example prediction tree for cookie fat outcomes. The root node splits on predictor/factor 92, followed by two subsequent splits on additional predictors 330 and 305. The Π values are point estimates of the predictive probabilities of high fat versus low fat at each of the nodes, with suffixes simply indexing nodes. The labels Z(0=1) indicate the numbers of low fat (0) and high fat (1) samples within each node, and the F# symbols indicate the thresholds that define the predictor based splits within each node.

FIG. 2 shows two predictive factors in cookie dough analysis. All samples are represented by index numbers 1 through 78. Training data are denoted by blue (low fat) and red (high fat), and validation data by cyan (low fat) and magenta (high fat). The two full lines (black) demark the thresholds on the two predictors in this example tree.

FIG. 3 shows a scatter plot of cookie data on three factors in example tree. Samples are denoted by blue (low fat) and red (high fat), with training data represented by filled circles and validation data by open circles.

FIG. 4 shows three ER related metagenes in 49 primary breast tumors. Samples are denoted by blue (ER negative) and red (ER positive), with training data represented by filled circles and validation data by open circles.

FIG. 5 shows three ER related metagenes in 49 primary breast tumors. All samples are represented by index number in 1-78. Training data are denoted by blue (ER negative) and red (ER positive), and validation data by cyan (ER negative) and magenta (ER positive).

FIG. 6 shows honest predictions of ER status of breast tumors. Predictive probabilities are indicated, for each tumor, by the index number on the vertical probability scale, together with an approximate 90% uncertainty interval about the estimated probability. All probabilities are referenced to a notional initial probability (incidence rate) of 0.5 for comparison. Training data are denoted by blue (ER negative) and red (ER positive), and validation data by cyan (ER negative) and magenta (ER positive).

FIG. 7 shows cross-validation probability predictions of lymph node status. Samples (tumors) are plotted by index number, and the plotted numbers are marked on the vertical scale at the estimated predictive probabilities of high risk (red) versus low risk (blue). Approximate 90% uncertainty (confidence) intervals about these estimated probabilities are indicated by vertical dashed lines.

FIG. 8 shows gene expression patterns from the major metagene that predicts lymph node status. Samples are plotted by sample index number and by color (color coding as in FIG. 7).

FIG. 9 shows cross-validation probability predictions of 3-year recurrence. Samples (tumors) are plotted by index number, and the plotted numbers are marked on the vertical scale at the estimated predictive probabilities of 3 year recurrence (red) versus 3 year recurrence free survival (blue). Approximate 90% uncertainty intervals about these estimated probabilities are indicated by vertical dashed lines.

FIG. 10 shows a diagram of aorta processing. The thoracic aorta is harvested, divided along the ventral aspect and furthered sectioned as shown. The A and B strips are used for RNA extraction. The C strip is evaluated for Sudanophilia and raised lesion mapping.

FIG. 11 shows a summary of results of cross validation analysis of disease burden analysis. Samples are plotted by the probability that they are severely diseased (95% CI). Severely diseased samples are red and minimally diseased, blue.

FIG. 12 shows a summary of results of cross validation analysis of aorta location. Samples are plotted by the probability that they are from the distal location in the aorta (95% CI). Distal sections are red and proximal sections, blue.

FIG. 13 shows a graphical display of gene expression in the key metagene for aortic location. The image indicates the discrimination between aorta samples of distal sections and proximal sections by difference of shades.

### DETAILED DESCRIPTION OF THE INVENTION

Genes whose expression is correlated with and determinant of an atherosclerotic phenotype or determinant of susceptibility to developing atherosclerosis are provided. The metagenes provided by the invention as useful in binary prediction tree statistical models to classify genes according to atherosclerotic phenotype or susceptibility to atherosclerosis. Also provided are methods of using the subject atherosclerotic determinant genes in diagnosis and treatment methods, as well as drug screening methods. In addition, reagents and kits thereof that find use in practicing the subject methods are provided. Also provided are methods of determining whether a gene is correlated with a disease phenotype, where correlation is determined using at least one parameter that is not expression level and is preferably determined using a binary prediction tree analysis.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to a collection of genes whose expression is correlated with atherosclerosis, i.e., that are atherosclerotic phenotype determinative genes, as well as methods for using the collection or subparts thereof in various applications. In further describing the invention, the collection of genes determinative of the atherosclerotic phenotype is described first in greater detail, followed by a review of the various different applications in which the collection finds use, including diagnostic, therapeutic and screening applications. Also reviewed are reagents and kits for use in practicing the subject methods. Finally, a review of various methods of identifying genes whose expression correlates with a given phenotype, such as atherosclerosis, is provided.

### Atherosclerotic Phenotype Determinative Genes

The subject invention provides a collection of atherosclerotic phenotype determinative genes. By atherosclerotic phenotype determinative genes is meant genes whose expression or lack thereof correlates with an atherosclerotic phenotype. Thus, atherosclerotic determinative genes include genes: (a) whose expression is correlated with an atherosclerotic phenotype, i.e., are expressed in cells and tissues thereof that have an atherosclerotic phenotype, and (b) whose lack of expression is correlated with an atherosclerotic phenotype, i.e., are not expressed in cells and tissues thereof that have an atherosclerotic phenotype. A cell is a cell with an atherosclerotic phenotype if it is obtained from vascular tissue that is determined to be atherosclerotic, e.g., by Sudan staining according to the method reported in the experimental section, below. Likewise, tissue is tissue with an atherosclerotic phenotype if it is vascular tissue or obtained from vascular tissue that is determined to be atherosclerotic, e.g., by Sudan staining according to the method reported in the experimental section, below.

By atherosclerotic susceptibility determinative genes is meant genes whose expression or lack thereof correlates with a susceptibility to developing an atherosclerotic phenotype.

The invention claims all collections and subsets thereof of atherosclerotic phenotype determinative genes as well as metagenes disclosed herewith. The subject collections of atherosclerotic phenotype determinative genes may be physical or virtual. Physical collections are those collections that include a population of different nucleic acid molecules, where the atherosclerotic phenotype determinative genes are represented in the population, i.e., there are nucleic acid molecules in the population that correspond in sequence to the genomic, or more typically, coding sequence of the atherosclerotic phenotype determinative genes in the collection.

In many embodiments, the nucleic acid molecules are either substantially identical or identical in sequence to the sense strand of the gene to which they correspond, or are complementary to the sense strand to which they correspond, typically to an extent that allows them to hybridize to their corresponding sense strand under stringent conditions. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

The nucleic acids that make up the subject physical collections may be single-stranded or double-stranded. In addition, the nucleic acids that make up the physical collections may be linear or circular, and the individual nucleic acid molecules may include, in addition to an atherosclerotic phenotype determinative gene coding sequence, other sequences, e.g., vector sequences. A variety of different nucleic acids may make up the physical collections, e.g., libraries, such as vector libraries, of the subject invention, where examples of different types of nucleic acids include, but are not limited to, DNA, e.g., cDNA, etc., RNA, e.g., mRNA, cRNA, etc. and the like. The nucleic acids of the physical collections may be present in solution or affixed, i.e., attached to, a solid support, such as a substrate as is found in array embodiments, where further description of such diverse embodiments is provided below.

Also provided are virtual collections of the subject atherosclerotic phenotype determinative genes. By virtual collection is meant one or more data files or other computer readable data organizational elements that include the sequence information of the genes of the collection, where the sequence information may be the genomic sequence information but is typically the coding sequence information. The virtual collection may be recorded on any convenient computer or processor readable storage medium. The computer or processor readable storage medium on which the collection data is stored may be any convenient medium, including CD, DAT, floppy disk, RAM, ROM, etc, which medium is capable of being read by a hardware component of the device.

Also provided are databases of expression profiles of atherosclerotic phenotype determinative genes. Such databases will typically comprise expression profiles of various cells/tissues having atherosclerotic phenotypes, such as various stages of atherosclerosis, negative expression profiles, prognostic profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks expression profiles possessing varying degrees of similarity to a reference expression profile. Such presentation provides a skilled artisan with a ranking of similarities and identifies the degree of similarity contained in the test expression profile.

Specific atherosclerotic phenotype determinative genes of the subject invention are those listed in Table I. Specific atherosclerotic susceptibility determinative genes of the subject invention are those listed in Table II. The invention also provides metagenes indicative of atherosclerotic burden or susceptibility. The subject collections and subsets thereof, as well as applications directed to the use of the aforementioned subject collections only serve as an example to illustrate the invention.

The subject collections of atherosclerotic-determinative genes include at least 2 of the genes listed in Table I. Table I contains the following genes, designated by Genbank Accession Number: J04765, AF052124, X15525, M94345, AB020687, U51240, Y09445, AF053233, U43185, AL050008, M12529, AB022718, Z24725, D13666, U50928, U46692, AF044253, J03909, L10333, M80634, M63138, J04430, AF044896, X78565, AB011143, X69819, J02947, U78095, D67029, AF013249, X62078, AB014574, L13939, D90144, L06797, D89077, Y08374, X02317, AB002365, AF084481, D34625, AB011103, AF041259, J05037, AF056087, U81800, AL050262, AB018271, AF014958, U58334, J03011, D12485, U22662, AF055008, D86964, U88629, U75308, J03600, AF004709, L15388, AB002361, X62744, AF072099, X90858, Z29067, U00952, J00194, M80254, M21121, AF030339, AJ007395, AF013570, Z22555, L22524, Y07512, Y00093, AB007889, Y08136, AB014520, L10678, Z98046, D79994, U29615, D87074, X81109, AL049946, U78556, U21931, M63603, D87433, AB029032, X12451, U89606, AB029018, M34423, AF095791, X03084, X74039, AB016811, X90976, U00802, X96752, Z49107, AL080235, AF051851, AF062075, AB000220, AF055581, AB015718, X78817, AJ000534, M63835, M16336, M32578, U32324, AF079167, M22324, D86961, X54162, U57911, M64571, AL023653, AL023653, AC005546, AC005546, M60028, Y13622, L76191, U83115, U60060, AJ011497, D64142, D26350, X15414, D87434, X79204, M58285, AB014513, U63127, S59184, X53587, Z84718, AF030409, J04621, AF055024, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, U15085, X97074, D89016, AF022797, M33552, U09578, M21186, M64925, AF029750, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663, M63193, D84110, AJ006973, AB002318, U51333, U09577 and U00672.

Preferred Table I genes consist of the following genes, designated by Genbank Accession Number: Y09445, AF053233, U43185, AL050008, AB022718, L10333, M80634, AF044896, X78565, AB011143, X69819, J02947, U78095, D67029, AF013249, AB014574, L13939, L06797, D89077, Y08374, X02317, AB002365, AF084481, D34625, AB011103, AF041259, J05037, AF056087, U81800, AL050262, AB018271, J03011, D12485, U88629, U75308, J03600, AF004709, AB002361, X90858, Z29067, U00952, M80254, AF030339, AJ007395, AF013570, Z22555, L22524, Y07512, Y00093, AB007889, Y08136, L10678, Z98046, D79994, D87074, X81109, AL049946, U78556, M63603, X12451, U89606, AB029018, AF095791, X74039, X90976, U00802, X96752, Z49107, AL080235, AF051851, AF062075, AB000220, AB015718, X78817, AJ000534, M63835, M16336, U32324, M22324, X54162, U57911, M64571, AC005546, AC005546, Y13622, L76191, U60060, AJ011497, D64142, D26350, X15414, D87434, X79204, AB014513, U63127, S59184, X53587, Z84718, AF030409, J04621, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, X97074, D89016, AF022797, M33552, U09578, M21186, M64925, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663, M63193, D84110, AJ006973, AB002318, U51333, U09577 and U00672.

Table II contains the following genes, designated by Genbank Accession Number: M26679, S82986, AF051323, J02947, M16937, K03000, M36711, D76435, M74297, M68891, U43328, X17360, X51757, U59831, D83004, L49169, L35545, U16799, M20560, X06256, Z22865, X75918, X16665, M97676, M55153. Preferred Table II genes consist of the following genes, designated by Genbank Accession Number: M68891, X51757, D83004, X06256, Z22865, X75918, M55153.

In certain embodiments, the number of genes in the collection that are from Table I or Table II is at least 5, at least 10, at least 25, at least 50, at least 75 or more, including all of the genes listed in Table I or are preferred Table I genes. The subject collections may include only those genes that are listed in Tables I and/or Table II, or they may include additional genes that are not listed in the tables. Where the subject collections include such additional genes, in certain embodiments the % number of additional genes that are present in the subject collections does not exceed about 50%, usually does not exceed about 25%. In many embodiments where additional “non-Table” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes.

In some embodiments, at least one of the genes in the collection is a gene whose function does not readily implicate it in the production of an atherosclerotic phenotype where such genes include those genes that are listed in Table I but which have not been assigned a biological process (see section 2 of the experimental section V for a listing of atherosclerotic determinative genes which have been assigned a biological functions; those not listed are the ones without a biological function assigned). In many embodiments, the subject collections include two or more genes from this group, where the number of genes that are included from this group may be 5, 10, 20 or more, up to and including all of the genes in this group. In some embodiments, the set comprises at least 1, 2, 3, 4, 5, 6, 7, 8, 9, 10, 12, 15, 20, 25, 30, 40 or 50 preferred genes from Table I.

In some embodiments, at least one of the genes in the collection is a gene whose function does not readily implicate it in susceptibility to atherosclerosis, where such genes include those genes that are listed in Table I but which have not been assigned a biological process (see section 3 of the experimental section V for a listing of atherosclerotic susceptibility genes which have been assigned a biological functions; those not listed are the ones without a biological function assigned). In many embodiments, the subject collections include 2 or more genes from this group, where the number of genes that are included from this group may be 5, 10, 20 or more, up to and including all of the genes in this group. In some embodiments, the set comprises at least 1, 2, 3, 4, 5, 6, 7, 8, 9, 10, 12, 15, 20, 25, 30, 40 or 50 preferred genes from Table II.

### Methods of Using the Subject Collections of Atherosclerotic Phenotype/Susceptibility Determinative Genes

The subject collections find use in a number of different applications. Applications of interest include, but are not limited to: (a) diagnostic applications, in which the collections of the genes are employed to either predict the presence of, or the probability for occurrence of, an atherosclerotic phenotype; (b) pharmacogenomic applications, in which the collections of genes are employed to determine an appropriate therapeutic treatment regimen, which is then implemented; and (c) therapeutic agent screening applications, where the collection of genes is employed to identify atherosclerotic phenotype modulatory agent or to identify atherosclerotic susceptibility modulatory agents. Each of these different representative applications is now described in greater detail below.

Diagnostic Applications

In diagnostic applications of the subject invention, cells or collections thereof, e.g., tissues, as well as animals (subjects, hosts, etc., e.g., mammals, such as pets, livestock, and humans, etc.) that include the cells/tissues are assayed to determine the presence of and/or probability for development of, an atherosclerotic phenotype. As such, diagnostic methods include methods of determining the presence of an atherosclerotic phenotype. In certain embodiments, not only the presence but also the severity or stage of an atherosclerotic phenotype is determined. In addition, diagnostic methods also include methods of determining the propensity to develop an atherosclerotic phenotype, such that a determination is made that an atherosclerotic phenotype is not present but is likely to occur.

In practicing the subject diagnostic methods, a nucleic acid sample obtained or derived from a cell, tissue or subject that includes the same that is to be diagnosed is first assayed to generate an expression profile, where the expression profile includes expression data for at least two of the genes of Table I or Table II, or preferred genes within those tables, where the expression profile may include expression data for 5, 10, 20, 50, 75 or more of, including all of, the genes listed in Table I or Table II, or preferred genes within those tables. In many embodiments, the expression profile also includes expression data for at least 1 of the genes listed in Table I or Table II, or preferred genes within those tables, wherein the expression profile may include expression data for 2, 5, 10, 20 or more, including all of the genes listed in Table I or Table II, or preferred genes within those tables. The number of different genes whose expression data, i.e., presence or absence of expression, as well as expression level, that are included in the expression profile that is generated may vary, but is typically at least 2, and in many embodiments ranges from 2 to about 100 or more, sometimes from 3 to about 75 or more, including from about 4 to about 70 or more.

As indicated above, the sample that is assayed to generate the expression profile employed in the diagnostic methods is one that is a nucleic acid sample. The nucleic acid sample includes a plurality or population of distinct nucleic acids that includes the expression information of the atherosclerotic phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined atherosclerotic phenotype exists, including, but not limited, to, monocytes, endothelium, and/or smooth muscle.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the atherosclerotic phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Once the expression profile is obtained from the sample being assayed, the expression profile is compared with a reference or control profile to make a diagnosis regarding the atherosclerotic phenotype of the cell or tissue from which the sample was obtained/derived. The reference or control profile may be a profile that is obtained from a cell/tissue known to have an atherosclerotic phenotype, as well as a particular stage of atherosclerosis, and therefore may be a positive reference or control profile. In addition, the reference or control profile may be a profile from cell/tissue for which it is known that the cell/tissue ultimately developed an atherosclerotic phenotype, and therefore may be a positive prognostic control or reference profile. In addition, the reference/control profile may be from a normal cell/tissue and therefore be a negative reference/control profile.

In certain embodiments, the obtained expression profile is compared to a single reference/control profile to obtain information regarding the atherosclerotic phenotype of the cell/tissue being assayed. In yet other embodiments, the obtained expression profile is compared to two or more different reference/control profiles to obtain more in depth information regarding the atherosclerotic phenotype of the assayed cell/tissue. For example, the obtained expression profile may be compared to a positive and negative reference profile to obtain confirmed information regarding whether the cell/tissue has an atherosclerotic or normal phenotype. Furthermore, the obtained expression profile may be compared to a series of positive control/reference profiles each representing a different stage/level of atherosclerosis, so as to obtain more in depth information regarding the particular atherosclerotic phenotype of the assayed cell/tissue. The obtained expression profile may be compared to a prognostic control/reference profile, so as to obtain information about the propensity of the cell/tissue to develop an atherosclerotic phenotype.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the control/reference profiles, which similarity/dissimilarity information is employed to determine the atherosclerotic phenotype of the cell/tissue being assayed. For example, similarity with a positive control indicates that the assayed cell/tissue has an atherosclerotic phenotype. Likewise, similarity with a negative control indicates that the assayed cell/tissue does not have an atherosclerotic phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of an atherosclerotic phenotype of an assayed cell/tissue. In addition, where appropriate reference profiles are employed, the above comparison step can yield information about the particular stage of an atherosclerotic phenotype of an assayed cell/tissue. Furthermore, the above comparison step can be used to obtain information regarding the propensity of the cell or tissue to develop an atherosclerotic phenotype.

In many embodiments, the above obtained information about the cell/tissue being assayed is employed to diagnose a host, subject or patient with respect to the presence of, state of or propensity to develop, atherosclerosis. For example, where the cell/tissue that is assayed is determined to have an atherosclerotic phenotype, the information may be employed to diagnose a subject from which the cell/tissue was obtained as having atherosclerosis.

Pharmaco/Surgicogenomic Applications

Another application in which the subject collections of atherosclerotic phenotype determinative genes finds use in is pharmacogenomic and/or surgicogenomic applications. In these applications, a subject/host/patient is first diagnosed for an atherosclerotic phenotype, e.g., presence or absence of atherosclerosis, propensity to develop atherosclerosis, etc., using a protocol such as the diagnostic protocol described in the preceding section.

The subject is then treated using a pharmacological and/or surgical treatment protocol, where the suitability of the protocol for a particular subject/patient is determined using the results of the diagnosis step. A variety of different pharmacological and surgical treatment protocols are known to those of skill in the art. Such protocols include, but are not limited to: surgical treatment protocols, including bypass grafting, endarterectomy, and percutaneous translumenal angioplasty (PCTA). Pharmacological protocols of interest include treatment with a variety of different types of agents, including but not limited to: thrombolytic agents, growth factors, cytokines, nucleic acids (e.g. gene therapy agents); etc.

Assessment of Therapy (Therametrics)

Another application in which the subject collections of atherosclerotic phenotype determinative genes and/or atherosclerotic phenotype susceptibility genes find use is in monitoring or assessing a given treatment protocol. In such methods, a cell/tissue sample of a patient undergoing treatment for an atherosclerosis disease condition is monitored using the procedures described above in the diagnostic section, where the obtained expression profile is compared to one or more reference profiles to determine whether a given treatment protocol is having a desired impact on the disease being treated. For example, periodic expression profiles are obtained from a patient during treatment and compared to a series of reference/controls that includes expression profiles of various atherosclerotic stages and normal expression profiles. An observed change in the monitored expression profile towards a normal profile indicates that a given treatment protocol is working in a desired manner.

Therapeutic Agent Screening Applications

The present invention also encompasses methods for identification of agents having the ability to modulate an atherosclerotic phenotype, e.g., enhance or diminish an atherosclerotic phenotype, which finds use in identifying therapeutic agents for atherosclerosis.

Identification of compounds that modulate an atherosclerotic phenotype can be accomplished using any of a variety of drug screening techniques. The screening assays of the invention are generally based upon the ability of the agent to modulate an expression profile of atherosclerotic phenotype determinative genes.

The term “agent” as used herein describes any molecule, e.g., protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Exemplary candidate agents of particular interest include, but are not limited to, antisense polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

Screening assays can be based upon any of a variety of techniques readily available and known to one of ordinary skill in the art. In general, the screening assays involve contacting a cell or tissue known to have an atherosclerotic phenotype with a candidate agent, and assessing the effect upon a gene expression profile made up of atherosclerotic phenotype determinative genes. The effect can be detected using any convenient protocol, where in many embodiments the diagnostic protocols described above are employed. Generally such assays are conducted in vitro, but many assays can be adapted for in vivo analyses, e.g., in an animal model of the diabetes.

Screening for Drug Targets

In another embodiment, the invention contemplates identification of genes and gene products from the subject collections of atherosclerotic determinative genes or of atherosclerosis susceptibility genes as therapeutic targets. In some respects, this is the converse of the assays described above for identification of agents having activity in modulating (e.g., decreasing or increasing) an atherosclerotic phenotype, and is directed towards identifying genes that are atherosclerotic phenotype determinative genes, e.g., the genes appearing in Table I, as therapeutic targets or directed towards identifying genes that are atherosclerotic susceptibility determinative genes, e.g., the genes appearing in Table II, as therapeutic targets

In this embodiment, therapeutic targets are identified by examining the effect(s) of an agent that can be demonstrated or has been demonstrated to modulate an atherosclerotic phenotype (e.g., inhibit or suppress an atherosclerotic phenotype). For example, the agent can be an antisense oligonucleotide that is specific for a selected gene transcript. For example, the antisense oligonucleotide may have a sequence corresponding to a sequence of a gene appearing in Table I or Table II or preferred genes within these tables.

Assays for identification of therapeutic targets can be conducted in a variety of ways using methods that are well known to one of ordinary skill in the art. For example, a test cell that expresses or overexpresses a candidate gene, e.g., a gene found in Table I, is contacted with the known atherosclerotic agent, the effect upon a atherosclerotic phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product.

Inhibition or suppression of the atherosclerotic phenotype indicates that the candidate gene product is a suitable target for atherosclerotic therapy. Assays described herein and/or known in the art can be readily adapted in for assays for identification of therapeutic targets. Generally such assays are conducted in vitro, but many assays can be adapted for in vivo analyses, e.g., in an appropriate, art-accepted animal model of atherosclerosis.

### Reagents and Kits

Also provided are reagents and kits thereof for practicing one or more of the above described methods. The subject reagents and kits thereof may vary greatly. Reagents of interest include reagents specifically designed for use in production of the above described expression profiles of atherosclerotic phenotype determinative genes.

One type of such reagent is an array probe nucleic acids in which the atherosclerotic phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In many embodiments, the arrays include probes for at least 2 of the genes listed in Table I or Table II, or preferred genes listed therein. In certain embodiments, the number of genes that are from Table I that is represented on the array is at least 5, at least 10, at least 25, at least 50, at least 75 or more, including all of the genes listed in Table I and/or Table II. The subject arrays may include only those genes that are listed in Table I and/or Table II, or they may include additional genes that are not listed in Table I and Table II. Where the subject arrays include probes for such additional genes, in certain embodiments the number % of additional genes that are represented does not exceed about 50%, usually does not exceed about 25%. In many embodiments where additional “non-Table I or Table II” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes. In many embodiments, at least one of the genes represented on the array is a gene whose function does not readily implicate it in the production of an atherosclerotic phenotype, where such genes include those genes listed in Table I and/or Table II. In many embodiments, the subject arrays include 2 or more genes from Table I and/or Table II, where the number of genes that are included from Table I may be 5, 10, 20 or more, up to an including all of the genes listed in Table I and/or Table II.

Another type of reagent that is specifically tailored for generating expression profiles of atherosclerotic phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 2 of the genes listed in Table I and/or Table II, above. In certain embodiments, the number of genes that are from Table I and/or Table II that have primers in the collection is at least 5, at least 10, at least 25, at least 50, at least 75 or more, including all of the genes listed in Table I and/or Table II. The subject gene specific primer collections may include only those genes that are listed in Table I and/or Table II, or they may include primers for additional genes that are not listed in Table I and/or Table II. Where the subject gene specific primer collections include primers for such additional genes, in certain embodiments the number % of additional genes that are represented does not exceed about 50%, usually does not exceed about 25%. In many embodiments where additional “non-Table I and/or Table II” genes are included, a great majority of genes in the collection are atherosclerotic phenotype determinative genes, where by great majority is meant at least about 75%, usually at least about 80% and sometimes at least about 85, 90, 95% or higher, including embodiments where 100% of the genes in the collection are atherosclerotic phenotype determinative genes. In many embodiments, at least one of the genes represented on collection of gene specific primers is a gene whose function does not readily implicate it in the production of an atherosclerotic phenotype, where such genes include those genes listed in Table I and/or Table II. In many embodiments, the subject gene specific primer collections include 2 or more genes from Table I and/or Table II, where the number of genes that are included from Table I and/or Table II may be 5, 10, 20 or more, up to an including all of the genes listed in Table I and/or Table II.

The kits of the subject invention may include the above described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Compounds and Methods for Treatment of Cardiovascular Disease

Also provided are methods and compositions whereby cardiovascular disease symptoms may be ameliorated. The subject invention provides methods of ameliorating, e.g., treating, an atherosclerotic disease conditions, by modulating the expression of one or more target genes or the activity of one or more products thereof, where the target genes are one or more of the atherosclerotic phenotype determinative genes of Table I, the preferred genes of Table I, or the genes in Table I to which no function has been assigned. The subject invention provides methods of decreasing the susceptibility to atherosclerotic disease by modulating the expression of one or more target genes or the activity of one or more products thereof, where the target genes are one or more of the atherosclerotic susceptibility determinative genes of Table II, the preferred genes of Table II, or the genes in Table II to which no function has been assigned.

Certain cardiovascular diseases are brought about, at least in part, by an excessive level of gene product, or by the presence of a gene product exhibiting an abnormal or excessive activity. As such, the reduction in the level and/or activity of such gene products would bring about the amelioration of cardiovascular disease symptoms. Techniques for the reduction of target gene expression levels or target gene product activity levels are discussed below.

Alternatively, certain other cardiovascular diseases are brought about, at least in part, by the absence or reduction of the level of gene expression, or a reduction in the level of a gene product's activity. As such, an increase in the level of gene expression and/or the activity of such gene products would bring about the amelioration of cardiovascular disease symptoms. Techniques for increasing target gene expression levels or target gene product activity levels are discussed below.

### Compounds that Inhibit Expression, Synthesis or Activity of Mutant Target Gene Activity

As discussed above, target genes involved in cardiovascular disease disorders can cause such disorders via an increased level of target gene activity. A variety of techniques may be utilized to inhibit the expression, synthesis, or activity of such target genes and/or proteins. For example, compounds such as those identified through assays described which exhibit inhibitory activity, may be used in accordance with the invention to ameliorate cardiovascular disease symptoms. As discussed, above, such molecules may include, but are not limited to small organic molecules, peptides, antibodies, and the like. Inhibitory antibody techniques are described, below.

For example, compounds can be administered that compete with an endogenous ligand for the target gene product, where the target gene product binds to an endogenous ligand. The resulting reduction in the amount of ligand-bound gene target will modulate endothelial cell physiology. Compounds that can be particularly useful for this purpose include, for example, soluble proteins or peptides, such as peptides comprising one or more of the extracellular domains, or portions and/or analogs thereof, of the target gene product, including, for example, soluble fusion proteins such as Ig-tailed fusion proteins. (For a discussion of the production of Ig-tailed fusion proteins, see, for example, U.S. Pat. No. 5,116,964.). Alternatively, compounds, such as ligand analogs or antibodies, that bind to the target gene product receptor site, but do not activate the protein, (e.g., receptor-ligand antagonists) can be effective in inhibiting target gene product activity. Furthermore, antisense and ribozyme molecules which inhibit expression of the target gene may also be used in accordance with the invention to inhibit the aberrant target gene activity. Such techniques are described, below. Still further, also as described, below, triple helix molecules may be utilized in inhibiting the aberrant target gene activity.

### Inhibitory Antisense, Ribozyme and Triple Helix Approaches

Among the compounds which may exhibit the ability to ameliorate cardiovascular disease symptoms are antisense, RNA interference molecules (RNAi), ribozyme, and triple helix molecules. Such molecules may be designed to reduce or inhibit mutant target gene activity. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

“RNA interference” or “RNAi” is a term initially applied to a phenomenon observed in plants and worms where double-stranded RNA (dsRNA) blocks gene expression in a specific and post-transcriptional manner. Current research indicates that RNAi appears to involve mRNA degradation. Despite some uncertainty regarding the mechanism of action, RNAi provides a useful method of inhibiting gene expression in vitro or in vivo. RNAi constructs comprise double stranded RNA that can specifically block expression of a target gene. “RNAi construct” is a generic term used herein to include small interfering RNAs (siRNAs, around 19-30 nucleotides in length), hairpin RNAs, and other RNA species which can be cleaved in vivo to form siRNAs. RNAi constructs also include expression vectors (also referred to as RNAi expression vectors) capable of giving rise to transcripts which form dsRNAs or hairpin RNAs in cells, and/or transcripts which can produce siRNAs in vivo. RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 base pairs, or 1 in 10 base pairs, or 1 in 20 base pairs, or 1 in 50 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Exemplary methods of making and delivering either long or short RNAi constructs can be found, for example, in WO01/68836 and WO01/75164. RNAi constructs can be synthesized using methods well known in the art to synthesize or recombinantly produce RNA molecules.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

It is possible that the antisense, RNAi, ribozyme, and/or triple helix molecules described herein may reduce or inhibit the transcription (triple helix) and/or translation (antisense, ribozyme) of mRNA produced by both normal and mutant target gene alleles. To ensure that substantially normal levels of target gene activity are maintained, nucleic acid molecules that encode and express target gene polypeptides exhibiting normal activity may be introduced into cells via gene therapy methods such as those described, below, that do not contain sequences susceptible to whatever antisense, RNAI, ribozyme, or triple helix treatments are being utilized. Alternatively, it may be preferable to co-administer normal target gene protein into the cell or tissue to maintain the requisite level of cellular or tissue target gene activity.

Anti-sense RNA and DNA, RNAi constructs, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Antibodies for Target Gene Products

Antibodies that are both specific for target gene protein and interfere with its activity may be used to inhibit target gene function. Such antibodies may be generated using standard techniques known in the art against the proteins themselves or against peptides corresponding to portions of the proteins. Such antibodies include but are not limited to polyclonal, monoclonal, Fab fragments, single chain antibodies, chimeric antibodies, etc.

In instances where the target gene protein is intracellular and whole antibodies are used, internalizing antibodies may be preferred. However, lipofectin liposomes may be used to deliver the antibody or a fragment of the Fab region which binds to the target gene epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Creighton, 1983, supra; and Sambrook et al., 1989, supra). Alternatively, single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (Marasco, W. et al., 1993, Proc. Natl. Acad. Sci. USA 90:7889-7893).

In some instances, the target gene protein is extracellular, or is a transmembrane protein. Antibodies that are specific for one or more extracellular domains of the gene product, for example, and that interfere with its activity, are particularly useful in treating cardiovascular disease. Such antibodies are especially efficient because they can access the target domains directly from the bloodstream. Any of the administration techniques described, below which are appropriate for peptide administration may be utilized to effectively administer inhibitory target gene antibodies to their site of action.

### Methods for Restoring Target Gene Activity

Target genes that cause cardiovascular disease may be underexpressed within cardiovascular disease situations. Alternatively, the activity of target gene products may be diminished, leading to the development of cardiovascular disease symptoms. Described in this Section are methods whereby the level of target gene activity may be increased to levels wherein cardiovascular disease symptoms are ameliorated. The level of gene activity may be increased, for example, by either increasing the level of target gene product present or by increasing the level of active target gene product which is present.

For example, a target gene protein, at a level sufficient to ameliorate cardiovascular disease symptoms may be administered to a patient exhibiting such symptoms. Any of the techniques discussed, below, may be utilized for such administration. One of skill in the art will readily know how to determine the concentration of effective, non-toxic doses of the normal target gene protein, utilizing techniques known to those of ordinary skill in the art.

Additionally, RNA sequences encoding target gene protein may be directly administered to a patient exhibiting cardiovascular disease symptoms, at a concentration sufficient to produce a level of target gene protein such that cardiovascular disease symptoms are ameliorated. Any of the techniques discussed, below, which achieve intracellular administration of compounds, such as, for example, liposome administration, may be utilized for the administration of such RNA molecules. The RNA molecules may be produced, for example, by recombinant techniques as is known in the art.

Further, patients may be treated by gene replacement therapy. One or more copies of a normal target gene, or a portion of the gene that directs the production of a normal target gene protein with target gene function, may be inserted into cells using vectors which include, but are not limited to adenovirus, adeno-associated virus, and retrovirus vectors, in addition to other particles that introduce DNA into cells, such as liposomes. Additionally, techniques such as those described above may be utilized for the introduction of normal target gene sequences into human cells.

Cells, preferably, autologous cells, containing normal target gene expressing gene sequences may then be introduced or reintroduced into the patient at positions which allow for the amelioration of cardiovascular disease symptoms. Such cell replacement techniques may be preferred, for example, when the target gene product is a secreted, extracellular gene product.

### Pharmaceutical Preparations and Methods of Administration

The identified compounds that inhibit target gene expression, synthesis and/or activity can be administered to a patient at therapeutically effective doses to treat or ameliorate cardiovascular disease. A therapeutically effective dose refers to that amount of the compound sufficient to result in amelioration of symptoms of cardiovascular disease.

1. Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations and Use

Pharmaceutical compositions for use in accordance with the present invention may be formulated in conventional manner using one or more physiologically acceptable carriers or excipients. Suitable vehicles and their formulation inclusive of various proteins are described, for example, in the book Remington's Pharmaceutical Sciences (Mack Publishing Company, Easton, Pa., USA 1985) or Handbook of Pharmaceutical Excipients, 4^{th} ed. (Ed. Rowe et al., Pharmaceutical Press, Grayslake, Ill., USA 2003), the contents of which are incorporated herein by reference.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Methods of Identifying Atherosclerotic Phenotype/Susceptibility Determinative Genes

Also provided are methods of identifying atherosclerotic phenotype determinative genes, i.e., genes whose expression is associated with a disease phenotype, or of identifying atherosclerotic susceptibility determinative genes, i.e., genes whose expression is associated with an atherosclerotic susceptibility phenotype.

In these methods, an expression profile for a nucleic acid sample obtained from a source having the atherosclerotic phenotype, or a sample to be tested for susceptibility, is prepared using the gene expression profile generation techniques described above, with the only difference being that the genes that are assayed are candidate genes and not genes necessarily known to be atherosclerotic phenotype/susceptibility determinative genes. Next, the obtained expression profile is compared to a control profile, e.g., obtained from a source that does not have an atherosclerotic phenotype.

Following this comparison step, genes whose expression correlates with said the atherosclerotic phenotype/susceptibility are identified. A feature of the subject invention is that the correlation is based on at least one parameter that is other than expression level. As such, a parameter other than whether a gene is up or down regulated is employed to find a correlation of the gene with the atherosclerotic phenotype.

One expression analysis approach may include a Bayesian analysis of binary prediction tree models for retrospectively sampled outcomes as illustrated in the following three exemplary analyses.

Bayesian analysis is an approach to statistical analysis that is based on the Bayes law, which states that the posterior probability of a parameter p is proportional to the prior probability of parameter p multiplied by the likelihood of p derived from the data collected. This increasingly popular methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon. Bayesian analysis have been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” modes using a splitting rule. For instance, for clarification, training data contains sample vectors that have one or more measurement variables and one variable that determines that class of the sample. Various splitting rules have been used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. A statistical predictive tree model to which Bayesian analysis is applied may consistently deliver accurate results with high predictive capabilities.

Development of the Tree Clarification Model: Model Context and Methodology

Data {Zi, xᵢ} (i=1, . . . , n) are available on a binary response variable Z and a p-dimensional covariate vector x: The 0/1 response totals are fixed by design. Each predictor variable xⱼ could be binary, discrete or continuous.

1. Bayes' Factor Measures of Association

At the heart of a classification tree is the assessment of association between each predictor and the response in subsamples, and we first consider this at a general level in the full sample. For any chosen single predictor x; a specified threshold_on the levels of x organizes the data into the 2x2 table.

| | Z = 0 | Z = 1 | |
|---|---|---|---|
| x ≦ T | n₀₀ | n₀₁ | N₀ |
| x > T | n₁₀ | n₁₁ | N₁ |
| | M₀ | M₁ | |

With column totals fixed by design, the categorized data is properly viewed as two Bernoulli sequences within the two columns, hence sampling
p(n_{0z},n_{1z}|M_{z},θ_{z,σ})=θ_{z,τ}ⁿ^{_{0z}}(1−θ_{z,τ})ⁿ^{_{1z}}
for each column z=0, 1. Here, of coursed θ_{0,τ}=Pr(x≦τ|Z=0) and θ_{1,τ}=Pr(x≦τ|Z=1). A test of association of the thresholded predictor with the response will now be based on assessing the difference between these Bernoulli probabilities.

The natural Bayesian approach is via the Bayes' factor β_{τ} comparing the null hypothesis θ_{0,τ}=θ_{1,τ} to the full alternative θ_{0,τ}≠θ_{1,τ}. We adopt the standard conjugate beta prior model and require that the null hypothesis be nested within the alternative. Thus, assuming θ_{0,τ}≠θ_{1,τ}, we take θ_{0,τ and θ}_{1,τ} to be independent with common prior Be(a_{τ},b_{τ}) with mean m_{τ}=a_{τ}/(a_{τ}+b_{τ}). On the null hypothesis θ_{0,τ}=θ_{1,τ}, the common value has the same beta prior. The resulting Bayes' factor in favour of the alternative over the null hypothesis is then simply

$${B}_{τ} = \frac{β ( {n}_{00} + {a}_{τ} , {n}_{10} + {b}_{τ} ) β ( {n}_{01} + {a}_{τ} , {n}_{11} + {b}_{τ} )}{β ( {N}_{0} + {a}_{τ} , {N}_{1} + {b}_{τ} ) β ( {a}_{τ} {b}_{τ} )} .$$

As a Bayes' factor, this is calibrated to a likelihood ratio scale. In contrast to more traditional significance tests and also likelihood ratio approaches, the Bayes' factor will tend to provide more conservative assessments of significance, consistent with the general conservative properties of proper Bayesian tests of null hypotheses (See Sellke, T., Bayarri, M. J. and Berger, J. O., Calibration of p_values for testing precise null hypotheses, The American Statistician, 55, 62-71, (2001) and references therein).

In the context of comparing predictors, the Bayes' factor Bτ may be evaluated for all predictors and, for each predictor, for any specified range of thresholds. As the threshold varies for a given predictor taking a range of (discrete or continuous) values, the Bayes' factor maps out a function of τ and high values identify ranges of interest for thresholding that predictor. For a binary predictor, of course, the only relevant threshold to consider is τ=0.

2. Model Consistency with Respect to Varying Thresholds

A key question arises as to the consistency of this analysis as we vary the thresholds. By construction, each probability θ_{Zτ} is a non-decreasing function of τ, a constraint that must be formally represented in the model. The key point is that the beta prior specification must formally reflect this. To see how this is achieved, note first that θ_{Zτ} is in fact the cumulative distribution function of the predictor values χ; conditional on Z=z; (z=0; 1); evaluated at the point χ=τ. Hence the sequence of beta priors, Be(a_{τ}, b_{τ}) as τ varies, represents a set of marginal prior distributions for the corresponding set of values of the cdfs. It is immediate that the natural embedding is in a non-parametric Dirichlet process model for the complete cdf. Thus the threshold-specific beta priors are consistent, and the resulting sets of Bayes' factors comparable as τ varies, under a Dirichlet process prior with the betas as margins. The required constraint is that the prior mean values m_{τ} are themselves values of a cumulative distribution function on the range of χ, one that defines the prior mean of each θ_{τ} as a function. Thus, we simply rewrite the beta parameters (α_{τ}, b_{τ}) as α_{τ}=αm_{τ} and b_{τ}=α(1−m_{τ}) for a specified prior mean cdf m_{τ}, and where α is the prior precision (or “total mass”) of the underlying Dirichlet process model. Note that this specializes to a Dirichlet distribution when χ is discrete on a finite set of values, including special cases of ordered categories (such as arise if χ is truncated to a predefined set of bins), and also the extreme case of binary χ when the Dirichlet is a simple beta distribution.

3. Generating a Tree

The above development leads to a formal Bayes' factor measure of association that may be used in the generation of trees in a forward-selection process as implemented in traditional classification tree approaches. Consider a single tree and the data in a node that is a candidate for a binary split. Given the data in this node, construct a binary split based on a chosen (predictor, threshold) pair (ω, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 3 prior, Bayes' factors of 2.2, 2.9, 3.7 and 5.3 correspond, approximately, to probabilities of 0.9, 0.95, 0.99 and 0.995, respectively. This guides the choice of threshold, which may be specified as a single value for each level of the tree. We have utilized Bayes' factor thresholds of around 3 in a range of analyses, as exemplified below. Higher thresholds limit the growth of trees by ensuring a more stringent test for splits.

The Bayes' factor measure will always generate less extreme values than corresponding generalized likelihood ratio tests (for example), and this can be especially marked when the sample sizes M₀ and M₁ are low. Thus the propensity to split nodes is always generally lower than with traditional testing methods, especially with lower samples sizes, and hence the approach tends to be more conservative in extending existing trees. Post-generation pruning is therefore generally much less of an issue, and can in fact generally be ignored. Index the root node of any tree by zero, and consider the full data set of n observations, representing M_{z} outcomes with Z=z in 0, 1. Label successive nodes sequentially: splitting the root node, the left branch terminates at node 1, the right branch at node 2; splitting node 1, the consequent left branch terminates at node 3, the right branch at node 4; splitting node 2, the consequent left branch terminates at node 5, and the right branch at node 6, and so forth. Any node in the tree is labelled numerically according to its “parent” node; that is, a node j splits into two children, namely the (left, right) children (2j+1; 2j+2): At level m of the tree (m=0; 1; : : : ;) the candidates nodes are, from left to right, as 2^{m}_{—}1; 2^{m}; : : : ; 2^{m+1}−2.

Having generated a “current” tree, we run through each of the existing terminal nodes one at a time, and assess whether or not to create a further split at that node, stopping based on the above Bayes' factor criterion. Unless samples are very large (thousands) typical trees will rarely extend to more than three or four levels.

4. Inference and Prediction with a Single Tree

Suppose we have generated a tree with m levels; the tree has some number of terminal nodes up to the maximum possible of L=₂ m+1-2. Inference and prediction involves computations for branch probabilities and the predictive probabilities for new cases that these underlie. We detail this for a specific path down the tree, i.e., a sequence of nodes from the root node to a specified terminal node.

First, consider a node j that is split based on a (predictor, threshold) pair labeled (χⱼ, τⱼ), (note that we use the node index to label the chosen predictor, for clarity). Extend the notation of Section 2.1 to include the subscript j indexing this node. Then the data at this node involves M₀ⱼ cases with Z=0 and M₁ⱼ cases with Z=1. Based on the chosen (predictor, threshold) pair (χⱼ, τⱼ) these samples split into cases n₀₀ⱼ, n₀₁ⱼ, n₁₀ⱼ, n₁₁ⱼ as in the table of Section 2.1, but now indexed by the node label j. The implied conditional probabilities θ_{z,τ,j}=Pr(χⱼ≦τⱼ|Z=z), for z=0, 1 are the branch probabilities defined by such a split (note that these are also conditional on the tree and data subsample in this node, though the notation does not explicitly reflect this for clarity). These are uncertain parameters and, following the development of Section 2.1, have specified beta priors, now also indexed by parent node j, i.e., Be(a_{τ,j}, b_{τ,j}). Assuming the node is split, the two sample Bernoulli setup implies conditional posterior distributions for these branch probability parameters: they are independent with posterior beta distributions
θ_{0,τ,j}˜Be(a_{τ,j}+n₀₀ⱼ, b_{τ,j}+n₁₀ⱼ) and θ_{1,τj}˜Be(a_{τ,j}+n₀₁ⱼ, b_{τ,j}+n₁₁ⱼ).

These distributions allow inference on branch probabilities, and feed into the predictive inference computations as follows.

Consider predicting the response Z* of a new case based on the observed set of predictor values x*. The specified tree defines a unique path from the root to the terminal node for this new case. To predict requires that we compute the posterior predictive probability for Z*=1/0. We do this by following x* down the tree to the implied terminal node, and sequentially building up the relevant likelihood ratio defined by successive (predictor, threshold) pairs.

For example and specificity, suppose that the predictor profile of this new case is such that the implied path traverses nodes 0, 1, 4, 9, terminating at node 9. This path is based on a (predictor, threshold) pair (χ₀, τ₀) that defines the split of the root node, (χ₁, τ₁) that defines the split of node 1, and (χ₄, τ₄) that defines the split of node 4. The new case follows this path as a result of its predictor values, in sequence: (x₀*≦τ₀): (x₁*>τ₁) and (x₄*≦τ₄). The implied likelihood ratio for Z*=1 relative to Z*=0 is then the product of the ratio of branch probabilities to this terminal node, namely

$${λ}^{*} = \frac{{θ}_{1 , {τ}_{0} , 0}}{{θ}_{0 , {τ}_{0} , 0}} \times \frac{( 1 - {θ}_{1 , {τ}_{1} , 1} )}{( 1 - {θ}_{0 , {τ}_{1} , 1} )} \times \frac{{θ}_{1 , {τ}_{0} , 0}}{{θ}_{0 , {τ}_{0} , 0}}$$

Hence, for any specified prior probability Pr(Z*=1), this single tree model implies that, as a function of the branch probabilities, the updated probability π* is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )} .$$

Hence, for any specified prior probability π Pr(Z*=1), this single tree model implies that, as a function the branch probabilities, the updated probability π* is, on the odds scale, given by

$$\frac{{π}^{*}}{( 1 - {π}^{*} )} = \frac{λ * Pr ( {Z}^{*} = 1 )}{Pr ( {Z}^{*} = 0 )}$$

The case-control design provides no information about Pr(Z*=1) so it is up to the user to specify this or examine a range of values; one useful summary is obtained by simply taking a 50:50 prior odds as benchmark, whereupon the posterior probability is
π*=λ*/(1+λ*).
Prediction follows by estimating π* based on the sequence of conditionally independent posterior distributions for the branch probabilities that define it. For example, simply “plugging-in” the conditional posterior means of each θ. will lead to a plug-in estimate of λ* and hence π*. The full posterior for π* is defined implicitly as it is a function of the θ. Since the branch probabilities follow beta posteriors, it is trivial to draw Monte Carlo samples of the θ. and then simply compute the corresponding values of λ* and hence π* to generate a posterior sample for summarization. This way, we can evaluate simulation-based posterior means and uncertainty intervals for π* that represent predictions of the binary outcome for the new case.
5. Generating and Weighting Multiple Trees

In considering potential (predictor, threshold) candidates at any node, there may be a number with high Bayes' factors, so that multiple possible trees with difference splits at this node are suggested. With continuous predictor variables, small variations in an “interesting” threshold will generally lead to small changes in the Bayes' factor—moving the threshold so that a single observation moves from one side of the threshold to the other, for example. This relates naturally to the need to consider thresholds as parameters to be inferred; for a given predictor χ, multiple candidate splits with various different threshold values τ reflects the inherent uncertainty about τ, and indicates the need to generate multiple trees to adequately represent that uncertainty. Hence, in such a situation, the tree generation can spawn multiple copies of the “current” tree, and then each will split the current node based on a different threshold for this predictor. Similarly, multiple trees may be spawned this way with the modification that they may involve different predictors.

In problems with many predictors, this naturally leads to the generation of many trees, often with small changes from one to the next, and the consequent need for careful development of tree-managing software to represent the multiple trees. In addition, there is then a need to develop inference and prediction in the context of multiple trees generated this way. The use of “forests of trees” has recently been urged by Breiman, L., Statistical Modeling: The two cultures (with discussion), Statistical Science, 16 199-225 (2001), and our perspective endorses this. The rationale here is quite simple: node splits are based on specific choices of what we regard as parameters of the overall predictive tree model, the (predictor, threshold) pairs. Inference based on any single tree chooses specific values for these parameters, whereas statistical learning about relevant trees requires that we explore aspects of the posterior distribution for the parameters (together with the resulting branch probabilities).

Within the current framework, the forward generation process allows easily for the computation of the resulting relative likelihood values for trees, and hence to relevant weighting of trees in prediction. For a given tree, identify the subset of nodes that are split to create branches. The overall marginal likelihood function for the tree is then the product of component marginal likelihoods, one component from each of these split nodes. Continue with the notation of Section 2.1 but now, again, indexed by any chosen node j: Conditional on splitting the node at the defined (predictor, threshold) pair (χⱼ, τⱼ), the marginal likelihood component is

$${m}_{j} = {\int }_{0}^{1} {\int }_{0}^{1} {\prod }_{z = 0 , 1}^{} p ( {n}_{0 zj} , {n}_{1 zj} \text{\vert} {M}_{zj} , {θ}_{z , {τ}_{j} , j} ) p ( {θ}_{z , {τ}_{j} , j} ) ⅆ {θ}_{z , {τ}_{j} , j}$$

where p(θ_{z,τ}_{ⱼ}ⱼ) is the Be(a_{τj}, b_{τj}) prior for each z=0, 1. This clearly reduces to

$${m}_{j} = {\prod }_{z = 0 , 1}^{} \frac{β ( {n}_{0 zj} + {a}_{τ , j} , {n}_{1 zj} + {b}_{τ j} )}{β ( {a}_{τ , j} , {b}_{τ , j} )} .$$

The overall marginal likelihood value is the product of these terms over all nodes j that define branches in the tree. This provides the relative likelihood values for all trees within the set of trees generated. As a first reference analysis, we may simply normalize these values to provide relative posterior probabilities over trees based on an assumed uniform prior. This provides a reference weighting that can be used to both assess trees and as posterior probabilities with which to weight and average predictions for future cases.

### EXAMPLE 1

### Analysis of Biscuit Dough Data

A first example concerns the application of biscuit dough data (publicly available at Osborne, B. G., Fearn, T., Miller, A. R. and Douglas, S., Applications of near infrared reflectance spectroscopy to compositional analysis of biscuits and biscuit doughs, J. Sci. Food Agric., 35, 99-105 (1984); Brown, P. J., Fearn, T. and Vannucci, M., The choice of variables in multivariate regression: A non-conjugate Bayesian decision theory approach, Biometrika, 86, 635-648 (1999)) in which interest lies in relating aspects of near infrared (“NIR”) spectra of dough to the fat content of the resulting biscuits. The data set provides 78 samples, of which 39 are taken as training data and the remaining 39 as validation cases to be predicted, precisely as in Brown et al (1999). The binary outcome is 0/1 according to whether the measured fat content exceeds a threshold, where the threshold is the mean of the sample of fat values. As predictors, each xi comprises 300 values of the spectrum of dough sample i, augmented by the set of singular factors (principal components) of the 78 sample spectra, so that p=378; with singular factors indexed 301; : : : ; 378.

The analysis was developed repeatedly, exploring aspects of model fit and prediction of the validation sample as the number of control parameters were varied. The particular parameters of key interest varied were the Bayes' factor thresholds that define splits, and controls on the number of such splits that may be made at any one node. It was determined that across ranges of these control parameters, that there was a good degree of robustness. The Bayes' factor threshold was fixed at 3 on the log scale, after which and two-level trees were explored allowing at most 10 splits of the root node and then at most 4 splits of each of nodes 1 and 2. This allowed up to 160 trees, with this analysis generating 148 trees.

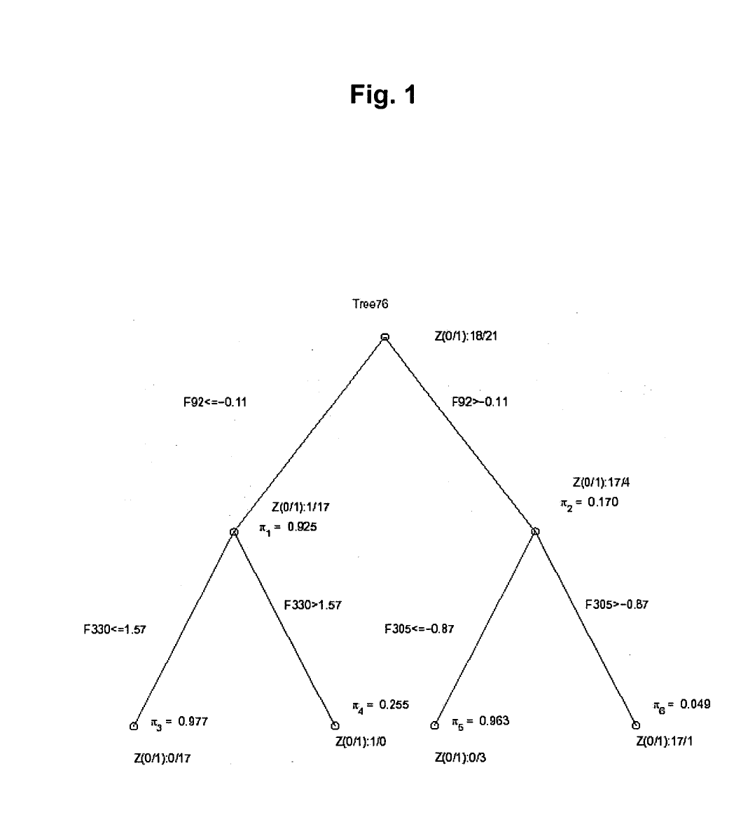

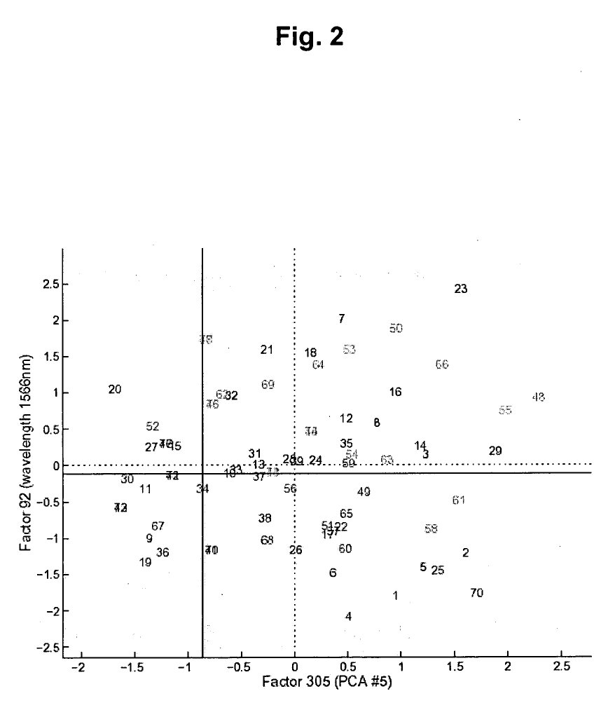

Many of the trees identified had one or two of the predictors in common, and represent variation in the threshold values for those predictors. FIGS. 1-3 display some summaries. FIG. 1 represents one of the 148 trees, split at the root node by the spectral predictor labeled factor 92 (corresponding to a wavelength of 1566 nm). Multiple wavelength values appear in the 148 trees, with values close to this appearing commonly, reflecting the underlying continuity of the spectra. The key second level predictor is factor 305, one of the principal component predictors. The data are scatter plotted on these two predictors in FIG. 2 with corresponding levels of the predictor-specific thresholds from this tree marked.

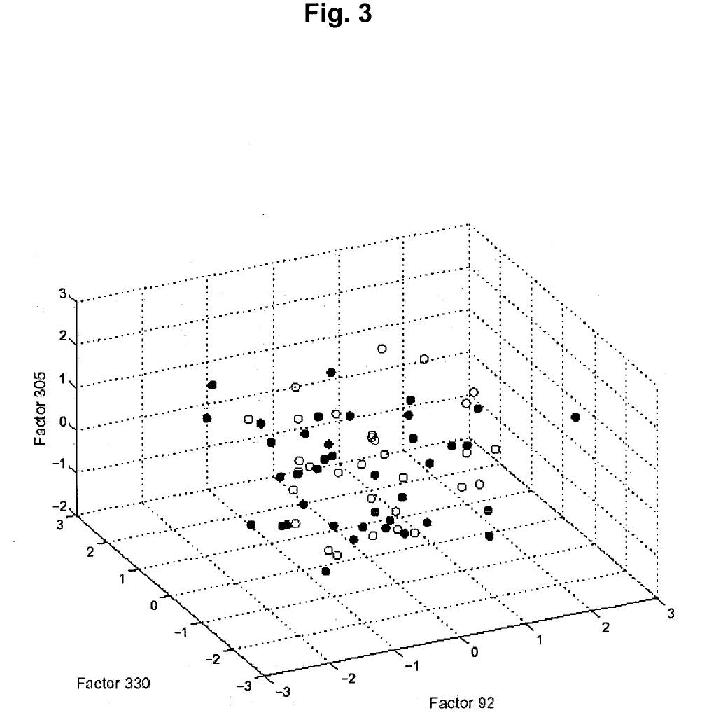

The data appears also against the three predictors in this tree in FIG. 3. Evidently there is substantial overlap in predictor space between the 0/1 outcomes, and cases close to the boundaries defined by any single tree are hard to accurately predict. Nevertheless, in terms of posterior predictive probabilities for the 39 validation samples, accuracy is good. By simply establishing the predictive probability threshold at 0.5 it is determined that 18 of 20 (90%) low fat (blue) cases are “correctly” predicted, as are 19 of 20 (95%) high fat (red) cases. Predictive accuracy is high in this example with considerable overlap between predictor patterns among the two outcome groups. This is a positive example of the use of the predictive tree approach in a context where standard methods, such as logistic regression, would be less useful. Furthermore, we end with a note that the 50:50 split of the 78 samples into training and validation sets followed the previous authors as references. Curious about this, we reran the analysis 500 times, each time randomly splitting the data 50:50 into training and validation samples. Predictive accuracy, as measured above, was generally not so good as reported for the initial sample split, varying from a little below 50% to 100% across this set of 500 analyses. The average accuracy for low fat (blue) cases was 80%, and that for high fat (red) cases 76%.

### EXAMPLE 2

### Metagene Expression Profiling to Predict Estrogen Receptor Status of Breast Cancer Tumors

This example illustrates not only predictive utility but also exploratory use of the tree analysis framework in exploring data structure. Here, the tree analysis is used to predict estrogen receptor (“ER”) status of breast tumors using gene expression data. Prior analyses of such data involved binary regression models which utilized Bayesian generalized shrinkage approaches to factor regression. Specifically, prior statistical models involved the use of probit linear regression linking principal components of selected subsets of genes to the binary (ER positive/negative) outcomes. See West, M., Blanchette, C., Dressman, H., Ishida, S., Spang, R., Zuzan, H., Marks, J. R. and Nevins, J. R. Utilization of gene expression profiles to predict the clinical status of human breast cancer. Proc. Natl. Acad. Sci., 98, 11462-11467 (2001). However, the tree model presents some distinct advantages over Bayesian linear regression models in the analysis of large non-linear data sets such as these.

Primary breast tumors from the Duke Breast Cancer SPORE frozen tissue bank were selected for this study on the basis of several criteria. Tumors were either positive for both the estrogen and progesterone receptors or negative for both receptors. Each tumor was diagnosed as invasive ductal carcinoma and was between 1.5 and 5 cm in maximal dimension. In each case, a diagnostic auxiliary lymph node dissection was performed. Each potential tumor was examined by hematoxylin/eosin staining and only those that were >60% tumor (on a per-cell basis), with few infiltrating lymphocytes or necrotic tissue, were carried on for RNA extraction. The final collection of tumors consisted of 13 estrogen receptor (ER)+ lymph node (LN)+ tumors, 12 ER LN+ tumors, 12 ER+ LN tumors, and 12 ER LN tumors

The RNA was derived from the tumors as follows: Approximately 30 mg of frozen breast tumor tissue was added to a chilled BioPulverizer H tube (Bio101) (Q-Biogene, La Jolla, Calif.). Lysis buffer from the Qiagen (Chatsworth, Calif.) RNeasy Mini kit was added, and the tissue was homogenized for 20 sec in a MiniBeadbeater (Biospec Products, Bartlesville, Okla.). Tubes were spun briefly to pellet the garnet mixture and reduce foam. The lysate was transferred to a new 1.5-ml tube by using a syringe and 21-gauge needle, followed by passage through the needle 10 times to shear genomic DNA. Total RNA was extracted by using the Qiagen RNeasy Mini kit. Two extractions were performed for each tumor, and total RNA was pooled at the end of the RNeasy protocol, followed by a precipitation step to reduce volume. Quality of the RNA was checked by visualization of the 28S:18S ribosomal RNA ratio on a 1% agarose gel. After the RNA preparation, the samples were subject to Affymetrix GENECHIP analysis.

Affymetrix GENECHIP Analysis: The targets for Affymetrix DNA microarray analysis were prepared according to the manufacturer's instructions. All assays used the human HuGeneFL GENECHIP microarray. Arrays were hybridized with the targets at 45° C. for 16 h and then washed and stained by using the GENECHIP Fluidics. DNA chips were scanned with the GENECHIP scanner, and signals obtained by the scanning were processed by GENECHIP Expression Analysis algorithm (version 3.2) (Affymetrix, Santa Clara, Calif.).

The same set of n=49 samples used in the binary regression analysis described in West et al (2001) is analyzed in this study, using predictors based on metagene summaries of the expression levels of many genes. Metagenes are useful aggregate, summary measures of gene expression profiles. The evaluation and summarization of large-scale gene expression data in terms of lower dimensional factors of some form is utilized for two main purposes: first, to reduce dimension from typically several thousand, or tens of thousands of genes to a more practical dimension; second, to identify multiple underlying “patterns” of variation across samples that small subsets of genes share, and that characterize the diversity of patterns evidenced in the full sample. Although, the analysis is conducive to the use of various factor model approaches known to those skilled in the art, a cluster-factor approach is used here to define empirical metagenes. This defines the predictor variables x utilized in the tree model. Metagenes may be obtained by combining clustering with empirical factor methods. The metagene summaries used in the ER example were based on the following steps:

Assume a sample of n profiles of p genes;

(1) Screen genes to reduce the number by eliminating genes that show limited variation across samples or that are evidently expressed at low levels that are not detectable at the resolution of the gene expression technology used to measure levels. This removes noise and reduces the dimension of the predictor variable;

(2) Cluster the genes using k_means, correlated-based clustering. Any standard statistical package may be used. This analysis uses the xcluster software created by Gavin Sherlock (http://genomewww. stanford.edu/sherlock/cluster.html). A large number of clusters are targeted so as to capture multiple, correlated patterns of variation across samples, and generally small numbers of genes within clusters;

(3) Extract the dominant singular factor (principal component) from each of the resulting clusters. Again, any standard statistical or numerical software package may be used for this; this analysis uses the efficient, reduced singular value decomposition function (“SVD”) in the Matlab software environment.

In the analysis of the ER data, the original data was developed using Affymetrix arrays with 7129 sequences, of which 7070 were used (following removal of Affymetrix controls from the data.). The expression estimates used were log2 values of the signal intensity measures computed using the dChip software for post-processing Affymetrix output data (See Li, C. and Wong, W. H. Model-based analysis of oligonucleotide arrays: Expression index computation and outlier detection. Proc. Natl. Acad. Sci., 98, 31-36 (2001). With a target of 500 clusters, the xcluster software implementing the correlation-based k_means clustering produced p=491 clusters.

The corresponding p metagenes were then evaluated as the dominant singular factors of each of these cluster, as referenced above. The data comprised 40 training samples and 9 validation cases. Among the latter, 3 were initial training samples that presented conflicting laboratory tests of the ER protein levels, so casting into question their actual ER status; these were therefore placed in the validation sample to be predicted, along with an initial 6 validation cases selected at random. These three cases are numbers 14, 31 and 33. The color coding in the graphs is based on the first laboratory test (immunohistochemistry). Additional samples of interest are cases 7, 8 and 11, cases for which the DNA microarray hybridizations were of poor quality, with the resulting data exhibiting major patterns of differences relative to the rest.

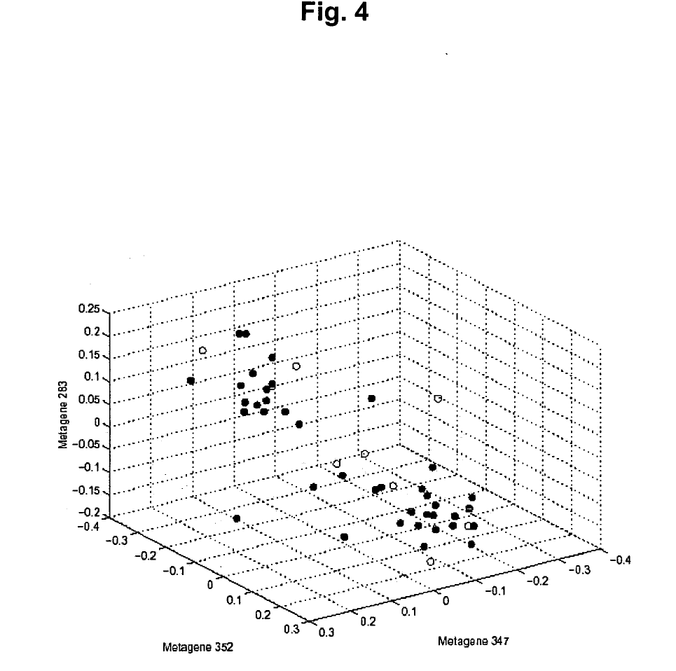

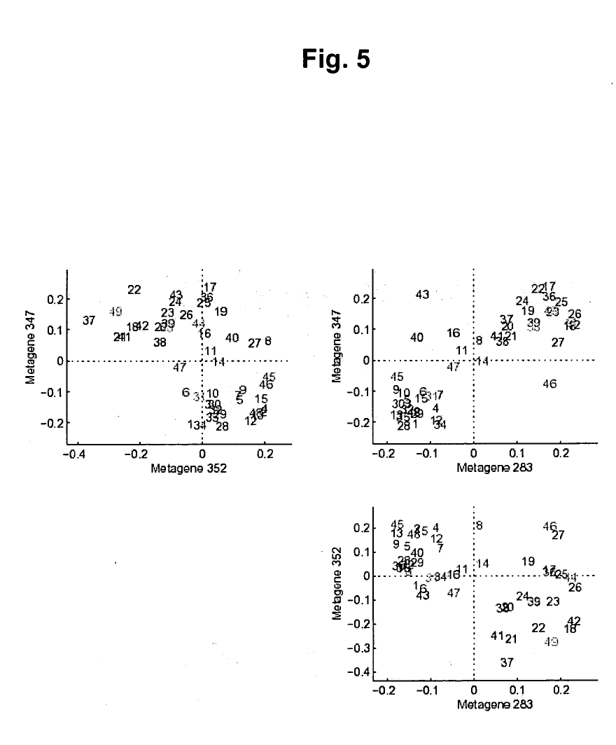

The metagene predictor has dimension p=491: the analysis generated trees based on a Bayes' factor threshold of 3 on the log scale, allowing up to 10 splits of the root node and then up to 4 at each of nodes 1 and 2. Some pertinent summaries appear in the following figures. FIGS. 4 and 5 display 3-D and pairwise 2-D scatterplots of three of the key metagenes, all clearly strongly related to the ER status and also correlated. However, there are in fact five or six metagenes that quite strongly associate with ER status and it is evident that they reflect multiple aspects of this major biological pathway in breast tumors. In the study reported in West et al (2001), Bayesian probit regression models were utilized with singular factor predictors which identified a single major factor predictive of ER. That analysis identified ER negative tumors 16, 40 and 43 as difficult to predict based on the gene expression factor model; the predictive probabilities of ER positive versus negative for these cases were near or above 0.5, with very high uncertainties reflecting real ambiguity.

In contrast to the more traditional regression models, the current tree model identifies several metagene patterns that together combine to define an ER profile of tumors, and that when displayed as in FIGS. 4 and 5 isolate these three cases as quite clearly consistent with their designated ER negative status in some aspects, yet conflicting and much more in agreement with the ER positive patterns on others. Metagene 347 is the dominant ER signature; the genes involved in defining this metagene include two representations of the ER gene, and several other genes that are coregulated with, or regulated by, the ER gene. Many of these genes appeared in the dominant factor in the regression prediction. This metagene strongly discriminates the ER 11 negatives from positives, with several samples in the mid-range.

Thus, it is no surprise that this metagene shows up as defining root node splits in many high-likelihood trees. This metagene also clearly defines these three cases—16, 40 and 43—as appropriately ER negative. However, a second ER associated metagene, number 352, also defines a significant discrimination. In this dimension, however, it is clear that the three cases in question are very evidently much more consistent with ER positives; a number of genes, including the ER regulated PS2 protein and androgen receptors, play roles in this metagene, as they did in the factor regression; it is this second genomic pattern that, when combined together with the first as is implicit in the factor regression model, breeds the conflicting information that fed through to ambivalent predictions with high uncertainty.

The tree model analysis here identifies multiple interacting patterns and allows easy access to displays such as those shown in FIGS. 4 to 6 that provide insights into the interactions, and hence to interpretation of individual cases. In the full tree analysis, predictions based on averaging multiple trees are in fact dominated by the root level splits on metagene 347, with all trees generated extending to two levels where additional metagenes define subsidiary branches. Due to the dominance of metagene 347, the three interesting cases noted above are perfectly in accord with ER negative status, and so are well predicted, even though they exhibit additional, subsidiary patterns of ER associated behavior identified in the figures.

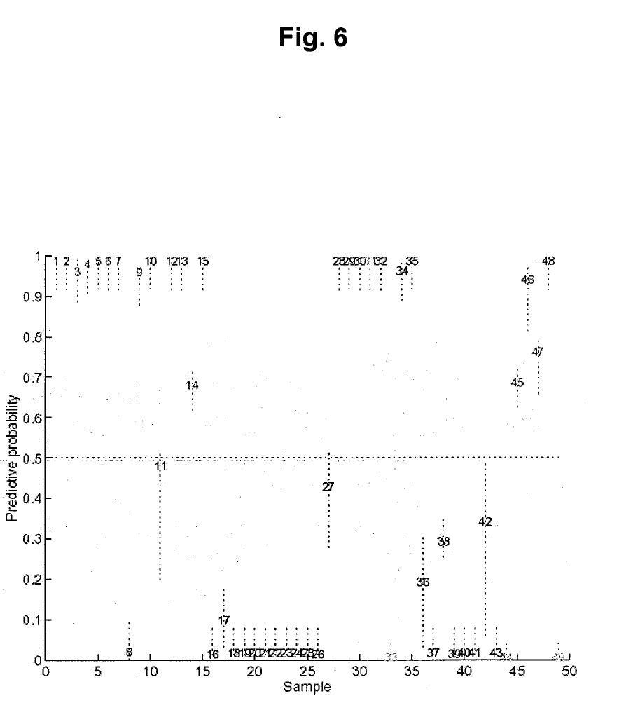

FIG. 6 displays summary predictions. The 9 validation cases are predicted based on the analysis of the full set of 40 training cases. Predictions are represented in terms of point predictions of ER positive status with accompanying, approximate 90% intervals from the average of multiple tree models. The training cases are each predicted in an honest, cross-validation sense: each tumor is removed from the data set, the tree model is then refitted completely to the remaining 39 training cases only, and the hold-out case is predicted, i.e., treated as a validation sample. Excellent predictive performance is observed for both these one-at-a-time honest predictions of training samples and for the out of sample predictions of the 9 validation cases. One ER negative, sample 31, is firmly predicted as having metagene expression patterns completely consistent with ER positive status. This is in fact one of the three cases for which the two laboratory tests conflicted. The other two such cases, however agree with the initial ER negative test result—number 33, for which the predictions firmly agree with the initial ER negative test result, and number 14, for which the predictions agree with the initial ER positive result though not quite so forcefully. The lack of conformity of expression patterns in some cases (Case 8, 11 and 7) are due to major distortions in the data on the DNA microarray due to hybridization problems.

### EXAMPLE 3

### Prediction of Lymph Node Metastases and Cancer Recurrence

This study assesses complex, multivariate patterns in gene expression data from primary breast tumor samples that can accurately predict nodal metastatic states and relapse for the individual patient using the statistical tree model of the invention.

DNA microarray data on samples of primary breast tumors was generated to which non-linear statistical analyses embodied by the tree model of the invention was applied to evaluate multiple patterns of interactions of groups of genes that have true predictive value, at the individual patient level, with respect to lymph node metastasis and cancer recurrence. For both lymph node metastasis and cancer recurrence, patterns of gene expression (metagenes) were identified that associate with outcome. Much more importantly, these patterns were capable of honestly predicting outcomes in individual patients with about 90% accuracy, based on a simple threshold of 0.5 probability in each case. The metagenes that predict lymph node metastasis and recurrence identify distinct groups of genes, suggesting different biological processes underlying these two characteristics of breast cancer.

Patients and biopsy specimens: The analyses of gene expression phenotypes drew samples from 171 primary tumor biopsies at the Koo Foundation Sun Yat-Sen Cancer Center (KF-SYSCC) in Taipei, Taiwan, collected and banked from 1991 to 2001. Samples from eleven patients who received preoperative chemotherapy and one with in-situ carcinoma were excluded from analysis. These 159 samples represent a heterogeneous population, though patient selection was enriched with cases of longer-term follow-up and observed recurrences. By September 2002, 62 patients developed recurrence whereas 97 remain disease free.

Microarray analysis: Tumor total RNA was extracted with Qiagen RNEasy kits, and assessed for quality with an Agilent Lab-on-a-Chip 2100 Bioanalyzer. Hybridization targets were prepared from total RNA according to Affymetrix protocols and hybridized to Affymetrix Human U95 GeneChip arrays See West M, Blanchette C, Dressman H, Huang E, Ishida S, Spang R et al. Predicting the clinical status of human breast cancer by using gene expression profiles, Proc Natl Acad Sci, 98:11462-11467 (2001).

Statistical analysis: This analysis again used the predictive statistical tree model. The method first screens genes to reduce noise, applies k-means correlation-based clustering targeting a large number of clusters, and then uses singular value decompositions (“SVD”) to extract the single dominant factor (principal component) from each cluster. This generated 496 cluster-derived singular factors (metagenes) that characterize multiple patterns of expression of the genes across samples. The strategy aimed to extract multiple such patterns while reducing dimension and smoothing out gene-specific noise through the aggregation within clusters. Formal predictive analysis then uses these metagenes in a Bayesian classification tree analysis. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. Iterative out-of-sample, cross-validation predictions are then performed leaving each tumor out of the data set one at a time, refitting the model from the remaining tumors and using it to predict the hold-out case. This rigorously tests the predictive value of a model and mirrors the real-world prognostic context where prediction of new cases as they arise is the major goal.

Although, clinico-pathologic parameters such as the presence or absence of positive auxiliary nodes represent the best means available to classify patients into broad subgroups by recurrence and survival, such methods remain an imperfect tool. Among patients with no detectable lymph node involvement, a population thought to be in a low risk category, between 22 and 33% develop recurrent disease after a 10-year follow-up. See Polychemotherapy for early breast cancer: an overview of the randomized trials, Early Breast Cancer Trialists' Collaborative Group, Lancet; 352:930-942 (2001). Thus, properly identifying individuals out of this group who are at risk for recurrence is beyond the current capabilities of most predictive diagnostics.

The question of lymph node diagnosis is part of the broader issue of more accurately predicting breast cancer disease course and recurrence. Recently, genomic-scale measures of gene expression, using microarrays and other technologies have opened a new avenue for cancer diagnosis. They identify patterns of gene activity that sub-classify tumors, and such patterns may correlate with the biological and clinical properties of the tumors. The utility of such data in improving prognosis will rely on analytical methods that accurately predict the behavior of the tumors based on expression patterns. Credible predictive evaluation is critical in establishing valid and reproducible results and implicating expression patterns that do indeed reflect underlying biology. This predictive perspective is a key step towards integrating complex data into the process of prognosis for the individual patient, a step that can be accomplished through the practice of the present invention.

Furthermore, an ultimate goal is to integrate molecular and genomic information with traditional clinical risk factors, including lymph node status, patient age, hormone receptor status, and tumor size, in comprehensive models for predicting disease outcomes. Rather than supplant traditional clinical appraisal, genomic data adds data to traditional risk factors, and assessing individuals based on combinations of relevant traditional risk factors with identified genomic factors could potentially improve predictions. The present invention allows this goal to be realized by demonstrating the ability of genomic data to accurately predict lymph node involvement and disease recurrence in defined patient subgroups. Most importantly, these predictions are relevant for the individual patient and can provide a quantitative measure of the probability for the clinical phenotype and outcome of disease. Such predictions may ultimately facilitate treating patients as individuals rather than as unidentifiable members of a risk profile.

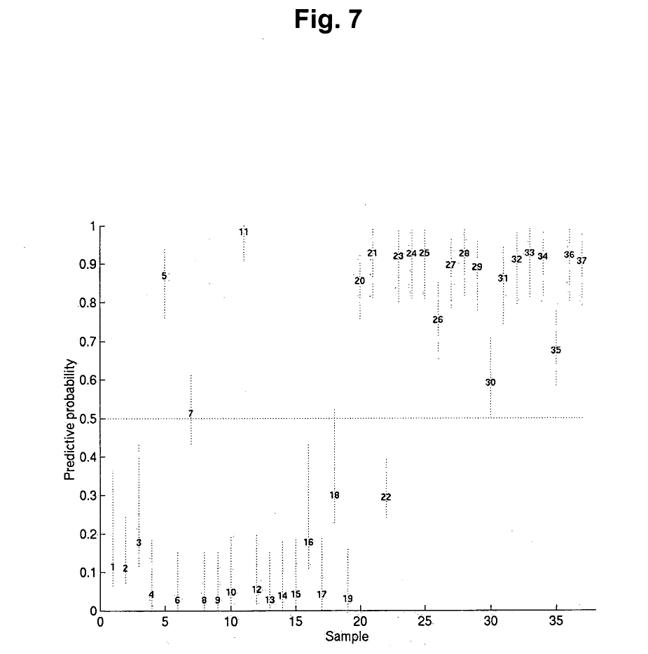

The binary prediction tree model was applied to the analysis of gene expression patterns in primary breast tumors that predict lymph node metastasis, as well as tumor recurrence. The first study compares traditional “low-risk” versus “high-risk” patients, primarily based on age, primary tumor size, lymph node status, and Estrogen receptor (“ER”) status. Among ER positive individuals, the “high-risk” clinical profile is represented by advanced lymph node metastases (10 or more positive nodes); the “low-risk profile” identifies node-negative women of age greater than 40 years with tumor size below 2 cm. The number of samples in the tumor collection that met these criteria reduced down to 18 high-risk and 19 low-risk cases. Expression data were generated and metagenes identified and used in the Bayesian statistical tree analysis. FIG. 7 displays summary predictions from the resulting total of 37 cross-validation analyses. For each individual tumor, this graph illustrates the predicted probability for “high-risk” versus “low-risk” (red versus blue) together with an approximate 90% confidence interval, based on analysis of the 36 remaining tumors performed successively 37 times as each tumor prediction is made. It is important to recognize that each sample in the data set, when assayed in this manner, constitutes a validation set that accurately assesses the robustness of the predictive model. The metagene model accurately predicts metastatic potential; about 90% of cases are accurately predicted based on a simple threshold at 0.5 on the estimated probability in each case. Case number 7 is in the intermediate zone, exhibiting patterns of expression of the selected metagenes that relate equally well to those of “high-” and “low-risk” cases, while case 22 is a clinical “high-risk” case with genomic expression patterns that relate more closely to “low-risk” cases. In contrast, node negative patients 5 and 11 have gene expression patterns more strongly indicative of “high-risk”, and are key cases for follow-up investigations.

Clinical features of these “discordant” cases are illuminating, and suggestive of how a broader investigation of clinical data combined with molecular model-based predictions may aid in the eventual decision-making process. Although case 22 did in fact recur, 6 years post-surgery; this patient's clinical classification as high risk for recurrence based on purely clinical parameters was moderated by a lower risk based on metagenes, as demonstrated by this patient having survived recurrence-free for a longer time. Thus the lower probability prediction assigned to patient 22 based on the gene expression profiles is reflected in the clinical behavior of her disease. The “low-risk” patient 7 recurred at 31 months, and patient 11 at 38 months, whereas case 5 is currently disease-free after only 12 months of follow-up. Again, case 7, and to some degree case 11, thus partly corroborate the predictions based on genomic criteria. data. With such predictions as part of a prognostic model, more intensive or innovative post-surgical therapy should perhaps have been recommended for these two cases.

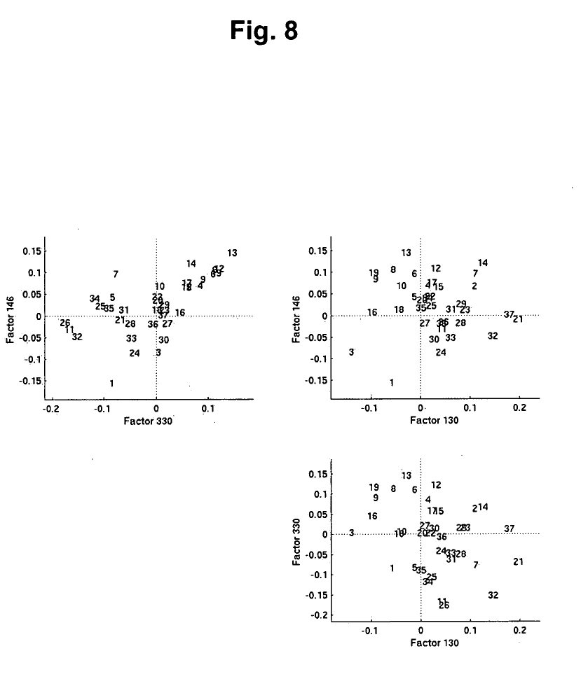

A critical aspect of the analyses described here is allowing the complexity of distinct gene expression patterns to enter the predictive model. Tumors are graphed against metagene levels for three of the highest scoring metagene factors as shown in FIG. 8. This analysis highlights the need to analyze multiple aspects of gene expression patterns. For example, if the low-risk cases 1, 3 and 11 are assessed against metagene 146 alone, their levels are more consistent with high-risk cases. However, when additional dimensions are considered, the picture changes. The second frame (upper right) shows that low-risk is consistent with low levels of metagene 130 or high levels of metagene 146; hence, cases 1 and 3 are not inconsistent in the overall pattern, though case 11 is consistent.

An analysis that selects one set of genes, summarized here as one metagene, as a “predictor” would be potentially misleading, as it ignores the broader picture of multiple interlocked genomic patterns that together characterize a state. In the predictions, these two metagenes play key roles: low levels of metagene 146 coupled with higher levels of metagene 130 are strongly predictive of high-risk cases. Combined use of multiple metagenes, in the context of the tree selection model building process, ultimately yields a pattern that has the capacity to accurately predict the clinical outcome.

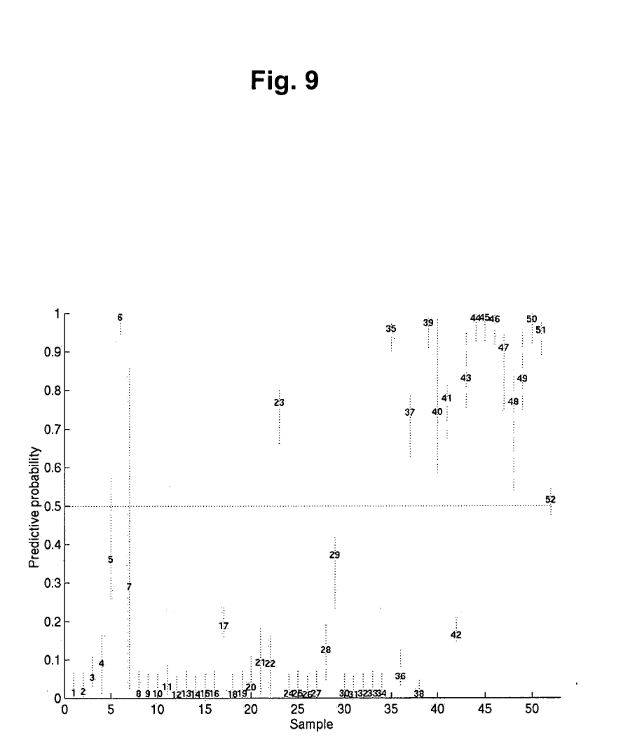

The second analysis concerns 3 year recurrence following primary surgery among the challenging and varied subset of patients with 1-3 positive lymph nodes. Such patients typically receive adjuvant chemotherapy alone, but more than 20% suffer relapse within five years. Hence, improved prognosis for this heterogeneous group is of critical importance; patients identified with a high probability of relapse could be targeted for more intensive treatment. The dataset provided 52 ER-positive cases in this lymph node category (34 non-recurrent, 18 recurrent). The aggregate predictions from the sets of generated statistical tree models defines a rather accurate picture; once again, there is an approximate 90% overall predictive accuracy in the 52 separate one-at-a-time, cross-validation prediction assessments as shown in FIG. 9. Based on the gene expression analysis, the 3 year non-recurrent cases 6 and 23, having profiles more akin to recurrent cases, would be candidates for intensive treatment. These patients did receive adjuvant chemotherapy based on additional clinical risk factors (especially tumor size). Thus traditional clinical risk factors other than lymph node status also indicate higher risk of recurrence for these two cases, consistent with the molecular predictions. Each actually survived recurrence-free for over three years; case 6 recurred at 42 months and case 23 remains disease-free after over 6 years. Cases with low genomic criteria for recurrence would be 36, 38 and 42. They, however, experienced recurrence within three years. These are cases that, under prognosis informed by only the genomic model, would have been indicated as more benign and not candidates for intensive treatment, whereas such a treatment might have proven to be more beneficial.

The tree model identified subsets of genes related to the metagene predictors of lymph node involvement. These are replete with those involved in cellular immunity, including a high proportion of genes that function in the interferon pathway. They include genes that are induced by interferon such as various chemokines and chemokine receptors (Rantes, CXCL10, CCR2), other interferon-induced genes (IFI30, IFI35, IFI27, IFIT1, IFIT4, IFITM3), as well as interferon effectors (2′-5′ oligoA synthetase), and genes encoding proteins mediating the induction of these genes in response to interferon (STAT1 and IRF1). This connection is intriguing given the role of interferon as a mediator of the anti-tumor response and, together with the fact that many genes involved in T cell function (TCRA, CD3D, IL2R, MHC) are also included within the group that predict lymph node metastasis. This may reflect the distinct nature of these tumors that have acquired a metastatic potential that elicits an anti-tumor response that is ultimately unsuccessful or an aberration of the normal anti-tumor response.

Genes implicated in recurrence prediction as identified by the tree model do not exhibit such a striking functional clustering but do include many examples previously associated with breast cancer. Moreover, this group of genes is clearly distinct set from those that predict lymph node involvement. They include genes associated with cell proliferation control, both cell cycle specific activities (CDKN2D, Cyclin F, E2F4, DNA primase, DNA ligase), more general cell growth and signaling activities (MK2, JAK3, MAPK81P, and EF1□), and a number of growth factor receptors and G-protein coupled receptors, some of which have been shown to facilitate breast tumor growth (EpoR). Possibly, the poor prognosis with respect to survival reflects a more vigorous proliferative capacity of the tumor.

Thus, the genes implicated in the prediction of lymph node metastasis and overall recurrence of disease, although clearly representing interrelated phenomena, nevertheless reflect the participation of distinct biological processes. The tree model is thus flexible in that regard as it only selects those metagenes that are most relevant to the prediction in hand. By contrast, traditional statistical testing perspectives that focus on significant differences at a population parameter level may say little of practical significance in terms of an individual patient's prognosis. Furthermore, the tree model takes into account the relevant multiple features of the complex patterns of gene expression, especially in a context such as breast cancer where multiple, interacting biological and environmental processes define physiological states, and individual dimensions provide only partial information. The tree model of the present invention assesses the complex, multivariate patterns in gene expression data from primary tumor biopsies, exploring the value of such patterns in predicting lymph node metastasis and relapse, two critically important aspects of breast cancer, at the individual patient level. The tree model identifies multivariate patterns of gene expression that, in this realistic context of substantial patient heterogeneity, deliver predictive accuracy of about 90%. The probabilistic models highlight cases where uncertainty is high, and generate subsets of implicated genes that relate to the biology of metastasis and tumor evolution.

To ascertain the success of the tree model, an out-of-sample predictive assessment via cross-validation is always conducted. Any selection of gene, metagene or clinical variables must be part of each cross-validation analysis. The results of such “feature selection” will vary each time a tumor is analyzed, and can dramatically impact on predictive accuracy. Analyses that select a set of predictors based on the entire dataset, including the individual to be predicted, in advance of predictive evaluation are inappropriate, and lead to misleadingly over-optimistic conclusions about predictive value. For breast cancer recurrence, the results provide evidence for gene expression profiles associated with recurrence in a homogeneous cohort of low risk patients. There are, however, several distinctions. First is the evaluation of models on the basis of accuracy in prediction at the individual level, with predictions made in formal probabilistic terms. Second, multiple, related and interacting biological patterns, here represented as separate and distinct metagenes, together represent a clinical state. Reducing high-dimensional genomic data to a single index may sacrifice opportunity for understanding complex interactions (see FIG. 2) that are truly predictive. Thirdly, applicants believe that the integration of molecular profiles with clinical risk factors—rather than the replacement of clinical data with molecular data—will define the major step towards personalized prognosis utilizing genomic data, hence the need for stratification using clinical variables.

The above gene expression analysis approach to the identification of atherosclerotic phenotype determinative genes may be combined with one or more additional selection protocols in a “multi-prong” gene selection approach for identifying genes associated with an atherosclerotic phenotype. Additional selection protocols that can be employed in conjunction with the subject selection protocol include: (1) selection protocols that identify all currently known genes that are associated with atherosclerosis (e.g., as determined by using existing biological and clinical databases, e.g., by performing a thorough review of the published literature concerning biological research on atherosclerosis mechanism and clinical research related to drugs that have shown a beneficial, or detrimental, effect on patients with atherosclerotic clinical manifestations); (2) genes that have been identified as associated with atherosclerosis using human genetic studies, e.g., genetic linkage analysis (for example, one analyzes the genome of individuals who have presented with premature coronary heart disease (CAD, hard manifestations of CAD before 45 for men and before 50 for women, such as myocardial infarction or bypass surgery), and their siblings and studies markers within the genome of these individuals that co-segregate with the disease process. The localization of such markers across the entire genome allows for identification of “hot spots” that contain 10-300 genes. These genes become candidates for further analysis); (3) genes that have been identified as associated with atherosclerosis using mouse genetic studies, e.g., using mouse models of human disease (Using established mouse models of atherosclerosis, such as ApoE knock-out mice, one searches for “modifiers” that alter the development of the disease process, either increase or reduce, that come into play upon changing the genetic background of the mice. The modifiers thus identified, or their human equivalents, in turn, become candidate genes for further studies on human atherosclerosis); (4) genes that have been identified as associated with atherosclerosis using epigenetic and methylation studies (It is know that with aging, gene expression can be altered, yet the mechanism(s) for such altered expression remains an enigma. Changes in methylation of CpG islands within the promoter region of a multitude of genes can result in altered transcription of such genes, and applicants have shown that such changes can occur in cardiovascular tissues with aging. Typically, methylation of the CpG island within the promoter of a gene results in silencing of this gene. Such changes in DNA methylation have been called “epigenetic” as they do not represent necessarily inherited changes. Applicants have been surveying the genome of human aortas for the presence of genes whose methylation is altered within atherosclerotic regions compared to normal aorta tissue. The technique that applicants have used for this survey is called restriction landmark genome scanning, or RLGS. Applicants have already identified two genes, nucleolin and monocarboxylate transporter 3 (MCT3) that are differently methylated between normal and diseased aorta tissues. These genes have become members of our pool of “candidates”); (6) Genes identified by our previous analysis using Bayesian analysis approach. Where the above expression analysis approach is combined with one or more additional approaches to identify genes that are atherosclerotic phenotype determinative genes, the initial genes identified using each disparate selection protocol may be combined into a single set for further use, as described below, using a number of different combination protocols. For example, each of the initially identified subsets may be additively combined to produce a master set of genes for further use. Alternatively, only the common genes of one or more subsets may be placed in the final set of genes for further use. For example, where one develops five initial subsets of genes using five different selection criteria, such as the specific criteria listed above, only those genes common to at least two or more, three or more, or four or more of the initial subsets, including all of the initial subsets, may be chosen for inclusion in the final set.

The resultant final or master set of genes may be used as a collection of atherosclerotic phenotype determinative genes as described above. In addition, such a set may be used as an initial set or “library” of candidate genes for further study to identify SNPs that cause or are otherwise associated with an atherosclerotic phenotype.

FIG. 6 provides a flow diagram showing a selection procedure as described above as it would be used to identify atherosclerotic phenotype determinative gene variants, e.g., SNPs, which are then used, either singly or in combination, in a variety of different applications, including the applications described above in connection with the specific atherosclerotic phenotype determinative genes identified herein.

While the above selection approach of the subject invention is described above in terms of the identification of atherosclerotic phenotype determinative genes, included within the scope of the invention is the use of the above approach to identify genes that are determinative of other phenotypes, including other disease phenotypes, such as cancer, etc.

The following examples are offered by way of illustration and not by way of limitation.

Experimental

I. Tissue/Sample Procurement for Gene Expression Analysis

A serious challenge at the inception of this study was to find human arterial material that would be suitable for study of various stages of atherosclerosis and concurrent gene expression profiling. Although the most straightforward approach to the analysis of disease tissue would be the collection of material from individuals who have either succumbed to heart disease or those who are undergoing a heart transplant, this has the significant disadvantage of utilizing tissue at the end-stage of disease. Many previous studies have demonstrated that atherosclerosis is a long-term process associated with aging, with development of disease preceding the development of overt signs of disease. Hence, it is likely that end-stage tissue would not reflect events associated with initiation and progression of disease, but instead molecular events that reflect response to injury and associated repair processes.

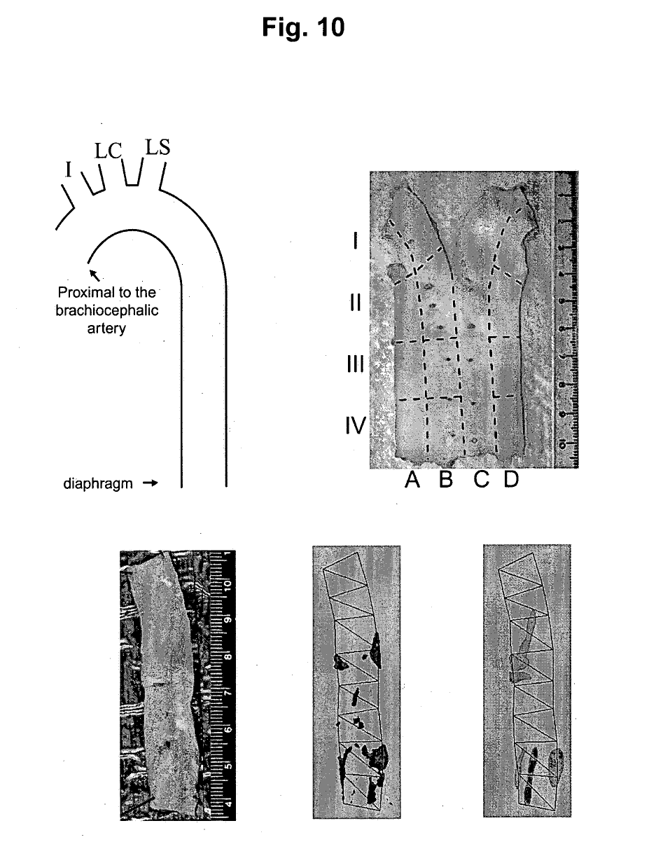

The aortas of heart donors were collected at organ harvest in University of Wisconsin solution on ice to minimize post-mortem changes. FIG. 10 shows the aorta sectioning. Strips A and B were frozen in liquid nitrogen for RNA extraction. Strip C was preserved in formalin for scoring atherosclerotic burden. The Pathobiological Determinants of Atherosclerosis in Youth study (PDAY) investigators showed that atherosclerosis displays increasing severity from proximal to distal locations. Also, they found sagittal symmetry with respect to disease burden across the longitudinal midline that approached 0.995. The mirroring of disease allowed us to ascribe the clinical atherosclerosis phenotype from one side to the gene expression pattern of the other. Early atherosclerotic plaques were assessed with imaging processing software by quantifying the area of Soudan IV staining. Advanced disease was quantified using PDAY methodologies to evaluate raised lesions 6. The data were expressed as a ratio of affected area over total surface of the studied section. Permission to procure these tissues was obtained from the North Carolina Organ Donor Services executive committee and approved by the institutional review board of the Duke University Medical Center.

II. RNA Preparation, Microarray Processing, Gene Expression Analysis

Techniques for microarray assays have been previously reported. See Huang E et al., Gene expression profiling for prediction of clinical characteristics of breast cancer. Recent Progress in Hormone Research, 2003; 55:55-73; West M, et al., Predicting the clinical status of human breast cancer by using gene expression profiles. Proc. Nat'l Acad. Sci. USA, 2001; 98:11462-11467. Briefly, aortic tissue was ground in liquid nitrogen. The RNA was extracted by the Trizol protocol and further purified with the Qiagen RNeasy kit. Quality was assessed with the Agilent Bioanalyzer and Affymetrix Test3 chips. The targets for microarray analysis were hybridized to U95Av2 Affymetrix microarrays and processed with the GENECHIP system. The signal intensity values were converted to a log2 scale following quantile normalization. Quantitative real-time PCR reactions were performed on 13 aorta samples and compared to microarray expression measures with a high degree of correlation across these genes. Replicate microarray assays were performed also showing a high degree of correlation in signal intensities (data not shown).

III. Design of Phenotyping Studies.

Applicants prioritized genes by their ability to predict two clinical phenotypes: disease extent and aorta location. Disease extent was scored by combining Sudan IV staining and raised lesion data. The “minimally diseased” group showed less than 5% Sudan IV staining and contained no raised lesions. The “severely diseased” group contained both raised lesions and extensive Sudan IV staining. Applicants analyzed sections from two identical locations in all the aortas, a proximal and distal section. From this pool, applicants identified 15 minimally diseased and 16 severely diseased sections for this analysis. Nine of these sections came from single aortas. The size of a particular section used in the analysis was quite small, on average 10 mm by 5 mm, making Sudanaphilia and raised lesion content homogeneous throughout the section.

The second phenotype was the location of the section within the thoracic aorta as a surrogate for disease susceptibility. This assumption is based on the conclusive evidence from the PDAY study that progression of disease advances from the distal to proximal areas of the aorta suggesting that distal regions are more susceptible to disease development. See Cornhill J F, et al. Topography of human aortic sudanophilic lesions. Monogr. Atheroscler. 1990; 15:13-19. As stated above, applicants analyzed sections from identical locations in all the aortas. There were 31 proximal (1A) sections and 32 distal (4B) sections in our analysis of aorta location. Applicants used the same pool of aorta sections for both analyses.

IV. Statistical Analysis.

Statistical analysis was performed utilizing the metagene construction and binary prediction tree analysis used previously in our analysis of gene expression patterns predictive of breast cancer outcomes. Huang E et al., Lancet. 2003; 361:1590-1596; Huang E. et al., Nature Genetics. 2003; 34:226-230. The initial step filtered out genes whose maximum expression did not exceed the median value of expression or did not vary more than 1-fold across the samples to remove genes with extremely low levels of expression or little variance. After the filter was applied, 7,470 of 12,563 total genes remained in the analysis.

Next, applicants clustered the genes into groups based on their expression patterns with the notion that related genes share similar variances in expression using k-means clustering. This algorithm randomly places genes into a predetermined number of groups. The genes are then shuffled among the groups in an iterative fashion to maximize the distinction between each groups. The number of designated clusters was also then varied iteratively to further maximize differences between the clusters. The resulting clusters contained anywhere from 20 to 50 genes and represented a unique gene expression pattern.

Singular value decomposition was performed on each cluster to generate a single factor, called a metagene. The metagene is the dominant expression pattern of a cluster and represents a group of genes that share a common gene expression signature in the context of a particular experimental condition. The metagenes are then used in binary decision trees to partition the samples into subgroups. In the trees, a metagene is used at a branch point to partition samples to one of two classifications based upon similarity or dissimilarity of a sample's gene expression pattern to the metagene. Each tree had several of these branches, and hundreds of trees were generated to determine the metagenes that did the best job of partitioning the samples. Within each metagene, applicants then identified the genes that lend the most weight to the dominant expression pattern.

To guard against over-fitting given the disproportionate number of variables to samples, applicants performed honest, out-of-sample cross validation analysis to test the stability and predictive capability of our model. Each aorta section was left out of the data set one at a time. The model was refitted (both the metagene factors and the partitions used) using the remaining samples, and the phenotype of the held out case was then predicted and the certainty of the classification is calculated.

Candidate gene annotation was performed using the Duke Integrated Genomics database.

V. Results

1. Assessment of Atherosclerosis Burden in Aorta Samples

Two analyses were performed: a comparison of minimally vs. severely diseased sections, and a comparison of proximal and distal sections of the thoracic aorta. The first analysis identified gene signatures associated with atherosclerotic severity. The two groups of aorta sections used were significantly different. There was significantly less Sudan IV staining (1.0%±0.0% vs. 19.3%±5.0%), and raised lesions (0.0%±0.0% vs. 42.4%±7.5%) in the minimally diseased samples, as shown in the following table:

| | | Severely | |
|---|---|---|---|
| | Minimally Diseased | Diseased | p value³ |
| Male Gender (%) | 50 | 50 | NS |
| Age mean | 38.2 | 55.6 | <0.0005 |
| (range) | (20-62) | (39-64) | |
| Sudan IV Staining¹ | 1.0% ± 0.0% | 19.3% ± 5.0% | <0.0005 |
| mean (±sem) | | | |
| Raised Lesion² | 0.0% ± 0.0% | 42.4% ± 7.5% | <0.000003 |
| mean (±sem) | | | |

¹Sudan IV Staining: percent of the total aorta that is stained.
²Raise Lesion: percent of the total aorta that contains raised lesions.
³Student T-test

The second analysis identified gene signatures associated with the proximal and distal locations within the thoracic aorta as a possible metric of atherosclerotic susceptibility. There were no significant difference in the characteristics of the two locations in either Sudan IV staining (12.4%±3.0% vs. 12.6%±3.1%) or raised lesions (10.1%±4.7% vs. 13.2%±5.3%). There was also no significant difference in the gender or ages of the donor pools an shown on the following table:

| | Low Susceptibility/ | High Susceptibility/ | |
|---|---|---|---|
| | 1A | 4B | p value³ |
| Male Gender (%) | 43.5 | 42.9 | NS |
| Age mean | 39.2 | 44.6 | 0.21 |
| (range) | (17-62) | (17-62) | |
| Sudan IV Staining¹ | 12.4% ± 3.0% | 12.6% ± 3.1% | 0.95 |
| mean (±sem) | | | |
| Raised Lesion² | 10.1% ± 4.7% | 13.2% ± 5.3% | 0.66 |
| mean (±sem) | | | |

¹Sudan IV Staining: percent of the total aorta that is stained.
²Raise Lesion: percent of the total aorta that contains raised lesions.
³Student T-test

2. Metagene Patterns Predictive of Extent of Atherosclerotic Lesions

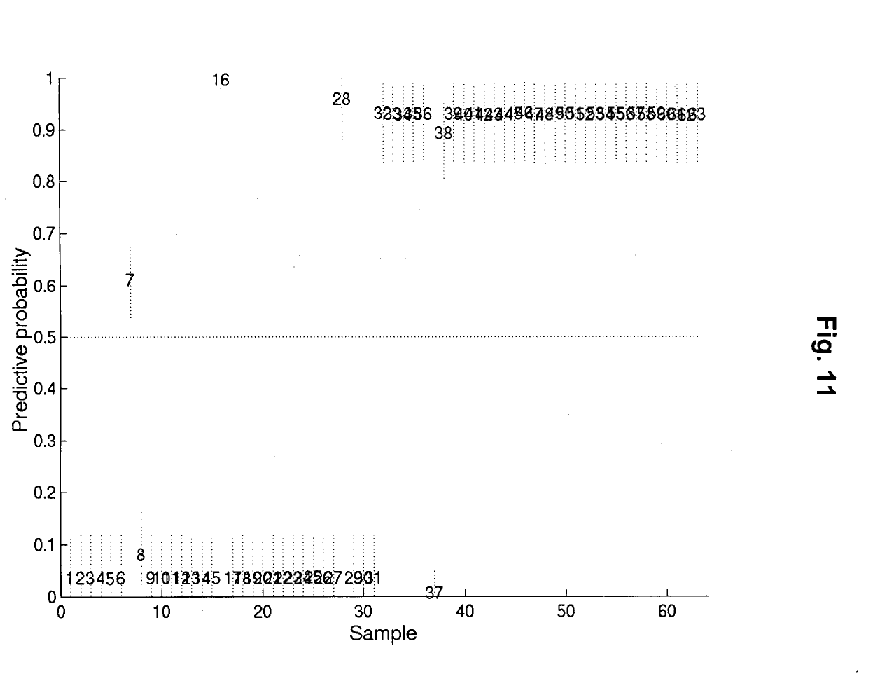

FIG. 11 displays the results from the analysis of disease severity where the predictive model correctly classifies 93.5% (29 of 31 sections) of the sections as minimally or severely diseased based solely upon their gene expression profiles. This figure shows results of the hold-one-out cross validation analysis where applicants construct the model from 30 samples and use it to predict the phenotype of the 31^{st} sample. The plot represents the probability that the unknown sample is severely diseased. The red numbers represent the severely diseased section with 95% confidence intervals; the blue numbers represent minimally diseased samples.

The gene prioritization process identified a set of 208 genes whose expression patterns provide the power to discriminate and predict disease states in our aorta samples as shown on Table I below which includes Genbank Deposit numbers and Unigene designations:

TABLE I
Genes associated with disease burden

| Genbank | Gene Symbol | Gene Name | UniGene |
|---|---|---|---|
| J04765 | SPP1 | secreted phosphoprotein 1 (osteopontin, bone | Hs.313 |
| | | sialoprotein I, early T-lymphocyte activation 1) | |
| AF052124 | SPP1 | secreted phosphoprotein 1 (osteopontin, bone | Hs.313 |
| | | sialoprotein I, early T-lymphocyte activation 1) | |
| X15525 | ACP2 | acid phosphatase 2, lysosomal | Hs.75589 |
| M94345 | CAPG | capping protein (actin filament), gelsolin-like | Hs.82422 |
| AB020687 | SLC21A9 | solute carrier family 21 (organic anion transporter), | Hs.7884 |
| | | member 9 | |
| U51240 | LAPTM5 | Lysosomal-associated multispanning membrane protein-5 | Hs.79356 |
| Y09445 | TBX5 | T-box 5 | Hs.50947 |
| AF053233 | VAMP8 | vesicle-associated membrane protein 8 (endobrevin) | Hs.172684 |
| U43185 | STAT5A | signal transducer and activator of transcription 5A | Hs.167503 |
| AL050008 | BRMS1 | breast cancer metastasis-suppressor 1 | Hs.100426 |
| M12529 | APOE | apolipoprotein E | Hs.169401 |
| AB022718 | DEPP | decidual protein induced by progesterone | Hs.93675 |
| Z24725 | MIG2 | mitogen inducible 2 | Hs.75260 |
| D13666 | OSF-2 | osteoblast specific factor 2 (fasciclin I-like) | Hs.136348 |
| U50928 | PKD2 | polycystic kidney disease 2 (autosomal dominant) | Hs.82001 |
| U46692 | CSTB | cystatin B (stefin B) | Hs.695 |
| AF044253 | KCNAB2 | potassium voltage-gated channel, shaker-related | Hs.298184 |
| | | subfamily, beta member 2 | |
| J03909 | IFI30 | interferon, gamma-inducible protein 30 | Hs.14623 |
| L10333 | RTN1 | reticulon 1 | Hs.99947 |
| M80634 | FGFR2 | fibroblast growth factor receptor 2 (bacteria-expressed | Hs.278581 |
| | | kinase, keratinocyte growth factor receptor, craniofacial | |
| | | dysostosis 1, Crouzon syndrome, Pfeiffer syndrome, | |
| | | Jackson-Weiss syndrome) | |
| M63138 | CTSD | cathepsin D (lysosomal aspartyl protease) | Hs.343475 |
| J04430 | ACP5 | acid phosphatase 5, tartrate resistant | Hs.1211 |
| AF044896 | ICB-1 | basement membrane-induced gene | Hs.10649 |
| X78565 | TNC | tenascin C (hexabrachion) | Hs.289114 |
| AB011143 | GAB2 | GRB2-associated binding protein 2 | Hs.30687 |
| X69819 | ICAM3 | intercellular adhesion molecule 3 | Hs.99995 |
| J02947 | SOD3 | superoxide dismutase 3, extracellular | Hs.2420 |
| U78095 | SPINT2 | serine protease inhibitor, Kunitz type, 2 | Hs.31439 |
| D67029 | SEC14L1 | SEC14-like 1 (S. cerevisiae) | Hs.75232 |
| AF013249 | LAIR1 | leukocyte-associated Ig-like receptor 1 | Hs.115808 |
| X62078 | GM2A | GM2 ganglioside activator protein | Hs.289082 |
| AB014574 | KIAA0674 | KIAA0674 protein | Hs.14799 |
| L13939 | AP1B1 | adaptor-related protein complex 1, beta 1 subunit | Hs.331602 |
| D90144 | SCYA3 | small inducible cytokine A3 | Hs.73817 |
| L06797 | CXCR4 | chemokine (C—X—C motif), receptor 4 (fusin) | Hs.89414 |
| D89077 | SLA | Src-like-adaptor | Hs.75367 |
| Y08374 | CHI3L1 | chitinase 3-like 1 (cartilage glycoprotein-39) | Hs.75184 |
| X02317 | SOD1 | superoxide dismutase 1, soluble (amyotrophic lateral | Hs.75428 |
| | | sclerosis 1 (adult)) | |
| AB002365 | KIAA0367 | KIAA0367 protein | Hs.23311 |
| AF084481 | WFS1 | Wolfram syndrome 1 (wolframin) | Hs.26077 |
| D34625 | TBXAS1 | thromboxane A synthase 1 (platelet, cytochrome P450, | Hs.2001 |
| | | subfamily V) | |
| AB011103 | KIF5C | kinesin family member 5C | Hs.6641 |
| AF041259 | ZNF217 | zinc finger protein 217 | Hs.155040 |
| J05037 | SDS | serine dehydratase | Hs.76751 |
| AF056087 | SFRP1 | secreted frizzled-related protein 1 | Hs.7306 |
| U81800 | SLC16A3 | solute carrier family 16 (monocarboxylic acid | Hs.85838 |
| | | transporters), member 3 | |
| AL050262 | TLR1 | toll-like receptor 1 | Hs.2474 |
| AB018271 | BPAG1 | bullous pemphigoid antigen 1 (230/240 kD) | Hs.198689 |
| AF014958 | CCRL2 | chemokine (C—C motif) receptor-like 2 | Hs.302043 |
| U58334 | TP53BP2 | tumor protein p53 binding protein, 2 | Hs.44585 |
| J03011 | IGLC6 | immunoglobulin lambda constant 6 (Kern+Oz− marker) | http://dig.cgt.duke.edu/cgi-bin/ |
| D12485 | ENPP1 | ectonucleotide pyrophosphatase/phosphodiesterase 1 | Hs.11951 |
| U22662 | NR1H3 | nuclear receptor subfamily 1, group H, member 3 | Hs.347353 |
| AF055008 | GRN | granulin | Hs.180577 |
| D86964 | DOCK2 | dedicator of cyto-kinesis 2 | Hs.17211 |
| U88629 | ELL2 | ELL-related RNA polymerase II, elongation factor | Hs.98124 |
| U75308 | TAF4 | TAF4 RNA polymerase II, TATA box binding protein | Hs.24644 |
| | | (TBP)-associated factor, 135 kD | |
| J03600 | ALOX5 | arachidonate 5-lipoxygenase | Hs.89499 |
| AF004709 | MAPK13 | mitogen-activated protein kinase 13 | Hs.178695 |
| L15388 | GPRK5 | G protein-coupled receptor kinase 5 | Hs.211569 |
| AB002361 | KIAA0363 | KIAA0363 protein | Hs.96633 |
| X62744 | HLA-DMA | major histocompatibility complex, class II, DM alpha | Hs.77522 |
| AF072099 | LILRB4 | leukocyte immunoglobulin-like receptor, subfamily B | Hs.67846 |
| | | (with TM and ITIM domains), member 4 | |
| X90858 | UP | uridine phosphorylase | Hs.77573 |
| Z29067 | NEK3 | NIMA (never in mitosis gene a)-related kinase 3 | Hs.2236 |
| U00952 | HPIP | hematopoietic PBX-interacting protein | Hs.8068 |
| J00194 | HLA-DRA | major histocompatibility complex, class II, DR alpha | Hs.76807 |
| M80254 | PPIF | peptidylprolyl isomerase F (cyclophilin F) | Hs.173125 |
| M21121 | SCYA5 | small inducible cytokine A5 (RANTES) | Hs.241392 |
| AF030339 | PLXNC1 | plexin C1 | Hs.286229 |
| AJ007395 | SIGLEC7 | sialic acid binding Ig-like lectin 7 | Hs.355493 |
| AF013570 | MYH11 | myosin, heavy polypeptide 11, smooth muscle | Hs.78344 |
| Z22555 | CD36L1 | CD36 antigen (collagen type I receptor, thrombospondin | Hs.180616 |
| | | receptor)-like 1 | |
| L22524 | MMP7 | matrix metalloproteinase 7 (matrilysin, uterine) | Hs.2256 |
| Y07512 | PRKG1 | protein kinase, cGMP-dependent, type I | Hs.2689 |
| Y00093 | ITGAX | integrin, alpha X (antigen CD11C (p150), alpha | Hs.51077 |
| | | polypeptide) | |
| AB007889 | KIAA0429 | KIAA0429 gene product | Hs.77694 |
| Y08136 | ASM3A | acid sphingomyelinase-like phosphodiesterase | Hs.42945 |
| AB014520 | KIAA0620 | KIAA0620 protein | Hs.301685 |
| L10678 | PFN2 | profilin 2 | Hs.91747 |
| Z98046 | MAGED2 | melanoma antigen, family D, 2 | Hs.4943 |
| D79994 | KIAA0172 | KIAA0172 protein | Hs.77546 |
| U29615 | CHIT1 | chitinase 1 (chitotriosidase) | Hs.91093 |
| D87074 | KIAA0237 | KIAA0237 gene product | Hs.78748 |
| X81109 | DXS1357E | accessory protein BAP31 | Hs.291904 |
| AL049946 | DKFZp564I1922 | adlican | Hs.72157 |
| U78556 | CRA | cisplatin resistance associated | Hs.166066 |
| U21931 | FBP1 | fructose-1,6-bisphosphatase 1 | Hs.574 |
| M63603 | PLN | phospholamban | Hs.85050 |
| D87433 | STAB1 | stabilin 1 | Hs.301989 |
| AB029032 | KIAA1109 | KIAA1109 protein | Hs.6606 |
| X12451 | CTSL | cathepsin L | Hs.78056 |
| U89606 | PDXK | pyridoxal (pyridoxine, vitamin B6) kinase | Hs.38041 |
| AB029018 | KIAA1095 | KIAA1095 protein | Hs.177635 |
| M34423 | GLB1 | galactosidase, beta 1 | Hs.79222 |
| AF095791 | TACC2 | transforming, acidic coiled-coil containing protein 2 | Hs.272023 |
| X03084 | C1QB | complement component 1, q subcomponent, beta | Hs.8986 |
| | | polypeptide | |
| X74039 | PLAUR | plasminogen activator, urokinase receptor | Hs.179657 |
| AB016811 | ARL7 | ADP-ribosylation factor-like 7 | Hs.111554 |
| X90976 | RUNX1 | runt-related transcription factor 1 (acute myeloid | Hs.129914 |
| | | leukemia 1; aml1 oncogene) | |
| U00802 | DBN1 | drebrin 1 | Hs.89434 |
| X96752 | HADHSC | L-3-hydroxyacyl-Coenzyme A dehydrogenase, short | Hs.8110 |
| | | chain | |
| Z49107 | LGALS9 | lectin, galactoside-binding, soluble, 9 (galectin 9) | Hs.81337 |
| AL080235 | RIS1 | Ras-induced senescence 1 | Hs.35861 |
| AF051851 | SVIL | supervillin | Hs.154567 |
| AF062075 | LPXN | leupaxin | Hs.49587 |
| AB000220 | SEMA3C | sema domain, immunoglobulin domain (Ig), short basic | Hs.171921 |
| | | domain, secreted, (semaphorin) 3C | |
| AF055581 | LNK | lymphocyte adaptor protein | Hs.13131 |
| AB015718 | STK10 | serine/threonine kinase 10 | Hs.16134 |
| X78817 | ARHGAP4 | Rho GTPase activating protein 4 | Hs.3109 |
| AJ000534 | SGCE | sarcoglycan, epsilon | Hs.110708 |
| M63835 | FCGR1A | Fc fragment of IgG, high affinity Ia, receptor for (CD64) | Hs.77424 |
| M16336 | CD2 | CD2 antigen (p50), sheep red blood cell receptor | Hs.89476 |
| M32578 | HLA-DRB1 | major histocompatibility complex, class II, DR beta 1 | Hs.375570 |
| U32324 | IL11RA | interleukin 11 receptor, alpha | Hs.64310 |
| AF079167 | OLR1 | oxidised low density lipoprotein (lectin-like) receptor 1 | Hs.77729 |
| M22324 | ANPEP | alanyl (membrane) aminopeptidase (aminopeptidase N, | Hs.1239 |
| | | aminopeptidase M, microsomal aminopeptidase, CD13, | |
| | | p150) | |
| D86961 | LHFPL2 | lipoma HMGIC fusion partner-like 2 | Hs.79299 |
| X54162 | LMOD1 | leiomodin 1 (smooth muscle) | Hs.79386 |
| U57911 | C11orf8 | chromosome 11 open reading frame 8 | Hs.46638 |
| M64571 | MAP4 | microtubule-associated protein 4 | Hs.239298 |
| AL023653 | CXorf9 | chromosome X open reading frame 9 | Hs.61469 |
| AL023653 | XPNPEP2 | X-prolyl aminopeptidase (aminopeptidase P) 2, | Hs.57922 |
| | | membrane-bound | |
| AC005546 | FLJ20244 | hypothetical protein FLJ20244 | Hs.158947 |
| AC005546 | LYL1 | lymphoblastic leukemia derived sequence 1 | Hs.46446 |
| M60028 | HLA-DQB1 | major histocompatibility complex, class II, DQ beta 1 | Hs.73931 |
| Y13622 | LTBP4 | latent transforming growth factor beta binding protein 4 | Hs.85087 |
| L76191 | IRAK1 | interleukin-1 receptor-associated kinase 1 | Hs.182018 |
| U83115 | AIM1 | absent in melanoma 1 | Hs.161002 |
| U60060 | FEZ1 | fasciculation and elongation protein zeta 1 (zygin I) | Hs.79226 |
| AJ011497 | CLDN7 | claudin 7 | Hs.278562 |
| D64142 | H1FX | H1 histone family, member X | Hs.109804 |
| D26350 | ITPR2 | inositol 1,4,5-triphosphate receptor, type 2 | Hs.238272 |
| X15414 | AKR1B1 | aldo-keto reductase family 1, member B1 (aldose | Hs.75313 |
| | | reductase) | |
| D87434 | KIAA0247 | KIAA0247 gene product | Hs.82426 |
| X79204 | SCA1 | spinocerebellar ataxia 1 (olivopontocerebellar ataxia 1, | Hs.74520 |
| | | autosomal dominant, ataxin 1) | |
| M58285 | HEM1 | hematopoietic protein 1 | Hs.132834 |
| AB014513 | LDB3 | LIM domain binding 3 | Hs.49998 |
| U63127 | TIC | SEC7 homolog | Hs.110121 |
| S59184 | RYK | RYK receptor-like tyrosine kinase | Hs.79350 |
| X53587 | ITGB4 | integrin, beta 4 | Hs.85266 |
| Z84718 | GSTT1 | glutathione S-transferase theta 1 | Hs.77490 |
| AF030409 | SLC9A6 | solute carrier family 9 (sodium/hydrogen exchanger), | Hs.62185 |
| | | isoform 6 | |
| J04621 | SDC2 | syndecan 2 (heparan sulfate proteoglycan 1, cell surface- | Hs.1501 |
| | | associated, fibroglycan) | |
| AF055024 | ASB1 | ankyrin repeat and SOCS box-containing 1 | Hs.153489 |
| U56833 | VBP1 | von Hippel-Lindau binding protein 1 | Hs.198307 |
| J05070 | MMP9 | matrix metalloproteinase 9 (gelatinase B, 92 kD | Hs.151738 |
| | | gelatinase, 92 kD type IV collagenase) | |
| AF093118 | FBLN5 | fibulin 5 | Hs.11494 |
| U12707 | WAS | Wiskott-Aldrich syndrome (eczema-thrombocytopenia) | Hs.2157 |
| M55531 | SLC2A5 | solute carrier family 2 (facilitated glucose/fructose | Hs.33084 |
| | | transporter), member 5 | |
| AB019527 | LDOC1 | leucine zipper, down-regulated in cancer 1 | Hs.45231 |
| X62055 | PTPN6 | protein tyrosine phosphatase, non-receptor type 6 | Hs.63489 |
| D83004 | UBE2N | ubiquitin-conjugating enzyme E2N (UBC13 homolog, | Hs.75355 |
| | | yeast) | |
| X76534 | GPNMB | glycoprotein (transmembrane) nmb | Hs.82226 |
| U45285 | TCIRG1 | T-cell, immune regulator 1, ATPase, H+ transporting, | Hs.46465 |
| | | lysosomal V0 protein a isoform 3 | |
| X63657 | FVT1 | follicular lymphoma variant translocation 1 | Hs.74050 |
| L09708 | C2 | complement component 2 | Hs.2253 |
| AB020316 | UST | uronyl-2-sulfotransferase | Hs.134015 |
| AF112219 | ESD | esterase D/formylglutathione hydrolase | Hs.82193 |
| Y14768 | BAT1 | HLA-B associated transcript 1 | Hs.55296 |
| Y14768 | NFKBIL1 | nuclear factor of kappa light polypeptide gene enhancer | Hs.2764 |
| | | in B-cells inhibitor-like 1 | |
| Y14768 | LTB | lymphotoxin beta (TNF superfamily, member 3) | Hs.890 |
| Y14768 | LTA | lymphotoxin alpha (TNF superfamily, member 1) | Hs.36 |
| Y14768 | ATP6V1G2 | ATPase, H+ transporting, lysosomal 13 kD, V1 subunit G | Hs.249227 |
| | | isoform 2 | |
| Y14768 | AIF1 | allograft inflammatory factor 1 | Hs.76364 |
| AL031846 | CBX7 | chromobox homolog 7 | http://dig.cgt.duke.edu/cgi-bin/ |
| AL031846 | DJ742C19.2 | phorbolin-1-related | Hs.226307 |
| AF036927 | ADCY9 | adenylate cyclase 9 | Hs.20196 |
| D49400 | ATP6V1F | ATPase, H+ transporting, lysosomal 14 kD, V1 subunit F | Hs.78089 |
| M55210 | LAMC1 | laminin, gamma 1 (formerly LAMB2) | Hs.214982 |
| U15085 | HLA-DMB | major histocompatibility complex, class II, DM beta | Hs.1162 |
| X97074 | AP2S1 | adaptor-related protein complex 2, sigma 1 subunit | Hs.119591 |
| D89016 | ARHGEF16 | Rho guanine exchange factor (GEF) 16 | Hs.87435 |
| AF022797 | KCNN4 | potassium intermediate/small conductance calcium- | Hs.10082 |
| | | activated channel, subfamily N, member 4 | |
| M33552 | LSP1 | lymphocyte-specific protein 1 | Hs.56729 |
| U09578 | MAPKAPK3 | mitogen-activated protein kinase-activated protein kinase 3 | Hs.227789 |
| M21186 | CYBA | cytochrome b-245, alpha polypeptide | Hs.68877 |
| M64925 | MPP1 | membrane protein, palmitoylated 1 (55 kD) | Hs.1861 |
| AF029750 | TAPBP | TAP binding protein (tapasin) | Hs.179600 |
| U10906 | CDKN1B | cyclin-dependent kinase inhibitor 1B (p27, Kip1) | Hs.238990 |
| U83993 | P2RX4 | purinergic receptor P2X, ligand-gated ion channel, 4 | Hs.321709 |
| AF022789 | USP12 | ubiquitin specific protease 12 | Hs.42400 |
| L35249 | ATP6V1B2 | ATPase, H+ transporting, lysosomal 56/58 kD, V1 | Hs.1697 |
| | | subunit B, isoform 2 | |
| M61916 | LAMB1 | laminin, beta 1 | Hs.82124 |
| AB011155 | DLG5 | discs, large (Drosophila) homolog 5 | Hs.170290 |
| X91809 | RGS19 | regulator of G-protein signaling 19 | Hs.22698 |
| U20158 | LCP2 | lymphocyte cytosolic protein 2 (SH2 domain-containing | Hs.2488 |
| | | leukocyte protein of 76 kD) | |
| S59049 | RGS1 | regulator of G-protein signaling 1 | Hs.75256 |
| U13991 | TAF10 | TAF10 RNA polymerase II, TATA box binding protein | Hs.89657 |
| | | (TBP)-associated factor, 30 kD | |
| X93498 | SH3BGR | SH3 domain binding glutamic acid-rich protein | Hs.47438 |
| M87770 | FGFR2 | fibroblast growth factor receptor 2 (bacteria-expressed | Hs.278581 |
| | | kinase, keratinocyte growth factor receptor, craniofacial | |
| | | dysostosis 1, Crouzon syndrome, Pfeiffer syndrome, | |
| | | Jackson-Weiss syndrome) | |
| AL050139 | FLJ13910 | hypothetical protein FLJ13910 | Hs.75277 |
| M73720 | CPA3 | carboxypeptidase A3 (mast cell) | Hs.646 |
| U35451 | CBX1 | chromobox homolog 1 (HP1 beta homolog Drosophila) | Hs.77254 |
| M32315 | TNFRSF1B | tumor necrosis factor receptor superfamily, member 1B | Hs.256278 |
| Y13710 | SCYA18 | small inducible cytokine subfamily A (Cys—Cys), | Hs.16530 |
| | | member 18, pulmonary and activation-regulated | |
| AB008109 | RGS5 | regulator of G-protein signaling 5 | Hs.24950 |
| M60830 | EVI2B | ecotropic viral integration site 2B | Hs.5509 |
| X71874 | PSMB10 | proteasome (prosome, macropain) subunit, beta type, 10 | Hs.9661 |
| AB007972 | PPP1R12B | protein phosphatase 1, regulatory (inhibitor) subunit 12B | Hs.130760 |
| X16663 | HCLS1 | hematopoietic cell-specific Lyn substrate 1 | Hs.14601 |
| M63193 | ECGF1 | endothelial cell growth factor 1 (platelet-derived) | Hs.73946 |
| D84110 | RBPMS | RNA-binding protein gene with multiple splicing | Hs.80248 |
| AJ006973 | TOM1 | target of myb1 (chicken) | Hs.9482 |
| AB002318 | TLN2 | talin 2 | Hs.150443 |
| U51333 | HK3 | hexokinase 3 (white cell) | Hs.159237 |
| U09577 | HYAL2 | hyaluronoglucosaminidase 2 | Hs.76873 |
| U00672 | IL10RA | interleukin 10 receptor, alpha | Hs.327 |

These genes in Table I encode proteins previously suspected to play a role in atherosclerosis including apolipoprotein E (apoE), osteopontin, and the oxidized LDL receptor 1 (olr1). Applicants performed a query against gene ontology databases to determine the important biological processes represented in the analysis. Applicants found that the genes reflected processes that applicants would infer from our current understanding of atherosclerosis such as cell cycle regulation and inflammatory response. Genes in these categories without direct links to atherosclerosis could be novel candidates for study. Such genes include capg, gm2 ganglioside activator protein, matrix metalloproteinase 9 (mmp9) and chemokine (C-C motif) receptor-like 2 (ccrl2).

Genes from table I were classified according to biological process as follows:

Apoptosis: accessory protein BAP31, dedicator of cyto-kinesis 2, hematopoietic protein 1, secreted frizzled-related protein 1, tumor necrosis factor receptor superfamily, member 1B, tumor protein p53 binding protein, 2.

Cell Adhesion: basement membrane-induced gene, bullous pemphigoid antigen 1, cathepsin D, claudin 7, fasciculation and elongation protein zeta 1, fibulin 5, glycoprotein, integrin, alpha X, intercellular adhesion molecule 3, laminin, beta 1, osteoblast specific factor 2, sialic acid binding Ig-like lectin 7, tenascin C.

Cell Motility: GRB2-associated binding protein 2, KIAA0429 gene product, lymphocyte-specific protein 1, myosin, heavy polypeptide 11, smooth muscle, plasminogen activator, urokinase receptor, profilin 2, talin 2

Cell Growth and Proliferation: cell proliferation, breast cancer metastasis-suppressor 1, follicular lymphoma variant translocation 1 Immune Response: accessory protein BAP31, capping protein, gelsolin-like, cathepsin L, CD2 antigen (p50), sheep red blood cell receptor, complement component 1, q subcomponent, complement component 2, dedicator of cyto-kinesis 2, immunoglobulin lambda constant 6, leukocyte immunoglobulin-like receptor, lymphocyte cytosolic protein 2, MHC, class II, DM alpha, MHC, class II, DM beta, MHC, class II, DQ beta 1, MHC, class II, DR alpha, MHC, class II, DR beta 1, osteopontin, TAP binding protein, Wiskott-Aldrich syndrome.

Inflammatory Response: allograft inflammatory factor 1, arachidonate 5-lipoxygenase, ATPase, H+ transporting, lysosomal 13 kD, carboxypeptidase A3, chemokine (C-X-C motif), receptor 4, granulin, HLA-B associated transcript 1, interleukin 10 receptor, alpha, interleukin 11 receptor, alpha, lymphotoxin alpha (TNF superfamily, member 1), lymphotoxin beta (TNF superfamily, member 3), small inducible cytokine A3, small inducible cytokine A5, small inducible cytokine subfamily A, m 18, thromboxane A synthase 1, tumor necrosis factor receptor superfamily, member 1B.

Chemotaxis: endothelial cell growth factor 1, lymphocyte-specific protein 1, plasminogen activator, urokinase receptor.

Cell Signaling: adenylate cyclase 9, ADP-ribosylation factor-like 7, chemokine (C-X-C motif), receptor 4, cyclin-dependent kinase inhibitor 1B, discs, large (Drosophila) homolog 5, fibroblast growth factor receptor 2, mitogen-activated protein kinase 13, mitogen-activated protein kinase-activated protein kinase 3, osteopontin, regulator of G-protein signaling 1, regulator of G-protein signaling 19, regulator of G-protein signaling 5, Rho GTPase activating protein 4, SH3 domain binding glutamic acid-rich protein, Src-like-adaptor, tumor protein p53 binding protein, 2.

Regulation of Transcription: chromobox homolog 1, endothelial cell growth factor 1, H1 histone family, member X, hematopoietic cell-specific Lyn substrate 1, integrin, beta 4, KIAA0363 protein, KIAA0363 protein, nuclear receptor subfamily 1 group H member 3, TAF10 RNA polymerase II, TATA box binding protein, TAF4 RNA polymerase II, TATA box binding protein, T-box 5, zinc finger protein 217.

Extracellular Matrix Structural Component: chitinase 1, hyaluronoglucosaminidase 2, laminin, beta 1, stabilin 1, syndecan 2.

Cytoskeleton/Structural Component: leiomodin 1, Lysosomal-associated multispanning membrane protein-5, reticulon 1, Rho GTPase activating protein 4, sarcoglycan, epsilon, solute carrier family 16, member 3, vesicle-associated membrane protein 8, Wolfram syndrome 1 Lipid Metabolism: apolipoprotein E, L-3-hydroxyacyl-Coenzyme A dehydrogenase, short chain.

Proteolysis: alanyl aminopeptidase, cathepsin L, matrix metalloproteinase 7, matrix metalloproteinase 9, proteasome subunit, beta type, 10, protein phosphatase 1, regulatory sub 12B Carbohydrate Metabolism: aldo-keto reductase family 1, member B1, fructose-1,6-bisphosphatase 1, galactosidase, beta 1, hexokinase 3, solute carrier family 2 member 5.

3. Metagene Patterns Predictive of Susceptibility to Atherosclerosis

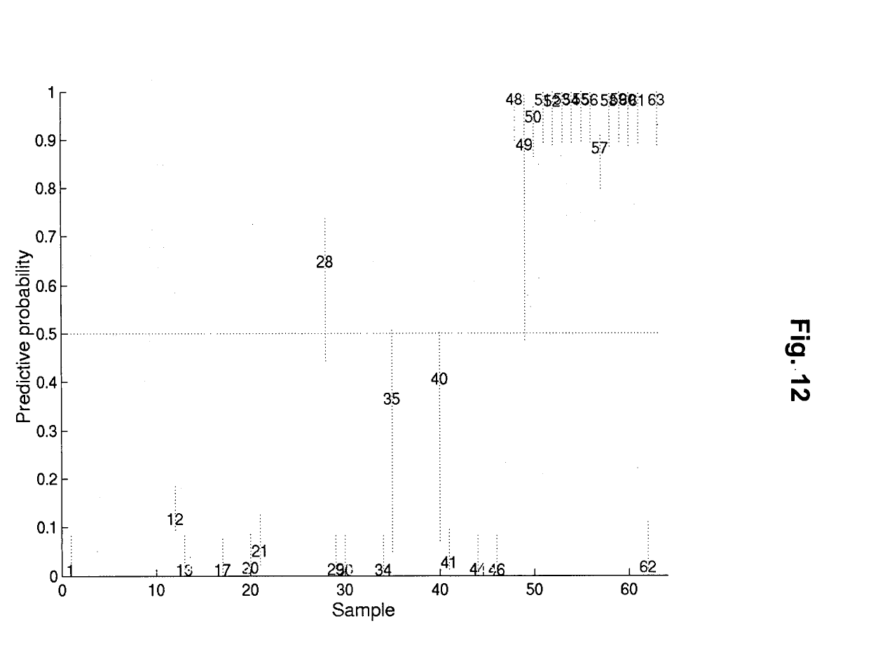

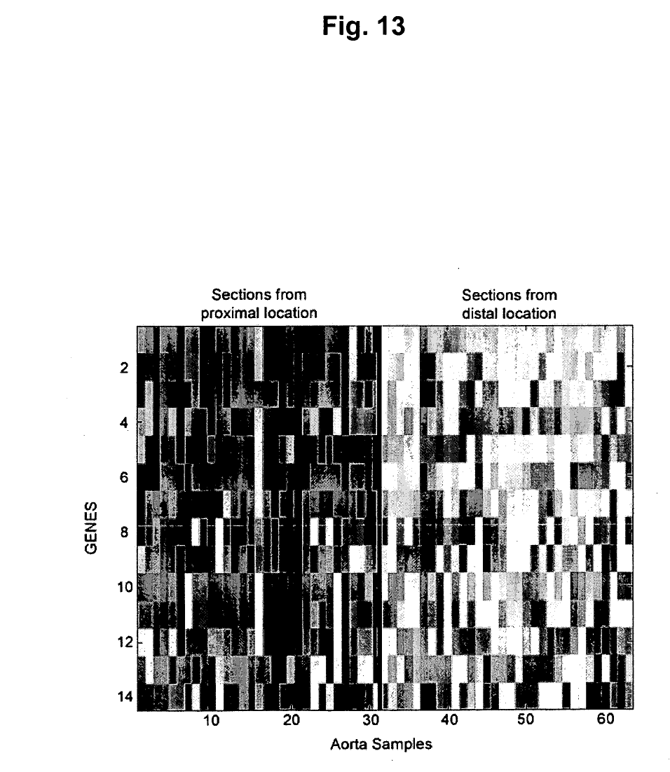

In our second analysis, applicants were able to predict the location of a sample within the thoracic aorta with 93.6% accuracy (59 of 63 sections correctly classified). The location may be a surrogate for disease susceptibility. FIG. 12 is a plot of the hold-one-out cross validation analysis that shows the probability that an unknown sample is from the distal aorta with 95% confidence intervals. The red numbers represent samples from the distal location; the blue numbers are from the proximal aorta. FIG. 13 shows expression levels by color display of the genes in the key predictive metagene and illustrates the differential expression patterns between proximal and distal tissues.

Twenty-five genes were identified that provided the predictive power in the analysis. These are listed in Table II, as follows:

TABLE II
Genes associated with disease susceptibility

| Genbank | Gene Symbol | Gene Name | UniGene |
|---|---|---|---|
| M26679 | HOXA5 | homeo box A5 | Hs.37034 |
| S82986 | HOXC6 | homeo box C6 | Hs.820 |
| AF051323 | SCAP2 | src family associated phosphoprotein 2 | Hs.52644 |
| J02947 | SOD3 | superoxide dismutase 3, extracellular | Hs.2420 |
| M16937 | HOXB7 | homeo box B7 | Hs.819 |
| K03000 | ALDH1A1 | aldehyde dehydrogenase 1 family, member A1 | Hs.76392 |
| M36711 | TFAP2A | transcription factor AP-2 alpha (activating enhancer binding | Hs.334334 |
| | | protein 2 alpha) | |
| D76435 | ZIC1 | Zic family member 1 (odd-paired homolog, Drosophila) | Hs.41154 |
| M74297 | HOXA4 | homeo box A4 | Hs.77637 |
| M68891 | GATA2 | GATA binding protein 2 | Hs.367725 |
| U43328 | CRTL1 | cartilage linking protein 1 | Hs.2799 |
| X17360 | HOXD4 | homeo box D4 | Hs.278255 |
| X51757 | HSPA6 | heat shock 70 kD protein 6 (HSP70B′) | Hs.3268 |
| U59831 | FOXD1 | forkhead box D1 | Hs.96028 |
| D83004 | UBE2N | ubiquitin-conjugating enzyme E2N (UBC13 homolog, yeast) | Hs.75355 |
| L49169 | FOSB | FBJ murine osteosarcoma viral oncogene homolog B | Hs.75678 |
| L35545 | PROCR | protein C receptor, endothelial (EPCR) | Hs.82353 |
| U16799 | ATP1B1 | ATPase, Na+/K+ transporting, beta 1 polypeptide | Hs.78629 |
| M20560 | ANXA3 | annexin A3 | Hs.1378 |
| X06256 | ITGA5 | integrin, alpha 5 (fibronectin receptor, alpha polypeptide) | Hs.149609 |
| Z22865 | DPT | dermatopontin | Hs.80552 |
| X75918 | NR4A2 | nuclear receptor subfamily 4, group A, member 2 | Hs.82120 |
| X16665 | HOXB2 | homeo box B2 | Hs.2733 |
| M97676 | MSX1 | msh homeo box homolog 1 (Drosophila) | Hs.1494 |
| M55153 | TGM2 | transglutaminase 2 (C polypeptide, protein-glutamine-gamma- | Hs.8265 |
| | | glutamyltransferase) | |

Some of the genes identified in this analysis, such as superoxide dismutase 3 (sod3) and protein C receptor (procr) have previously been associated with atherosclerosis. Interestingly, many genes that populate the dominant metagene have cellular roles that could be associated with atherosclerotic disease initiation such as homeobox-containing genes and gata2. An analysis of the biological processes represented by our gene list showed a preponderance of candidates relevant to regulation of transcription and signal transduction within our short list.

Genes from table II were classified according to biological process as follows:

Regulation of Cell Cycle: FBJ murine osteosarcoma viral oncogene homolog B.

Regulation of Transcription: nuclear receptor subfamily 4, group A, member 2, homeo box A4, msh homeo box homolog 1, transcription factor AP-2 alpha, forkhead box D1, homeo box B7, homeo box D4, homeo box C6.

Signal Transduction: src family associated phosphoprotein 2, Zic family member 1.

Inflammatory Response: protein C receptor, endothelial.

Cell Adhesion: Dermatopontin, cartilage linking protein 1, integrin alpha 5.

Development: homeo box B2.

From the aorta samples, 508 metagenes were identified. These are listed as follows:

Metagene 1; 35027_at Cluster Incl. X14830:Human mRNA for muscle acetylcholine receptor beta-; 34935_at Cluster Incl. AL021026:dJ127D3.3 (Flavin-containing Monooxygenase 2) /c; 37808_at Cluster Incl. AL049989:Homo sapiens mRNA; cDNA DKFZp564F052 (from clone; 39244_at Cluster Incl. M28211:Homo sapiens GTP-binding protein (RAB4) mRNA, comp; 39280_at Cluster Incl. U80744:Homo sapiens CTG4a mRNA, complete cds /cds=(387,81; 37604_at Cluster Incl. U44111:Human histamine N-methyltransferase (HNMT) gene /c; 32191_at Cluster Incl. D13969:Human mRNA for Mel-18 protein, complete cds /cds=(; 37364_at Cluster Incl. U72511:Human B-cell receptor associated protein (hBAP) mR; 37705_at Cluster Incl. U14575:Human (ard-1) mRNA, complete cds /cds=(935,1318) /; 38108_at Cluster Incl. AF020543:Homo sapiens palmitoyl-protein thioesterase-2 (P; 41579_s_at Cluster Incl. AI952267:wx50d11.x1 Homo sapiens cDNA, 3 end /clone=IM; 1624_at Stimulatory Gdp/Gtp Exchange Protein For C-Ki-Ras P21 And 5 mg P21; 673_at J04031 /FEATURE= /DEFINITION=HUMMDMCSF Human methylenetetrahydrofolate de.

Metagene 2; 39322_at Cluster Incl. AL109693:Homo sapiens mRNA full length insert cDNA clone; 34209_at Cluster Incl. D38169:Homo sapiens mRNA for inositol 1,4,5-trisphosphate; 37241_at Cluster Incl. AB020690:Homo sapiens mRNA for KIAA0883 protein, complete; 38832_r_at Cluster Incl. AF053356:Homo sapiens chromosome 7q22 sequence /cds=(25; 32587_at Cluster Incl. U07802:Human Tis11d gene, complete cds /cds=(291,1739) /g.

Metagene 3; 33586_at Cluster Incl. AF070620:Homo sapiens clone 24694 mRNA sequence /cds=UNKN; 37468_at Cluster Incl. AF058925:Homo sapiens Jak2 kinase mRNA, complete cds /cds; 38228_g_at Cluster Incl. AB006909:Homo sapiens mRNA for A-type microphthalmia as; 39295_s_at Cluster Incl. AF049884:Homo sapiens Arg/Abl-interacting protein ArgBP; 41431_at Cluster Incl. AB023153:Homo sapiens mRNA for KIAA0936 protein, complete; 41625_at Cluster Incl. AB011165:Homo sapiens mRNA for KIAA0593 protein, partial; 34668_at Cluster Incl. D88152:Homo sapiens mRNA for acetyl-coenzyme A transporte; 35139_at Cluster Incl. AL049341:Homo sapiens mRNA; cDNA DKFZp566A163 (from clone; 37951_at Cluster Incl. AF035119:Homo sapiens deleted in liver cancer-1 (DLC-1) m; 41727_at Cluster Incl. AB023224:Homo sapiens mRNA for KIAA1007 protein, partial; 34792_at Cluster Incl. AL049954:Homo sapiens mRNA; cDNA DKFZp564A1523 (from clon; 35764_at Cluster Incl. Y15164:Homo sapiens mRNA for protein encoded by cxorf5 (7; 37356_r_at Cluster Incl. D86326:Homo sapiens mRNA for p115, complete cds /cds=(1; 39474_s_at Cluster Incl. AF045800:Homo sapiens gremlin mRNA, complete cds /cds=(; 41498_at Cluster Incl. AB020718:Homo sapiens mRNA for KIAA0911 protein, complete.

Metagene 4; 36322_at Cluster Incl. AB012668:Homo sapiens hFuc-T VII gene for selectin-ligand; 38853_at Cluster Incl. X81892:H. sapiens mRNA for HE6 Tm7 receptor /cds=(72,3116); 40403_at Cluster Incl. Z97171:Homo sapiens GLC1A (trabecular meshwork induced g1; 32025_at Cluster Incl. Y11306:Homo sapiens mRNA for hTCF-4 /cds=(307,2097) /gb=Y; 33288_i_at Cluster Incl. D88827:Homo sapiens mRNA for zinc finger protein FPM315; 37183_at Cluster Incl. M81883:Human glutamate decarboxylase (GAD67) mRNA, comple; 39001_at Cluster Incl. AF047470:Homo sapiens malate dehydrogenase precursor (MDH; 35849_at Cluster Incl. AB011157:Homo sapiens mRNA for KIAA0585 protein, partial; 38804_at Cluster Incl. AF053641:Homo sapiens brain cellular apoptosis susceptibi; 1120_at J05459 /FEATURE=mRNA /DEFINITION=HUMGSTM3A Human glutathione transferase; 1121_g_at J05459 /FEATURE=mRNA /DEFINITION=HUMGSTM3A Human glutathione transfera; 1101_at L77864 /FEATURE= /DEFINITION=HUMFE65 Homo sapiens stat-like protein (Fe6. Metagene 5; 31386_at Cluster Incl. M20812:Human kappa-immunoglobulin germline pseudogene (co; 31396_r_at Cluster Incl. AB012851:Homo sapiens mRNA for Musashi, complete cds /c; 31410_at Cluster Incl. AF023614:Homo sapiens transmembrane activator and CAML in; 31590_g_at Cluster Incl. AL031983:dJ271M21.2 (hs6M1-12 (7 transmembrane receptor; 31594_at Cluster Incl. Y16788:Homo sapiens hHa3-I gene, complete CDS /cds=(0,121; 31626_i_at Cluster Incl. AF047485:Homo sapiens amine oxidase pseudogene mRNA, sp; 31681_at Cluster Incl. X57282:H. sapiens mRNA for soluble erythropoietin receptor; 32000_g_at Cluster Incl. AI344681:qp09h03.x1 Homo sapiens cDNA, 3 end /clone=IM; 32407_f_at Cluster Incl. U92818:Homo sapiens c33.28 unnamed HERV-H protein mRNA,; 32498_at Cluster Incl. L35318:Human rearranged metabotropic glutamate receptor t; 33016_at Cluster Incl. AI052224:oz21a02.x1 Homo sapiens cDNA, 3 end /clone=IMAG; 34066_at Cluster Incl. AJ012590:Homo sapiens mRNA for glucose 1-dehydrogenase /c; 34067_at Cluster Incl. AL022314:dJ1170K4.2 (novel Trypsin family protein with cl; 34089_at Cluster Incl. AB028953:Homo sapiens mRNA for KIAA1030 protein, partial; 34161_at Cluster Incl. U39573:Human salivary peroxidase mRNA, complete cds /cds=; 34573_at Cluster Incl. U14187:Human receptor tyrosine kinase ligand LERK-3 (EPLG; 35534_at Cluster Incl. AB011086:Homo sapiens mRNA for KIAA0514 protein, complete; 36332_at Cluster Incl. U40391:Human serotonin N-acetyltransferase gene, complete; 32866_at Cluster Incl. AB011177:Homo sapiens mRNA for KIAA0605 protein, complete; 34463_at Cluster Incl. M55983:Human DNase I mRNA, complete cds /cds=(159,1007) /; 34508_r_at Cluster Incl. AI971726:wr07a08.x1 Homo sapiens cDNA, 3 end /clone=IM; 34894_r_at Cluster Incl. AC003965:Homo sapiens chromosome 16, cosmid clone 325D7; 35939_s_at Cluster Incl. L20433:Human octamer binding transcription factor 1 (OT; 36222_at Cluster Incl. AJ012008:Homo sapiens genes encoding RNCC protein, DDAH p; 36276_at Cluster Incl. X67734:H. sapiens mRNA for transient axonal glycoprotein (; 36285_at Cluster Incl. U07364:Human inwardly rectifying potassium channel mRNA,; 36737_at Cluster Incl. U59057:Human beta-A4 crystallin (CRYBA4) mRNA, complete c; 37429_g_at Cluster Incl. AI140857:qa83c11.s1 Homo sapiens cDNA, 3 end /clone=IM; 37490_at Cluster Incl. L27213:Homo sapiens anion exchange protein mRNA, complete; 38851_at Cluster Incl. M63394:Human 3 nucleotide sequence mRNA /cds=UNKNOWN /gb=; 38903_at Cluster Incl. AF099731:Homo sapiens connexin 31.1 (GJB5) gene, complete; 38936_at Cluster Incl. M16652:Human pancreatic elastase IIA mRNA, complete cds /; 39308_r_at Cluster Incl. X81637:H. sapiens clathrin light chain b gene /cds=UNKNO; 39965_at Cluster Incl. AI570572:tm78c02.x1 Homo sapiens cDNA, 3 end /clone=IMAG; 40668_s_at Cluster Incl. U34624:Human T cell surface glycoprotein CD-6 mRNA, com; 41118_at Cluster Incl. AI921843:wp07a11.x1 Homo sapiens cDNA, 3 end /clone=IMAG; 41458_at Cluster Incl. AB007936:Homo sapiens mRNA for KIAA0467 protein, partial; 32100_r_at Cluster Incl. U06088:Human N-acetylgalactosamine 6-sulphatase (GALNS); 32699_s_at Cluster Incl. X64116:H. sapiens PVR gene for poliovirus receptor (exon; 32701_at Cluster Incl. U51269:Human armadillo repeat protein mRNA, complete cds; 33779_at Cluster Incl. AF060538:Homo sapiens vesicle associated membrane protein; 36004_at Cluster Incl. AF074382:Homo sapiens IkB kinase gamma subunit (IKK-gamma; 38622_at Cluster Incl. W28953:54b7 Homo sapiens cDNA /gb=W28953 /gi=1308901 /ug=; 38707_r_at Cluster Incl. S75174:E2F-4=transcription factor [human, Nalm6 and HeL; 34864_at Cluster Incl. AF070638:Homo sapiens clone 24448 unknown mRNA, partial c; 36114_r_at Cluster Incl. M19309:Human slow skeletal muscle troponin T mRNA, clon; 36663_at Cluster Incl. AL021155:dJ934G17.2.1 (Atrial Natriuretic Factor ANF prec; 38447_at Cluster Incl. U08438:Human beta-adrenergic receptor kinase (ADRBK1) gen; 39137_at Cluster Incl. X80878:H. sapiens R kappa B mRNA /cds=(130,4104) /gb=X8087; 39817_s_at Cluster Incl. AF040105:Homo sapiens RCL (Rcl) mRNA, complete cds /cds; 39832_at Cluster Incl. AL096723:Homo sapiens mRNA; cDNA DKFZp564H2023 (from clon; 39855_at Cluster Incl. AC005787:Homo sapiens chromosome 19, cosmid R33374 /cds=(; 39872_at Cluster Incl. AL031588:dJ1163J1.3 (novel protein similar to mouse B99); 39920_r_at Cluster Incl. AF095154:Homo sapiens C1q-related factor mRNA, complete; 41359_at Cluster Incl. Z98265:Homo sapiens mRNA for plakophilin 3 /cds=(74,2467); 41525_at Cluster Incl. N25429:yx76b02.s1 Homo sapiens cDNA, 3 end /clone=IMAGE-; 32557_at Cluster Incl. AI762438:wg57a07.x1 Homo sapiens cDNA, 3 end /clone=IMAG; 2079_s_at M13970 /FEATURE=mRNA /DEFINITION=HUMGFI21 Human insulin-like growth fa; 2046_at M21536 /FEATURE= /DEFINITION=HUMERG12 Human erg protein (ets-related gen; 2005_s_at U31317 /FEATURE= /DEFINITION=HSU31317 Human JAK family tyrosine protei; 1925_at Z36714 /FEATURE=mRNA /DEFINITION=HSCYCLF H. sapiens mRNA for cyclin F; 1894_f_at Neurofibromatosis 2 Tumor Suppressor; 1827_s_at M13929 /FEATURE=mRNA /DEFINITION=HUMMYCPOA Human c-myc-P64 mRNA, initi; 1494_f_at M33318 /FEATURE=mRNA /DEFINITION=HUMCPIIA3A Human cytochrome P450IIA3; 1432_s_at D16105 /FEATURE= /DEFINITION=HUMLTKLP2 Human mRNA for leukocyte tyrosi; 1244_at U18671 /FEATURE=mRNA /DEFINITION=HSU18671 Human Stat2 gene, complete cds; 1220_g_at X15949 /FEATURE=cds /DEFINITION=HSIRF2 Human mRNA for interferon regul; 1167_s_at D86331 /FEATURE= /DEFINITION=D86331 Human MT2-MMP gene for matrix meta; 1122_f_at K03183 /FEATURE=cds /DEFINITION=HUMCGBBA3 Human chorionic gonadotropin; 883_s_at M54915 /FEATURE= /DEFINITION=HUMPIM1LE Human h-pim-1 protein (h-pim-1); 789_at X52541 /FEATURE=cds /DEFINITION=HSEGR1 Human mRNA for early growth respon; 799_at X80343 /FEATURE=cds /DEFINITION=HSP35R H. sapiens p35 mRNA for regulatory; 493_at U29171 /FEATURE= /DEFINITION=HSU29171 Human casein kinase I delta mRNA, c; 421_at X66397 /FEATURE=cds /DEFINITION=HSTPRM H. sapiens tpr mRNA; 160020_at Z48481 /FEATURE=cds /DEFINITION=HSMMPM1 H. sapiens mRNA for membrane-ty.

Metagene 6; 38550_at Cluster Incl. AB021638:Homo sapiens X11L2 mRNA for X11-like protein 2,; 36108_at Cluster Incl. M16276:Human MHC class II HLA-DR2-Dw12 mRNA DQw1-beta, co; 37722_s_at Cluster Incl. U26266:Human deoxyhypusine synthase mRNA, complete cds; 1015_s_at U62293 /FEATURE=mRNA /DEFINITION=HSU62293 Human LIM-kinase1 and altern; 290_s_at L32831 /FEATURE=exon /DEFINITION=HUMGPCRD Homo sapiens G protein-couple.

Metagene 7; 31691_g_at Cluster Incl. U08997:Human glutamate dehydrogenase gene, complete cds; 34069_s_at Cluster Incl. S79325:SYT . . . SSX1 {translocation breakpoint} [human, sy; 32903_at Cluster Incl. L11695:Human activin receptor-like kinase (ALK-5) mRNA, c; 34925_at Cluster Incl. AB003062:Homo sapiens MYPT2 mRNA, complete cds /cds=(163,; 38207_at Cluster Incl. AW006742:wr28g10.x1 Homo sapiens cDNA, 3 end /clone=IMAG; 38230_at Cluster Incl. U81984:Human endothelial PAS domain protein 1 (EPAS1) mRN; 39225_at Cluster Incl. Y09443:H. sapiens mRNA for alkyl-dihydroxyacetonephosphate; 41373_s_at Cluster Incl. W27148:23g6 Homo sapiens cDNA /gb=W27148 /gi=1306727 /u; 31827_s_at Cluster Incl. AF027516:Homo sapiens trans-golgi network glycoprotein; 34217_at Cluster Incl. AB015132:Homo sapiens UKLF mRNA for ubiquitous Kruppel li; 34240_s_at Cluster Incl. AL049786:Novel human gene mapping to chomosome 13 /cds=; 34683_at Cluster Incl. U63289:Human RNA-binding protein CUG-BP/hNab50 (NAB50) mR; 37601_at Cluster Incl. AJ001417:Homo sapiens mRNA for extraneuronal monoamine tr; 38614_s_at Cluster Incl. U77413:Human O-linked GlcNAc transferase mRNA, complete; 40792_s_at Cluster Incl. AF091395:Homo sapiens Trio isoform mRNA, complete cds /; 32765_f_at Cluster Incl. W28330:45d4 Homo sapiens cDNA /gb=W28330 /gi=1308278 /u; 33417_at Cluster Incl. D31886:Human mRNA for KIAA0066 gene, partial cds /cds=(0,; 33855_at Cluster Incl. M96995:Homo sapiens epidermal growth factor receptor-bind; 36962_at Cluster Incl. U24105:Homo sapiens coatomer protein (COPA) mRNA, complet; 39523_at Cluster Incl. AF038897:Homo sapiens syntaxin 16 mRNA, complete cds /cds; 39531_at Cluster Incl. L06237:Human microtubule-associated protein 1B (MAP1B) ge; 40581_at Cluster Incl. U42390:Homo sapiens Trio mRNA, complete cds /cds=(66,8651; 40606_at Cluster Incl. U88629:Human RNA polymerase II elongation factor ELL2, co; 41287_s_at Cluster Incl. W28510:48f3 Homo sapiens cDNA /gb=W28510 /gi=1308521 /u; 2048_s_at M26747 /FEATURE= /DEFINITION=HUMCERBA Human c-erbA mRNA, complete cds; 1983_at X68452 /FEATURE=cds /DEFINITION=HSCYCD2 H. sapiens mRNA for cyclin D2; 1843_at Ret Transforming Gene; 1818_at Ras-Like Protein Tc10; 1793_at M80629 /FEATURE= /DEFINITION=HUMCHED Human cdc2-related protein kinase (; 1556_at U23946 /FEATURE= /DEFINITION=HSU23946 Human putative tumor suppressor (L; 1189_at X85753 /FEATURE= /DEFINITION=HSCDK8 Homo sapiens mRNA for CDK8 protein k; 976_s_at Z11695 /FEATURE=cds /DEFINITION=HS40KDAP H. sapiens 40 kDa protein kinas; 777_at D13988 /FEATURE= /DEFINITION=HUMRABGDI Human rab GDI mRNA, complete cds; 623_s_at M28213 /FEATURE= /DEFINITION=HUMRAB2A Homo sapiens GTP-binding protein; 591_s_at M33684 /FEATURE=cds /DEFINITION=HUMPPPB1A5 Human (clone lambda-16-1) no; 531_at U16307 /FEATURE= /DEFINITION=HSU16307 Human glioma pathogenesis-related p; 148_at U88629 /FEATURE=cds /DEFINITION=HSU88629 Human RNA polymerase II elongati.

Metagene 8; 31350_at Cluster Incl. AC004597:Homo sapiens chromosome 19, cosmid F20722 /cds=(; 35571_at Cluster Incl. AF055917:Homo sapiens protease-activated receptor 4 mRNA,; 35884_at Cluster Incl. Y07829:Homo sapiens RFB30 gene for RING finger protein /c; 40042_r_at Cluster Incl. U82381:Human proline dehydrogenase/proline oxidase (PRO; 31889_at Cluster Incl. U06452:Human melanoma antigen recognized by T-cells (MART; 32106_at Cluster Incl. L28101:Homo sapiens kallistatin (PI4) gene, exons 1-4, co; 35993_s_at Cluster Incl. AI698103:we20h11.x1 Homo sapiens cDNA, 3 end /clone=IM; 37977_at Cluster Incl. AI138834:qe04b02.x1 Homo sapiens cDNA, 3 end /clone=IMAG; 34872_at Cluster Incl. W26547:32h10 Homo sapiens cDNA /gb=W26547 /gi=1307390 /ug; 39187_at Cluster Incl. AF001450:untitled /cds=(0,1568) /gb=AF001450 /gi=2245523; 41340_at Cluster Incl. AA827795:od08a05.s1 Homo sapiens cDNA, 3 end /clone=IMAG; 695_at Tenascin.

Metagene 9; 36427_at Cluster Incl. W27129:22g8 Homo sapiens cDNA /gb=W27129 /gi=1306663 /ug=; 37106_at Cluster Incl. D89928:Homo sapiens HKL1 mRNA, complete cds /cds=(152,196; 38864_at Cluster Incl. W26851:17b12 Homo sapiens cDNA /gb=W26851 /gi=1306214 /ug; 39949_at Cluster Incl. AJ224328:Homo sapiens mRNA for MOCS1A & MOCS1B proteins,; 40665_at Cluster Incl. M83772:Human flavin-containing monooxygenase form II (FMO; 41372_at Cluster Incl. AB020638:Homo sapiens mRNA for KIAA0831 protein, complete; 31794_at Cluster Incl. D38524:Human mRNA for 5-nucleotidase /cds=(83,1768) /gb=D; 32039_at Cluster Incl. U81504:Homo sapiens beta-3A-adaptin subunit of the AP-3 c; 33260_at Cluster Incl. L13857:Human guanine nucleotide exchange factor mRNA, com; 33710_at Cluster Incl. U72515:Human C3f mRNA, complete cds /cds=(117,1262) /gb=U; 34201_at Cluster Incl. Y13350:Homo sapiens mRNA for DnaJ protein /cds=(0,975) /g; 35153_at Cluster Incl. AF058696:Homo sapiens cell cycle regulatory protein p95 (; 35184_at Cluster Incl. AB011118:Homo sapiens mRNA for KIAA0546 protein, partial; 35618_at Cluster Incl. D29677:Human mRNA for KIAA0054 gene, complete cds /cds=(1; 35681_r_at Cluster Incl. AB011141:Homo sapiens mRNA for KIAA0569 protein, comple; 36048_at Cluster Incl. AB015342:Homo sapiens HRIHFB2436 mRNA, partial cds /cds=(; 36057_at Cluster Incl. AB011084:Homo sapiens mRNA for KIAA0512 protein, complete; 36080_at Cluster Incl. AB002332:Human mRNA for KIAA0334 gene, complete cds /cds=; 36474_at Cluster Incl. AB018319:Homo sapiens mRNA for KIAA0776 protein, partial; 36521_at Cluster Incl. AB023213:Homo sapiens mRNA for KIAA0996 protein, complete; 36527_at Cluster Incl. AL050405:Novel human gene mapping to chomosome X /cds=(39; 36818_at Cluster Incl. AF052100:Homo sapiens clone 23645 mRNA sequence /cds=UNKN; 36849_at Cluster Incl. U90920:Human PTPL1-associated RhoGAP mRNA, complete cds /; 38252_s_at Cluster Incl. U84007:Human glycogen debranching enzyme isoform 1 (AGL; 38253_at Cluster Incl. U84011:Human glycogen debranching enzyme isoform 6 (AGL); 38270_at Cluster Incl. AF005043:Homo sapiens poly(ADP-ribose) glycohydrolase (hP; 38626_at Cluster Incl. AB007859:Homo sapiens KIAA0399 mRNA, partial cds /cds=(0,; 39379_at Cluster Incl. AL049397:Homo sapiens mRNA; cDNA DKFZp586C1019 (from clon; 39686_g_at Cluster Incl. AL050282:Homo sapiens mRNA; cDNA DKFZp586H2219 (from cl; 39706_at Cluster Incl. AB014536:Homo sapiens mRNA for KIAA0636 protein, complete; 39776_at Cluster Incl. AB014523:Homo sapiens mRNA for KIAA0623 protein, complete; 39785_at Cluster Incl. D42054:Human mRNA for KIAA0092 gene, complete cds /cds=(5; 40066_at Cluster Incl. AF046024:Homo sapiens UBA3 (UBA3) mRNA, complete cds /cds; 40129_at Cluster Incl. U47077:Human DNA-dependent protein kinase catalytic subun; 40404_s_at Cluster Incl. U18291:Human CDC16Hs mRNA, complete cds /cds=(24,1883); 40810_at Cluster Incl. U66615:Human SWI/SNF complex 155 KDa subunit (BAF155) mRN; 40844_at Cluster Incl. D63875:Human mRNA for KIAA0155 gene, complete cds /cds=(8; 41202_s_at Cluster Incl. AF000152:Homo sapiens OS-4 protein (OS-4) mRNA, complet; 33365_at Cluster Incl. AB023162:Homo sapiens mRNA for KIAA0945 protein, complete; 33870_at Cluster Incl. AB029005:Homo sapiens mRNA for KIAA1082 protein, partial; 33899_at Cluster Incl. U34252:Human gamma-aminobutyraldehyde dehydrogenase mRNA,; 34312_at Cluster Incl. AI040324:oy33a12.x1 Homo sapiens cDNA, 3 end /clone=IMAG; 34324_at Cluster Incl. AF068227:Homo sapiens putative transmembrane protein (CLN; 34327_at Cluster Incl. Z46606:H. sapiens HLTF gene for helicase-like transcriptio; 34825_at Cluster Incl. AL031775:dJ30M3.3 (novel protein similar to C. elegans Y6; 35289_at Cluster Incl. AJ011679:Homo sapiens mRNA for Rab6 GTPase activating pro; 35845_at Cluster Incl. AJ131245:Homo sapiens mRNA for Sec24 protein (Sec24β f; 36588_at Cluster Incl. AB018353:Homo sapiens mRNA for KIAA0810 protein, partial; 36596_r_at Cluster Incl. S68805:L-arginine-glycine amidinotransferase [human, ki; 36628_at Cluster Incl. L42542:Human RLIP76 protein mRNA, complete cds /cds=(223,; 37031_at Cluster Incl. D80005:Human mRNA for KIAA0183 gene, partial cds /cds=(0,; 37673_at Cluster Incl. X96586:H. sapiens mRNA for FAN protein /cds=(12,2765) /gb=; 38010_at Cluster Incl. AF002697:Homo sapiens E1B 19K/Bcl-2-binding protein Nip3; 38436_at Cluster Incl. D87440:Human mRNA for KIAA0252 gene, partial cds /cds=(0,; 38470_i_at Cluster Incl. D86981:Human mRNA for KIAA0228 gene, partial cds /cds=(; 38727_at Cluster Incl. M23161:Human transposon-like element mRNA /cds=UNKNOWN /g; 38763_at Cluster Incl. L29254:Human (clone P1-5) L-iditol-2 dehydrogenase gene /; 38843_at Cluster Incl. AL079310:Novel human gene mapping to chomosome 22 /cds=(5; 39823_at Cluster Incl. U06631:Human (H326) mRNA, complete cds /cds=(176,1969) /g; 40258_at Cluster Incl. M55265:Human casein kinase II alpha subunit mRNA, complet; 33113_at Cluster Incl. U65093:Human msg1-related gene 1 (mrg1) mRNA, complete cd; 2003_s_at U28946 /FEATURE= /DEFINITION=HSU28946 Human G/T mismatch binding prote; 1675_at M23379 /FEATURE= /DEFINITION=HUMGAPA Human GTPase-activating protein ras; 1213_at U88666 /FEATURE= /DEFINITION=HSU88666 Homo sapiens serine kinase SRPK2 m.

Metagene 10; 31669_s_at Cluster Incl. AF039307:Homo sapiens homeobox A11 (HOXA11) gene, compl; 31908_at Cluster Incl. U94354:Human lunatic fringe mRNA, partial cds /cds=(0,775; 33034_at Cluster Incl. Y17108:Homo sapiens mRNA for rhomboid-related protein, co; 33640_at Cluster Incl. Y14768:Homo sapiens DNA, cosmid clones TN62 and TN82 /cds; 35514_at Cluster Incl. AA916905:oh85a09.s1 Homo sapiens cDNA, 3 end /clone=IMAG; 33952_at Cluster Incl. U71601:Human zinc finger protein zfp47 (zf47) mRNA, parti; 34005_at Cluster Incl. X73079:Homo sapiens encoding Polymeric immunoglobulin rec; 34963_at Cluster Incl. AA557228:nl75c09.s1 Homo sapiens cDNA, 3 end /clone=IMAG; 35387_r_at Cluster Incl. S71129:acetylcholinesterase {14-E5 doman} [human, tumor; 35492_at Cluster Incl. AC004523:Homo sapiens chromosome 19, cosmid F22329 /cds=(; 38229_at Cluster Incl. X90579:H. sapiens DNA for cyp related pseudogene /cds=UNKN; 38512_r_at Cluster Incl. D26158:Homo sapiens mRNA for PLE21 protein, complete cd; 38528_at Cluster Incl. U19822:Human acetyl-CoA carboxylase mRNA, complete cds /c; 38566_at Cluster Incl. X60382:H. sapiens COL10A1 gene for collagen (alpha-1 type; 38607_at Cluster Incl. AF027204:Homo sapiens putative tetraspan transmembrane pr; 39242_at Cluster Incl. X96783:H. sapiens Syt V gene (genomic and cDNA sequence) /; 39660_at Cluster Incl. AI309115:qo71a09.x1 Homo sapiens cDNA, 3 end /clone=IMAG; 40003_at Cluster Incl. U36221:Human pancreatic zymogen granule membrane protein; 40288_r_at Cluster Incl. AA471042:ne23b10.s1 Homo sapiens cDNA, 3 end /clone=IM; 40337_at Cluster Incl. M35531:Human GDP-L-fucose-beta-D-galactoside 2-alpha-1-fu; 41036_at Cluster Incl. AL022314:dJ1170K4.1 (novel protein similar to KIAA0176 an; 41374_at Cluster Incl. AB016869:Homo sapiens mRNA for p70 ribosomal S6 kinase be; 41694_at Cluster Incl. M17754:Human BN51 mRNA, complete cds /cds=(51,1238) /gb=M; 33778_at Cluster Incl. AL096779:Novel human gene mapping to chomosome 2213.3 si; 35988_i_at Cluster Incl. AI417075:tg78e09.x1 Homo sapiens cDNA, 3 end /clone=IM; 36473_at Cluster Incl. AB023220:Homo sapiens mRNA for KIAA1003 protein, complete; 37210_at Cluster Incl. S78296:neurofilament-66 [human, fetal brain, mRNA, 3197 n; 37551_at Cluster Incl. D86966:Human mRNA for KIAA0211 gene, complete cds /cds=(5; 39803_s_at Cluster Incl. U84570:Human A2 mRNA, complete cds /cds=(239,883) /gb=U; 40094_r_at Cluster Incl. X80026:H. sapiens B-cam mRNA /cds=(6,1772) /gb=X80026 /g; 40842_at Cluster Incl. M60784:Human U1 snRNP-specific protein A gene /cds=(137,9; 39455_r_at Cluster Incl. AJ002607:Homo sapiens HOX11L1 gene, exon 1 and joined C; 39489_g_at Cluster Incl. W27720:39e11 Homo sapiens cDNA /gb=W27720 /gi=1307866 /; 39917_at Cluster Incl. AI961040:wq58f11.x1 Homo sapiens cDNA, 3 end /clone=IMAG; 41792_at Cluster Incl. L78207:Homo sapiens sulfonylurea receptor (SUR1) mRNA, co; 2028_s_at M96577 /FEATURE= /DEFINITION=HUME2F Homo sapiens (E2F-1) pRB-binding p; 1559_at U24153 /FEATURE= /DEFINITION=HSU24153 Human p21-activated protein kinase; 1280_i_at Serine/Threonine Kinase; 1169_at D88799 /FEATURE= /DEFINITION=D88799 Homo sapiens mRNA for cadherin, part; 970_r_at X98296 /FEATURE=cds /DEFINITION=HSUBIQHYD H. sapiens mRNA for ubiquitin; 934_at L11702 /FEATURE= /DEFINITION=HUMPHOSPDP Human phospholipase D mRNA, compl; 726_f_at Chorionic Somatomammotropin Hormone Cs-5; 729_i_at Mucin 3, Intestinal; 730_r_at Mucin 3, Intestinal; 519_g_at U07132 /FEATURE= /DEFINITION=HSU07132 Human steroid hormone receptor Ne; 255_s_at M13981 /FEATURE= /DEFINITION=HUMINHA Human inhibin A-subunit mRNA, comp.

Metagene 11; 32395_r_at Cluster Incl. X55954:Human mRNA for HL23 ribosomal protein homologue; 38922_at Cluster Incl. AF097738:Homo sapiens non-receptor tyosine kinase (TNK1); 39591_s_at Cluster Incl. Z36531:H. sapiens mRNA for fibrinogen-like protein (pT49; 39593_at Cluster Incl. AI432401:tg73b09.x1 Homo sapiens cDNA, 3 end /clone=IMAG; 41102_at Cluster Incl. U66359:Human T54 protein (T54) mRNA, complete cds /cds=(6; 31852_at Cluster Incl. AL050390:Homo sapiens mRNA; cDNA DKFZp564O043 (from clone; 33218_at Cluster Incl. M11730:Human tyrosine kinase-type receptor (HER2) mRNA, c; 37692_at Cluster Incl. AI557240:PT2.1_{—}15_C11.r Homo sapiens cDNA, 3 end /clone_{—}; 40269_at Cluster Incl. U51990:Human hprp18 mRNA, complete cds /cds=(72,1100) /gb; 32602_at Cluster Incl. X63465:H. sapiens hGDS mRNA for smg GDS /cds=(0,1676) /gb=; 213_at M97675 /FEATURE= /DEFINITION=HUMROR1A Human transmembrane receptor (ror1).

Metagene 12; 32635_at Cluster Incl. AB029036:Homo sapiens mRNA for KIAA1113 protein, partial; 38640_at Cluster Incl. AI582831:tn36c06.x1 Homo sapiens cDNA, 3 end /clone=IMAG; 40471_at Cluster Incl. Y09048:H. sapiens PxF gene /cds=(10,909) /gb=Y09048 /gi=25; 40822_at Cluster Incl. L41067:Homo sapiens NF-AT4c mRNA, complete cds /cds=(210,; 33364_at Cluster Incl. U51694:HSU51694 Homo sapiens cDNA /gb=U51694 /gi=1255286; 36978_at Cluster Incl. D38521:Human mRNA for KIAA0077 gene, partial cds /cds=(0,; 39546_s_at Cluster Incl. AA191426:zp83g09.s1 Homo sapiens cDNA, 3 end /clone=IM; 40273_at Cluster Incl. AA485440:zx90g03.s1 Homo sapiens cDNA, 3 end /clone=IMAG; 40895_g_at Cluster Incl. AI393342:tg44c04.x1 Homo sapiens cDNA, 3 end /clone=IM; 41506_at Cluster Incl. AF032437:Homo sapiens mitogen activated protein kinase ac.

Metagene 13; 31427_at Cluster Incl. U43604:Human unidentified mRNA, partial sequence /cds=UNK; 35547_at Cluster Incl. AF058056:Homo sapiens monocarboxylate transporter 2 (hMCT; 33562_g_at Cluster Incl. X80031:Homo sapiens COL4A3 mRNA /cds=(161,5173) /gb=X80; 35436_at Cluster Incl. L06147:Human (clone SY11) golgin-95 mRNA, complete cds /c; 35439_at Cluster Incl. D26121:Human mRNA for ZFM1 protein alternatively spliced; 35933_f_at Cluster Incl. D38498:Human PMS5 mRNA (yeast mismatch repair gene PMS1; 35958_at Cluster Incl. AL050379:Homo sapiens mRNA; cDNA DKFZp586F1922 (from clon; 37491_at Cluster Incl. D90359:Human CCG1 mRNA /cds=(51,5669) /gb=D90359 /gi=5593; 38176_at Cluster Incl. AF017656:Homo sapiens G protein beta 5 subunit mRNA, comp; 39969_at Cluster Incl. AA255502:zr85b06.r1 Homo sapiens cDNA, 5 end /clone=IMAG; 40343_at Cluster Incl. AJ005814:Homo sapiens mRNA for hoxA7 protein /cds=(106,79; 41005_at Cluster Incl. L36531:Homo sapiens integrin alpha 8 subunit mRNA, 3 end; 41091_at Cluster Incl. U05237:Human fetal Alz-50-reactive clone 1 (FAC1) mRNA, c; 41466_s_at Cluster Incl. L04282:Human CACCC box-binding protein mRNA, complete c; 32129_at Cluster Incl. AL079314:Homo sapiens mRNA full length insert cDNA clone; 32734_at Cluster Incl. L76703:Homo sapiens protein phosphatase 2A B56-epsilon (P; 35725_at Cluster Incl. D89618:Homo sapiens mRNA for karyopherin alhph 3, complet; 35985_at Cluster Incl. AB023137:Homo sapiens mRNA for KIAA0920 protein, complete; 38639_at Cluster Incl. AF040963:Homo sapiens Mad4 homolog (Mad4) mRNA, complete; 39419_at Cluster Incl. AB011088:Homo sapiens mRNA for KIAA0516 protein, partial; 40463_at Cluster Incl. U70322:Human transportin (TRN) mRNA, complete cds /cds=(9; 40464_g_at Cluster Incl. U70322:Human transportin (TRN) mRNA, complete cds /cds=; 40478_at Cluster Incl. AL021396:Human DNA sequence from clone 971N18 on chromoso; 32159_at Cluster Incl. L00049:Human cellular c-Ki-ras2 proto-oncogene, 5 flank a; 32815_at Cluster Incl. AI687419:tp95h03.x1 Homo sapiens cDNA, 3 end /clone=IMAG; 33381_at Cluster Incl. AF012108:Homo sapiens Amplified in Breast Cancer (AB1) m; 35295_g_at Cluster Incl. M25077:Human SS-A/Ro ribonucleoprotein autoantigen 60 k; 38420_at Cluster Incl. Y14690:Homo sapiens mRNA for procollagen alpha 2(V) /cds=; 40260_g_at Cluster Incl. AL009266:H. sapiens cDNA similar to C. elegans RNA bind; 40589_at Cluster Incl. U40572:Human beta2-syntrophin (SNT B2) mRNA, complete cds; 40990_at Cluster Incl. AF065389:Homo sapiens tetraspan NET-4 mRNA, complete cds; 41591_at Cluster Incl. AI652978:wb42a05.x1 Homo sapiens cDNA, 3 end /clone=IMAG; 1937_at Retinoblastoma 1; 1603_g_at L33881 /FEATURE= /DEFINITION=HUMPKCI Human protein kinase C iota isofo; 1537_at X00588 /FEATURE=cds /DEFINITION=HSEGFPRE Human mRNA for precursor of epi; 1258_s_at L76568 /FEATURE=exon#5 /DEFINITION=HUMERCC4G Homo sapiens excision and; 1124_at L04731 /FEATURE= /DEFINITION=HUMTRLALL1 Homo sapiens translocation T(4:1; 933_f_at L11672 /FEATURE= /DEFINITION=HUMKRUPZN Human Kruppel related zinc finge; 342_at D12485 /FEATURE= /DEFINITION=HUMNPP Human mRNA for nucleotide pyrophospha; 274_at L04282 /FEATURE= /DEFINITION=HUMTB Human CACCC box-binding protein mRNA,; 199_s_at U33052 /FEATURE= /DEFINITION=HSU33052 Human lipid-activated, protein ki.

Metagene 14; 34104_i_at Cluster Incl. AI147237:qb36f02.x1 Homo sapiens cDNA, 3 end /clone=IM; 37527_at Cluster Incl. Z36715:H. sapiens mRNA for Net transcription factor /cds=(; 31833_at Cluster Incl. U78575:Human 68 kDa type I phosphatidylinositol-4-phospha; 34747_at Cluster Incl. X83535:H. sapiens mRNA for membrane-type matrix metallopro; 36675_r_at Cluster Incl. J03191:Human profilin mRNA, complete cds /cds=(127,549); 36977_at Cluster Incl. U39412:Homo sapiens alpha SNAP mRNA, complete cds /cds=(6; 38757_at Cluster Incl. U41745:Human PDGF associated protein mRNA, complete cds /; 39182_at Cluster Incl. U87947:Human hematopoietic neural membrane protein (HNMP-; 39183_at Cluster Incl. X66363:H. sapiens mRNA PCTAIRE-1 for serine/threonine prot; 39835_at Cluster Incl. U93181:Homo sapiens nuclear dual-specificity phosphatase; 41551_at Cluster Incl. AW044624:wy78c04.x1 Homo sapiens cDNA, 3 end /clone=IMAG; 471_f_at U47634 /FEATURE= /DEFINITION=HSU47634 Human beta-tubulin class III isot; 429_f_at X00734 /FEATURE=cds /DEFINITION=HSREP10 Human beta-tubulin gene (5-beta.

Metagene 15; 31597_r_at Cluster Incl. L36055:Human 4E-binding protein 1 mRNA, complete cds /c; 36728_at Cluster Incl. M76446:Human alpha-A1-adrenergic receptor mRNA, complete; 36983_f_at Cluster Incl. X00442:Human mRNA for haptoglobin alpha(2FS)-beta precu; 204_at M74297 /FEATURE= /DEFINITION=HUMHOX14 Human homeobox 1.4 protein mRNA, co.

Metagene 16; 33660_at Cluster Incl. U14966:Human ribosomal protein L5 mRNA, complete cds /cds; 34604_at Cluster Incl. L05568:Human Na+/Cl− dependent serotonin transporter mRNA; 32345_at Cluster Incl. AL109696:Homo sapiens mRNA full length insert cDNA clone; 37159_at Cluster Incl. U79259:Human clone 23945 mRNA, complete cds /cds=(636,140; 37446_at Cluster Incl. AB007903:Homo sapiens KIAA0443 mRNA, complete cds /cds=(6; 37839_at Cluster Incl. AL109700:Homo sapiens mRNA full length insert cDNA clone; 39939_at Cluster Incl. D21337:Human mRNA for collagen /cds=(234,5270) /gb=D21337; 41013_at Cluster Incl. AL080114:Homo sapiens mRNA; cDNA DKFZp586M2022 (from clon; 41043_at Cluster Incl. AB006867:Homo sapiens mRNA for hSOX20 protein, complete c; 41478_at Cluster Incl. AL033538:Human DNA sequence from clone 477H23 on chromoso; 32674_at Cluster Incl. D83032:Homo sapiens mRNA for nuclear protein, NP220, comp; 33255_at Cluster Incl. M97856:Homo sapiens histone-binding protein mRNA, complet; 35142_at Cluster Incl. AF070617:Homo sapiens clone 24812 mRNA sequence /cds=UNKN; 36049_at Cluster Incl. W27899:39c4 Homo sapiens cDNA /gb=W27899 /gi=1307847 /ug=; 36890_at Cluster Incl. AF001691:Homo sapiens 195 kDa cornified envelope precurso; 38681_at Cluster Incl. U62962:Human Int-6 mRNA, complete cds /cds=(22,1359) /gb=; 38982_at Cluster Incl. W28865:53g9 Homo sapiens cDNA /gb=W28865 /gi=1308876 /ug=; 39765_at Cluster Incl. AB002318:Human mRNA for KIAA0320 gene, partial cds /cds=(; 40069_at Cluster Incl. AF051850:Homo sapiens supervillin mRNA, complete cds /cds; 40828_at Cluster Incl. D63476:Human mRNA for KIAA0142 gene, complete cds /cds=(4; 41142_at Cluster Incl. U62961:Human succinyl CoA-3-oxoacid CoA transferase precu; 33819_at Cluster Incl. X13794:H. sapiens lactate dehydrogenase B gene exon 1 and; 33910_at Cluster Incl. AL049338:Homo sapiens mRNA; cDNA DKFZp564P116 (from clone; 33911_at Cluster Incl. AI765053:wh56e11.x1 Homo sapiens cDNA, 3 end /clone=IMAG; 34323_at Cluster Incl. AF084260:Homo sapiens signalosome subunit 2 (SGN2) mRNA,; 35778_at Cluster Incl. AB011103:Homo sapiens mRNA for KIAA0531 protein, complete; 38816_at Cluster Incl. AF095791:Homo sapiens TACC2 protein (TACC2) mRNA, partial; 39556_at Cluster Incl. M96803:Human general beta-spectrin (SPTBN1) mRNA, complet; 41503_at Cluster Incl. AB020661:Homo sapiens mRNA for KIAA0854 protein, complete; 32521_at Cluster Incl. AF056087:Homo sapiens secreted frizzled related protein m; 1882_g_at Oncogene Aml1-Evi-1, Fusion Activated; 496_s_at U32324 /FEATURE= /DEFINITION=HSU32324 Human interleukin-11 receptor alp; 346_s_at D13814 /FEATURE= /DEFINITION=HUMAGRT1B Homo sapiens mRNA for angiotensi.

Metagene 17; 38981_at Cluster Incl. AA203354:zx58b07.r1 Homo sapiens cDNA, 5 end /clone=IMAG; 39020_at Cluster Incl. U82938:Human CD27BP (Siva) mRNA, complete cds /cds=(252,8; 33829_at Cluster Incl. Y12670:Homo sapiens mRNA for leptin receptor gene-related; 35331_at Cluster Incl. U97067:Homo sapiens alpha-catenin-like protein mRNA, comp; 35346_at Cluster Incl. AB007856:Homo sapiens KIAA0396 mRNA, partial cds /cds=(0,.

Metagene 18; 36367_at Cluster Incl. U79247:Human clone 23599 mRNA sequence /cds=UNKNOWN /gb=U; 33941_at Cluster Incl. AB009675:Homo sapiens mRNA for MDC/ADAM11, complete cds /; 34512_at Cluster Incl. J03853:Human kidney alpha-2-adrenergic receptor mRNA, com; 35457_at Cluster Incl. U20325:Human cocaine and amphetamine regulated transcript; 35673_at Cluster Incl. U02082:Human guanine nucleotide regulatory protein (tim1); 36459_at Cluster Incl. AB020686:Homo sapiens mRNA for KIAA0879 protein, complete; 39750_at Cluster Incl. W61005:zd29a11.s1 Homo sapiens cDNA, 3 end /clone=IMAGE-; 32853_at Cluster Incl. AB018262:Homo sapiens mRNA for KIAA0719 protein, complete; 33857_at Cluster Incl. N25122:yx19d10.r1 Homo sapiens cDNA, 5 end /clone=IMAGE-; 34384_at Cluster Incl. AF022853:untitled /cds=(0,4202) /gb=AF022853 /gi=2585771; 39878_at Cluster Incl. AI524125:th09d11.x1 Homo sapiens cDNA, 3 end /clone=IMAG; 40193_at Cluster Incl. X51956:Human ENO2 gene for neuron specific (gamma) enolas; 33137_at Cluster Incl. Y13622:Homo sapiens mRNA for latent transforming growth f.

Metagene 19; 39993_at Cluster Incl. D11466:Homo sapiens mRNA for PIG-A protein, complete cds; 39763_at Cluster Incl. M36803:Human hemopexin gene /cds=(28,1416) /gb=M36803 /gi; 34791_at Cluster Incl. X52882:Human t-complex polypeptide 1 gene /cds=(21,1691); 39860_at Cluster Incl. U05040:Human FUSE binding protein mRNA, complete cds /cds.

Metagene 20; 33587_f_at Cluster Incl. AI369146:qy74f11.x1 Homo sapiens cDNA, 3 end /clone=IM; 36423_at Cluster Incl. W47047:zc38g10.r1 Homo sapiens cDNA, 5 end /clone=IMAGE-; 38613_at Cluster Incl. U61837:Homo sapiens putative cyclin G1 interacting protei; 38754_at Cluster Incl. AI557295:PT2.1_{—}16_D02.r Homo sapiens cDNA, 3 end /clone_{—}; 40897_at Cluster Incl. M26061 Human cGMP phosphodiesterase alpha subunit (CGPR-A; 954_s_at Protein Phosphatase 1, Alpha Catalytic Subunit; 824_at U90313 /FEATURE= /DEFINITION=HSU90313 Human glutathione-S-transferase hom; 703_at Immunoglobulin Heavy Chain, Vdjrc Regions.

Metagene 21; 34538_at Cluster Incl. AL109682:Homo sapiens mRNA full length insert cDNA clone; 35441_at Cluster Incl. AF058922:Homo sapiens GLE1 (GLE1) mRNA, complete cds /cds; 37071_at Cluster Incl. AF042498:Homo sapiens rod photoreceptor CNG-channel beta; 38193_at Cluster Incl. X96754:H. sapiens gene encoding kappa light chain constant; 38942_r_at Cluster Incl. W28610:49b12 Homo sapiens cDNA /gb=W28610 /gi=1308558 /; 35978_at Cluster Incl. AF009242:Homo sapiens proline-rich G1a protein 1 (PRGP1); 33416_at Cluster Incl. AI332820:qp96e06.x1 Homo sapiens cDNA, 3 end /clone=IMAG; 34846_at Cluster Incl. AF112472:Homo sapiens calcium/calmodulin-dependent protei; 36178_at Cluster Incl. U23143:Human mitochondrial serine hydroxymethyltransferas; 37372_at Cluster Incl. M81780:Homo sapiens acid sphingomyelinase (SMPD1) gene, c; 38735_at Cluster Incl. AB011085:Homo sapiens mRNA for KIAA0513 protein, complete; 40173_at Cluster Incl. N32617:yw95f04.s1 Homo sapiens cDNA, 3 end /clone=IMAGE-; 1967_f_at U18334 /FEATURE=cds /DEFINITION=HSUNOSIIC1 Human nitric oxide synthase; 1292_at L11329 /FEATURE= /DEFINITION=HUMPAC1 Homo sapiens protein tyrosine phosp; 792_s_at X52611 /FEATURE=cds /DEFINITION=HSAP2 Human mRNA for transcription fact; 558_at M98776 /FEATURE=mRNA /DEFINITION=HUMKRT1X Human keratin 1 gene, complete.

Metagene 22; 31911_at Cluster Incl. AF000989:Homo sapiens thymosin beta 4 Y isoform (TB4Y) mR; 34586_s_at Cluster Incl. U51003:Human DLX-2 (Dlx2) mRNA, complete cds /cds=UNKNO; 35905_s_at Cluster Incl. U34995:Human normal keratinocyte substraction library m; 40336_at Cluster Incl. J03826:Human adrenodoxin reductase mRNA, complete cds /cd; 40342_at Cluster Incl. U66077:Human DAZ mRNA, 3UTR /cds=UNKNOWN /gb=U66077 /gi=1; 40376_at Cluster Incl. X83573:Homo sapiens ARSE gene, complete CDS /cds=(67,1836; 32080_at Cluster Incl. L11669:Human tetracycline transporter-like protein mRNA,; 35693_at Cluster Incl. AF070616:Homo sapiens clone 24772 BDP-1 protein mRNA, par; 36865_at Cluster Incl. AB018302:Homo sapiens mRNA for KIAA0759 protein, partial; 37912_at Cluster Incl. X80200:H. sapiens MLN62 mRNA /cds=(85,1497) /gb=X80200 /gi; 38290_at Cluster Incl. AF037195:Homo sapiens regulator of G protein signaling RG; 39801_at Cluster Incl. AF046889:Homo sapiens lysyl hydroxylase isoform 3 (PLOD3); 37047_at Cluster Incl. AF002020:Homo sapiens Niemann-Pick C disease protein (NPC; 38069_at Cluster Incl. Z67743:H. sapiens mRNA for CLC-7 chloride channel protein; 38813_at Cluster Incl. X75621:Homo sapiens TSC2 mRNA for tuberin /cds=(18,5441); 41586_at Cluster Incl. AF075292:Homo sapiens fibroblast growth factor 18 (FGF18); 1790_s_at Cell Division Cycle Protein 2-Related Protein Kinase (Piss1re); 720_at D87673 /FEATURE= /DEFINITION=D87673 Homo sapiens mRNA for heat shock tran; 505_at U43077 /FEATURE= /DEFINITION=HSU43077 Human CDC37 homolog mRNA, complete; 100_g_at Y08200 /FEATURE= /DEFINITION=HSRABGTRA Homo sapiens mRNA for rab gerany.

Metagene 23; 38174_at Cluster Incl. X99688:H. sapiens mRNA from TYL gene /cds=(1806,3743) /gb=; 38508_s_at Cluster Incl. U89337:Human HLA class III region containing cAMP respo; 31902_at Cluster Incl. AF093774:Homo sapiens type 2 iodothyronine deiodinase mRN; 40405_at Cluster Incl. X70991:H. sapiens MADER mRNA /cds=(0,1427) /gb=X70991 /gi=; 1337_s_at X06614 /FEATURE=cds /DEFINITION=HSRRA Human mRNA for receptor of retin; 684_at K02215 /FEATURE=mRNA#1 /DEFINITION=HUMANG Human angiotensinogen mRNA, com.

Metagene 24; 31472_s_at Cluster Incl. AF098641:Homo sapiens CD44 isoform RC (CD44) mRNA, comp; 37714_at Cluster Incl. M25667:Human neuronal growth protein 43 (GAP-43) mRNA, co; 2036_s_at M59040 /FEATURE= /DEFINITION=HUMCD44B Human cell adhesion molecule (CD; 1126_s_at L05424 /FEATURE=cds#5 /DEFINITION=HUMSCG19 Human cell surface glycopro.

Metagene 25; 31533_s_at Cluster Incl. AF048713:Homo sapiens Kv4.3 potassium channel long spli; 31739_at Cluster Incl. AA977513:on60e11.s1 Homo sapiens cDNA, 3 end /clone=IMAG; 39430_at Cluster Incl. AF082557:Homo sapiens TRF1-interacting ankyrin-related AD; 36200_at Cluster Incl. X69838:H. sapiens mRNA for G9a /cds=(47,3052) /gb=X69838 /; 36640_at Cluster Incl. X66141:H. sapiens mRNA for cardiac ventricular myosin ligh; 37001_at Cluster Incl. M23254:Human Ca2-activated neutral protease large subunit.

Metagene 26; 35442_at Cluster Incl. AB007958:Homo sapiens mRNA, chromosome 1 specific transcr; 40401_at Cluster Incl. AL050069:Homo sapiens mRNA; cDNA DKFZp566A0946 (from clon; 31885_at Cluster Incl. M64572:Human protein tyrosine phosphatase mRNA, complete; 36532_at Cluster Incl. AF039945:Homo sapiens synaptojanin 2B mRNA, partial cds /; 36873_at Cluster Incl. D16532:Human gene for very low density lipoprotein recept; 40454_at Cluster Incl. X87241:H. sapiens mRNA for hFat protein /cds=(186,13958) /; 32184_at Cluster Incl. X61118:Human TTG-2 mRNA for a cysteine rich protein with; 34795_at Cluster Incl. U84573:Homo sapiens lysyl hydroxylase isoform 2 (PLOD2) m; 36204_at Cluster Incl. Y00815:Human mRNA for LCA-homolog. LAR protein (leukocyte; 37350_at Cluster Incl. AL031177:dJ889N15.2.1 (26S Proteasome subunit p28 (Ankyri; 1458_at M64572 /FEATURE= /DEFINITION=HUMCAP Human protein tyrosine phosphatase m; 837_s_at U43944 /FEATURE= /DEFINITION=HSU43944 Human breast cancer cytosolic NAD.

Metagene 27; 35595_at Cluster Incl. AI557374:PT2.1_{—}6_C10.r Homo sapiens cDNA, 3 end /clone_e; 33985_s_at Cluster Incl. W28616:49b9 Homo sapiens cDNA /gb=W28616 /gi=1308564 /u; 33986_r_at Cluster Incl. W28616:49b9 Homo sapiens cDNA /gb=W28616 /gi=1308564 /u; 35468_at Cluster Incl. AL050381:Homo sapiens mRNA; cDNA DKFZp586B2023 (from clon; 37869_at Cluster Incl. AB029004:Homo sapiens mRNA for KIAA1081 protein, partial; 39962_at Cluster Incl. U59305:Human ser-thr protein kinase PK428 mRNA, complete; 34279_at Cluster Incl. AL050141:Homo sapiens mRNA; cDNA DKFZp586O031 (from clone; 38610_s_at Cluster Incl. X14487:Human gene for acidic (type I) cytokeratin 10 /c; 38701_at Cluster Incl. AJ000519:Homo sapiens mRNA for ubiquitin-conjugating enzy; 40137_at Cluster Incl. M31724:Human phosphotyrosyl-protein phosphatase (PTP-1B); 40150_at Cluster Incl. AA205857:zq50e04.r1 Homo sapiens cDNA, 5 end /clone=IMAG; 41233_at Cluster Incl. AB014888:Homo sapiens mRNA for MRJ, complete cds /cds=(10; 34891_at Cluster Incl. AI540958:PEC1.2_{—}15_H01.r Homo sapiens cDNA, 5 end /clone; 36209_at Cluster Incl. S78771:NAT=CpG island-associated gene [human, mRNA, 1741; 36611_at Cluster Incl. U25849:Human red cell-type low molecular weight acid phos; 36637_at Cluster Incl. L19605:Homo sapiens 56K autoantigen annexin XI gene mRNA,; 36982_at Cluster Incl. U30888:Human tRNA-guanine transglycosylase mRNA, complete; 39540_at Cluster Incl. AF000561:Homo sapiens TTF-I interacting peptide 21 mRNA,; 40535_i_at Cluster Incl. AI254524:qv48f07.x1 Homo sapiens cDNA, 3 end /clone=IM; 40536_f_at Cluster Incl. AI254524:qv48f07.x1 Homo sapiens cDNA, 3 end /clone=IM; 40959_at Cluster Incl. AB011171:Homo sapiens mRNA for KIAA0599 protein, partial; 41276_at Cluster Incl. W27641:37d11 Homo sapiens cDNA /gb=W27641 /gi=1307715 /ug; 41537_r_at Cluster Incl. L31881:Human nuclear factor I-X mRNA, complete cds /cds; 32509_at Cluster Incl. AI307607:tb15h10.x1 Homo sapiens cDNA, 3 end /clone=IMAG; 33172_at Cluster Incl. T75292:yc89b05.r1 Homo sapiens cDNA, 5 end /clone=IMAGE-; 1612_s_at X56681 /FEATURE=mRNA /DEFINITION=HSJUNDR Human junD mRNA; 1594_at J05448 /FEATURE= /DEFINITION=HUMRPOLAA Human RNA polymerase subunit hRPB; 1578_g_at M23263 /FEATURE= /DEFINITION=HUMARB Human androgen receptor mRNA, comp; 1460_g_at M68941 /FEATURE=mRNA /DEFINITION=HUMPTYPH Human protein-tyrosine phosp; 693_g_at Adenylyl Cyclase-Associated Protein 2; 588_at M31724 /FEATURE=mRNA /DEFINITION=HUMPTPBX Human phosphotyrosyl-protein ph; 237_s_at M60483 /FEATURE=mRNA /DEFINITION=HUMPP2AA Human protein phosphatase 2A.

Metagene 28; 31353_f_at Cluster Incl. X94553:H. sapiens HFKH4 mRNA for fork head like protein; 31399_at Cluster Incl. AF101441:Homo sapiens bone morphogenetic protein 10 (BMP1; 34093_at Cluster Incl. AI829701:wf09d11.x1 Homo sapiens cDNA, 3 end /clone=IMAG; 34154_at Cluster Incl. U62437:Human nicotinic acetylcholine receptor beta2 subun; 34172_s_at Cluster Incl. M99578:Human lymphocyte surface protein exons 1-5, comp; 32274_r_at Cluster Incl. AF052148:Homo sapiens clone 24507 mRNA sequence /cds=UN; 32874_at Cluster Incl. M65214:Human (HeLa) helix-loop-helix protein HE47 (E2A) m; 32907_at Cluster Incl. L41147:Homo sapiens 5-HT6 serotonin receptor mRNA, comple; 35911_r_at Cluster Incl. AJ003147:Homo sapiens complete genomic sequence between; 37095_r_at Cluster Incl. M84562:Human formyl peptide receptor-like receptor (FPR; 37414_at Cluster Incl. AF037066:Homo sapiens ORCTL2S hypothetical protein (ORCTL; 37442_at Cluster Incl. AL050378:Homo sapiens mRNA; cDNA DKFZp586I1420 (from clon; 39960_at Cluster Incl. AF091086:Homo sapiens clone 640 unknown mRNA, complete se; 40650_r_at Cluster Incl. X72304:H. sapiens mRNA for corticotrophin releasing fact; 41383_at Cluster Incl. AJ001403:Homo sapiens mNRA for MUC5AC protein (placental); 31804_f_at Cluster Incl. X78283:H. sapiens mRNA for aryl sulfotransferase (ST1A3); 34221_at Cluster Incl. D83778:Human mRNA for KIAA0194 gene, partial cds /cds=(0,; 36547_r_at Cluster Incl. AA521233:aa79a12.s1 Homo sapiens cDNA, 3 end /clone=IM; 37633_s_at Cluster Incl. J04129:Human placental protein 14 (PP14) mRNA, complete; 38356_at Cluster Incl. M19481:Human follistatin gene /cds=(0,953) /gb=M19481 /gi; 40499_r_at Cluster Incl. AF040708:Homo sapiens candidate tumor suppressor gene 2; 32756_at Cluster Incl. AF030249:Homo sapiens putative dienoyl-CoA isomerase (ECH; 33361_at Cluster Incl. AF052149:Homo sapiens clone 24733 mRNA sequence /cds=UNKN; 33826_at Cluster Incl. AL120500:DKFZp761M078_s1 Homo sapiens cDNA, 3 end /clone; 36173_r_at Cluster Incl. AF002163:Homo sapiens delta-adaptin mRNA, complete cds; 38056_at Cluster Incl. D83779:Human mRNA for KIAA0195 gene, complete cds /cds=(2; 38107_at Cluster Incl. U40998:Human retinal protein (HRG4) mRNA, complete cds /c; 38414_at Cluster Incl. U05340:Human p55CDC mRNA, complete cds /cds=(110,1609) /g; 38478_at Cluster Incl. U08377:Human homolog of Drosophila splicing regulator sup; 38741_at Cluster Incl. U70728:Human cytohesin-2 mRNA, complete cds /cds=(158,136; 39919_at Cluster Incl. AI423340:tf36c07.x1 Homo sapiens cDNA, 3 end /clone=IMAG; 40594_r_at Cluster Incl. Z22533:H. sapiens ALK-1 mRNA /cds=(282,1793) /gb=Z22533; 41255_at Cluster Incl. W28848:52g4 Homo sapiens cDNA /gb=W28848 /gi=1308814 /ug=; 41484_r_at Cluster Incl. X56681:Human junD mRNA /cds=(174,1217) /gb=X56681 /gi=3; 2091_at H23429 /FEATURE= /DEFINITION=H23429 ym52d12.s1 Soares infant brain 1NIB; 1998_i_at U19599 /FEATURE= /DEFINITION=HSU19599 Human (BAX delta) mRNA, complete; 1553_r_at U22028 /FEATURE=expanded-cds /DEFINITION=HSU22028 Human cytochrome P45; 1346_at S72043 /FEATURE=mRNA /DEFINITION=S72043 GIF=growth inhibitory factor [hu; 1277_at D89016 /FEATURE= /DEFINITION=D89016 Homo sapiens mRNA for Neuroblastoma,; 1131_at L11285 /FEATURE= /DEFINITION=HUMMEK2NF Homo sapiens ERK activator kinase; 854_at S76617 /FEATURE= /DEFINITION=S76617 blk=protein tyrosine kinase [human, B; 838_s_at U45328 /FEATURE= /DEFINITION=HSU45328 Human ubiquitin-conjugating enzym; 721_g_at D87673 /FEATURE= /DEFINITION=D87673 Homo sapiens mRNA for heat shock tr; 656_at L08488 /FEATURE= /DEFINITION=HUMINOS Human inositol polyphosphate 1-phosp; 240_at M64231 /FEATURE=mRNA /DEFINITION=HUMSPERSYN Human spermidine synthase gen.

Metagene 29; 31698_at Cluster Incl. Y08223:H. sapiens MFH-1 gene /cds=(0,1505) /gb=Y08223 /gi=; 32400_at Cluster Incl. D14539:Human mRNA for LTG19 /cds=(164,1843) /gb=D14539 /g; 34478_at Cluster Incl. X79780:H. sapiens YPT3 mRNA /cds=(6,662) /gb=X79780 /gi=76; 35426_at Cluster Incl. AC004410:Homo sapiens chromosome 19, fosmid 39554 /cds=(0; 35434_at Cluster Incl. L16794:Human transcription factor (MEF2) mRNA, complete c; 35438_at Cluster Incl. X87852:H. sapiens mRNA for SEX gene /cds=(184,5799) /gb=X8; 36733_at Cluster Incl. AI093511:qb08e02.x1 Homo sapiens cDNA, 3 end /clone=IMAG; 41420_at Cluster Incl. AF055033:Homo sapiens clone 24645 insulin-like growth fac; 33806_at Cluster Incl. AF052106:Homo sapiens clone 23781 mRNA sequence /cds=UNKN; 34722_at Cluster Incl. U44385:Human tissue inhibitor of metalloproteinases-2 (TI; 35155_at Cluster Incl. AC005306:Homo sapiens chromosome 19, cosmid R27216 /cds=(; 35242_at Cluster Incl. X66362:H. sapiens mRNA PCTAIRE-3 for serine/threonine prot; 38722_at Cluster Incl. X15880:Human mRNA for collagen VI alpha-1 C-terminal glob; 40873_at Cluster Incl. D86963:Human mRNA for KIAA0208 gene, complete cds /cds=(1; 32209_at Cluster Incl. AF052151:Homo sapiens clone 24574 mRNA sequence /cds=UNKN; 36180_s_at Cluster Incl. X75346:H. sapiens mRNA for MAP kinase activated protein; 40592_at Cluster Incl. L13329:Homo sapiens iduronate-2-sulfatase (IDS) gene /cds; 40972_at Cluster Incl. M77198:Human rac protein kinase beta mRNA, complete cds /; 32556_at Cluster Incl. X64044:H. sapiens mRNA for large subunit of splicing fact; 2076_s_at L37361 /FEATURE= /DEFINITION=HUMEFL3 Homo sapiens (clone hELK-L) ELK r; 1678_g_at M65062 /FEATURE= /DEFINITION=HUMIGFBP6 Human insulin-like growth facto; 1601_s_at L27559 /FEATURE=mRNA /DEFINITION=HUMIGFBP04 Human insulin-like growth; 1330_at U78876 /FEATURE= /DEFINITION=HSU78876 Human MEK kinase 3 mRNA, complete; 918_at Atp-Binding Cassette Protein; 845_at U16031 /FEATURE= /DEFINITION=HSU16031 Human transcription factor IL-4 Sta; 625_at L78833 /FEATURE=cds#4 /DEFINITION=HUMBRCA1 Human BRCA1, Rho7 and vatI gen; 612_s_at M19650 /FEATURE= /DEFINITION=HUMCNPDEA Human 2, 3-cyclic nucleotide 3; 569_g_at M80335 /FEATURE= /DEFINITION=HUMPKACATO Homo sapiens protein kinase A c; 242_at M64571 /FEATURE=mRNA /DEFINITION=HUMMAP4 Human microtubule-associated pro.

Metagene 30; 34459_at Cluster Incl. U50529:Human BRCA2 region, mRNA sequence CG016 /cds=UNKNO; 37794_at Cluster Incl. AF035281:Homo sapiens clone 23903 mRNA sequence /cds=UNKN; 34256_at Cluster Incl. AB018356:Homo sapiens mRNA for GM3 synthase, complete cds; 34743_at Cluster Incl. D63481:Human mRNA for KIAA0147 gene, partial cds /cds=(0,; 32800_at Cluster Incl. U66306:Human retinoid X receptor alpha mRNA, 3 UTR, parti; 39896_at Cluster Incl. AB011149:Homo sapiens mRNA for KIAA0577 protein, complete; 40195_at Cluster Incl. X14850:Human H2A.X mRNA encoding histone H2A.X /cds=(73,5; 1933_g_at U83661 /FEATURE= /DEFINITION=HSU83661 Homo sapiens multidrug resistanc.

Metagene 31; 34644_at Cluster Incl. AB021288:Homo sapiens mRNA for beta 2-microglobulin, comp; 37497_at Cluster Incl. L16499:Human orphan homeobox protein (PRH) mRNA, complete; 41405_at Cluster Incl. AF026692:Homo sapiens frizzled related protein frpHE mRNA; 32675_at Cluster Incl. D21878:Human mRNA for BST-1, complete cds /cds=(127,1083); 33236_at Cluster Incl. AF060228:Homo sapiens retinoic acid receptor responder 3; 36569_at Cluster Incl. X64559:H. sapiens mRNA for tetranectin /cds=(93,701) /gb=X; 36927_at Cluster Incl. AB0000115:Homo sapiens mRNA expressed in osteoblast, compl; 37641_at Cluster Incl. D28915:Human gene for hepatitis C-associated microtubular; 37975_at Cluster Incl. X04011:Human mRNA of X-CGD gene involved in chronic granu; 39409_at Cluster Incl. M14058:Human complement Clr mRNA, complete cds /cds=(63,2; 39424_at Cluster Incl. U70321:Human herpesvirus entry mediator mRNA, complete cd; 40153_at Cluster Incl. X57522:H. sapiens RING4 cDNA /cds=(30,2456) /gb=X57522 /gi; 40496_at Cluster Incl. J04080:Human complement component C1r mRNA, complete cds; 40766_at Cluster Incl. U24578:Human RP1 and complement C4B precursor (C4B) genes; 32814_at Cluster Incl. M24594:Human interferon-inducible 56 Kd protein mRNA, com; 33338_at Cluster Incl. M97936:Human transcription factor ISGF-3 mRNA sequence /c; 33453_at Cluster Incl. AI400326:tg89c03.x1 Homo sapiens cDNA, 3 end /clone=IMAG; 35311_at Cluster Incl. AF084523:Homo sapiens cellular repressor of E1A-stimulate; 35367_at Cluster Incl. AB006780:Homo sapiens mRNA for galectin-3, complete cds /; 35822_at Cluster Incl. L15702:Human complement factor B mRNA, complete cds /cds=; 36600_at Cluster Incl. L07633:Homo sapiens (clone 1950.2) interferon-gamma IEF S; 36672_at Cluster Incl. L13977:Human prolylcarboxypeptidase mRNA, complete cds /c; 37014_at Cluster Incl. M33882:Human p78 protein mRNA, complete cds /cds=(345,233; 38052_at Cluster Incl. M14539:Human factor XIII subunit a mRNA, 3 end /cds=(0,2; 915_at M24594 /FEATURE=mRNA /DEFINITION=HUMII56KD Human interferon-inducible 56.

Metagene 32; 33543_s_at Cluster Incl. U77718:Human desmosome associated protein pinin mRNA, c; 38510_at Cluster Incl. AL049435:Homo sapiens mRNA; cDNA DKFZp586B0220 (from clon; 39291_at Cluster Incl. X73874:H. sapiens PHKA 1 mRNA /cds=(161,3832) /gb=X73874 /; 40353_at Cluster Incl. AL049962:Homo sapiens mRNA; cDNA DKFZp564P0823 (from clon; 34191_at Cluster Incl. AB002445:Homo sapiens mRNA from chromosome 5q21-22, clone; 35199_at Cluster Incl. AB023199:Homo sapiens mRNA for KIAA0982 protein, complete; 35252_at Cluster Incl. AB011100:Homo sapiens mRNA for KIAA0528 protein, complete; 35695_at Cluster Incl. U67615:Human beige protein homolog (chs) mRNA, complete c; 35722_at Cluster Incl. AL080198:Homo sapiens mRNA; cDNA DKFZp434D222 (from clone; 37928_at Cluster Incl. AA621555:af53a04.s1 Homo sapiens cDNA, 3 end /clone=IMAG; 38001_at Cluster Incl. L04569:Homo sapiens (clone hHT-1) L-type voltage-dependen; 39035_at Cluster Incl. AF006010:Human progestin induced protein (DD5) mRNA, comp; 39354_at Cluster Incl. D14662:Human mRNA for KIAA0106 gene, complete cds /cds=(4; 39444_at Cluster Incl. AF054284:Homo sapiens spliceosomal protein SAP 155 mRNA,; 39699_at Cluster Incl. D28476:Human mRNA for KIAA0045 gene, complete cds /cds=(1; 40063_at Cluster Incl. U22897:Homo sapiens nuclear domain 10 protein (ndp52) mRN; 40102_at Cluster Incl. AB018315:Homo sapiens mRNA for KIAA0772 protein, complete; 40832_s_at Cluster Incl. AL050126:Homo sapiens mRNA; cDNA DKFZp586G011 (from clo; 40839_at Cluster Incl. AL080177:Homo sapiens mRNA; cDNA DKFZp434K151 (from clone; 40868_at Cluster Incl. AA442799:zv69b10.r1 Homo sapiens cDNA, 5 end /clone=IMAG; 41147_at Cluster Incl. AF038186:Homo sapiens clone 23914 mRNA sequence /cds=UNKN; 32798_at Cluster Incl. AF043105:Homo sapiens glutathione S-transferase mu 3 (GST; 32835_at Cluster Incl. AA725102:ai08h05.s1 Homo sapiens cDNA, 3 end /clone=1342; 33447_at Cluster Incl. X54304:Human mRNA for myosin regulatory light chain /cds=; 34394_at Cluster Incl. AB018327:Homo sapiens mRNA for KIAA0784 protein, partial; 34397_at Cluster Incl. AF069250:Homo sapiens okadaic acid-inducible phosphoprote; 34785_at Cluster Incl. AB028948:Homo sapiens mRNA for KIAA1025 protein, partial; 34786_at Cluster Incl. AB018285:Homo sapiens mRNA for KIAA0742 protein, partial; 34797_at Cluster Incl. AF014402:Homo sapiens type-2 phosphatidic acid phosphatas; 35317_at Cluster Incl. AB014579:Homo sapiens mRNA for KIAA0679 protein, partial; 35802_at Cluster Incl. AB023231:Homo sapiens mRNA for KIAA1014 protein, partial; 37007_at Cluster Incl. U49188:Human placenta (DifB33) mRNA, complete cds /cds=(1; 38070_at Cluster Incl. AL080234:Homo sapiens mRNA; cDNA DKFZp586L081 (from clone; 38405_at Cluster Incl. U25165:Human fragile X mental retardation protein 1 homol; 39897_at Cluster Incl. N36997:yy39g07.s1 Homo sapiens cDNA, 3 end /clone=IMAGE-; 41333_at Cluster Incl. D26069:Human mRNA for KIAA0041 gene, partial cds /cds=(0,; 41488_at Cluster Incl. AC002394:Human Chromosome 16 BAC clone CIT987SK-A-211C6 /; 32597_at Cluster Incl. X76061:H. sapiens p130 mRNA for 130K protein /cds=(69,3488; 1913_at U47414 /FEATURE= /DEFINITION=HSU47414 Human cyclin G2 mRNA, complete cds; 1420_s_at D30655 /FEATURE= /DEFINITION=HUMELF4AII Homo sapiens mRNA for eukaryot; 603_at M29960 /FEATURE=mRNA /DEFINITION=HUMTR211 Human steroid receptor (TR2-11); 176_at U37352 /FEATURE= /DEFINITION=HSU37352 Human protein phosphatase 2A B alph.

Metagene 33; 36403_s_at Cluster Incl. AI434146:ti36g07.x1 Homo sapiens cDNA, 3 end /clone=IM; 37411_at Cluster Incl. D30758:Human mRNA for KIAA0050 gene, complete cds /cds=(1; 38362_at Cluster Incl. W27545:32c4 Homo sapiens cDNA /gb=W27545 /gi=1307349 /ug=.

Metagene 34; 33085_at Cluster Incl. U64863:Human hPD-1 (hPD-1) mRNA, complete cds /cds=(68,93; 32919_at Cluster Incl. AC004010:Human BAC clone GS099H08 /cds=(0,1568) /gb=AC004; 36226_r_at Cluster Incl. W27611:35b9 Homo sapiens cDNA /gb=W27611 /gi=1307559 /u; 32741_at Cluster Incl. X77723:H. sapiens mRNA for unknown protein of uterine endo; 39466_s_at Cluster Incl. W27995:43e12 Homo sapiens cDNA /gb=W27995 /gi=1308150 /.

Metagene 35; 32284_at Cluster Incl. AF012130:Homo sapiens brachyury variant A (TBX1) mRNA, co; 41736_g_at Cluster Incl. AI808958:wf67a09.x1 Homo sapiens cDNA, 3 end /clone=IM; 37681_i_at Cluster Incl. AB018266:Homo sapiens mRNA for KIAA0723 protein, comple; 39849_at Cluster Incl. AF043473:Homo sapiens delayed-rectifier K+ channel alpha.

Metagene 36; 34149_at Cluster Incl. W28558:48f7 Homo sapiens cDNA /gb=W28558 /gi=1308524 /ug=; 34460_at Cluster Incl. AB014512:Homo sapiens mRNA for KIAA0612 protein, partial; 37772_at Cluster Incl. AB020711:Homo sapiens mRNA for KIAA0904 protein, partial; 41026_f_at Cluster Incl. U05255:Human glycophorin HeP2 mRNA, partial cds /cds=(0; 33220_at Cluster Incl. Z11773:Homo sapiens mRNA for SRE-ZBP /cds=(0,1226) /gb=Z1; 33221_at Cluster Incl. U80735:Homo sapiens CAGF28 mRNA, partial cds /cds=(0,2235; 36923_at Cluster Incl. M98045:Homo sapiens folylpolyglutamate synthetase mRNA, c; 32240_at Cluster Incl. D31889:Human mRNA for KIAA0072 gene, partial cds /cds=(0,; 640_at L48211 /FEATURE=cds /DEFINITION=HUMAIR Homo Sapiens angiotensin II recept.

Metagene 37; 41080_at Cluster Incl. AI218431:qh24d10.x1 Homo sapiens cDNA, 3 end /clone=IMAG; 31860_at Cluster Incl. X51804:Human PMI gene for a putative receptor protein /cd; 32137_at Cluster Incl. AF029778:Homo sapiens Jagged2 (JAG2) mRNA, complete cds /; 34833_at Cluster Incl. AL050157:Homo sapiens mRNA; cDNA DKFZp586O0120 (from clon; 41535_at Cluster Incl. AF006484:Homo sapiens putative oral tumor suppressor prot.

Metagene 38; 31600_s_at Cluster Incl. D38435:Homo sapiens hPMS3 mRNA, partial cds /cds=(0,772; 39664_at Cluster Incl. U28413:Human Cockayne syndrome complementation group A CS; 36088_at Cluster Incl. AJ006291:Homo sapiens mRNA for leucine rich protein /cds=; 37177_at Cluster Incl. Y00636:Human mRNA for lymphocyte function associated anti; 37610_at Cluster Incl. AI765280:wi73a08.x1 Homo sapiens cDNA, 3 end /clone=IMAG; 38275_at Cluster Incl. AF054996:Homo sapiens clone 23783 mRNA sequence /cds=UNKN; 39056_at Cluster Incl. X53793:H. sapiens ADE2H1 mRNA showing homologies to SAICAR; 39357_at Cluster Incl. U72514:Human C2f mRNA, complete cds /cds=(0,720) /gb=U725; 39416_at Cluster Incl. U90913:Human clone 23665 mRNA sequence /cds=UNKNOWN /gb=U; 40122_at Cluster Incl. AF037448:Homo sapiens RRM RNA binding protein Gry-rbp (GR; 40789_at Cluster Incl. U54645:Human adenylate kinase 2B (adk2b) gene, complete c; 41741_at Cluster Incl. U28686:Human putative RNA binding protein RNPL mRNA, comp; 32198_at Cluster Incl. W28979:54e8 Homo sapiens cDNA /gb=W28979 /gi=1308927 /ug=; 34798_at Cluster Incl. Z35491:H. sapiens mRNA for novel glucocorticoid receptor-a; 35353_at Cluster Incl. D11094:Human mRNA for MSS1, complete cds /cds=(66,1367) /; 36193_at Cluster Incl. U52522:Human arfaptin 2, putative target protein of ADP-r; 36652_at Cluster Incl. J03824:Human uroporphyrinogen III synthase mRNA, complete; 37044_at Cluster Incl. D49490:Homo sapiens mRNA for protein disulfide isomerase-; 37713_at Cluster Incl. L07548:Human aminoacylase-1 (ACY1) mRNA, complete cds /cd; 38811_at Cluster Incl. D82348:Homo sapiens mRNA for 5-aminoimidazole-4-carboxami; 32579_at Cluster Incl. U29175:Human transcriptional activator (BRG1) mRNA, compl; 32584_at Cluster Incl. D38047:Human mRNA for 26S proteasome subunit p31, complet; 32586_at Cluster Incl. D86971:Human mRNA for KIAA0217 gene, partial cds /cds=(0,; 1394_at L25080 /FEATURE= /DEFINITION=HUMRHOAA Homo sapiens GTP-binding protein (.

Metagene 39; 34011_at Cluster Incl. D83699:Human brain 3UTR of mRNA for neuronal death protei; 32691_s_at Cluster Incl. D86096:Human DNA for prostaglandin E receptor EP3 subty; 34704_r_at Cluster Incl. AA151971:zo30b03.r1 Homo sapiens cDNA, 5 end /clone=IM; 36139_at Cluster Incl. AL050289:Homo sapiens mRNA; cDNA DKFZp586G0522 (from clon; 37382_at Cluster Incl. N25117:yx19c09.r1 Homo sapiens cDNA, 5 end /clone=IMAGE-; 39870_at Cluster Incl. AI377866:te63h04.x1 Homo sapiens cDNA, 3 end /clone=IMAG; 40212_at Cluster Incl. AI123553:qa49h04.x1 Homo sapiens cDNA, 3 end /clone=IMAG; 41845_at Cluster Incl. W29036:55d12 Homo sapiens cDNA /gb=W29036 /gi=1308993 /ug; 1619_g_at D21241 /FEATURE=exon#1#3 /DEFINITION=HUMCP4B Human cytochrome P-450 ar; 1567_at S77812 /FEATURE= /DEFINITION=S77812 flt-vascular endothelial growth fact.

Metagene 40; 31337_at Cluster Incl. AF036329:Homo sapiens gonadotropin-releasing hormone prec; 35511_at Cluster Incl. AB014559:Homo sapiens mRNA for KIAA0659 protein, partial; 35484_at Cluster Incl. U95737:Human Chromosome 16 BAC clone CIT987SK-A-388D4 /cd; 36303_f_at Cluster Incl. U35376:Human repressor transcriptional factor (ZNF85) m; 35646_at Cluster Incl. Z35093:H. sapiens mRNA for SURF-1 /cds=(14,916) /gb=Z35093; 40416_at Cluster Incl. U04847:Human Ini1 mRNA, complete cds /cds=(69,1226) /gb=U; 40435_at Cluster Incl. J03592:Human ADP/ATP translocase mRNA, 3 end, clone pHAT; 41212_r_at Cluster Incl. D26068:Human mRNA for KIAA0038 gene, partial cds /cds=(; 32828_at Cluster Incl. AF026548:Homo sapiens branched chain alpha-ketoacid dehyd; 33889_s_at Cluster Incl. D79985:Human mRNA for KIAA0163 gene, complete cds /cds=; 35343_at Cluster Incl. M37400:Human cytosolic aspartate aminotransferase mRNA, c; 37723_at Cluster Incl. U47414:Human cyclin G2 mRNA, complete cds /cds=(135,1169); 38081_at Cluster Incl. J03459:Human leukotriene A-4 hydrolase mRNA, complete cds; 38381_at Cluster Incl. U32315:Human syntaxin 3 mRNA, complete cds /cds=(38,907); 39534_at Cluster Incl. L77564:Homo sapiens DGS-G mRNA, 3 end /cds=UNKNOWN /gb=L.

Metagene 41; 34931_at Cluster Incl. AB023157:Homo sapiens mRNA for KIAA0940 protein, complete; 37146_at Cluster Incl. AB007864:Homo sapiens KIAA0404 mRNA, partial cds /cds=(0,; 39637_at Cluster Incl. U14528:Human sulfate transporter (DTD) mRNA, complete cds; 32069_at Cluster Incl. AB014515:Homo sapiens mRNA for KIAA0615 protein, complete; 32721_at Cluster Incl. AA151922:zo30d07.r1 Homo sapiens cDNA, 5 end /clone=IMAG; 34199_at Cluster Incl. AJ131244:Homo sapiens mRNA for Sec24 protein (Sec24A isof; 34737_at Cluster Incl. AF058718:Homo sapiens putative 13 S Golgi transport compl; 35709_at Cluster Incl. AF038172:Homo sapiens clone 23923 mRNA sequence /cds=UNKN; 36857_at Cluster Incl. AF084513:Homo sapiens DNA repair exonuclease (REC1) mRNA,; 36926_at Cluster Incl. X80692:H. sapiens ERK3 mRNA /cds=(478,2643) /gb=X80692 /gi; 38271_at Cluster Incl. AB006626:Homo sapiens mRNA for KIAA0288 gene, complete cd; 38667_at Cluster Incl. AA189161:zq45g01.s1 Homo sapiens cDNA, 3 end /clone=IMAG; 40146_at Cluster Incl. AL080212:Homo sapiens mRNA; cDNA DKFZp586H0723 (from clon; 41174_at Cluster Incl. AF012086:Homo sapiens Ran binding protein 2 (RanBP2alpha); 32217_at Cluster Incl. AF052105:Homo sapiens clone 23965 mRNA sequence /cds=UNKN; 32222_at Cluster Incl. AA152202:zl06a03.s1 Homo sapiens cDNA, 3 end /clone=IMAG; 33342_at Cluster Incl. AF039029:Homo sapiens snurportin1 mRNA, complete cds /cds; 33830_at Cluster Incl. AW026535:wv14f10.x1 Homo sapiens cDNA, 3 end /clone=IMAG; 33896_at Cluster Incl. U01877:Human p300 protein mRNA, complete cds /cds=(1199,8; 34411_at Cluster Incl. Y10387:H. sapiens mRNA for PAPS synthetase /cds=(36,1910); 35303_at Cluster Incl. U96876:Homo sapiens insulin induced protein 1 (INSIG1) ge; 35838_at Cluster Incl. U90919:Human clones 23667 and 23775 zinc finger protein m; 35847_at Cluster Incl. AB028980:Homo sapiens mRNA for KIAA1057 protein, partial; 37306_at Cluster Incl. D38549:Human mRNA for KIAA0068 gene, partial cds /cds=(0,; 37735_at Cluster Incl. U31383:Human G protein gamma-10 subunit mRNA, complete cd; 37737_at Cluster Incl. D25547:Homo sapiens mRNA for PIMT isozyme I, complete cds; 38395_at Cluster Incl. X61100:Human mRNA for mitochondrial 75 kDa iron sulphur p; 39923_at Cluster Incl. AI935420:wo84c08.x1 Homo sapiens cDNA, 3 end /clone=IMAG; 40167_s_at Cluster Incl. AF038187:Homo sapiens clone 23714 mRNA sequence /cds=UN; 40612_at Cluster Incl. AB029040:Homo sapiens mRNA for KIAA1117 protein, partial; 41790_at Cluster Incl. AL031230:dJ73M23.2 (NAD+-dependent succinic semialdehyde; 2063_at L20046 /FEATURE= /DEFINITION=HUMERCC5A Human ERCC5 excision repair prote; 1070_at M76766 /FEATURE= /DEFINITION=HUMTFIIB Human transcription factor (TFIIB); 706_at Glucocorticoid Receptor, Beta; 631_g_at L39874 /FEATURE=expanded-cds /DEFINITION=HUMDODDA Homo sapiens deoxycyt.

Metagene 42; 32954_at Cluster Incl. U79263:Human clone 23760 mRNA, partial cds /cds=(0,1021); 31903_at Cluster Incl. AB014593:Homo sapiens mRNA for KIAA0693 protein, partial; 35140_at Cluster Incl. R59697:yh11b03.s1 Homo sapiens cDNA, 3 end /clone=IMAGE-; 36506_at Cluster Incl. AJ131693:Homo sapiens mRNA for AKAP450 protein /cds=(222,; 38676_at Cluster Incl. AA059408:zl96e07.r1 Homo sapiens cDNA, 5 end /clone=IMAG; 32150_at Cluster Incl. X82834:H. sapiens mRNA for golgin /cds=(207,6764) /gb=X828; 34387_at Cluster Incl. D86960:Human mRNA for KIAA0205 gene, complete cds /cds=(2; 36099_at Cluster Incl. M69040:Human SF2p33 mRNA, complete cds /cds=(124,870) /gb; 36975_at Cluster Incl. W26659:34d2 Homo sapiens cDNA /gb=W26659 /gi=1307502 /ug=; 37693_at Cluster Incl. L40393:Homo sapiens (clone S171) mRNA, complete cds /cds=; 38473_at Cluster Incl. M63180:Human threonyl-tRNA synthetase mRNA, complete cds; 32563_at Cluster Incl. U51478:Human sodium/potassium-transporting ATPase beta-3; 1480_at L12723 /FEATURE= /DEFINITION=HUMHSP70H Human heat shock protein 70 (hsp7; 1446_at D00760 /FEATURE= /DEFINITION=HUMPSC3 Human mRNA for proteasome subunit H; 449_at U66469 /FEATURE= /DEFINITION=HSU66469 Human cell growth regulator CGR19 m; 350_at D28118 /FEATURE= /DEFINITION=HUMDB1 Human mRNA for DB1, complete cds; 193_at U21858 /FEATURE= /DEFINITION=HSU21858 Human transcriptional activation fa.

Metagene 43; 33091_at Cluster Incl. AF005220:Homo sapiens transcription factor HOXD13 (Hoxd13; 35918_at Cluster Incl. AB020522:Homo sapiens DLEC1 (deleted in lung and esophage; 41064_at Cluster Incl. AL049296:Homo sapiens mRNA; cDNA DKFZp564P013 (from clone; 40047_at Cluster Incl. AF077599:Homo sapiens hypothetical SBBI03 protein mRNA, c; 35328_at Cluster Incl. AF055023:Homo sapiens clone 24723 mRNA sequence /cds=UNKN; 39543_at Cluster Incl. AI077476:oz49b01.x1 Homo sapiens cDNA, 3 end /clone=IMAG; 1464_at S73149 /FEATURE=mRNA /DEFINITION=S73149 insulin-like growth factor II {i; 917_g_at L18983 /FEATURE=mRNA /DEFINITION=HUMTYROPHO Homo sapiens tyrosine phosp; 477_at U51127 /FEATURE= /DEFINITION=HSU51127 Human interferon regulatory factor.

Metagene 44; 34649_at Cluster Incl. M14219:Human chondroitin/dermatan sulfate proteoglycan (P; 32324_at Cluster Incl. X57346:H. sapiens mRNA for HS1 protein /cds=(372,1112) /gb; 38233_at Cluster Incl. AF093265:Homo sapiens homer-3 mRNA, complete cds /cds=(90; 41662_at Cluster Incl. AL050272:Homo sapiens mRNA; cDNA DKFZp566B183 (from clone; 37611_at Cluster Incl. AB008822:Homo sapiens gene for osteoclastogenesis inhibit; 37948_at Cluster Incl. J05682:Human subunit C of V-ATPase (vat C) mRNA, 3 end /; 39733_at Cluster Incl. AF055001:Homo sapiens clone 24560 unknown mRNA, complete; 34818_at Cluster Incl. X96381:H. sapiens erm gene, exon 2, 3, 4, 5 (and joined CDS); 34857_at Cluster Incl. Z24724:H. sapiens polyA site DNA /cds=UNKNOWN /gb=Z24724 /; 35799_at Cluster Incl. AL080081:Homo sapiens mRNA; cDNA DKFZp564F1862 (from clon; 38111_at Cluster Incl. X15998:H. sapiens mRNA for the chondroitin sulphate proteo; 38112_g_at Cluster Incl. X15998:H. sapiens mRNA for the chondroitin sulphate prot; 41352_at Cluster Incl. X62822:H. sapiens gene encoding beta-galactoside alpha-2,6.

Metagene 45; 33623_g_at Cluster Incl. Z26308:H. sapiens isoform 1 gene for L-type calcium chan; 34139_at Cluster Incl. AL049651:Human DNA sequence from clone 753D10 on chromoso; 34146_at Cluster Incl. AB019529:Homo sapiens mRNA for OGG1 protein type 2c, part; 34241_at Cluster Incl. L34357:Homo sapiens GATA-4 mRNA, complete cds /cds=(240,1; 39554_at Cluster Incl. AI186701:qe82d12.x1 Homo sapiens cDNA, 3 end /clone=IMAG.

Metagene 46; 38148_at Cluster Incl. D83702:Homo sapiens mRNA for photolyase, complete cds /cd; 39219_at Cluster Incl. U20240:Human C/EBP gamma mRNA, complete cds /cds=(250,702; 41027_at Cluster Incl. AF078096:Homo sapiens forkhead/winged helix-like transcri; 31897_at Cluster Incl. U53445:Human ovarian cancer downregulated myosin heavy ch; 33730_at Cluster Incl. AF095448:Homo sapiens putative G protein-coupled receptor; 36536_at Cluster Incl. AF070614:Homo sapiens clone 24732 unknown mRNA, partial c; 38704_at Cluster Incl. AB007934:Homo sapiens mRNA for KIAA0465 protein, partial; 39329_at Cluster Incl. X15804:Human mRNA for alpha-actinin /cds=(198,2876) /gb=X; 39748_at Cluster Incl. AL050021:Homo sapiens mRNA; cDNA DKFZp564D016 (from clone; 40785_g_at Cluster Incl. Z69030:H. sapiens mRNA for gamma 1 isoform of 61 kDa regu; 41137_at Cluster Incl. AB007972:Homo sapiens mRNA, chromosome 1 specific transcr; 34800_at Cluster Incl. AL039458:DKFZp434N0910_s1 Homo sapiens cDNA, 3 end /clon; 36143_at Cluster Incl. U13737:Human cysteine protease CPP32 isoform alpha mRNA,; 37679_at Cluster Incl. Y10313:Homo sapiens mRNA IFRD1 (PC4) interferon-related d; 41523_at Cluster Incl. U59878:Human low-Mr GTP-binding protein (RAB32) mRNA, par; 1638_at U11732 /FEATURE= /DEFINITION=HSU11732 Human ets-like gene (tel) mRNA, co; 1529_at U50534 /FEATURE= /DEFINITION=HSU50534 Human BRCA2 region, mRNA sequence; 670_s_at L05515 /FEATURE= /DEFINITION=HUMCREBPA Homo sapiens cAMP response eleme; 232_at M55210 /FEATURE=mRNA#1 /DEFINITION=HUMLB2A26 Human laminin B2 chain gene,.

Metagene 47; 34474_at Cluster Incl. W28429:49e1 Homo sapiens cDNA /gb=W28429 /gi=1308584 /ug=; 32107_at Cluster Incl. AL050173:Homo sapiens mRNA; cDNA DKFZp586F0422 (from clon; 33275_at Cluster Incl. AB016194:Homo sapiens elk1 oncogene, complete cds /cds=(3; 35992_at Cluster Incl. AF087036:Homo sapiens musculin mRNA, partial cds /cds=(0,; 38618_at Cluster Incl. AC002073:Human PAC clone DJ515N1 from 22q11.2-q22 /cds=(0; 38651_at Cluster Incl. U60061:HumanFEZ2 mRNA, partial cds /cds=(0,461) /gb=U600; 40422_at Cluster Incl. X16302:Human mRNA for insulin-like growth factor binding; 33925_at Cluster Incl. X99076:H. sapiens NRGN gene, exons 2, 3 & 4 (oined CDS) /c; 35828_at Cluster Incl. D42123:Homo sapiens mRNA for ESP1 /CRP2, complete cds /cds; 36950_at Cluster Incl. X90872:H. sapiens mRNA for gp25L2 protein /cds=(91,735) /g; 40560_at Cluster Incl. U28049:Human TBX2 (TXB2) mRNA, complete cds /cds=(47,2155; 1741_s_at S37730 /FEATURE=cds /DEFINITION=S37712S4 insulin-like growth factor bi; 996_at X59065 /FEATURE=exon /DEFINITION=HSFGFEX3 H. sapiens FGF gene, exon 3; 941_at D29012 /FEATURE= /DEFINITION=HUMPSY Human mRNA for proteasome subunit Y,; 885_g_at M59911 /FEATURE= /DEFINITION=HUMINTA3A Human integrin alpha-3 chain mRN.

Metagene 48; 34128_at Cluster Incl. AB020337:Homo sapiens mRNA for UDP-Gal-GlcNAc beta1,3-gal; 34197_at Cluster Incl. X80907:H. sapiens mRNA for p85 beta subunit of phosphatidy; 35179_at Cluster Incl. AB009598:Homo sapiens mRNA for glucuronyltransferase I, c; 36897_at Cluster Incl. D25217:Human mRNA for KIAA0027 gene, partial cds /cds=(0,; 40521_at Cluster Incl. AL050259:Homo sapiens mRNA; cDNA DKFZp547D0710 (from clon; 39542_at Cluster Incl. AF059611:Homo sapiens nuclear matrix protein NRP/B (NRPB); 40614_at Cluster Incl. X75342:H. sapiens SHB mRNA /cds=(310,2100) /gb=X75342 /gi=; 1726_at Dna Polymerase, Epsilon, Catalytic Subunit; 1724_at S75174 /FEATURE= /DEFINITION=S75174 E2F-4=transcription factor [human, N; 1271_gnat L19067 /FEATURE= /DEFINITION=HUMNFKB65A Human NF-kappa-B transcription; 1202_g_at D14889 /FEATURE= /DEFINITION=HUMSGBP Human mRNA for small GTP-binding.

Metagene 49; 1803_at X05360 /FEATURE=cds /DEFINITION=HSCDC2 Human CDC2 gene involved in cell; 434_at X03473 /FEATURE=cds /DEFINITION=HSHIS10G Human gene for histone H1(0).

Metagene 50; 31389_at Cluster Incl. U46752:Human phosphotyrosine independent ligand p62B B-ce; 33033_at Cluster Incl. W26326:29a10 Homo sapiens cDNA /gb=W26326 /gi=1307088 /ug; 34589_f_at Cluster Incl. AC002366:Human Xp22 BAC CT-28515 (from CalTech/Researc; 36275_at Cluster Incl. AB002438:Homo sapiens mRNA from chromosome 5q21-22, clone; 38541_at Cluster Incl. M12792:Human steroid 21-hydroxylase [P-450(C21)] A pseudo; 31793_at Cluster Incl. AL036554:DKFZp564J2262_r1 Homo sapiens cDNA, 5 end /clon; 37204_at Cluster Incl. X67055:H. sapiens mRNA for inter-alpha-trypsin inhibitor h; 37950_at Cluster Incl. X74496:H. sapiens mRNA for prolyl oligopeptidase /cds=(0,2; 37972_at Cluster Incl. U75744:Homo sapiens DNase gamma mRNA, complete cds /cds=(; 34333_at Cluster Incl. AL021707:Human DNA sequence from clone 508115 on chromoso; 2000_at U26455 /FEATURE= /DEFINITION=HSU26455 Human phosphatidylinositol 3-kinas; 1370_at M29696 /FEATURE= /DEFINITION=HUMIL7AA Human interleukin-7 receptor (IL-7; 1325_at U59423 /FEATURE= /DEFINITION=HSU59423 Human Smad1 mRNA, complete cds; 733_at Mucin.

Metagene 51; 31492_at Cluster Incl. AB019392:Homo sapiens mRNA of muscle specific gene M9, co; 32454_at Cluster Incl. AL080215:Homo sapiens mRNA; cDNA DKFZp586J0323 (from clon; 38211_at Cluster Incl. AL050276:Homo sapiens mRNA; cDNA DKFZp566F123 (from clone; 39682_at Cluster Incl. X87159:H. sapiens mRNA for beta subunit of epithelial ami1; 41606_at Cluster Incl. AJ005940:Homo sapiens mRNA for GTP-binding protein /cds=(; 41644_at Cluster Incl. AB018333:Homo sapiens mRNA for KIAA0790 protein, partial; 32713_at Cluster Incl. U51587:Homo sapiens Golgi complex autoantigen golgin-97 m; 33795_at Cluster Incl. AB006630:Homo sapiens mRNA for KIAA0292 gene, partial cds; 34703_f_at Cluster Incl. AA151971:zo30b03.r1 Homo sapiens cDNA, 5 end /clone=IM; 39412_at Cluster Incl. U09825:Human acid finger protein mRNA, complete cds /cds=; 40428_i_at Cluster Incl. AW043812:wy81b07.x1 Homo sapiens cDNA, 3 end /clone=IM; 40765_at Cluster Incl. D87438:Human mRNA for KIAA0251 gene, partial cds /cds=(0,; 41161_at Cluster Incl. AB015051:Homo sapiens mRNA for Daxx, complete cds /cds=(1; 32261_at Cluster Incl. AF072810:Homo sapiens transcription factor WSTF mRNA, com; 33371_s_at Cluster Incl. U59877:Human low-Mr GTP-binding protein (RAB31) mRNA, c; 34839_at Cluster Incl. AB029027:Homo sapiens mRNA for KIAA1104 protein, complete; 34841_at Cluster Incl. AC002544:Homo sapiens Chromosome 16 BAC clone CIT987SK-A-; 34849_at Cluster Incl. X91257:H. sapiens mRNA for seryl-tRNA synthetase /cds=(75,; 36186_at Cluster Incl. L37368:Human (clone E5.1) RNA-binding protein mRNA, compl; 32588_s_at Cluster Incl. X78992:H. sapiens ERF-2 mRNA /cds=(66,1544) /gb=X78992 /.

Metagene 52; 41451_s_at Cluster Incl. W28498:50e2 Homo sapiens cDNA /gb=W28498 /gi=1308653 /u; 41640_at Cluster Incl. AL031427:dJ167A19.1 (novel protein) /cds=(122,1042) /gb=A; 38000_at Cluster Incl. S72370:pyruvate carboxylase [human, kidney, mRNA, 4017 nt; 38292_at Cluster Incl. AF093264:Homo sapiens homer-2b mRNA, complete cds /cds=(0; 39752_at Cluster Incl. AF040704:Homo sapiens putative tumor suppressor protein (; 40124_at Cluster Incl. Y18418:Homo sapiens mRNA for erythrocyte cytosolic protei; 32151_at Cluster Incl. X82260:H. sapiens mRNA for RanGTPase activating protein 1; 41850_s_at Cluster Incl. U63825:Human hepatitis delta antigen interacting protei.

Metagene 53; 31684_at Cluster Incl. M62896:Human lipocortin (LIP) 2 pseudogene mRNA, complete; 36267_at Cluster Incl. X77909:H. sapiens IKBL mRNA /cds=(68,1213) /gb=X77909 /gi=; 40328_at Cluster Incl. X99268:H. sapiens mRNA for B-HLH DNA binding protein /cds=; 41699_f_at Cluster Incl. AL080149:Homo sapiens mRNA; cDNA DKFZp434B094 (from clo; 33292_at Cluster Incl. AL008583:dJ327J16.1 (human ortholog of mouse outer arm Dy; 36507_at Cluster Incl. D30612:Homo sapiens mRNA for repressor protein, partial c; 38251_at Cluster Incl. AI127424:qb75b02.x1 Homo sapiens cDNA, 3 end /clone=IMAG; 39369_at Cluster Incl. AB023152:Homo sapiens mRNA for KIAA0935 protein, partial; 32202_at Cluster Incl. U67322:Human HBV associated factor (XAP4) mRNA, complete; 37700_at Cluster Incl. X92106:H. sapiens mRNA for bleomycin hydrolase /cds=(78,14; 38812_at Cluster Incl. X79683:H. sapiens LAMB2 mRNA for beta2 laminin /cds=(165,5; 39133_at Cluster Incl. AI525379:PT1.1_{—}06_H01.r Homo sapiens cDNA, 5 end /clone_{—}; 39846_at Cluster Incl. AF071748:Homo sapiens cathepsin F (CATSF) mRNA, complete; 39861_at Cluster Incl. M98343:Homo sapiens amplaxin (EMS1) mRNA, complete cds /c; 40253_at Cluster Incl. AJ011123:Homo sapiens mRNA for phosphatidylinositol 4-kin; 41251_at Cluster Incl. L40410:Homo sapiens thyroid receptor interactor (TRIP3) m; 41530_at Cluster Incl. D16294:Human mRNA for mitochondrial 3-oxoacyl-CoA thiolas; 32527_at Cluster Incl. AI381790:te41h10.x1 Homo sapiens cDNA, 3 end /clone=IMAG; 1746_s_at Tumor Necrosis Factor Receptor 2 Associated Protein Trap3; 1257_s_at L42379 /FEATURE=mRNA /DEFINITION=HUMBPGF Homo sapiens bone-derived gro; 484_at U59302 /FEATURE= /DEFINITION=HSU59302 Human steroid receptor coactivator-; 197_at U29656 /FEATURE= /DEFINITION=HSU29656 Human DR-nm23 mRNA, complete cds; 198_g_at U29656 /FEATURE= /DEFINITION=HSU29656 Human DR-nm23 mRNA, complete cds.

Metagene 54; 31548_at Cluster Incl. L13291:Human ADP-ribosylarginine hydrolase mRNA, complete; 31690_at Cluster Incl. U08997:Human glutamate dehydrogenase gene, complete cds /; 31927_s_at Cluster Incl. D86062:Human mRNA for KNP-Ib, complete cds /cds=(18,731; 34170_s_at Cluster Incl. U56813:Human polycystwin mRNA, partial cds /cds=(0,1826; 36414_s_at Cluster Incl. AF032119:Homo sapiens hCASK (CASK) mRNA, complete cds /; 35886_at Cluster Incl. AL049758:dJ437M21.3 (novel Src homology domain 3 containi; 37528_at Cluster Incl. U03109:Human aspartyl beta-hydroxylase mRNA, complete cds; 37886_at Cluster Incl. AB015332:Homo sapiens HRIHFB2018 mRNA, partial cds /cds=(; 40737_at Cluster Incl. U13913:Human large-conductance calcium-activated potassiu; 41650_at Cluster Incl. U63810:Homo sapiens WD40 protein Ciao 1 mRNA, complete cd; 34680_s_at Cluster Incl. D14663:Human mRNA for KIAA0107 gene, complete cds /cds=; 36470_s_at Cluster Incl. U84551:Human dystrobrevin (DTN) gene /cds=(1,2232) /gb=; 39364_s_at Cluster Incl. Y18207:Homo sapiens mRNA for protein phosphatase 1 (PPP; 40101_g_at Cluster Incl. U72206:Human guanine nucleotide regulatory factor (LFP4; 32146_s_at Cluster Incl. L07261:Human alpha adducin mRNA, partial cds including; 33393_at Cluster Incl. AJ237946:Homo sapiens mRNA for DEAD Box Protein 5 /cds=(0; 40524_at Cluster Incl. X79510:H. sapiens mRNA for protein-tyrosine-phosphatase D1; 40545_at Cluster Incl. AB018566:Homo sapiens gene for Proline synthetase associa; 2037_s_at M60725 /FEATURE= /DEFINITION=HUMP70S6KB Human p70 ribosomal S6 kinase; 2018_at M65188 /FEATURE= /DEFINITION=HUMCX43 Human connexin 43 (GJA1, Cx43) mRNA; 1981_s_at X60287 /FEATURE=cds /DEFINITION=HSMAXM H. sapiens max mRNA; 1986_at X74594 /FEATURE=cds /DEFINITION=HSRB2P130 H. sapiens mRNA for Rb2 /p130 pr; 1912_s_at M74088 /FEATURE= /DEFINITION=HUMFAPAPC Human APC gene mRNA, complete c; 1837_at Ras-Like Protein Tc21; 1838_g_at Ras-Like Protein Tc21; 1581_s_at M27504 /FEATURE= /DEFINITION=HUMTOPIIX Homo sapiens topoisomerase type; 1382_at M63488 /FEATURE= /DEFINITION=HUMRPA70KD Human replication protein A 70 kD; 1320_at X79510 /FEATURE=cds /DEFINITION=HSPTPD1 H. sapiens mRNA for protein-tyros; 969_s_at X98296 /FEATURE=cds /DEFINITION=HSUBIQHYD H. sapiens mRNA for ubiquitin; 944_s_at D49354 /FEATURE= /DEFINITION=HUMHSP70A Human mRNA for enhancer protein; 704_at Nuclear Factor 1, A Type; 476_s_at U50079 /FEATURE= /DEFINITION=HSU50079 Human histone deacetylase HD1 mRN; 455_at U66618 /FEATURE= /DEFINITION=HSU66618 Human SWI/SNF complex 60 KDa subuni; 305_g_at Guanine Nucleotide Exchange Factor 2; 226_at M33336 /FEATURE= /DEFINITION=HUMCAMPPK Human cAMP-dependent protein kinas.

Metagene 55; 38152_at Cluster Incl. AI632589:wb10h03.x1 Homo sapiens cDNA, 3 end /clone=IMAG; 39612_at Cluster Incl. AL050061:Homo sapiens mRNA; cDNA DKFZp566J123 (from clone; 39621_at Cluster Incl. AB007928:Homo sapiens mRNA for KIAA0459 protein, partial; 33698_at Cluster Incl. AB028975:Homo sapiens mRNA for KIAA1052 protein, complete; 35651_at Cluster Incl. AF002715:Homo sapiens MAP kinase kinase kinase (MTK1) mRN; 35683_at Cluster Incl. AB020659:Homo sapiens mRNA for KIAA0852 protein, complete; 35994_at Cluster Incl. AC002398:Human DNA from chromosome 19-specific cosmid F25; 38273_at Cluster Incl. AJ006268:Homo sapiens mRNA for putative ATPase, partial /; 38679_g_at Cluster Incl. AA733050:zg79b05.s1 Homo sapiens cDNA, 3 end /clone=39; 39381_at Cluster Incl. AF055030:Homo sapiens clone 24538 mRNA sequence /cds=UNKN; 40846_g_at Cluster Incl. U10324:Human nuclear factor NF90 mRNA, complete cds /cd; 41146_at Cluster Incl. J03473:Human poly(ADP-ribose) synthetase mRNA, complete c; 37750_at Cluster Incl. AF049140:Homo sapiens MMS2 (MMS2) mRNA, complete cds /cds; 38384_at Cluster Incl. X54199:Human mRNA for GARS-AIRS-GART /cds=UNKNOWN /gb=X54; 38728_at Cluster Incl. D86978:Human mRNA for KIAA0225 gene, partial cds /cds=(0,; 38732_at Cluster Incl. X91788:H. sapiens mRNA for Ic1n protein /cds=(88,801) /gb=; 1698_g_at U71087 /FEATURE= /DEFINITION=HSU71087 Human MAP kinase kinase MEK5b mR; 1287_at J03473 /FEATURE=mRNA /DEFINITION=HUMRISDAD Human poly(ADP-ribose) synthe.

Metagene 56; 31375_at Cluster Incl. W28546:48c8 Homo sapiens cDNA /gb=W28546 /gi=1308494 /ug=; 31701_r_at Cluster Incl. X83412:H. sapiens B1 mRNA for mucin /cds=(0,515) /gb=X83; 31977_at Cluster Incl. M92432:Homo sapiens retinal guanylyl cyclase (retGC) mRNA; 34545_at Cluster Incl. AF070587:Homo sapiens clone 24741 mRNA sequence /cds=UNKN; 36375_at Cluster Incl. X74614:H. sapiens ODF2 (allele 2) gene for outer dense fib; 33539_at Cluster Incl. W28567:51e2 Homo sapiens cDNA /gb=W28567 /gi=1308722 /ug=; 36230_at Cluster Incl. AI624038:ts25h10.x1 Homo sapiens cDNA, 3 end /clone=IMAG; 40298_at Cluster Incl. AB014603:Homo sapiens mRNA for KIAA0703 protein, complete; 40714_at Cluster Incl. S82198:caldecrin=serum calcium-decreasing factor [human,; 41095_at Cluster Incl. X52221:H. sapiens ERCC2 gene, exons 1 & 2 (partial) /cds=U; 32029_at Cluster Incl. AF017995:Homo sapiens 3-phosphoinositide dependent protei; 32677_at Cluster Incl. AF000979:Homo sapiens testis-specific Basic Protein Y 1 (; 34291_at Cluster Incl. U07424:Human putative tRNA synthetase-like protein mRNA,; 36051_s_at Cluster Incl. X58199:Human mRNA for beta adducin /cds=(322,2502) /gb=; 36829_at Cluster Incl. AF022991:Homo sapiens Rigui (RIGUI) mRNA, complete cds /c; 37898_r_at Cluster Incl. AI985964:wr79d08.x1 Homo sapiens cDNA, 3 end /clone=IM; 37905_r_at Cluster Incl. X66436:H. sapiens hsr1 mRNA (partial) /cds=UNKNOWN /gb=X; 37925_r_at Cluster Incl. AJ245434:Homo sapiens mRNA for G3a protein (G3a gene, 1; 39766_r_at Cluster Incl. AI744294:tr08h04.x1 Homo sapiens cDNA, 3 end /clone=IM; 40160_at Cluster Incl. AL080109:Homo sapiens mRNA; cDNA DKFZp586G1822 (from clon; 32220_at Cluster Incl. D63874:Human mRNA for HMG-1, complete cds /cds=(76,723) /; 33828_at Cluster Incl. AF035262:Homo sapiens BAF57 (BAF57) gene, complete cds /c; 34837_at Cluster Incl. AB002374:Human mRNA for KIAA0376 gene, partial cds /cds=(; 35852_at Cluster Incl. AB014558:Homo sapiens mRNA for KIAA0658 protein, partial; 37379_at Cluster Incl. X81789:H. sapiens mRNA for splicing factor SF3a60 /cds=(56; 39167_r_at Cluster Incl. D83174:Human mRNA for collagen binding protein 2, compl; 39508_at Cluster Incl. AI201607:qb81b03.x1 Homo sapiens cDNA, 3 end /clone=IMAG; 40265_s_at Cluster Incl. AI401287:tg92b04.x1 Homo sapiens cDNA, 3 end /clone=IM; 41258_at Cluster Incl. N29665:yw73e06.s1 Homo sapiens cDNA, 3 end /clone=IMAGE-; 41532_at Cluster Incl. Y09723:H. sapiens mRNA for Miz-1 protein /cds=(126,2537) /; 41561_s_at Cluster Incl. AI651368:wb05d07.x1 Homo sapiens cDNA, 3 end /clone=IM; 41825_at Cluster Incl. W26652:34c6 Homo sapiens cDNA /gb=W26652 /gi=1307495 /ug=; 32540_at Cluster Incl. AI762547:wh92e05.x1 Homo sapiens cDNA, 3 end /clone=IMAG; 1524_at U46194 /FEATURE= /DEFINITION=HSU46194 Human renal cell carcinoma antigen; 1047_s_at U37055 /FEATURE=mRNA /DEFINITION=HSU37055 Human hepatocyte growth fact; 358_at AF000545 /FEATURE=cds /DEFINITION=HSAF000545 Homo sapiens putative purine; 315_at D45132 /FEATURE= /DEFINITION=HUMHOXY1 Homo sapiens mRNA for zinc-finger D.

Metagene 57; 32016_at Cluster Incl. AL050256:Novel human gene mapping to chomosome 22 /cds=(1; 35054_at Cluster Incl. AF035278:Homo sapiens clone 23676 mRNA sequence /cds=UNKN; 33471_g_at Cluster Incl. AF052177:Homo sapiens clone 24510 mRNA sequence /cds=UN; 38880_at Cluster Incl. AB011168:Homo sapiens mRNA for KIAA0596 protein, partial; 39232_at Cluster Incl. U09284:Human PINCH protein mRNA, complete cds /cds=(119,1; 40290_f_at Cluster Incl. L13972:Homo sapiens beta-galactoside alpha-2,3-sialyltr; 40291_r_at Cluster Incl. L13972:Homo sapiens beta-galactoside alpha-2,3-sialyltr; 41113_at Cluster Incl. AI871396:wl81f07.x1 Homo sapiens cDNA, 3 end /clone=IMAG; 31858_at Cluster Incl. X07315:Human gene for PP15 (placental protein 15) /cds=(9; 33303_at Cluster Incl. N21470:yx57e11.s1 Homo sapiens cDNA, 3 end /clone=IMAGE-; 33329_at Cluster Incl. X92857:H. sapiens mRNA for NFI/CAAT-binding transcription; 35160_at Cluster Incl. AF064491:Homo sapiens LIM homeobox protein cofactor (CLIM; 36841_at Cluster Incl. AA811338:ob81g05.s1 Homo sapiens cDNA /clone=IMAGE-133781; 40149_at Cluster Incl. AL049924:Homo sapiens mRNA; cDNA DKFZp547G1110 (from clon; 32811_at Cluster Incl. X98507:H. sapiens mRNA for myosin-I beta /cds=(65,3151) /g; 35812_at Cluster Incl. AJ133769:Homo sapiens mRNA for nuclear transport receptor; 36210_g_at Cluster Incl. S78771:NAT=CpG island-associated gene [human, mRNA, 174; 39202_at Cluster Incl. W26403:29b4 Homo sapiens cDNA /gb=W26403 /gi=1307102 /ug=; 41344_s_at Cluster Incl. M96684:H. sapiens Pur (pur-alpha) mRNA, complete cds /cd; 1316_at X55005 /FEATURE=mRNA /DEFINITION=HSCERBAR Homo sapiens mRNA for thyroid; 1089_i_at M64936 /FEATURE= /DEFINITION=HUMRIRT Homo sapiens retinoic acid-induci; 551_at U01877 /FEATURE= /DEFINITION=HSU01877 Human p300 protein mRNA, complete c; 440_at X12492 /FEATURE=cds /DEFINITION=HSCTF1 Human mRNA for CAAT-box binding tr.

Metagene 58; 35961_at Cluster Incl. AL049390:Homo sapiens mRNA; cDNA DKFZp586O1318 (from clon; 36227_at Cluster Incl. AF043129:untitled /cds=(0,1379) /gb=AF043129 /gi=3978161; 36280_at Cluster Incl. U26174:Human pre-granzyme 3 mRNA, complete cds /cds=(40,8; 37416_at Cluster Incl. Z35227:H. sapiens TTF mRNA for small G protein /cds=(579,1; 37479_at Cluster Incl. M54992:Human B cell differentiation antigen mRNA, complet; 40364_at Cluster Incl. U83460:Human high-affinity copper uptake protein (hCTR1); 40699_at Cluster Incl. M12824:Human T-cell differentiation antigen Leu-2 /T8 mRNA; 40738_at Cluster Incl. M16336:Human T-cell surface antigen CD2 (T11) mRNA, compl; 35698_at Cluster Incl. Y00318:Human mRNA for complement control protein factor I; 36878_f_at Cluster Incl. M60028:Human MHC class II HLA-DQ-beta (DQB1,DQw9), comp; 41764_at Cluster Incl. AA976838:oq35c12.s1 Homo sapiens cDNA, 3 end /clone=IMAG; 34342_s_at Cluster Incl. AF052124:Homo sapiens clone 23810 osteopontin mRNA, com; 34362_at Cluster Incl. M55531:Human glucose transport-like 5 (GLUT5) mRNA, compl; 35260_at Cluster Incl. AB020674:Homo sapiens mRNA for KIAA0867 protein, complete; 33168_at Cluster Incl. H24861:yl42e11.r1 Homo sapiens cDNA, 5 end /clone=IMAGE-; 2092_s_at J04765 /FEATURE= /DEFINITION=HUMOSTRO Human osteopontin mRNA, complete; 1403_s_at M21121 /FEATURE= /DEFINITION=H-UMTCSM Human T cell-specific protein (RA; 964_at X95715 /FEATURE=cds /DEFINITION=HSARAPROT H. sapiens mRNA for anthracyclin; 608_at M12529 /FEATURE=mRNA /DEFINITION=HUMAPOE Human apolipoprotein E mRNA, com.

Metagene 59; 37793_r_at Cluster Incl. AF034956:Homo sapiens RAD51D mRNA, complete cds /cds=(1; 39304_g_at Cluster Incl. Y14153:Homo sapiens mRNA for beta-transducin repeat con; 41624_r_at Cluster Incl. AA932443:oo75b11.s1 Homo sapiens cDNA, 3 end /clone=IM; 35686_s_at Cluster Incl. Z24459:H. sapiens MTCP1 gene, exons 2A to 7 (and joined; 36000_at Cluster Incl. X98054:H. sapiens mRNA for G13 protein /cds=(33,2144) /gb=; 36516_at Cluster Incl. AL080143:Homo sapiens mRNA; cDNA DKFZp434N043 (from clone; 38307_at Cluster Incl. AB011179:Homo sapiens mRNA for KIAA0607 protein, partial; 40058_s_at Cluster Incl. AI341656:qq95e10.x1 Homo sapiens cDNA, 3 end /clone=IM; 40466_at Cluster Incl. Z74792:H. sapiens mRNA for CCAAT transcription binding fac; 40490_at Cluster Incl. U41387:Human Gu protein mRNA, partial cds /cds=(0,2405) /; 36168_at Cluster Incl. X66945:H. sapiens N-sam mRNA for fibroblast growth factor; 38027_at Cluster Incl. X53742:H. sapiens mRNA for fibulin-1 B /cds=(10,1815) /gb=; 41597_s_at Cluster Incl. AF047442:Homo sapiens vesicle trafficking protein sec22; 1910_s_at M14745 /FEATURE=cds /DEFINITION=HUMBCL2C Human bcl-2 mRNA.

Metagene 60; 39683_at Cluster Incl. U35340:Human beta B1-crystallin mRNA, complete cds /cds=(; 35715_at Cluster Incl. AL080071:Homo sapiens mRNA; cDNA DKFZp564M082 (from clone; 36916_at Cluster Incl. X74570:H. sapiens mRNA for Gal-beta(1-3/1-4)GlcNAc alpha-2; 37216_at Cluster Incl. AB023180:Homo sapiens mRNA for KIAA0963 protein, complete; 38333_at Cluster Incl. Y11731:H. sapiens mRNA for DNA glycosylase /cds=(338,1375); 38996_at Cluster Incl. U15655:Human ets domain protein ERF mRNA, complete cds /c; 35851_g_at Cluster Incl. AI950382:wp10g06.x1 Homo sapiens cDNA, 3 end /clone=IM; 815_at U70987 /FEATURE= /DEFINITION=HSU70987 Human GAP binding protein p62dok (D; 754_s_at D87002 /FEATURE=cds#1 /DEFINITION=D87002 Homo sapiens immunoglobulin 1a; 544_at S76638 /FEATURE= /DEFINITION=S76638 p50-NF-kappa B homolog [human, periph; 136_at U65402 /FEATURE=cds /DEFINITION=HSU65402 Human seven transmembrane G-coup.

Metagene 61; 41004_at Cluster Incl. U43431:Human DNA topoisomerase III mRNA, complete cds /cd; 38675_at Cluster Incl. AI087268:oz77e01.x1 Homo sapiens cDNA, 3 end /clone=IMAG; 37767_at Cluster Incl. L12392:Homo sapiens Huntingtons Disease (HD) mRNA, comple; 40547_at Cluster Incl. AI688516:wc90b03.x1 Homo sapiens cDNA, 3 end /clone=IMAG; 543_g_at S74445 /FEATURE= /DEFINITION=S74445 cellular retinoic acid-binding prot.

Metagene 62; 39082_at Cluster Incl. Y00097:Human mRNA for protein p68 /cds=(100,2121) /gb=Y00; 32230_at Cluster Incl. U39067:Homo sapiens translation initiation factor eIF3 p3; 40951_at Cluster Incl. AL049250:Homo sapiens mRNA; cDNA DKFZp564D113 (from clone; 41800_s_at Cluster Incl. U46571:Human tetratricopeptide repeat protein (tpr2) mR; 960_g_at Guanine Nucleotide-Binding Protein G25k; 223_at S81003 /FEATURE= /DEFINITION=S81003 L-UBC=ubiquitin conjugating enzyme [h.

Metagene 63; 32991_f_at Cluster Incl. M86933:Human amelogenin (AMELY) mRNA, complete cds /cds; 38154_at Cluster Incl. AF038199:Homo sapiens clone 23728 mRNA sequence /cds=UNKN; 40020_at Cluster Incl. AB011536:Homo sapiens mRNA for MEGF2, partial cds /cds=(0; 33249_at Cluster Incl. M16801:Human mineralocorticoid receptor mRNA (hMR), compl; 37588_s_at Cluster Incl. U62317:Chromosome 22q13 BAC Clone CIT987SK-384D8 comple; 39050_at Cluster Incl. AF026029:Homo sapiens poly(A) binding protein II (PABP2); 33908_at Cluster Incl. X04366:Human mRNA for calcium activated neutral protease; 38480_s_at Cluster Incl. U66867:Human ubiquitin conjugating enzyme 9 (hUBC9) mRN; 39190_s_at Cluster Incl. AC002126:Homo sapiens DNA from chromosome 19-cosmids R3; 40542_at Cluster Incl. AI793265:oo49c07.y5 Homo sapiens cDNA, 5 end /clone=IMAG; 633_s_at L40386 /FEATURE=mRNA /DEFINITION=HUMDP2M Human DP-2 mRNA, complete cds; 252_at L42324 /FEATURE=cds /DEFINITION=HUMFRCG Homo sapiens (clone GPCR W) G pro.

Metagene 64; 38860_at Cluster Incl. U66346:Homo sapiens cAMP phosphodiesterase (PDE4C) mRNA,; 39582_at Cluster Incl. AL050166:Homo sapiens mRNA; cDNA DKFZp586D1122 (from clon; 35164_at Cluster Incl. AF084481:Homo sapiens transmembrane protein (WFS1) mRNA,; 35710_s_at Cluster Incl. U95006:Human D9 splice variant A mRNA, complete cds /cd; 36523_at Cluster Incl. L06133:Human putative Cu++-transporting P-type ATPase mRN; 38627_at Cluster Incl. M95585:Human hepatic leukemia factor (HLF) mRNA, complete; 39441_at Cluster Incl. Y11395:H. sapiens m-RNA for p40 /cds=(104,1303) /gb=Y11395; 40052_at Cluster Incl. X82206:H. sapiens mRNA for alpha-centractin /cds=(66,1196); 40447_at Cluster Incl. D87436:Human mRNA for KIAA0249 gene, complete cds /cds=(2; 40816_at Cluster Incl. L07758:Human IEF SSP 9502 mRNA, complete cds /cds=(87,159; 34338_at Cluster Incl. D49738:Human cytoskeleton associated protein (CG22) mRNA,; 34348_at Cluster Incl. U78095:Homo sapiens placental bikunin mRNA, complete cds; 35318_at Cluster Incl. AB007944:Homo sapiens mRNA for KIAA0475 protein, complete; 36667_at Cluster Incl. U47025:Human fetal brain glycogen phosphorylase B mRNA, c; 37293_at Cluster Incl. D43948:Human mRNA for KIAA0097 gene, complete cds /cds=(4; 37362_at Cluster Incl. X54871:H. sapiens mRNA for ras-related protein Rab5b /cds=; 38842_at Cluster Incl. AB023206:Homo sapiens mRNA for KIAA0989 protein, partial; 743_at D50370 /FEATURE= /DEFINITION=D50370 Homo sapiens mRNA for nucleosome asse.

Metagene 65; 32378_at Cluster Incl. M26252:Human TCB gene encoding cytosolic thyroid hormone-; 38945_at Cluster Incl. X78710:H. sapiens MTF-1 mRNA for metal-regulatory transcri; 40332_at Cluster Incl. AF109134:Homo sapiens 7-60 mRNA, complete cds /cds=(205,2; 34707_at Cluster Incl. U91543:Homo sapiens zinc-finger helicase (hZFH) mRNA, com; 38998_g_at Cluster Incl. X96924:H. sapiens gene encoding mitochondrial citrate tr; 39722_at Cluster Incl. AF044209:Homo sapiens nuclear receptor co-repressor N-CoR; 40143_at Cluster Incl. D50930:Human mRNA for KIAA0140 gene, complete cds /cds=(2; 40147_at Cluster Incl. U18009:Human chromosome 17q21 mRNA clone LF113 /cds=(0,93; 40514_at Cluster Incl. AF091085:Homo sapiens clone 638 unknown mRNA, complete se; 40791_at Cluster Incl. X63564:H. sapiens mRNA for RNA polymerase II largest subun; 41724_at Cluster Incl. X81817:H. sapiens BAP31 mRNA /cds=(73,813) /gb=X81817 /gi=; 34874_at Cluster Incl. AJ004832:Homo sapiens mRNA for neuropathy target esterase; 38483_at Cluster Incl. AJ011916:Homo sapiens mRNA for hypothetical protein /cds=; 38789_at Cluster Incl. L12711:Homo sapiens transketolase (tk) mRNA, complete cds; 38790_at Cluster Incl. L25879:Homo sapiens p53 /HEH epoxide hydrolase (EPHX) mRNA; 39112_at Cluster Incl. Y07661:H. sapiens USF2 gene /cds=(0,1040) /gb=Y07661 /gi=1; 39155_at Cluster Incl. D67025:Homo sapiens mRNA for proteasome subunit p58, comp; 39825_at Cluster Incl. L77567:Homo sapiens mitochondrial citrate transport prote; 39833_at Cluster Incl. R54564:yg81b12.s1 Homo sapiens cDNA, 3 end /clone=IMAGE-; 1268_at M58028 /FEATURE=mRNA /DEFINITION=HUMUBIQAA Human ubiquitin-activating en; 1170_at Colony-Stimulating Factor 1, Macrophage, Alt. Splice 3; 605_at L78833 /FEATURE=exon#36 /DEFINITION=HUMBRCA1 Human BRCA1, Rho7 and vatI g.

Metagene 66; 35531_at Cluster Incl. AB007933:Homo sapiens mRNA for KIAA0464 protein, complete; 37136_at Cluster Incl. AB000520:Homo sapiens mRNA for APS, complete cds /cds=(12; 33733_at Cluster Incl. AF093771:Homo sapiens mitoxantrone resistance protein 1 m; 34260_at Cluster Incl. AB014583:Homo sapiens mRNA for KIAA0683 protein, complete; 38641_at Cluster Incl. AJ133115:Homo sapiens mRNA for TSC-22-like protein /cds=(; 39332_at Cluster Incl. AF035316:Homo sapiens clone 23678 mRNA, partial cds /cds=; 40444_s_at Cluster Incl. AB002382:Human mRNA for KIAA0384 gene, complete cds /cd; 36583_at Cluster Incl. U53225:Human sorting nexin 1 (SNX1) mRNA, complete cds /c; 37005_at Cluster Incl. D28124:Human mRNA for unknown product, complete cds /cds=; 37400_at Cluster Incl. J03068:Human DNF1552 (lung) mRNA, complete cds /cds=(292,; 37744_r_at Cluster Incl. U60062:Human FEZ1-T mRNA, alternatively spliced form, c; 38398_at Cluster Incl. AB002356:Human mRNA for KIAA0358 gene, complete cds /cds=; 1587_at M38258 /FEATURE= /DEFINITION=HUMRARGA Human retinoic acid receptor gamma; 1401_g_at M13207 /FEATURE=expanded_cds /DEFINITION=HUMCSFGMA Human granulocyte-m; 1019_g_at U81787 /FEATURE= /DEFINITION=HSU81787 Human Wnt10B mRNA, complete cds; 858_at S90469 /FEATURE= /DEFINITION=S90469 cytochrome P450 reductase [human, pla; 679_at J04990 /FEATURE=cds /DEFINITION=HUMCAPG Human cathepsin G gene, complete; 495_at U31628 /FEATURE= /DEFINITION=HSU31628 Human interleukin-15 receptor alpha.

Metagene 67; 34491_at Cluster Incl. AJ225089:Homo sapiens mRNA for 2-5 oligoadenylate synthe; 35633_at Cluster Incl. D87457:Human mRNA for KIAA0281 gene, complete cds /cds=(1; 37545_at Cluster Incl. W22110:64F11 Homo sapiens cDNA /clone=(not-directional) /; 37630_at Cluster Incl. AL049176:Human DNA sequence from clone 141H5 on chromosom; 36680_at Cluster Incl. M24895:Homo sapiens alpha-amylase mRNA, complete cds /cds; 37695_at Cluster Incl. D79983:Human mRNA for KIAA0161 gene, complete cds /cds=(3; 1708_at U07620 /FEATURE= /DEFINITION=HSU07620 Human MAP kinase mRNA, complete cd; 1389_at J03779 /FEATURE-mRNA /DEFINITION=HUMCALLA Human common acute lymphoblast; 753_at D86425 /FEATURE= /DEFINITION=D86425 Homo sapiens mRNA for osteonidogen, c; 160029_at X07109 /FEATURE=cds /DEFINITION=HSPKCB2A Human mRNA for protein kinase.

Metagene 68; 41087_at Cluster Incl. AC004774:Homo sapiens BAC clone RG300E22 from 7q21-q31.1; 37560_at Cluster Incl. AL050143:Homo sapiens mRNA; cDNA DKFZp586B2420 (from clon; 32154_at Cluster Incl. M36711:Human sequence-specific DNA-binding protein (AP-2); 1177_at Dna-Binding Protein Ap-2, Alt. Splice 3; 235_at M59488 /FEATURE=mRNA /DEFINITION=HUMS100B3 Human S100 protein beta-subuni.

Metagene 69; 31504_at Cluster Incl. M64098:Human high density lipoprotein binding protein (HB; 39258_at Cluster Incl. AI627877:ty20b09.x1 Homo sapiens cDNA, 3 end /clone=IMAG; 41670_at Cluster Incl. R38263:yc92c11.s1 Homo sapiens cDNA, 3 end /clone=IMAGE-; 34255_at Cluster Incl. AF059202:Homo sapiens ACAT related gene product 1 mRNA, c; 35979_at Cluster Incl. AF081287:Homo sapiens serine phosphatase FCP1a (FCP1) mRN; 38612_at Cluster Incl. M69023:Human globin gene /cds=UNKNOWN /gb=M69023 /gi=1831; 39003_at Cluster Incl. Z50022:H. sapiens mRNA for surface glycoprotein /cds=(93,6; 39064_at Cluster Incl. L38928:Homo sapiens 5,10-methenyltetrahydrofolate synthet; 40087_at Cluster Incl. AF020202:Homo sapiens Munc13 mRNA, complete cds /cds=(224; 40090_at Cluster Incl. AI797997:wh80b02.x1 Homo sapiens cDNA, 3 end /clone=IMAG; 40134_at Cluster Incl. AF047436:Homo sapiens F1Fo-ATPase synthase f subunit mRNA; 40882_at Cluster Incl. Y09616:H. sapiens mRNA for putative carboxylesterase /cds=; 41223_at Cluster Incl. M22760:Homo sapiens nuclear-encoded mitochondrial cytochr; 33424_at Cluster Incl. Y00281:Human mRNA for ribophorin I /cds=(137,1960) /gb=Y0; 35322_at Cluster Incl. D50922:Human mRNA for KIAA0132 gene, complete cds /cds=(1; 36189_at Cluster Incl. U10323:Human nuclear factor NF45 mRNA, complete cds /cds=; 36951_at Cluster Incl. U39400:Human NOF1 mRNA, complete cds /cds=(13,513) /gb=U3; 37361_at Cluster Incl. AF010187:Homo sapiens FGF-1 intracellular binding protein; 37730_at Cluster Incl. U22055:Human 100 kDa coactivator mRNA, complete cds /cds=; 37751_at Cluster Incl. D87444:Human mRNA for KIAA0255 gene, complete cds /cds=(3.

Metagene 70; 31532_at Cluster Incl. U43292:Human MDS1B (MDS1) mRNA, complete cds /cds=(307,81; 33683_at Cluster Incl. D50525:Human mRNA for TI-227H /cds=UNKNOWN /gb=D50525 /gi; 41046_s_at Cluster Incl. X95808:H. sapiens mRNA for protein encoded by a candidat; 41604_at Cluster Incl. U79297:Human clone 23589 mRNA sequence /cds=UNKNOWN /gb=U; 32623_at Cluster Incl. AJ225028:Homo sapiens mRNA for GABA-B R1a receptor /cds=(; 34276_at Cluster Incl. AB023197:Homo sapiens mRNA for KIAA0980 protein, partial; 37592_at Cluster Incl. J05401:Human sarcomeric mitochondrial creatine kinase (Mt; 38664_at Cluster Incl. AB009285:Homo sapiens BCNT mRNA, complete cds /cds=(109,1; 40057_at Cluster Incl. U94777:Human muscle glycogen phosphorylase (PYGM) gene, 5; 40779_at Cluster Incl. U59919:Human 5 mg GDS-associated protein SMAP mRNA, comple; 33880_at Cluster Incl. D89053:Homo sapiens mRNA for Acyl-CoA synthetase 3, compl; 33881_at Cluster Incl. AA977580:on61b02.s1 Homo sapiens cDNA, 3 end /clone=IMAG; 37683_at Cluster Incl. D80012:Human mRNA for KIAA0190 gene, partial cds /cds=(0,; 39123_s_at Cluster Incl. X89066:H. sapiens mRNA for TRPC1 protein /cds=(137,2416); 1229_at U78556 /FEATURE= /DEFINITION=HSU78556 Human cisplatin resistance associa.

Metagene 71; 32468_f_at Cluster Incl. D90278:Human CGM1b mRNA for CD66d /cds=(61,594) /gb=D90; 32265_at Cluster Incl. D85245:Homo sapiens mRNA for TR3beta, complete cds /cds=(; 39596_at Cluster Incl. U05012:Human receptor tyrosine kinase TrkC (NTRK3) mRNA,; 33740_at Cluster Incl. AF023268:Homo sapiens clk2 kinase (CLK2), propin1, cote1,; 38658_at Cluster Incl. X81636:H. sapiens clathrin light chain a gene /cds=UNKNOWN; 39164_at Cluster Incl. AF099149:Homo sapiens TRIAD1 type I mRNA, complete cds /c; 1059_at S76475 /FEATURE= /DEFINITION=S76475 trkC [human, brain, mRNA, 2715 nt]; 1060_g_at S76475 /FEATURE= /DEFINITION=S76475 trkC [human, brain, mRNA, 2715 nt].

Metagene 72; 32901_s_at Cluster Incl. AC005192:Homo sapiens BAC clone RG163K11 from 7q31 /cds; 32134_at Cluster Incl. AL050162:Homo sapiens mRNA; cDNA DKFZp586B2022 (from clon; 33240_at Cluster Incl. AB029018:Homo sapiens mRNA for KIAA1095 protein, partial; 36535_at Cluster Incl. U04209:Human associated microfibrillar protein mRNA, comp; 36814_at Cluster Incl. AB029032:Homo sapiens mRNA for KIAA1109 protein, partial; 38690_at Cluster Incl. AL080097:Homo sapiens mRNA; cDNA DKFZp564P0462 (from clon; 39072_at Cluster Incl. L07648:Human MXI mRNA, complete cds /cds=(208,894) /gb=L; 39790_at Cluster Incl. M23115:Homo sapiens calcium-ATPase (HK2) mRNA, complete c; 39797_at Cluster Incl. AB002347:Human mRNA for KIAA0349 gene, partial cds /cds=(; 40140_at Cluster Incl. D76444:Homo sapiens hkf-1 mRNA, complete cds /cds=(922,29; 41191_at Cluster Incl. AB023209:Homo sapiens mRNA for KIAA0992 protein, partial; 32780_at Cluster Incl. AB018271:Homo sapiens mRNA for KLAA0728 protein, partial; 32827_at Cluster Incl. AI365215:qz41a06.x1 Homo sapiens cDNA, 3 end /clone=IMAG; 32847_at Cluster Incl. U48959:Homo sapiens myosin light chain kinase (MLCK) mRNA; 33378_at Cluster Incl. AB019494:Homo sapiens IDN3 mRNA, partial cds /cds=(706,75; 33442_at Cluster Incl. AB002365:Human mRNA for KIAA0367 gene, partial cds /cds=(; 33865_at Cluster Incl. AA127624:zk89b09.r1 Homo sapiens cDNA, 5 end /clone=IMAG; 35255_at Cluster Incl. AF098799:Homo sapiens RanBP7 /importin 7 mRNA, complete cd; 35279_at Cluster Incl. U33821:Human tax1-binding protein TXBP151 mRNA, complete; 36620_at Cluster Incl. X02317:Human mRNA for Cu/Zn superoxide dismutase (SOD) /c; 36627_at Cluster Incl. X86693:H. sapiens mRNA for hevin like protein /cds=(322,23; 37698_at Cluster Incl. X97335:H. sapiens mRNA for kinase A anchor protein /cds=(1; 38047_at Cluster Incl. D84109:Homo sapiens mRNA for RBP-MS/type 3, complete cds; 38120_at Cluster Incl. U50928:Human autosomal dominant polycystic kidney disease; 38375_at Cluster Incl. AF112219:Homo sapiens esterase D mRNA, complete cds /cds=; 38443_at Cluster Incl. U79291:Human clone 23721 mRNA sequence /cds=UNKNOWN /gb=U; 38768_at Cluster Incl. X96752:H. sapiens mRNA for L-3-hydroxyacyl-CoA dehydrogena; 39163_at Cluster Incl. W27233:24b7 Homo sapiens cDNA /gb=W27233 /gi=1306749 /ug=; 39838_at Cluster Incl. AB014522:Homo sapiens mRNA for KIAA0622 protein, partial; 39852_at Cluster Incl. AB011182:Homo sapiens mRNA for KIAA0610 protein, partial ; 40610_at Cluster Incl. AI743507:wf72a06.x2 Homo sapiens cDNA, 3 end /clone=IMAG; 41000_at Cluster Incl. U68723:Human checkpoint suppressor 1 mRNA, complete cds /; 41277_at Cluster Incl. AW021542:df25a06.y1 Homo sapiens cDNA, 5 end /clone=IMAG; 41289_at Cluster Incl. AA126505:zn86a09.s1 Homo sapiens cDNA, 3 end /clone=IMAG; 41795_at Cluster Incl. X17576:Human melanoma mRNA for nck protein, showing homo1; 41815_at Cluster Incl. AL080133:Homo sapiens mRNA; cDNA DKFZp434G173 (from clone; 32542_at Cluster Incl. AF063002:Homo sapiens LIM protein SLIMMER mRNA, complete; 1725_s_at Oncogene E6-Ap, Papillomavirus; 1327_s_at U67156 /FEATURE= /DEFINITION=HSU67156 Human mitogen-activated kinase k; 1278_at Tyrosine Kinase, Receptor Axl, Alt. Splice 2; 654_at L07648 /FEATURE= /DEFINITION=HUMMXI1A Human MXI1 mRNA, complete cds; 409_at X56468 /FEATURE=mRNA /DEFINITION=HS1433 Human mRNA for 14.3.3 protein, a; 171_at U56833 /FEATURE= /DEFINITION=HSU56833 Human VHL binding protein-1 (VBP-1).

Metagene 73; 34033_s_at Cluster Incl. AF025531:Homo sapiens leucocyte immunoglobulin-like rec; 40019_at Cluster Incl. M60830:Human EVI2B3P gene, exon and complete cds /cds=(21; 34660_at Cluster Incl. A1142565:qb47d08.x1 Homo sapiens cDNA, 3 end /clone=IMAG; 37541_at Cluster Incl. U25956:Human P-selectin glycoprotein ligand (SELPLG) gene; 37598_at Cluster Incl. D79990:Human mRNA for KIAA0168 gene, complete cds /cds=(1; 38973_at Cluster Incl. AB028943:Homo sapiens mRNA for KIAA1020 protein, partial.

Metagene 74; 31627_f_at Cluster Incl. AF047485:Homo sapiens amine oxidase pseudogene mRNA, sp; 31988_at Cluster Incl. AF043468:Homo sapiens neurexophilin 3 (NPH3) mRNA, partia; 33014_at Cluster Incl. AF059194:Homo sapiens basic-leucine zipper transcription; 36369_at Cluster Incl. AF000421:Homo sapiens TTF-I interacting peptide 12 mRNA,; 34533_at Cluster Incl. AF038192:Homo sapiens clone 23808 mRNA sequence /cds=UNKN; 36235_at Cluster Incl. W26334:26b1 Homo sapiens cDNA /gb=W26334 /gi=1306889 /ug=; 37111_g_at Cluster Incl. AB012229:Homo sapiens gene for fructose-6-phosphate, 2-k; 32063_at Cluster Incl. M86546:H. sapiens PBX1a and PBX1b mRNA, complete cds /cds=; 32098_at Cluster Incl. M20777:Homo sapiens, alpha-2 (VI) collagen /cds=UNKNOWN /; 32698_at Cluster Incl. X64116:H. sapiens PVR gene for poliovirus receptor (exon 1; 34725_at Cluster Incl. M73077:Human glucocorticoid receptor repression factor 1; 38002_s_at Cluster Incl. Z34822:H. sapiens (HLCC85) mRNA for voltage-dependent L-; 40060_r_at Cluster Incl. AF061258:Homo sapiens LIM protein mRNA, complete cds /c; 40100_at Cluster Incl. U72206:Human guanine nucleotide regulatory factor (LFP40); 34374_g_at Cluster Incl. Z97054:Human DNA sequence from PAC 339A18 on chromosome; 37327_at Cluster Incl. X00588:Human mRNA for precursor of epidermal growth facto; 38844_at Cluster Incl. L42451:Homo sapiens pyruvate dehydrogenase kinase isoenzy; 39522_at Cluster Incl. D49817:Homo sapiens mRNA for 6-phosphofructo-2-kinase/fru; 40913_at Cluster Incl. W28589:48h12 Homo sapiens cDNA /gb=W28589 /gi=1308537 /ug; 41285_at Cluster Incl. X77567:H. sapiens mRNA for InsP3 5-phosphatase /cds=(99,13; 41350_at Cluster Incl. M20776:Homo sapiens, alpha-1 (VI) collagen /cds=UNKNOWN /; 41550_at Cluster Incl. AF091071:Homo sapiens clone 192 Rer1 mRNA, complete cds /; 2040_s_at M14752 /FEATURE= /DEFINITION=HUMABLA Human c-abl gene, complete cds; 1938_at K03218 /FEATURE=cds /DEFINITION=HUMSRC11 Human c-src-1 proto-oncogene, e; 1896_s_at L05628 /FEATURE= /DEFINITION=HUMMRPX Human multidrug resistance-associ; 1901_s_at M12036 /FEATURE=cds /DEFINITION=HUMHER2B Human tyrosine kinase-type re; 1221_at X54871 /FEATURE=cds /DEFINITION=HSRAB5B H. sapiens mRNA for ras-related p; 1045_s_at U33838 /FEATURE= /DEFINITION=HSU33838 Human NF-kappa-B p65delta3 mRNA,; 773_at D10667 /FEATURE= /DEFINITION=HUMMHCAAA Homo sapiens mRNA for smooth muscl; 774_g_at D10667 /FEATURE= /DEFINITION=HUMMHCAAA Homo sapiens mRNA for smooth mus.

Metagene 75; 36790_at Cluster Incl. M19267:Human tropomyosin mRNA, complete cds /cds=(286,114; 38590_r_at Cluster Incl. M14630:Human prothymosin alpha mRNA, complete cds /cds=; 32700_at Cluster Incl. M55543:Human guanylate binding protein isoform II (GBP-2); 34720_at Cluster Incl. U85193:Human nuclear factor I-B2 (NFIB2) mRNA, complete c; 36918_at Cluster Incl. Y15723:Homo sapiens mRNA for soluble guanylyl cyclase /cd; 39000_at Cluster Incl. AF043324:Homo sapiens N-myristoyltransferase 1 mRNA, comp; 33890_at Cluster Incl. AB008109:Homo sapiens mRNA for RGS5, complete cds /cds=(8; 34811_at Cluster Incl. U09813:Human mitochondrial ATP synthase subunit 9, P3 gen; 32544_s_at Cluster Incl. L12535:Human RSU-1/RSP-1 mRNA, complete cds /cds=(827,1; 1772_s_at L00634 /FEATURE= /DEFINITION=HUMFPTA Human farnesyl-protein transferas.

Metagene 76; 37243_at Cluster Incl. X66533:H. sapiens soluble guanylate cyclase small subunit; 39431_at Cluster Incl. AJ132583:Homo sapiens mRNA for puromycin sensitive aminop; 38439_at Cluster Incl. L24123:Homo sapiens NRF1 protein (NRF1) mRNA /cds=UNKNOWN; 41841_at Cluster Incl. AF052138:Homo sapiens clone 23718 mRNA sequence /cds=UNKN; 1814_at D50683 /FEATURE= /DEFINITION=D50683 Homo sapiens mRNA for TGF-betaIIR al.

Metagene 77; 33090_at Cluster Incl. AJ007292:Homo sapiens mRNA for ephrin-A2 /cds=(15,656) /g; 40029_at Cluster Incl. AB011539:Homo sapiens mRNA for MEGF6, partial cds /cds=(0; 35347_at Cluster Incl. AF093119:Homo sapiens UPH1 (UPH1) mRNA, complete cds /cds; 1109_s_at M19989 /FEATURE=cds /DEFINITION=HUMPDGFA7 Human platelet-derived growt.

Metagene 78; 31638_at Cluster Incl. AC005329:Homo sapiens chromosome 19, cosmid R34382 /cds=(; 33386_at Cluster Incl. Z97630:Human DNA sequence from clone 466N1 on chromosome; 38458_at Cluster Incl. L39945:Human cytochrome b5 (CYB5) gene /cds=(120,548) /gb; 1645_at U43527 /FEATURE= /DEFINITION=HSU43527 Human malignant melanoma metastasi; 751_at D85418 /FEATURE= /DEFINITION=D85418 Homo sapiens mRNA for phosphatidylino; 286_at L19779 /FEATURE= /DEFINITION=HUMH2A2A Homo sapiens histone H2A.2 mRNA, co.

Metagene 79; 35512_at Cluster Incl. AC005764:Homo sapiens chromosome 19, cosmid R31343 /cds=(; 33545_at Cluster Incl. M81758:Homo sapiens skeletal muscle voltage-dependent sod; 39620_at Cluster Incl. Z21966:H. sapiens mPOU homeobox protein mRNA /cds=(192,109; 41652_at Cluster Incl. AL031228:dJ1033B10.12 (collagen, type XI, alpha 2 (COL11A; 33792_at Cluster Incl. AF043498:Homo sapiens prostate stem cell antigen (PSCA) m; 38012_at Cluster Incl. U03272:Human fibrillin-2 mRNA, complete cds /cds=(0,8735); 1153_f_at J00117 /FEATURE=mRNA /DEFINITION=HUMCGB Human chorionic gonadotropin (; 697_f_at Luteinizing Hormone, Beta Subunit; 701_s_at Insulin-Like Leydig Hormone.

Metagene 80; 36804_at Cluster Incl. M34455:Human interferon-gamma-inducible indoleamine 2,3-d; 37467_at Cluster Incl. K02882:Human germline IgD chain gene, C-region, C-delta-1; 34799_at Cluster Incl. AF070643:Homo sapiens clone 24636 mRNA sequence /cds=UNKN; 36205_at Cluster Incl. L04490:Homo sapiens (clone CC6) NADH-ubiquinone oxidoredu; 634_at L41351 /FEATURE=mRNA /DEFINITION=HUMPROS Homo sapiens prostasin mRNA, com.

Metagene 81; 31991_at Cluster Incl. AL049430:Homo sapiens mRNA; cDNA DKFZp586H201 (from clone; 33011_at Cluster Incl. Y10148:H. sapiens mRNA for NTR2 receptor /cds=(36,1268) /g; 34108_g_at Cluster Incl. AJ005577:Homo sapiens pfkfb2 gene, exons 1 to 15 /cds=(; 34131_at Cluster Incl. AB026891:Homo sapiens mRNA for cystine/glutamate transpor; 34933_at Cluster Incl. AJ238381:Homo sapiens pax9 gene, exons 1-2 and joined CDS; 36237_at Cluster Incl. AB009698:Homo sapiens mRNA for hOAT1-2, complete cds /cds; 38161_at Cluster Incl. Y09022:H. sapiens mRNA for Not56-like protein /cds=(31,134; 40402_at Cluster Incl. X91117:H. sapiens HG NET gene exon 1 /cds=(49,1902) /gb=X9; 41443_at Cluster Incl. U63127:Human SEC7 homolog Tic (TIC) mRNA, complete cds /c; 32028_at Cluster Incl. U85773:Human phosphomannomutase (PMM2) mRNA, complete cds; 32031_at Cluster Incl. D78586:Human CAD mRNA for multifunctional protein CAD, co; 33224_at Cluster Incl. AB007965:Homo sapiens mRNA, chromosome 1 specific transcr; 33281_at Cluster Incl. D63485:Human mRNA for KIAA0151 gene, complete cds /cds=(3; 38297_at Cluster Incl. X98654:H. sapiens mRNA for DRES9 protein /cds=(189,3923) /; 38710_at Cluster Incl. AL096714:Homo sapiens mRNA; cDNA DKFZp564E242 (from clone; 39010_at Cluster Incl. AI658639:tu06g05.x1 Homo sapiens cDNA, 3 end /clone=IMAG; 39339_at Cluster Incl. AB018335:Homo sapiens mRNA for KIAA0792 protein, complete; 40414_at Cluster Incl. X59303:Human G7a mRNA for valyl-tRNA synthetase /cds=(219; 33360_at Cluster Incl. AB023221:Homo sapiens mRNA for KIAA1004 protein, partial; 33842_at Cluster Incl. AC004472:Homo sapiens chromosome 9, P1 clone 11659 /cds=(; 36670_at Cluster Incl. L26339:Human autoantigen mRNA, complete cds /cds=(136,378; 40176_at Cluster Incl. J03407:Human rfp transforming protein mRNA, complete cds; 40890_at Cluster Incl. U46920:Human metaxin (MTX) gene, complete cds /cds=(0,953; 41804_at Cluster Incl. W80358:zh49a07.s1 Homo sapiens cDNA, 3 end /clone=IMAGE-; 1294_at L13852 /FEATURE= /DEFINITION=HUMEIURP Homo sapiens ubiquitin-activating; 1003_s_at X68149 /FEATURE=cds /DEFINITION=HSBLRIA Homo sapiens BLR1 gene for Bur; 686_s_at K03498 /FEATURE=cds#1 /DEFINITION=HUMERVDP Human endogenous retrovirus; 567_s_at M79463 /FEATURE= /DEFINITION=HUMPML2 Human PML-2 mRNA, complete CDS.

Metagene 82; 33082_at Cluster Incl. AF074015:Homo sapiens integrin subunit alpha 10 precursor; 35592_at Cluster Incl. AB029010:Homo sapiens mRNA for KIAA1087 protein, partial; 36364_at Cluster Incl. L32961:Human 4-aminobutyrate aminotransferase (GABAT) mRN; 33464_at Cluster Incl. AL109703:Homo sapiens mRNA fall length insert cDNA clone; 41406_at Cluster Incl. AL080172:Homo sapiens mRNA; cDNA DKFZp434G231 (from clone.

Metagene 83; 31347_at Cluster Incl. AF058075:Homo sapiens clone ASPBLL54 immunoglobulin lambd; 31512_at Cluster Incl. Z00010:H. sapiens germ line pseudogene for immunoglobulin; 32904_at Cluster Incl. M28393:Human perforin mRNA, complete cds /cds=(0,1667) /g; 32967_at Cluster Incl. AF057557:Homo sapiens anti-Fas-induced apoptosis (TOSO) m; 35228_at Cluster Incl. Y08682:H. sapiens mRNA for carnitine palmitoyltransferase; 32793_at Cluster Incl. X00437:Human mRNA for T-cell specific protein /cds=(37,97; 38096_f_at Cluster Incl. M83664:Human MHC class II lymphocyte antigen (HLA-DP) b.

Metagene 84; 34988_at Cluster Incl. AF052119:Homo sapiens clone 23622 mRNA sequence /cds=UNKN; 35435_s_at Cluster Incl. AF001903:Human 3-hydroxyacyl-CoA dehydrogenase, isoform; 41111_at Cluster Incl. U68418:Human branched chain aminotransferase precursor (B; 41401_at Cluster Incl. U57646:Homo sapiens cysteine and glycine-rich protein 2 (; 33301_g_at Cluster Incl. AL031282:dJ283E3.3.1 (Cell Division Cycle 2-Like 2 (PIT; 33706_at Cluster Incl. AB006198:Homo sapiens mRNA for SART-1, complete cds /cds=; 39391_at Cluster Incl. AF052135:Homo sapiens clone 23625 mRNA sequence /cds=UNKN; 39427_at Cluster Incl. T79616:yd71e06.s1 Homo sapiens cDNA, 3 end /clone=IMAGE-; 41145_at Cluster Incl. AB020721:Homo sapiens mRNA for KIAA0914 protein, complete; 36586_at Cluster Incl. U78521:Homo sapiens immunophilin homolog ARA9 mRNA, compl; 38078_at Cluster Incl. AF042166:Homo sapiens beta-filamin mRNA, complete cds /cd; 39552_at Cluster Incl. U92436:Human mutated in multiple advanced cancers protein; 41339_at Cluster Incl. AF043117:Homo sapiens ubiquitin-fusion degradation protei; 41524_at Cluster Incl. L08488:Human inositol polyphosphate 1-phosphatase mRNA, c; 1836_at D50310 /FEATURE= /DEFINITION=HUMCYI Human mRNA for cyclin I, complete cd; 1606_at L36645 /FEATURE=mRNA /DEFINITION=HUMRPTKC Homo sapiens receptor protein-; 1348_s_at S79219 /FEATURE= /DEFINITION=S79219 metastasis-associated gene [human,; 1103_at M11567 /FEATURE=mRNA /DEFINITION=HUMAGG Human angiogenin gene, complete.

Metagene 85; 34995_at Cluster Incl. L76380:Homo sapiens (clone HSNME29) CGRP type 1 receptor; 38584_at Cluster Incl. AF026939:Homo sapiens CIG49 (cig49) mRNA, complete cds /c; 38854_at Cluster Incl. AB014535:Homo sapiens mRNA for KIAA0635 protein, complete; 39641_at Cluster Incl. X52486:Human mRNA for uracil-DNA glycosylase /cds=(79,105; 39945_at Cluster Incl. U09278:Human fibroblast activation protein mRNA, complete; 40398_s_at Cluster Incl. AI743406:wg92g12.x1 Homo sapiens cDNA, 3 end /clone=IM; 40716_at Cluster Incl. AL049274:Homo sapiens mRNA; cDNA DKFZp564H203 (from clone; 41058_g_at Cluster Incl. AI760162:wg58e09.x1 Homo sapiens cDNA, 3 end /clone=IM; 34748_at Cluster Incl. AB020653:Homo sapiens mRNA for KIAA0846 protein, complete; 32859_at Cluster Incl. M97935:Homo sapiens transcription factor ISGF-3 mRNA, com; 35362_at Cluster Incl. AB018342:Homo sapiens mRNA for KIAA0799 protein, partial; 35766_at Cluster Incl. M26326:Human keratin 18 mRNA, complete cds /cds=(51,1343); 36181_at Cluster Incl. X82456:H. sapiens MLN50 mRNA /cds=(75,860) /gb=X82456 /gi=; 38432_at Cluster Incl. AA203213:zx57e04.r1 Homo sapiens cDNA, 5 end /clone=IMAG; 1451_s_at D13666 /FEATURE= /DEFINITION=HUMOSF2OS Homo sapiens osf-2 mRNA for ost; 1379_at M59371 /FEATURE=mRNA /DEFINITION=HUMECK Human protein tyrosine kinase mR; 718_at D87258 /FEATURE= /DEFINITION=D87258 Homo sapiens mRNA for serin protease; 719_g_at D87258 /FEATURE= /DEFINITION=D87258 Homo sapiens mRNA for serin proteas.

Metagene 86; 41159_at Cluster Incl. D21260:Human mRNA for KIAA0034 gene, complete cds /cds=(1; 32156_at Cluster Incl. AF044968:untitled /cds=(0,1351) /gb=AF044968 /gi=3941380; 39126_at Cluster Incl. AL080101:Homo sapiens mRNA; cDNA DKFZp564L0472 (from clon; 1719_at U61981 /FEATURE= /DEFINITION=HSU61981 Human putative mismatch repair/bin.

Metagene 87; 32003_at Cluster Incl. D49357:Human mRNA for S-adenosylmethionine synthetase, co; 34171_at Cluster Incl. AI867349:wi24g10.x1 Homo sapiens cDNA, 3 end /clone=IMAG; 39262_at Cluster Incl. U79266:Human clone 23627 mRNA, complete cds /cds=(184,104; 41065_at Cluster Incl. L40407:Homo sapiens thyroid receptor interactor (TRIP9) g; 37911_at Cluster Incl. U07158:Human syntaxin mRNA, complete cds /cds=(66,959) /g; 38647_at Cluster Incl. AJ131182:Homo sapiens mRNA for Epsilon COP /cds=(42,968); 38970_s_at Cluster Incl. AJ011896:Homo sapiens mRNA for HIV-1, Nef-associated fa; 39704_s_at Cluster Incl. L17131:Human high mobility group protein (HMG-I(Y)) gen; 40164_at Cluster Incl. X69550:H. sapiens mRNA for rho GDP-dissociation Inhibitor; 41176_at Cluster Incl. AF052162:Homo sapiens clone 24655 mRNA sequence /cds=UNKN; 33887_at Cluster Incl. D84064:Homo sapiens mRNA for Hrs, complete cds /cds=(60,2; 34780_at Cluster Incl. AB002313:Human mRNA for KIAA0315 gene, partial cds /cds=(; 36187_at Cluster Incl. X13973:Human mRNA for ribonuclease/angiogenin inhibitor (; 37690_at Cluster Incl. U61263:Human acetolactate synthase homolog mRNA, complete; 38791_at Cluster Incl. D29643:Human mRNA for KIAA0115 gene, complete cds /cds=(1; 39134_at Cluster Incl. AJ006973:Homo sapiens mRNA for TOM1 protein /cds=(61,1539; 39812_at Cluster Incl. X79865:H. sapiens Mrp17 mRNA /cds=(137,733) /gb=X79865 /gi; 32559_s_at Cluster Incl. AJ238096:Homo sapiens mRNA for Lsm4 protein /cds=(23,44; 33133_at Cluster Incl. U80184:Homo sapiens FLII gene, complete cds /cds=(35,3844; 33212_at Cluster Incl. AF006751:Homo sapiens ES/130 mRNA, complete cds /cds=(70,; 1942_s_at U37022 /FEATURE=mRNA /DEFINITION=HSU37022 Human cyclin-dependent kinas; 1243_at U18300 /FEATURE= /DEFINITION=HSU18300 Human damage-specific DNA binding; 1100_at L76191 /FEATURE=mRNA /DEFINITION=HUMI1R Homo sapiens interleukin-1 recep; 545_g_at S76638 /FEATURE= /DEFINITION=S76638 p50-NF-kappa B homolog [human, peri; 180_at S82470 /FEATURE= /DEFINITION=S82470 BB1=malignant cell expression-enhance; 151_s_at V00599 /FEATURE=mRNA /DEFINITION=HSTUB2 Human mRNA fragment encoding be.

Metagene 88; 31402_at Cluster Incl. AF103884:Homo sapiens minor histocompatibility antigen HB; 31601_s_at Cluster Incl. U36610:Human Y-chromosome RNA recognition motif protein; 31673_s_at Cluster Incl. X65784:H. sapiens CAR gene /cds=(0,428) /gb=X65784 /gi=4; 35563_at Cluster Incl. AL022238:dJ1042K10.5 (novel protein) /cds=(0,627) /gb=AL0; 32362_r_at Cluster Incl. D87012:Human (lambda) DNA for immunoglobin light chain; 34966_at Cluster Incl. AJ001699:Homo sapiens mRNA for Brachyury (T) protein /cds; 35917_at Cluster Incl. W26631:34a8 Homo sapiens cDNA /gb=W26631 /gi=1307474 /ug=; 37130_g_at Cluster Incl. AI936758:wp69b11.x1 Homo sapiens cDNA, 3 end /clone=IM; 37413_at Cluster Incl. J05257:Homo sapiens (clones MDP4, MDP7) microsomal dipept; 37872_at Cluster Incl. AF072468:Homo sapiens (JH8) mRNA, partial cds /cds=(0,125; 38239_at Cluster Incl. AI312905:qp84d03.x1 Homo sapiens cDNA, 3 end /clone=IMAG; 39639_s_at Cluster Incl. X07948:Human mRNA for transition protein 1 (TP1) /cds=(; 40006_at Cluster Incl. U63090:Human Gal beta-1,3 GalNAc alpha-2,3 sialyltransfer; 40286_r_at Cluster Incl. AL050370:Homo sapiens mRNA; cDNA DKFZp566C0546 (from cl; 32047_at Cluster Incl. U91985:Human DNA fragmentation factor-45 mRNA, complete c; 33231_at Cluster Incl. AI761148:wh97h07.x1 Homo sapiens cDNA, 3 end /clone=IMAG; 33727_r_at Cluster Incl. AB029011:Homo sapiens mRNA for KIAA1088 protein, partia; 33738_r_at Cluster Incl. AI871359:wl81b11.x1 Homo sapiens cDNA, 3 end /clone=IM; 34287_at Cluster Incl. AB023175:Homo sapiens mRNA for KIAA0958 protein, partial; 40098_at Cluster Incl. AF001434:Human Hpast (HPAST) mRNA, complete cds /cds=(255; 40819_at Cluster Incl. AA161065:zl50h04.r1 Homo sapiens cDNA, 5 end /clone=IMAG; 41218_at Cluster Incl. AB018272:Homo sapiens mRNA for KIAA0729 protein, partial; 33432_at Cluster Incl. AI547308:PN001_AH-B03.r Homo sapiens cDNA, 5 end /clone_{—}; 37297_at Cluster Incl. AL049422:Homo sapiens mRNA; cDNA DKFZp586A191 (from clone; 38371_at Cluster Incl. M64992:Human prosomal protein P30-33K (pros-30) mRNA, com; 39820_at Cluster Incl. AF001549:Human Chromosome 16 BAC clone CIT987SK-A-270G1 /; 41543_at Cluster Incl. U34360:Human lymphoid nuclear protein (LAF-4) mRNA, compl; 382_at X70218 /FEATURE= /DEFINITION=HSPPX Homo sapiens mRNA for protein phosphat.

Metagene 89; 41637_at Cluster Incl. AF108145:Homo sapiens MYLE mRNA, complete cds /cds=(52,25; 35663_at Cluster Incl. U29195:Human neuronal pentraxin II (NPTX2) gene /cds=(58,; 40877_s_at Cluster Incl. AF041080:Homo sapiens D15F37 pseudogene, S3 allele, mRN.

Metagene 90; 36711_at Cluster Incl. AL021977:bK447C4.1 (novel MAFF (v-maf musculoaponeurotic; 38167_at Cluster Incl. AB020704:Homo sapiens mRNA for KIAA0897 protein, partial; 40747_at Cluster Incl. AB002309:Human mRNA for KIAA0311 gene, partial cds /cds=(; 35625_at Cluster Incl. X94630:H. sapiens CD97 gene exon 1 (and joined CDS) /cds=(; 35989_at Cluster Incl. AF007160:Homo sapiens unknown mRNA, partial cds /cds=(0,4; 37193_at Cluster Incl. D78335:Human mRNA for 5-terminal region of UMK, complete; 37576_at Cluster Incl. U52969:Human PEP19 (PCP4) mRNA, complete cds /cds=(81,269; 39044_s_at Cluster Incl. D73409:Homo sapiens mRNA for diacylglycerol kinase delt; 39330_s_at Cluster Incl. M95178:Human non-muscle alpha-actinin mRNA, complete cd; 40855_at Cluster Incl. AB028976:Homo sapiens mRNA for KIAA1053 protein, partial; 32242_at Cluster Incl. AL038340:DKFZp566K192_s1 Homo sapiens cDNA, 3 end /clone; 32243_g_at Cluster Incl. AL038340:DKFZp566K192_s1 Homo sapiens cDNA, 3 end /clo; 33900_at Cluster Incl. U76702:Homo sapiens follistatin-related protein FLRG (FLR; 36175_s_at Cluster Incl. AL023584:Human DNA sequence from clone 67K17 on chromos; 36671_at Cluster Incl. M27396:Human asparagine synthetase mRNA, complete cds /cd; 36956_at Cluster Incl. L20852:Human leukemia virus receptor 2 (GLVR2) mRNA, comp; 39105_at Cluster Incl. Z46389:Homo sapiens encoding vasodilator-stimulated phosp; 32506_at Cluster Incl. AB029031:Homo sapiens mRNA for KIAA1108 protein, partial; 1137_at L20852 /FEATURE= /DEFINITION=HUMGLVR2X Human leukemia virus receptor 2 (; 790_at X52599 /FEATURE=cds /DEFINITION=HSBNGFAC Human mRNA for beta nerve growth; 791_g_at X52599 /FEATURE=cds /DEFINITION=HSBNGFAC Human mRNA for beta nerve grow.

Metagene 91; 33942_s_at Cluster Incl. AF004563:Homo sapiens hUNC18b alternatively-spliced mRN; 38312_at Cluster Incl. AL050002:Homo sapiens mRNA; cDNA DKFZp564O222 (from clone; 39779_at Cluster Incl. U38847:Human TAR RNA loop binding protein (TRP-185) mRNA,; 40434_at Cluster Incl. U97519:Homo sapiens podocalyxin-like protein mRNA, comple; 41189_at Cluster Incl. Y09392:H. sapiens mRNA for WSL-LR, WSL-S1 and WSL-S2 prote; 37375_at Cluster Incl. AB014538:Homo sapiens mRNA for KIAA0638 protein, partial; 38430_at Cluster Incl. AA128249:zl29d09.r1 Homo sapiens cDNA, 5 end /clone=IMAG; 38826_at Cluster Incl. D50918:Human mRNA for KIAA0128 gene, partial cds /cds=(0,; 1954_at AF035121 /FEATURE= /DEFINITION=AF035121 Homo sapiens KDR/flk-1 protein m; 599_at M60721 /FEATURE=mRNA /DEFINITION=HUMHB24 Human homeobox gene, complete cd.

Metagene 92; 31955_at Cluster Incl. X65923:H. sapiens fau mRNA /cds=(56,457) /gb=X65923 /gi=31; 34446_at Cluster Incl. AL049701:Human gene from PAC 433G19, chromosome 1 /cds=(0; 40032_at Cluster Incl. D50923:Human mRNA for KIAA0133 gene, complete cds /cds=(1; 40038_at Cluster Incl. W02490:za48b02.r1 Homo sapiens cDNA, 5 end /clone=IMAGE-; 32042_at Cluster Incl. S72904:APK1 antigen=MAb KI recognized [human, ovarian car; 33242_at Cluster Incl. U92980:Homo sapiens clone DT1P1A10 mRNA, CAG repeat regio; 33315_at Cluster Incl. M29204:Human DNA-binding factor mRNA, complete cds /cds=(; 35724_at Cluster Incl. Y07867:H. sapiens mRNA for Pirin, isolate 1 /cds=(204,1076; 36457_at Cluster Incl. U10860:Human guanosine 5-monophosphate synthase mRNA, com; 36553_at Cluster Incl. AA669799:ag36c04.s1 Homo sapiens cDNA, 3 end /clone=IMAG; 37597_s_at Cluster Incl. AF055006:Homo sapiens clone 24666 sec6 homolog mRNA, pa; 38286_at Cluster Incl. AB028994:Homo sapiens mRNA for KIAA1071 protein, partial; 38648_at Cluster Incl. U80760:Homo sapiens CAGH1 alternate open reading frame mR; 38709_at Cluster Incl. D80009:Human mRNA for KIAA0187 gene, complete cds /cds=(2; 39060_at Cluster Incl. D38048:Human mRNA for proteasome subunit z, complete cds; 39420_at Cluster Incl. S62138:TLS/CHOP=hybrid gene {translocation breakpoint} [h; 40465_at Cluster Incl. AF026402:Homo sapiens U5 snRNP 100 kD protein mRNA, compl; 40515_at Cluster Incl. AF035280:Homo sapiens clone 23689 mRNA, complete cds /cds; 40854_at Cluster Incl. J04973:Human cytochrome bc-1 complex core protein II mRNA; 41122_at Cluster Incl. AB011173:Homo sapiens mRNA for KIAA0601 protein, partial; 41155_at Cluster Incl. U03100:Human alpha2(E)-catenin mRNA, complete cds /cds=(4; 41729_at Cluster Incl. AJ009771:Homo sapiens mRNA for putative RING finger prote; 32213_at Cluster Incl. AA203527:zx56f09.r1 Homo sapiens cDNA, 5 end /clone=IMAG; 32744_at Cluster Incl. AI526078:DU3.2-7.G08.r Homo sapiens cDNA, 5 end /clone e; 32766_at Cluster Incl. Z83840:Human DNA sequence from clone 216E10 on chromosome; 32809_at Cluster Incl. AL118582:DKFZp761B0810_r1 Homo sapiens cDNA, 5 end /clon; 33406_at Cluster Incl. AL050345:Novel human gene mapping to chomosome 22 /cds=(1; 34395_at Cluster Incl. AB002352:Human mRNA for KIAA0354 gene, complete cds /cds=; 35304_at Cluster Incl. AF052130:Homo sapiens clone 23704 mRNA sequence /cds=UNKN; 35355_at Cluster Incl. AB020697:Homo sapiens mRNA for KIAA0890 protein, complete; 35742_at Cluster Incl. U95740:Human Chromosome 16 BAC clone CIT987SK-A-362G6 /cd; 35777_at Cluster Incl. AB000468:Homo sapiens mRNA for zinc finger protein, compl; 37321_at Cluster Incl. U46570:Human tetratricopeptide repeat protein (tpr1) mRNA; 37399_at Cluster Incl. D17793:Human mRNA for KIAA0119 gene, complete cds /cds=(5; 38103_at Cluster Incl. AB014542:Homo sapiens mRNA for KIAA0642 protein, partial; 38400_at Cluster Incl. AI920820:wn82e10.x1 Homo sapiens cDNA, 3 end /clone=IMAG; 38475_at Cluster Incl. U50733:Human dynamitin mRNA, complete cds /cds=(78,1298); 38751_i_at Cluster Incl. AA426364:zv61b06.s1 Homo sapiens cDNA, 3 end /clone=IM; 39152_f_at Cluster Incl. U06632:Homo sapiens p80-coilin mRNA, complete cds /cds=; 39856_at Cluster Incl. AI708983:at02f03.x1 Homo sapiens cDNA, 3 end /clone=IMAG; 40184_at Cluster Incl. L37042:Homo sapiens casein kinase 1 alpha isoform (CSNK1A; 40896_at Cluster Incl. X13403:Human mRNA for octamer-binding protein Oct-1 /cds=; 41268_g_at Cluster Incl. AB028972:Homo sapiens mRNA for KIAA1049 protein, partia; 32511_at Cluster Incl. AI498132:tm90a12.x1 Homo sapiens cDNA, 3 end /clone=IMAG; 688_at L02426 /FEATURE= /DEFINITION=HUM26SPSIV Human 26S protease (S4) regulator.

Metagene 93; 35096_at Cluster Incl. U18244:Human excitatory amino acid transporter 4 mRNA, co; 37521_s_at Cluster Incl. H82458:yv80b07.r1 Homo sapiens cDNA, 5 end /clone=IMAG; 38554_at Cluster Incl. AA903720:ok60c02.s1 Homo sapiens cDNA, 3 end /clone=IMAG; 1063_s_at U02566 /FEATURE= /DEFINITION=HSU02566 Human receptor tyrosine kinase t; 801_at X82676 /FEATURE= /DEFINITION=HSTYRPH Homo sapiens mRNA for tyrosine phosp.

Metagene 94; 39994_at Cluster Incl. D10925:Human mRNA for HM145 /cds=(22,1089) /gb=D10925 /gi; 770_at D00632 /FEATURE= /DEFINITION=HUMGSHPXA Homo sapiens mRNA for glutathione.

Metagene 95; 33708_at Cluster Incl. AF045584:Homo sapiens PB39 mRNA, complete cds /cds=(76,17; 37248_at Cluster Incl. U83411:Homo sapiens carboxypeptidase Z precursor, mRNA, c; 40154_at Cluster Incl. AL096725:Homo sapiens mRNA; cDNA DKFZp434B103 (from clone; 40856_at Cluster Incl. U29953:Human pigment epithelium-derived factor gene, comp; 38124_at Cluster Incl. X55110:Human mRNA for neurite outgrowth-promoting protein; 38406_f_at Cluster Incl. AI207842:ao89h09.x1 Homo sapiens cDNA, 3 end /clone=IM; 38407_r_at Cluster Incl. AI207842:ao89h09.x1 Homo sapiens cDNA, 3 end /clone=IM; 39158_at Cluster Incl. AB021663:Homo sapiens mRNA for leucine-zipper protein, co; 216_at M98539 /FEATURE=exon /DEFINITION=HUMPDS03 Human prostaglandin D2 synthase.

Metagene 96; 37104_at Cluster Incl. L40904:H. sapiens peroxisome proliferator activated recep; 34176_at Cluster Incl. AF091087:Homo sapiens clone 643 unknown mRNA, complete se; 35226_at Cluster Incl. U71207:Human eyes absent homolog (Eab1) mRNA, complete cd; 40255_at Cluster Incl. AC004531:Homo sapiens Chromosome 16 BAC clone CIT987SK-A-; 506_s_at U43185 /FEATURE= /DEFINITION=HSU43185 Human signal transducer and activ.

Metagene 97; 36418_at Cluster Incl. AJ011654:Homo sapiens mRNA for triple LIM domain protein; 38090_at Cluster Incl. AL050371:Homo sapiens mRNA; cDNA DKFZp566G2246 (from clon; 1518_at J04101 /FEATURE= /DEFINITION=HUMETS1A Human erythroblastosis virus oncog.

Metagene 98; 33961_at Cluster Incl. AL109666:Homo sapiens mRNA full length insert cDNA clone; 34456_s_at Cluster Incl. AB007296:Homo sapiens mRNA for hGLI2, complete cds, clo; 35420_r_at Cluster Incl. AB020720:Homo sapiens mRNA for KIAA0913 protein, partia; 38190_r_at Cluster Incl. AB014545:Homo sapiens mRNA for KIAA0645 protein, comple; 41627_at Cluster Incl. D50645:Homo sapiens mRNA for SDF2, complete cds /cds=(39,; 34727_at Cluster Incl. AI800578:wg12b07.x1 Homo sapiens cDNA, 3 end /clone=IMAG; 40152_r_at Cluster Incl. Z48054:H. sapiens mRNA for peroxisomal targeting signal; 32197_at Cluster Incl. AF070548:Homo sapiens clone 24408 2-oxoglutarate carrier; 36964_at Cluster Incl. D42053:Human mRNA for KIAA0091 gene, complete cds /cds=(4; 37004_at Cluster Incl. J02761:Human pulmonary surfactant-associated protein B (S; 37317_at Cluster Incl. L25107:Human LIS mRNA /cds=UNKNOWN /gb=L25107 /gi=602459; 39205_at Cluster Incl. W28793:52f4 Homo sapiens cDNA /gb=W28793 /gi=1308804 /ug=; 39448_r_at Cluster Incl. W27095:20c10 Homo sapiens cDNA /gb=W27095 /gi=1306467 /; 41001_at Cluster Incl. AB023202:Homo sapiens mRNA for KIAA0985 protein, complete; 41280_r_at Cluster Incl. AF007134:Homo sapiens clone 23565 unknown mRNA, partial; 894_g_at M91670 /FEATURE= /DEFINITION=HUME2EPI Human ubiquitin carrier protein (; 293_at Homeotic Protein Hpx-42; 244_at M64673 /FEATURE= /DEFINITION=HUMHSF1 Human heat shock factor 1 (TCF5) mRN.

Metagene 99; 35898_at Cluster Incl. AF100780:Homo sapiens connective tissue growth factor rel; 39971_at Cluster Incl. M22637:Human LYL-1 protein mRNA, complete cds /cds=UNKNOW; 40030_at Cluster Incl. Y15801:Homo sapiens mRNA for PRKY protein /cds=UNKNOWN /g; 32113_at Cluster Incl. U83115:Human non-lens beta gamma-crystallin like protein; 39042_at Cluster Incl. X05615:Human mRNA for thyroglobulin /cds=(41,8344) /gb=X0.

Metagene 100; 38505_at Cluster Incl. AL050151:Homo sapiens mRNA; cDNA DKFZp586J0720 (from clon; 36921_at Cluster Incl. U02556:Human RP3 mRNA, complete cds /cds=(68,418) /gb=U02; 37958_at Cluster Incl. AL049257:Homo sapiens mRNA; cDNA DKFZp564E153 (from clone; 41225_at Cluster Incl. AL049417:Homo sapiens mRNA; cDNA DKFZp586O1919 (from clon; 32210_at Cluster Incl. M83088:Human phosphoglucomutase 1 (PGM1) mRNA, complete c; 32822_at Cluster Incl. J02966:Human mitochondrial ADP/ADT translocator mRNA, com; 32846_s_at Cluster Incl. D13629:Human mRNA for KIAA0004 gene, complete cds /cds=; 33423_g_at Cluster Incl. AF052155:Homo sapiens clone 24761 mRNA sequence /cds=UN; 34370_at Cluster Incl. X81198:H. sapiens mRNA (clone p5) for archain /cds=(78,149; 36585_at Cluster Incl. M36341:Human ADP-ribosylation factor 4 (ARF4) mRNA, compl; 36677_at Cluster Incl. X70476:H. sapiens subunit of coatomer complex /cds=(68,278; 37345_at Cluster Incl. AF013759:Homo sapiens calumein (Calu) mRNA, complete cds; 37366_at Cluster Incl. AL049969:Homo sapiens mRNA; cDNA DKFZp564A072 (from clone; 38113_at Cluster Incl. AB018339:Homo sapiens mRNA for KIAA0796 protein, partial; 40621_at Cluster Incl. U63809:Homo sapiens prostate apoptosis response protein p; 2017_s_at M64349 /FEATURE= /DEFINITION=HUMCYCD1 Human cyclin D (cyclin D1) mRNA,; 1508_at D25303 /FEATURE= /DEFINITION=HUMIAS Human mRNA for integrin alpha subuni; 120_at X68742 /FEATURE= /DEFINITION=HSINTASA H. sapiens mRNA for integrin, alpha.

Metagene 101; 34502_g_at Cluster Incl. L40992:Homo sapiens (clone PEBP2aA1) core-binding facto; 35422_at Cluster Incl. U01828:Human microtubule-associated protein 2 (MAP2) mRNA; 37222_at Cluster Incl. X79389:H. sapiens GSTT1 mRNA /cds=(0,722) /gb=X79389 /gi=5; 1972_s_at U89330 /FEATURE= /DEFINITION=HSU89330 Human alternatively spliced micr; 1035_g_at U14394 /FEATURE= /DEFINITION=HSU14394 Human tissue inhibitor of metall; 363_at Z15114 /FEATURE=cds /DEFINITION=HSPKCG H. sapiens mRNA for protein kinase; 375_at Z84718 /FEATURE=mRNA#1 /DEFINITION=HS322B1 Human DNA sequence from clone.

Metagene 102; 33642_s_at Cluster Incl. U17986:Human GABA/noradrenaline transporter mRNA, compl; 35569_at Cluster Incl. AB015330:Homo sapiens HRIHFB2007 mRNA, partial cds /cds=(; 41117_s_at Cluster Incl. AB016243:Homo sapiens gene for regulatory factor 2 of s; 34179_at Cluster Incl. Z97183:Human DNA sequence from cosmid B2046 on chromosome; 34733_at Cluster Incl. X85237:H. sapiens mRNA for splicing factor SF3a120 /cds=(9; 36564_at Cluster Incl. W27419:31a10 Homo sapiens cDNA /gb=W27419 /gi=1307241 /ug; 37557_at Cluster Incl. U62531:Human AE2 anion exchanger (SLC4A2) mRNA, complete; 38308_g_at Cluster Incl. AB011179:Homo sapiens mRNA for KIAA0607 protein, partia; 38314_at Cluster Incl. AB002304:Human mRNA for KIAA0306 gene, partial cds /cds=(; 38633_at Cluster Incl. U35113:Human metastasis-associated mta1 mRNA, complete cd; 39770_at Cluster Incl. D87437:Human mRNA for KIAA0250 gene, complete cds /cds=(4; 40489_at Cluster Incl. D31840:Human DRPLA mRNA for ORF, complete cds /cds=(238,3; 40850_at Cluster Incl. L37033:Human FK-506 binding protein homologue (FKBP38) nmR; 37370_i_at Cluster Incl. L41066:Homo sapiens NF-AT3 mRNA, complete cds /cds=(141; 37386_i_at Cluster Incl. X55885:Human mRNA for a presumptive KDEL receptor /cds=; 37387_r_at Cluster Incl. X55885:Human mRNA for a presumptive KDEL receptor /cds=; 37652_at Cluster Incl. AB002328:Human mRNA for KIAA0330 gene, partial cds /cds=(; 38775_at Cluster Incl. X13916:Human mRNA for LDL-receptor related protein /cds=(; 38828_s_at Cluster Incl. AA628946:af28f05.s1 Homo sapiens cDNA, 3 end /clone=IM; 39530_at Cluster Incl. L35240:Human enigma gene, complete cds /cds=(0,1367) /gb=; 41337_at Cluster Incl. AF072902:Homo sapiens gp130 associated protein GAM mRNA,; 1643_g_at U35113 /FEATURE= /DEFINITION=HSU35113 Human metastasis-associated mta1; 1011_s_at U54778 /FEATURE= /DEFINITION=HSU54778 Human 14-3-3 epsilon mRNA, compl; 880_at M34539 /FEATURE= /DEFINITION=HUMFKBP Human FK506-binding protein (FKBP) m; 657_at L11373 /FEATURE= /DEFINITION=HUMPC43ABB Human protocadherin 43 mRNA, comp; 249_at L41066 /FEATURE= /DEFINITION=HUMNFAT3A Homo sapiens NF-AT3 mRNA, complete.

Metagene 103; 39294_at Cluster Incl. X16155:Human mRNA for chicken ovalbumin upstream promoter; 33785_at Cluster Incl. AB005298:Homo sapiens BAI 2 mRNA, complete cds /cds=(378,; 32208_at Cluster Incl. AB002353:Human mRNA for KIAA0355 gene, complete cds /cds=.

Metagene 104; 33648_at Cluster Incl. W28800:52g12 Homo sapiens cDNA /gb=W28800 /gi=1308811 /ug; 34082_at Cluster Incl. W28356:48e3 Homo sapiens cDNA /gb=W28356 /gi=1308511 /ug=; 35520_at Cluster Incl. AI701514:we35h02.x1 Homo sapiens cDNA, 3 end /clone=IMAG; 40044_at Cluster Incl. U16282:Human ELL mRNA, complete cds /cds=(12,1877) /gb=U1; 41726_at Cluster Incl. Z35307:H. sapiens mRNA for endothelin-converting-enzyme 1; 32185_at Cluster Incl. U00946:Human clone A9A2BRB5 (CAC)n/(GTG)n repeat-containi; 41501_at Cluster Incl. AF004849:Homo sapiens PKY protein kinase mRNA, complete c; 517_at U07000 /FEATURE=cds#4 /DEFINITION=HSU07000 Human breakpoint cluster regio.

Metagene 105; 31740_s_at Cluster Incl. AB008913:Homo sapiens mRNA for Pax-4, complete cds /cds; 40649_at Cluster Incl. X64810:H. sapiens encoding PC1/PC3 /cds=(189,2450) /gb=X64; 40723_at Cluster Incl. AJ010059:Homo sapiens SIT protein /cds=(87,677) /gb=AJ010; 33322_i_at Cluster Incl. X57348:H. sapiens mRNA (clone 9112) /cds=(165,911) /gb=X; 36030_at Cluster Incl. AL080214:Homo sapiens mRNA; cDNA DKFZp586I2223 (from clon; 36079_at Cluster Incl. AF010309:Homo sapiens Pig3 (PIG3) mRNA, complete cds /cds; 40117_at Cluster Incl. D84557:Homo sapiens mRNA for HsMcm6, complete cds /cds=(6; 41171_at Cluster Incl. D45248:Human mRNA for proteasome activator hPA28 subunit; 35312_at Cluster Incl. D21063:Human mRNA for KIAA0030 gene, partial cds /cds=(0,; 36170_at Cluster Incl. D83198:Homo sapiens mRNA expressed in thyroid gland /cds=; 37323_r_at Cluster Incl. X82460:H. sapiens mRNA for 15-hydroxy prostaglandin dehy; 38788_at Cluster Incl. M82827:Human fusion protein mRNA, complete cds /cds=(324,; 1184_at D45248 /FEATURE= /DEFINITION=HUMPHPA28A Human mRNA for proteasome activa; 709_at J00314 /FEATURE=mRNA#1 /DEFINITION=HUMTBBM40 Human beta-tubulin gene, clo.

Metagene 106; 37492_at Cluster Incl. AB007969:Homo sapiens mRNA, chromosome 1 specific transcr; 37277_at Cluster Incl. U80017:Homo sapiens basic transcription factor 2 p44 (btf; 39037_at Cluster Incl. L13773:Human AF-4 mRNA, complete cds /cds=(420,4052) /gb=; 39777_at Cluster Incl. AF075587:Homo sapiens protein associated with Myc mRNA, c; 40067_at Cluster Incl. M82882:Human cis-acting sequence /cds=UNKNOWN /gb=M82882; 38065_at Cluster Incl. X62534:H. sapiens HMG-2 mRNA /cds=(214,843) /gb=X62534 /gi.

Metagene 107; 35605_at Cluster Incl. AL049653:dJ647M16.1 (Angiopoietin-like factor); likely to; 34957_at Cluster Incl. Y18504:Homo sapiens X5L gene /cds=(112,1089) /gb=Y18504 /; 36739_at Cluster Incl. U54617:Human pyruvate dehydrogenase kinase isoform 4 mRNA; 39681_at Cluster Incl. AF060568:Homo sapiens promyelocytic leukemia zinc finger; 41032_at Cluster Incl. U56387:Human PC6A protease (hPC6) mRNA, complete cds /cds; 33326_at Cluster Incl. D42087:Human mRNA for KIAA0118 gene, partial cds /cds=(0,; 33783_at Cluster Incl. AB007867:Homo sapiens KIAA0407 mRNA, complete cds /cds=(2; 36034_at Cluster Incl. D87447:Human mRNA for KIAA0258 gene, complete cds /cds=(8; 36917_at Cluster Incl. Z26653:H. sapiens mRNA for laminin M chain (merosin) /cds=; 37230_at Cluster Incl. AB007938:Homo sapiens mRNA for KIAA0469 protein, complete; 38317_at Cluster Incl. M99701:Homo sapiens (pp21) mRNA, complete cds /cds=(164,6; 38340_at Cluster Incl. AB014555:Homo sapiens mRNA for KIAA0655 protein, partial; 38999_s_at Cluster Incl. M86707:Homo sapiens myristoyl CoA-protein N-myristoyltr; 40162_s_at Cluster Incl. AC003107:Human DNA from chromosome 19-specific cosmid R; 40861_at Cluster Incl. D14812:Human mRNA for KIAA0026 gene, complete cds /cds=(3; 34377_at Cluster Incl. J05096:Human Na,K-ATPase subunit alpha 2 (ATP1A2) gene, c; 35782_at Cluster Incl. AB014557:Homo sapiens mRNA for KIAA0657 protein, partial; 37367_at Cluster Incl. X76228:H. sapiens mRNA for vacuolar H+ ATPase E subunit /c; 37748_at Cluster Incl. D86985:Human mRNA for KIAA0232 gene, complete cds /cds=(5; 38734_at Cluster Incl. M63603:Human phospholamban mRNA, complete cds /cds=(181,3; 39098_at Cluster Incl. X52896:H. sapiens RNA for dermal fibroblast elastin /cds=U; 40971_at Cluster Incl. D86982:Human mRNA for KIAA0229 gene, partial cds /cds=(0,; 40973_at Cluster Incl. AI146846:qb92h04.x1 Homo sapiens cDNA, 3 end /clone=IMAG; 33136_at Cluster Incl. AL031714:Human DNA sequence from clone 356B7 on chromosom; 1911_s_at M60974 /FEATURE= /DEFINITION=HUMGADD45 Human growth arrest and DNA-dam.

Metagene 108; 31641_s_at Cluster Incl. AL031282:dJ283E3.2.4 (Matrix Metalloproteinase MMP21/22; 40709_at Cluster Incl. W27601:35a3 Homo sapiens cDNA /gb=W27601 /gi=1307549 /ug=; 34218_at Cluster Incl. U57099:Human APEG-1 mRNA, complete cds /cds=(125,466) /gb; 35137_at Cluster Incl. X69090:H. sapiens mRNA for skeletal muscle 190 kD protein /; 36039_s_at Cluster Incl. X93498:H. sapiens mRNA for 21-Glutamic Acid-Rich Protein; 38248_at Cluster Incl. AB011124:Homo sapiens mRNA for KIAA0552 protein, complete; 39423_f_at Cluster Incl. AJ000644:Homo sapiens mRNA for SPOP /cds=(157,1281) /gb; 39717_g_at Cluster Incl. AI597616:tn15f08.x1 Homo sapiens cDNA, 3 end /clone=IM; 41207_at Cluster Incl. AF043897:Homo sapiens C90RF3 large isoform, mRNA sequence; 33885_at Cluster Incl. AB020714:Homo sapiens mRNA for KIAA0907 protein, complete; 34870_at Cluster Incl. AB014513:Homo sapiens mRNA for KIAA0613 protein, partial; 36142_at Cluster Incl. X79204:H. sapiens SCA1 mRNA for ataxin /cds=(935,3385) /gb; 36144_at Cluster Incl. D38522:Human mRNA for KIAA0080 gene, partial cds /cds=(0,; 36577_at Cluster Incl. Z24725:H. sapiens mitogen inducible gene mig-2, complete C; 37304_at Cluster Incl. U35451:Homo sapiens heterochromatin protein p25 mRNA, com; 38101_at Cluster Incl. AB011151:Homo sapiens mRNA for KIAA0579 protein, partial; 38396_at Cluster Incl. Y09836:H. sapiens mRNA for 3UTR of unknown protein /cds=UN; 40575_at Cluster Incl. AB011155:Homo sapiens mRNA for KIAA0583 protein, partial; 40994_at Cluster Incl. L15388:Human G protein-coupled receptor kinase (GRK5) mRN; 41273_at Cluster Incl. AL046940:DKFZp586I0517_r1 Homo sapiens cDNA, 5 end /clon; 32554_s_at Cluster Incl. Y12781:Homo sapiens mRNA for transducin (beta) like 1 p; 2056_at M34641 /FEATURE= /DEFINITION=HUMFGF1A Human fibroblast growth factor (FG; 2057_g_at M34641 /FEATURE= /DEFINITION=HUMFGF1A Human fibroblast growth factor (; 1535_at U68723 /FEATURE= /DEFINITION=HSU68723 Human checkpoint suppressor 1 mRNA; 1230_g_at U78556 /FEATURE= /DEFINITION=HSU78556 Human cisplatin resistance assoc; 1135_at L15388 /FEATURE= /DEFINITION=HUMGRK5A Human G protein-coupled receptor k; 822_s_at U79115 /FEATURE= /DEFINITION=HSU79115 Human death adaptor molecule RAID; 738_at D38524 /FEATURE= /DEFINITION=HUMSN Human mRNA for 5-nucleotidase; 498_at U33821 /FEATURE= /DEFINITION=HSU33821 Homo sapiens tax1-binding protein T.

Metagene 109; 40022_at Cluster Incl. AB008226:Homo sapiens FCMD mRNA for fukutin, complete cds; 41099_at Cluster Incl. X84740:H. sapiens mRNA for DNA ligase III /cds=(333,3101); 41696_at Cluster Incl. AI620381:tu94d05.x1 Homo sapiens cDNA, 3 end /clone=IMAG; 33331_at Cluster Incl. U17077:Human BENE mRNA, partial cds /cds=(0,446) /gb=U170; 35216_at Cluster Incl. X79440:H. sapiens mRNA for NADP+-dependent malic enzyme /c; 35657_at Cluster Incl. U08998:Human TAR RNA binding protein 2 (TRBP2) mRNA, comp; 36461_at Cluster Incl. U41804:Human putative T1/ST2 receptor binding protein pre; 37252_at Cluster Incl. U44755:Human PSE-binding factor PTF delta subunit mRNA, c; 39745_at Cluster Incl. AB011139:Homo sapiens mRNA for KIAA0567 protein, partial; 40106_at Cluster Incl. AJ007509:Homo sapiens mRNA for E1B-55 kDa-associated prote; 34862_at Cluster Incl. AA005018:zh96a09.r1 Homo sapiens cDNA, 5 end /clone=IMAG; 513_at U25265 /FEATURE= /DEFINITION=HSU25265 Human MEK5 mRNA, complete cds; 187_at U07349 /FEATURE= /DEFINITION=HSU07349 Human B lymphocyte serine/threonine.

Metagene 110; 39253_s_at Cluster Incl. M29893:Human low molecular mass GTP-binding protein (ra; 39323_at Cluster Incl. U45974:Human phosphatidylinositol (4,5) bisphosphate 5-ph; 32057_at Cluster Incl. U32907:Human p37NB mRNA, complete cds /cds=(281,1222) /gb; 33228_g_at Cluster Incl. AI984234:wz57e04.x1 Homo sapiens cDNA, 3 end /clone=IM; 33328_at Cluster Incl. W28612:49b3 Homo sapiens cDNA /gb=W28612 /gi=1308560 /ug=; 41179_at Cluster Incl. AB029023:Homo sapiens mRNA for KIAA1100 protein, complete; 34363_at Cluster Incl. Z11793:H. sapiens mRNA for selenoprotein P /cds=(36,1181); 37303_at Cluster Incl. AF057160:Homo sapiens putative poly(ADP-ribosyl) transfer; 37661_at Cluster Incl. J04027:Human plasma membrane Ca2+ pumping ATPase mRNA, co; 37671_at Cluster Incl. S78569:laminin alpha 4 chain [human, fetal lung, mRNA, 62; 41274_at Cluster Incl. AA908993:ol10d03.s1 Homo sapiens cDNA, 3 end /clone=IMAG; 41504_s_at Cluster Incl. AF055376:Homo sapiens short form transcription factor C; 33126_at Cluster Incl. L13435:Human chromosome 3p21.1 gene sequence /cds=UNKNOWN; 1715_at U37518 /FEATURE= /DEFINITION=HSU37518 Human TNF-related apoptosis induci; 1380_at M60828 /FEATURE= /DEFINITION=HUMKGF Human keratinocyte growth factor mRN.

Metagene 111; 37161_at Cluster Incl. W28948:54b12 Homo sapiens cDNA /gb-W28948 /gi=1308896 /ug; 39597_at Cluster Incl. AB020650:Homo sapiens mRNA for KIAA0843 protein, complete; 41370_at Cluster Incl. AF090988:Homo sapiens U5 snRNP-specific 40 kDa protein mR; 41399_at Cluster Incl. AB029034:Homo sapiens mRNA for KIAA1111 protein, partial; 41712_at Cluster Incl. AB011132:Homo sapiens mRNA for KIAA0560 protein, complete; 31786_at Cluster Incl. AF051321:Homo sapiens Sam68-like phosphotyrosine protein; 36537_at Cluster Incl. AB011093:Homo sapiens mRNA for KIAA0521 protein, partial; 36858_at Cluster Incl. D25218:Human mRNA for KIAA0112 gene, partial cds /cds=(0,; 38702_at Cluster Incl. AF070640:Homo sapiens clone 24781 mRNA sequence /cds=UNKN; 39386_at Cluster Incl. D14811:Human mRNA for KIAA0110 gene, complete cds /cds=(3; 39687_at Cluster Incl. AI524873:promrna-10.C03.r Homo sapiens cDNA, 5 end /clon; 40774_at Cluster Incl. X74801:H. sapiens Cctg mRNA for chaperonin /cds=(0,1634) /; 41756_at Cluster Incl. AJ010842:Homo sapiens mRNA for putative ATP(GTP)-binding; 32237_at Cluster Incl. D87454:Human mRNA for KIAA0265 gene, partial cds /cds=(0,; 35313_at Cluster Incl. AB002308:Human mRNA for KIAA0310 gene, complete cds /cds=; 35750_at Cluster Incl. AL049948:Homo sapiens mRNA; cDNA DKFZp564K0222 (from clon; 36957_at Cluster Incl. W22296:65A11 Homo sapiens cDNA /clone=(not-directional) /; 37726_at Cluster Incl. X06323:Human MRL3 mRNA for ribosomal protein L3 homologue; 40189_at Cluster Incl. M93651:Human set gene, complete cds /cds=(3,836) /gb=M936; 40607_at Cluster Incl. U97105:Homo sapiens N2A3 mRNA, complete cds /cds=(1336,30; 40623_at Cluster Incl. AI749193:at40e04.x1 Homo sapiens cDNA, 3 end /clone=IMAG; 40992_s_at Cluster Incl. AF055993:Homo sapiens mSin3A associated polypeptide p30; 41360_at Cluster Incl. AA044787:zk74c11.r1 Homo sapiens cDNA, 5 end /clone=IMAG; 1195_s_at AF012024 /FEATURE= /DEFINITION=AF012024 Homo sapiens integrin cytoplas.

Metagene 112; 31575_f_at Cluster Incl. M14087:Human HL14 gene encoding beta-galactoside-bindin; 32396_f_at Cluster Incl. AA079018:zm94e12.s1 Homo sapiens cDNA, 3 end /clone=IM; 33633_at Cluster Incl. AF030335:Homo sapiens purinergic P2Y11 receptor (P2Y11) m; 34071_at Cluster Incl. Y11918:H. sapiens IMAGE cDNA clone 26881 /cds=UNKNOWN /gb=; 37098_at Cluster Incl. D38537:Human mRNA for protoporphyrinogen oxidase, complet; 41116_at Cluster Incl. AI799802:wc43d09.x1 Homo sapiens cDNA, 3 end /clone=IMAG; 41414_at Cluster Incl. AL050346:Novel human gene mapping to chomosome 22 /cds=(3; 33269_at Cluster Incl. AB003723:Homo sapiens mRNA for GPI1, complete cds /cds=(1; 35628_at Cluster Incl. AF023676:Homo sapiens lamin B receptor homolog TM7SF2 (TM; 38425_at Cluster Incl. U49719:Human hydroxymethylglutaryl-CoA lyase (HMGCL) gene; 40234_at Cluster Incl. X96484:H. sapiens mRNA for DGCR6 protein /cds=(422,676) /g; 991_at X51602 /FEATURE=cds /DEFINITION=HSFLT Human flt mRNA for receptor-relat.

Metagene 113; 34539_at Cluster Incl. AF065854:Homo sapiens OR7E12P pseudogene, complete sequen; 35585_at Cluster Incl. X15675:Human pTR7 mRNA for repetitive sequence /cds=UNKNO; 37444_at Cluster Incl. AF028827:Homo sapiens Tax interaction protein 40 mRNA, pa; 38518_at Cluster Incl. Y18004:Homo sapiens mRNA for SCML2 protein /cds=(91,2193); 41648_at Cluster Incl. X78706:H. sapiens mRNA for carnitine acetyltransferase /cd; 35983_at Cluster Incl. AC004528:Homo sapiens chromosome 19, cosmid R32184 /cds=(; 39328_at Cluster Incl. M11058:Human 3-hydroxy-3-methylglutaryl coenzyme A reduct; 40054_at Cluster Incl. D43949:Human mRNA for KIAA0082 gene, partial cds /cds=(0,; 40783_s_at Cluster Incl. L36151:Homo sapiens phosphatidylinositol 4-kinase mRNA,; 41749_at Cluster Incl. U53003:Human GT335 mRNA, complete cds /cds=(84,890) /gb=U; 41773_at Cluster Incl. U58048:Human metallopeptidase PRSM1 mRNA, complete cds /c; 34406_at Cluster Incl. AB011174:Homo sapiens mRNA for KIAA0602 protein, partial; 833_at U40279 /FEATURE=cds /DEFINITION=HSITGAD06 Human beta-2 integrin alphaD su.

Metagene 114; 33484_at Cluster Incl. Y10571:H. sapiens mRNA for dinG gene /cds=(12,1022) /gb=Y1; 34472_at Cluster Incl. AB012911:Homo sapiens mRNA for Frizzled-6, complete cds /; 36692_at Cluster Incl. AF052099:Homo sapiens clone 23632 mRNA sequence /cds=UNKN; 38573_at Cluster Incl. U72209:Human YY1-associated factor 2 (YAF2) mRNA, complet; 38605_at Cluster Incl. AI345944:qp47e09.x1 Homo sapiens cDNA, 3 end /clone=IMAG; 40347_at Cluster Incl. AA913812:ol39a08.s1 Homo sapiens cDNA, 3 end /clone=IMAG; 41379_at Cluster Incl. AB011166:Homo sapiens mRNA for KIAA0594 protein, partial; 41381_at Cluster Incl. AB002306:Human mRNA for KIAA0308 gene, partial cds /cds=(; 41441_at Cluster Incl. M55654:Human TATA-binding protein mRNA, complete cds /cds; 31872_at Cluster Incl. X79201:H. sapiens mRNA for SYT /cds=(3,1178) /gb=X79201 /g; 31883_at Cluster Incl. AF025794:Homo sapiens methionine synthase reductase (MTRR; 32621_at Cluster Incl. M97388:Human TATA binding protein-associated phosphoprote; 32669_at Cluster Incl. AB014571:Homo sapiens mRNA for KIAA0671 protein, complete; 32706_at Cluster Incl. X89887:Homo sapiens mRNA for WD repeat protein (HIRA) /cd; 32720_at Cluster Incl. AA151716:zo30d07.s1 Homo sapiens cDNA, 3 end /clone=IMAG; 32735_at Cluster Incl. AB023148:Homo sapiens mRNA for KIAA0931 protein, partial; 33219_at Cluster Incl. AB029020:Homo sapiens mRNA for KIAA1097 protein, partial; 34699_at Cluster Incl. AL050105:Homo sapiens mRNA; cDNA DKFZp586H0519 (from clon; 34728_g_at Cluster Incl. AI800578:wg12b07.x1 Homo sapiens cDNA, 3 end /clone=IM; 35231_at Cluster Incl. X12791:Human mRNA for 19 kD protein of signal recognition; 35656_at Cluster Incl. AJ010346:Homo sapiens mRNA for RING-H2 protein RNF6, alte; 36033_at Cluster Incl. AL049309:Homo sapiens mRNA; cDNA DKFZp564B176 (from clone; 36508_at Cluster Incl. AF030186:Homo sapiens glypican-4 (GPC4) mRNA, complete cd; 36511_at Cluster Incl. AB020658:Homo sapiens mRNA for KIAA0851 protein, complete; 37575_at Cluster Incl. AL050192:Homo sapiens mRNA; cDNA DKFZp586C1723 (from clon; 37900_at Cluster Incl. AF093670:Homo sapiens peroxisomal biogenesis factor (PEX1; 38283_at Cluster Incl. AB007619:Homo sapiens mRNA for EBAG9, complete cds /cds=(; 38668_at Cluster Incl. AB011125:Homo sapiens mRNA for KIAA0553 protein, partial; 38705_at Cluster Incl. AI310002:qo77c11.x1 Homo sapiens cDNA, 3 end /clone=IMAG; 38708_at Cluster Incl. AF054183:Homo sapiens GTP binding protein mRNA, complete; 38967_at Cluster Incl. AF054175:Homo sapiens mitochondrial proteolipid 68MP homo; 38990_at Cluster Incl. AL031178:Human DNA sequence from clone 341E18 on chromoso; 39034_at Cluster Incl. AL080122:Homo sapiens mRNA; cDNA DKFZp564O123 (from clone; 39382_at Cluster Incl. AB011089:Homo sapiens mRNA for KIAA0517 protein, partial; 39442_at Cluster Incl. AL080115:Homo sapiens mRNA; cDNA DKFZp564G0222 (from clon; 39691_at Cluster Incl. AB007960:chromosome 1 specific transcript KIAA0491 /cds=U; 39713_at Cluster Incl. AJ132440:Homo sapiens mRNA for PLU-1 protein /cds=(89,472; 39759_at Cluster Incl. AL031781:dJ51J12.1 (human ortholog of zebrafish Quaking p; 40086_at Cluster Incl. D87450:Human mRNA for KIAA0261 gene, partial cds /cds=(0,; 40128_at Cluster Incl. D79993:Human mRNA for KIAA0171 gene, complete cds /cds=(1; 40457_at Cluster Incl. AF038250:AF038250 Homo sapiens cDNA /clone=ntcon9 /gb=AF0; 40517_at Cluster Incl. AB002370:Human mRNA for KIAA0372 gene, complete cds /cds=; 40831_at Cluster Incl. AL050190:Homo sapiens mRNA; cDNA DKFZp586B0923 (from clon; 41131_f_at Cluster Incl. U01923:Human BTK region clone ftp-3 mRNA /cds=UNKNOWN /; 32769_at Cluster Incl. AB023210:Homo sapiens mRNA for KIAA0993 protein, partial; 32849_at Cluster Incl. D80000:Human mRNA for KIAA0178 gene, partial cds /cds=(0,; 33367_s_at Cluster Incl. D88674:Homo sapiens mRNA for antizyme inhibitor, comple; 33394_at Cluster Incl. AA034074:zi06c05.r1 Homo sapiens cDNA, 5 end /clone=IMAG; 33403_at Cluster Incl. AL050260:Homo sapiens mRNA; cDNA DKFZp547E1010 (from clon; 33877_s_at Cluster Incl. AB028990:Homo sapiens mRNA for KIAA1067 protein, partia; 33886_at Cluster Incl. AF006516:Homo sapiens eps8 binding protein e3B1 mRNA, com; 34349_at Cluster Incl. AJ011779:Homo sapiens mRNA for SEC63 protein /cds=(98,238; 34781_at Cluster Incl. D84145:Human WS-3 mRNA, complete cds /cds=(87,659) /gb=D8; 34819_at Cluster Incl. D14043:Human mRNA for MGC-24, complete cds /cds=(79,648); 34824_at Cluster Incl. AB015344:Homo sapiens HRIHFB2157 mRNA, partial cds /cds=(; 35268_at Cluster Incl. AL050171:Homo sapiens mRNA; cDNA DKFZp586F1122 (from clon; 35271_at Cluster Incl. AF006083:Homo sapiens actin-related protein Arp3 (ARP3) m; 35290_at Cluster Incl. AL050081:Homo sapiens mRNA; cDNA DKFZp566J2146 (from clon; 35306_at Cluster Incl. AB001636:Homo sapiens mRNA for ATP-dependent RNA helicase; 35734_at Cluster Incl. AI935551:wo97g09.x1 Homo sapiens cDNA, 3 end /clone=IMAG; 35761_at Cluster Incl. AL050073:Homo sapiens mRNA; cDNA DKFZp566E2346 (from clon; 35793_at Cluster Incl. AB014560:Homo sapiens mRNA for KIAA0660 protein, complete; 35818_at Cluster Incl. D00265:Homo sapiens mRNA for cytochrome c, partial cds /c; 36110_at Cluster Incl. M28215:Homo sapiens GTP-binding protein (RAB5) mRNA, comp; 36176_at Cluster Incl. U61234:Human tubulin-folding cofactor C mRNA, complete cd; 36579_at Cluster Incl. D50916:Human mRNA for KIAA0126 gene, complete cds /cds=(7; 36604_at Cluster Incl. D83004:Human epidermoid carcinoma mRNA for ubiquitin-conj; 36608_at Cluster Incl. D55654:Human mRNA for cytosolic malate dehydrogenase, com; 36655_at Cluster Incl. L27476:Human X104 mRNA, complete cds /cds=(79,3429) /gb=L; 36943_r_at Cluster Incl. U81992:Homo sapiens C2H2 zinc finger protein PLAGL1 (PL; 37036_at Cluster Incl. AB002299:Human mRNA for KIAA0301 gene, partial cds /cds=(; 37296_at Cluster Incl. L28997:Homo sapiens ARL1 mRNA, complete cds /cds=(144,689; 37336_at Cluster Incl. D87684:Human mRNA for KIAA0242 gene, partial cds /cds=(0,; 37359_at Cluster Incl. D14658:Human mRNA for KIAA0102 gene, complete cds /cds=(3; 37392_at Cluster Incl. X84908:H. sapiens mRNA for phosphorylase-kinase, beta subu; 37663_at Cluster Incl. X70649:Homo sapiens DDX1 gene, complete CDS /cds=(288,251; 37670_at Cluster Incl. J04543:Human synexin mRNA, complete cds /cds=(60,1460) /g; 38016_at Cluster Incl. M94630:Homo sapiens hnRNP-C like protein mRNA, complete c; 38035_at Cluster Incl. AF072928:Homo sapiens myotubularin related protein 6 mRNA; 38040_at Cluster Incl. AF107463:Homo sapiens splicing factor mRNA, complete cds; 38074_at Cluster Incl. U91932:Homo sapiens AP-3 complex sigma3A subunit mRNA, co; 38102_at Cluster Incl. W28575:51f12 Homo sapiens cDNA /gb=W28575 /gi=1308730 /ug; 38441_s_at Cluster Incl. X59408:H. sapiens, gene for Membrane cofactor protein /c; 38462_at Cluster Incl. U64028:Human NADH-ubiquinone oxidoreductase subunit B13 m; 38472_at Cluster Incl. D63477:Human mRNA for KIAA0143 gene, partial cds /cds=(0,; 38835_at Cluster Incl. U94831:Homo sapiens multispanning membrane protein mRNA,; 38846_at Cluster Incl. AA768912:nz82h06.s1 Homo sapiens cDNA /clone=IMAGE-130201; 39099_at Cluster Incl. X97064:H. sapiens mRNA for Sec23A isoform, 2748 bp /cds=(15; 39117_at Cluster Incl. AB014562:Homo sapiens mRNA for KIAA0662 protein, partial; 40262_at Cluster Incl. AF031166:Homo sapiens SRp46 splicing factor retropseudoge; 40901_at Cluster Incl. U17989:Homo sapiens nuclear autoantigen GS2NA mRNA, compl; 41320_s_at Cluster Incl. U69609:Human transcriptional repressor (GCF2) mRNA, com; 41342_at Cluster Incl. D38076:Human mRNA for RanBP1 (Ran-binding protein 1), com; 41562_at Cluster Incl. L13689:Human prot-oncogene (BMI-1) mRNA, complete cds /cd; 41823_at Cluster Incl. AJ132258:Homo sapiens mRNA for staufen protein, partial /; 32508_at Cluster Incl. AL096857:Novel human mRNA from chromosome 1, which has si; 33107_at Cluster Incl. AB020705:Homo sapiens mRNA for KIAA0898 protein, partial; 1789_at U65928 /FEATURE= /DEFINITION=HSU65928 Human Jun activation domain bindin; 1329_s_at U74382 /FEATURE= /DEFINITION=HSU74382 Human telomeric repeat DNA-bindi; 1238_at U09759 /FEATURE= /DEFINITION=HSU09759 Human protein kinase (JNK2) mRNA,; 1046_at U34605 /FEATURE= /DEFINITION=HSU34605 Human retinoic acid- and interfero; 891_at M77698 /FEATURE= /DEFINITION=HUMKRP Homo sapiens GLI-Krupple related prot; 812_at U68111 /FEATURE=mRNA /DEFINITION=HSPPP1R2E6 Human protein phosphatase inh; 630_at L39874 /FEATURE=expanded_cds /DEFINITION=HUMDODDA Homo sapiens deoxycytid; 509_at U44378 /FEATURE= /DEFINITION=HSU44378 Human homozygous deletion target in; 510_g_at U44378 /FEATURE= /DEFINITION=HSU44378 Human homozygous deletion target; 488_at U61166 /FEATURE= /DEFINITION=HSU61166 Human SH3 domain-containing protein; 379_at AB006679 /FEATURE= /DEFINITION=AB006679 Homo sapiens mRNA for ATP binding.

Metagene 115; 34099_f_at Cluster Incl. W26056:18e1 Homo sapiens cDNA /gb=W26056 /gi=1306323 /u; 35408_i_at Cluster Incl. X16281:Human mRNA for zinc finger protein (clone 431) /; 39360_at Cluster Incl. AF034546:Homo sapiens sorting nexin 3 (SNX3) mRNA, comple; 32232_at Cluster Incl. AF047181:Homo sapiens NADH-ubiquinone oxidoreductase subu; 39826_f_at Cluster Incl. W29115:56e8 Homo sapiens cDNA /gb=W29115 /gi=1309081 /u; 1668_s_at L15409 /FEATURE= /DEFINITION=HUMHIPLIND Homo sapiens (clone g7) von Hi.

Metagene 116; 32021_at Cluster Incl. AI560890:tq41d05.x1 Homo sapiens cDNA, 3 end /clone=IMAG; 33035_at Cluster Incl. AL021397:dJ69E111.3 (Yeast YPR037W and worm C02C2.6 predic; 35413_s_at Cluster Incl. AA258092:zs30g01.r1 Homo sapiens cDNA, 5 end /clone=IM; 35870_at Cluster Incl. AJ007714:Homo sapiens mRNA for lysine-ketoglutarate reduc; 35963_at Cluster Incl. AI201243:qf70f09.x1 Homo sapiens cDNA, 3 end /clone=IMAG; 37506_at Cluster Incl. Z78308:HSZ78308 Homo sapiens cDNA /clone=1.47-(CEPH) /gb=; 37507_i_at Cluster Incl. AA675900:g02504r Homo sapiens cDNA, 5 end /clone=g0250; 40713_at Cluster Incl. AB020634:Homo sapiens mRNA for KIAA0827 protein, complete; 32124_at Cluster Incl. AL030996:dJ1189B24.4 (novel PUTATIVE protein similar to h; 39436_at Cluster Incl. AF079221:Homo sapiens BCL2 /adenovirus E1B 19 kDa-interacti; 38807_at Cluster Incl. AA043348:zk66b12.s1 Homo sapiens cDNA, 3 end /clone=IMAG; 38814_at Cluster Incl. AF038954:Homo sapiens vacuolar H(+)-ATPase subunit mRNA,; 40920_at Cluster Incl. AF023158:Homo sapiens tyrosine phosphatase (cdc14B) mRNA,; 450_g_at U66469 /FEATURE= /DEFINITION=HSU66469 Human cell growth regulator CGR19; 147_at U82130 /FEATURE= /DEFINITION=HSU82130 Human tumor susceptiblity protein (.

Metagene 117; 32030_at Cluster Incl. X99459:H. sapiens mRNA for sigma 3B protein /cds=(30,611); 35984_at Cluster Incl. AF041381:Homo sapiens putative transcriptional repressor; 40827_at Cluster Incl. U04953:Human isoleucyl-tRNA synthetase mRNA, complete cds; 41750_at Cluster Incl. D49489:Human mRNA for protein disulfide isomerase-related; 33369_at Cluster Incl. AI535653:P9-C4.T3.P9.D4 Homo sapiens cDNA, 3 end /clone_{—}; 33422_at Cluster Incl. AF052155:Homo sapiens clone 24761 mRNA sequence /cds=UNKN; 34390_at Cluster Incl. U90441:Human prolyl 4-hydroxylase alpha (II) subunit mRNA; 35340_at Cluster Incl. AI819948:wj88e11.x1 Homo sapiens cDNA, 3 end /clone=IMAG; 36992_at Cluster Incl. AI653621:tz21b11.x1 Homo sapiens cDNA, 3 end /clone=IMAG; 37046_at Cluster Incl. AI246726:qk40a09.x1 Homo sapiens cDNA, 3 end /clone=IMAG; 40584_at Cluster Incl. Y08612:Homo sapiens mRNA for nuclear pore complex protein; 32539_at Cluster Incl. U51205:Human COP9 homolog (HCOP9) mRNA, complete cds /cds; 890_at M74524 /FEATURE= /DEFINITION=HHUMHHR6A Human HHR6A (yeast RAD 6 homologue).

Metagene 118; 31481_s_at Cluster Incl. M92383:Homo sapiens thymosin beta-10 gene, 3end /cds=(0; 32434_at Cluster Incl. D10522:Homo sapiens mRNA for 80K-L protein, complete cds; 32321_at Cluster Incl. X56841:H. sapiens HLA-E gene /cds=(0,363) /gb=X56841 /gi=4; 35012_at Cluster Incl. M81750:H. sapiens myeloid cell nuclear differentiation ant; 37509_at Cluster Incl. AF046059:Homo sapiens cytokine receptor related protein 4; 37845_at Cluster Incl. M58285:Human membrane-associated protein (HEM-1) mRNA, co; 38893_at Cluster Incl. AL008637:Human DNA sequence from clone 833B7 on chromosom; 39581_at Cluster Incl. AA570193:nf38c11.s1 Homo sapiens cDNA /clone=IMAGE-916052; 40310_at Cluster Incl. AF051152:Homo sapiens Toll/interleukin-1 receptor-like pr; 40685_at Cluster Incl. U10868:Human aldehyde dehydrogenase ALDH7 mRNA, complete; 40757_at Cluster Incl. M18737:Human Hanukah factor serine protease (HuHF) mRNA,; 41425_at Cluster Incl. M98833:Human ERGB transcription factor (FLI-1 homolog) mR; 41716_at Cluster Incl. AB020663:Homo sapiens mRNA for KIAA0856 protein, partial; 41868_at Cluster Incl. J04131:Human gamma-glutamyl transpeptidase (GGT) protein; 31845_at Cluster Incl. U32645:Human myeloid elf-1 like factor (MEF) mRNA, comple; 32035_at Cluster Incl. M16942:Human MHC class II HLA-DRw53-associated glycoprote; 32083_at Cluster Incl. AF027826:Homo sapiens putative seven pass transmembrane p; 33238_at Cluster Incl. U23852:Human T-lymphocyte specific protein tyrosine kinas; 33253_at Cluster Incl. D50919:Human mRNA for KIAA0129 gene, complete cds /cds=(1; 33777_at Cluster Incl. D34625:Human TBXAS1 gene for thromboxane synthase, promot; 35172_at Cluster Incl. AF049891:Homo sapiens tyrosylprotein sulfotransferase-2 m; 35995_at Cluster Incl. AF067656:Homo sapiens ZW10 interactor Zwint mRNA, complet; 37565_at Cluster Incl. X85750:H. sapiens mRNA for transcript associated with mono; 37966_at Cluster Incl. AA187563:zp66g11.r1 Homo sapiens cDNA, 5 end /clone=IMAG; 38006_at Cluster Incl. M37766:Human MEM-102 glycoprotein mRNA, complete cds /cds; 38332_at Cluster Incl. U83993:Human P2X4 purinoreceptor mRNA, complete cds /cds=; 39023_at Cluster Incl. AF020038:Homo sapiens NADP-dependent isocitrate dehydroge; 39760_at Cluster Incl. AL031781:dJ51J12.1.3 (human ortholog of mouse KH Domain R; 40407_at Cluster Incl. U28386:Human nuclear localization sequence receptor hSRP1; 40456_at Cluster Incl. AL049963:Homo sapiens mRNA; cDNA DKFZp564A132 (from clone; 32193_at Cluster Incl. AF030339:Homo sapiens receptor for viral semaphorin prote; 33839_at Cluster Incl. D26350:Human mRNA for type 2 inositol 1,4,5-trisphosphate; 34889_at Cluster Incl. AA056747:zk81f02.s1 Homo sapiens cDNA, 3 end /clone=IMAG; 35320_at Cluster Incl. AB004857:Homo sapiens mRNA for NRAMP2, complete cds /cds=; 36576_at Cluster Incl. AF054174:Homo sapiens histone macroH2A1.2 mRNA, complete; 36938_at Cluster Incl. U70063:Human acid ceramidase mRNA, complete cds /cds=(17,; 37363_at Cluster Incl. AB007889:Homo sapiens KIAA0429 mRNA, complete cds /cds=(2; 37684_at Cluster Incl. AB020687:Homo sapiens mRNA for KIAA0880 protein, complete; 38370_at Cluster Incl. U90902:Human clone 23612 mRNA sequence /cds=UNKNOWN /gb=U; 38378_at Cluster Incl. M37033:Human CD53 glycoprotein mRNA, complete cds /cds=(9; 38379_at Cluster Incl. X76534:H. sapiens NMB mRNA /cds=(91,1773) /gb=X76534 /gi=6; 40278_at Cluster Incl. AB029003:Homo sapiens mRNA for KIAA1080 protein, partial; 40571_at Cluster Incl. U90942:Human myosin heavy chain 12 (MYO5A) mRNA, complete; 40585_at Cluster Incl. D25538:Human mRNA for KIAA0037 gene, complete cds /cds=(2; 40910_at Cluster Incl. U56637:Human capping protein alpha subunit isoform 1 mRNA; 41812_s_at Cluster Incl. AB020713:Homo sapiens mRNA for KIAA0906 protein, partia; 41824_at Cluster Incl. AI140114:qa95c06.x1 Homo sapiens cDNA, 3 end /clone=IMAG; 1478_at L10717 /FEATURE= /DEFINITION=HUMTKTCS Homo sapiens T cell-specific tyros; 1405_i_at M21121 /FEATURE= /DEFINITION=HUMTCSM Human T cell-specific protein (RA; 1062_g_at U00672 /FEATURE= /DEFINITION=U00672 Human interleukin-10 receptor mRNA; 461_at U70063 /FEATURE= /DEFINITION=HSU70063 Human acid ceramidase mRNA, complet.

Metagene 119; 36134_at Cluster Incl. U79299:Human neuronal olfactomedin-related ER localized p; 38409_at Cluster Incl. M61199:Human cleavage signal 1 protein mRNA, complete cds; 38737_at Cluster Incl. X57025:Human IGF-I mRNA for insulin-like growth factor I; 1625_at Insulin-Like Growth Factor Ib; 1501_at X57025 /FEATURE=mRNA /DEFINITION=HSIGFACI Human IGF-I mRNA for insulin-1.

Metagene 120; 38210_at Cluster Incl. Z35094:H. sapiens mRNA for SURF-2 /cds=(17,787) /gb=Z35094; 38889_at Cluster Incl. AF104304:Homo sapiens Smad anchor for receptor activation; 39929_at Cluster Incl. AB023139:Homo sapiens mRNA for KIAA0922 protein, partial; 39984_g_at Cluster Incl. U73704:Homo sapiens 48 kDa FKBP-associated protein FAP4; 40758_at Cluster Incl. X81788:Homo sapiens ICT1 (alias DS-1) mRNA /cds=(2,622) /; 41671_at Cluster Incl. U97018:Homo sapiens echinoderm microtubule-associated pro; 32672_at Cluster Incl. AL049387:Homo sapiens mRNA; cDNA DKFZp586N1918 (from clon; 33256_at Cluster Incl. Y14494:Homo sapiens mRNA for mitochondrial carrier protei; 33713_at Cluster Incl. AJ005895:Homo sapiens mRNA for (JM3) preprotein transloca; 33751_at Cluster Incl. AL109702:Homo sapiens mRNA full length insert cDNA clone; 34292_at Cluster Incl. X92475:H. sapiens mRNA for ITBAL protein /cds=(284,1069) /; 35247_at Cluster Incl. AI557062:PT2.1_{—}13_A09.r Homo sapiens cDNA, 3 end /clone_{—}; 35719_at Cluster Incl. AB011178:Homo sapiens mRNA for KIAA0606 protein, partial; 37549_g_at Cluster Incl. U87408:Human clone IMAGE-74593 unknown protein mRNA, pa; 37646_at Cluster Incl. D26018:Human mRNA for KIAA0039 gene, partial cds /cds=(0,; 39394_at Cluster Incl. AF007149:Homo sapiens clone 23568, 23621, 23795, 23873 an; 39705_at Cluster Incl. AB014600:Homo sapiens mRNA for KIAA0700 protein, partial; 39772_at Cluster Incl. AF007157:Homo sapiens clone 23856 unknown mRNA, partial c; 40462_at Cluster Incl. AF055022:Homo sapiens clone 24684 mRNA sequence /cds=UNKN; 40852_at Cluster Incl. AB025254:Homo sapiens mRNA for tudor repeat associator wi; 32246_g_at Cluster Incl. AF014837:Homo sapiens m6A methyltransferase (MT-A70) ge; 32823_at Cluster Incl. W28734:51a1 Homo sapiens cDNA /gb=W28734 /gi=1308682 /ug=; 34848_at Cluster Incl. X69141:H. sapiens mRNA for squalene synthase /cds=(91,1344; 34868_at Cluster Incl. AB029012:Homo sapiens mRNA for KIAA1089 protein, partial; 35274_at Cluster Incl. Y12226:H. sapiens mRNA for gamma-adaptin /cds=(28,2505) /g; 35792_at Cluster Incl. U67963:Human lysophospholipase homolog (HU-K5) mRNA, comp; 36115_at Cluster Incl. L29217:Homo sapiens clk3 mRNA, complete cds /cds=(56,1528; 39557_at Cluster Incl. AI625844:ty65d07.x1 Homo sapiens cDNA, 3 end /clone=IMAG; 40546_s_at Cluster Incl. AF047185:Homo sapiens NADH-ubiquinone oxidoreductase su; 41349_at Cluster Incl. L43964:Homo sapiens (clone F-T03796) STM-2 mRNA, complete; 32558_at Cluster Incl. AB021868:Homo sapiens PIAS3 mRNA for protein inhibitor of; 927_s_at J05582 /FEATURE=mRNA /DEFINITION=HUMPANMU Human pancreatic mucin mRNA,; 101_at Y09305 /FEATURE=cds /DEFINITION=HSDYRK4 H. sapiens mRNA for protein kinase.

Metagene 121; 41871_at Cluster Incl. AI660929:wf20a09.x1 Homo sapiens cDNA, 3 end /clone=IMAG; 35208_at Cluster Incl. AB020681:Homo sapiens mRNA for KIAA0874 protein, partial; 35622_at Cluster Incl. AB001451:Homo sapiens mRNA for Sck, partial cds /cds=(0,1; 36899_at Cluster Incl. M97287:Human MAR/SAR DNA binding protein (SATB1) mRNA, co; 38250_at Cluster Incl. D26488:Human mRNA for KIAA0007 gene, partial cds /cds=(0,; 41167_at Cluster Incl. M64929:Human protein phosphatase 2A alpha subunit mRNA, c; 34316_at Cluster Incl. W52024:zd13a03.s1 Homo sapiens cDNA, 3 end /clone=IMAGE-; 38013_at Cluster Incl. AL096842:Homo sapiens mRNA; cDNA DKFZp586D1519 (from clon; 40203_at Cluster Incl. AJ012375:Homo sapiens mRNA for SUI1 protein translation i; 1226_at U69611 /FEATURE= /DEFINITION=HSU69611 Human TNF-alpha converting enzyme.

Metagene 122; 35489_at Cluster Incl. M82962:Human N-benzoyl-L-tyrosyl-p-amino-benzoic acid hyd; 447_g_at U89896 /FEATURE= /DEFINITION=HSU89896 Homo sapiens casein kinase I gamm; 372_f_at Z84718 /FEATURE=cds#2 /DEFINITION=HS322B1 Human DNA sequence from clone.

Metagene 123; 34959_at Cluster Incl. M15059:Human Fc-epsilon receptor (IgE receptor) mRNA, com; 38879_at Cluster Incl. D83664:Human mRNA for CAAF1 (calcium-binding protein in a; 41365_at Cluster Incl. Y09788:H. sapiens MUC5B gene /cds=(0,2538) /gb=Y09788 /gi=; 34319_at Cluster Incl. AA131149:zo16d05.r1 Homo sapiens cDNA, 5 end /clone=IMAG; 1352_at U11870 /FEATURE=mRNA /DEFINITION=HSU11870 Human interleukin-8 receptor t.

Metagene 124; 35951_at Cluster Incl. AB018286:Homo sapiens mRNA for KIAA0743 protein, complete; 37459_at Cluster Incl. X57527:Human COL8A1 mRNA for alpha 1(VIII) collagen /cds=; 41864_at Cluster Incl. AF052181:Homo sapiens clone 24790 mRNA sequence /cds=UNKN; 36853_at Cluster Incl. M64752:Human glutamate receptor subunit (GluH1) mRNA, com; 37628_at Cluster Incl. M69177:Human monoamine oxidase B (MAOB) mRNA, complete cd; 40078_at Cluster Incl. AF015287:Homo sapiens serine protease mRNA, complete cds; 33431_at Cluster Incl. U05291:Human fibromodulin mRNA, partial cds /cds=(0,177); 35752_s_at Cluster Incl. M15036:Human vitamin K-dependent plasma protein S mRNA,; 40936_at Cluster Incl. AI651806:wb55f10.x1 Homo sapiens cDNA, 3 end /clone=IMAG; 41294_at Cluster Incl. AJ238246:Homo sapiens mRNA for sarcolectin /cds=(61,1470); 32593_at Cluster Incl. D42043:Human mRNA for KIAA0084 gene, partial cds /cds=(0,; 2073_s_at L34058 /FEATURE= /DEFINITION=HUMCA13A Homo sapiens cadherin-13 mRNA, c; 482_at U59289 /FEATURE= /DEFINITION=HSU59289 Human H-cadherin mRNA, complete cds; 483_g_at U59289 /FEATURE= /DEFINITION=HSU59289 Human H-cadherin mRNA, complete c.

Metagene 125; 31670_s_at Cluster Incl. U81554:Homo sapiens CaM kinase II isoform mRNA, complet; 31987_at Cluster Incl. AL049268:Homo sapiens mRNA; cDNA DKFZp564G103 (from clone; 33988_at Cluster Incl. X75861:H. sapiens TEGT gene /cds=(40,753) /gb=X75861 /gi=4; 39607_at Cluster Incl. AL080178:Homo sapiens mRNA; cDNA DKFZp434K171 (from clone; 39663_at Cluster Incl. D63998:Human mRNA for golgi alpha-mannosidaseII, complete; 33302_at Cluster Incl. AF016028:Homo sapiens sarcospan-2 (SPN2) mRNA, complete c; 37264_at Cluster Incl. U09410:Human zinc finger protein ZNF131 mRNA, partial cds; 37608_g_at Cluster Incl. AJ005168:Homo sapiens KHK gene, exons 4-8 /cds=(0,552); 38650_at Cluster Incl. L27560:Human insulin-like growth factor binding protein 5; 38673_s_at Cluster Incl. D64137:Human KIP2 gene for Cdk-inhibitor p57KIP2, compl; 39327_at Cluster Incl. D86983:Human mRNA for KIAA0230 gene, partial cds /cds=(0,; 38841_at Cluster Incl. AF068195:Homo sapiens putative glialblastoma cell differe; 39561_at Cluster Incl. AL008583:dJ327J16.3 (novel CHROMObox family protein) /cds; 40275_at Cluster Incl. AL046322:DKFZp434I087_r1 Homo sapiens cDNA, 5 end /clone; 40552_s_at Cluster Incl. AL049987:Homo sapiens mRNA; cDNA DKFZp564F112 (from clo; 41293_at Cluster Incl. AI123710:oo16h01.x1 Homo sapiens cDNA, 3 end /clone=IMAG; 41829_at Cluster Incl. AB018274:Homo sapiens mRNA for KIAA0731 protein, partial; 2050_s_at M29870 /FEATURE= /DEFINITION=HUMRACA Human ras-related C3 botulinum to; 1677_at M65062 /FEATURE= /DEFINITION=HUMIGFBP6 Human insulin-like growth factor; 1396_at L27560 /FEATURE=mRNA /DEFINITION=HUMIGFBP5X Human insulin-like growth fa; 1114_at M22490 /FEATURE= /DEFINITION=HUMBMP2B Human bone morphogenetic protein-2.

Metagene 126; 34928_at Cluster Incl. AF060865:Homo sapiens chromosome 16 zinc finger protein Z; 40377_at Cluster Incl. AB014582:Homo sapiens mRNA for KIAA0682 protein, complete; 32661_s_at Cluster Incl. D79992:Human mRNA for KIAA0170 gene, complete cds /cds=; 33732_at Cluster Incl. Y08387:H. sapiens mRNA for mu-ARP2 protein /cds=(54,1415); 39022_at Cluster Incl. AL050110:Homo sapiens mRNA; cDNA DKFZp586J0619 (from clon; 40833_r_at Cluster Incl. AL050126:Homo sapiens mRNA; cDNA DKFZp586G011 (from clo; 38020_at Cluster Incl. AB014552:Homo sapiens mRNA for KIAA0652 protein, complete; 38109_at Cluster Incl. AF020544:Homo sapiens inactive palmitoyl-protein thioeste; 33111_at Cluster Incl. AI333845:qp99a05.x1 Homo sapiens cDNA, 3 end /clone=IMAG; 942_at D31815 /FEATURE= /DEFINITION=HUMSMP30 Human mRNA for SMP-30 (senescence m.

Metagene 127; 34480_at Cluster Incl. AF016272:Homo sapiens Ksp-cadherin (CDH16) mRNA, complete; 40366_at Cluster Incl. M25322:Human granule membrane protein-140 mRNA, complete; 33284_at Cluster Incl. M19507:Human myeloperoxidase mRNA, complete cds /cds=UNKN; 38284_at Cluster Incl. AJ007041:Homo sapiens mRNA for trithorax homologue 2 /cds; 34303_at Cluster Incl. AL049949:Homo sapiens mRNA; cDNA DKFZp564L0822 (from clon; 36150_at Cluster Incl. AB020649:Homo sapiens mRNA for KIAA0842 protein, partial; 37760_at Cluster Incl. AB015019:Homo sapiens mRNA for BAP2-alpha protein, comple; 38064_at Cluster Incl. X79882:H. sapiens lrp mRNA /cds=(105,2795) /gb=X79882 /gi=; 39119_s_at Cluster Incl. AA631972:fmfc39 Homo sapiens cDNA /clone=CR7-5 /gb=AA63; 1196_at D00591 /FEATURE=exons#7-14 /DEFINITION=HUMRCC1 Homo sapiens RCC1 gene, e.

Metagene 128; 32447_at Cluster Incl. U76388:Human steroidogenic factor 1 mRNA, complete cds /c; 35519_at Cluster Incl. AL049431:Homo sapiens mRNA; cDNA DKFZp586J211 (from clone; 35864_at Cluster Incl. Y00970:Human mRNA for acrosin (EC 3.4.21.10) /cds=(16,128; 35896_at Cluster Incl. D87002:Human (lambda) DNA for immunoglobin light chain /c; 34280_at Cluster Incl. Y09765:Homo sapiens mRNA for putative GABA receptor epsil; 35152_at Cluster Incl. AJ001016:Homo sapiens mRNA encoding RAMP3 /cds=(29,475) /; 37272_at Cluster Incl. X57206:H. sapiens mRNA for 1D-myo-inositol-trisphosphate 3; 38269_at Cluster Incl. AL050147:Homo sapiens mRNA; cDNA DKFZp586E0820 (from clon; 38628_at Cluster Incl. AF029777:Homo sapiens histone acetyltransferase (GCN5) mR; 39358_at Cluster Incl. U37146:Human silencing mediator of retinoid and thyroid h; 41203_at Cluster Incl. L49380:Homo sapiens clone B4 transcription factor ZFM1 mR; 33454_at Cluster Incl. AF016903:Homo sapiens agrin precursor mRNA, partial cds /; 33916_at Cluster Incl. AB023192:Homo sapiens mRNA for KIAA0975 protein, partial; 37365_at Cluster Incl. X63368:H. sapiens HSJ1 mRNA /cds=(25,1080) /gb=X63368 /gi=; 37687_i_at Cluster Incl. M31932:Human IgG low affinity Fc fragment receptor (FcR; 32592_at Cluster Incl. AB002321:Human mRNA for KIAA0323 gene, partial cds /cds=(; 1988_at X76079 /FEATURE=exons#1-4 /DEFINITION=HSPDGF H. sapiens mRNA for platelet; 1664_at Insulin-Like Growth Factor 2; 1119_at J05249 /FEATURE= /DEFINITION=HUMREPA Human replication protein A 32-kDa; 448_s_at U93237 /FEATURE=mRNA#1 /DEFINITION=HSU93237 Human menin (MEN1) gene, co; 181_g_at S82470 /FEATURE= /DEFINITION=S82470 BB1=malignant cell expression-enhan.

Metagene 129; 34993_at Cluster Incl. X95191:H. sapiens mRNA for delta-sarcoglycan /cds=(0,872); 38965_at Cluster Incl. M55172:Human large aggregating cartilage proteoglycan cor; 39206_s_at Cluster Incl. X17406:Human mRNA for cartilage specific proteoglycan /; 39207_r_at Cluster Incl. X17406:Human mRNA for cartilage specific proteoglycan /; 39250_at Cluster Incl. X96584:H. sapiens mRNA for NOV protein /cds=(72,1145) /gb=; 39616_at Cluster Incl. AL050227:Homo sapiens mRNA; cDNA DKFZp586M0723 (from clon; 39966_at Cluster Incl. AF059274:Homo sapiens neuroglycan C mRNA, complete cds /c; 31888_s_at Cluster Incl. AF001294:Homo sapiens IPL (IPL) mRNA, complete cds /cds; 32687_s_at Cluster Incl. X83857:H. sapiens mRNA for prostaglandin E receptor (EP3; 34285_at Cluster Incl. AB018338:Homo sapiens mRNA for KIAA0795 protein, partial; 35729_at Cluster Incl. AB018270:Homo sapiens mRNA for KIAA0727 protein, partial; 38643_at Cluster Incl. W87466:zh67c05.s1 Homo sapiens cDNA, 3 end /clone=IMAGE-; 39063_at Cluster Incl. J00073:Human alpha-cardiac actin gene, 5 flank and /cds=(; 41126_at Cluster Incl. AA978353:oq40b07.s1 Homo sapiens cDNA, 3 end /clone=IMAG; 41758_at Cluster Incl. AL096879:Novel human mRNA similar to C. elegans gene WP-C; 32182_at Cluster Incl. AB023182:Homo sapiens mRNA for KIAA0965 protein, partial; 32838_at Cluster Incl. S67247:smooth muscle myosin heavy chain isoform SMemb [hu; 35833_at Cluster Incl. AL080184:Homo sapiens mRNA; cDNA DKFZp434O071 (from clone; 37658_at Cluster Incl. L13720:Homo sapiens growth-arrest-specific protein (gas); 38034_at Cluster Incl. M16505:Human steroid sulfatase (STS) mRNA, complete cds /; 1424_s_at D78577 /FEATURE=expanded_cds /DEFINITION=D78576S2 Human DNA for 14-3-3.

Metagene 130; 33004_g_at Cluster Incl. AI275502:ql74d06.x1 Homo sapiens cDNA, 3 end /clone=IM; 36767_at Cluster Incl. K03191:Human cytochrome P-1-450 (TCDD-inducible) mRNA, co; 32714_s_at Cluster Incl. L17075:Human TGF-b superfamily receptor type I mRNA, co; 41260_at Cluster Incl. U59321:Human DEAD-box protein p72 (P72) mRNA, complete cd; 1025_g_at X02612 /FEATURE=expanded_cds /DEFINITION=HSCYP450 Human gene for cytoc.

Metagene 131; 31457_at Cluster Incl. AF042832:Homo sapiens forkhead-related transcription fact; 38223_at Cluster Incl. AB024057:Homo sapiens mRNA for vascular Rab-GAP/TBC-conta; 40348_s_at Cluster Incl. W25866:14c12 Homo sapiens cDNA /gb=W25866 /gi=1305989 /; 39387_at Cluster Incl. U34044:Human selenium donor protein (selD) mRNA, complete; 40092_at Cluster Incl. AB002312:Human mRNA for KIAA0314 gene, partial cds /cds=(; 32248_at Cluster Incl. AL045811:DKFZp434H166_r1 Homo sapiens cDNA, 5 end /clone; 1217_gnat X07109 /FEATURE=cds /DEFINITION=HSPKCB2A Human mRNA for protein kinase.

Metagene 132; 39392_at Cluster Incl. AJ002190:Homo sapiens cDNA for dihydroxyacetone phosphate; 41196_at Cluster Incl. L38951:Homo sapiens importin beta subunit mRNA, complete; 37650_at Cluster Incl. U41315:Human ring zinc-finger protein (ZNF127-Xp) gene an; 37725_at Cluster Incl. X74008:H. sapiens mRNA for protein phosphatase 1 gamma /cd; 41243_at Cluster Incl. AB007916:Homo sapiens mRNA for KIAA0447 protein, complete; 1239_s_at U13021 /FEATURE= /DEFINITION=HSU13021 Human positive regulator of prog; 555_at U05227 /FEATURE= /DEFINITION=HSU05227 Human Rar protein mRNA, complete cd.

Metagene 133; 31680_at Cluster Incl. M55630:Human topoisomerase I pseudogene 2 /cds=UNKNOWN /g; 38545_at Cluster Incl. M31682:Human testicular inhibin beta-B-subunit mRNA, 3 e; 40735_at Cluster Incl. D16626:Human mRNA for histidase, complete cds /cds=(243,2; 41100_at Cluster Incl. AB023172:Homo sapiens mRNA for KIAA0955 protein, complete; 41460_at Cluster Incl. AF080561:Homo sapiens SYT interacting protein SIP mRNA, c; 31802_at Cluster Incl. D86979:Human mRNA for KIAA0226 gene, complete cds /cds=(6; 32644_at Cluster Incl. D79991:Human mRNA for KIAA0169 gene, partial cds /cds=(0,; 34765_at Cluster Incl. D13645:Human mRNA for KIAA0020 gene, complete cds /cds=(4; 39755_at Cluster Incl. Z93930:Human DNA sequence from clone 292E10 on chromosome; 40050_at Cluster Incl. AF069747:Homo sapiens MTG8-like protein MTGR1a mRNA, comp; 32245_at Cluster Incl. AF014837:Homo sapiens m6A methyltransferase (MT-A70) gene; 32820_at Cluster Incl. U71267:Human potential transcriptional repressor NOT4Hp (; 35351_at Cluster Incl. U89505:Human Hlark mRNA, complete cds /cds=(55,1155) /gb=; 35830_at Cluster Incl. AB002368:Human mRNA for KIAA0370 gene, partial-cds /cds=(; 37676_at Cluster Incl. AF056490:Homo sapiens cAMP-specific phosphodiesterase 8A; 40891_f_at Cluster Incl. X92896:H. sapiens mRNA for ITBA2 protein /cds=(10,327) /; 40946_at Cluster Incl. AI023044:ow65c01.s1 Homo sapiens cDNA, 3 end /clone=IMAG; 1248_at U37689 /FEATURE= /DEFINITION=HSU37689 Human RNA polymerase II subunit (h; 776_at D13435 /FEATURE= /DEFINITION=HUMPIGF Human mRNA for PIG-F (phosphatidyl-i.

Metagene 134; 31562_at Cluster Incl. U63973:Human rhodopsin kinase mRNA, complete cds /cds=(10; 32364_at Cluster Incl. AJ011785:Homo sapiens mRNA for Six9 protein /cds=(74,814); 34467_g_at Cluster Incl. Y12505:H. sapiens mRNA for serotonin receptor 5-HT4B, sp; 35403_at Cluster Incl. AB029017:Homo sapiens mRNA for KIAA1094 protein, complete; 37832_at Cluster Incl. AL080062:Homo sapiens mRNA; cDNA DKFZp564I122 (from clone; 38215_at Cluster Incl. U84894:Human 239AB mRNA, complete cds /cds=(114,1028) /gb; 40024_at Cluster Incl. D86640:Homo sapiens mRNA for stac, complete cds /cds=(39,; 31812_at Cluster Incl. M24470:Human glucose-6-phosphate dehydrogenase, complete; 33277_at Cluster Incl. AB028996:Homo sapiens mRNA for KIAA1073 protein, complete; 35151_at Cluster Incl. AF089814:Homo sapiens growth suppressor related (DOC-1R); 36855_r_at Cluster Incl. M81886:Human glutamate receptor type 1 (HBGR1) mRNA, co; 37232_at Cluster Incl. AB011158:Homo sapiens mRNA for KIAA0586 protein, complete; 37284_at Cluster Incl. U60800:Human semaphorin (CD100) mRNA, complete cds /cds=(; 38258_at Cluster Incl. U79290:Human clone 23908 mRNA sequence /cds=UNKNOWN /gb=U; 41127_at Cluster Incl. L14595:Human alanine/serine/cysteine/threonine transporte; 41143_at Cluster Incl. U12022:Human calmodulin (CALM1) gene /cds=(199,648) /gb=U; 33388_at Cluster Incl. AL080223:Homo sapiens mRNA; cDNA DKFZp566H2446 (from clon; 36593_at Cluster Incl. U67368:Human multiple-exostosis 2 (EXT2) gene /cds=(30,21; 38785_at Cluster Incl. X52228:Human mRNA for secreted epithelial tumour mucin an; 39836_at Cluster Incl. AI925231:wn52c05.x1 Homo sapiens cDNA, 3 end /clone=IMAG; 41358_at Cluster Incl. AI827730:wf11d12.x1 Homo sapiens cDNA, 3 end /clone=IMAG; 41598_at Cluster Incl. AA890010:aj89h08.s1 Homo sapiens cDNA, 3 end /clone=IMAG; 1898_at L24203 /FEATURE= /DEFINITION=HUMDK Homo sapiens ataxia-telangiectasia gr; 829_s_at U21689 /FEATURE=cds /DEFINITION=HSU21689 Human glutathione S-transferas; 114_r_at X14474 /FEATURE=cds /DEFINITION=HSTAUI Human mRNA for microtubule-assoc.

Metagene 135; 33684_at Cluster Incl. Z71621:H. sapiens Wnt-13 mRNA /cds=(491,1609) /gb=Z71621 /; 37070_at Cluster Incl. D14720:Homo sapiens gene for peripheral myelin protein ze; 38179_at Cluster Incl. U31248:Human zinc finger protein (ZNF174) mRNA, complete; 39661_s_at Cluster Incl. AF034102:Homo sapiens NBMPR-insensitive nucleoside tran; 36872_at Cluster Incl. AL120559:DKFZp761B219_r1 Homo sapiens cDNA, 5 end /clone; 41748_at Cluster Incl. AA196476:zp99g10.r1 Homo sapiens cDNA, 5 end /clone=IMAG; 37758_s_at Cluster Incl. W28479:47d8 Homo sapiens cDNA /gb=W28479 /gi=1308427 /u.

Metagene 136; 33462_at Cluster Incl. D13626:Human mRNA for KIAA0001 gene, complete cds /cds=(2; 36328_at Cluster Incl. M31651:Homo sapiens sex hormone-binding globulin (SHBG) g; 32032_at Cluster Incl. L77566:Homo sapiens DGS-I mRNA, 3 end /cds=UNKNOWN /gb=L; 35173_at Cluster Incl. U03886:Human GS2 mRNA, complete cds /cds=(130,891) /gb=U0; 36012_at Cluster Incl. Y09631:H. sapiens mRNA for PIBF1 protein, complete /cds=(0; 36641_at Cluster Incl. U03851:Human capping protein alpha mRNA, partial cds /cds.

Metagene 137; 33505_at Cluster Incl. AI887421:wm05c01.x1 Homo sapiens cDNA, 3 end /clone=IMAG; 36067_at Cluster Incl. AB000887:Homo sapiens mRNA for EBI1-ligand chemokine, com; 39781_at Cluster Incl. U20982:Human insulin-like growth factor binding protein-4; 36686_at Cluster Incl. U07919:Human aldehyde dehydrogenase 6 mRNA, complete cds; 37017_at Cluster Incl. M22430:Human RASF-A PLA2 mRNA, complete cds /cds=(135,569; 1737_s_at M62403 /FEATURE= /DEFINITION=HUMIGFBP5 Human insulin-like growth facto; 1042_at U27185 /FEATURE= /DEFINITION=HSU27185 Human RAR-responsive (TIG1) mRNA,; 614_at M22430 /FEATURE= /DEFINITION=HUMRASFAB Human RASF-A PLA2 mRNA, complete c.

Metagene 138; 34034_at Cluster Incl. D80011:Human mRNA for KIAA0189 gene, complete cds /cds=(3; 34965_at Cluster Incl. AF031824:Homo sapiens leukocystatin mRNA, complete cds /c; 38956_at Cluster Incl. AF052111:Homo sapiens clone 23953 mRNA sequence /cds=UNKN; 41471_at Cluster Incl. W72424:zd66a09.s1 Homo sapiens cDNA, 3 end /clone=IMAGE-; 32096_at Cluster Incl. AC005546:Homo sapiens chromosome 19, cosmid R29425 /cds=(; 33804_at Cluster Incl. U43522:Human cell adhesion kinase beta (CAKbeta) mRNA, co; 35714_at Cluster Incl. U89606:Human pyridoxal kinase mRNA, complete cds /cds=(6,; 36856_at Cluster Incl. W28743:51a9 Homo sapiens cDNA /gb=W28743 /gi=1308691 /ug=; 37180_at Cluster Incl. X14034:Human mRNA for phospholipase C /cds=(152,3910) /gb; 37188_at Cluster Incl. X92720:H. sapiens mRNA for phosphoenolpyruvate carboxykina; 37645_at Cluster Incl. Z22576:H. sapiens CD69 gene /cds=(81,680) /gb=Z22576 /gi=3; 39385_at Cluster Incl. M22324:Human aminopeptidase N/CD13 mRNA encoding aminopep; 40772_at Cluster Incl. AA284298:zc30c10.T7 Homo sapiens cDNA, 3 end /clone=IMAG; 35287_at Cluster Incl. AF046888:Homo sapiens proliferation inducing ligand APRIL; 37033_s_at Cluster Incl. X13710:H. sapiens unspliced mRNA for glutathione peroxid; 37351_at Cluster Incl. X90858:H. sapiens mRNA for uridine phosphorylase /cds=(352; 1780_at M19722 /FEATURE= /DEFINITION=HUMFGR Human fgr proto-oncogene encoded p55; 1637_at U09578 /FEATURE= /DEFINITION=HSU09578 Homo sapiens MAPKAP kinase (3pK) m; 1426_at D89077 /FEATURE= /DEFINITION=D89077 Homo sapiens mRNA for Src-like adapt; 1427_g_at D89077 /FEATURE= /DEFINITION=D89077 Homo sapiens mRNA for Src-like ada; 868_at U13991 /FEATURE= /DEFINITION=HSU13991 Human TATA-binding protein associat; 794_at X62055 /FEATURE=cds /DEFINITION=HSPTP1C H. sapiens PTP1C mRNA for protein-; 307_at J03600 /FEATURE= /DEFINITION=HUMLOX5 Human lipoxygenase mRNA, complete cd; 182_at U01062 /FEATURE=mRNA /DEFINITION=HUMIP3R3 Human type 3 inositol 1,4,5-tri.

Metagene 139; 38501_s_at Cluster Incl. U37139:Human beta 3-endonexin mRNA, long form and short; 33234_at Cluster Incl. AA887480:oj54a12.s1 Homo sapiens cDNA, 3 end /clone=IMAG; 33753_at Cluster Incl. AB014566:Homo sapiens mRNA for KIAA0666 protein, partial; 35215_at Cluster Incl. AL049996:Homo sapiens mRNA; cDNA DKFZp564K112 (from clone; 36456_at Cluster Incl. AL080063:Homo sapiens mRNA; cDNA DKFZp564I052 (from clone; 37590_g_at Cluster Incl. AL109698:Homo sapiens mRNA full length insert cDNA clon; 39373_at Cluster Incl. AF035284:Homo sapiens clone 23716 mRNA sequence /cds=UNKN; 34314_at Cluster Incl. X59543:Human mRNA for M1 subunit of ribonucleotide reduct; 35352_at Cluster Incl. AB002305:Human mRNA for KIAA0307 gene, complete cds /cds=; 35804_at Cluster Incl. AB022785:Homo sapiens ASH2L gene, complete cds, similar t; 37040_at Cluster Incl. D42041:Human mRNA for KIAA0088 gene, partial cds /cds=(0,.

Metagene 140; 34433_at Cluster Incl. AF035299:Homo sapiens clone 23863 mRNA, partial cds /cds=; 36136_at Cluster Incl. AF010315:Homo sapiens Pig11 (PIG11) mRNA, complete cds /c; 32513_at Cluster Incl. W27495:31h12 Homo sapiens cDNA /gb=W27495 /gi=1307317 /ug; 1386_at M83738 /FEATURE= /DEFINITION=HUMPTPSA Human protein-tyrosine phosphatase; 141_s_at U75276 /FEATURE= /DEFINITION=HSU75276 Human TFIIB related factor hBRF (.

Metagene 141; 33058_at Cluster Incl. Y17282:Homo sapiens mRNA for cytokeratin type II /cds=(18; 40750_at Cluster Incl. U18088:Human 3,5-cyclic AMP phosphodiesterase inactive s; 38636_at Cluster Incl. AB003184:Homo sapiens mRNA for ISLR, complete cds /cds=(9; 39075_at Cluster Incl. AF040958:Homo sapiens lysosomal neuraminidase precursor,; 39395_at Cluster Incl. AA704137:ag47g01.s1 Homo sapiens cDNA, 3 end /clone=IMAG; 38780_at Cluster Incl. J04794:Human aldehyde reductase mRNA, complete cds /cds=(; 41584_at Cluster Incl. AF062529:Homo sapiens clone 486790 diphosphoinositol poly; 1830_s_at M38449 /FEATURE= /DEFINITION=HUMTGFBA Human transforming growth factor; 1747_at AD000092 /FEATURE=cds#2 /DEFINITION=CH19HHR23 Homo sapiens DNA from chro.

Metagene 142; 33537_at Cluster Incl. X90761:Homo sapiens hHa2 gene /cds=(61,1407) /gb=X90761 /; 38576_at Cluster Incl. AJ223353:Homo sapiens mRNA for histone H2B, clone pJG4-5-; 32627_at Cluster Incl. AF039023:Homo sapiens Ran-GTP binding protein mRNA, parti; 32718_at Cluster Incl. AF038009:Homo sapiens tyrosylprotein sulfotransferase-1 m; 32618_at Cluster Incl. X93086:H. sapiens mRNA for biliverdin IX alpha reductase /.

Metagene 143; 31319_at Cluster Incl. M20707:Human kappa-immunoglobulin germline pseudogene (Ch; 31586_f_at Cluster Incl. X72475:H. sapiens mRNA for rearranged Ig kappa light cha; 34094_i_at Cluster Incl. U80114:Human immunoglobulin heavy chain variable region; 34095_f_at Cluster Incl. U80114:Human immunoglobulin heavy chain variable region; 34098_f_at Cluster Incl. AI799757:wc37g12.x1 Homo sapiens cDNA, 3 end /clone=IM; 35530_f_at Cluster Incl. X92997:H. sapiens mRNA for IgG lambda light chain V-J-C; 35566_f_at Cluster Incl. AF015128:Homo sapiens IgG heavy chain variable region (; 36277_at Cluster Incl. M23323:Human membrane protein (CD3-epsilon) gene /cds=(59; 37061_at Cluster Incl. U29615:Human chitotriosidase precursor mRNA, complete cds; 40749_at Cluster Incl. X07203:Human mRNA for CD20 receptor (S7) /cds=(90,983) /g; 40159_r_at Cluster Incl. M55067:Human 47-kD autosomal chronic granulomatous dise; 38017_at Cluster Incl. U05259:Human MB-1 gene, complete cds /cds=(36,716) /gb=U0; 38018_g_at Cluster Incl. U05259:Human MB-1 gene, complete cds /cds=(36,716) /gb=; 41585_at Cluster Incl. AB018289:Homo sapiens mRNA for KIAA0746 protein, partial; 1633_g_at U77735 /FEATURE= /DEFINITION=HSU77735 Human pim-2 protooncogene homolo.

Metagene 144; 39972_at Cluster Incl. Z94155:H. sapiens mRNA for P2Y-like G-protein coupled rece; 34281_at Cluster Incl. AF039555:Homo sapiens visinin-like protein 1 (VSNL1) mRNA; 35658_at Cluster Incl. U40380:Human presenilin 1-374 (AD3-212) mRNA, complete cd; 35789_at Cluster Incl. AB028965:Homo sapiens mRNA for KIAA1042 protein, complete; 38467_at Cluster Incl. U96721:Homo sapiens alternative Hermansky-Pudlak syndrome; 32606_at Cluster Incl. AA135683:zl10c08.r1 Homo sapiens cDNA, 5 end /clone=IMAG; 1378_g_at M58603 /FEATURE= /DEFINITION=HUMNFKB Human nuclear factor kappa-B DNA.

Metagene 145; 31378_at Cluster Incl. AF041339:Homo sapiens homeodomain protein (PITX3) mRNA, c; 35537_at Cluster Incl. AF029761:Homo sapiens decoy receptor 2 mRNA, complete cds; 35448_at Cluster Incl. U37219:Human cyclophilin-like protein CyP-60 mRNA, comple; 38191_at Cluster Incl. AI040181:ox42d12.s1 Homo sapiens cDNA, 3 end /clone=IMAG; 41445_at Cluster Incl. X02812:Human mRNA for transforming growth factor-beta (TG; 33719_at Cluster Incl. AF010242:AF010242 Homo sapiens cDNA /gb=AF010242 /gi=2612; 34367_at Cluster Incl. AF006043:Homo sapiens 3-phosphoglycerate dehydrogenase mR; 39520_at Cluster Incl. AI924382:wn60d01.x1 Homo sapiens cDNA, 3 end /clone=IMAG; 41541_at Cluster Incl. AI971642:wr06e07.x1 Homo sapiens cDNA, 3 end /clone=IMAG; 33151_s_at Cluster Incl. W25932:15b1 Homo sapiens cDNA /gb=W25932 /gi=1306055 /u; 2066_at L22474 /FEATURE= /DEFINITION=HUMBAXB Human Bax beta mRNA, complete cds; 2023_g_at M77198 /FEATURE= /DEFINITION=HUMRPKB Human rac protein kinase beta mRN; 2034_s_at U10906 /FEATURE= /DEFINITION=HSU10906 Human cyclin-dependent kinase in; 1950_s_at AB004922 /FEATURE=cds /DEFINITION=AB004922S1 Homo sapiens gene for Sma; 1542_at X04571 /FEATURE=cds /DEFINITION=HSEGFRER Human mRNA for kidney epidermal; 1259_at L76568 /FEATURE=exons#13-14 /DEFINITION=HUMERCC4G Homo sapiens excision; 1235_at M86400 /FEATURE= /DEFINITION=HUMPHPLA2 Human phospholipase A2 mRNA, comp; 1155_at J03069 /FEATURE=mRNA /DEFINITION=HUMMYCL2A Human MYCL2 gene, complete cd; 1034_at U14394 /FEATURE= /DEFINITION=HSU14394 Human tissue inhibitor of metallop; 438_at X07767 /FEATURE=cds /DEFINITION=HSPKA Human mRNA for cAMP-dependent prote; 104_at Z21966 /FEATURE=cds /DEFINITION=HSMPOUHOX H. sapiens mPOU homeobox protein.

Metagene 146; 35060_at Cluster Incl. U51224:Human U2AFBPL gene, complete cds /cds=(111,1550) /; 34027_f_at Cluster Incl. AA010078:ze16d01.s1 Homo sapiens cDNA, 3 end /clone=IM; 39979_at Cluster Incl. L29433:Human factor X (blood coagulation factor) gene /cd; 40644_g_at Cluster Incl. M34480:Human platelet glycoprotein IIb (GPIIb) mRNA, co; 33314_at Cluster Incl. U69141:Human glutaryl-CoA dehydrogenase mRNA, complete cd; 36543_at Cluster Incl. J02931:Human placental tissue factor (two forms) mRNA, co; 38649_at Cluster Incl. AB023187:Homo sapiens mRNA for KIAA0970 protein, complete; 41158_at Cluster Incl. M54927:Human myelin proteolipid protein mRNA, complete cd; 38057_at Cluster Incl. AL049798:Human DNA sequence from clone 797M17 on chromoso; 38059_g_at Cluster Incl. Z22865:H. sapiens dermatopontin mRNA, complete CDS /cds=; 39893_at Cluster Incl. AB010414:Homo sapiens mRNA for G-protein gamma 7, complet; 40185_at Cluster Incl. S40719:glial fibrillary acidic protein [human, glioma cel.

Metagene 147; 31637_s_at Cluster Incl. X72631:H. sapiens mRNA encoding Rev-ErbAalpha /cds=UNKNO; 36780_at Cluster Incl. M25915:Human complement cytolysis inhibitor (CLI) mRNA, c; 41039_at Cluster Incl. AL022476:dJ323M22.2.1 (novel protein similar to KIAA0173; 31896_at Cluster Incl. AL050281:Homo sapiens mRNA; cDNA DKFZp586G1219 (from clon; 32117_at Cluster Incl. U51698:HSU51698 Homo sapiens cDNA /gb=U51698 /gi=1255268; 33810_at Cluster Incl. AF110377:Homo sapiens PCAF-associated factor 400 (PAF400); 36003_at Cluster Incl. AJ005698:Homo sapiens mRNA for poly(A)-specific ribonucle; 36545_s_at Cluster Incl. AB011114:Homo sapiens mRNA for KIAA0542 protein, comple; 37956_at Cluster Incl. U37519:Human aldehyde dehydrogenase (ALDH8) mRNA, complet; 38719_at Cluster Incl. U03985:Human N-ethylmaleimide-sensitive factor mRNA, part; 39370_at Cluster Incl. W28807:52a3 Homo sapiens cDNA /gb=W28807 /gi=1308755 /ug=; 39404_s_at Cluster Incl. D86988:Human mRNA for KIAA0221 gene, complete cds /cds=; 40498_g_at Cluster Incl. AF040707:Homo sapiens candidate tumor suppressor gene 2; 40787_at Cluster Incl. U90911:Human clone 23652 mRNA sequence /cds=UNKNOWN /gb=U; 33920_at Cluster Incl. AF051782:Homo sapiens diaphanous 1 (HDIA1) mRNA, complete; 34380_at Cluster Incl. AC004472:Homo sapiens chromosome 9, P1 clone 11659 /cds=(; 34396_at Cluster Incl. AB023195:Homo sapiens mRNA for KIAA0978 protein, partial; 35270_at Cluster Incl. W16505:zb05e12.r1 Homo sapiens cDNA, 5 end /clone=IMAGE-; 35301_at Cluster Incl. AL049941:Homo sapiens mRNA; cDNA DKFZp564E2222 (from clon; 35323_at Cluster Incl. U78525:Homo sapiens eukaryotic translation initiation fac; 36996_at Cluster Incl. U41635:Human OS-9 precurosor mRNA, complete cds /cds=(85,; 38423_at Cluster Incl. L38935:Homo sapiens GT212 mRNA /cds=UNKNOWN /gb=L38935 /g; 33198_at Cluster Incl. AA206524:zq58b03.r1 Homo sapiens cDNA, 5 end /clone=IMAG.

Metagene 148; 32915_at Cluster Incl. AL109730:Homo sapiens mRNA full length insert cDNA clone; 33546_at Cluster Incl. AI923984:wn49d12.x1 Homo sapiens cDNA, 3 end /clone=IMAG; 33571_at Cluster Incl. X80590:H. sapiens PHKG1 mRNA /cds=(119,1282) /gb=X80590 /g; 35378_at Cluster Incl. AI051683:oy77h08.x1 Homo sapiens cDNA, 3 end /clone=IMAG; 41641_at Cluster Incl. AJ223603:Homo sapiens mRNA encoding rat C4.4-like protein; 33266_at Cluster Incl. AF015254:Homo sapiens serine/threonine kinase (STK-1) mRN; 37278_at Cluster Incl. X92762:H. sapiens mRNA for tafazzins protein /cds=(288,116; 38442_at Cluster Incl. U19718:Human microfibril-associated glycoprotein (MFAP2); 40205_g_at Cluster Incl. X71877:H. sapiens mRNA for chymotrypsin-like protease CT; 40565_at Cluster Incl. AI358867:qy24a09.x1 Homo sapiens cDNA, 3 end /clone=IMAG; 1799_at U64315 /FEATURE= /DEFINITION=HSU64315 Human DNA repair endonuclease subu; 480_at U56816 /FEATURE= /DEFINITION=HSU56816 Human kinase Myt1 (Myt1) mRNA, comp; 160037_at Z48482 /FEATURE=cds /DEFINITION=HSMMPM2 H. sapiens mRNA for membrane-ty.

Metagene 149; 36102_at Cluster Incl. AF038962:Homo sapiens voltage dependent anion channel pro; 696_at Homeotic Protein Hox5.4.

Metagene 150; 34578_at Cluster Incl. U34976:Human gamma-sarcoglycan mRNA, complete cds /cds=(1; 39603_at Cluster Incl. AB007926:Homo sapiens mRNA for KIAA0457 protein, partial; 36825_at Cluster Incl. X82200:H. sapiens Staf50 mRNA /cds=(122,1450) /gb=X82200 /; 35839_at Cluster Incl. D78130:Homo sapiens mRNA for squalene epoxidase, complete; 40954_at Cluster Incl. H94881:yu57f07.s1 Homo sapiens cDNA, 3 end /clone=IMAGE-.

Metagene 151; 699_s_at Mucin 1, Epithelial, Alt. Splice 6; 203_at M68891 /FEATURE= /DEFINITION=HUMGATA Human GATA-binding protein (GATA2) m.

Metagene 152; 39583_at Cluster Incl. AF030435:Homo sapiens glioma amplified on chromosome 1 pr; 36552_at Cluster Incl. AL080220:Homo sapiens mRNA; cDNA DKFZp586P0123 (from clon; 36567_at Cluster Incl. W26700:11h4 Homo sapiens cDNA /gb=W26700 /gi=1305802 /ug=; 39418_at Cluster Incl. AJ007398:Homo sapiens mRNA for PBK1 protein /cds=(5,1558); 35363_at Cluster Incl. AL080113:Homo sapiens mRNA; cDNA DKFZp586K2322 (from clon; 39512_s_at Cluster Incl. AA457029:aa38b10.s1 Homo sapiens cDNA, 3 end /clone=IM; 41539_at Cluster Incl. U77782:Human N-methyl-D-aspartate receptor 2C subunit pre; 33122_at Cluster Incl. N95393:zb68c09.s1 Homo sapiens cDNA, 3 end /clone=IMAGE-.

Metagene 153; 31622_f_at Cluster Incl. M10943:Human metallothionein-If gene (hMT-If) /cds=(0,1; 31623_f_at Cluster Incl. K01383:Human metallothionein-I-A gene, complete coding; 38164_at Cluster Incl. U57629:Human retinitis pigmentosa GTPase regulator (RPGR); 39594_f_at Cluster Incl. R93527:yq35f10.r1 Homo sapiens cDNA, 5 end /clone=IMAG; 41446_f_at Cluster Incl. H68340:yr82b10.s1 Homo sapiens cDNA, 3 end /clone=IMAG; 32079_at Cluster Incl. AB014539:Homo sapiens mRNA for KIAA0639 protein, partial; 39411_at Cluster Incl. AL080156:Homo sapiens mRNA; cDNA DKFZp434J214 (from clone; 40074_at Cluster Incl. X16396:Human mRNA for NAD-dependent methylene tetrahydrof; 33825_at Cluster Incl. X68733:H. sapiens gene for alpha1-antichymotrypsin, exon 1; 36130_f_at Cluster Incl. R92331:yq03h03.r1 Homo sapiens cDNA, 5 end /clone=IMAG; 36960_at Cluster Incl. U89278:Human polyhomeotic 2 homolog (HPH2) mRNA, complete; 38044_at Cluster Incl. AF035283:Homo sapiens clone 23916 mRNA sequence /cds=UNKN; 39120_at Cluster Incl. AA224832:nc33b06.s1 Homo sapiens cDNA /clone=IMAGE-100990; 40202_at Cluster Incl. D31716:Human mRNA for GC box bindig protein, complete cds; 926_at J03910 /FEATURE=mRNA /DEFINITION=HUMMT2A Human (clone 14VS) metallothione; 609_f_at M13485 /FEATURE=cds /DEFINITION=HUMMT1B2 Human metallothionein I-B gene.

Metagene 154; 39640_at Cluster Incl. AB016789:Homo sapiens mRNA for Glutamine-fructose-6-phosp; 37191_at Cluster Incl. D87463:Human mRNA for KIAA0273 gene, complete cds /cds=(4; 39040_at Cluster Incl. W28360:46f9 Homo sapiens cDNA /gb=W28360 /gi=1308371 /ug=; 39331_at Cluster Incl. X79535:H. sapiens mRNA for beta tubulin, clone nuk_{—}278 /cd; 35326_at Cluster Incl. AF004876:Homo sapiens 54TMp (54tm) mRNA, complete cds /cd; 40638_at Cluster Incl. X70944:H. sapiens mRNA for PTB-associated splicing factor; 41250_at Cluster Incl. U24169:Human JTV-1 (JTV-1) mRNA, complete cds /cds=(113,1; 1979_s_at X55504 /FEATURE=cds /DEFINITION=HSP120A H. sapiens mRNA for P120 antige; 296_at Tubulin, Beta; 297_g_at Tubulin, Beta.

Metagene 155; 39261_at Cluster Incl. L16896:Human zinc finger protein mRNA, complete cds /cds=; 32140_at Cluster Incl. Y08110:H. sapiens mRNA for mosaic protein LR11 /cds=(80,67; 34223_at Cluster Incl. M59818:Human granulocyte colony-stimulating factor recept; 1353_g_at U11870 /FEATURE=mRNA /DEFINITION=HSU11870 Human interleukin-8 receptor; 596_s_at M59820 /FEATURE=mRNA /DEFINITION=HUMGCSFR3 Human granulocyte colony-sti; 245_at M25280 /FEATURE= /DEFINITION=HUMLNHR Human lymph node homing receptor mRN.

Metagene 156; 31431_at Cluster Incl. U12255:Human IgG Fc receptor hFcRn mRNA, complete cds /cd; 31432_g_at Cluster Incl. U12255:Human IgG Fc receptor hFcRn mRNA, complete cds /; 31609_s_at Cluster Incl. L33799:Human procollagen C-proteinase enhancer protein; 35016_at Cluster Incl. M13560:Human Ia-associated invariant gamma-chain gene /cd; 35517_at Cluster Incl. M12807:Human T-cell surface glycoprotein T4 mRNA, complet; 33956_at Cluster Incl. AB018549:Homo sapiens MD-2 mRNA, complete cds /cds=(125,6; 35411_at Cluster Incl. AB018551:Homo sapiens ATPBL mRNA for coiled-coil protein,; 35965_at Cluster Incl. X51757:Human heat-shock protein HSP70B gene /cds=(0,1931); 36270_at Cluster Incl. U04343:Human CD86 antigen mRNA, complete cds /cds=(147,11; 36773_f_at Cluster Incl. M81141:Human MHC class II HLA-DQ-beta mRNA (DR7 DQw2),; 36789_f_at Cluster Incl. AF025534:Homo sapiens leucocyte immunoglobulin-like rec; 38894_g_at Cluster Incl. AL008637:Human DNA sequence from clone 833B7 on chromos; 38964_r_at Cluster Incl. U12707:Human Wiskott-Aldrich syndrome protein (WASP) mR; 39263_at Cluster Incl. M87434:Human 71 kDa 25 oligoadenylate synthetase (p69 2-; 39319_at Cluster Incl. U20158:Human 76 kDa tyrosine phosphoprotein SLP-76 mRNA,; 40296_at Cluster Incl. AL023653:Human DNA sequence from clone 753P9 on chromosom; 40365_at Cluster Incl. M63904:Human G-alpha 16 protein mRNA, complete cds /cds=(; 41609_at Cluster Incl. U15085:Human HLA-DMB mRNA, complete cds /cds=(233,1024) /; 31870_at Cluster Incl. X14046:Human mRNA for leukocyte antigen CD37 /cds=(63,908; 32046_at Cluster Incl. D10495:Homo sapiens mRNA for protein kinase C delta-type,; 32068_at Cluster Incl. U62027:Human anaphylatoxin C3a receptor (HNFAG09) mRNA, c; 32737_at Cluster Incl. M64595:Human small G protein (Gx) mRNA, 3 end /cds=(0,54; 34268_at Cluster Incl. X91809:H. sapiens m-RNA for GAIP protein /cds=(288,941) /gb; 34665_g_at Cluster Incl. X62573:H. sapiens RNA for Fc receptor, TC9 /cds=UNKNOWN; 35659_at Cluster Incl. U00672:Human interleukin-10 receptor mRNA, complete cds /; 36007_at Cluster Incl. AL050137:Homo sapiens mRNA; cDNA DKFZp586L151 (from clone; 36465_at Cluster Incl. U51127:Human interferon regulatory factor 5 (Humirf5) mRN; 36889_at Cluster Incl. M33195:Human Fc-epsilon-receptor gamma-chain mRNA, comple; 37200_at Cluster Incl. J04162:Human leukocyte IgG receptor (Fc-gamma-R) mRNA, co; 37918_at Cluster Incl. M15395:Human leukocyte adhesion protein (LFA-1/Mac-1/p150; 38287_at Cluster Incl. AA808961:nw16h03.s1 Homo sapiens cDNA /clone=IMAGE-124066; 38323_at Cluster Incl. AC005162:Homo sapiens BAC clone RG113D17 from 7p14-p15 /c; 38686_at Cluster Incl. X71490:H. sapiens mRNA for vacuolar proton ATPase, subunit; 39049_at Cluster Incl. AJ243937:Homo sapiens mRNA for G18.1a and G18.1b proteins; 39070_at Cluster Incl. U03057:Human actin bundling protein (HSN) mRNA, complete; 39345_at Cluster Incl. AI525834:PT1.3_{—}06_D01.r Homo sapiens cDNA, 5 end /clone_{—}; 39347_at Cluster Incl. X97074:H. sapiens mRNS for clathrin-associated protein /cd; 39428_at Cluster Incl. AF055581 Homo sapiens adaptor protein Lnk mRNA, complete; 40081_at Cluster Incl. L26232:Human phospholipid transfer protein mRNA, complete; 40505_at Cluster Incl. AA883502:am25h07.s1 Homo sapiens cDNA, 3 end /clone=IMAG; 41168_at Cluster Incl. AF029750:Homo sapiens tapasin (NGS-17) mRNA, complete cds; 41198_at Cluster Incl. AF055008:Homo sapiens clone 24720 epithelin 1 and 2 mRNA,; 41723_s_at Cluster Incl. M32578:Human MHC class II HLA-DR beta-1 mRNA (DR2.3), 5; 32157_at Cluster Incl. S57501:protein phosphatase type 1 catalytic subunit [huma; 33339_g_at Cluster Incl. M97936:Human transcription factor ISGF-3 mRNA sequence; 33871_s_at Cluster Incl. J02876:Human placental folate binding protein mRNA, com; 35807_at Cluster Incl. M21186:Human neutrophil cytochrome b light chain p22 phag; 36661_s_at Cluster Incl. X06882:Human gene for CD14 differentiation antigen /cds; 36959_at Cluster Incl. U49278:Homo sapiens UEV-1 (UBE2V) mRNA, partial cds /cds=; 36994_at Cluster Incl. M62762:Human vacuolar H+ ATPase proton channel subunit mR; 37310_at Cluster Incl. X02419:H. sapiens uPA gene /cds=(19,1414) /gb=X02419 /gi=; 37328_at Cluster Incl. X07743:Human mRNA for pleckstrin (P47) /cds=(60,1112) /gb; 37383_f_at Cluster Incl. X58536:Human mRNA for HLA class I locus C heavy chain /; 37402_at Cluster Incl. D26129:Human mRNA for ribonuclease A (RNase A), complete; 38745_at Cluster Incl. X76488:H. sapiens mRNA for lysosomal acid lipase /cds=(145; 38796_at Cluster Incl. X03084:Human mRNA for C1q B-chain of complement system /c; 39829_at Cluster Incl. AB016811:Homo sapiens mRNA for ADP ribosylation factor-li; 32616_at Cluster Incl. M16038:Human lyn mRNA encoding a tyrosine kinase /cds=(29; 33121_g_at Cluster Incl. AF045229:Homo sapiens regulator of G protein signaling; 1919_at XI 6316 /FEATURE=cds /DEFINITION=HSVAVPO Human mRNA for vav oncogene; 1810_s_at D10495 /FEATURE= /DEFINITION=HUMPKSCD Homo sapiens mRNA for protein ki; 1768_s_at X59932 /FEATURE=mRNA /DEFINITION=HSCSRCKIN Human mRNA for C-SRC-kinase; 1375_s_at M32304 /FEATURE= /DEFINITION=HUMMET Human metalloproteinase inhibitor; 1107_s_at M13755 /FEATURE=mRNA /DEFINITION=HUMIFN15K Human interferon-induced 17; 957_at Arrestin, Beta 2; 548_s_at S80267 /FEATURE= /DEFINITION=S80267 p72syk {G insertion nucleotide 92}; 478_g_at U51127 /FEATURE= /DEFINITION=HSU51127 Human interferon regulatory facto; 317_at D55696 /FEATURE= /DEFINITION=D55696 Homo sapiens mRNA for cysteine protea; 172_at U57650 /FEATURE= /DEFINITION=HSU57650 Human SH2-containing inositol 5-pho; 117_at X51757 /FEATURE=cds /DEFINITION=HSP70B Human heat-shock protein HSP70B g; 133_at X87212 /FEATURE=cds /DEFINITION=HSCATHCGE H. sapiens mRNA for cathepsin C; 160022_at X03663 /FEATURE=cds /DEFINITION=HSCFMS Human mRNA for c-fms proto-onco.

Metagene 157; 39988_at Cluster Incl. M74447:Human PSF-2 mRNA, complete cds /cds=(96,2207) /gb=; 33230_at Cluster Incl. AJ131186:Homo sapiens mRNA for nuclear matrix protein NMP; 34182_at Cluster Incl. U18932:Human heparan sulfate-N-deacetylase/N-sulfotransfe.

Metagene 158; 33634_at Cluster Incl. AF038007:Homo sapiens FIC1 mRNA, complete cds /cds=(0,375; 34919_at Cluster Incl. AJ223957:Homo sapiens mRNA for ARNO3 protein /cds=(25,122; 35001_at Cluster Incl. Z85986:Human DNA sequence from clone 108K11 on chromosome; 39254_at Cluster Incl. AL050011:Homo sapiens mRNA; cDNA DKFZp564G013 (from clone; 41089_at Cluster Incl. M83363:Human plasma membrane calcium-pumping ATPase (PMCA; 37979_at Cluster Incl. X80507:H. sapiens YAP65 mRNA /cds=((0,1364) /gb=X80507 /gi=; 37711_at Cluster Incl. S57212:hMEF2C=myocyte enhancer-binding factor 2 [human, s; 1671_s_at L35253 /FEATURE= /DEFINITION=HUMMAPKNS Human p38 mitogen activated pro; 1190_at Z48541 /FEATURE=cds /DEFINITION=HSPTPU2GN H. sapiens mRNA for protein tyr.

Metagene 159; 31587_at Cluster Incl. X96969:H. sapiens mRNA for urea transporter /cds=(273,1466; 32933_r_at Cluster Incl. AL050122:Homo sapiens mRNA; cDNA DKFZp586E121 (from clo; 34788_at Cluster Incl. AL049365:Homo sapiens mRNA; cDNA DKFZp586A0618 (from clon; 41497_at Cluster Incl. AI401296:tg92c03.x1 Homo sapiens cDNA, 3 end /clone=IMAG; 41507_at Cluster Incl. AI700753:we41b07.x1 Homo sapiens cDNA, 3 end /clone=IMAG.

Metagene 160; 34950_at Cluster Incl. AB018303:Homo sapiens mRNA for KIAA0760 protein, partial; 37447_at Cluster Incl. AF015257:Homo sapiens flow-induced endothelial G protein-; 41667_s_at Cluster Incl. AJ006068:Homo sapiens mRNA for dTDP-D-glucose 4,6-dehyd; 41715_at Cluster Incl. Y11312:H. sapiens mRNA for phosphoinositide 3-kinase /cds=; 31799_at Cluster Incl. AF070618:Homo sapiens clone 24627 mRNA sequence /cds=UNKN; 33294_at Cluster Incl. D29958:Human mRNA for KIAA0116 gene, partial cds /cds=(0,; 33796_at Cluster Incl. U73960:Human ADP-ribosylation factor-like protein 4 mRNA,; 40480_s_at Cluster Incl. M14333:Homo sapiens c-syn protooncogene mRNA, complete; 41220_at Cluster Incl. AB023208:Homo sapiens mRNA for KIAA0991 protein, complete; 36610_at Cluster Incl. D21852:Human mRNA for KIAA0029 gene, partial cds /cds=(38.

Metagene 161; 32998_at Cluster Incl. L19315:Human cholecystokinin A receptor mRNA, complete cd; 32968_s_at Cluster Incl. AL050253:H. sapiens mRNA similar to D29763 mouse mRNA fo; 41394_at Cluster Incl. AF038440:Homo sapiens phospholipase D2 (PLD2) mRNA, splic; 41689_at Cluster Incl. R16035:ya51h07.r2 Homo sapiens cDNA, 5 end /clone=IMAGE-; 40830_at Cluster Incl. AF012106:Homo sapiens DnaJ protein (HSPF2) mRNA, complete; 967_g_at X97795 /FEATURE=cds /DEFINITION=HSRAD54 H. sapiens mRNA homologous to S.

Metagene 162; 35414_s_at Cluster Incl. U77914:Human soluble protein Jagged mRNA, partial cds /; 41700_at Cluster Incl. M62424:Human thrombin receptor mRNA, complete cds /cds=(2; 32647_at Cluster Incl. AF060902:Homo sapiens vesicle soluble NSF attachment prot; 35643_at Cluster Incl. X76732:H. sapiens mRNA for NEFA protein /cds=(219,1481) /g; 39065_s_at Cluster Incl. D83077:Homo sapiens mRNA for TPRD, complete cds /cds=(1; 39351_at Cluster Incl. M84349:Human transmembrane protein (CD59) gene /cds=(18,4; 39397_at Cluster Incl. M64497:Human apolipoprotein AI regulatory protein (ARP-1); 41743_i_at Cluster Incl. AF061034:Homo sapiens FIP2 alternatively translated mRN; 32214_at Cluster Incl. AF003938:Homo sapiens thioredoxin-like protein mRNA, comp; 33402_at Cluster Incl. AL035081:H. sapiens mRNA similar to Xenopus laevis mRNA fo; 33429_at Cluster Incl. AL050225:Homo sapiens mRNA; cDNA DKFZp586M1523 (from clon; 33866_at Cluster Incl. X05276:Human mRNA for fibroblast tropomyosin TM30 (pl) /c; 33875_at Cluster Incl. AI547262:PN001_AH_H03.r Homo sapiens cDNA, 5 end /clone_{—}; 33930_at Cluster Incl. AB020724:Homo sapiens mRNA for KIAA0917 protein, partial; 36647_at Cluster Incl. AA526812:ni92a08.s1 Homo sapiens cDNA, 3 end /clone=IMAG; 38123_at Cluster Incl. D14878:Human mRNA for protein D123, complete cds /cds=(28; 39169_at Cluster Incl. AF054184:Homo sapiens Sec61 gamma mRNA, complete cds /cds; 39814_s_at Cluster Incl. AI052724:oz27a12.x1 Homo sapiens cDNA, 3 end /clone=IM; 40556_at Cluster Incl. D42073:Human mRNA for reticulocalbin, complete cds /cds=(; 2044_s_at M15400 /FEATURE=mRNA /DEFINITION=HUMRS Human retinoblastoma susceptib; 1884_s_at M15796 /FEATURE= /DEFINITION=HUMCYL Human cyclin protein gene, complet; 1798_at U41060 /FEATURE= /DEFINITION=HSU41060 Human breast cancer, estrogen regu; 1728_at L13689 /FEATURE=mRNA /DEFINITION=HUMBMI1X Human prot-oncogene (BMI-1) mR; 994_at X58288 /FEATURE=mRNA /DEFINITION=HSHRPTPU H. sapiens hR-PTPu gene for prot.

Metagene 163; 36706_at Cluster Incl. Y15057:Homo sapiens mRNA for STK9 protein /cds=(221,3313); 37237_at Cluster Incl. D38293:Homo sapiens mRNA for clathrin-like protein, compl; 37589_at Cluster Incl. AL109698:Homo sapiens mRNA full length insert cDNA clone; 37957_at Cluster Incl. AL031177:dJ889M15.3 (novel protein) /cds=(0,1186) /gb=AL0; 38296_at Cluster Incl. AL050196:Homo sapiens mRNA; cDNA DKFZp586D2223 (from clon; 35368_at Cluster Incl. AF046001:Homo sapiens zinc finger transcription factor (Z; 35743_at Cluster Incl. U79569:Human no arches (nar) mRNA, complete cds /cds=(36,; 1523_g_at U43408 /FEATURE= /DEFINITION=HSU43408 Human tyrosine kinase (Tnk1) mRN; 1187_at X84740 /FEATURE=mRNA /DEFINITION=HSDNALIG3 H. sapiens mRNA for DNA ligase.

Metagene 164; 34477_at Cluster Incl. AF000994:Homo sapiens ubiquitous TPR motif, Y isoform (UT; 35885_at Cluster Incl. AF000986:Homo sapiens Drosophila fat facets related Y pro; 37583_at Cluster Incl. U52191:Human SMCY (H-Y) mRNA, complete cds /cds=(275,4894; 38355_at Cluster Incl. AF000984:Homo sapiens dead box, Y isoform (DBY) mRNA, alt; 40097_at Cluster Incl. AF000987:Homo sapiens eIF-1A, Y isoform (EIF1AY) mRNA, co; 41214_at Cluster Incl. M58459:Human ribosomal protein (RPS4Y) isoform mRNA, comp.

Metagene 165; 34812_at Cluster Incl. W26099:22f11 Homo sapiens cDNA /gb=W26099 /gi=1306645 /ug; 34854_at Cluster Incl. AC004382:Homo sapiens Chromosome 16 BAC clone CIT987SK-A-; 32591_at Cluster Incl. AI494623:qz17b06.x1 Homo sapiens cDNA, 3 end /clone=IMAG; 1729_at L41690 /FEATURE= /DEFINITION=HUMTRADD Homo sapiens TNF receptor-1 associ; 1317_at X70040 /FEATURE=cds /DEFINITION=HSRON H. sapiens RON mRNA for tyrosine ki; 1254_at L36861 /FEATURE=expanded_cds /DEFINITION=HUMGCAPB Homo sapiens guanylate.

Metagene 166; 32331_at Cluster Incl. X60673:Human AK3 mRNA for adenylate kinase 3 /cds=UNKNOWN; 40322_at Cluster Incl. D12763:Homo sapiens mRNA for ST2 protein /cds=(46,1032) /; 33769_at Cluster Incl. AF087020:Homo sapiens protein zero related protein (PZR); 36490_s_at Cluster Incl. X15331:Human mRNA for phosphoribosylpyrophosphate synth; 37952_at Cluster Incl. M35999:Human platelet glycoprotein IIIa (GPIIIa) mRNA, co; 39002_at Cluster Incl. AJ010063:Homo sapiens telethonin gene /cds=(10,513) /gb=A; 41782_g_at Cluster Incl. U22815:Human LAR-interacting protein 1a mRNA, complete; 35305_at Cluster Incl. X95762:H. sapiens mRNA for aminopeptidase P-like /cds=(0,1; 36101_s_at Cluster Incl. M63978:Human vascular endothelial growth factor gene /c; 39810_at Cluster Incl. AC005253:Homo sapiens chromosome 19, cosmid R26445 /cds=(; 1735_g_at M60556 /FEATURE=mRNA#1 /DEFINITION=HUMTGFB3B Human transforming growth; 1454_at U68019 /FEATURE= /DEFINITION=HSU68019 Homo sapiens mad protein homolog (; 1318_at X74262 /FEATURE=cds /DEFINITION=HSRBAP48 H. sapiens RbAp48 mRNA encoding; 759_at D38145 /FEATURE= /DEFINITION=HUMPTGIS Human mRNA for prostacyclin synthas; 275_at L05147 /FEATURE= /DEFINITION=HUMDSPHS Human dual specificity phosphatase; 231_at M55153 /FEATURE= /DEFINITION=HUMTGASE Human transglutaminase (TGase) mRNA.

Metagene 167; 33613_at Cluster Incl. AA806239:oc21e02.s1 Homo sapiens cDNA /clone=IMAGE-134153; 35606_at Cluster Incl. D16583:Human gene for L-histidine decarboxylase, complete; 36749_at Cluster Incl. M73720:Human mast cell carboxypeptidase A (MC-CPA) gene /; 37121_at Cluster Incl. S69115:granulocyte colony-stimulating factor induced gene; 37454_at Cluster Incl. AJ001634:Homo sapiens mRNA for CC-chemokine MCP-4 /cds=(4; 37493_at Cluster Incl. H04668:yj49e08.r1 Homo sapiens cDNA, 5 end /clone=IMAGE-; 37823_at Cluster Incl. Y16645:Homo sapiens mRNA for monocyte chemotactic protein; 38131_at Cluster Incl. AF010316:Homo sapiens Pig12 (PIG12) mRNA, complete cds /c; 38222_at Cluster Incl. AF007893:Homo sapiens P2Y6 receptor pseudogene, complete; 40671_g_at Cluster Incl. AI148772:qc69h01.x1 Homo sapiens cDNA, 3 end /clone=IM; 41045_at Cluster Incl. U77643:Homo sapiens K12 protein precursor mRNA, complete; 41385_at Cluster Incl. AB023204:Homo sapiens mRNA for KIAA0987 protein, partial; 41433_at Cluster Incl. M73255:Human vascular cell adhesion molecule-1 (VCAM1) ge; 31895_at Cluster Incl. AB002803:Homo sapiens BACH1 mRNA, complete cds /cds=(118,; 31898_at Cluster Incl. D86967:Human mRNA for KIAA0212 gene, complete cds /cds=(5; 32034_at Cluster Incl. AF041259:Homo sapiens breast cancer putative transcriptio; 32666_at Cluster Incl. U19495:Human intercrine-alpha (hIRH) mRNA, complete cds /; 34663_at Cluster Incl. M28696:Human low-affinity IgG Fc receptor (beta-Fc-gamma-; 36070_at Cluster Incl. AL049389:Homo sapiens mRNA; cDNA DKFZp586O0118 (from clon; 36879_at Cluster Incl. M63193:Human platelet-derived endothelial cell growth fac; 37215_at Cluster Incl. AF046798:untitled /cds=(113,2656) /gb=AF046798 /gi=317040; 37603_at Cluster Incl. X52015:H. sapiens mRNA for interleukin-1 receptor antagoni; 38652_at Cluster Incl. AF070644:Homo sapiens clone 24742 mRNA sequence /cds=UNKN; 39043_at Cluster Incl. AF006084:Homo sapiens Arp2/3 protein complex subunit p41-; 39338_at Cluster Incl. AI201310:qf71b11.x1 Homo sapiens cDNA, 3 end /clone=IMAG; 39421_at Cluster Incl. D43969:Human AML1 mRNA for AML1c protein (alternatively s; 40520_g_at Cluster Incl. Y00638:Human mRNA for leukocyte common antigen (T200) /; 41184_s_at Cluster Incl. X87344:H. sapiens DMA, DMB, HLA-Z1, IPP2, LMP2, TAP1, LM; 32860_g_at Cluster Incl. M97935:Homo sapiens transcription factor ISGF-3 mRNA, c; 33452_at Cluster Incl. M15518:Human tissue-type plasminogen activator (t-PA) mRN; 33834_at Cluster Incl. L36033:Human pre-B cell stimulating factor homologue (SDF; 36986_at Cluster Incl. AL031295:Human DNA sequence from clone 886K2 on chromosom; 36988_at Cluster Incl. M80783:Human B12 protein mRNA, complete cds /cds=(153,110; 38369_at Cluster Incl. U70451:Human myleoid differentiation primary response pro; 38389_at Cluster Incl. X04371:Human 1.6 Kb mRNA for 2-5A synthetase induced by in; 40639_at Cluster Incl. AL021683:Homo sapiens cDNA homologous to Yeast SCO1 & SCO; 32614_at Cluster Incl. AB018278:Homo sapiens mRNA for KIAA0735 protein, complete; 1456_s_at M63838 /FEATURE= /DEFINITION=HUMIFI16A Human interferon-gamma induced; 1039_s_at U22431 /FEATURE= /DEFINITION=HSU22431 Human hypoxia-inducible factor 1; 999_at X59812 /FEATURE=cds /DEFINITION=HSVD3HYD H. sapiens CYP 27 mRNA for vitami; 943_at D43968 /FEATURE= /DEFINITION=HUMAML1BA Human AML1 mRNA for AML1b protein; 859_at U03688 /FEATURE= /DEFINITION=HSU03688 Human dioxin-inducible cytochrome P; 583_s_at M30257 /FEATURE= /DEFINITION=HUMCAM1V Human vascular cell adhesion mole; 570_at M83221 /FEATURE= /DEFINITION=HUMIRELA Homo sapiens I-Rel mRNA, complete c; 479_at U53446 /FEATURE= /DEFINITION=HSU53446 Human mitogen-responsive phosphopro; 393_s_at X90976 /FEATURE= /DEFINITION=HSRNAML11 H. sapiens mRNA for an acute mye1.

Metagene 168; 35553_at Cluster Incl. AF042181:Homo sapiens testis-specific Y-encoded-like prot.

Metagene 169; 31526_f_at Cluster Incl. X63547:H. sapiens mRNA for tre oncogene (clone 213) /cds; 33620_at Cluster Incl. X16667:Human HOX2G mRNA from the Hox2 locus /cds=(363,165; 34077_at Cluster Incl. X95876:H. sapiens mRNA for G-protein coupled receptor /cds; 34503_at Cluster Incl. AF007146:Homo sapiens clone 23686 and 23885 mRNA sequence; 36289_f_at Cluster Incl. U27333:Human alpha (1,3) fucosyltransferase (FUT6) mRNA; 32048_at Cluster Incl. AL049675:Human gene from PAC 886K2, chromosome 1 /cds=UNK; 34755_at Cluster Incl. AJ236876:Homo sapiens mRNA for poly(ADP-ribose) polymeras; 38350_f_at Cluster Incl. AF005392:Homo sapiens alpha tubulin (TUBA2) gene, parti; 2038_g_at M13995 /FEATURE=mRNA /DEFINITION=HUMBCL2B Human B-cell leukemia/lympho; 1652_at U77735 /FEATURE= /DEFINITION=HSU77735 Human pim-2 protooncogene homolog; 685_f_at K03460 /FEATURE=cds /DEFINITION=HUMTUBA2H Human alpha-tubulin isotype H.

Metagene 170; 37631_at Cluster Incl. U14391:Human myosin-IC mRNA, complete cds /cds=(375,3704); 40417_at Cluster Incl. D43950:Human mRNA for KIAA0098 gene, partial cds /cds=(0,; 32830_g_at Cluster Incl. X97544:H. sapiens mRNA for TIM17 preprotein translocase; 33820_g_at Cluster Incl. X13794:H. sapiens lactate dehydrogenase B gene exon 1 an; 35272_at Cluster Incl. AI541042:pec1.2-1.D12.r Homo sapiens cDNA, 5 end /clone_{—}; 38125_at Cluster Incl. M14083:Human beta-migrating plasminogen activator inhibit; 40637_at Cluster Incl. Y00371:Human hsc70 gene for 71 kd heat shock cognate prot; 1179_at Heat Shock Protein, 70 Kda; 1180_g_at Heat Shock Protein, 70 Kda; 672_at J03764 /FEATURE=cds /DEFINITION=HUMPAIA Human, plasminogen activator inhi; 115_at X14787 /FEATURE=cds /DEFINITION=HSTS Human mRNA for thrombospondin.

Metagene 171; 31488_s_at Cluster Incl. S81916:phosphoglycerate kinase {alternatively spliced}; 31728_at Cluster Incl. M29335:Human MHC class II DO-alpha mRNA, partial cds /cds; 31947_r_at Cluster Incl. X74143:H. sapiens HBF-2 mRNA for transcription factor /c; 32408_s_at Cluster Incl. AL022101:dJ845O24.4 (Heterogenous Nuclear Ribonucleopro; 35036_at Cluster Incl. U94333:Human C1q/MBL/SPA receptor C1qR(p) mRNA, complete; 34909_at Cluster Incl. AC004990:Homo sapiens PAC clone DJ1185I07 from 7q11.23-q2; 37434_at Cluster Incl. W28907:53e12 Homo sapiens cDNA /gb=W28907 /gi=1308855 /ug; 37770_at Cluster Incl. AF026445:Homo sapiens cofactor of initiator function (CIF; 38500_at Cluster Incl. AB002450:Homo sapiens mRNA from chromosome 5q21-22, clone; 38552_f_at Cluster Incl. AI984786:wr85c06.x1 Homo sapiens cDNA, 3 end /clone=IM; 39613_at Cluster Incl. X74837:H. sapiens HUMM9 mRNA /cds=(689,2566) /gb=X74837 /g; 36513_at Cluster Incl. U37283:Human microfibril-associated glycoprotein-2 MAGP-2; 39368_at Cluster Incl. AL031668:Human DNA sequence from clone 64K7 on chromosome; 32790_at Cluster Incl. D59253:Human mRNA for NCBP interacting protein 1, complet; 38085_at Cluster Incl. AI740522:wg16b07.x1 Homo sapiens cDNA, 3 end /clone=IMAG; 39883_at Cluster Incl. AF091078:Homo sapiens clone 559 unknown mRNA, complete se; 40941_at Cluster Incl. W27026:19d10 Homo sapiens cDNA /gb=W27026 /gi=1306398 /ug; 2074_at L34954 /FEATURE=cds /DEFINITION=HUMCONNNEX Human connexin 40 gene, compl; 1968_g_at X76079 /FEATURE=exons#1-4 /DEFINITION=HSPDGF H. sapiens mRNA for platel; 1773_at L00635 /FEATURE= /DEFINITION=HUMFPTB Human farnesyl-protein transferase; 1734_at M60556 /FEATURE=mRNA#1 /DEFINITION=HUMTGFB3B Human transforming growth f; 1457_at M64174 /FEATURE= /DEFINITION=HUMPTKJAK1 Human protein-tyrosine kinase (J; 1321_s_at U43916 /FEATURE= /DEFINITION=HSU43916 Human tumor-associated membrane; 1267_at M55284 /FEATURE= /DEFINITION=HUMPKCL Human protein kinase C-L (PRKCL) mR; 1236_s_at M89914 /FEATURE= /DEFINITION=HUMNF1B Human neurofibromin (NF1) gene, c; 1164_at Ubiquitin-Conjugating Enzyme Ubch5; 1074_at M28209 /FEATURE= /DEFINITION=HUMRAB1A Homo sapiens GTP-binding protein (; 865_at U08316 /FEATURE= /DEFINITION=HSU08316 Human insulin-stimulated protein ki; 262_at M21154 /FEATURE=mRNA /DEFINITION=HUMAMD Human S-adenosylmethionine decarb.

Metagene 172; 37781_at Cluster Incl. AB023138:Homo sapiens mRNA for KIAA0921 protein, partial; 40677_at Cluster Incl. AF054984:Homo sapiens clone 23709 mRNA sequence /cds=UNKN; 37998_at Cluster Incl. U09877:Human helicase-like protein (HLP) mRNA, complete c; 35801_at Cluster Incl. AF026816:Homo sapiens putative oncogene protein mRNA, par; 37355_at Cluster Incl. D38255:Homo sapiens mRNA for CAB1, complete cds /cds=(121; 443_at X17360 /FEATURE=mRNA /DEFINITION=HSHOX51 Human HOX 5.1 gene for HOX 5.1 p; 251_at L41816 /FEATURE=mRNA /DEFINITION=HUMCKI Homo sapiens cam kinase I mRNA, c.

Metagene 173; 31495_at Cluster Incl. D63789:Homo sapiens DNA for SCM-1beta precursor, complete; 32488_at Cluster Incl. X14420:Human mRNA for pro-alpha-1 type 3 collagen /cds=(1; 32305_at Cluster Incl. J03464:Human collagen alpha-2 type I mRNA, complete cds,; 35937_at Cluster Incl. U65416:Human MHC class I molecule (MICB) gene, complete c; 36758_at Cluster Incl. AF070578:Homo sapiens clone 24674 mRNA sequence /cds=UNKN; 31900_at Cluster Incl. U33429:human K+ channel beta 2 subunit mRNA, complete cds; 33243_at Cluster Incl. AF099935:Homo sapiens MDC-3.13 isoform 2 mRNA, complete c; 39400_at Cluster Incl. AB028978:Homo sapiens mRNA for KIAA1055 protein, partial; 39438_at Cluster Incl. AF039081:Homo sapiens Cre binding protein-like 2 mRNA, co; 40797_at Cluster Incl. AF009615:Homo sapiens ADAM10 (ADAM10) mRNA, complete cds; 32148_at Cluster Incl. AI701049:wc78b08.x1 Homo sapiens cDNA, 3 end /clone=IMAG; 33936_at Cluster Incl. D86181:Homo sapiens DNA for galactocerebrosidase /cds=(14; 35786_at Cluster Incl. AB007945:Homo sapiens mRNA for KIAA0476 protein, complete; 36607_at Cluster Incl. Z99716:bK250D10.5 (alpha-N-acetylgalactosaminidase) /cds=; 36976_at Cluster Incl. D21255:Human mRNA for OB-cadherin-2, complete cds /cds=(4; 38427_at Cluster Incl. L25286:Homo sapiens alpha-1 type XV collagen mRNA, comple; 38446_at Cluster Incl. X56199:Human XIST, coding sequence a mRNA (locus DXS399; 39517_at Cluster Incl. AF035313:Homo sapiens clone 23851 mRNA sequence /cds=UNKN; 2087_s_at D21254 /FEATURE= /DEFINITION=HUMOSF4A Human mRNA for OB-cadherin-1, co; 1877_g_at Guanine Nucleotide-Binding Protein Ral, Ras-Oncogene Related; 1700_at U82987 /FEATURE= /DEFINITION=HSU82987 Human Bcl-2 binding component 3 (b; 1589_s_at L42243 /FEATURE=cds#2 /DEFINITION=HUMIFNAM08 Homo sapiens (clone 51H8); 1136_at L16991 /FEATURE= /DEFINITION=HUMCDC8X Human thymidylate kinase (CDC8) mR; 402_s_at X69819 /FEATURE=cds /DEFINITION=HSICAM3RN H. sapiens ICAM-3 mRNA; 160027_s_at Y00285 /FEATURE=cds /DEFINITION=HSIGFIIR Human mRNA for insuline-lik.

Metagene 174; 38915_at Cluster Incl. AB011135:Homo sapiens mRNA for KIAA0563 protein, complete; 36496_at Cluster Incl. AF014398:Homo sapiens myo-inositol monophosphatase 2 mRNA; 36566_at Cluster Incl. AJ222967:Homo sapiens mRNA for cystinosin /cds=(339,1442); 36915_at Cluster Incl. AI810485:wb89b12.x1 Homo sapiens cDNA, 3 end /clone=IMAG; 38440_s_at Cluster Incl. AA015605:ze20c12.s1 Homo sapiens cDNA, 3 end /clone=IM; 33178_at Cluster Incl. AF003837:Homo sapiens Jagged1 (JAG1) mRNA, complete cds /; 760_at Y09216 /FEATURE= /DEFINITION=HSDYRK2 H. sapiens mRNA for protein kinase, D.

Metagene 175; 34549_g_at Cluster Incl. X55764:Human mRNA for cytochrome P-450 (11 Beta) /cds=(; 40651_s_at Cluster Incl. AF039523:untitled /cds=(0,1334) /gb=AF039523 /gi=282812; 40741_at Cluster Incl. M30185:Human cholesteryl ester transfer protein mRNA, com; 34706_at Cluster Incl. AB011090:Homo sapiens mRNA for KIAA0518 protein, partial; 37274_at Cluster Incl. AF018631:untitled /cds=(35,1666) /gb=AF018631 /gi=2674074; 37554_at Cluster Incl. U62801:Human protease M mRNA, complete cds /cds=(245,979); 33455_at Cluster Incl. X02747:Human mRNA for aldolase B /cds=(125,1219) /gb=X027; 38464_at Cluster Incl. X87237:H. sapiens mRNA for processing a-glucosidase 1 /cds; 40523_at Cluster Incl. AI371038:ta29f02.x1 Homo sapiens cDNA, 3 end /clone=IMAG; 40927_at Cluster Incl. W26324:28h9 Homo sapiens cDNA /gb=W26324 /gi=1307086 /ug=; 41279_f_at Cluster Incl. AF007134:Homo sapiens clone 23565 unknown mRNA, partial; 41588_at Cluster Incl. AA022949:ze72h02.s1 Homo sapiens cDNA, 3 end /clone=IMAG; 1908_at L16464 /FEATURE= /DEFINITION=HUMETSONC Human ETS oncogene (PEP1) mRNA, c.

Metagene 176; 32916_at Cluster Incl. X54134:Human HPTP epsilon mRNA for protein tyrosine phosp; 38138_at Cluster Incl. D38583:Human mRNA for calgizzarin, complete cds /cds=(120; 39670_at Cluster Incl. AL050034:Homo sapiens mRNA; cDNA DKFZp566G0224 (from clon; 39982_r_at Cluster Incl. D13265:Human mRNA for macrophage scavenger receptor typ; 34774_at Cluster Incl. U44772:Human palmitoyl protein thioesterase mRNA, complet; 36128_at Cluster Incl. L40397:Homo sapiens (clone S31i125) mRNA, 3 end of cds /; 37324_at Cluster Incl. X01060:Human mRNA for transferrin receptor /cds=(263,2545; 37689_s_at Cluster Incl. X68090:H. sapiens Fc-gamma-RIIA gene for IgG Fc receptor; 37741_at Cluster Incl. M77836:Human pyrroline 5-carboxylate reductase mRNA, comp; 1786_at U08023 /FEATURE= /DEFINITION=HSU08023 Human cellular proto-oncogene (c-m; 1140_at L25851 /FEATURE= /DEFINITION=HUMINAE Homo sapiens integrin alpha E precu; 642_s_at L76528 /FEATURE=expanded_cds /DEFINITION=HUMPS1A11 Homo sapiens preseni; 467_at U63717 /FEATURE= /DEFINITION=HSU63717 Homo sapiens osteoclast stimulating; 338_at AF005887 /FEATURE= /DEFINITION=AF005887 Homo sapiens ATF family member AT.

Metagene 177; 35055_at Cluster Incl. X53281:H. sapiens BTF3b mRNA /cds=(239,727) /gb=X53281 /gi; 32111_at Cluster Incl. AL050164:Homo sapiens mRNA; cDNA DKFZp586C1622 (from clon; 34216_at Cluster Incl. AA478904:zv20c05.r1 Homo sapiens cDNA, 5 end /clone=IMAG; 35635_at Cluster Incl. AL080202:Homo sapiens mRNA; cDNA DKFZp434F172 (from clone; 36515_at Cluster Incl. AJ238764:Homo sapiens mRNA for UDP-N-acetylglucosamine-2-; 40508_at Cluster Incl. AF025887:Homo sapiens glutathione S-transferase A4-4 (GST; 41136_s_at Cluster Incl. Y00264:Human mRNA for amyloid A4 precursor of Alzheimer; 41231_f_at Cluster Incl. X13546:Human HMG-17 gene for non-histone chromosomal pr; 32244_at Cluster Incl. AB018280:Homo sapiens mRNA for KIAA0737 protein, complete; 33827_at Cluster Incl. AL049783:Novel human gene mapping to chomosome 13 /cds=(1; 34340_at Cluster Incl. AA173896:zp03b02.s1 Homo sapiens cDNA, 3 end /clone=IMAG; 34366_g_at Cluster Incl. AF042386:Homo sapiens cyclophilin-33B (CYP-33) mRNA, co; 34860_g_at Cluster Incl. Z98046:dJ14O9.2 (Melanoma-Associated Antigen MAGE LIKE); 35294_at Cluster Incl. M25077:Human SS-A/Ro ribonucleoprotein autoantigen 60 kd; 36201_at Cluster Incl. D13315:Human mRNA for lactoyl glutathione lyase /cds=(87,; 39865_at Cluster Incl. AI890903:wm91f10.x1 Homo sapiens cDNA, 3 end /clone=IMAG; 40617_at Cluster Incl. AC004381:Homo sapiens Chromosome 16 BAC clone CIT987SK-44; 40642_at Cluster Incl. AI312646:qp77f01.x1 Homo sapiens cDNA, 3 end /clone=IMAG; 41837_at Cluster Incl. AA149431:zl26a08.s1 Homo sapiens cDNA, 3 end /clone=IMAG; 1640_at U17714 /FEATURE= /DEFINITION=HSU17714 Homo sapiens putative tumor suppre; 1577_at M23263 /FEATURE= /DEFINITION=HUMARB Human androgen receptor mRNA, comple; 490_g_at U63329 /FEATURE=cds /DEFINITION=HSU63329 Human mutY homolog (hMYH) gene; 178_f_at U38964 /FEATURE=cds /DEFINITION=HSU38964 Human PMS2 related (hPMSR2) ge.

Metagene 178; 33589_at Cluster Incl. AF052103:Homo sapiens clone 23726 mRNA sequence /cds=UNKN; 33984_at Cluster Incl. M16660:Human 90-kDa heat-shock protein gene, cDNA, comple; 34222_at Cluster Incl. AL035289:H. sapiens gene from PAC 1026E2, partial /cds=(1,; 33352_at Cluster Incl. X57985:H. sapiens genes for histones H2B.1 and H2A /cds=(4; 36203_at Cluster Incl. X16277:Human gene for ornithine decarboxylase ODC (EC 4.1; 38030_at Cluster Incl. AB002330:Human mRNA for KIAA0332 gene, partial cds /cds=(; 40252_g_at Cluster Incl. U00943:Human clone A9A2BRB2 (CAC)n/(GTG)n repeat-contai; 1850_at U07418 /FEATURE= /DEFINITION=HSHMLHI Human DNA mismatch repair (hmlh1) m; 1081_at M33764 /FEATURE=cds /DEFINITION=HUMSODB Human ornithine decarboxylase ge; 946_at D50663 /FEATURE= /DEFINITION=D50663 Human mRNA for TCTEL1 gene, complete.

Metagene 179; 37462_i_at Cluster Incl. L21990:Human spliceosomal protein (SAP 62) gene, comple; 38918_at Cluster Incl. AF083105:Homo sapiens HMG box factor SOX-13 mRNA, complet; 40362_at Cluster Incl. X61498:H. sapiens mRNA for NF-kB subunit /cds=(163,2964) /; 41018_at Cluster Incl. AL050015:Homo sapiens mRNA; cDNA DKFZp564O243 (from clone; 41413_at Cluster Incl. AF037339:Homo sapiens cleft lip and palate transmembrane; 31837_at Cluster Incl. U62317:Chromosome 22q13 BAC Clone CIT987SK-384D8 complete; 35154_at Cluster Incl. W68046:zd42a12.s1 Homo sapiens cDNA, 3 end /clone=IMAGE-; 35205_at Cluster Incl. AL050280:Homo sapiens mRNA; cDNA DKFZp586B0519 (from clon; 35244_at Cluster Incl. AB007929:Homo sapiens mRNA for KIAA0460 protein, partial; 35675_at Cluster Incl. AF037261:Homo sapiens SH3-containing adaptor molecule-1 m; 37931_at Cluster Incl. X05299:Human mRNA (˜95%) for major centromere autoantigen; 38310_at Cluster Incl. AB014591:Homo sapiens mRNA for KIAA0691 protein, complete; 38721_at Cluster Incl. W72733:zd77h11.r1 Homo sapiens cDNA, 5 end /clone=IMAGE-; 39711_at Cluster Incl. J03075:Human 80K-H protein (kinase C substrate) mRNA, com; 40093_at Cluster Incl. X83425:H. sapiens LU gene for Lutheran blood group glycopr; 40817_at Cluster Incl. M96824:Human nucleobindin precursor mRNA, complete cds /c; 41134_at Cluster Incl. AB023181:Homo sapiens mRNA for KIAA0964 protein, complete; 32254_at Cluster Incl. AL050223:Homo sapiens mRNA; cDNA DKFZp586L1323 (from clon; 32836_at Cluster Incl. U56417:Human lysophosphatidic acid acyltransferase-alpha; 33398_at Cluster Incl. AB014570:Homo sapiens mRNA for KIAA0670 protein, partial; 33425_at Cluster Incl. X97548:H. sapiens mRNA for TIF1beta zinc finger protein /c; 33841_at Cluster Incl. R48209:yj63d12.s1 Homo sapiens cDNA, 3 end /clone=IMAGE-; 33879_at Cluster Incl. U79528:Human SR31747 binding protein 1 mRNA, complete cds; 36152_at Cluster Incl. X79353:H. sapiens XAP-4 mRNA for GDP-dissociation inhibito; 36162_at Cluster Incl. X64364:H. sapiens mRNA for M6 antigen /cds=(57,866) /gb=X6; 36183_at Cluster Incl. X86779:H. sapiens mRNA for FAST kinase /cds=(21,1670) /gb=; 36958_at Cluster Incl. X95735:Homo sapiens mRNA for zyxin /cds=(71,1789) /gb=X95; 37307_at Cluster Incl. X04828:Human mRNA for G(i) protein alpha-subunit (adenyla; 37346_at Cluster Incl. M57567:Human ADP-ribosylation factor (hARF5) mRNA, comple; 38766_at Cluster Incl. AB002307:Human mRNA for KIAA0309 gene, partial cds /cds=(; 40235_at Cluster Incl. L13738:Human activated p21cdc42Hs kinase (ack) mRNA, comp; 40580_r_at Cluster Incl. M24398:Human parathymosin mRNA, complete cds /cds=(300,; 40998_at Cluster Incl. AF071309:Homo sapiens OPA-containing protein mRNA, comple; 32553_at Cluster Incl. M94046:Human zinc finger protein (MAZ) mRNA /cds=UNKNOWN; 1926_at U48801 /FEATURE= /DEFINITION=HSU48801 Human vascular endothelial growth; 1642_at U35113 /FEATURE= /DEFINITION=HSU35113 Human metastasis-associated mta1 m; 1487_at L38487 /FEATURE=mRNA /DEFINITION=HUMHERRA1 Human estrogen receptor-relat; 1158_s_at J04046 /FEATURE=mRNA /DEFINITION=HUMCAMA Human calmodulin mRNA, comple; 497_at U32680 /FEATURE= /DEFINITION=HSU32680 Human CLN3 mRNA, complete cds; 388_at X80907 /FEATURE= /DEFINITION=HSPHOSINK H. sapiens mRNA for p85 beta subuni; 371_at Z56281 /FEATURE=cds /DEFINITION=HSIRF3MR H. sapiens mRNA for interferon re; 175_s_at U33053 /FEATURE= /DEFINITION=HSU33053 Human lipid-activated protein kin.

Metagene 180; 33072_at Cluster Incl. AF041245:Homo sapiens orexin receptor-2 mRNA, complete cd; 37508_f_at Cluster Incl. AA675900:g02504r Homo sapiens cDNA, 5 end /clone=g0250; 37519_at Cluster Incl. M10058:Human asialoglycoprotein receptor H1 mRNA, complet; 41003_at Cluster Incl. U41816:Human C-1 mRNA, complete cds /cds=(11,403) /gb=U41; 41435_at Cluster Incl. AB014554:Homo sapiens mRNA for KIAA0654 protein, partial; 34655_at Cluster Incl. AI951832:wx38b09.x1 Homo sapiens cDNA, 3 end /clone=IMAG; 36165_at Cluster Incl. W51774:zc48b04.r1 Homo sapiens cDNA, 5 end /clone=IMAGE-; 39472_s_at Cluster Incl. W25985:17e6 Homo sapiens cDNA /gb=W25985 /gi=1306252 /u; 32505_at Cluster Incl. W28652:50f5 Homo sapiens cDNA /gb=W28652 /gi=1308663 /ug=; 1477_s_at M61853 /FEATURE=mRNA /DEFINITION=HUM2C18 Human cytochrome P4502C18 (CY; 554_at U03634 /FEATURE= /DEFINITION=HSU03634 Human P47 LBC oncogene mRNA, comple.

Metagene 181; 32478_f_at Cluster Incl. AL031133:dJ281H8.4 (Ubiquitin-Like protein SMT3 LIKE) /; 35018_at Cluster Incl. U61538:Human calcium-binding protein chp mRNA, complete c; 36379_at Cluster Incl. AF085808:Homo sapiens uroplakin III mRNA, complete cds /c; 32970_f_at Cluster Incl. U77327:Human Ki-1/57 intracellular antigen mRNA, partia; 32731_at Cluster Incl. AB018247:Homo sapiens mRNA for Fe65L2, complete cds /cds=; 32739_at Cluster Incl. AA001791:zh86c04.r1 Homo sapiens cDNA, 5 end /clone=IMAG; 33814_at Cluster Incl. AF005046:Homo sapiens serine/threonine kinase mRNA, compl; 37246_at Cluster Incl. AF070535:Homo sapiens clone 24432 mRNA sequence /cds=UNKN; 40084_at Cluster Incl. U03494:Human transcription factor LSF mRNA, complete cds; 35815_at Cluster Incl. AL049470:Homo sapiens mRNA; cDNA DKFZp586L012 (from clone; 36132_at Cluster Incl. S74728:antiquitin=26g turgor protein homolog [human, kidn; 39094_at Cluster Incl. AI991631:wr12h09.x1 Homo sapiens cDNA, 3 end /clone=IMAG; 40640_at Cluster Incl. U62317:Chromosome 22q13 BAC Clone CIT987SK-384D8 complete; 32528_at Cluster Incl. Z50853:H. sapiens mRNA for CLPP /cds=(19,852) /gb=Z50853 /.

Metagene 182; 31478_at Cluster Incl. M16653:Human pancreatic elastase IIB mRNA, complete cds /; 35505_at Cluster Incl. AI290660:qm12b10.x1 Homo sapiens cDNA, 3 end /clone=IMAG; 32878_f_at Cluster Incl. AA524802:nh33h11.s1 Homo sapiens cDNA /clone=IMAGE-9542; 32881_at Cluster Incl. U88834:Human clone 295, 5cM region surrounding hepatocyte; 34430_at Cluster Incl. U70732:Human glutamate pyruvate transaminase (GPT) gene,; 35443_at Cluster Incl. AF060543:Homo sapiens importin alpha 7 subunit mRNA, comp; 36710_at Cluster Incl. Z38026:H. sapiens mRNA for FALL-39 peptide antibiotic /cds; 31828_r_at Cluster Incl. AF027516:Homo sapiens trans-golgi network glycoprotein; 35175_f_at Cluster Incl. X70940:H. sapiens mRNA for elongation factor 1 alpha-2 /; 35703_at Cluster Incl. X06374:Human mRNA for platelet-derived growth factor PDGF; 40777_at Cluster Incl. X87838:H. sapiens mRNA for beta-catenin /cds=(214,2559) /g; 33449_at Cluster Incl. AF054185:Homo sapiens proteasome subunit HSPC mRNA, compl; 35768_at Cluster Incl. AB014561:Homo sapiens mRNA for KIAA0661 protein, complete; 35810_at Cluster Incl. AI525393:PT1.1_{—}07_A11.r Homo sapiens cDNA, 5 end /clone_{—}; 39185_at Cluster Incl. AF091083:Homo sapiens clone 628 unknown mRNA, complete se; 40540_at Cluster Incl. AI926989:wo68e11.x1 Homo sapiens cDNA, 3 end /clone=IMAG; 2062_at L19182 /FEATURE= /DEFINITION=HUMMAC25X Human MAC25 mRNA, complete cds; 2007_g_at U31601 /FEATURE= /DEFINITION=HSU31601 Human tyrosine protein kinase (J; 1492_f_at M33317 /FEATURE=mRNA /DEFINITION=HUMCYIIA4A Human cytochrome P450IIA4; 566_at M79462 /FEATURE= /DEFINITION=HUMPML1 Human PML-1 mRNA, complete CDS; 559_s_at S38742 /FEATURE= /DEFINITION=S38742 HOX11=HOX11 homeodomain {homeobox}; 469_at U66406 /FEATURE= /DEFINITION=HSU66406 Human putative EPH-related PTK rece; 254_at M11353 /FEATURE= /DEFINITION=HUMHISH3C Human H3.3 histone class C mRNA, c.

Metagene 183; 35130_at Cluster Incl. X15722:Human mRNA for glutathione reductase (EC 1.6.4.2); 36798_g_at Cluster Incl. J04168:Human leukosialin mRNA, complete cds /cds=(95,12; 37940_f_at Cluster Incl. AA806768:ob91d06.s1 Homo sapiens cDNA /clone=IMAGE-1338; 40051_at Cluster Incl. D31762:Human mRNA for KIAA0057 gene, complete cds /cds=(7.

Metagene 184; 31363_at Cluster Incl. W27607:35b12 Homo sapiens cDNA /gb=W27607 /gi=1307555 /ug; 31694_at Cluster Incl. X82877:H. sapiens Na+-D-glucose cotransport regulator gene; 34088_at Cluster Incl. AF043469:Homo sapiens neurexophilin 4 (NPH4) mRNA, partia; 34985_at Cluster Incl. AF035408:Homo sapiens cartilage intermediate layer protei; 41075_at Cluster Incl. AF087003:Homo sapiens fibrousheathin I mRNA, complete cds; 41654_at Cluster Incl. X02994:Human mRNA for adenosine deaminase (adenosine amin; 36085_at Cluster Incl. X63749:H. sapiens DNA for Rod transducin alpha-subunit /cd.

Metagene 185; 31372_at Cluster Incl. W28203:43g3 Homo sapiens cDNA /gb=W28203 /gi=1308169 /ug=; 31921_at Cluster Incl. U56421:Human olfactory receptor (OLF3) gene, complete cds; 31975_at Cluster Incl. X55503:H. sapiens pseudogene for metallothionein and AG/CT; 33622_at Cluster Incl. Z26308:H. sapiens isoform 1 gene for L-type calcium channe; 32271_at Cluster Incl. X16707:Human fra-1 mRNA /cds=(34,849) /gb=X16707 /gi=3146; 36703_at Cluster Incl. U86358:Human chemokine (TECK) mRNA, complete cds /cds=(0,; 39657_at Cluster Incl. X07695:Human mRNA for cytokeratin 4 C-terminal region /cd; 40300_g_at Cluster Incl. AF091890:Homo sapiens G-protein coupled receptor RE2 nmR; 35685_at Cluster Incl. Z14000:H. sapiens RING1 gene /cds=(75,1208) /gb=Z14000 /gi; 36929_at Cluster Incl. U17760:Human laminin S B3 chain (LAMB3) gene /cds=(399,39; 38624_at Cluster Incl. AF054506:Homo sapiens erythroid K—Cl cotransporter splici; 37299_at Cluster Incl. J04501:Human muscle glycogen synthase mRNA, complete cds; 38377_at Cluster Incl. U47742:Human monocytic leukaemia zinc finger protein (MOZ; 41838_at Cluster Incl. X99270:H. sapiens Xq28, 2000 bp sequence contg. ORF /cds=(5; 731_f_at Mucin 3, Intestinal; 331_at Microtubule-Associated Protein Tau, Alt. Splice 5, Exon 4a.

Metagene 186; 31738_at Cluster Incl. W28912:53f11 Homo sapiens cDNA /gb=W28912 /gi=1308860 /ug; 33650_at Cluster Incl. W25911:14h11 Homo sapiens cDNA /gb=W25911 /gi=1306034 /ug; 33668_at Cluster Incl. AF037643:Homo sapiens 60S ribosomal protein L12 (RPL12) p; 34532_at Cluster Incl. AF035318:Homo sapiens clone 23705 mRNA sequence /cds=UNKN; 39325_at Cluster Incl. U81523:Human endometrial bleeding associated factor mRNA,; 39659_at Cluster Incl. L37936:Human nuclear-encoded mitochondrial elongation fac; 40014_at Cluster Incl. AF055020:Homo sapiens clone 24722 unknown mRNA, partial c; 40324_r_at Cluster Incl. AF070585:Homo sapiens clone 24675 mRNA sequence /cds=UN; 41418_at Cluster Incl. AB020628:Homo sapiens mRNA for KIAA0821 protein, complete; 33782_r_at Cluster Incl. AA587372:nn82f03.s1 Homo sapiens cDNA, 3 end /clone=IM; 34730_g_at Cluster Incl. AB029037:Homo sapiens mRNA for KIAA1114 protein, comple; 35138_at Cluster Incl. Y11997:H. sapiens mRNA for A-kinase anchoring protein AKAP; 35727_at Cluster Incl. AI249721:qj64d06.x1 Homo sapiens cDNA, 3 end /clone-IMAG; 36531_r_at Cluster Incl. AC005757:Homo sapiens chromosome 19, cosmid R32611 /cds; 36846_s_at Cluster Incl. AA121509:zk88c10.s1 Homo sapiens cDNA, 3 end /clone=IM; 37585_at Cluster Incl. X13482:Human mRNA for U2 snRNP-specific A protein /cds=(5; 38265_at Cluster Incl. AI538172:ti75f08.x1 Homo sapiens cDNA, 3 end /clone=IMAG; 38311_at Cluster Incl. AF055012:Homo sapiens clone 24615 mRNA sequence /cds=UNKN; 39749_at Cluster Incl. U51007:Human 26S protease subunit S5a mRNA, complete cds; 40110_at Cluster Incl. U49283:Human NAD+-specific isocitrate dehydrogenase beta; 40869_at Cluster Incl. AF069517:Homo sapiens RNA binding protein DEF-3 mRNA, com; 41205_at Cluster Incl. U84404:Human E6-associated protein E6-AP/ubiquitin-protei; 32774_at Cluster Incl. AI541050:pec1.2-1.E08.r Homo sapiens cDNA, 5 end /clone_{—}; 34345_at Cluster Incl. AF026031:Homo sapiens putative mitochondrial outer membra; 34882_at Cluster Incl. Y12065:Homo sapiens mRNA for nucleolar protein hNop56 /cd; 35736_at Cluster Incl. AL050091:Homo sapiens mRNA; cDNA DKFZp586F1918 (from clon; 36145_at Cluster Incl. U51586:Human siah binding protein 1 (SiahBP1) mRNA, parti; 36602_at Cluster Incl. D21064:Human mRNA for KIAA0123 gene, partial cds /cds=(0,; 37041_at Cluster Incl. AB023160:Homo sapiens mRNA for KIAA0943 protein, partial; 38053_-sat Cluster Incl. AF015767:Homo sapiens brain and reproductive organ-expr; 38060_at Cluster Incl. AI541336:pec1.2-7.A07.r Homo sapiens cDNA, 5 end /clone_{—}; 40210_at Cluster Incl. X75593:H. sapiens mRNA for rab 13 /cds=(139,750) /gb=X7559; 40926_at Cluster Incl. U36341:Human Xq28 cosmid, creatine transporter (SLC6A8) g; 41600_at Cluster Incl. U59435:Human cell cycle protein p38-2G4 homolog (hG4-1) m; 1357_at U20657 /FEATURE= /DEFINITION=HSU20657 Human ubiquitin protease (Unph) pr; 1308_g_at D14533 /FEATURE= /DEFINITION=HUMXPAC Human mRNA for XPAC protein; 1009_at U51004 /FEATURE= /DEFINITION=HSU51004 Homo sapiens protein kinase C inhi; 662_at L13848 /FEATURE= /DEFINITION=HUMRNAHELA Human RNA helicase A mRNA, comple; 571_at M86667 /FEATURE= /DEFINITION=HUMNAP H. sapiens NAP (nucleosome assembly pr; 472_at U48730 /FEATURE= /DEFINITION=HSU48730 Homo sapiens transcription factor S; 229_at M37197 /FEATURE=mRNA /DEFINITION=HUMCBF Human CCAAT-box-binding factor (C.

Metagene 187; 32303_at Cluster Incl. L16464:Human ETS oncogene (PEP1) mRNA, complete cds /cds=; 36761_at Cluster Incl. AL079276:Homo sapiens mRNA full length insert cDNA clone; 40688_at Cluster Incl. AJ223280:Homo sapiens mRNA for 36 kDa phosphothyrosine pr; 32225_at Cluster Incl. X04297:Human mRNA for Na,K-ATPase alpha-subunit /cds=(318; 39519_at Cluster Incl. AB014592:Homo sapiens mRNA for KIAA0692 protein, partial; 1650_g_at U61836 /FEATURE=mRNA /DEFINITION=HSU61836 Human putative cyclin G1 int.

Metagene 188; 36439_at Cluster Incl. AL080134:Homo sapiens mRNA; cDNA DKFZp434G043 (from clone; 39658_at Cluster Incl. AB007901:Homo sapiens KIAA0441 mRNA, complete cds /cds=(1; 1096_g_at M28170 /FEATURE= /DEFINITION=HUMCSPC Human cell surface protein CD19 (.

Metagene 189; 38863_at Cluster Incl. L07540:Human replication factor C, 36-kDa subunit mRNA, c; 41852_at Cluster Incl. U22377:Human Zn-15 related zinc finger protein (rlf) mRNA; 32743_at Cluster Incl. AB007922:Homo sapiens mRNA for KIAA0453 protein, partial; 951_at D88378 /FEATURE= /DEFINITION=D88378 Homo sapiens mRNA for proteasome inhi; 205_g_at M74297 /FEATURE= /DEFINITION=HUMHOX14 Human homeobox 1.4 protein mRNA,.

Metagene 190; 36307_at Cluster Incl. D87468:Human mRNA for KIAA0278 gene, partial cds /cds=(0,; 37128_at Cluster Incl. AB005216:Homo sapiens mRNA for Nck, Ash and phospholipase; 37473_at Cluster Incl. AF061812:Homo sapiens keratin 16 (KRT16A) mRNA, complete; 37858_at Cluster Incl. AF057036:Homo sapiens acetylcholinesterase collagen-like; 40302_at Cluster Incl. AL031186:bK984G1.1 (PUTATIVE C-terminal end of a novel pr; 32716_at Cluster Incl. X62535:H. sapiens mRNA for diacylglycerol kinase /cds=(103; 33758_f_at Cluster Incl. U25988:Human pregnancy-specific glycoprotein 13 (PSG13); 36621_at Cluster Incl. M16961:Human alpha-2-HS-glycoprotein alpha and beta chain; 37408_at Cluster Incl. AB014609:Homo sapiens mRNA for KIAA0709 protein, complete; 39821_s_at Cluster Incl. N95168:zb55f11.s1 Homo sapiens cDNA, 3 end /clone=IMAG; 40904_at Cluster Incl. AI857673:wk96c02.x1 Homo sapiens cDNA, 3 end /clone=IMAG; 40960_at Cluster Incl. D29805:Human mRNA for beta-1,4-galactosyltransferase, com.

Metagene 191; 31668_f_at Cluster Incl. W28193:43d12 Homo sapiens cDNA /gb=W28193 /gi=1308141 /; 38226_at Cluster Incl. W27152:23h11 Homo sapiens cDNA /gb=W27152 /gi=1306731 /ug; 37045_at Cluster Incl. D87443:Human mRNA for KIAA0254 gene, complete cds /cds=(5; 38438_at Cluster Incl. M58603:Human nuclear factor kappa-B DNA binding subunit (; 39139_at Cluster Incl. AI357653:qy15c11.x1 Homo sapiens cDNA, 3 end /clone=IMAG.

Metagene 192; 32420_at Cluster Incl. U18549:Human GPR6 G protein-coupled receptor gene, comple; 35923_at Cluster Incl. D13305:Human mRNA for brain cholecystokinin receptor /cds; 36252_at Cluster Incl. U43030:Human cardiotrophin-1 (CTF1) mRNA, complete cds /c; 39629_at Cluster Incl. U03090:Homo sapiens Ca2+-dependent phospholipase A2 mRNA,; 39333_at Cluster Incl. M26576:Human alpha-1 collagen type IV gene /cds=(0,5009); 32162_r_at Cluster Incl. AI817548:wk24e08.x1 Homo sapiens cDNA, 3 end /clone=IM; 41325_at Cluster Incl. AF006823:Homo sapiens TWIK-related acid-sensitive K+ chan; 33201_at Cluster Incl. AW007029:ws49c09.x1 Homo sapiens cDNA, 3 end /clone=IMAG; 2047_s_at M23410 /FEATURE= /DEFINITION=HUMPLAKO Human plakoglobin (PLAK) mRNA, c.

Metagene 193; 32444_at Cluster Incl. X69392:H. sapiens mRNA for ribosomal protein L26 /cds=(6,4; 32484_at Cluster Incl. U94888:Homo sapiens CC-chemokine receptor 10 (CCR10) mRNA; 32492_g_at Cluster Incl. U25441:Human dopamine D3 receptor (DRD3) gene, complete; 35590_s_at Cluster Incl. X81832:H. sapiens mRNA for glucose-dependant insulinotro; 37517_at Cluster Incl. AB028962:Homo sapiens mRNA for KIAA1039 protein, partial; 38507_at Cluster Incl. X16867:Human mRNA for cytochrome P-450IID (clone pMP34) /; 31815_r_at Cluster Incl. AB009462:Homo sapiens hLRp105 mRNA for LDL receptor rel; 32646_at Cluster Incl. AB007918:Homo sapiens mRNA for KIAA0449 protein, partial; 33268_at Cluster Incl. L25270:Human XE169 mRNA, complete cds /cds=(531,5213) /gb; 33768_at Cluster Incl. L19267:Homo sapiens 59 protein mRNA, 3 end /cds=(0,1661); 36084_at Cluster Incl. D38548:Human mRNA for KIAA0076 gene, complete cds /cds=(8; 33836_at Cluster Incl. AC002045:Human Chromosome 16 BAC clone CIT987SK-A-589H1 /; 37404_at Cluster Incl. AF030152:Homo sapiens regulator of G protein signaling 12; 38019_at Cluster Incl. L37043:Homo sapiens casein kinase I epsilon mRNA, complet; 38055_at Cluster Incl. AI683748:tw53e07.x1 Homo sapiens cDNA, 3 end /clone=IMAG; 39845_at Cluster Incl. AF020760:Homo sapiens serine protease (Omi) mRNA, complet; 33135_at Cluster Incl. U17566:Human 65 kDa hydrophobic protein mRNA, complete cd; 1627_at Tyrosine Kinase; 1008_f_at U50648 /FEATURE=mRNA /DEFINITION=HSIIPKR17 Human interferon-inducible; 678_at J04948 /FEATURE=mRNA /DEFINITION=HUMALPIGC Human alkaline phosphatase (AL; 454_at U66617 /FEATURE= /DEFINITION=HSU66617 Human SWI/SNF complex 60 KDa subuni.

Metagene 194; 36468_at Cluster Incl. U84551:Human dystrobrevin (DTN) gene /cds=(1,1713) /gb=U8; 40476_s_at Cluster Incl. U58198:Human interleukin enhancer binding factor 3 mRNA; 36592_at Cluster Incl. S85655:prohibitin [human, mRNA, 1043 nt]/cds=(50,868) /g; 1874_at D21090 /FEATURE= /DEFINITION=HUMHHR23B Human mRNA for XP-C repair comple; 491_at U46116 /FEATURE=mRNA /DEFINITION=HSPTPRG28 Human receptor tyrosine phosph; 267_at L34075 /FEATURE= /DEFINITION=HUMFRAPX Human FKBP-rapamycin associated pro; 123_at X75756 /FEATURE=cds /DEFINITION=HSPKCMU H. sapiens mRNA for protein kinase.

Metagene 195; 33466_at Cluster Incl. AF038182:Homo sapiens clone 23860 mRNA sequence /cds=UNKN; 35733_at Cluster Incl. AF006082:Homo sapiens actin-related protein Arp2 (ARP2) m; 1820_g_at Guanine Nucleotide-Binding Protein Rap2, Ras-Oncogene Related; 1284_at Serine/Threonine Kinase; 1253_at L33801 /FEATURE= /DEFINITION=HUMGLSYKIN Human protein kinase mRNA, compl.

Metagene 196; 32893_s_at Cluster Incl. M30474:Human kidney gamma-glutamyl transpeptidase type; 37463_r_at Cluster Incl. L21990:Human spliceosomal protein (SAP 62) gene, comple; 41035_at Cluster Incl. AB018318:Homo sapiens mRNA for KIAA0775 protein, complete; 37970_at Cluster Incl. AB028989:Homo sapiens mRNA for KIAA1066 protein, partial; 40472_at Cluster Incl. AF007155:Homo sapiens clone 23763 unknown mRNA, partial c; 38759_at Cluster Incl. U97502:Homo sapiens butyrophilin (BT3.3) gene /cds=(73,22; 38799_at Cluster Incl. AF068706:Homo sapiens gamma2-adaptin (G2AD) mRNA, complet; 1341_at X52056 /FEATURE=cds /DEFINITION=HSSPI1 Human mRNA for spi-1 proto-oncoge; 335_r_at Spliceosomal Protein Sap 62.

Metagene 197; 31724_at Cluster Incl. L38518:Homo sapiens sonic hedgehog protein (SHH) mRNA, co; 32990_at Cluster Incl. S75989:gamma-aminobutyric acid transporter type 3 [human,; 33580_r_at Cluster Incl. Z97630:Human DNA sequence from clone 466N1 on chromosom; 32379_f_at Cluster Incl. W28740:51a5 Homo sapiens cDNA /gb=W28740 /gi=1308688 /u; 35437_at Cluster Incl. M88461:Human neuropeptide Y peptide YY receptor mRNA, com; 40319_at Cluster Incl. H05552:yl75c01.r1 Homo sapiens cDNA, 5 end /clone=IMAGE-; 31893_at Cluster Incl. L13687:Homo sapiens ADP-ribosylation factor-like protein; 34224_at Cluster Incl. AC004770:Homo sapiens chromosome 11, BAC CIT-HSP-311e8 (B; 35666_at Cluster Incl. U38276:Human semaphorin III family homolog mRNA, complete; 37553_at Cluster Incl. D50863:Human mRNA for TESK1, complete cds /cds=(272,2152); 37894_at Cluster Incl. U83410:Human CUL-2 (cul-2) mRNA, complete cds /cds=(146,2; 37982_at Cluster Incl. Z25821:H. sapiens gene for mitochondrial dodecenoyl-CoA de; 39077_at Cluster Incl. U41843:Human Dr1-associated corepressor (DRAP1) mRNA, com; 40778_at Cluster Incl. AF035555:Homo sapiens short chain L-3-hydroxyacyl-CoA deh; 33347_at Cluster Incl. AA883868:am26e11.s1 Homo sapiens cDNA, 3 end /clone=IMAG; 34347_at Cluster Incl. AL049955:Homo sapiens mRNA; cDNA DKFZp564J0123 (from clon; 34351_at Cluster Incl. AL022394:dJ511B24.2 (1-Phosphatidylinositol-4,5-Bisphosph; 34358_at Cluster Incl. Z49254:H. sapiens L23-related mRNA /cds=(54,515) /gb=Z4925; 34784_at Cluster Incl. Z83844:Human DNA sequence from clone 37E16 on chromosome; 35773_i_at Cluster Incl. AA527880:nh86h10.s1 Homo sapiens cDNA, 3 end /clone=IM; 35774_r_at Cluster Incl. AA527880:nh86h10.s1 Homo sapiens cDNA, 3 end /clone=IM; 35813_at Cluster Incl. AA192359:zp91c12.s1 Homo sapiens cDNA, 3 end /clone=IMAG; 35819_at Cluster Incl. X06994:Human mRNA for cytochrome c1 /cds=(8,985) /gb=X069; 36936_at Cluster Incl. U58766:Human FX protein mRNA, complete cds /cds=(74,1039); 38397_at Cluster Incl. U09196:Human 1.1 kb mRNA upregulated in retinoic acid tre; 38451_at Cluster Incl. T58471:yb61c11.r1 Homo sapiens cDNA, 5 end /clone=IMAGE-; 39499_s_at Cluster Incl. W25794:13e8 Homo sapiens cDNA /gb=W2579⁴ /gi=1305935 /u; 40206_at Cluster Incl. AI767675:wh38d11.x1 Homo sapiens cDNA, 3 end /clone=IMAG; 41306_at Cluster Incl. AA004795:zh96a06.s1 Homo sapiens cDNA, 3 end /clone=IMAG; 32536_at Cluster Incl. Z37986:H. sapiens mRNA for phenylalkylamine binding protei; 32564_at Cluster Incl. AA083129:zn31a06.s1 Homo sapiens cDNA, 3 end /clone=IMAG; 1551_g_at U19796 /FEATURE= /DEFINITION=HSU19796 Human melanoma antigen p15 mRNA,; 1112_g_at M22092 /FEATURE=cds /DEFINITION=HUMNCAM2 Human neural cell adhesion mo; 592_at M34079 /FEATURE= /DEFINITION=HUMTBP1 Human immunodeficiency virus tat tra.

Metagene 198; 34422_r_at Cluster Incl. U82818:Homo sapiens UCP3S mRNA, complete cds /cds=(153,; 38546_at Cluster Incl. AB006537:Homo sapiens mRNA for interleukin 1 receptor acc; 39238_at Cluster Incl. AF040705:Homo sapiens putative tumor suppressor protein u; 41663_at Cluster Incl. AF038202:Homo sapiens clone 23570 mRNA sequence /cds=UNKN; 41686_s_at Cluster Incl. AL042668:DKFZp434O1721_r1 Homo sapiens cDNA, 5 end /cl; 31842_at Cluster Incl. AF038195:Homo sapiens clone 23661 unknown protein mRNA, c; 32115_r_at Cluster Incl. X68486:H. sapiens mRNA for A2a adenosine receptor /cds=(; 38324_at Cluster Incl. AD000684:Homo sapiens DNA from chromosome 19-cosmid R3087; 32775_r_at Cluster Incl. AB006746:Homo sapiens hMmTRA1b mRNA, complete cds /cds=; 38792_at Cluster Incl. AD001528:Homo sapiens spermidine aminopropyltransferase m; 40976_at Cluster Incl. AF052432:Homo sapiens katanin p80 subunit mRNA, complete; 32535_at Cluster Incl. X63556:H. sapiens mRNA for fibrillin /cds=(0,9010) /gb=X63; 32615_at Cluster Incl. J05032:Human aspartyl-tRNA synthetase alpha-2 subunit mRN; 1775_at L24559 /FEATURE= /DEFINITION=HUMDNSPOLA Homo sapiens DNA polymerase alph; 1419_g_at D29675 /FEATURE=exon /DEFINITION=HUMNOSB Human inducible nitric oxide; 1055_g_at M87339 /FEATURE= /DEFINITION=HUMACT1A Human replication factor C, 37-k.

Metagene 199; 34991_at Cluster Incl. U58331:Human placental delta sarcoglycan mRNA, complete c; 36799_at Cluster Incl. L37882:Human frizzled gene product mRNA, complete cds /cd; 39251_at Cluster Incl. M74091:Human cyclin mRNA /cds=UNKNOWN /gb=M74091 /gi=1806; 37906_at Cluster Incl. Z37976:H. sapiens mRNA for latent transforming growth fact; 39764_at Cluster Incl. Z22534:H. sapiens ALK-2 mRNA /cds=(103,1632) /gb=Z22534 /g; 34354_at Cluster Incl. M80634:Human keratinocyte growth factor receptor mRNA, co; 37743_at Cluster Incl. U60060:Human FEZ1 mRNA, complete cds /cds=(99,1277) /gb=U; 38087_s_at Cluster Incl. W72186:zd69b10.s1 Homo sapiens cDNA, 3 end /clone=IMAG; 628_at L37882 /FEATURE= /DEFINITION=HUMFRIZ Human frizzled gene product mRNA, co; 320_at D83703 /FEATURE= /DEFINITION=D83703 Homo sapiens mRNA for peroxisome asse.

Metagene 200; 34164_at Cluster Incl. R42599:yg02e11.s1 Homo sapiens cDNA, 3 end /clone=IMAGE-; 34425_at Cluster Incl. AF031469:Homo sapiens MHC class I related protein 1 isofo; 38538_at Cluster Incl. AB014602:Homo sapiens mRNA for KIAA0702 protein, complete; 39601_at Cluster Incl. AF061836:Homo sapiens putative tumor suppressor protein (; 39609_at Cluster Incl. U80457:Human transcription factor SIM2 short form mRNA, c; 33323_r_at Cluster Incl. X57348:H. sapiens mRNA (clone 9112) /cds=(165,911) /gb=X; 33812_at Cluster Incl. AL049415:Homo sapiens mRNA; cDNA DKFZp586N2119 (from clon; 37572_at Cluster Incl. AW043690:wy80b07.x1 Homo sapiens cDNA, 3 end /clone=IMAG; 37969_at Cluster Incl. M59979:Human prostaglandin endoperoxide synthase mRNA, co; 39789_at Cluster Incl. U96094:Human sarcolipin (SLN) mRNA, complete cds /cds=(16; 34779_at Cluster Incl. R90942:yp92b03.r1 Homo sapiens cDNA, 5 end /clone=IMAGE-; 39459_at Cluster Incl. W28765:51d2 Homo sapiens cDNA /gb=W28765 /gi=1308713 /ug=; 39482_at Cluster Incl. W26787:15d8 Homo sapiens cDNA /gb=W26787 /gi=1306078 /ug=; 40271_at Cluster Incl. D42085:Human mRNA for KIAA0095 gene, complete cds /cds=(6; 40981_at Cluster Incl. U00930:Human clone C4E 1.63 (CAC)n/(GTG)n repeat-containi; 1065_at U02687 /FEATURE= /DEFINITION=HSU02687 Human growth factor receptor tyros; 348_at D14678 /FEATURE= /DEFINITION=HUMMHCB Human mRNA for kinesin-related prote.

Metagene 201; 31672_g_at Cluster Incl. D82351:Human retropseudogene MSSP-1 DNA, complete cds /; 40015_at Cluster Incl. AB002301:Human mRNA for KIAA0303 gene, partial cds /cds=(; 34705_at Cluster Incl. AJ224335:Homo sapien mRNA for putative secretory protein,; 35694_at Cluster Incl. AB014587:Homo sapiens mRNA for KIAA0687 protein, partial; 40061_at Cluster Incl. D83784:Human mRNA for KIAA0198 gene, partial cds /cds=(0,; 37674_at Cluster Incl. Y00451:Human mRNA for 5-aminolevulinate synthase /cds=(83; 39528_at Cluster Incl. L24564:Human Rad mRNA, complete cds /cds=(123,1049) /gb=L; 1776_at L24564 /FEATURE= /DEFINITION=HUMRAD Human Rad mRNA, complete cds.

Metagene 202; 31715_at Cluster Incl. U93720:Homo sapiens TEX28 mRNA, complete cds /cds=(144,13; 33555_at Cluster Incl. AF041261:Homo sapiens immunoglobulin-like transcript 7 mR; 37867_at Cluster Incl. Z48051:H. sapiens gene for myelin oligodendrocyte glycopro; 41247_at Cluster Incl. H12054:ym11b12.r1 Homo sapiens cDNA, 5 end /clone=IMAGE-; 32520_at Cluster Incl. M24900:Human triiodothyronine recptor (THRA1, earl), and; 1904_at D50692 /FEATURE= /DEFINITION=HUMAMY1 Homo sapiens mRNA for c-myc binding; 1097_-sat L31584 /FEATURE=exon /DEFINITION=HUMEBI103 Human G protein-coupled rec; 734_at Mucin 4, Tracheobronchial; 586_s_at M31153 /FEATURE=cds /DEFINITION=HUMCYP178 Human steroid 17-alpha-hydrox; 494_at U31120 /FEATURE=mRNA /DEFINITION=HSU31120 Human interleukin-13 (IL-13) pr.

Metagene 203; 33947_at Cluster Incl. U18550:Human GPR3 G protein-coupled receptor gene, comple; 35707_at Cluster Incl. AI057614:oy31f04.x1 Homo sapiens cDNA, 3 end /clone=IMAG; 36037_g_at Cluster Incl. J05500:Human beta-spectrin (SPTB) mRNA, complete cds /c; 37924_g_at Cluster Incl. AA846749:aj99c10.s1 Homo sapiens cDNA, 3 end /clone=IM; 39054_at Cluster Incl. X08020:Human mRNA for glutathione S-transferase subunit 4; 39059_at Cluster Incl. AF034544:Homo sapiens delta7-sterol reductase mRNA, compl; 40107_at Cluster Incl. AF054987:Homo sapiens clone 23831 aldolase C mRNA, comple; 37371_at Cluster Incl. M81780:Homo sapiens acid sphingomyelinase (SMPD1) gene, c; 40912_s_at Cluster Incl. X81372:H. sapiens mRNA for biphenyl hydrolase-related pr; 804_s_at X86371 /FEATURE=cds /DEFINITION=HSRNAHUGL H. sapiens mRNA for tumour sup; 556_s_at M96233 /FEATURE=expanded_cds /DEFINITION=HUMGSTM4A Human glutathione tr.

Metagene 204; 32425_at Cluster Incl. U62431:Human nicotinic acetylcholine receptor alpha2 subu; 35090_g_at Cluster Incl. AB005060:Homo sapiens mRNA for NTAK, complete cds /cds=; 33561_at Cluster Incl. X80031:Homo sapiens COL4A3 mRNA /cds=(161,5173) /gb=X8003; 35382_at Cluster Incl. AF043244:Homo sapiens apoptosis repressor ARC (ARC) mRNA,; 36312_at Cluster Incl. L40377:Homo sapiens cytoplasmic antiproteinase 2 (CAP2) m; 38563_at Cluster Incl. AF039652:Homo sapiens ribonuclease H type II mRNA, comple; 39272_g_at Cluster Incl. AA461365:zx70e07.r1 Homo sapiens cDNA, 5 end /clone=IM; 33746_at Cluster Incl. D88208:Homo sapiens hSGT1 mRNA for hsgt1p, complete cds /; 36454_at Cluster Incl. AF037335:Homo sapiens carbonic anhydrase precursor (CA 12; 34373_at Cluster Incl. Z97054:Human DNA sequence from PAC 339A18 on chromosome X; 34816_at Cluster Incl. AI143868:qe04e11.x1 Homo sapiens cDNA, 3 end /clone=IMAG; 34871_at Cluster Incl. W30677:zb75h10.r1 Homo sapiens cDNA, 5 end /clone=IMAGE-; 36653_g_at Cluster Incl. J03824:Human uroporphyrinogen III synthase mRNA, comple; 177_at U38545 /FEATURE= /DEFINITION=HSU38545 Human ARF-activated phosphatidylcho.

Metagene 205; 41380_at Cluster Incl. AF053003:Homo sapiens diphthamide biosynthesis protein-2; 41403_at Cluster Incl. AI032612:ow17e07.x1 Homo sapiens cDNA, 3 end /clone=IMAG; 36001_at Cluster Incl. Y18643:Homo sapiens mRNA for methyltransferase-like prote; 39073_at Cluster Incl. AL038662:DKFZp566I0346_r1 Homo sapiens cDNA, 5 end /clon; 32751_at Cluster Incl. AF007140:Homo sapiens clone 23711 unknown mRNA, partial c; 37668_at Cluster Incl. M69039:Human pre-mRNA splicing factor SF2p32, complete se; 41321_s_at Cluster Incl. AA528077:nh90a11.s1 Homo sapiens cDNA, 3 end /clone=IM; 1985_s_at X73066 /FEATURE=cds /DEFINITION=HSNM23H1A H. sapiens NM23-H1 mRNA; 1521_at X17620 /FEATURE=mRNA /DEFINITION=HSNM23 Human mRNA for Nm23 protein, inv.

Metagene 206; 35118_at Cluster Incl. M12625:Human lecithin-cholesterol acyltransferase mRNA, c; 41083_at Cluster Incl. AC006276:Homo sapiens chromosome 19, cosmid R28379 /cds=(; 41084_at Cluster Incl. AI659108:tu08c09.x1 Homo sapiens cDNA, 3 end /clone=IMAG; 35621_at Cluster Incl. L77213:Homo sapiens phosphomevalonate kinase mRNA, comple; 36095_at Cluster Incl. N99340:IMAGE-20074 Homo sapiens cDNA /clone=IMAGE-20074 /; 37342_s_at Cluster Incl. AF070531:Homo sapiens clone 24764 mRNA sequence /cds=UN; 1274_s_at L22005 /FEATURE= /DEFINITION=HUMCDC34H Human ubiquitin conjugating enz.

Metagene 207; 31315_at Cluster Incl. D84143:Human immunoglobulin (mAb59) light chain V region; 34105_f_at Cluster Incl. AI147237:qb36f02.x1 Homo sapiens cDNA, 3 end /clone=IM; 32905_s_at Cluster Incl. M30038:Human tryptase mRNA, complete cds /cds=(17,844); 33499_s_at Cluster Incl. AF067420:Homo sapiens SNC73 protein (SNC73) mRNA, compl; 33500_i_at Cluster Incl. S71043:Ig alpha 2=immunoglobulin A heavy chain allotype; 33501_r_at Cluster Incl. S71043:Ig alpha 2=immunoglobulin A heavy chain allotype; 37864_s_at Cluster Incl. Y14737:Homo sapiens mRNA for immunoglobulin lambda heav; 38194_s_at Cluster Incl. M63438:Human Ig rearranged gamma chain mRNA, V-J-C regi; 32070_at Cluster Incl. X97267:H. sapiens LPAP gene /cds=(63,683) /gb=X97267 /gi=1; 33273_f_at Cluster Incl. X57809:Human rearranged immunoglobulin lambda light cha; 33274_f_at Cluster Incl. M18645:Human Ig rearranged lambda-chain mRNA VJC-region; 37219_at Cluster Incl. X72755:H. sapiens Humig mRNA /cds=(39,416) /gb=X72755 /gi=; 37965_at Cluster Incl. AA181053:zp66g11.s1 Homo sapiens cDNA, 3 end /clone=IMAG; 41164_at Cluster Incl. X67301:H. sapiens mRNA for IgM heavy chain constant region; 41165_g_at Cluster Incl. X67301:H. sapiens mRNA for IgM heavy chain constant regi; 41237_at Cluster Incl. D32129:Human mRNA for HLA class-I (HLA-A26) heavy chain,; 35338_at Cluster Incl. X17094:Human fur m-RNA for furin /cds=(216,2600) /gb=X1709; 37006_at Cluster Incl. AI660656:wf23c07.x1 Homo sapiens cDNA, 3 end /clone=IMAG; 41827_f_at Cluster Incl. AI932613:wo05c02.x1 Homo sapiens cDNA, 3 end /clone=IM; 32547_at Cluster Incl. X56253:Human MPR46 gene for 46 kd mannose 6-phosphate rece; 2009_at U33284 /FEATURE= /DEFINITION=HSU33284 Human protein tyrosine kinase PYK2.

Metagene 208; 31524_f_at Cluster Incl. Z80782:H. sapiens H2B/k gene /cds=(0,380) /gb=Z80782 /gi; 31528_f_at Cluster Incl. Z83738:H. sapiens hH2B/e gene /cds=(0,380) /gb=Z83738 /g; 34157_f_at Cluster Incl. AI200373:qf98c03.x1 Homo sapiens cDNA, 3 end /clone=IM; 35576_f_at Cluster Incl. AL009179:dJ97D16.4 (Histone H2B) /cds=(25,405) /gb=AL00; 32980_f_at Cluster Incl. AI688098:wc92f08.x1 Homo sapiens cDNA, 3 end /clone=IM; 32121_at Cluster Incl. U86453:Human phosphatidylinositol 3-kinase catalytic subu.

Metagene 209; 41007_at Cluster Incl. AF052497:Homo sapiens clone B18 unknown mRNA /cds=UNKNOWN; 33815_at Cluster Incl. J03626:Human UMP synthase mRNA, complete cds /cds=UNKNOWN; 34712_at Cluster Incl. AB023227:Homo sapiens mRNA for KIAA1010 protein, partial; 39783_at Cluster Incl. D43947:Human mRNA for KIAA0100 gene, complete cds /cds=(3; 32211_at Cluster Incl. AB009398:Homo sapiens mRNA for 26S proteasome subunit p40; 34776_at Cluster Incl. W27541:32c12 Homo sapiens cDNA /gb=W27541 /gi=1307345 /ug; 37032_at Cluster Incl. U08021:Human nicotinamide N-methyltransferase (NNMT) mRNA; 40948_at Cluster Incl. M96759:Human rod outer segment membrane protein 1 (ROM1).

Metagene 210; 31320_at Cluster Incl. U18548:Human GPR12 G protein coupled-receptor gene, compl; 31503_at Cluster Incl. W28732:50h7 Homo sapiens cDNA /gb=W28732 /gi=1308680 /ug=; 31720_s_at Cluster Incl. M10905:Human cellular fibronectin mRNA /cds=(0,2383) /g; 33002_at Cluster Incl. AF047487:Homo sapiens Nck-2 (NCK2) mRNA, complete cds /cd; 33064_at Cluster Incl. L07738:Homo sapiens DHP-sensitive calcium channel gamma s; 33647_s_at Cluster Incl. AA224768:nc12d09.r1 Homo sapiens cDNA /clone=IMAGE-1007; 35126_at Cluster Incl. AB026542:Homo sapiens WAVE2 mRNA for WASP-family protein,; 36338_at Cluster Incl. W28504:48e7 Homo sapiens cDNA /gb=W28504 /gi=1308515 /ug=; 36217_at Cluster Incl. Z35102:H. sapiens mRNA for Ndr protein kinase /cds=(595,19; 31800_at Cluster Incl. AL050136:Homo sapiens mRNA; cDNA DKFZp586L141 (from clone; 32038_s_at Cluster Incl. AI739308:wi30c12.x1 Homo sapiens cDNA, 3 end /clone=IM; 40452_at Cluster Incl. U83246:Homo sapiens copine I mRNA, complete cds /cds=(156; 38831_f_at Cluster Incl. AF053356:Homo sapiens chromosome 7q22 sequence /cds=(25; 39100_at Cluster Incl. X73608:H. sapiens mRNA for testican /cds=(434,1753) /gb=X7; 40634_at Cluster Incl. M86667:H. sapiens NAP (nucleosome assembly protein) mRNA,; 41552_g_at Cluster Incl. AW044624:wy78c04.x1 Homo sapiens cDNA, 3 end /clone=IM; 1928_s_at U78733 /FEATURE=mRNA#1 /DEFINITION=HSSMAD2S8 Homo sapiens mad protein; 1704_at S76992 /FEATURE= /DEFINITION=S76992 VAV2=VAV oncogene homolog [human, fe; 1588_at L42243 /FEATURE=cds#1 /DEFINITION=HUMIFNAM08 Homo sapiens (clone 51H8) a; 594_s_at M55265 /FEATURE=mRNA /DEFINITION=HUMACKII Human casein kinase II alpha; 359_at Y10659 /FEATURE=cds /DEFINITION=HSIL13RA H. sapiens IL-13Ra mRNA; 311_s_at Fibronectin, Alt. Splice 1; 248_at L40388 /FEATURE=mRNA /DEFINITION=HUMTRIP15M Homo sapiens thyroid receptor.

Metagene 211; 33001_s_at Cluster Incl. AF035960:Homo sapiens transglutaminase X mRNA, alternat; 38678_at Cluster Incl. AA733050:zg79b05.s1 Homo sapiens cDNA, 3 end /clone=3995; 37691_at Cluster Incl. X63380:Homo sapiens mRNA for serum response factor-relate; 651_at L07493 /FEATURE= /DEFINITION=HUM14RPA Homo sapiens replication protein A.

Metagene 212; 31759_at Cluster Incl. W26220:22d9 Homo sapiens cDNA /gb=W26220 /gi=1306631 /ug=; 35598_at Cluster Incl. AA904505:ol26b03.s1 Homo sapiens cDNA, 3 end /clone=IMAG; 35485_at Cluster Incl. X80818:H. sapiens mRNA for metabotropic glutamate receptor; 35950_at Cluster Incl. U90841:Homo sapiens SSX4 (SSX4) mRNA, complete cds /cds=(; 37801_at Cluster Incl. AF112972:Homo sapiens TJ6 mRNA, complete cds /cds=(5,2575; 37195_at Cluster Incl. M14565:Human cholesterol side-chain cleavage enzyme P450s; 38991_at Cluster Incl. U55980:HSU55980 Homo sapiens cDNA, 3 end /clone=25453 /c; 40501_s_at Cluster Incl. X73114:H. sapiens mRNA for slow MyBP-C /cds=(81,3452) /g; 39451_i_at Cluster Incl. AF050145:Homo sapiens iduronate-2-sulfatase (IDS) mRNA,; 1483_at L34059 /FEATURE= /DEFINITION=HUMCA4A Homo sapiens cadherin-4 mRNA, compl; 1024_at X02612 /FEATURE=expanded_cds /DEFINITION=HSCYP450 Human gene for cytochr.

Metagene 213; 32011_g_at Cluster Incl. Z82180:Human DNA sequence from clone E81G9 on chromosom; 33695_at Cluster Incl. AC004221:Homo sapiens DNA from chromosome 19, cosmid R291; 33237_at Cluster Incl. AB018344:Homo sapiens mRNA for KIAA0801 protein, complete; 36055_at Cluster Incl. AC003030:Homo sapiens chromosome 19, overlapping cosmids; 37172_at Cluster Incl. M75106:Human prepro-plasma carboxypeptidase B mRNA, compl; 38660_at Cluster Incl. F27891:HSPD16170 Homo sapiens cDNA /clone=s4000025D03 /gb; 39085_at Cluster Incl. M37984:Human slow twitch skeletal muscle/cardiac muscle t; 38729_at Cluster Incl. M88279:Human immunophilin (FKBP52) mRNA, complete cds /cd; 39500_s_at Cluster Incl. AL049299:Homo sapiens mRNA; cDNA DKFZp564P233 (from clo; 41556_s_at Cluster Incl. AF019386:Homo sapiens heparan sulfate 3-O-sulfotransfer; 1026_s_at U41068 /FEATURE=cds /DEFINITION=HSCOLLA5 Human collagen alpha2(XI) (CO.

Metagene 214; 38589_i_at Cluster Incl. M14630:Human prothymosin alpha mRNA, complete cds /cds=; 40661_at Cluster Incl. D78579:Homo sapiens mRNA for neuron derived orphan recept; 34398_at Cluster Incl. D86956:Human mRNA for KIAA0201 gene, complete cds /cds=(3; 37024_at Cluster Incl. AF010312:Homo sapiens Pig7 (PIG7) mRNA, complete cds /cds; 752_s_at D85429 /FEATURE=expanded_cds /DEFINITION=D85429 Homo sapiens gene for h; 452_at U66615 /FEATURE= /DEFINITION=HSU66615 Human SWI/SNF complex 155 KDa subun; 303_at Guanine Nucleotide Exchange Factor 1.

Metagene 215; 34690_at Cluster Incl. U66616:Human SWI/SNF complex 170 KDa subunit (BAF170) mRN; 39334_s_at Cluster Incl. L10377:Human (clone CTG-B37) mRNA sequence /cds=UNKNOWN.

Metagene 216; 32460_at Cluster Incl. S67368:GABRB2=gamma-aminobutyric acid A receptor beta 2 s; 33661_at Cluster Incl. U66589:Human ribosomal protein L5 pseudogene mRNA, comple; 32071_at Cluster Incl. AB002293:Human mRNA for KIAA0295 gene, partial cds /cds=(; 34709_r_at Cluster Incl. Z75331:H. sapiens mRNA for nuclear protein SA-2 /cds=(64; 34772_at Cluster Incl. AB023142:Homo sapiens mRNA for KIAA0925 protein, partial; 36891_at Cluster Incl. AL022237:bK1191B2.3.1 (PUTATIVE novel Acyl Transferase si; 37932_at Cluster Incl. Y12490:Homo sapiens mRNA for Golgi-associated microtubule; 38383_at Cluster Incl. U73338:Human methionine synthase mRNA, complete cds /cds=; 38753_at Cluster Incl. AF039022:Homo sapiens exportin t mRNA, complete cds /cds=; 1476_-sat U22376 /FEATURE=cds#5 /DEFINITION=HSU22376 Human (c-myb) gene, complet.

Metagene 217; 33583_r_at Cluster Incl. AA523313:ni41h09.s1 Homo sapiens cDNA, 3 end /clone=IM; 32310_f_at Cluster Incl. AI341574:qq94h09.x1 Homo sapiens cDNA, 3 end /clone=IM; 37151_at Cluster Incl. AF052120:Homo sapiens clone 23836 mRNA sequence /cds=UNKN; 37882_at Cluster Incl. X63468:H. sapiens mRNA for transcription factor TFIIE alph; 36462_at Cluster Incl. U50383:Human retinoic acid-responsive protein (NN8-4AG) m; 36498_at Cluster Incl. AI936759:wp69b12.x1 Homo sapiens cDNA, 3 end /clone=IMAG; 36888_at Cluster Incl. AB020648:Homo sapiens mRNA for KIAA0841 protein, partial; 39762_at Cluster Incl. AB007885:Homo sapiens KIAA0425 mRNA, complete cds /cds=(1; 38468_at Cluster Incl. U65676:Human Hermansky-Pudlak syndrome protein (HPS) mRNA.

Metagene 218; 31523_f_at Cluster Incl. Z80780:H. sapiens H2B/h gene /cds=(0,380) /gb=Z80780 /gi; 31663_at Cluster Incl. AB016902:Homo sapiens HGC6.3 mRNA, complete cds /cds=(287; 31727_at Cluster Incl. U91510:Human CD39L1 mRNA, complete cds /cds=(21,1439) /gb; 33472_at Cluster Incl. Z11737:H. sapiens mRNA for flavin-containing monooxygenase; 36759_at Cluster Incl. U29589:Human m3 muscarinic acetylcholine receptor (CHRM3); 31857_r_at Cluster Incl. D50924:Human mRNA for KIAA0134 gene, complete cds /cds=; 33251_at Cluster Incl. AB018322:Homo sapiens mRNA for KIAA0779 protein, partial; 36458_at Cluster Incl. AB023235:Homo sapiens mRNA for KIAA1018 protein, complete; 34352_at Cluster Incl. AA631698:np79a08.s1 Homo sapiens cDNA /clone=IMAGE-113250; 39560_at Cluster Incl. H10776:ym07h11.r1 Homo sapiens cDNA, 5 end /clone=IMAGE-; 40984_at Cluster Incl. W28255:44b8 Homo sapiens cDNA /gb=W28255 /gi=1308203 /ug=; 41842_at Cluster Incl. AI701156:we10f09.x1 Homo sapiens cDNA, 3 end /clone=IMAG; 1840_g_at Ras-Like Protein Tc4; 526_s_at U13696 /FEATURE=cds /DEFINITION=HSU13696 Human homolog of yeast mutL (h; 323_at Serine Kinase Psk-H1.

Metagene 219; 31519_f_at Cluster Incl. M90356:Human BTF3 protein homologue gene, complete cds; 31932_f_at Cluster Incl. M90357:Human basic transcription factor 3a (BTF3a) gene; 32334_f_at Cluster Incl. AB009010:Homo sapiens mRNA for polyubiquitin UbC, compl; 32335_r_at Cluster Incl. AB009010:Homo sapiens mRNA for polyubiquitin UbC, compl; 38476_at Cluster Incl. L13434:Human chromosome 3p21.1 gene sequence, complete cd; 39844_at Cluster Incl. AI806379:wf27b10.x1 Homo sapiens cDNA, 3 end /clone=IMAG; 1256_at L38929 /FEATURE=mRNA /DEFINITION=HUMPTPD Homo sapiens protein tyrosine p.

Metagene 220; 31475_at Cluster Incl. AF082559:Homo sapiens truncated TRF1-interacting ankyrin-; 33987_at Cluster Incl. M36340:Human ADP-ribosylation factor 1 (ARF1) mRNA, compl; 35652_g_at Cluster Incl. AF002715:Homo sapiens MAP kinase kinase kinase (MTK1) m; 38655_at Cluster Incl. AI525633:PT1.3_{—}04_A08.r Homo sapiens cDNA, 5 end /clone_{—}; 38712_at Cluster Incl. AL035291:H. sapiens gene from PACs 125H23 and 105D12 /cds=; 39348_at Cluster Incl. X99209:H. sapiens mRNA for arginine methyltransferase /cds; 41151_at Cluster Incl. U45973:Human phosphatidylinositol (4,5)bisphosphate 5-pho; 32826_at Cluster Incl. AJ133133:Homo sapiens mRNA for ecto-ATP diphosphohydrolas; 32832_at Cluster Incl. AF084928:Homo sapiens erythroblast macrophage protein EMP; 33409_at Cluster Incl. AA158243:zo76c01.s1 Homo sapiens cDNA, 3 end /clone=IMAG; 33444_at Cluster Incl. D30756:Human mRNA for KIAA0049 gene, complete cds /cds=(1; 33922_at Cluster Incl. U17838:Human zinc finger protein RIZ mRNA, complete cds /; 35273_at Cluster Incl. AF007151:Homo sapiens clone 23967 unknown mRNA, partial c; 39162_at Cluster Incl. AA156987:zl19b05.s1 Homo sapiens cDNA, 3 end /clone=IMAG; 40175_at Cluster Incl. AI141670:ot08b12.x1 Homo sapiens cDNA, 3 end /clone=IMAG; 32565_at Cluster Incl. U66619:Human SWI/SNF complex 60 KDa subunit (BAF60c) mRNA; 2053_at M34064 /FEATURE= /DEFINITION=HUMNCADH Human N-cadherin mRNA, complete cd; 487_g_at U60521 /FEATURE= /DEFINITION=HSU60521 Human protease proMch6 (Mch6) mRN; 456_at U66619 /FEATURE= /DEFINITION=HSU66619 Human SWI/SNF complex 60 KDa subuni.

Metagene 221; 36747_at Cluster Incl. AF052172:Homo sapiens clone 24617 mRNA sequence /cds=UNKN; 39288_at Cluster Incl. AI951798:wx37f11.x1 Homo sapiens cDNA, 3 end /clone=IMAG; 40484_g_at Cluster Incl. U49857:Human transcriptional activator mRNA, complete c; 33436_at Cluster Incl. Z46629:Homo sapiens SOX9 mRNA /cds=(359,1888) /gb=Z46629.

Metagene 222; 32648_at Cluster Incl. U15979:Human (dlk) mRNA, complete cds /cds=(173,1321) /gb; 35706_at Cluster Incl. N32859:yw88d02.s1 Homo sapiens cDNA, 3 end /clone=IMAGE-; 38315_at Cluster Incl. AB015228:Homo sapiens mRNA for RALDH2-T, complete cds /cd; 41505_r_at Cluster Incl. AF055376:Homo sapiens short form transcription factor C.

Metagene 223; 37648_at Cluster Incl. D63487:Human mRNA for KIAA0153 gene, partial cds /cds=(0,; 37892_at Cluster Incl. J04177:Human alpha-1 type XI collagen (COL11A1) mRNA, com; 33907_at Cluster Incl. AF012072:Homo sapiens eIF4GII mRNA, complete cds /cds=(25; 33101_g_at Cluster Incl. AB017551:Homo sapiens mRNA for 16G2, complete cds /cds=.

Metagene 224; 32969_r_at Cluster Incl. Y12661:H. sapiens vgf gene /cds=(212,2062) /gb=Y12661 /g; 34495_r_at Cluster Incl. AJ011733:Homo sapiens mRNA for synaptogyrin 4 protein /; 35887_at Cluster Incl. L34219:Homo sapiens retinaldehyde-binding protein (CRALBP; 38166_r_at Cluster Incl. AF010403:Homo sapiens ALR mRNA, complete cds /cds=(0,15; 40163_r_at Cluster Incl. AC003107:Human DNA from chromosome 19-specific cosmid R; 39157_at Cluster Incl. AL021937:dJ149A16.5 (PUTATIVE novel protein similar to mo; 39177_r_at Cluster Incl. M54994:Human bile salt-activated lipase (BAL) mRNA, com; 39859_r_at Cluster Incl. AF071504:Homo sapiens syntaxin 11 mRNA, complete cds /c; 40172_g_at Cluster Incl. AF062739:Homo sapiens GSK-3 binding protein FRAT2 (FRAT; 40930_at Cluster Incl. W25958:18h6 Homo sapiens cDNA /gb=W25958 /gi=1306360 /ug=; 1028_at U43431 /FEATURE= /DEFINITION=HSU43431 Human DNA topoisomerase III mRNA,.

Metagene 225; 38937_at Cluster Incl. AF022152:Homo sapiens AP-3 complex beta3B subunit mRNA, c; 40329_at Cluster Incl. AL031228:dJ1033B10.10 (membrane protein with histidine ri; 36822_at Cluster Incl. U51334:Human putative RNA binding protein (RBP56) mRNA, c; 38663_at Cluster Incl. AI033692:ow26f02.x1 Homo sapiens cDNA, 3 end /clone=IMAG; 39079_at Cluster Incl. D85758:Homo sapiens mRNA for human protein homologous to; 32761_at Cluster Incl. AB002322:Human mRNA for KIAA0324 gene, partial cds /cds=(; 34836_at Cluster Incl. U18420:Human ras-related small GTP binding protein Rab5 (; 36161_at Cluster Incl. M34175:Human beta adaptin mRNA, complete cds /cds=(177,29; 38122_at Cluster Incl. D87075:Human mRNA for KIAA0238 gene, partial cds /cds=(0,; 40636_at Cluster Incl. AI807620:wf49b01.x1 Homo sapiens cDNA, 3 end /clone=IMAG; 1997_s_at U19599 /FEATURE= /DEFINITION=HSU19599 Human (BAX delta) mRNA, complete; 508_at U43923 /FEATURE= /DEFINITION=HSU43923 Human transcription factor SUPT4H m.

Metagene 226; 34610_at Cluster Incl. W25845:13h9 Homo sapiens cDNA /gb=W25845 /gi=1305968 /ug=; 32965_f_at Cluster Incl. W28645:52e8 Homo sapiens cDNA /gb=W28645 /gi=1308800 /u; 36696_at Cluster Incl. AB000359:Homo sapiens PIGCP1 pseudogene /cds=(0,416) /gb=; 38901_at Cluster Incl. AB020698:Homo sapiens mRNA for KIAA0891 protein, partial; 39285_at Cluster Incl. L28957:Homo sapiens CTP-phosphocholine cytidyltransferase; 39290_f_at Cluster Incl. W28257:44c1 Homo sapiens cDNA /gb=W28257 /gi=1308205 /u; 41619_at Cluster Incl. AL022398:dJ434O14.4 (Interferon Regulatory Factor 6) /cds; 34692_r_at Cluster Incl. AF006087:Homo sapiens Arp2/3 protein complex subunit p2; 35201_at Cluster Incl. X16135:Human mRNA for novel heterogeneous nuclear RNP pro; 35204_at Cluster Incl. U52840:Homo sapiens semaphorin F homolog mRNA, complete c; 39024_at Cluster Incl. AF042357:AF042357 Homo sapiens cDNA /gb=AF042357 /gi=4104; 39399_at Cluster Incl. AJ006417:Homo sapiens mRNA for beta-tubulin folding cofac; 40442_f_at Cluster Incl. W26019:18b9 Homo sapiens cDNA /gb=W26019 /gi=1306304 /u; 41199_s_at Cluster Incl. W27050:19f7 Homo sapiens cDNA /gb=W27050 /gi=1306422 /u; 32752_at Cluster Incl. W72440:zd65e10.s1 Homo sapiens cDNA, 3 end /clone=IMAGE-; 35345_at Cluster Incl. X83618:H. sapiens mRNA for 3-hydroxy-3-methylglutaryl coen; 38029_at Cluster Incl. J02939:Human membrane glycoprotein 4F2 antigen heavy chai; 38127_at Cluster Incl. Z48199:H. sapiens syndecan-1 gene (exons 2-5) /cds=(0,866); 38412_at Cluster Incl. U53588:Homo sapiens MHC class 1 region /cds=(199,579) /gb; 39464_at Cluster Incl. W28493:47h11 Homo sapiens cDNA /gb=W28493 /gi=1308459 /ug; 39539_at Cluster Incl. AI684866:wa86e09.x1 Homo sapiens cDNA, 3 end /clone=IMAG; 39816_g_at Cluster Incl. AA883101:am24d05.s1 Homo sapiens cDNA, 3 end /clone=IM; 40186_at Cluster Incl. Y08302:H. sapiens mRNA for MAP kinase phosphatase 4 /cds=(; 40886_at Cluster Incl. L41498:Homo sapiens longation factor 1-alpha 1 (PTI-1) mR; 40888_f_at Cluster Incl. W28170:43a12 Homo sapiens cDNA /gb=W28170 /gi=1308118 /; 41301_at Cluster Incl. W28608:49b1 Homo sapiens cDNA /gb=W28608 /gi=1308556 /ug=; 33105_at Cluster Incl. W28790:54g3 Homo sapiens cDNA /gb=W28790 /gi=1308945 /ug=; 33110_at Cluster Incl. AI356682:qy22a10.x1 Homo sapiens cDNA, 3 end /clone=IMAG; 33211_at Cluster Incl. AW051889:wz04f05.x1 Homo sapiens cDNA, 3 end /clone=IMAG; 2013_at U35117 /FEATURE= /DEFINITION=HSU35117 Human transcription factor Dp-2 mR; 1644_at U36764 /FEATURE= /DEFINITION=HSU36764 Human TGF-beta receptor interactin; 1565_s_at M96995 /FEATURE= /DEFINITION=HUMEGFGRBA Homo sapiens epidermal growth; 1358_s_at U22970 /FEATURE=mRNA#1 /DEFINITION=HSU22970 Human interferon-inducible; 1240_at U13022 /FEATURE= /DEFINITION=HSU13022 Human negative regulator of progra; 1142_at Fibroblast Growth Factor Receptor K-Sam, Alt. Splice 1; 945_at D50063 /FEATURE= /DEFINITION=HUMP40MOV Human mRNA for proteasome subunit; 725_i_at Chorionic Somatomammotropin Hormone Cs-5; 540_at S67070 /FEATURE= /DEFINITION=S67070 heat shock protein HSP72 homolog [hum.

Metagene 227; 34948_at Cluster Incl. AI820065:wg56a09.x1 Homo sapiens cDNA, 3 end /clone=IMAG; 34254_at Cluster Incl. U14417:Human Ral guanine nucleotide dissociation stimulat; 39407_at Cluster Incl. M22488:Human bone morphogenetic protein 1 (BMP-1) mRNA /c; 36642_at Cluster Incl. J00287:Human pepsinogen gene /cds=(55,1221) /gb=J00287 /g; 39564_s_at Cluster Incl. C20962:HUMGS0002427 Homo sapiens cDNA, 3 end /clone-en; 1516_g_at Rad2; 1185_at D49410 /FEATURE=expanded_cds /DEFINITION=HUMIL3RA12 Human gene for inter; 852_at S70348 /FEATURE= /DEFINITION=S70348 integrin beta 3 {alternatively splice.

Metagene 228; 36311_at Cluster Incl. U40370:Human 3,5 cyclic nucleotide phosphodiesterase (HS; 37461_at Cluster Incl. AF004327:Homo sapiens angiopoietin-2 mRNA, complete cds /; 39315_at Cluster Incl. D13628:Human mRNA for KIAA0003 gene, complete cds /cds=(9; 1951_at AF004327 /FEATURE= /DEFINITION=AF004327 Homo sapiens angiopoietin-2 mRNA.

Metagene 229; 32289_at Cluster Incl. U40317:Human protein tyrosine phosphatase PTPsigma (PTIsi; 33569_at Cluster Incl. D50532:Homo sapiens mRNA for macrophage lectin 2, complet; 34897_at Cluster Incl. W26524:32g4 Homo sapiens cDNA /gb=W26524 /gi=1307385 /ug=; 39968_at Cluster Incl. U50136:Human leukotriene C4 synthase (LTC4S) gene, comple; 32725_at Cluster Incl. AF042083:Homo sapiens BH3 interacting domain death agonis; 36052_at Cluster Incl. U43959:Human beta 4 adducin mRNA, alternatively spliced p; 32238_at Cluster Incl. AF001383:Homo sapiens amphiphysin II mRNA, complete cds /; 32764_at Cluster Incl. AI796048:wh41g06.x1 Homo sapiens cDNA, 3 end /clone=IMAG; 40280_at Cluster Incl. U72508:Human B7 mRNA, complete cds /cds=(112,1050) /gb=U7; 1595_at L06139 /FEATURE= /DEFINITION=HUMTEKRPTK Homo sapiens receptor protein-ty.

Metagene 230; 31874_at Cluster Incl. Y07846:H. sapiens mRNA for GAR22 protein /cds=(132,1145) /; 35619_at Cluster Incl. AB014534:Homo sapiens mRNA for KLAA0634 protein, partial; 36880_at Cluster Incl. U07736:Human quinone oxidoreductase2 (NQO2) gene /cds=(27; 37266_at Cluster Incl. U69645:Human zinc finger protein mRNA, complete cds /cds=; 38313_at Cluster Incl. AB028985:Homo sapiens mRNA for KIAA1062 protein, partial; 39712_at Cluster Incl. AI541308:pec1.2-4.F11.r Homo sapiens cDNA, 5 end /clone_{—}; 40127_at Cluster Incl. M95929:Human homeobox protein (PHOX1) mRNA, 3 end /cds=(; 32176_at Cluster Incl. AB011110:Homo sapiens mRNA for KIAA0538 protein, partial; 34408_at Cluster Incl. AF004222:Homo sapiens RTN2-A (RTN2) mRNA, complete cds /c; 38761_s_at Cluster Incl. AA487755:ab13f11.r1 Homo sapiens cDNA, 5 end /clone=IM; 2052_g_at M31767 /FEATURE=mRNA /DEFINITION=HUMDNAMET Human 06-methylguanine-DNA; 1099_s_at L38503 /FEATURE= /DEFINITION=HUMGSTT2A Homo sapiens glutathione S-tran; 355_s_at D38037 /FEATURE= /DEFINITION=HUMOTK4 Human mRNA for FK506-binding prote.

Metagene 231; 31605_at Cluster Incl. U72518:Human destrin-2 pseudogene mRNA, complete cds /cds; 35377_at Cluster Incl. AL080159:Homo sapiens mRNA; cDNA DKFZp434M154 (from clone; 38173_at Cluster Incl. AB028999:Homo sapiens mRNA for KIAA1076 protein, partial; 41098_at Cluster Incl. AB002379:Human mRNA for KIAA0381 gene, partial cds /cds=(; 41449_at Cluster Incl. AJ000534:Homo sapiens mRNA for epsilon-sarcoglycan /cds=(; 34187_at Cluster Incl. D28483:Human scr3 mRNA for RNA binding protein SCR3, comp; 36053_at Cluster Incl. AF041248:Homo sapiens cyclin-dependent kinase inhibitor (; 37203_at Cluster Incl. L07765:Human carboxylesterase mRNA, complete cds /cds=(67; 38974_at Cluster Incl. AF021819:Homo sapiens RNA-binding protein regulatory subu; 32163_f_at Cluster Incl. AA216639:zq95f07.s1 Homo sapiens cDNA, 3 end /clone=IM; 33884_s_at Cluster Incl. AB014584:Homo sapiens mRNA for KIAA0684 protein, partia; 41514_s_at Cluster Incl. W26628:34a4 Homo sapiens cDNA /gb=W26628 /gi=1307471 /u; 1879_at M14949 /FEATURE=cds /DEFINITION=HUMRASR2 Human R-ras gene, exons 2 throu; 1802_s_at X03363 /FEATURE=cds /DEFINITION=HSERB2R Human c-erb-B-2 mRNA; 1132_s_at L12060 /FEATURE=mRNA /DEFINITION=HUMRARG7A Homo sapiens retinoic acid; 568_at M80335 /FEATURE= /DEFINITION=HUMPKACATO Homo sapiens protein kinase A cat

Metagene 232; 31645_at Cluster Incl. AB020625:Homo sapiens mRNA for butyrophilin like receptor; 31967_at Cluster Incl. AF035835:Homo sapiens nephrin (NPHS1) mRNA, complete cds; 33594_at Cluster Incl. AB017788:Homo sapiens hdkk-4 mRNA, complete cds /cds=(0,6; 32882_at Cluster Incl. AF059203:Homo sapiens acyl coenzyme A-cholesterol acyltra; 33520_at Cluster Incl. M13232:Human factor VII serine protease precursor mRNA, c; 34475_at Cluster Incl. L34408:Homo Sapiens (clone B3B3E13) chromosome 4p16.3 DNA; 34999_at Cluster Incl. AF060231:Homo sapiens herpesvirus entry protein C (HVEC); 35427_at Cluster Incl. W28256:44b9 Homo sapiens cDNA /gb=W28256 /gi=1308204 /ug=; 38943_at Cluster Incl. U36787:Human putative holocytochrome c-type synthetase mR; 39569_at Cluster Incl. U72849:untitled /cds=(98,6199) /gb=U72849 /gi=4097997 /ug; 40681_at Cluster Incl. AB008375:Homo sapiens mRNA for osteoblast specific cystei; 41469_at Cluster Incl. L10343:Huma elafin gene, complete cds /cds=(516,869) /gb=; 36558_at Cluster Incl. M92302:Human voltage-dependent calcium channel beta-1 sub; 40408_at Cluster Incl. L06845:Human cysteinyl-tRNA synthetase mRNA, partial cds; 40849_s_at Cluster Incl. U88528:Human transcription factor LZIP mRNA, complete c; 32831_at Cluster Incl. AA453183:zx46b09.r1 Homo sapiens cDNA, 5 end /clone=IMAG; 36127_g_at Cluster Incl. U18919:Human chromosome 17q12-21 mRNA, clone pOV-2, par; 36997_at Cluster Incl. J04809:Human cytosolic adenylate kinase (AK1) gene, compl; 39503_s_at Cluster Incl. AB006713:Homo sapiens mRNA for dihydropyrimidinase rela; 1902_at M13194 /FEATURE=mRNA /DEFINITION=HUMERCC1 Human excision repair protein; 1497_at L04270 /FEATURE= /DEFINITION=HUMTNFRRP Homo sapiens (clone CD18) tumor n; 702_f_at Homeotic Protein Hpx-5.

Metagene 233; 36266_at Cluster Incl. U79275:Human clone 23947 mRNA, partial cds /cds=(0,401) /; 37174_at Cluster Incl. D14660:Human mRNA for KIAA0104 gene, complete cds /cds=(3; 36625_at Cluster Incl. L40401:Homo sapiens (clone zap128) mRNA, 3 end of cds /c.

Metagene 234; 41215_s_at Cluster Incl. D13891:Human mRNA for Id-2H, complete cds /cds=(96,500); 36591_at Cluster Incl. X06956:Human HALPHA44 gene for alpha-tubulin, exons 1-3 /; 36617_at Cluster Incl. X77956:H. sapiens Id1 mRNA /cds=(35,499) /gb=X77956 /gi=45; 36618_g_at Cluster Incl. X77956:H. sapiens Id1 mRNA /cds=(35,499) /gb=X77956 /gi=; 36619_r_at Cluster Incl. S78825:Id1 (Id1-b)=transcription regulator helix-loop-h; 37043_at Cluster Incl. AL021154:dJ150O5.2 (Inhibitor of DNA binding 3 (dominant; 38755_at Cluster Incl. X84709:H. sapiens mRNA for mediator of receptor-induced to; 1955_s_at AF035528 /FEATURE= /DEFINITION=AF035528 Homo sapiens Smad6 mRNA, compl; 1857_at AF010193 /FEATURE= /DEFINITION=AF010193 Homo sapiens MAD-related gene SM.

Metagene 235; 34078_s_at Cluster Incl. M61854:Human cytochrome P4502C19 (CYP2C19) mRNA, clone; 35087_at Cluster Incl. AF007147:Homo sapiens clone 23712 mRNA sequence /cds=UNKN; 36766_at Cluster Incl. X55988:Human EDN mRNA for eosinophil derived neurotoxin /; 33263_at Cluster Incl. X67098:H. sapiens rTS alpha mRNA containing four open read; 33763_at Cluster Incl. AI829671:wf09b02.x1 Homo sapiens cDNA, 3 end /clone=IMAG; 40770_f_at Cluster Incl. AB017019:Homo sapiens mRNA for JKTBP2, complete cds /cd; 318_at D64142 /FEATURE=mRNA /DEFINITION=D64142 Human mRNA for histone H1x, compl; 319_g_at D64142 /FEATURE=mRNA /DEFINITION=D64142 Human mRNA for histone H1x, com.

Metagene 236; 35109_at Cluster Incl. AB018299:Homo sapiens mRNA for KIAA0756 protein, partial; 37500_at Cluster Incl. D50419:Homo sapiens mRNA for OTK18, complete cds /cds=(34; 41411_at Cluster Incl. AI566877:tn24f02.x1 Homo sapiens cDNA, 3 end /clone=IMAG; 40070_at Cluster Incl. D50912:Human mRNA for KIAA0122 gene, partial cds /cds=(0,; 32776_at Cluster Incl. M35416:Human GTP-binding protein (RALB) mRNA, complete cd; 39484_at Cluster Incl. W28518:48a1 Homo sapiens cDNA /gb=W28518 /gi=1308466 /ug=; 1356_at U18321 /FEATURE= /DEFINITION=HSU18321 Human ionizing radiation resistanc; 1273_r_at L22005 /FEATURE= /DEFINITION=HUMCDC34H Human ubiquitin conjugating enz.

Metagene 237; 31439_f_at Cluster Incl. X63095:H. sapiens mRNA for rhesus polypeptide (RhVI) /cd; 32480_at Cluster Incl. X07495:Human mRNA for cp19 homeobox from HOX-3 locus /cds; 34577_at Cluster Incl. U10694:Human MAGE-9 antigen (MAGE9) gene, complete cds /c; 36378_at Cluster Incl. AF085807:Homo sapiens uroplakin Ia mRNA, partial cds /cds; 34902_at Cluster Incl. AB007961:Homo sapiens mRNA, chromosome 1 specific transcr; 36764_at Cluster Incl. AC004125:Homo sapiens Chromosome 16 BAC clone CIT987SK-62; 38491_at Cluster Incl. U11732:Human ets-like gene (tel) mRNA, complete cds /cds=; 34189_at Cluster Incl. D31891:Human mRNA for KIAA0067 gene, complete cds /cds=(8; 40491_at Cluster Incl. W84531:zd90h11.s1 Homo sapiens cDNA, 3 end /clone=IMAGE-; 35254_at Cluster Incl. AB007447:Homo sapiens mRNA for Fln29, complete cds /cds=(; 666_at L20965 /FEATURE= /DEFINITION=HUMPDEA Human phosphodiesterase mRNA, comple.

Metagene 238; 36417_s_at Cluster Incl. AF035295:Homo sapiens clone 23623 mRNA, partial cds /cd; 36224_g_at Cluster Incl. AI827895:wf12b02.x1 Homo sapiens cDNA, 3 end /clone=IM; 31807_at Cluster Incl. U69190:U69190 Homo sapiens cDNA /clone=27655 /gb=U69190 /; 33300_at Cluster Incl. AL031282:dJ283E3.3.1 (Cell Division Cycle 2-Like 2 (PITSL; 35135_at Cluster Incl. X13956:Human 12S RNA induced by poly(rI), poly(rc) and Ne; 35629_at Cluster Incl. AL022238:dJ1042K10.3 (novel protein) /cds=(0,731) /gb=AL0; 36881_at Cluster Incl. X71129:H. sapiens mRNA for electron transfer flavoprotein; 38718_at Cluster Incl. AL050101:Homo sapiens mRNA; cDNA DKFZp586E1519 (from clon; 38723_at Cluster Incl. AF052137:Homo sapiens clone 23918 mRNA sequence /cds=UNKN; 39747_at Cluster Incl. U52427:Human RNA polymerase II seventh subunit (rpb-7) ge; 34365_at Cluster Incl. AF042386:Homo sapiens cyclophilin-33B (CYP-33) mRNA, comp; 35779_at Cluster Incl. AJ133421:Homo sapiens mRNA for leucocyte vacuolar protein; 35780_at Cluster Incl. AF035292:Homo sapiens clone 23584 mRNA sequence /cds=UNKN; 36166_at Cluster Incl. AF083190:Homo sapiens SPF31 (SPF31) mRNA, complete cds /c; 36594_s_at Cluster Incl. U72263:Human multiple exostoses type II protein EXT2.I; 36649_at Cluster Incl. X66113:H. sapiens mRNA for PM/Scl 100 kD nucleolar protein; 36955_at Cluster Incl. U10362:Human GP36b glycoprotein mRNA, complete cds /cds=(; 37325_at Cluster Incl. D14697:Human mRNA for KIAA0003 gene, complete cds /cds=(1; 38373_g_at Cluster Incl. U66042:Human clone 191B7 placenta expressed mRNA from c; 39086_g_at Cluster Incl. AA768912:nz82h06.s1 Homo sapiens cDNA /clone=IMAGE-1302; 40905_s_at Cluster Incl. AL050369:Homo sapiens mRNA; cDNA DKFZp566J153 (from clo; 2064_g_at L20046 /FEATURE= /DEFINITION=HUMERCC5A Human ERCC5 excision repair pro; 745_at D50495 /FEATURE=mRNA /DEFINITION=HUMTEF Homo sapiens mRNA for transcripti; 465_at U74667 /FEATURE= /DEFINITION=HSU74667 Human tat interactive protein (TIP6.

Metagene 239; 34242_at Cluster Incl. AB014581:Homo sapiens mRNA for KIAA0681 protein, partial; 800_g_at X80343 /FEATURE=cds /DEFINITION=HSP35R H. sapiens p35 mRNA for regulator.

Metagene 240; 37113_at Cluster Incl. AF022795:Homo sapiens TGF beta receptor associated protei.

Metagene 241; 33694_at Cluster Incl. L40933:Homo sapiens phosphoglucomutase-related protein (P; 35916_s_at Cluster Incl. AA877215:ob15e02.s1 Homo sapiens cDNA, 3 end /clone=IM; 33246_at Cluster Incl. AA521481:aa69h09.s1 Homo sapiens cDNA, 3 end /clone=IMAG; 34184_at Cluster Incl. AB012162:Homo sapiens mRNA for APCL protein, complete cds; 36489_at Cluster Incl. D00860:Homo sapiens mRNA for phosphoribosyl pyrophosphate; 1649_at U61836 /FEATURE=mRNA /DEFINITION=HSU61836 Human putative cyclin G1 inter.

Metagene 242; 36234_at Cluster Incl. U79273:Human clone 23933 mRNA sequence /cds=UNKNOWN /gb=U; 39301_at Cluster Incl. X85030:H. sapiens mRNA for skeletal muscle-specific calpai; 38352_at Cluster Incl. AF016371:Homo sapiens U-snRNP-associated cyclophilin (USA; 34778_at Cluster Incl. AA418080:zv97h07.s1 Homo sapiens cDNA, 3 end /clone=IMAG; 35763_at Cluster Incl. AB011112:Homo sapiens mRNA for KIAA0540 protein, partial; 39914_r_at Cluster Incl. W28976:54e5 Homo sapiens cDNA /gb=W28976 /gi=1308924 /u; 40215_at Cluster Incl. D50840:Homo sapiens mRNA for ceramide glucosyltransferase; 306_s_at J02621 /FEATURE=mRNA /DEFINITION=HUMHMG14 Human non-histone chromosomal.

Metagene 243; 37484_at Cluster Incl. X68742:H. sapiens mRNA for integrin, alpha subunit /cds=UN; 37805_at Cluster Incl. AF060567:Homo sapiens sushi-repeat protein (SRPUL) mRNA,; 38592_s_at Cluster Incl. AI828210:wk81e09.x1 Homo sapiens cDNA, 3 end /clone=IM; 39307_s_at Cluster Incl. X81637:H. sapiens clathrin light chain b gene /cds=UNKNO; 39631_at Cluster Incl. U52100:Human XMP mRNA, complete cds /cds=(63,566) /gb=U52; 41107_at Cluster Incl. AB002372:Human mRNA for KIAA0374 gene, complete cds /cds=; 33736_at Cluster Incl. Y16522:Homo sapiens mRNA for hSLP-1 protein /cds=(44,1228; 35668_at Cluster Incl. AJ001014:Homo sapiens mRNA encoding RAMP1 /cds=(32,478) /; 35704_at Cluster Incl. X92814:H. sapiens mRNA for rat HREV107-like protein /cds=(; 36023_at Cluster Incl. AI864120:wg64a06.x1 Homo sapiens cDNA, 3 end /clone=IMAG; 36025_at Cluster Incl. AB002335:Human mRNA for KIAA0337 gene, complete cds /cds=; 36452_at Cluster Incl. AB028952:Homo sapiens mRNA for KIAA1029 protein, complete; 36492_at Cluster Incl. AI347155:tc04c11.x1 Homo sapiens cDNA, 3 end /clone=IMAG; 36540_at Cluster Incl. AB018260:Homo sapiens mRNA for KIAA0717 protein, partial; 36811_at Cluster Incl. U24389:Human lysyl oxidase-like protein gene /cds=(446,21; 37192_at Cluster Incl. U28389:Human dematin 52 kDa subunit mRNA, complete cds /c; 37244_at Cluster Incl. AA746355:oa56f02.r1 Homo sapiens cDNA /clone=IMAGE-130898; 38004_at Cluster Incl. X96753:H. sapiens mRNA for melanoma-associated chondroitin; 38609_at Cluster Incl. L34355:Homo sapiens (clone p4) 50 kD dystrophin-associate; 38642_at Cluster Incl. Y10183:H. sapiens mRNA for MEMD protein /cds=(0,1748) /gb=; 39031_at Cluster Incl. AA152406:zo07f01.s1 Homo sapiens cDNA, 3 end /clone=IMAG; 39058_at Cluster Incl. U01147:Human guanine nucleotide regulatory protein (ABR); 39080_at Cluster Incl. M88458:Human ELP-1 mRNA sequence /cds=UNKNOWN /gb=M88458; 41775_at Cluster Incl. AF064084:Homo sapiens prenylcysteine carboxylmethyltrans; 33346_r_at Cluster Incl. M61764:Human gamma-tubulin mRNA, complete cds /cds=(24,; 33433_at Cluster Incl. AL049943:Homo sapiens mRNA; cDNA DKFZp564F0522 (from clon; 33929_at Cluster Incl. X54232:Human mRNA for heparan sulfate proteaglycan (glypi; 34371_at Cluster Incl. U79267:Human clone 23840 mRNA, partial cds /cds=(0,521) /; 34376_at Cluster Incl. AB019517:Homo sapiens PKIG mRNA for protein kinase inhibi; 34403_at Cluster Incl. U58516:Human breast epithelial antigen BA46 mRNA, complet; 34865_at Cluster Incl. AI360249:qy84f07.x1 Homo sapiens cDNA, 3 end /clone=IMAG; 36683_at Cluster Incl. AI953789:wx69d10.x1 Homo sapiens cDNA, 3 end /clone=IMAG; 36989_at Cluster Incl. L19711:Human dystroglycan (DAG1) mRNA, complete cds /cds=; 39842_at Cluster Incl. AF059293:Homo sapiens cytokine-like factor-1 precursor (C; 40196_at Cluster Incl. D88153:Homo sapiens mRNA for HYA22, complete cds /cds=(14; 40587_s_at Cluster Incl. AF054186:Homo sapiens p18 protein mRNA, complete cds /c; 32523_at Cluster Incl. M20470:Human lymphocyte clathrin light-chain B mRNA, comp; 32610_at Cluster Incl. X93510:H. sapiens mRNA for 37 kDa LIM domain protein /cds=; 33128_s_at Cluster Incl. W68521:zd36f07.r1 Homo sapiens cDNA, 5 end /clone=IMAG; 2058_s_at M35011 /FEATURE= /DEFINITION=HUMIBSUB Human integrin beta-5 subunit mR; 1597_at L13720 /FEATURE= /DEFINITION=HUMGAS Homo sapiens growth-arrest-specific; 1598_g_at L13720 /FEATURE= /DEFINITION=HUMGAS Homo sapiens growth-arrest-specifi; 1447_at D00761 /FEATURE= /DEFINITION=HUMPSC5 Human mRNA for proteasome subunit H; 1314_at D44466 /FEATURE= /DEFINITION=D44466 Homo sapiens mRNA for proteasome sub; 1016_s_at U70981 /FEATURE= /DEFINITION=HSU70981 Human interleukin-13 receptor mR; 956_at Tubulin, Beta 2; 336_at D38081 /FEATURE= /DEFINITION=HUMHTAR Human mRNA for thromboxane A2 recept.

Metagene 244; 31685_at Cluster Incl. Y08976:H. sapiens mRNA for FEV protein /cds=(584,1300) /gb; 32282_at Cluster Incl. U66047:Human clone Z3-1 placenta expressed mRNA from chro; 32888_at Cluster Incl. X52213:H. sapiens Itk mRNA /cds=(258,1652) /gb=X52213 /gi=; 33463_at Cluster Incl. U39487:Human xanthine dehydrogenase/oxidase mRNA, complet; 34972_s_at Cluster Incl. AJ000522:Homo sapiens mRNA for dynein heavy chain /cds=; 35955_at Cluster Incl. S80864:cytochrome c-like polypeptide [human, lung adenoca; 37415_at Cluster Incl. AB018258:Homo sapiens mRNA for KIAA0715 protein, partial; 31818_at Cluster Incl. AI024916:ov35a06.x1 Homo sapiens cDNA, 3 end /clone=IMAG; 39445_at Cluster Incl. AF038661:Homo sapiens chromosome 1q21-1q23 beta-1,4-galac; 40507_at Cluster Incl. K03195:Human (HepG2) glucose transporter gene mRNA, compl; 32816_at Cluster Incl. AL050156:Homo sapiens mRNA; cDNA DKFZp586N1020 (from clon; 32837_at Cluster Incl. U56418:Human lysophosphatidic acid acyltransferase-beta m; 33935_at Cluster Incl. AL035305:H. sapiens gene from PAC 102G20 /cds=(117,803) /g; 39122_at Cluster Incl. K03515:Human neuroleukin mRNA, complete cds /cds=(15,1691; 39866_at Cluster Incl. AB028986:Homo sapiens mRNA for KIAA1063 protein, partial; 39899_at Cluster Incl. AC005525:Homo sapiens chromosome 19, cosmid F22162 /cds=(; 40170_at Cluster Incl. AI687306:tp86a09.x1 Homo sapiens cDNA, 3 end /clone=IMAG; 40254_s_at Cluster Incl. AI922937:wn66d06.x1 Homo sapiens cDNA, 3 end /clone=IM; 41281_s_at Cluster Incl. AF060502:Homo sapiens peroxisome assembly protein PEX10; 41331_at Cluster Incl. R93981:yt73d01.s1 Homo sapiens cDNA, 3 end /clone=IMAGE-; 41496_at Cluster Incl. AL050189:Homo sapiens mRNA; cDNA DKFZp586B0323 (from clon; 41500_at Cluster Incl. AI761818:wi62g02.x1 Homo sapiens cDNA, 3 end /clone=IMAG; 32550_r_at Cluster Incl. Y11525:H. sapiens mRNA for CCAAT/enhancer binding protei; 33134_at Cluster Incl. AB011083:Homo sapiens mRNA for KIAA0511 protein, partial; 1992_at U46922 /FEATURE= /DEFINITION=HSU46922 Human FHIT mRNA, complete cds; 1966_i_at U18334 /FEATURE=cds /DEFINITION=HSUNOSIIC1 Human nitric oxide synthase; 1760_s_at D11327 /FEATURE= /DEFINITION=HUMLCPTP Human mRNA for protein-tyrosine; 1141_at L27080 /FEATURE=cds /DEFINITION=HUMMC5R Human melanocortin 5 receptor (M; 916_at L18983 /FEATURE=mRNA /DEFINITION=HUMTYROPHO Homo sapiens tyrosine phospha; 736_f_at D87002 /FEATURE=cds#2 /DEFINITION=D87002 Homo sapiens immunoglobulin 1a; 648_at L37112 /FEATURE=mRNA /DEFINITION=HUMVVR Homo sapiens vasopressin V3 recep; 414_at X59373 /FEATURE=mRNA /DEFINITION=HSHOX4D Human HOX4D mRNA for a homeobox; 170_at U51096 /FEATURE= /DEFINITION=HSU51096 Human homeobox protein Cdx2 mRNA, c.

Metagene 245; 31499_s_at Cluster Incl. X16863:Human Fc-gamma RIII-1 cDNA for Fc-gamma receptor; 33092_at Cluster Incl. AC005946:Homo sapiens chromosome 19, cosmid R28782 /cds=(; 34609_g_at Cluster Incl. M24194:Human MHC protein homologous to chicken B comple; 32307_s_at Cluster Incl. V00503:Human mRNA encoding Pro-alpha-2 chain of type I; 32941_at Cluster Incl. M91196:Homo sapiens DNA-binding protein mRNA, complete cd; 34978_g_at Cluster Incl. AJ007395:Homo sapiens mRNA for QA79 membrane protein /c; 35474_s_at Cluster Incl. Y15915:Homo sapiens mRNA for chimaeric transcript of co; 36770_at Cluster Incl. U18671:Human Stat2 gene, complete cds /cds=(57,2612) /gb=; 38201_at Cluster Incl. U21551:Human ECA39 mRNA, complete cds /cds=(0,1154) /gb=U; 38598_at Cluster Incl. AI679353:tu73f03.x1 Homo sapiens cDNA, 3 end /clone=IMAG; 40369_f_at Cluster Incl. AL022723:dJ377H14.1 (major histocompatibility complex,; 40370_f_at Cluster Incl. M90683:Human lymphocyte antigen (HLA-G1) mRNA, complete; 34666_at Cluster Incl. X07834:Human mRNA for manganese superoxide dismutase (EC; 40519_at Cluster Incl. Y00638:Human mRNA for leukocyte common antigen (T200) /cd; 36151_at Cluster Incl. U60644:Human HU-K4 mRNA, complete cds /cds=(487,1800) /gb; 38077_at Cluster Incl. X52022:H. sapiens RNA for type VI collagen alpha3 chain /c; 2024_s_at M79321 /FEATURE= /DEFINITION=HUMLYNTK Human Lyn B protein mRNA, comple; 1991_s_at U43784 /FEATURE= /DEFINITION=HSU43784 Human mitogen activated protein; 1984_-sat X69549 /FEATURE=cds /DEFINITION=HSRHO2 H. sapiens mRNA for rho GDP-diss; 1146_at Cd4 Antigen; 1150_at Tyrosine Phosphatase, Epsilon; 834_at U40462 /FEATURE= /DEFINITION=HSU40462 Human Ikaros/LyF-1 homolog (hIk-1); 428_s_at V00567 /FEATURE=cds /DEFINITION=HSMGLO Human messenger RNA fragment for; 201_s_at S82297 /FEATURE= /DEFINITION=S82297 beta 2-microglobulin {11 bp deleted; 184_at U03642 /FEATURE=cds /DEFINITION=HSU03642 Human G protein-coupled receptor; 129_g_at X82153 /FEATURE=cds /DEFINITION=HSOC2RNA H. sapiens mRNA for cathepsin 0; 160041_at X79568 /FEATURE=cds /DEFINITION=HSBDP1 H. sapiens BDP1 mRNA for protein.

Metagene 246; 38855_s_at Cluster Incl. D82343:Homo sapiens mRNA for AMY, complete cds /cds=(28; 38957_at Cluster Incl. AB002367:Human mRNA for KIAA0369 gene, complete cds /cds=; 41665_at Cluster Incl. AB020631:Homo sapiens mRNA for KIAA0824 protein, partial; 32084_at Cluster Incl. AF057164:Homo sapiens organic cation transporter OCTN2 (O; 37983_at Cluster Incl. S77410:type 1 angiotensin II receptor [human, liver, mRNA; 40155_at Cluster Incl. D31883:Human mRNA for KIAA0059 gene, complete cds /cds=(2; 35277_at Cluster Incl. AB018305:Homo sapiens mRNA for KIAA0762 protein, partial; 37729_at Cluster Incl. Y08614:Homo sapiens mRNA for CRM1 protein /cds=(38,3253); 38026_at Cluster Incl. U01244:Human fibulin-1D mRNA, complete cds /cds=(10,2121); 39550_at Cluster Incl. AB011156:Homo sapiens mRNA for KIAA0584 protein, partial; 40239_g_at Cluster Incl. AI674208:wc07f02.x1 Homo sapiens cDNA, 3 end /clone=IM; 40240_at Cluster Incl. AC004131:Homo sapiens Chromosome 16 BAC clone CIT987SK-A-; 41839_at Cluster Incl. L13698:Human gas1 gene, complete cds /cds=(410,1447) /gb=; 962_at X83107 /FEATURE=cds /DEFINITION=HSBMXGENE H. sapiens Bmx mRNA for cytoplas; 661_at L13698 /FEATURE= /DEFINITION=HUMGAS1A Human gas1 gene, complete cds; 615_s_at M24351 /FEATURE=expanded_cds /DEFINITION=HUMPTHL4 Human parathyroid hor; 160030_at X06562 /FEATURE=cds /DEFINITION=HSGHR Human mRNA for growth hormone re.

Metagene 247; 31693_f_at Cluster Incl. Z80776:H. sapiens H2A/g gene /cds=(0,392) /gb=Z80776 /gi; 41053_at Cluster Incl. AJ245433:Homo sapiens mRNA for G4 protein (G4 gene, locat; 41636_at Cluster Incl. AB014565:Homo sapiens mRNA for KIAA0665 protein, complete; 32655_s_at Cluster Incl. X87613:H. sapiens mRNA for skeletal muscle abundant prot; 33289_f_at Cluster Incl. D88827:Homo sapiens mRNA for zinc finger protein FPM315; 36040_at Cluster Incl. AI337192:qx88h10.x1 Homo sapiens cDNA, 3 end /clone=IMAG; 38007_at Cluster Incl. L11353:Human moesin-ezrin-radixin-like protein mRNA, comp; 38260_at Cluster Incl. AL050306:Human DNA sequence from clone 475B7 on chromosom; 33350_s_at Cluster Incl. Z78315:HSZ78315 Homo sapiens cDNA /clone=2.120-(CEPH) /; 34300_at Cluster Incl. AI352450:qt16g11.x1 Homo sapiens cDNA, 3 end /clone=IMAG; 37326_at Cluster Incl. U93305:Homo sapiens A4 differentiation-dependent protein; 33165_at Cluster Incl. AL041879:DKFZp434H0419_r1 Homo sapiens cDNA, 5 end /clon; 992_at X52221 /FEATURE=mRNA /DEFINITION=HSERCC25 H. sapiens ERCC2 gene, exons 1 &.

Metagene 248; 32363_at Cluster Incl. AF059214:Homo sapiens cholesterol 25-hydroxylase mRNA, co; 39310_at Cluster Incl. X86163:H. sapiens mRNA for B2-bradykinin receptor, 3 /cds=; 40659_at Cluster Incl. U12767:Human mitogen induced nuclear orphan receptor (MIN; 40662_g_at Cluster Incl. D78579:Homo sapiens mRNA for neuron derived orphan rece; 41386_i_at Cluster Incl. AB002344:Human mRNA for KIAA0346 gene, partial cds /cds; 41387_r_at Cluster Incl. AB002344:Human mRNA for KIAA0346 gene, partial cds /cds; 37187_at Cluster Incl. M36820:Human cytokine (GRO-beta) mRNA, complete cds /cds=; 38299_at Cluster Incl. X04430:Human IFN-beta 2a mRNA for interferon-beta-2 /cds=; 39402_at Cluster Incl. M15330:Human interleukin 1-beta (IL1B) mRNA, complete cds; 41779_at Cluster Incl. U70426:Homo sapiens A28-RGS14p mRNA, complete cds /cds=(9; 32786_at Cluster Incl. X51345:Human jun-B mRNA for JUN-B protein /cds=(253,1296); 36097_at Cluster Incl. M62831:Human transcription factor ETR101 mRNA, complete c; 36979_at Cluster Incl. M20681:Human glucose transporter-like protein-III (GLUT3); 37701_at Cluster Incl. L13463:Human helix-loop-helix basic phosphoprotein (G0S8); 37724_at Cluster Incl. V00568:Human mRNA encoding the c-myc oncogene /cds=(558,1; 39143_at Cluster Incl. U08015:Human NF-ATc mRNA, complete cds /cds=(239,2389) /g; 2049_s_at M29039 /FEATURE=cds /DEFINITION=HUMJUNCAA Human transactivator Oun-B); 1973_s_at V00568 /FEATURE=cds /DEFINITION=HSMYC1 Human mRNA encoding the c-myc o; 1779_s_at M16750 /FEATURE= /DEFINITION=HUMPIM1 Human pim-1 oncogene mRNA, comple; 1520_s_at X04500 /FEATURE=expanded_cds /DEFINITION=HSIL1B Human gene for prointe; 1334_s_at X03656 /FEATURE=mRNA /DEFINITION=HSGCSFG Human gene for granulocyte co; 529_at U15932 /FEATURE= /DEFINITION=HSU15932 Human dual-specificity protein phos; 265_s_at M24736 /FEATURE= /DEFINITION=HUMELAM1A Human endothelial leukocyte adhe; 190_at U12767 /FEATURE= /DEFINITION=HSU12767 Human mitogen induced nuclear orpha.

Metagene 249; 41860_at Cluster Incl. AF070559:Homo sapiens clone 24463 mRNA sequence /cds=UNKN; 39039_s_at Cluster Incl. AI557497:Pt2.1_{—}16_A04.r Homo sapiens cDNA, 3 end /clon; 40874_at Cluster Incl. AJ005259:Homo sapiens mRNA for EDF-1 protein /cds=(34,480; 41193_at Cluster Incl. AB013382:Homo sapiens mRNA for DUSP6, complete cds /cds=(; 33821_at Cluster Incl. AL034374:Human DNA sequence from clone 483K16 on chromoso; 36578_at Cluster Incl. U37547:Human IAP homolog B (MIHB) mRNA, complete cds /cds; 37757_at Cluster Incl. L23959:Homo sapiens E2F-related transcription factor (DP-; 38374_at Cluster Incl. AF050110:Homo sapiens TGFb inducible early protein and ea; 39092_at Cluster Incl. AW007731:wt68d11.x1 Homo sapiens cDNA, 3 end /clone=IMAG; 39174_at Cluster Incl. X77548:H. sapiens cDNA for RFG /cds=(76,1920) /gb=X77548; 39181_at Cluster Incl. U00928:Human clone CE29 4.1 (CAC)n/(GTG)n repeat-containi; 1670_at L23959 /FEATURE= /DEFINITION=HUMDP1A Homo sapiens E2F-related transcript; 1466_s_at S81661 /FEATURE= /DEFINITION=S81661 Keratinocyte growth factor [human,; 843_at U48296 /FEATURE= /DEFINITION=HSU48296 Homo sapiens protein tyrosine phosp.

Metagene 250; 37877_at Cluster Incl. AL050269:Homo sapiens mRNA; cDNA DKFZp564C103 (from clone; 38594_i_at Cluster Incl. AB006622:Homo sapiens mRNA for KIAA0284 gene, partial c; 34289_f_at Cluster Incl. D50920:Human mRNA for KIAA0130 gene, complete cds /cds=; 34702_f_at Cluster Incl. M27826:Human endogenous retroviral protease mRNA, compl; 35617_at Cluster Incl. U29725:Human BMK1 alpha kinase mRNA, complete cds /cds=(2; 36005_at Cluster Incl. AF042800:Homo sapiens suppressor of white apricot homolog; 37269_at Cluster Incl. D38496:Human mRNA for LZTR-1, complete cds /cds=(862,2520; 37910_at Cluster Incl. U52112:host cell factor 1 /cds=(344,6451) /gb=U52112 /gi=; 40123_at Cluster Incl. D87435:Human mRNA for KIAA0248 gene, partial cds /cds=(0,; 32801_at Cluster Incl. AB002315:Human mRNA for KIAA0317 gene, complete cds /cds=; 33909_at Cluster Incl. L35013:Human spliceosomal protein (SAP 49) gene, complete; 34404_at Cluster Incl. W28167:43a1 Homo sapiens cDNA /gb=W28167 /gi=1308115 /ug=; 35771_at Cluster Incl. AF049460:Homo sapiens nuclear DEAF-1 related transcriptio; 36940_at Cluster Incl. D86970:Human mRNA for KIAA0216 gene, complete cds /cds=(4; 38043_at Cluster Incl. X55448:H. sapiens G6PD gene for glucose-6-phosphate dehydr; 38829_r_at Cluster Incl. AA628946:af28f05.s1 Homo sapiens cDNA, 3 end /clone=IM; 40263_at Cluster Incl. AF001891:Homo sapiens clone lambda MEN1 region unknown pr; 41836_at Cluster Incl. U94836:Human ERPROT 213-21 mRNA, complete cds /cds=(88,27; 1181_at Guanine Nucleotide-Binding Protein Hsr1; 1162_at Guanine Nucleotide-Binding Protein Hsr1.

Metagene 251; 31688_at Cluster Incl. AF005080:Homo sapiens skin-specific protein (xp5) mRNA, c; 35022_at Cluster Incl. S83308:SOX5=Sry-related HMG box gene {alternatively splic; 39615_at Cluster Incl. AB028949:Homo sapiens mRNA for KIAA1026 protein, partial; 35217_at Cluster Incl. AL049404:Homo sapiens mRNA; cDNA DKFZp586F0219 (from clon; 41345_at Cluster Incl. AI223140:qg53a06.x1 Homo sapiens cDNA, 3 end /clone=IMAG; 514_at U26710 /FEATURE= /DEFINITION=HSU26710 Human cbl-b mRNA, complete cds.

Metagene 252; 34636_at Cluster Incl. M23892:Human 15-lipoxygenase mRNA, complete cds /cds=(3,1; 38932_at Cluster Incl. M29580:Human zinc-finger protein 7 (ZFP7) mRNA, complete; 39638_at Cluster Incl. S73885:AP-4=basic helix-loop-helix DNA-binding protein [h; 41639_at Cluster Incl. D38553:Human mRNA for KIAA0074 gene, partial cds /cds=(0,; 35145_at Cluster Incl. X96401:H. sapiens mRNA for ROX protein /cds=(212,1960) /gb.

Metagene 253; 36264_at Cluster Incl. S75168:Matk-megakaryocyte-associated tyrosine kinase [hum; 37149_s_at Cluster Incl. U95626:Homo sapiens ccr2b (ccr2), ccr2a (ccr2), ccr5 (c; 41063_g_at Cluster Incl. AA037278:zc52c04.r1 Homo sapiens cDNA, 5 end /clone=IM; 32560_s_at Cluster Incl. W30959:zc65h10.r1 Homo sapiens cDNA, 5 end /clone=IMAG.

Metagene 254; 31413_at Cluster Incl. AF000990:Homo sapiens testis-specific Testis Transcript Y; 32428_at Cluster Incl. AF026564:Homo sapiens RNA binding protein II (RBMII) gene; 32924_at Cluster Incl. AB021227:Homo sapiens mRNA for membrane-type-5 matrix met; 34522_at Cluster Incl. AF070625:Homo sapiens clone 24734 mRNA sequence /cds=UNKN; 38158_at Cluster Incl. D79987:Human mRNA for KIAA0165 gene, complete cds /cds=(1; 33784_at Cluster Incl. U12597:Human tumor necrosis factor type 2 receptor associ; 1004_at X68149 /FEATURE=exon#2 /DEFINITION=HSBLR1A Homo sapiens BLR1 gene for Bu.

Metagene 255; 31510_s_at Cluster Incl. Z48950:H. sapiens hH3.3B gene for histone H3.3 /cds=(10,; 36347_f_at Cluster Incl. AA873858:oh79b10.s1 Homo sapiens cDNA, 3 end /clone=IM; 33226_at Cluster Incl. AB020683:Homo sapiens mRNA for KIAA0876 protein, partial; 35672_at Cluster Incl. AL080144:Homo sapiens mRNA; cDNA DKFZp434N093 (from clone; 39435_at Cluster Incl. D45333:HUMHG7879 Homo sapiens cDNA /gb=D45333 /gi=1136736; 41183_at Cluster Incl. U15782:Human cleavage stimulation factor 77 kDa subunit mR; 842_at U48251 /FEATURE= /DEFINITION=HSU48251 Homo sapiens protein kinase C-bindi.

Metagene 256; 33997_at Cluster Incl. AL049449:Homo sapiens mRNA; cDNA DKFZp586B1722 (from clon; 34981_at Cluster Incl. M55513:Human potassium channel (HPCN1) mRNA, complete cds; 41430_at Cluster Incl. AB011113:Homo sapiens mRNA for KIAA0541 protein, partial; 41463_at Cluster Incl. AL042729:DKFZp434B0222_s1 Homo sapiens cDNA, 3 end /clon; 32686_at Cluster Incl. D86096:Human DNA for prostagrandin E receptor EP3 subtype; 33787_at Cluster Incl. AB011109:Homo sapiens mRNA for KIAA0537 protein, complete; 35168_f_at Cluster Incl. M92642:Homo sapiens alpha-1 type XVI collagen (COL16A1); 37259_at Cluster Incl. Z81326:H. sapiens mRNA for protease inhibitor 12 (PI12; ne; 37279_at Cluster Incl. U10550:Human Gem GTPase (gem) mRNA, complete cds /cds=(21; 37602_at Cluster Incl. Z49878:H. sapiens mRNA for guanidinoacetate N-methyltransf; 38711_at Cluster Incl. AB014527:Homo sapiens mRNA for KIAA0627 protein, partial; 39754_at Cluster Incl. X53002:Human mRNA for integrin beta-5 subunit /cds=(336,2; 40446_at Cluster Incl. AL021366:cICK0721Q.4.1 (PHD finger protein 2) (isoform 2); 41742_s_at Cluster Incl. AF061034:Homo sapiens FIP2 alternatively translated mRN; 33358_at Cluster Incl. W29087:56b8 Homo sapiens cDNA /gb=W29087 /gi=1309053 /ug=; 33380_at Cluster Incl. AB005754:Homo sapiens mRNA for LAK-1, complete cds /cds=(; 33408_at Cluster Incl. AB023151:Homo sapiens mRNA for KIAA0934 protein, partial; 37338_at Cluster Incl. D61391:Human mRNA for phosphoribosypyrophosphate syntheta; 37406_at Cluster Incl. X94232:H. sapiens mRNA for novel T-cell activation protein; 38431_at Cluster Incl. U09759:Human protein kinase (JNK2) mRNA, complete cds /cd; 38803_at Cluster Incl. AF052142:Homo sapiens clone 24665 mRNA sequence /cds=UNKN; 33169_at Cluster Incl. U61262:Human neogenin mRNA, complete cds /cds=(136,4521); 1861_at U66879 /FEATURE= /DEFINITION=HSU66879 Human Bcl-2 binding component 6 (b.

Metagene 257; 31742_at Cluster Incl. AF064090:Homo sapiens ligand for herpesvirus entry mediat; 32135_at Cluster Incl. U00968:Human SREBP-1 mRNA, complete cds /cds=(166,3609) /; 38672_at Cluster Incl. Y13247:Homo sapiens fb19 mRNA /cds=(539,3361) /gb=Y13247; 32783_at Cluster Incl. X82494:H. sapiens mRNA for fibulin-2 /cds=(69,3623) /gb=X8; 36933_at Cluster Incl. D87953:Human mRNA for RTP, complete cds /cds=(122,1306) /; 37761_at Cluster Incl. AB015020:Homo sapiens mRNA for BAP2-beta protein, complet; 1563_s_at M58286 /FEATURE= /DEFINITION=HUMTNFRB Homo sapiens tumor necrosis fact; 675_at J04164 /FEATURE= /DEFINITION=HUM927A Human interferon-inducible protein 9; 195_s_at U28014 /FEATURE= /DEFINITION=HSU28014 Human cysteine protease (ICErel-I.

Metagene 258; 34239_at Cluster Incl. AL049787:Novel human gene mapping to chomosome 13 /cds=(5; 38634_at Cluster Incl. M11433:Human cellular retinol-binding protein mRNA, compl; 39710_at Cluster Incl. U30521:Human P311 HUM (3.1) mRNA, complete cds /cds=(202,; 34296_at Cluster Incl. AF041210:Homo sapiens midline 1 fetal kidney isoform 3 (M; 37657_at Cluster Incl. Y16270:Homo sapiens PALM gene, exon 1 and joined CDS /cds; 38072_at Cluster Incl. AL031432:Human DNA sequence from clone 465N24 on chromoso; 38479_at Cluster Incl. Y07969:H. sapiens mRNA for APRIL protein /cds=(230,979) /g; 1975_s_at X03563 /FEATURE=cds /DEFINITION=HSIGF1G1 Human gene for insulin-like g; 1761_at D37965 /FEATURE= /DEFINITION=HUMPRLTS Human mRNA for PDGF receptor beta-; 1709_g_at U07620 /FEATURE= /DEFINITION=HSU07620 Human MAP kinase mRNA, complete; 1246_at U35234 /FEATURE= /DEFINITION=HSU35234 Human protein tyrosine phosphatase.

Metagene 259; 39610_at Cluster Incl. X16665:Human HOX2H m-RNA from the Hox2 locus /cds=(78,1148; 36091_at Cluster Incl. AF051323:Homo sapiens Src-associated adaptor protein (SAP; 37618_at Cluster Incl. M16937:Human homeo box c1 protein, mRNA, complete cds /cd; 38294_at Cluster Incl. X17360:Human HOX 5.1 gene for HOX 5.1 protein /cds=(1243,; 37015_at Cluster Incl. K03000:Human aldehyde dehydrogenase 1 mRNA /cds=(0,1022); 39840_at Cluster Incl. AI037867:oy02f10.x1 Homo sapiens cDNA, 3 end /clone=IMAG; 33203_s_at Cluster Incl. U59831:Human transcription factor, forkhead related act; 873_at M26679 /FEATURE=expanded_cds /DEFINITION=HUMHOX13G Homo sapiens homeobox; 444_g_at X17360 /FEATURE=mRNA /DEFINITION=HSHOX51 Human HOX 5.1 gene for HOX 5.1.

Metagene 260; 31544_at Cluster Incl. L13203:Human HNF-3 /fork-head homolog-3 HFH-3 mRNA, comple; 35045_r_at Cluster Incl. X60655:H. sapiens EVX1 mRNA /cds=(228,1451) /gb=X60655 /; 36016_at Cluster Incl. AF013252:Homo sapiens preprocortistatin (Cort) mRNA, comp; 1092_at M65199 /FEATURE= /DEFINITION=HUMET2A Human endothelin 2 (ET2) mRNA, comp.

Metagene 261; 32001_s_at Cluster Incl. M80482:Human subtilisin-like protein (PACE4) mRNA, comp; 36412_s_at Cluster Incl. U53831:Homo sapiens interferon regulatory factor 7B mRN; 34974_at Cluster Incl. Y13323:Homo sapiens mRNA for disintegrin-protease /cds=(6; 41038_at Cluster Incl. M32011:Human neutrophil oxidase factor (p67-phox) mRNA, c; 41106_at Cluster Incl. AF022797:Homo sapiens intermediate conductance calcium-ac; 41409_at Cluster Incl. AF044896:Homo sapiens ICB-1 mRNA, complete cds /cds=(128,; 41678_at Cluster Incl. AF025304:Homo sapiens protein-tyrosine kinase EPHB2v (EPH; 31891_at Cluster Incl. U58515:Human chitinase (HUMTCHIT) mRNA, exon 1b form, par; 33227_at Cluster Incl. AI984234:wz57e04.x1 Homo sapiens cDNA, 3 end /clone=IMAG; 33731_at Cluster Incl. AJ130718:Homo sapiens mRNA for glycoprotein-associated am; 33752_at Cluster Incl. AB020657:Homo sapiens mRNA for KIAA0850 protein, complete; 34749_at Cluster Incl. U83461:Human putative copper uptake protein (hCTR2) mRNA,; 37542_at Cluster Incl. D86961:Human mRNA for KIAA0206 gene, partial cds /cds=(0,; 37922_at Cluster Incl. L02648:Homo sapiens (clone V6) transcobalamin II (TCN2) m; 39774_at Cluster Incl. X80695:H. sapiens OXA1Hs mRNA /cds=(6,1313) /gb=X80695 /gi; 40049_at Cluster Incl. X76104:H. sapiens DAP-kinase mRNA /cds=(336,4631) /gb=X761; 40432_at Cluster Incl. AA522891:ni41b12.s1 Homo sapiens cDNA, 3 end /clone=IMAG; 41209_at Cluster Incl. M15856:Human lipoprotein lipase mRNA, complete cds /cds=U; 36963_at Cluster Incl. U30255:Human phosphogluconate dehydrogenase (hPGDH) gene,; 37021_at Cluster Incl. X116832:Human mRNA for cathepsin H (EC 3.4.22.16) /cds=(34; 37025_at Cluster Incl. AL120815:DKFZp762F172_r1 Homo sapiens cDNA, 5 end /clone; 37343_at Cluster Incl. U01062:Human type 3 inositol 1,4,5-trisphosphate receptor; 1647_at U51903 /FEATURE= /DEFINITION=HSU51903 Human RasGAP-related protein (IQGA; 1085_s_at M37238 /FEATURE=mRNA /DEFINITION=HUMPLC Human phospholipase C mRNA, co.

Metagene 262; 31974_at Cluster Incl. AJ132445:Homo sapiens CLDN14 gene /cds=(0,719) /gb=AJ1324; 33124_at Cluster Incl. AB000450:Homo sapiens mRNA for VRK2, complete cds /cds=(1; 1591_s_at J03242 /FEATURE= /DEFINITION=HUMGFIL2 Human insulin-Ike growth factor.

Metagene 263; 37578_at Cluster Incl. D25248:Homo sapiens mRNA, clone-RES4-4 /cds=UNKNOWN /gb=D; 37584_at Cluster Incl. AJ007669:Homo sapiens mRNA for Fanconi anemia group G /cd; 34782_at Cluster Incl. AL021938:Homo sapiens DNA sequence from PAC 232K4 on chro; 37037_at Cluster Incl. M24486:Human prolyl 4-hydroxylase alpha subunit mRNA, com; 37295_at Cluster Incl. X63469:H. sapiens mRNA for transcription factor TFIIE beta; 39468_r_at Cluster Incl. W27081:22g5 Homo sapiens cDNA /gb=W27081 /gi=1306660 /u; 41264_at Cluster Incl. AL050172:Homo sapiens mRNA; cDNA DKFZp586F1322 (from clon; 2088_-sat D31661 /FEATURE= /DEFINITION=HUMERKA Human mRNA for tyrosine kinase, c; 1767_s_at X14885 /FEATURE=mRNA /DEFINITION=HSTGF31 H. sapiens gene for transformi; 1711_at U09477 /FEATURE= /DEFINITION=HSU09477 Human clone 53BP1 p53-binding prot; 1688_s_at X87843 /FEATURE=cds /DEFINITION=HSCYCHASS H. sapiens mRNA for cyclin H; 1374_g_at M31523 /FEATURE= /DEFINITION=HUMTFAA Human transcription factor (E2A); 224_at S81439 /FEATURE= /DEFINITION=S81439 EGR alpha=early growth response gene.

Metagene 264; 36396_at Cluster Incl. AL049443:Homo sapiens mRNA; cDNA DKFZp586N2020 (from clon; 37816_at Cluster Incl. M57729:Human complement component CS mRNA, complete cds /; 40704_at Cluster Incl. Z29090:H. sapiens mRNA for phosphatidylinositol 3-kinase /; 32676_at Cluster Incl. M93405:Human methylmalonate semialdehyde dehydrogenase ge; 32697_at Cluster Incl. AF042729:Homo sapiens lithium-sensitive myo-inositol mono; 34261_at Cluster Incl. D84307:Homo sapiens mRNA for phosphoethanolamine cytidyly; 35987_g_at Cluster Incl. AL050395:Homo sapiens mRNA; cDNA DKFZp586D1020 (from cl; 36860_at Cluster Incl. AB028987:Homo sapiens mRNA for KIAA1064 protein, partial; 37249_at Cluster Incl. AF079529:Homo sapiens cAMP-specific phosphodiesterase 8B; 37251_s_at Cluster Incl. AF016004:Homo sapiens m6b1 mRNA, complete cds /cds=(254; 38351_at Cluster Incl. AL050154:Homo sapiens mRNA; cDNA DKFZp586L0120 (from clon; 39377_at Cluster Incl. D87453:Human mRNA for KIAA0264 gene, partial cds /cds=(0,; 39422_at Cluster Incl. AB007923:Homo sapiens mRNA for KIAA0454 protein, partial; 39746_at Cluster Incl. X63563:H. sapiens mRNA for RNA polymerase II 140 kDa subun; 40161_at Cluster Incl. L32137:Human germline oligomeric matrix protein (COMP) mR; 40834_at Cluster Incl. AB002298:Human mRNA for KIAA0300 gene, partial cds /cds=(; 32253_at Cluster Incl. AB007927:Homo sapiens mRNA for KIAA0458 protein, complete; 33446_at Cluster Incl. W26407:29b8 Homo sapiens cDNA /gb=W26407 /gi=1307106 /ug=; 34372_at Cluster Incl. AB002310:Human mRNA for KIAA0312 gene, partial cds /cds=(; 35342_at Cluster Incl. AF052159:Homo sapiens clone 24416 mRNA sequence /cds=UNKN; 35795_at Cluster Incl. AJ011972:Homo sapiens mRNA for histone deacetylase-like p; 38105_at Cluster Incl. W26521:32g11 Homo sapiens cDNA /gb=W26521 /gi=1307382 /ug; 41249_at Cluster Incl. AL031282:dJ283E3.6.1 (PUTATIVE novel protein similar to m; 1171_s_at Transcription Factor Oct-1a/1b, Alt. Splice 2, Oct-1b; 1058_at S69790 /FEATURE= /DEFINITION=S69790 Brush-1=tumor suppressor {3 region}; 819_at U76456 /FEATURE= /DEFINITION=HSU76456 Homo sapiens tissue inhibitor of me; 218_at S74221 /FEATURE= /DEFINITION=S74221 IK=IK factor [human, leukemic cells K.

Metagene 265; 38923_at Cluster Incl. L76159:Homo sapiens FRG1 mRNA, complete cds /cds=(191,967; 40036_at Cluster Incl. AF035940:Homo sapiens MAGOH mRNA, complete cds /cds=(65,5; 31822_at Cluster Incl. L12579:Human alternatively spliced CUTL1 mRNA, complete c; 35218_at Cluster Incl. AF022385:Homo sapiens apoptosis-related protein TFAR15 (T; 35224_at Cluster Incl. AF070569:Homo sapiens clone 24659 mRNA sequence /cds=UNKN; 36013_at Cluster Incl. AF006621:Homo sapiens embryonic lung protein (HUEL) mRNA,; 39743_at Cluster Incl. AI862521:wj15a06.x1 Homo sapiens cDNA, 3 end /clone=IMAG; 39767_at Cluster Incl. D13627:Human mRNA for KIAA0002 gene, complete cds /cds=(2; 40076_at Cluster Incl. AF004430:Homo sapiens hD54+ins2 isoform (hD54) mRNA, comp; 40410_at Cluster Incl. W26651:34c5 Homo sapiens cDNA /gb=W26651 /gi=1307494 /ug=; 33343_at Cluster Incl. AB022663:Homo sapiens HFB30 mRNA, complete cds /cds=(236,; 34305_at Cluster Incl. Z29505:H. sapiens mRNA for nucleic acid binding protein su; 34893_at Cluster Incl. AI557064:PT2.1_{—}13_A12.r Homo sapiens cDNA, 3 end /clone_{—}; 35349_at Cluster Incl. AF031647:Homo sapiens JAB I-containing signalosome subunit; 35744_at Cluster Incl. D50931:Human mRNA for KIAA0141 gene, complete cds /cds=(8; 36122_at Cluster Incl. X59417:H. sapiens PROS-27 mRNA /cds=(62,802) /gb=X59417 /g; 36687_at Cluster Incl. N50520:yy89b05.s1 Homo sapiens cDNA, 3 end /clone=IMAGE-; 37381_g_at Cluster Incl. X59268:Human mRNA for general transcription factor IIB; 37403_at Cluster Incl. X05908:Human mRNA for lipocortin /cds=(74,1114) /gb=X0590; 37659_at Cluster Incl. L42572:Homo sapiens p87/89 gene, complete cds /cds=(92,23; 38068_at Cluster Incl. M63175:Human autocrine motility factor receptor mRNA /cds; 38399_at Cluster Incl. AL034428:Human DNA sequence from clone 705D16 on chromoso; 38450_at Cluster Incl. X69804:H. sapiens mRNA for La/SS-B protein /cds=UNKNOWN /g; 38744_at Cluster Incl. N95406:zb80g12.s1 Homo sapiens cDNA, 3 end /clone=IMAGE-; 38801_at Cluster Incl. AI742846:wg46h09.x1 Homo sapiens cDNA, 3 end /clone=IMAG; 40903_at Cluster Incl. AL049929:Homo sapiens mRNA; cDNA DKFZp547O0510 (from clon; 41510_s_at Cluster Incl. L15189:Homo sapiens mitochondrial HSP75 mRNA, complete; 1825_at L33075 /FEATURE= /DEFINITION=HUMIQGA Homo sapiens ras GTPase-activating-; 1399_at L34587 /FEATURE= /DEFINITION=HUMRPIE Homo sapiens RNA polymerase II elon; 924_s_at J03805 /FEATURE= /DEFINITION=HUMALPHLB Human phosphatase 2A mRNA, parti; 781_at X98001 /FEATURE=cds /DEFINITION=HSGGII H. sapiens mRNA for geranylgeranyl.

Metagene 266; 34022_at Cluster Incl. M36821:Human cytokine (GRO-gamma) mRNA, complete cds /cds; 40672_at Cluster Incl. U57721:Human L-kynurenine hydrolase mRNA, complete cds /c; 32640_at Cluster Incl. M24283:Human major group rhinovirus receptor (HRV) mRNA,; 38631_at Cluster Incl. M92357:Homo sapiens B94 protein mRNA, complete cds /cds=(; 1369_s_at M28130 /FEATURE=mRNA /DEFINITION=HUMIL8A Human interleukin 8 (IL8) gen; 595_at M59465 /FEATURE= /DEFINITION=HUMA20 Human tumor necrosis factor alpha ind; 408_at X54489 /FEATURE=mRNA /DEFINITION=HSMGSAG Human gene for melanoma growth s.

Metagene 267; 37163_at Cluster Incl. AL050374:Homo sapiens mRNA; cDNA DKFZp586C1619 (from clon; 33770_at Cluster Incl. AF009225:Homo sapiens IkB kinase alpha subunit (IKK alpha; 35238_at Cluster Incl. AB000509:Homo sapiens mRNA for TRAF5, complete cds /cds=(; 36505_at Cluster Incl. D55655:Homo sapiens mRNA for cardiac calsequestrin, compl; 37254_at Cluster Incl. U09366:Human zinc finger protein ZNF133 /cds=(445,2409) /; 40080_at Cluster Incl. D42044:Human mRNA for KIAA0090 gene, partial cds /cds=(0,; 32858_at Cluster Incl. AI341565:qq94g11.x1 Homo sapiens cDNA, 3 end /clone=IMAG; 33392_at Cluster Incl. AL080155:Homo sapiens mRNA; cDNA DKFZp434J154 (from clone; 35297_at Cluster Incl. AC002400:Human Chromosome 16 BAC clone CIT987SK-A-735G6 /; 35765_at Cluster Incl. X91504:H. sapiens mRNA for ARP1 protein /cds=(11,616) /gb=; 36676_at Cluster Incl. AL031659:dJ343K2.2.1 (ribophorin II (isoform 1)) /cds=(28; 38115_at Cluster Incl. AF055479:Homo sapiens lung cancer candidate FUS1 (FUS1) m; 38456_s_at Cluster Incl. AL049650:dJ734P14.2.2 (snRNP (small nuclear ribonucleop; 38484_at Cluster Incl. D21267:Homo sapiens mRNA, complete cds /cds=(205,825) /gb; 40593_at Cluster Incl. X66975:H. sapiens mRNA for heterogeneous nuclear ribonucle; 549_at S80343 /FEATURE= /DEFINITION=S80343 ArgRS=arginyl-tRNA synthetase [human,.

Metagene 268; 31451_at Cluster Incl. U62794:Human CDC42 GAP-related protein mRNA, partial cds; 31536_at Cluster Incl. AB020693:Homo sapiens mRNA for KIAA0886 protein, complete; 34642_at Cluster Incl. U28964:Homo sapiens 14-3-3 protein mRNA, complete cds /cd; 33989_f_at Cluster Incl. W28869:53h2 Homo sapiens cDNA /gb=W28869 /gi=1308880 /u; 34953_i_at Cluster Incl. D89094:Homo sapiens mRNA for 3,5-cyclic GMP phosphodie; 35968_s_at Cluster Incl. AF001307:Homo sapiens aryl hydrocarbon receptor nuclear; 36262_at Cluster Incl. Z12173:H. sapiens GNS mRNA encoding glucosamine-6-sulphata; 36263_g_at Cluster Incl. Z12173:H. sapiens GNS mRNA encoding glucosamine-6-sulpha; 37488_at Cluster Incl. L00635:Human farnesyl-protein transferase beta-subunit mR; 38526_at Cluster Incl. U02882:Human rolipram-sensitive 3,5-cyclic AMP phosphodi; 39277_at Cluster Incl. U60805:Human oncostatin-M specific receptor beta subunit; 39933_at Cluster Incl. X93921:H. sapiens mRNA for protein-tyrosine-phosphatase (t; 40696_at Cluster Incl. U50062:Homo sapiens RIP protein kinase mRNA, complete cds; 41061_at Cluster Incl. AF052288:untitled /cds=(0,2988) /gb=AF052288 /gi=3510692; 31829_r_at Cluster Incl. AF027515:Homo sapiens trans-golgi network glycoprotein; 33801_at Cluster Incl. AF104222:Homo sapiens CD2 cytoplasmic domain binding prot; 34691_f_at Cluster Incl. AF006087:Homo sapiens Arp2/3 protein complex subunit p2; 40126_at Cluster Incl. Z97200:Homo sapiens DNA sequence from PAC 79C4 on chromos; 40433_at Cluster Incl. W25921:15a11 Homo sapiens cDNA /gb=W25921 /gi=1306044 /ug; 40493_at Cluster Incl. L05424:Human hyaluronate receptor (CD44) gene /cds=(136,2; 33823_at Cluster Incl. D12676:Human mRNA for lysosomal sialoglycoprotein, comple; 34389_at Cluster Incl. M64108:Humanudulin 1 mRNA, 3 end /cds=(0,2531) /gb=M641; 34890_at Cluster Incl. L09235:Human vacuolar ATPase (isoform VA68) mRNA, complet; 35737_at Cluster Incl. U90549:Human non-histone chromosomal protein (NHC) mRNA,; 35806_at Cluster Incl. W26854:17b4 Homo sapiens cDNA /gb=W26854 /gi=1306217 /ug=; 38401_s_at Cluster Incl. W27594:34h4 Homo sapiens cDNA /gb=W27594 /gi=1307542 /u; 39901_at Cluster Incl. U70312:Homo sapiens integrin binding protein Del-1 (Dell); 40608_at Cluster Incl. AA013087:ze27c09.r1 Homo sapiens cDNA, 5 end /clone=IMAG; 40962_s_at Cluster Incl. D26155:Human mRNA for transcriptional activator hSNF2a,; 41300_s_at Cluster Incl. AA477898:zu34f08.r1 Homo sapiens cDNA, 5 end /clone=IM; 32571_at Cluster Incl. X68836:H. sapiens mRNA for S-adenosylmethionine synthetase; 1903_at Ras-Related Protein Rap1b; 1870_at D13540 /FEATURE= /DEFINITION=HUMSHPTP3 Homo sapiens SH-PTP3 mRNA for pro; 1694_s_at D13413 /FEATURE=mRNA /DEFINITION=HUMTA120 Human mRNA for tumor-associa; 1656_s_at U07563 /FEATURE=cds#1 /DEFINITION=HSABLGR3 Human proto-oncogene tyrosi; 1648_at U60805 /FEATURE= /DEFINITION=HSU60805 Human oncostatin-M specific recept; 953_g_at Fk506-Binding Protein, Alt. Splice 2; 757_at D28364 /FEATURE= /DEFINITION=HUMAI23 Human mRNA for annexin II, 5 UTR (se; 600_at M28215 /FEATURE= /DEFINITION=HUMRAB5A Homo sapiens GTP-binding protein (R; 370_at Z35102 /FEATURE=cds /DEFINITION=HSPROKINX H. sapiens mRNA for Ndr protein; 351_f_at D28423 /FEATURE= /DEFINITION=HUMPSF82 Human mRNA for pre-mRNA splicing; 289_at L29277 /FEATURE= /DEFINITION=HUMAPRF Homo sapiens DNA-binding protein (AP.

Metagene 269; 33494_at Cluster Incl. S69232:electron transfer flavoprotein-ubiquinone oxidored; 37452_at Cluster Incl. M15881:Human uromodulin (Tamm-Horsfall glycoprotein) mRNA; 37520_at Cluster Incl. AJ006591:Homo sapiens mRNA for cysteine-rich protein /cds; 41040_at Cluster Incl. U77664:Human RNaseP protein p38 (RPP38) mRNA, complete cd; 41439_at Cluster Incl. AJ001381:Homo sapiens incomplete cDNA for a mutated allel; 31866_at Cluster Incl. AC005239:Homo sapiens chromosome 19, cosmid F23149 /cds=(; 34253_at Cluster Incl. D83781:Human mRNA for KIAA0197 gene, partial cds /cds=(0,; 35166_at Cluster Incl. D87343:Homo sapiens mRNA for DCRA, complete cds /cds=(239; 36090_at Cluster Incl. AL080162:Homo sapiens mRNA; cDNA DKFZp434N024 (from clone; 37569_at Cluster Incl. AF035606:Homo sapiens calcium binding protein (ALG-2) mRN; 37642_at Cluster Incl. D63877:Human mRNA for KIAA0157 gene, partial cds /cds=(0,; 37991_at Cluster Incl. L38961:Human putative transmembrane protein precursor (B5; 38989_at Cluster Incl. AF035296:Homo sapiens clone 23837 mRNA sequence /cds=UNKN; 32262_at Cluster Incl. AL049669:Human gene from PAC 612B18, chromosome 1 /cds=(2; 34369_at Cluster Incl. D86987:Homo sapiens mRNA for KIAA0214 protein, complete c; 34787_at Cluster Incl. X93209:H. sapiens mRNA for NRD1 convertase /cds=UNKNOWN /g; 35329_at Cluster Incl. AF091084:Homo sapiens clone 638 unknown mRNA, complete se; 36177_at Cluster Incl. X78627:H. sapiens mRNA for translin /cds=(81,767) /gb=X786; 36597_at Cluster Incl. D21262:Human mRNA for KIAA0035 gene, partial cds /cds=(0,; 36603_at Cluster Incl. D86973:Human mRNA for KIAA0219 gene, partial cds /cds=(0,; 36612_at Cluster Incl. D87470:Human mRNA for KIAA0280 gene, partial cds /cds=(0,; 38372_at Cluster Incl. U66042:Human clone 191B7 placenta expressed mRNA from chr; 38773_at Cluster Incl. AB003151:Homo sapiens DNA, chromosome 21q22.2, PAC clone; 39116_at Cluster Incl. AF070626:Homo sapiens clone 24483 unknown mRNA, parital c; 39884_g_at Cluster Incl. AF091078:Homo sapiens clone 559 unknown mRNA, complete; 40224_s_at Cluster Incl. AB014585:Homo sapiens mRNA for KIAA0685 protein, comple; 40629_at Cluster Incl. L19783:Human GPI-H mRNA, complete cds /cds=(60,626) /gb=L; 41242_at Cluster Incl. AB011004:Homo sapiens HuUAP1 mRNA for UDP-N-acetylglucosa; 41278_at Cluster Incl. AF041474:Homo sapiens BAF53a (BAF53a) mRNA, complete cds; 41595_at Cluster Incl. AB023164:Homo sapiens mRNA for KIAA0947 protein, partial; 32595_at Cluster Incl. U07231:Homo sapiens G-rich sequence factor-1 (GRSF-1) mRN; 1371_s_at M29874 /FEATURE= /DEFINITION=HUMCYP2BB Human cytochrome P450-IIB (hIIB; 155_s_at U61397 /FEATURE= /DEFINITION=HSU61397 Human ubiquitin-homology domain p.

Metagene 270; 34003_at Cluster Incl. U47924:Human chromosome 12p13 sequence /cds=(373,1122) /g; 37466_at Cluster Incl. D84488:Homo sapiens mRNA for small GTP-binding protein, c; 41037_at Cluster Incl. U63824:Human transcription factor RTEF-1 (RTEF1) mRNA, co; 31886_at Cluster Incl. X55740:Human placental cDNA coding for 5nucleotidase (EC; 34177_at Cluster Incl. AF038660:Homo sapiens chromosome 1p33-p34 beta-1,4-galact; 34230_r_at Cluster Incl. D84454:Human mRNA for UDP-galactose translocator, compl; 34272_at Cluster Incl. U27768:Human RGP4 mRNA, complete cds /cds=(97,714) /gb=U2; 35275_at Cluster Incl. AL050025:Homo sapiens mRNA; cDNA DKFZp564D066 (from clone; 35832_at Cluster Incl. AB029000:Homo sapiens mRNA for KIAA1077 protein, partial; 36188_at Cluster Incl. D32257:Human GTF3A mRNA for Xenopus transcription factor; 36582_g_at Cluster Incl. U09510:Human glycyl-tRNA synthetase mRNA, complete cds; 36965_at Cluster Incl. U13616:Human ankyrin G (ANK-3) mRNA, complete cds /cds=(1; 37048_at Cluster Incl. U58970:Human putative outer mitochondrial membrane 34 kDa; 38422_s_at Cluster Incl. U29332:Homo sapiens heart protein (FHL-2) mRNA, complet; 38435_at Cluster Incl. U25182:Human antioxidant enzyme AOE37-2 mRNA, complete cd; 38783_at Cluster Incl. J05581:Human polymorphic epithelial mucin (PEM) mRNA, com; 40237_at Cluster Incl. AF035444:Homo sapiens p17-Beckwith-Wiedemann region 1 C (; 41531_at Cluster Incl. AI445461:tj34g07.x1 Homo sapiens cDNA, 3 end /clone=IMAG; 33213_g_at Cluster Incl. AF006751:Homo sapiens ES/130 mRNA, complete cds /cds=(7; 700_s_at Mucin 1, Epithelial, Alt. Splice 9; 658_at L12350 /FEATURE=mRNA /DEFINITION=HUMTHRSPO Human thrombospondin 2 (THBS2); 659_g_at L12350 /FEATURE=mRNA /DEFINITION=HUMTHRSPO Human thrombospondin 2 (THBS.

Metagene 271; 31700_at Cluster Incl. AF027957:Homo sapiens G protein-coupled receptor (GPR35); 31719_at Cluster Incl. X02761:Human mRNA for fibronectin (FN precursor) /cds=(0,; 34060_g_at Cluster Incl. AA586695:nn42h06.s1 Homo sapiens cDNA, 3 end /clone=IM; 34147_g_at Cluster Incl. AB019529:Homo sapiens mRNA for OGG1 protein type 2c, pa; 32269_at Cluster Incl. W26805:13a12 Homo sapiens cDNA /gb=W26805 /gi=1305889 /ug; 32352_at Cluster Incl. X52730:Human gene for phenylethanolamine N-methylase (PNM; 34415_at Cluster Incl. Z22536:Homo sapiens ALK-4 mRNA, complete CDS /cds=(0,1517; 34469_at Cluster Incl. X84746:H. sapiens Histo-blood group AB0 gene, exon 1 /cds=; 35910_f_at Cluster Incl. AJ003147:Homo sapiens complete genomic sequence between; 36218_g_at Cluster Incl. Z35102:H. sapiens mRNA for Ndr protein kinase /cds=(595,; 37475_at Cluster Incl. AC004144:Homo sapiens chromosome 19, overlapping cosmids; 37853_at Cluster Incl. AI857458:wl57e02.x1 Homo sapiens cDNA, 3 end /clone=IMAG; 37885_at Cluster Incl. AF038169:Homo sapiens clone 23790 unknown protein mRNA, c; 38530_at Cluster Incl. W26021:18c10 Homo sapiens cDNA /gb=W26021 /gi=1306306 /ug; 39281_at Cluster Incl. AB002378:Human mRNA for KIAA0380 gene, complete cds /cds=; 40299_at Cluster Incl. AF091890:Homo sapiens G-protein coupled receptor RE2 mRNA; 40359_at Cluster Incl. M91083:Human DNA-binding protein (HRC1) mRNA, complete cd; 41074_at Cluster Incl. AF062006:Homo sapiens orphan G protein-coupled receptor H; 31846_at Cluster Incl. AW003733:ws16b04.x1 Homo sapiens cDNA, 3 end /clone=IMAG; 33794_g_at Cluster Incl. U19345:Homo sapiens AR1 (TCF20) mRNA, partial cds /cds=; 35986_at Cluster Incl. AL050395:Homo sapiens mRNA; cDNA DKFZp586D1020 (from clon; 35996_at Cluster Incl. X98261:H. sapiens mRNA for M-phase phosphoprotein, mpp5 /c; 37285_at Cluster Incl. X60364:Human ALAS mRNA for 5-aminolevulinate synthase pre; 34832_s_at Cluster Incl. AB018306:Homo sapiens mRNA for KIAA0763 protein, comple; 35827_at Cluster Incl. AB020712:Homo sapiens mRNA for KIAA0905 protein, complete; 36615_at Cluster Incl. M83751:Human arginine-rich protein (ARP) gene, complete c; 39128_r_at Cluster Incl. X73478:H. sapiens hPTPA mRNA /cds=(189,1160) /gb=X73478; 39473_r_at Cluster Incl. W29065:56g2 Homo sapiens cDNA /gb=W29065 /gi=1309094 /u; 39854_r_at Cluster Incl. AF055000:Homo sapiens clone 24519 unknown mRNA, partial; 39868_at Cluster Incl. AL046394:DKFZp434M217_r1 Homo sapiens cDNA, 5 end /clone; 40539_at Cluster Incl. U42391:Human myosin-IXb mRNA, complete cds /cds=(0,6068); 32525_r_at Cluster Incl. W29012:55a6 Homo sapiens cDNA /gb=W29012 /gi=1308969 /u; 1415_at D26561 /FEATURE=cds#3 /DEFINITION=D26561 Homo sapiens cellular DNA conta; 887_at M62302 /FEATURE= /DEFINITION=HUMGDF1 Human growth/differentiation factor; 689_at L02867 /FEATURE= /DEFINITION=HUMPPA Homo sapiens 62 kDa paraneoplastic an; 552_at U02570 /FEATURE= /DEFINITION=HSU02570 Human CDC42 GTPase-activating prote; 420_at X65633 /FEATURE=cds /DEFINITION=HSACTHR H. sapiens ACTH-R gene for adrenoc; 396_-fat X97671 /FEATURE=cds /DEFINITION=HSERYTHR H. sapiens mRNA for erythropoie; 259_s_at M16441 /FEATURE=cds#2 /DEFINITION=HUMTNFAB Human tumor necrosis factor; 179_at U38980 /FEATURE= /DEFINITION=U38980 Human PMS2 related (hPMSR6) mRNA, co; 121_at X69699 /FEATURE= /DEFINITION=HSPAX8A H. sapiens Pax8 mRNA.

Metagene 272; 37826_at Cluster Incl. AF020761:Homo sapiens stimulator of Fe transport mRNA, co; 38142_at Cluster Incl. U38904:Human zinc finger protein C2H2-25 mRNA, complete c; 38599_s_at Cluster Incl. AD001530:Homo sapiens XAP-5 mRNA, complete cds /cds=(75; 40756_at Cluster Incl. AF081280:Homo sapiens nucleoplasmin-3 (NPM3) mRNA, comple; 41632_at Cluster Incl. D38550:Human mRNA for KIAA0075 gene, partial cds /cds=(0,; 34768_at Cluster Incl. AL080080:Homo sapiens mRNA; cDNA DKFZp564E1962 (from clon; 40469_at Cluster Incl. AB011144:Homo sapiens mRNA for KIAA0572 protein, partial; 40473_at Cluster Incl. AF024636:Homo sapiens STE20-like kinase 3 (mst-3) mRNA, c; 40865_at Cluster Incl. U51166:Human G/T mismatch-specific thymine DNA glycosylas; 41755_at Cluster Incl. AB023194:Homo sapiens mRNA for KIAA0977 protein, complete; 32784_at Cluster Incl. AB011108:Homo sapiens mRNA for KIAA0536 protein, partial; 35296_at Cluster Incl. AB019036:Homo sapiens mRNA for geranylgeranyl pyrophospha; 35824_at Cluster Incl. AJ223321:Homo sapiens RP58 gene, complete CDS /cds=(523,2; 36190_at Cluster Incl. M63256:Human major Yo paraneoplastic antigen (CDR2) mRNA,; 36970_at Cluster Incl. D80004:Human mRNA for KIAA0182 gene, partial cds /cds=(0,; 37409_at Cluster Incl. U88666:Homo sapiens serine kinase SRPK2 mRNA, complete cd; 38767_at Cluster Incl. AF041037:Homo sapiens novel antagonist of FGF signaling (; 38824_at Cluster Incl. AF039103:Homo sapiens Tat-interacting protein TIP30 mRNA,; 41248_at Cluster Incl. AB014589:Homo sapiens mRNA for KIAA0689 protein, partial; 41601_at Cluster Incl. AA142964:zl43a07.s1 Homo sapiens cDNA, 3 end /clone=IMAG; 1987_at X76079 /FEATURE=exon#5 /DEFINITION=HSPDGF H. sapiens mRNA for platelet de; 1228_s_at U73682 /FEATURE= /DEFINITION=HSU73682 Human meningioma-expressed antig.

Metagene 273; 36422_s_at Cluster Incl. AF038198:Homo sapiens clone 23928 mRNA sequence /cds=UN; 37811_at Cluster Incl. AF042792:Homo sapiens alpha 2 delta calcium channel subun; 32033_at Cluster Incl. AL096780:Novel human gene mapping to chomosome 22p13.33 s; 35638_at Cluster Incl. D43638:Human mRNA for MTG8a protein, complete cds /cds=(4; 37908_at Cluster Incl. U31384:Human G protein gamma-11 subunit mRNA, complete cd; 35333_r_at Cluster Incl. AB024518:Homo sapiens mRNA for DVS27-related protein, c; 36681_at Cluster Incl. J02611:Human apolipoprotein D mRNA, complete cds /cds=(61; 1433_g_at U68019 /FEATURE= /DEFINITION=HSU68019 Homo sapiens mad protein homolog; 1336_s_at X06318 /FEATURE=cds /DEFINITION=HSPKCB1A Human mRNA for protein kinase.

Metagene 274; 35469_at Cluster Incl. X57830:H. sapiens serotonin 5-HT2 receptor mRNA /cds=(145,; 38911_at Cluster Incl. U41815:Human nucleoporin 98 (NUP98) mRNA, complete cds /c; 40308_at Cluster Incl. AI830496:wh51h03.x1 Homo sapiens cDNA, 3 end /clone=IMAG; 40655_at Cluster Incl. U94190:Homo sapiens Duo mRNA, complete cds /cds=(100,5091; 31884_at Cluster Incl. L40399:Homo sapiens (clone S240ii117 /zap112) mRNA, comple; 32060_at Cluster Incl. U15173:Homo sapiens BCL2 /adenovirus E1B 19 kD-interacting; 34278_at Cluster Incl. L18960:Human protein synthesis factor (eIF-4C) mRNA, comp; 35180_at Cluster Incl. AL050205:Homo sapiens mRNA; cDNA DKFZp586F1323 (from clon; 35213_at Cluster Incl. AF071185:Homo sapiens formin binding protein 21 mRNA, com; 35632_at Cluster Incl. U26710:Human cbl-b mRNA, complete cds /cds=(322,3270) /gb; 37615_at Cluster Incl. D86962:Human mRNA for KIAA0207 gene, complete cds /cds=(7; 37915_at Cluster Incl. AL080173:Homo sapiens mRNA; cDNA DKFZp434H071 (from clone; 38263_at Cluster Incl. X98330:H. sapiens mRNA for ryanodine receptor 2 /cds=(121,; 38277_at Cluster Incl. M29550:Human calcineurin A1 mRNA, complete cds /cds=(107,; 38978_at Cluster Incl. AF013758:Homo sapiens polyadenylate binding protein-inter; 39083_at Cluster Incl. U39318:Human E2 ubiquitin conjugating enzyme UbcH5C (UBCH; 39727_at Cluster Incl. AF023917:Homo sapiens protein tyrosine phosphatase PIR1 m; 39792_at Cluster Incl. AF000364:Homo sapiens heterogeneous nuclear ribonucleopro; 41133_at Cluster Incl. U32519:Human GAP SH3 binding protein mRNA, complete cds /; 32788_at Cluster Incl. D42063:Human mRNA for RanBP2 (Ran-binding protein 2), com; 33354_at Cluster Incl. AA630312:ac08f05.s1 Homo sapiens cDNA, 3 end /clone=IMAG; 35805_at Cluster Incl. AA447263:zw93f01.r1 Homo sapiens cDNA, 5 end /clone=IMAG; 36685_at Cluster Incl. W63793:zc55a10.r1 Homo sapiens cDNA, 5 end /clone=IMAGE-; 36942_at Cluster Incl. D79996:Human mRNA for KIAA0174 gene, complete cds /cds=(6; 37385_at Cluster Incl. U40763:Human Clk-associated RS cyclophilin CARS-Cyp mRNA,; 38073_at Cluster Incl. AB007858:Homo sapiens KIAA0398 mRNA, complete cds /cds=(1; 39507_at Cluster Incl. AL050366:Homo sapiens mRNA; cDNA DKFZp564A126 (from clone; 40236_at Cluster Incl. D29990:Human mRNA for cationic amino acid transporter 2,; 40931_at Cluster Incl. AL080084:Homo sapiens mRNA; cDNA DKFZp564G2362 (from clon; 33150_at Cluster Incl. AI126004:qc50e12.x1 Homo sapiens cDNA, 3 end /clone=IMAG; 1094_g_at M65254 /FEATURE= /DEFINITION=HUMP2B Protein phosphatase 2A 65 kDa regu; 831_at U28042 /FEATURE= /DEFINITION=HSU28042 Human DEAD box RNA helicase-like pr; 663_at L18960 /FEATURE= /DEFINITION=HUMEIF4C Human protein synthesis factor (eIF.

Metagene 275; 31522_f_at Cluster Incl. Z80779:H. sapiens H2B/g gene /cds=(0,380) /gb=Z80779 /gi; 31942_at Cluster Incl. AF045583:Homo sapiens tubby like protein 3 (TULP3) mRNA,; 1687_s_at X84213 /FEATURE=cds /DEFINITION=HSCEBP1 H. sapiens BAK mRNA for BC1-2 h.

Metagene 276; 36308_at Cluster Incl. D76435:Homo sapiens mRNA for Zic protein, complete cds /c; 40674_s_at Cluster Incl. S82986:HOXC6=homeodomain-containing protein {clone 211}; 39753_at Cluster Incl. X06256:Human mRNA for integrin alpha 5 subunit /cds=(23,3; 38404_at Cluster Incl. M55153:Human transglutaminase (TGase) mRNA, complete cds; 647_at L35545 /FEATURE=mRNA /DEFINITION=HUMECPC Homo sapiens endothelial cell pr.

Metagene 277; 34529_at Cluster Incl. W26760:12d6 Homo sapiens cDNA /gb=W26760 /gi=1305844 /ug=; 36281_at Cluster Incl. M96739:Human NSCL-1 mRNA sequence /cds=UNKNOWN /gb=M96739; 36743_at Cluster Incl. AL096739:Homo sapiens mRNA; cDNA DKFZp586H0623 (from clon; 37126_at Cluster Incl. M62800:Human 52-kD SS-A/Ro autoantigen mRNA, complete cds; 39239_at Cluster Incl. X13444:Human mRNA for CD8 beta-chain glycoprotein (CD8 be; 39318_at Cluster Incl. X82240:H. sapiens mRNA for Tcell leukemia/lymphoma 1 /cds=; 37935_at Cluster Incl. AF016369:Homo sapiens U4 /U6 small nuclear ribonucleoprote; 38067_at Cluster Incl. D86957:Human mRNA for KIAA0202 gene, partial cds /cds=(0,; 33170_at Cluster Incl. AB023179:Homo sapiens mRNA for KIAA0962 protein, partial.

Metagene 278; 35112_at Cluster Incl. AF071476:Homo sapiens regulator of G-protein signaling 9L; 36278_at Cluster Incl. X90846:H. sapiens mRNA for mixed lineage kinase 2 /cds=(28; 39930_at Cluster Incl. D83492:Homo sapiens mRNA for Eph-family protein, complete; 39967_at Cluster Incl. AB019527:Homo sapiens mRNA for LDOC1 protein, complete cd; 33756_at Cluster Incl. U39447:Human placenta copper monamine oxidase mRNA, compl; 34231_at Cluster Incl. AF074606:Homo sapiens histone acetyltransferase (HBO1) mR; 35678_at Cluster Incl. AB006757:Homo sapiens mRNA for PCDH7 (BH-Pcdh)c, complete; 37265_at Cluster Incl. D87074:Human mRNA for KIAA0237 gene, complete cds /cds=(4; 40776_at Cluster Incl. M63391:Human desmin gene, complete cds /cds=(80,1489) /gb; 34388_at Cluster Incl. Y11710:H. sapiens mRNA for extracellular matrix protein co; 36096_at Cluster Incl. AL080222:Homo sapiens mRNA; cDNA DKFZp566D1146 (from clon; 37765_at Cluster Incl. X54162:Human mRNA for a 64 Kd autoantigen expressed in th; 38474_at Cluster Incl. L00972:Human cystathionine-beta-synthase (CBS) mRNA /cds=; 38836_at Cluster Incl. AL008583:dJ327J16.2 (human ortholog of rat Neuronal Pentr; 39549_at Cluster Incl. AI743090:wg87a11.x1 Homo sapiens cDNA, 3 end /clone=IMAG; 32526_at Cluster Incl. AA149644:zl39d08.s1 Homo sapiens cDNA, 3 end /clone=IMAG; 32582_at Cluster Incl. AF001548:Human Chromosome 16 BAC clone CIT987SK-A-815A9 /; 33123_at Cluster Incl. L10379:Human (clone CTG-B45d) mRNA sequence /cds=UNKNOWN; 1361_at U40705 /FEATURE= /DEFINITION=HSU40705 Homo sapiens telomeric repeat bind.

Metagene 279; 40180_at Cluster Incl. AB000732:Homo sapiens gene for insulin receptor substrate; 538_at S53911 /FEATURE= /DEFINITION=S53911 CD34=glycoprotein expressed in lympho; 381_s_at Y10055 /FEATURE=cds /DEFINITION=HSP110DEL H. sapiens mRNA for phosphoino.

Metagene 280; 34505_at Cluster Incl. AL109725:Homo sapiens mRNA full length insert cDNA clone; 32094_at Cluster Incl. AB017915:Homo sapiens mRNA for condoroitin 6-sulfotransfe; 35615_at Cluster Incl. D50914:Human mRNA for KIAA0124 gene, partial cds /cds=(0,; 35648_at Cluster Incl. AB007902:Homo sapiens KIAA0442 mRNA, partial cds /cds=(0,; 38977_at Cluster Incl. U89436:Human tyrosyl-tRNA synthetase mRNA, complete cds /; 39796_at Cluster Incl. U11292:Human Ki nuclear autoantigen mRNA, complete cds /c; 32848_at Cluster Incl. AF020736:Homo sapiens ATPase homolog mRNA, complete cds /; 34883_at Cluster Incl. D87451:Human mRNA for KIAA0262 gene, complete cds /cds=(6; 35844_at Cluster Incl. D79206:Homo sapiens gene for ryudocan core protein, exon1; 38822_at Cluster Incl. AB011420:Homo sapiens mRNA for DRAK1, complete cds /cds=(; 39824_at Cluster Incl. AI391564:tg16b02.x1 Homo sapiens cDNA, 3 end /clone=IMAG; 758_at D38128 /FEATURE=expanded cds /DEFINITION=HUMIP2 Homo sapiens IP gene for.

Metagene 281; 34990_at Cluster Incl. AB022660:Homo sapiens mRNA for SET-binding protein (SEB),; 38951_at Cluster Incl. M59217:Human collagen type XIII alpha-1 mRNA, complete cd; 38952_s_at Cluster Incl. M33653:Human (clones HT-[125,133]) alpha-2 type IV coll; 39665_at Cluster Incl. U33267:Human glycine receptor beta subunit (GLRB) mRNA, c; 40330_at Cluster Incl. AL050031:Homo sapiens mRNA; cDNA DKFZp566E0224 (from clon; 40746_at Cluster Incl. L20814:Human glutamate receptor 2 (HBGR2) mRNA, complete; 32056_at Cluster Incl. AF035811:Homo sapiens protein H5 (H5) mRNA, complete cds; 32689_s_at Cluster Incl. D86096:Human DNA for prostaglandin E receptor EP3 subty; 36044_at Cluster Incl. AF022912:Homo sapiens cGMP phosphodiesterase delta subuni; 36925_at Cluster Incl. L26336:Human heat shock protein HSPA2 gene, complete cds; 40455_at Cluster Incl. AB020637:Homo sapiens mRNA for KIAA0830 protein, partial; 40764_at Cluster Incl. M22632:Human mitochondrial aspartate aminotransferase mRN; 36990_at Cluster Incl. X04741:Human mRNA for protein gene product (PGP) 9.5 /cds; 38743_f_at Cluster Incl. X06409:Human mRNA fragment for activated c-raf-1 (exons; 39160_at Cluster Incl. D90086:Human pyruvate dehydrogenase (EC 1.2.4.1) beta sub; 1969_s_at X77743 /FEATURE=cds /DEFINITION=HSCDKAK H. sapiens CDK activating kinas; 1586_at M35878 /FEATURE=expanded-cds /DEFINITION=HUMIBP3 Human insulin-like grow; 645_at L26336 /FEATURE=cds /DEFINITION=HUMHSPA2A Human heat shock protein HSPA2.

Metagene 282; 36314_at Cluster Incl. U04806:Human FLT3 /FLK2 ligand mRNA, complete cds /cds=(92; 40646_at Cluster Incl. U20350:Human G protein-coupled receptor V28 mRNA, complet; 41400_at Cluster Incl. K02581:Human thymidine kinase mRNA, complete cds /cds=(57; 41475_at Cluster Incl. U91512:Human adhesion molecule ninjurin mRNA, complete cd; 37268_at Cluster Incl. U43368:Human VEGF related factor isoform VRF186 precursor; 37600_at Cluster Incl. U68186:Human extracellular matrix protein 1 mRNA, complet; 37963_at Cluster Incl. X52151:Homo sapiens arylsulphatase A mRNA, complete cds /; 39014_at Cluster Incl. D84239:Homo sapiens mRNA for IgG Fc binding protein, comp; 39326_at Cluster Incl. Z71460:H. sapiens mRNA for vacuolar-type H(+)-ATPase 115 k; 41725_at Cluster Incl. U89896:Homo sapiens casein kinase I gamma 2 mRNA, complet; 34412_s_at Cluster Incl. U59632:Homo sapiens H5 mRNA, partial cds; and platelet; 36993_at Cluster Incl. M33210:Human colony stimulating factor 1 receptor (CSF1R); 37360_at Cluster Incl. U66711:Human Ly-6-related protein (9804) gene, complete c; 39541_at Cluster Incl. W52003:zc92e10.r1 Homo sapiens cDNA, 5 end /clone=IMAGE-; 40906_at Cluster Incl. AC002310:Human Chromosome 16 BAC clone CIT987SK-A-635H112; 1771_s_at J03278 /FEATURE= /DEFINITION=HUMPDGFRA Human platelet-derived growth f; 1505_at D00596 /FEATURE=cds /DEFINITION=HUMTS1 Homo sapiens gene for thymidylate; 1242_at U15655 /FEATURE= /DEFINITION=HSU15655 Human ets domain protein ERF mRNA,; 1087_at M60459 /FEATURE= /DEFINITION=HUMERYTH Human erythropoietin receptor mRNA.

Metagene 283; 31323_r_at Cluster Incl. S83374:glutamate transporter II variant B/HBGT IIB {5 r; 31531_g_at Cluster Incl. U89344:Human acetyl-CoA carboxylase (ACC2) mRNA, comple; 31564_at Cluster Incl. S65761:anti-colorectal carcinoma heavy chain=glycoprotein; 31775_at Cluster Incl. X65018:H. sapiens mRNA for lung surfactant protein D /cds=; 33027_at Cluster Incl. W27906:39d11 Homo sapiens cDNA /gb=W27906 /gi=1307854 /ug; 32353_at Cluster Incl. M13143:Nucleotide sequence of the cDNA insert of lambda P; 35004_at Cluster Incl. U31875:Human Hep27 protein mRNA, complete cds /cds=(433,1; 37486_f_at Cluster Incl. U68385:Human Meis1-related protein 2 (MRG2), mRNA, part; 37771_at Cluster Incl. AB020688:Homo sapiens mRNA for KIAA0881 protein, complete; 38203_at Cluster Incl. U69883:Human calcium-activated potassium channel hSK1 (SK; 40739_at Cluster Incl. M83670:Human carbonic anhydrase IV mRNA, complete cds /cd; 41614_at Cluster Incl. AB014608:Homo sapiens mRNA for KIAA0708 protein, partial; 31835_at Cluster Incl. M13149:Human histidine-rich glycoprotein mRNA, complete c; 32054_at Cluster Incl. AF048732:Homo sapiens cyclin T2b mRNA, complete cds /cds=; 36530_g_at Cluster Incl. AI885381:wl93b01.x1 Homo sapiens cDNA, 3 end /clone=IM; 40769_r_at Cluster Incl. D14689:Human mRNA for KIAA0023 gene, complete cds /cds=; 33869_at Cluster Incl. AL080218:Homo sapiens mRNA; cDNA DKFZp586N1323 (from clon; 36148_at Cluster Incl. U48437:Human amyloid precursor-like protein 1 mRNA, compl; 39877_at Cluster Incl. AI832082:td12c04.x1 Homo sapiens cDNA, 3 end /clone=IMAG; 41491_s_at Cluster Incl. AB028944:Homo sapiens mRNA for KIAA1021 protein, partia; 898_s_at L37360 /FEATURE= /DEFINITION=HUMEFL2 Homo sapiens (clone hEHK1-L) EHK1; 740_at D49394 /FEATURE= /DEFINITION=HUMS5HT3RA Human mRNA for serotonin 5-HT3 re.

Metagene 284; 31440_at Cluster Incl. Z47363:H. sapiens TCF-1 mRNA for T cell factor 1 splice fo; 33685_at Cluster Incl. AC004755:Homo sapiens chromosome 19, fosmid 37502 /cds=(0; 32918_at Cluster Incl. AL080182:Homo sapiens mRNA; cDNA DKFZp434O151 (from clone; 37332_r_at Cluster Incl. U24267:Human pyrroline-5-carboxylate dehydrogenase (P5C; 38461_at Cluster Incl. X83957:H. sapiens mRNA for nebulin /cds=(440,20449) /gb=X8; 39511_at Cluster Incl. AB011399:Homo sapiens gene for AF-6, complete cds /cds=(0.

Metagene 285; 36745_at Cluster Incl. AF035308:Homo sapiens clone 23798 and 23825 mRNA sequence; 1007_-sat U48705 /FEATURE=mRNA /DEFINITION=HSU48705 Human receptor tyrosine kina.

Metagene 286; 39283_at Cluster Incl. X83543:H. sapiens APXL mRNA /cds=(90,4940) /gb=X83543 /gi=; 37213_at Cluster Incl. X90392:H. sapiens mRNA for DNase X gene /cds=(794,1702) /g; 435_g_at X03473 /FEATURE=cds /DEFINITION=HSHIS10G Human gene for histone H1(0).

Metagene 287; 31344_at Cluster Incl. AF063725:Homo sapiens clone BCSynL38 immunoglobulin lambd; 38683_s_at Cluster Incl. AB029008:Homo sapiens mRNA for KIAA1085 protein, partia; 40481_r_at Cluster Incl. M14333:Homo sapiens c-syn protooncogene mRNA, complete; 37721_at Cluster Incl. U79262:Human deoxyhypusine synthase mRNA, complete cds /c; 39837_s_at Cluster Incl. AC004877:Homo sapiens PAC clone DJ0751H13 from 7q35-qte; 41314_at Cluster Incl. AF047042:Homo sapiens citrate synthase mRNA, complete cds; 33182_at Cluster Incl. AI018523:ou47d07.x1 Homo sapiens cDNA, 3 end /clone=IMAG; 1397_at L32976 /FEATURE= /DEFINITION=HUMMLK3A Human protein kinase (MLK-3) mRNA,; 1117_at L27943 /FEATURE=mRNA /DEFINITION=HUMCYDE Homo sapiens cytidine deaminase; 560_s_at M63589 /FEATURE=mRNA#5 /DEFINITION=HUMSCL7 Human stem cell leukemia gen.

Metagene 288; 32452_at Cluster Incl. X66357:H. sapiens mRNA cdk3 for serine/threonine protein k; 34558_at Cluster Incl. U30185:Human orphan opioid receptor mRNA, complete cds /c; 35091_at Cluster Incl. AA706226:ah28a07.s1 Homo sapiens cDNA, 3 end /clone=1240; 35597_at Cluster Incl. AJ000480:Homo sapiens mRNA for C8FW phosphoprotein /cds=(; 36361_at Cluster Incl. U34051:Human cyclin-dependent kinase 5 activator isoform; 32909_at Cluster Incl. U46569:Human aquaporin-5 (AQP5) gene /cds=(0,797) /gb=U46; 37088_at Cluster Incl. AF059681:Homo sapiens serine/threonine kinase 13 (STK13); 37129_at Cluster Incl. AI936758:wp69b11.x1 Homo sapiens cDNA, 3 end /clone=IMAG; 38254_at Cluster Incl. AB020689:Homo sapiens mRNA for KIAA0882 protein, partial; 38716_at Cluster Incl. AB018330:Homo sapiens mRNA for KIAA0787 protein, partial; 41190_at Cluster Incl. U83598:Human death domain receptor 3 soluble form (DDR3); 38058_at Cluster Incl. Z22865:H. sapiens dermatopontin mRNA, complete CDS /cds=(1; 40622_r_at Cluster Incl. AL096740:Homo sapiens mRNA; cDNA DKFZp586K2123 (from cl; 40919_at Cluster Incl. M81830:Human somatostatin receptor isoform 2 (SSTR2) gene; 40935_at Cluster Incl. W28516:47h7 Homo sapiens cDNA /gb-W28516 /gi=1308464 /ug=; 41817_g_at Cluster Incl. AL049851:Human DNA sequence from clone 889J22B on chrom; 1118_at L28175 /FEATURE= /DEFINITION=HUMPERE Homo sapiens prostaglandin E2 recep; 908_at M14660 /FEATURE=cds /DEFINITION=HUMISG2 Human ISG-54K gene (interferon st; 598_at M60299 /FEATURE=cds /DEFINITION=HUMCOLII Human alpha-1 collagen type II g; 160038_s_at J00268 /FEATURE=gene /DEFINITION=HUMINS02 Human insulin gene: repeti.

Metagene 289; 37127_at Cluster Incl. AB023143:Homo sapiens mRNA for KIAA0926 protein, complete; 37511_at Cluster Incl. AB030506:Homo sapiens mRNA for B9, complete cds /cds=(158; 41375_at Cluster Incl. AJ245416:Homo sapiens mRNA for G7b protein (G7b gene, loc; 35161_at Cluster Incl. AB020667:Homo sapiens mRNA for KIAA0860 protein, complete; 35206_at Cluster Incl. AF049105:Homo sapiens centrosomal Nek2-associated protein; 36009_at Cluster Incl. AF091092:Homo sapiens clone 683 unknown mRNA, complete se; 36500_at Cluster Incl. AF027974:Homo sapiens clone LM1955 H105e3 gene, partial c; 36812_at Cluster Incl. U92715:Homo sapiens breast cancer antiestrogen resistance; 40225_at Cluster Incl. D88435:Homo sapiens mRNA for HsGAK, complete cds /cds=(0,; 40244_s_at Cluster Incl. AI743654:wg41d01.x1 Homo sapiens cDNA, 3 end /clone=IM; 40997_at Cluster Incl. AI660963:wf20e04.x1 Homo sapiens cDNA, 3 end /clone=IMAG.

Metagene 290; 31446_s_at Cluster Incl. D89501:Human PBI gene, complete cds /cds=(14,418) /gb=D; 32956_at Cluster Incl. AC002550:Human Chromosome 16 BAC clone CIT987SK-A-101F10; 38529_at Cluster Incl. X68968:H. sapiens mRNA for acetyl-CoA carboxylase /cds=(0,; 33258_g_at Cluster Incl. D26535:Human gene for dihydrolipoamide succinyltransfer; 34200_at Cluster Incl. X83378:H. sapiens mRNA for putative chloride channel /cds=; 37262_at Cluster Incl. AF061324:untitled /cds=(0,4649) /gb=AF061324 /gi=312717⁴ ; 39715_at Cluster Incl. W28214:45f7 Homo sapiens cDNA /gb=W28214 /gi=1308297 /ug=; 33852_at Cluster Incl. M77142:Human polyadenylate binding protein (TIA-1) mRNA,; 34364_at Cluster Incl. AF042385:Homo sapiens cyclophilin-33A (CYP-33) mRNA, comp; 39101_at Cluster Incl. S73840:Homo sapiens type IIA myosin heavy chain mRNA, par; 40242_at Cluster Incl. L36529:Human (clone N5-4) protein p84 mRNA, complete cds; 1533_at U63139 /FEATURE= /DEFINITION=HSU63139 Homo sapiens Rad50 (Rad50) mRNA, c; 871_s_at M95585 /FEATURE=mRNA /DEFINITION=HUMHLF Human hepatic leukemia factor (; 639_s_at L47345 /FEATURE= /DEFINITION=HUMELONA Homo sapiens elongin A mRNA, comp; 394_at X92106 /FEATURE=cds /DEFINITION=HSBLEO H. sapiens mRNA for bleomycin hydro; 108_at Z95624 /FEATURE=cds /DEFINITION=HSU237H1 Human DNA sequence from cosmid.

Metagene 291; 33943_at Cluster Incl. L20941:Human ferritin heavy chain mRNA, complete cds /cds; 34530_at Cluster Incl. W73822:zd52f10.s1 Homo sapiens cDNA, 3 end /clone=IMAGE-; 37147_at Cluster Incl. AF020044:Homo sapiens lymphocyte secreted C-type lectin p; 38237_at Cluster Incl. M64099:Human gamma-glutmyl transpeptidase-related protein; 33308_at Cluster Incl. M15182:Human beta-glucuronidase mRNA, complete cds /cds=(; 34670_at Cluster Incl. U60899:Human lysosomal alpha-mannosidase (manE) gene /cds; 34689_at Cluster Incl. AJ243797:Homo sapiens mRNA for deoxyribonuclease III (drn; 36529_at Cluster Incl. AI885381:wl93b01.x1 Homo sapiens cDNA, 3 end /clone=IMAG; 36902_at Cluster Incl. X61587:H. sapiens rhoG mRNA for GTPase /cds=(129,704) /gb=; 37181_at Cluster Incl. X76538:H. sapiens Mpv17 mRNA /cds=(29,559) /gb=X76538 /gi=; 38003_s_at Cluster Incl. U94905:Human diacylglycerol kinase zeta mRNA, alternati; 39007_at Cluster Incl. M55593:Human collagenase type IV (CLG4) gene /cds=(289,22; 39062_at Cluster Incl. AL008726:dJ337O18.2 (Lysosomal Protective Protein precurs; 39340_at Cluster Incl. M16424:Human beta-hexosaminidase alpha chain (HEXA) gene; 39798_at Cluster Incl. R87876:yo45h01.r1 Homo sapiens cDNA, 5 end /clone=IMAGE-; 41140_at Cluster Incl. U05875:Human clone pSK1 interferon gamma receptor accesso; 32203_at Cluster Incl. AA160708:zo72c02.r1 Homo sapiens cDNA, 5 end /clone=IMAG; 34361_at Cluster Incl. U33849:Human lymphoma proprotein convertase (LPC) mRNA, c; 35770_at Cluster Incl. D16469:Human mRNA for ORF, Xq terminal portion /cds=(1353; 37754_at Cluster Incl. L13210:Human Mac-2 binding protein mRNA, complete cds /cd; 38487_at Cluster Incl. D87433:Human mRNA for KIAA0246 gene, partial cds /cds=(0,; 39170_at Cluster Incl. AL049957:Homo sapiens mRNA; cDNA DKFZp564J0323 (from clon; 39918_at Cluster Incl. AF042379:Homo sapiens spindle pole body protein spc97 hom; 40274_at Cluster Incl. U48213:Human D-site binding protein gene, promoter region; 1976_s_at X06292 /FEATURE=mRNA /DEFINITION=HSFESFPS Human c-fes/fps proto-oncoge; 1398_g_at L32976 /FEATURE= /DEFINITION=HUMMLK3A Human protein kinase (MLK-3) mRN; 906_at L78440 /FEATURE=mRNA /DEFINITION=HUMSTAT4R Homo sapiens STAT4 mRNA, compl; 816_g_at U70987 /FEATURE= /DEFINITION=HSU70987 Human GAP binding protein p62dok; 464_s_at U72882 /FEATURE= /DEFINITION=HSU72882 Human interferon-induced leucine; 384_at X71874 /FEATURE=cds#1 /DEFINITION=HSPROSCHY H. sapiens genes for proteasom.

Metagene 292; 36835_at Cluster Incl. U33052:Human lipid-activated, protein kinase PRK2 mRNA, c; 41192_at Cluster Incl. AF091090:Homo sapiens clone 669 unknown mRNA, complete se; 37680_at Cluster Incl. U81607:Homo sapiens gravin mRNA, complete cds /cds=(191,5.

Metagene 293; 34466_at Cluster Incl. Y12505:H. sapiens mRNA for serotonin receptor 5-HT4B, spli; 1717_s_at U45878 /FEATURE= /DEFINITION=HSU45878 Human inhibitor of apoptosis pro.

Metagene 294; 32728_at Cluster Incl. X81438:H. sapiens mRNA for amphiphysin /cds=(74,2161) /gb=; 33244_at Cluster Incl. U07223:Human beta2-chimaerin mRNA, complete cds /cds=(444; 37283_at Cluster Incl. X82209:H. sapiens MN1 mRNA /cds=(887,4915) /gb=X82209 /gi=; 39026_r_at Cluster Incl. AF052114:Homo sapiens clone 23887 mRNA sequence /cds=UN; 39350_at Cluster Incl. U50410:Human heparan sulphate proteoglycan (OCI5) mRNA, c; 34820_at Cluster Incl. M57399:Human nerve growth factor (HBNF-1) mRNA, complete; 35324_at Cluster Incl. AB011538:Homo sapiens mRNA for MEGF5, partial cds /cds=(0; 35354_at Cluster Incl. AL022326:dJ333H23.2.2 (Synaptogyrin 1A (SYNGR1A)) /cds=(4; 37669_s_at Cluster Incl. U16799:Human Na,K-ATPase beta-1 subunit mRNA, complete; 38086_at Cluster Incl. AB007935:Homo sapiens mRNA for KIAA0466 protein, partial; 40199_at Cluster Incl. M97676:Homo sapiens (region 7) homeobox protein (HOX7) mR; 41245_at Cluster Incl. D49493:Human gene for human bone morphogenetic protein-3b; 32612_at Cluster Incl. X04412:Human mRNA for plasma gelsolin /cds=(14,2362) /gb=; 1290_g_at L02321 /FEATURE= /DEFINITION=HUMGSTM5 Human glutathione S-transferase; 234_s_at M57399 /FEATURE= /DEFINITION=HUMHBNF1 Human nerve growth factor (HBNF-1; 214_at M97676 /FEATURE= /DEFINITION=HUMHOX7 Homo sapiens (region 7) homeobox pro.

Metagene 295; 41866_s_at Cluster Incl. AF022728:Homo sapiens beta-dystrobrevin (BDTN) mRNA, co; 33382_at Cluster Incl. M92449:Human LTR mRNA, 3 end of coding region and 3 fla; 41583_at Cluster Incl. AC004770:Homo sapiens chromosome 11, BAC CIT-HSP-311e8 (B; 796_i_at X66358 /FEATURE=cds#2 /DEFINITION=HSSTHPKB H. sapiens mRNA KKIALRE for s.

Metagene 296; 31477_at Cluster Incl. L08044:Human intestinal trefoil factor mRNA, complete cds; 35934_at Cluster Incl. L19161:Human translation initiation factor eIF-2 gamma su; 36732_at Cluster Incl. AI004207:ot94g05.x1 Homo sapiens cDNA, 3 end /clone=IMAG; 37139_at Cluster Incl. AF040628:Homo sapiens ectodysplasin-A (EDA) mRNA, complet; 37472_at Cluster Incl. U60337:Homo sapiens beta-mannosidase mRNA, complete cds /; 39271_at Cluster Incl. AA461365:zx70e07.r1 Homo sapiens cDNA, 5 end /clone=IMAG; 40512_at Cluster Incl. X51408:Human mRNA for n-chimaerin /cds=(552,1451) /gb=X51; 40835_at Cluster Incl. AB012922:Homo sapiens MTA1-L1 gene, complete cds /cds=(72; 37686_s_at Cluster Incl. Y09008:H. sapiens mRNA for uracil-DNA glycosylase /cds=(; 1272_at L19161 /FEATURE= /DEFINITION=HUMIEF2G Human translation initiation facto; 577_at M94250 /FEATURE=expanded_cds /DEFINITION=HUMMKXX Human retinoic acid indu.

Metagene 297; 31471_at Cluster Incl. AL031393:Human DNA sequence from clone 733D15 on chromoso; 31567_at Cluster Incl. S82769:GABAA receptor gamma 3 subunit [human, fetal brain; 38582_at Cluster Incl. AI961220:wt15b04.x1 Homo sapiens cDNA, 3 end /clone=IMAG; 41404_at Cluster Incl. AJ010119:Homo sapiens mRNA for Ribosomal protein kinase B; 32622_at Cluster Incl. L36983:Homo sapiens dynamin (DNM) mRNA, complete cds /cds; 33750_at Cluster Incl. X97198:H. sapiens mRNA for receptor phosphate PCP-2 /cds=(; 34769_at Cluster Incl. U82535:Human fatty acid amide hydrolase mRNA, complete cd; 35243_at Cluster Incl. AA535884:nf90d04.s1 Homo sapiens cDNA, 3 end /clone=IMAG; 38703_at Cluster Incl. AF005050:Homo sapiens aspartyl aminopeptidase mRNA, compl; 39700_at Cluster Incl. AI961929:wt39g02.x1 Homo sapiens cDNA, 3 end /clone=IMAG; 40821_at Cluster Incl. M61832:Human S-adenosylhomocysteine hydrolase (AHCY) mRNA; 33438_at Cluster Incl. AL049981:Homo sapiens mRNA; cDNA DKFZp564D012 (from clone; 36179_at Cluster Incl. U12779:Human MAP kinase activated protein kinase 2 mRNA,; 37753_at Cluster Incl. U66617:Human SWI/SNF complex 60 KDa subunit (BAF60a) mRNA; 40596_at Cluster Incl. U76366:Human Treacher Collins syndrome (TCOF1) mRNA, comp; 40925_at Cluster Incl. AA554945:ni36d11.s1 Homo sapiens cDNA, 3 end /clone=IMAG; 1564_at M63167 /FEATURE= /DEFINITION=HUMRACPC Human rac protein kinase alpha mRN; 1225_gnat X66363 /FEATURE=cds /DEFINITION=HSSTHPKD H. sapiens mRNA PCTAIRE-1 for; 247_s_at M26856 /FEATURE=cds /DEFINITION=HUMCP210H Human 21-hydroxylase B gene,.

Metagene 298; 32406_at Cluster Incl. AB020696:Homo sapiens mRNA for KIAA0889 protein, complete; 37476_at Cluster Incl. AA650210:ns88b12.s1 Homo sapiens cDNA /clone=IMAGE-119068; 38175_at Cluster Incl. AB014567:Homo sapiens mRNA for KIAA0667 protein, partial; 39925_at Cluster Incl. M95610:Human alpha 2 type IX collagen (COL9A2) mRNA, part; 40301_at Cluster Incl. AI703188:wd92g12.x1 Homo sapiens cDNA, 3 end /clone=IMAG; 41055_at Cluster Incl. AB002361:Human mRNA for KIAA0363 gene, partial cds /cds=(; 31875_at Cluster Incl. AF055024:Homo sapiens clone 24763 mRNA sequence /cds=UNKN; 38625_g_at Cluster Incl. AF054506:Homo sapiens erythroid K—Cl cotransporter spli; 40848_g_at Cluster Incl. AB018293:Homo sapiens mRNA for KIAA0750 protein, comple; 41235_at Cluster Incl. AL022312:dJ1104E15.2 (activating transcription factor 4 (; 33430_at Cluster Incl. AA143321:zo37d01.s1 Homo sapiens cDNA, 3 end /clone=IMAG; 36120_at Cluster Incl. X63657:H. sapiens fvt1 mRNA /cds=(107,1105) /gb=X63657 /gi; 38433_at Cluster Incl. M76125:Human tyrosine kinase receptor (axl) mRNA, complet; 39175_at Cluster Incl. D25328:Human mRNA for platelet-type phosphofructokinase,; 40231_at Cluster Incl. AI193899:qe80f08.x1 Homo sapiens cDNA, 3 end /clone=IMAG; 1881_at Oncogene Aml1-Evi-1, Fusion Activated; 1363_at M87770 /FEATURE= /DEFINITION=HUMKSAMI Human fibroblast growth factor rec.

Metagene 299; 34914_at Cluster Incl. U94778:Human PEST phosphatase interacting protein homolog; 34193_at Cluster Incl. AF002246:Homo sapiens neural cell adhesion molecule (CALL; 32819_at Cluster Incl. AJ223352:Homo sapiens mRNA for for histone H2B, clone pjG; 33387_at Cluster Incl. AB007854:Homo sapiens KIAA0394 mRNA, complete cds /cds=(1; 37018_at Cluster Incl. AI189287:qd05c04.x1 Homo sapiens cDNA, 3 end /clone=IMAG; 1186_at D49493 /FEATURE=_expanded cds /DEFINITION=D49493 Human gene for human bon; 820_at U77604 /FEATURE= /DEFINITION=HSU77604 Homo sapiens microsomal glutathione.

Metagene 300; 33029_at Cluster Incl. AF038461:Homo sapiens 12R-lipoxygenase mRNA, complete cds; 40815_g_at Cluster Incl. L40586:Homo sapiens iduronate-2-sulphatase (IDS) mRNA,; 37022_at Cluster Incl. U41344:Human prolargin (PRELP) gene, 5 flanking sequence; 1663_at Retinoic Acid Receptor, Gamma 2. Metagene 301; 31589_at Cluster Incl. AL031983:dJ271M21.2 (hs6M1-12 (7 transmembrane receptor (; 32397_r_at Cluster Incl. AA079018:zm94e12.s1 Homo sapiens cDNA, 3 end /clone=IM; 35068_at Cluster Incl. U88063:Human Agouti related protein (Art) mRNA, complete; 35104_r_at Cluster Incl. M26682:Human T-cell translocation gene 1 (Ttg-1) mRNA,; 36429_at Cluster Incl. Y13583:Homo sapiens mRNA for G-protein coupled receptor /; 33469_r_at Cluster Incl. X68679:H. sapiens mRNA for DOWN 16 /cds=(23,1018) /gb=X; 37165_f_at Cluster Incl. X54534:Human mRNA for erythrocyte membrane protein Rh30; 38882_r_at Cluster Incl. AF096870:Homo sapiens estrogen-responsive B box protein; 41034_s_at Cluster Incl. U92315:Homo sapiens hydroxysteroid sulfotransferase SUL; 35691_r_at Cluster Incl. U18760:Human nuclear factor I (NFI) mRNA, clone AT2, pa; 37629_at Cluster Incl. M55268:Human casein kinase II alpha subunit mRNA, complet; 39689_at Cluster Incl. AI362017:qy39a10.x1 Homo sapiens cDNA, 3 end /clone=IMAG; 40412_at Cluster Incl. AA203476:zx55e01.r1 Homo sapiens cDNA, 5 end /clone=IMAG; 41125_r_at Cluster Incl. D45421:Human mRNA for phosphodiesterase 1 alpha, comple; 36094_at Cluster Incl. M21984:Human (clone PWHTnT16) skeletal muscle Troponin T; 36123_at Cluster Incl. D87292:Homo sapiens mRNA for rhodanese, complete cds /cds; 37335_at Cluster Incl. U41668:Human deoxyguanosine kinase mRNA, complete cds /cd; 39198_s_at Cluster Incl. W27763:37c8 Homo sapiens cDNA /gb=W27763 /gi=1307711 /u; 39815_at Cluster Incl. AA883101:am24d05.s1 Homo sapiens cDNA, 3 end /clone=IMAG; 40883_at Cluster Incl. W28230:43h12 Homo sapiens cDNA /gb=W28230 /gi=1308178 /ug; 41305_at Cluster Incl. M95549:Homo sapiens sodium/glucose cotransporter-like pro; 41546_at Cluster Incl. AI738463:wi32b08.x1 Homo sapiens cDNA, 3 end /clone=IMAG; 32538_at Cluster Incl. S95936:transferrin [human, liver, mRNA, 2347 nt]/cds=(79; 32594_at Cluster Incl. AF026291:Homo sapiens chaperonin containing t-complex pol; 2030_at N95031 /FEATURE= /DEFINITION=N95031 zb32b01.s1 Soares_parathyroid-tumor_{—}; 1742_at S61953 /FEATURE= /DEFINITION=S61953 c-erbB3=receptor tyrosine kinase {a1; 1707_g_at U01337 /FEATURE=expanded_cds /DEFINITION=HSU01337 Human Ser/Thr protei; 1569_r_at L42243 /FEATURE=exon#3 /DEFINITION=HUMIFNAM08 Homo sapiens (clone 51H8; 778_s_at D16827 /FEATURE=cds /DEFINITION=HUMSSTR5 Human gene for fifth somatosta; 441_s_at X13967 /FEATURE=cds /DEFINITION=HSLIF Human mRNA for leukaemia inhibito; 403_s_at X69878 /FEATURE=cds /DEFINITION=HSFLT4X H. sapiens Flt4 mRNA for transme; 225_at M31328 /FEATURE=mRNA /DEFINITION=HUMGNBPB3 Human guanine nucleotide-bindi.

Metagene 302; 31608_g_at Cluster Incl. AJ002428:Homo sapiens VDAC1 pseudogene /cds=(0,853) /gb; 31682_s_at Cluster Incl. D32039:Human pgH3 mRNA for proteoglycan PG-M(V3), compl; 31951_s_at Cluster Incl. Z48501:H. sapiens mRNA for polyadenylate binding protein; 34648_at Cluster Incl. Z12830:H. sapiens mRNA for SSR alpha subunit /cds=(29,889); 35099_at Cluster Incl. AF019225:Homo sapiens apolipoprotein L mRNA, complete cds; 36383_at Cluster Incl. M17254:Human erg2 gene encoding erg2 protein, complete cd; 32892_at Cluster Incl. X85106:H. sapiens mRNA for ribosomal S6 kinase /cds=(174,2; 34017_s_at Cluster Incl. AF022853:untitled /cds=(0,4370) /gb=AF022853 /gi=258577; 35967_at Cluster Incl. M69238:Human aryl hydrocarbon receptor nuclear translocat; 38580_at Cluster Incl. U43083:Human G alpha-q (Gaq) mRNA, complete cds /cds=(220; 41390_at Cluster Incl. X69086:H. sapiens mRNA for utrophin /cds=(0,10301) /gb=X69; 37531_at Cluster Incl. D86965:Human mRNA for KIAA0210 gene, complete cds /cds=(1; 40096_at Cluster Incl. D14710:Human mRNA for ATP synthase alpha subunit, complet; 40440_at Cluster Incl. AL080119:Homo sapiens mRNA; cDNA DKFZp564M2423 (from clon; 41144_g_at Cluster Incl. U12022:Human calmodulin (CALM1) gene /cds=(199,648) /gb; 33372_at Cluster Incl. AI89226:qd04h11.x1 Homo sapiens cDNA, 3 end /clone=IMAG; 34339_at Cluster Incl. AB009282:Homo sapiens mRNA for cytochrome b5, partial cds; 34855_at Cluster Incl. X76770:H. sapiens PAP mRNA /cds=UNKNOWN /gb=X76770 /gi=556; 35784_at Cluster Incl. U64520:Human synaptobrevin-3 mRNA, complete cds /cds=(24,; 40618_at Cluster Incl. H15872:ym22b12.r1 Homo sapiens cDNA, 5 end /clone=IMAGE-; 2012_s_at U34994 /FEATURE= /DEFINITION=HSU34994 Homo sapiens DNA dependent prote; 1920_s_at X77794 /FEATURE=cds /DEFINITION=HSCYCG1 H. sapiens mRNA for cyclin G1; 1839_at Ras-Like Protein Tc4; 1844_s_at L05624 /FEATURE= /DEFINITION=HUMMKK Homo sapiens MAP kinase kinase mRN; 1824_s_at J05614 /FEATURE=mRNA /DEFINITION=HUMPCNAPRM Human proliferating cell n; 622_at M28212 /FEATURE= /DEFINITION=HUMRAB6A Homo sapiens GTP-binding protein (R; 504_at U39318 /FEATURE= /DEFINITION=HSU39318 Human E2 ubiquitin conjugating enzy; 475_at U50062 /FEATURE= /DEFINITION=HSU50062 Homo sapiens RIP protein kinase mRN; 466_at U77948 /FEATURE= /DEFINITION=HSU77948 Human Bruton s tyrosine kinase-asso; 398_at X98743 /FEATURE=cds /DEFINITION=HSRNAHELC H. sapiens mRNA for RNA helicase; 353_at D30037 /FEATURE= /DEFINITION=HUMPITPB Human mRNA for phosphatidylinositol; 263_g_at M21154 /FEATURE=mRNA /DEFINITION=HUMAMD Human S-adenosylmethionine deca; 228_at M35416 IFEATURE=mRNA /DEFINITION=HUMRALBA Human GTP-binding protein (RALB; 167_at U49436 /FEATURE= /DEFINITION=HSU49436 Human translation initiation factor.

Metagene 303; 34525_at Cluster Incl. AB018563:Homo sapiens TML1 mRNA, complete cds /cds=(30,41; 38578_at Cluster Incl. M63928:Homo sapiens T cell activation antigen (CD27) mRNA; 38859_at Cluster Incl. AL080141:Homo sapiens mRNA; cDNA DKFZp434M183 (from clone; 40396_at Cluster Incl. U49395:Human ionotropic ATP receptor P2X5a mRNA, complete; 38968_at Cluster Incl. AB005047:Homo sapiens mRNA for SH3 binding protein, compl; 38760_f_at Cluster Incl. U90546:Human butyrophilin (BTF4) mRNA, complete cds /cd; 38786_at Cluster Incl. AL079279:Homo sapiens mRNA full length insert cDNA clone; 406_at X53587 /FEATURE=mRNA /DEFINITION=HSINTB4R Human mRNA for integrin beta 4.

Metagene 304; 35067_at Cluster Incl. AI925946:wh12f04.x1 Homo sapiens cDNA, 3 end /clone=IMAG; 35450_s_at Cluster Incl. AF015553:Homo sapiens TFII-I protein (TFII-I) mRNA, com; 35949_at Cluster Incl. AB018317:Homo sapiens mRNA for KIAA0774 protein, partial; 36258_at Cluster Incl. Y07512:Human mRNA for type 1 beta cGMP-dependent protein; 36791_g_at Cluster Incl. M19267:Human tropomyosin mRNA, complete cds /cds=(286,1; 41454_at Cluster Incl. W27949:39h3 Homo sapiens cDNA /gb=W27949 /gi=1307897 /ug=; 31867_at Cluster Incl. AF052174:Homo sapiens clone 24630 mRNA sequence /cds=UNKN; 32078_at Cluster Incl. D42055:Human mRNA for KIAA0093 gene, partial cds /cds=(0,; 33800_at Cluster Incl. AF036927:Homo sapiens adenylyl cyclase type IX mRNA, comp; 34202_at Cluster Incl. AL050071:Homo sapiens mRNA; cDNA DKFZp566B0846 (from clon; 34719_at Cluster Incl. AB020645:Homo sapiens mRNA for KIAA0838 protein, complete; 36029_at Cluster Incl. U57911:Human fetal brain (239FB) mRNA, from the WAGR regi; 36894_at Cluster Incl. AL031846:dJ742C19.5 (novel Chromobox protein) /cds=(89,84; 37225_at Cluster Incl. D79994:Human mRNA for KIAA0172 gene, partial cds /cds=(0,; 37253_at Cluster Incl. X92493:H. sapiens mRNA for STM-7 protein /cds=(419,2041) /; 37981_at Cluster Incl. D17530:Homo sapiens mRNA for drebrin E, complete cds /cds; 38656_s_at Cluster Incl. W27939:39g3 Homo sapiens cDNA /gb=W27939 /gi=1307887 /u; 39038_at Cluster Incl. AF093118:Homo sapiens UP50 mRNA, complete cds /cds=(168,1; 39388_at Cluster Incl. AA902713:ok71f11.s1 Homo sapiens cDNA, 3 end /clone=IMAG; 40064_at Cluster Incl. AB011121:Homo sapiens mRNA for KIAA0549 protein, partial; 41744_at Cluster Incl. AF070533:Homo sapiens clone 24619 mRNA sequence /cds=UNKN; 33404_at Cluster Incl. U02390:Human adenylyl cyclase-associated protein homolog; 33405_at Cluster Incl. N90755:zb22c08.s1 Homo sapiens cDNA, 3 end /clone=IMAGE-; 34356_at Cluster Incl. U52960:Human RNA polymerase II complex component SRB7 mRN; 34803_at Cluster Incl. AF022789:Homo sapiens ubiquitin hydrolyzing enzyme I (UBH; 35831_at Cluster Incl. AB014511:Homo sapiens mRNA for KIAA0611 protein, partial; 37655_at Cluster Incl. X75304:H. sapiens giantin mRNA /cds=(126,9905) /gb=X75304; 38032_at Cluster Incl. AB018279:Homo sapiens mRNA for KIAA0736 protein, complete; 38465_at Cluster Incl. M37721:Human peptidylglycine alpha-amidating monooxygenas; 38839_at Cluster Incl. AL096719:Homo sapiens mRNA; cDNA DKFZp566N043 (from clone; 38840_s_at Cluster Incl. L10678:Human profilin II mRNA, complete cds /cds=(13,43; 39178_at Cluster Incl. L10333:Homo sapiens neuroendocrine-specific protein A (NS; 39544_at Cluster Incl. AB002351:Human mRNA for KIAA0353 gene, partial cds /cds=(; 40966_at Cluster Incl. AF099989:Homo sapiens Ste-20 related kinase SPAK mRNA, co; 32531_at Cluster Incl. X52947:Human mRNA for cardiac gap junction protein /cds=(.

Metagene 305; 31667_r_at Cluster Incl. W27698:36f8 Homo sapiens cDNA /gb=W27698 /gi=1307664 /u; 31671_at Cluster Incl. D82351:Human retropseudogene MSSP-1 DNA, complete cds /cd; 32336_at Cluster Incl. X05236:Human fibroblast mRNA for aldolase A /cds=(146,124; 32929_at Cluster Incl. R56605:yg94a11.r1 Homo sapiens cDNA, 5 end /clone=IMAGE-; 38921_at Cluster Incl. U86078:Homo sapiens calmodulin-stimulated phosphodiestera; 39276_g_at Cluster Incl. M76558:Human neuronal DHP-sensitive, voltage-dependent,; 33311_at Cluster Incl. U05569:Human alphaA-crystallin (CRYA1) mRNA, complete cds; 36008_at Cluster Incl. AF041434:Homo sapiens potentially prenylated protein tyro; 37267_at Cluster Incl. Z50115:H. sapiens mRNA for thimet oligopeptidase (metallop; 38726_at Cluster Incl. W80399:zh49e04.s1 Homo sapiens cDNA, 3 end /clone=IMAGE-; 38997_at Cluster Incl. X96924:H. sapiens gene encoding mitochondrial citrate tran; 39406_at Cluster Incl. U50330:Human procollagen C-proteinase (pCP-2) mRNA, compl; 40116_at Cluster Incl. X15573:Human liver-type 1-phosphofructokinase (PFKL) mRNA; 32844_at Cluster Incl. AF104913:Homo sapiens eukaryotic protein synthesis initia; 33913_at Cluster Incl. M33509:Human HLA-B-associated transcript 2 (BAT2) mRNA, c; 37719_at Cluster Incl. AF070539:Homo sapiens clone 24433 myelodysplasia/myeloid; 41310_f_at Cluster Incl. X12794:Human v-erbA related ear-2 gene /cds=(0,1211) /g; 33129_at Cluster Incl. N80906:zb07c05.s1 Homo sapiens cDNA, 3 end /clone=IMAGE-; 1444_at AB003177 /FEATURE= /DEFINITION=AB003177 Homo sapiens mRNA for proteasome; 1395_at L25081 /FEATURE= /DEFINITION=HUMRHOCA Homo sapiens GTPase (rhoC) mRNA, c; 1306_at D12686 /FEATURE= /DEFINITION=HUMEIF4G Human mRNA for eukaryotic initiati; 1312_at D38047 /FEATURE= /DEFINITION=HUMPSP31 Human mRNA for 26S proteasome subu; 884_at M59911 /FEATURE= /DEFINITION=HUMINTA3A Human integrin alpha-3 chain mRNA,; 848_at U19261 /FEATURE= /DEFINITION=HSU19261 Homo sapiens Epstein-Barr virus-ind; 806_at U56998 /FEATURE= /DEFINITION=HSU56998 Human putative serine/threonine pro.

Metagene 306; 38177_at Cluster Incl. AJ001015:Homo sapiens mRNA encoding RAMP2 /cds=(68,595) /; 31904_at Cluster Incl. U67733:Human cGMP-stimulated 3,5-cyclic nucleotide phosp; 33295_at Cluster Incl. X85785:H. sapiens DARC gene /cds=(494,1510) /gb-X85785 /gi; 33790_at Cluster Incl. AI720438:as81g04.x1 Homo sapiens cDNA, 3 end /clone=IMAG; 34235_at Cluster Incl. AB018301:Homo sapiens mRNA for KIAA0758 protein, partial; 35147_at Cluster Incl. AB002360:Human mRNA for KIAA0362 gene, partial cds /cds=(; 37961_at Cluster Incl. U90907:Human clone 23907 mRNA sequence /cds=UNKNOWN /gb=U; 36174_at Cluster Incl. X70326:H. sapiens MacMarcks mRNA /cds=(13,600) /gb=X70326; 37042_at Cluster Incl. U09577:Homo sapiens lysosomal hyaluronidase (LUCA2 /HYAL2); 37394_at Cluster Incl. J03507:Human complement protein component C7 mRNA, comple; 37397_at Cluster Incl. L34657:Homo sapiens platelet/endothelial cell adhesion mo; 37398_at Cluster Incl. AA100961:zn40b06.s1 Homo sapiens cDNA, 3 end /clone=IMAG; 38453_at Cluster Incl. X15606:Human mRNA for ICAM-2, cell adhesion ligand for LF; 39135_at Cluster Incl. AB018310:Homo sapiens mRNA for KIAA0767 protein, partial; 41266_at Cluster Incl. X53586:Human mRNA for integrin alpha 6 /cds=UNKNOWN /gb=X; 2039_s_at M14333 /FEATURE= /DEFINITION=HUMCSYNA Homo sapiens c-syn protooncogene; 607_s_at M10321 /FEATURE=mRNA /DEFINITION=HUMVWFM Human von Willebrand factor mR; 590_at M32334 /FEATURE=cds /DEFINITION=HUMICAM4 Homo sapiens intercellular adhes; 268_at L34657 /FEATURE=mRNA /DEFINITION=HUMPECAM27 Homo sapiens platelet/endothe.

Metagene 307; 36374_at Cluster Incl. U79293:Human clone 23948 mRNA sequence /cds=UNKNOWN /gb=U; 33577_at Cluster Incl. AC004079:Homo sapiens PAC clone DJ0167F23 from 7p15 /cds=; 38916_at Cluster Incl. U46023:Human Xq28 mRNA, complete cds /cds=(283,2388) /gb=; 39243_s_at Cluster Incl. U94319:Human autoantigen DFS70 mRNA, partial cds /cds=(; 40354_at Cluster Incl. AB023421:Homo sapiens mRNA for heat shock protein apg-1,; 41049_at Cluster Incl. S62539:insulin receptor substrate-1 [human, skeletal musc; 41476_at Cluster Incl. N36926:yy38e04.s1 Homo sapiens cDNA, 3 end /clone=IMAGE-; 41649_at Cluster Incl. AF038177:Homo sapiens clone 23899 mRNA sequence /cds=UNKN; 41859_at Cluster Incl. AB020316:Homo sapiens mRNA for dermatan/chondroitin sulfa; 32044_at Cluster Incl. D13635:Human mRNA for KIAA0010 gene, complete cds /cds=(3; 34180_at Cluster Incl. AB002292:Human mRNA for KIAA0294 gene, complete cds /cds=; 35202_at Cluster Incl. AF025654:Homo sapiens mRNA capping enzyme (HCE) mRNA, com; 35697_at Cluster Incl. L76259:Homo sapiens PTS gene, complete cds /cds=(68,505); 36463_at Cluster Incl. AB020680:Homo sapiens mRNA for KIAA0873 protein, partial; 36526_at Cluster Incl. AF000416:Homo sapiens EXT-like protein 2 (EXTL2) mRNA, co; 36544_at Cluster Incl. AF038193:Homo sapiens clone 23608 mRNA sequence /cds=UNKN; 36554_at Cluster Incl. Y15521:Homo sapiens ASMTL gene /cds=(0,1889) /gb=Y15521 /; 36864_at Cluster Incl. AJ001625:Homo sapiens mRNA for Pex3 protein /cds=(63,1184; 38335_at Cluster Incl. U88620:Human 8-hydroxyguanine glycosylase (hMMH) mRNA, co; 38692_at Cluster Incl. AF045451:Homo sapiens transcriptional regulatory protein; 39380_at Cluster Incl. AB014597:Homo sapiens mRNA for KIAA0697 protein, partial; 39405_at Cluster Incl. D87455:Human mRNA for KIAA0266 gene, complete cds /cds=(7; 39701_at Cluster Incl. AB006625:Homo sapiens mRNA for KIAA0287 gene, partial cds; 4011_g_at Cluster Incl. U49283:Human NAD+-specific isocitrate dehydrogenase bet; 40838_at Cluster Incl. AB011102:Homo sapiens mRNA for KIAA0530 protein, partial; 32842_at Cluster Incl. X89984:H. sapiens mRNA for BCL7A protein /cds=(953,1648) /; 33355_at Cluster Incl. AL049381:Homo sapiens mRNA; cDNA DKFZp586J2118 (from clon; 33357_at Cluster Incl. AB011159:Homo sapiens mRNA for KIAA0587 protein, complete; 33397_at Cluster Incl. AL050383:Homo sapiens mRNA; cDNA DKFZp547E0510 (from clon; 33399_at Cluster Incl. AA142942:zl43c04.s1 Homo sapiens cDNA, 3 end /clone=IMAG; 34808_at Cluster Incl. AB023216:Homo sapiens mRNA for KIAA0999 protein, partial; 35316_at Cluster Incl. U41654:Human adenovirus protein E3-14.7k interacting prot; 35787_at Cluster Incl. AI986201:wr81a01.x1 Homo sapiens cDNA, 3 end /clone=IMAG; 36196_at Cluster Incl. U24183:Human phosphofructokinase (PFKM) mRNA, complete cd; 40537_at Cluster Incl. AB018284:Homo sapiens mRNA for KIAA0741 protein, complete; 41338_at Cluster Incl. AI951946:wx39f10.x1 Homo sapiens cDNA, 3 end /clone=IMAG; 33186_i_at Cluster Incl. AL046961:DKFZp586J0417_r1 Homo sapiens cDNA, 5 end /cl; 1828_s_at M27968 /FEATURE=mRNA /DEFINITION=HUMFGFB Human basic fibroblast growth; 786_at X06745 /FEATURE=mRNA /DEFINITION=HSPOLAR Human mRNA for DNA polymerase a1; 202_at M65217 /FEATURE= /DEFINITION=HUMHSF2 Human heat shock factor 2 (HSF2) mRN; 185_at U04840 /FEATURE= /DEFINITION=HSU04840 Human onconeural ventral antigen-1.

Metagene 308; 31979_at Cluster Incl. D49818:Homo sapiens mRNA for 6-phosphofructo-2-kinase/fru; 34041_at Cluster Incl. U83171:Human macrophage-derived chemokine precursor (MDC); 39977_at Cluster Incl. U69274:Human zinc finger protein mRNA, complete cds /cds=; 41752_at Cluster Incl. W28190:43c6 Homo sapiens cDNA /gb=W28190 /gi=1308138 /ug=; 35302_at Cluster Incl. AJ132712:Homo sapiens mRNA for tip associating protein (T; 40944_at Cluster Incl. AF028008:Homo sapiens SP1-like zinc finger transcription; 1909_at M14745 /FEATURE= /DEFINITION=HUMBCL2C Human bcl-2 mRNA.

Metagene 309; 36393_at Cluster Incl. U43843:Human h-neuro-d4 protein mRNA, complete cds /cds=(; 36310_at Cluster Incl. X86570:Homo sapiens mRNA for acidic hair keratin 1 /cds=(; 33707_at Cluster Incl. AF058921:Homo sapiens cytosolic phospholipase A2-gamma mR; 37186_s_at Cluster Incl. U11863:Human clone HP-DAO2 diamine oxidase, copper/topa; 37226_at Cluster Incl. U15172:Homo sapiens BCL2 /adenovirus E1B 19 kD-interacting; 41130_at Cluster Incl. Y08698:H. sapiens mRNA for RanBP3 (59 kDa) /cds=(20,1708); 34847_s_at Cluster Incl. AF112471:Homo sapiens calcium/calmodulin-dependent prot; 36184_at Cluster Incl. L06419:Homo sapiens lysyl hydroxylase (PLOD) mRNA, comple; 40219_at Cluster Incl. AI796944:we25b03.x1 Homo sapiens cDNA, 3 end /clone=IMAG; 847_at U17969 /FEATURE=exons#1-6 /DEFINITION=HSU17969 Human initiation factor eI.

Metagene 310; 32394_s_at Cluster Incl. X55954:Human mRNA for HL23 ribosomal protein homologue; 33458_r_at Cluster Incl. AI688098:wc92f08.x1 Homo sapiens cDNA, 3 end /clone=IM; 37119_at Cluster Incl. AL035296:H. sapiens gene from PAC 560B9 /cds=(139,564) /gb; 39260_at Cluster Incl. U59185:Human putative monocarboxylate transporter (MCT) m; 31850_at Cluster Incl. M90656:Human gamma-glutamylcysteine synthetase (GCS) mRNA; 31869_at Cluster Incl. AB014540:Homo sapiens mRNA for KIAA0640 protein, partial; 32624_at Cluster Incl. AL050050:Homo sapiens mRNA; cDNA DKFZp566D133 (from clone; 34657_at Cluster Incl. AB014529:Homo sapiens mRNA for KIAA0629 protein, partial; 34688_at Cluster Incl. AB029001:Homo sapiens mRNA for KIAA1078 protein, partial; 34752_at Cluster Incl. AL080111:Homo sapiens mRNA; cDNA DKFZp586G2222 (from clon; 34753_at Cluster Incl. X92396:H. sapiens mRNA for novel gene in Xq28 region /cds=; 34761_r_at Cluster Incl. U41766:Human metalloprotease/disintegrin/cysteine-rich; 34763_at Cluster Incl. AF020043:Homo sapiens chromosome-associated polypeptide (; 35187_at Cluster Incl. AL080216:Homo sapiens mRNA; cDNA DKFZp586K1123 (from clon; 36913_at Cluster Incl. U75679:Human histone stem-loop binding protein (SLBP) mRN; 37638_at Cluster Incl. D50857:Human DOCK180 protein mRNA, complete cds /cds=(23,; 37962_r_at Cluster Incl. D63506:Homo sapiens mRNA for unc-18homologue, complete; 37973_at Cluster Incl. AB018256:Homo sapiens mRNA for KIAA0713 protein, partial; 37994_at Cluster Incl. X69962:H. sapiens FMR-1 mRNA /cds=(219,2117) /gb=X69962 /g; 38654_at Cluster Incl. X65488:H. sapiens U21.1 mRNA /cds=(41,2461) /gb=X65488 /gi; 38992_at Cluster Incl. X64229:H. sapiens dek mRNA /cds=(33,1160) /gb=X64229 /gi=3; 38993_r_at Cluster Incl. W27522:32a12 Homo sapiens cDNA /gb=W27522 /gi=1307326 /; 39028_at Cluster Incl. Y08890:H. sapiens mRNA for Ran_GTP binding protein 5 /cds=; 39046_at Cluster Incl. AL049324:Homo sapiens mRNA; cDNA DKFZp564D246 (from clone; 39071_at Cluster Incl. M14648:Human cell adhesion protein (vitronectin) receptor; 39344_at Cluster Incl. U53209:Human transformer-2 alpha (htra-2 alpha) mRNA, com; 39389_at Cluster Incl. M38690:Human CD9 antigen mRNA, complete cds /cds=(51,737); 39714_at Cluster Incl. AF042081 Homo sapiens SH3 domain binding glutamic acid-ri; 40048_at Cluster Incl. D43951:Human mRNA for KIAA0099 gene, complete cds /cds=(5; 40125_at Cluster Incl. L10284:Homo sapiens integral membrane protein, calnexin,; 40509_at Cluster Incl. J04058:Human electron transfer flavoprotein alpha-subunit; 41132_r_at Cluster Incl. U01923:Human BTK region clone ftp-3 mRNA /cds=UNKNOWN /; 41738_at Cluster Incl. M64110:Human caldesmon mRNA, complete cds /cds=(111,1727); 32175_at Cluster Incl. S72008:hCDC10=CDC10 homolog [human, fetal lung, mRNA, 231; 32219_at Cluster Incl. D50927:Human mRNA for KIAA0137 gene, complete cds /cds=(1; 32778_at Cluster Incl. D26070:Human mRNA for type 1 inositol 1,4,5-trisphosphate; 32803_at Cluster Incl. AF104398:Homo sapiens comichon mRNA, complete cds /cds=(; 32841_at Cluster Incl. U19765:Human nucleic acid binding protein gene, complete; 32850_at Cluster Incl. Z25535:H. sapiens mRNA for nuclear pore complex protein hn; 33443_at Cluster Incl. Z99129:Human DNA sequence from clone 425C14 on chromosome; 33859_at Cluster Incl. U96915:Homo sapiens sin3 associated polypeptide p18 (SAP1; 34306_at Cluster Incl. AB007888:Homo sapiens KIAA0428 mRNA, complete cds /cds=(1; 34326_at Cluster Incl. X82103:H. sapiens mRNA for beta-COP /cds=(0,911) /gb=X8210; 34329_at Cluster Incl. N25547:yx76e06.s1 Homo sapiens cDNA, 3 end /clone=IMAGE-; 34368_at Cluster Incl. U31814:Human transcriptional regulator homolog RPD3 mRNA,; 34402_at Cluster Incl. AB024327:Homo sapiens pt-wd mRNA for WD-40 repeat protein; 34793_s_at Cluster Incl. M22299:Human T-plastin polypeptide mRNA, complete cds,; 34814_at Cluster Incl. AL041443:DKFZp434D0717_s1 Homo sapiens cDNA, 3 end /clon; 34850_at Cluster Incl. AB017644:Homo sapiens mRNA for ubiquitin-conjugating enzy; 34887_at Cluster Incl. N92548:zb29g04.s1 Homo sapiens cDNA, 3 end /clone=IMAGE-; 35747_at Cluster Incl. AF035287:Homo sapiens clone 23742 mRNA, partial cds /cds=; 35754_at Cluster Incl. L40391:Homo sapiens (clone s153) mRNA fragment /cds=UNKNO; 35790_at Cluster Incl. AF054179:Homo sapiens H beta 58 homolog mRNA, complete cd; 35814_at Cluster Incl. AF064603:Homo sapiens GA17 protein mRNA, complete cds /cd; 36107_at Cluster Incl. AA845575:ak04e09.s1 Homo sapiens cDNA, 3 end /clone=IMAG; 36112_r_at Cluster Incl. X75755:H. sapiens PR264 gene /cds=(109,774) /gb=X75755 /; 36159_s_at Cluster Incl. U29185:Homo sapiens prion protein (PrP) gene, complete; 36163_at Cluster Incl. L13761:Human dihydrolipoamide dehydrogenase gene /cds=(20; 36606_at Cluster Incl. X51405:Human mRNA for carboxypeptidase E (EC 3.4.17.10) /; 36631_at Cluster Incl. D49396:Human mRNA for Apo1_Human (MER5(Aop1-Mouse)-like p; 36654_s_at Cluster Incl. M29065:Human hnRNP A2 protein mRNA /cds=(155,1180) /gb=; 36688_at Cluster Incl. U11313:Human sterol carrier protein-X/sterol carrier prot; 36971_at Cluster Incl. D87446:Human mRNA for KIAA0257 gene, partial cds /cds=(0,; 36980_at Cluster Incl. U03105:Human B4-2 protein mRNA, complete cds /cds=(113,10; 36981_at Cluster Incl. AF070649:Homo sapiens clone 24452 mRNA sequence /cds=UNKN; 36998_s_at Cluster Incl. Y08262:H. sapiens mRNA for SCA2 protein /cds=(0,2746) /g; 37000_at Cluster Incl. AL035304:H. sapiens gene from PAC 295C6, similar to rat PO; 37358_at Cluster Incl. AI039880:ox97c12.x1 Homo sapiens cDNA, 3 end /clone=IMAG; 37374_at Cluster Incl. M82809:Human annexin IV (ANX4) mRNA, complete cds /cds=(7; 37696_at Cluster Incl. L06328:Human voltage-dependent anion channel isoform 2 (V; 37708_r_at Cluster Incl. M81118:Human alcohol dehydrogenase chi polypeptide (ADH; 37731_at Cluster Incl. Z29064:H. sapiens AF-1p mRNA /cds=(92,2782) /gb=Z29064 /gi; 37740_r_at Cluster Incl. J02683:Human ADP/ATP carrier protein mRNA, complete cds; 38079_at Cluster Incl. AL049367:Homo sapiens mRNA; cDNA DKFZp586B0918 (from clon; 38106_at Cluster Incl. AJ012409:Homo sapiens mRNA for hypothetical protein, clon; 38114_at Cluster Incl. D38551:Human mRNA for KIAA0078 gene, complete cds /cds=(1; 39471_at Cluster Incl. Z48042:H. sapiens mRNA encoding GPI-anchored protein p137; 41257_at Cluster Incl. D16217:Human mRNA for calpastatin, complete cds /cds=(162; 32517_at Cluster Incl. AB028639:Homo sapiens CAPN7 mRNA for PalBH, complete cds; 1848_at M22995 /FEATURE= /DEFINITION=HUMKREV1A Human ras-related protein (Krev-1; 1659_s_at D78132 /FEATURE= /DEFINITION=D78132 Homo sapiens mRNA for ras-related; 1064_at U02680 /FEATURE= /DEFINITION=HSU02680 Human protein tyrosine kinase mRNA; 818_s_at U72936 /FEATURE= /DEFINITION=HSU72936 Homo sapiens putative DNA depende; 339_at AF035752 /FEATURE= /DEFINITION=AF035752 Homo sapiens caveolin-2 mRNA, com; 324_f_at Transcription Factor Btf3b.

Metagene 311; 37483_at Cluster Incl. AB018287:Homo sapiens mRNA for KIAA0744 protein, complete; 39673_i_at Cluster Incl. AB011792:Homo sapiens mRNA for extracellular matrix pro; 36054_at Cluster Incl. AB020699:Homo sapiens mRNA for KIAA0892 protein, partial; 39432_at Cluster Incl. AF038662:Homo sapiens chromosome 3q13 beta-1,4-galactosyl; 40424_at Cluster Incl. AI017935:ou43h10.x1 Homo sapiens cDNA, 3 end /clone=IMAG; 40814_at Cluster Incl. L40586:Homo sapiens iduronate-2-sulphatase (IDS) mRNA, co; 41201_at Cluster Incl. AF000430:Homo sapiens dynamin-like protein mRNA, complete; 41766_at Cluster Incl. D55649:Human mRNA for alpha mannosidase II isozyme, compl; 35781_g_at Cluster Incl. AF035292:Homo sapiens clone 23584 mRNA sequence /cds=UN; 36659_at Cluster Incl. X05610:Human mRNA for type IV collagen alpha (2) chain /c; 40957_at Cluster Incl. D63881:Human mRNA for KIAA0160 gene, partial cds /cds=(0,; 41788_i_at Cluster Incl. AB014569:Homo sapiens mRNA for KIAA0669 protein, comple.

Metagene 312; 31506_s_at Cluster Incl. L12691:Human neutrophil peptide-3 gene, complete cds /c; 38585_at Cluster Incl. M91036:H. sapiens G-gamma globin and A-gamma globin genes,; 39208_i_at Cluster Incl. M54995:Human connective tissue activation peptide III m; 41096_at Cluster Incl. AI126134:qd77c05.x1 Homo sapiens cDNA, 3 end /clone=IMAG; 41617_at Cluster Incl. AI349593:qp73f07.x1 Homo sapiens cDNA, 3 end /clone=IMAG.

Metagene 313; 41858_at Cluster Incl. AL049261:Homo sapiens mRNA; cDNA DKFZp564E053 (from clone; 36036_at Cluster Incl. J05500:Human beta-spectrin (SPTB) mRNA, complete cds /cds; 36081_s_at Cluster Incl. AB004848:Homo sapiens mRNA expressed in placenta, clone; 38621_at Cluster Incl. AJ012008:Homo sapiens genes encoding RNCC protein, DDAH p; 37716_at Cluster Incl. X05323:Human MRC OX-2 gene signal sequence /cds=(0,824) /; 39103_s_at Cluster Incl: H98552:yv97h03.s1 Homo sapiens cDNA, 3 end /clone=IMAG; 589_at M32313 /FEATURE=mRNA /DEFINITION=HUM5AR Human steroid 5-alpha-reductase m.

Metagene 314; 35071_s_at Cluster Incl. AF042377:Homo sapiens GDP-mannose 4,6 dehydratase mRNA,; 32313_at Cluster Incl. M12125:Human fibroblast muscle-type tropomyosin mRNA, com; 32314_g_at Cluster Incl. M12125:Human fibroblast muscle-type tropomyosin mRNA, c; 34428_at Cluster Incl. D50312:Human mRNA for uKATP-1, complete cds /cds=(270,154; 38881_i_at Cluster Incl. AF096870:Homo sapiens estrogen-responsive B box protein; 40039_g_at Cluster Incl. W02490:za48b02.r1 Homo sapiens cDNA, 5 end /clone=IMAG; 41396_at Cluster Incl. AB006629:Homo sapiens mRNA for KIAA0291 gene, partial cds; 41412_at Cluster Incl. AL023553:dJ347H13.2 (human ortholog of rat Pippin) /cds=(; 31830_s_at Cluster Incl. Y13492:Homo sapiens mRNA for smoothelin-B /cds=(219,297; 31831_at Cluster Incl. AI888563:wn33a05.x1 Homo sapiens cDNA, 3 end /clone=IMAG; 32104_i_at Cluster Incl. U66063:Homo sapiens calcium/calmodulin-dependent protei; 32133_at Cluster Incl. AB011161:Homo sapiens mRNA for KIAA0589 protein, partial; 32625_at Cluster Incl. X15357:Human mRNA for natriuretic peptide receptor (ANP-A; 34724_at Cluster Incl. AI670100:wc11g10.x1 Homo sapiens cDNA, 3 end /clone=IMAG; 35146_at Cluster Incl. AB007836:Homo sapiens mRNA for Hic-5, partial cds /cds=(0; 35239_at Cluster Incl. X86810:Homo sapiens EDMD gene /cds=(110,874) /gb=X86810 /; 36471_f_at Cluster Incl. U46746:Human dystrobrevin-epsilon mRNA, complete cds /c; 36892_at Cluster Incl. AF032108:Homo sapiens integrin alpha-7 mRNA, complete cds; 36900_at Cluster Incl. U52426:Homo sapiens GOK (STIM1) mRNA, complete cds /cds=(; 37256_at Cluster Incl. AI829890:wj47a06.x1 Homo sapiens cDNA, 3 end /clone=IMAG; 37567_at Cluster Incl. X98834:H. sapiens mRNA for zinc finger protein, Hsa12 /cds; 38338_at Cluster Incl. AI201108:qf69g07.x1 Homo sapiens cDNA, 3 end /clone=IMAG; 40109_at Cluster Incl. J03161:Human serum response factor (SRF) mRNA, complete c; 33883_at Cluster Incl. AB001466:Homo sapiens mRNA for Efs1, complete cds /cds=(6; 33912_at Cluster Incl. Y13834:Homo sapiens mRNA for farnesylated-proteins conver; 34320_at Cluster Incl. AL050224:Homo sapiens mRNA; cDNA DKFZp586L2123 (from clon; 34802_at Cluster Incl. X15882:Human mRNA for collagen VI alpha-2 C-terminal glob; 35336_at Cluster Incl. AL021707:Human DNA sequence from clone 508115 on chromoso; 35846_at Cluster Incl. M24899:Human triiodothyronine (ear7) mRNA, complete cds /; 36156_at Cluster Incl. U41518:Human channel-like integral membrane protein (AQP-; 37300_at Cluster Incl. AB002323:Human mRNA for KIAA0325 gene, partial cds /cds=(; 38437_at Cluster Incl. X80199:H. sapiens MLN51 mRNA /cds=(233,1837) /gb=X80199 /g; 39108_at Cluster Incl. U22526:Human 2,3-oxidosqualene-lanosterol cyclase mRNA, c; 39145_at Cluster Incl. J02854:Human 20-kDa myosin light chain (MLC-2) mRNA, comp; 39548_at Cluster Incl. U77970:Human neuronal PAS2 (NPAS2) mRNA, complete cds /cd; 40174_at Cluster Incl. AF026004:Homo sapiens chloride channel protein (CIC-2) mR; 40562_at Cluster Incl. M69013:Human guanine nucleotide-binding regulatory protei; 41282_s_at Cluster Incl. AA194159:zr37h01.r1 Homo sapiens cDNA, 5 end /clone=IM; 823_at U84487 /FEATURE= /DEFINITION=HSU84487 Human CX3C chemokine precursor, mRN; 361_at Y13620 /FEATURE= /DEFINITION=HSRNABCL9 Homo sapiens mRNA for BCL9 gene.

Metagene 315; 33697_at Cluster Incl. Y12851:Homo sapiens P2X7 gene, exon 1 and joined CDS /cds; 32329_at Cluster Incl. X99142:H. sapiens mRNA for hair keratin, hHb6 /cds=(0,1451; 37796_at Cluster Incl. AF053356:Homo sapiens chromosome 7q22 sequence /cds=(0,24; 38132_at Cluster Incl. M88338:Human serum constituent protein (MSE55) mRNA, comp; 40687_at Cluster Incl. M96789:Homo sapiens connexin 37 (GJA4) mRNA, complete cds; 40733_f_at Cluster Incl. D89377:Homo sapiens mRNA for MSX-2, complete cds /cds=(; 41428_at Cluster Incl. AF104942:Homo sapiens ABC transporter MOAT-C (MOAT-C) mRN; 41856_at Cluster Incl. AL049370:Homo sapiens mRNA; cDNA DKFZp586D0918 (from clon; 34294_at Cluster Incl. AL041493:DKFZp434F2117_s1 Homo sapiens cDNA, 3 end /clon; 37281_at Cluster Incl. D87071:Human mRNA for KIAA0233 gene, complete cds /cds=(2; 37573_at Cluster Incl. AF007150:Homo sapiens clone 23767 and 23782 mRNA sequence; 41180_i_at Cluster Incl. U49785:Human D-dopachrome tautomerase mRNA, complete cd; 32201_at Cluster Incl. Z96932:Homo sapiens mRNA for NA14 protein /cds=(46,405) /; 34885_at Cluster Incl. AJ002308:Homo sapiens mRNA for synaptogyrin 2 /cds=(29,70; 35256_at Cluster Incl. AL096737:Homo sapiens mRNA; cDNA DKFZp434F152 (from clone; 35369_at Cluster Incl. AB023154:Homo sapiens mRNA for KIAA0937 protein, partial; 36987_at Cluster Incl. M94362:Human lamin B2 (LAMB2) mRNA, partial cds /cds=(0,1; 37049_g_at Cluster Incl. U58970:Human putative outer mitochondrial membrane 34 k; 37384_at Cluster Incl. D13640:Human mRNA for KIAA0015 gene, complete cds /cds=(1; 39166_s_at Cluster Incl. D83174:Human mRNA for collagen binding protein 2, compl; 39831_at Cluster Incl. AI972631:wr41c07.x1 Homo sapiens cDNA, 3 end /clone=IMAG; 40183_at Cluster Incl. AI660225:we68f05.x1 Homo sapiens cDNA, 3 end /clone=IMAG; 1083_s_at M35093 /FEATURE=cds /DEFINITION=HUMETMAGA Human secreted epithelial tu; 749_at D78586 /FEATURE= /DEFINITION=HUMMUPCAD Human CAD mRNA for multifunctional.

Metagene 316; 33749_at Cluster Incl. AB007455:Homo sapiens mRNA for P53TG1-A, complete cds /cd; 35631_at Cluster Incl. U37689:Human RNA polymerase II subunit (hsRPB8) mRNA, com; 37184_at Cluster Incl. L37792:Human syntaxin 1A mRNA, complete cds /cds=(1,867); 34298_at Cluster Incl. X69532:H. sapiens gene for inter-alpha-trypsin inhibitor h; 37333_at Cluster Incl. X63692:H. sapiens mRNA for DNA (cytosin-5)-methyltransfera; 39469_s_at Cluster Incl. W25905:14g5 Homo sapiens cDNA /gb=W25905 /gi=1306028 /u; 41361_at Cluster Incl. W28148:43f2 Homo sapiens cDNA /gb=W28148 /gi=1308159 /ug=; 1833_at M68520 /FEATURE= /DEFINITION=HUMCDC2A Human cdc2-related protein kinase; 1622_at D87116 /FEATURE= /DEFINITION=D87116 Human mRNA for MAP kinase kinase 3b.

Metagene 317; 35374_at Cluster Incl. AB007914:Homo sapiens mRNA for KIAA0445 protein, complete; 37836_at Cluster Incl. AB023215:Homo sapiens mRNA for KIAA0998 protein, partial; 41120_at Cluster Incl. D14686:Human gene for glycine cleavage system T-protein /; 41865_at Cluster Incl. AF052185:Homo sapiens clone 24418 mRNA sequence /cds=UNKN; 31801_at Cluster Incl. AI808712:wf57c05.x1 Homo sapiens cDNA, 3 end /clone=IMAG; 37555_at Cluster Incl. X95263:H. sapiens mRNA for PWP2 protein /cds=(31,2790) /gb; 41172_at Cluster Incl. AA126515:zn85c12.s1 Homo sapiens cDNA, 3 end /clone=IMAG; 35840_at Cluster Incl. AL050060:Homo sapiens mRNA; cDNA DKFZp566H073 (from clone; 36935_at Cluster Incl. M23379:Human GTPase-activating protein ras p21 (RASA) mRN; 38386_r_at Cluster Incl. U34683:Human glutathione synthetase mRNA, complete cds; 33161_at Cluster Incl. AI018098:ov65b11.s1 Homo sapiens cDNA, 3 end /clone=IMAG; 1373_at M31523 /FEATURE= /DEFINITION=HUMTFAA Human transcription factor (E2A) mR.

Metagene 318; 32405_at Cluster Incl. AB014607:Homo sapiens mRNA for KIAA0707 protein, partial; 33993_at Cluster Incl. M22919:Human nonmuscle/smooth muscle alkali myosin light; 40752_at Cluster Incl. AI024359:ov75g05.x1 Homo sapiens cDNA, 3 end /clone=IMAG; 32109_at Cluster Incl. AA524547:ng45h04.s1 Homo sapiens cDNA, 3 end /clone=IMAG; 32123_at Cluster Incl. L02870:Human alpha-1 type VII collagen (COL7A1) mRNA, com; 36480_at Cluster Incl. X80497:H. sapiens PHKLA mRNA /cds=(126,3833) /gb=X80497 /g; 40451_at Cluster Incl. AL080203:Homo sapiens mRNA; cDNA DKFZp434F222 (from clone; 40862_i_at Cluster Incl. X15334:Human gene for creatine kinase B (EC 2.7.3.2) /c; 40863_r_at Cluster Incl. X15334:Human gene for creatine kinase B (EC 2.7.3.2) /c; 32169_at Cluster Incl. AB020682:Homo sapiens mRNA for KIAA0875 protein, partial; 33822_at Cluster Incl. Z11584:H. sapiens mRNA for NuMA protein /cds=(258,6563) /g; 34827_at Cluster Incl. AF045458:Homo sapiens serine/threonine kinase ULK1 (ULK1); 36124_at Cluster Incl. X59434:Human rohu mRNA for rhodanese /cds=(34,924) /gb=X5; 37330_at Cluster Incl. U24266:Human pyrroline-5-carboxylate dehydrogenase (P5CDh; 37331_g_at Cluster Incl. U24266:Human pyrroline-5-carboxylate dehydrogenase (P5C; 37369_s_at Cluster Incl. L41066:Homo sapiens NF-AT3 mRNA, complete cds /cds=(141; 37405_at Cluster Incl. U29091:Human selenium-binding protein (hSBP) mRNA, comple; 37745_s_at Cluster Incl. U15780:Human p82 (ST5) mRNA, alternatively spliced, com; 38063_at Cluster Incl. U00952:Human clone A9A2BRB7 (CAC)n/(GTG)n repeat-containi; 33118_at Cluster Incl. U73167:Homo sapiens cosmid clone LUCA14 from 3p21.3 /cds=; 844_at U48707 /FEATURE= /DEFINITION=HSU48707 Human protein phosphatase-1 inhibit; 329_s_at Nuclear Mitotic Apparatus Protein 1, Alt. Splice Form 2.

Metagene 319; 32385_at Cluster Incl. AL050032:Homo sapiens mRNA; cDNA DKFZp566F1224 (from clon; 36337_at Cluster Incl. AI760801:wi67f11.x1 Homo sapiens cDNA, 3 end /clone=IMAG; 32921_at Cluster Incl. X83300:H. sapiens SMA4 mRNA /cds=(66,488) /gb=X83300 /gi=6; 37797_at Cluster Incl. AJ224326:Homo sapiens mRNA for putative ribulose-5-phosph; 41088_at Cluster Incl. X12433:Human pHS1-2 mRNA with ORF homologous to membrane; 31853_at Cluster Incl. AF080227:Homo sapiens embryonic ectoderm development prot; 33229_at Cluster Incl. U08316:Human insulin-stimulated protein kinase 1 (ISPK-1); 37537_at Cluster Incl. L04510:Human nucleotide binding protein mRNA, complete cd; 38637_at Cluster Incl. L16895:Human lysyl oxidase (LOX) gene, exon 7 /cds=(0,6); 1532_g_at U50535 /FEATURE= /DEFINITION=HSU50535 Human BRCA2 region, mRNA sequenc; 877_at M27691 /FEATURE= /DEFINITION=HUMCREB Human transactivator protein (CREB).

Metagene 320; 34714_at Cluster Incl. AL050267:Homo sapiens mRNA; cDNA DKFZp564A032 (from clone; 34721_at Cluster Incl. U42031:Human 54 kDa progesterone receptor-associated immu; 40504_at Cluster Incl. AF001601:Homo sapiens paraoxonase (PON2) mRNA, complete c; 40522_at Cluster Incl. X59834:Human rearranged mRNA for glutamine synthase /cds=; 36626_at Cluster Incl. X87176:H. sapiens mRNA for 17-beta-hydroxysteroid dehydrog; 36630_at Cluster Incl. Z50781:H. sapiens mRNA for leucine zipper protein /cds=(13; 1269_at M61906 /FEATURE= /DEFINITION=HUMP13KIN Human P13-kinase associated p85 m; 723_s_at Small Nuclear Ribonucleoprotein, Polypeptide C, Alt. Splice 2.

Metagene 321; 32497_s_at Cluster Incl. S70609:glycine transporter type 1b [human, substantia n; 34652_at Cluster Incl. U77968:Human neuronal PAS1 (NPAS1) mRNA, complete cds /cd; 35944_at Cluster Incl. AL031228:dJ1033B10.3 (GalT3 (beta3-Galactosyltransferase); 40843_at Cluster Incl. AF012023:Homo sapiens integrin cytoplasmic domain associa; 856_at S79267 /FEATURE= /DEFINITION=S79267 CD4 receptor {exons 1 and 2} [human,; 744_at D50487 /FEATURE= /DEFINITION=HUMHRH1 Human mRNA for RNA helicase (HRH1),; 453_at U66616 /FEATURE= /DEFINITION=HSU66616 Human SWI/SNF complex 170 KDa subun.

Metagene 322; 41810_at Cluster Incl. AA203545:zx59a05.r1 Homo sapiens cDNA, 5 end /clone=IMAG; 1863_s_at U67092 /FEATURE=exon#5 /DEFINITION=HSU67092 Human ataxia-telangiectasi; 1049_g_at U38480 /FEATURE= /DEFINITION=HSU38480 Human retinoid X receptor-gamma.

Metagene 323; 32306_g_at Cluster Incl. J03464:Human collagen alpha-2 type I mRNA, complete cds; 34916_s_at Cluster Incl. S76792:OX40=cell surface antigen [human, mRNA Partial,; 35879_at Cluster Incl. M77140:H. sapiens pro-galanin mRNA, 3 end /cds=(0,323) /g; 36231_at Cluster Incl. AC002073:Human PAC clone DJ515N1 from 22q11.2-q22 /cds=(0; 41872_at Cluster Incl. AF073308:Homo sapiens nonsyndromic hearing impairment pro; 31859_at Cluster Incl. J05070:Human type IV collagenase mRNA, complete cds /cds=; 33245_at Cluster Incl. AF004709:Homo sapiens stress-activated protein kinase 4 m; 37233_at Cluster Incl. AF079167:untitled /cds=(61,882) /gb=AF079167 /gi=4050003; 35816_at Cluster Incl. U46692:Human cystatin B gene, complete cds /cds=(96,392); 36575_at Cluster Incl. S59049:BL34=B cell activation gene [human, mRNA, 1398 nt]; 36657_at Cluster Incl. AA883870:am26f01.s1 Homo sapiens cDNA, 3 end /clone=IMAG; 38466_at Cluster Incl. X82153:H. sapiens mRNA for cathepsin O /cds=(129,1118) /gb; 1481_at L23808 /FEATURE /DEFINITION=HUMHME Human metalloproteinase (HME) mRNA,; 1482_g_at L23808 /FEATURE= /DEFINITION=HUMHME Human metalloproteinase (HME) mRNA; 973_at Y10032 /FEATURE=cds /DEFINITION=HSRNASTPK H. sapiens mRNA for putative ser; 980_at AF002020 /FEATURE= /DEFINITION=AF002020 Homo sapiens Niemann-Pick C disea; 649_s_at L06797 /FEATURE= /DEFINITION=HUMGPCR Human (clone L5) orphan G protein-; 668_s_at L22524 /FEATURE=expanded-cds /DEFINITION=HUMMATRY06 Human matrilysin ge; 432_s_at X02883 /FEATURE=cds /DEFINITION=HSTCRAC Human gene for T-cell receptor; 212_at M97639 /FEATURE= /DEFINITION=HUMROR2A Human transmembrane receptor (ror2); 128_at X82153 /FEATURE=cds /DEFINITION=HSOC2RNA H. sapiens mRNA for cathepsin 0.

Metagene 324; 33646_g_at Cluster Incl. X61094:H. sapiens RNA for GM2-activator protein (clone p; 34506_at Cluster Incl. M13928:Human delta-aminolevulinate dehydratase mRNA, comp; 41062_at Cluster Incl. AA037278:zc52c04.r1 Homo sapiens cDNA, 5 end /clone=IMAG; 520_at U07358 /FEATURE= /DEFINITION=HSU07358 Human protein kinase (zpk) mRNA, co.

Metagene 325; 31502_at Cluster Incl. W27953:39h7 Homo sapiens cDNA /gb=W27953 /gi=1307901 /ug=; 33069_f_at Cluster Incl. U06641:Human UDP glucuronosyltransferase mRNA, partial; 33080_s_at Cluster Incl. AB007943:Homo sapiens mRNA for KIAA0474 protein, comple; 34634_s_at Cluster Incl. U68487:Human 5-hydroxytryptamine7 receptor isoform b nmR; 35536_at Cluster Incl. AB011176:Homo sapiens mRNA for KIAA0604 protein, complete; 32923_r_at Cluster Incl. M58378:Human synapsin I (SYN1) gene /cds=(0,2117) /gb=M; 34906_g_at Cluster Incl. AA977136:oq24f02.s1 Homo sapiens cDNA, 3 end /clone=IM; 35899_at Cluster Incl. AF109401:Homo sapiens neurotrophic factor artemin precurs; 41867_at Cluster Incl. AF055009:Homo sapiens clone 24747 mRNA sequence /cds=UNKN; 31861_at Cluster Incl. L14754:Human DNA-binding protein (SMBP2) mRNA, complete c; 32660_at Cluster Incl. AB002340:Human mRNA for KIAA0342 gene, complete cds /cds=; 36011_at Cluster Incl. AF035531:Homo sapiens syntaxin 10 mRNA, complete cds /cds; 36482_s_at Cluster Incl. Y15724:Homo sapiens SERCA3 gene, exons 1-7 (and joined; 36907_at Cluster Incl. M88468:Homo sapiens mevalonate kinase mRNA, complete cds; 37939_at Cluster Incl. AL022318:bK150C2.3 (PUTATIVE novel protein similar to APO; 39383_at Cluster Incl. AB007882:Homo sapiens KIAA0422 mRNA, partial cds /cds=(0,; 36129_at Cluster Incl. AB007857:Homo sapiens KIAA0397 mRNA, complete cds /cds=(5; 38088_r_at Cluster Incl. M80563:Human CAPL protein mRNA, complete cds /cds=(135,; 39490_f_at Cluster Incl. W26381:29b11 Homo sapiens cDNA /gb=W26381 /gi=1307098 /; 39908_at Cluster Incl. AF069735:Homo sapiens PCAF associated factor 65 alpha mRN; 40595_at Cluster Incl. AI345337:tb81g11.x1 Homo sapiens cDNA, 3 end /clone=IMAG; 1469_at U12779 /FEATURE= /DEFINITION=HSU12779 Human MAP kinase activated protein; 1392_at L16862 /FEATURE= /DEFINITION=HHUMPROCRKI Homo sapiens G protein-coupled r; 1270_at M64788 /FEATURE= /DEFINITION=HUMRAP1GAP Human GTPase activating protein; 1032_at U11872 /FEATURE= /DEFINITION=HSU11872 Human interleukin-8 receptor type; 882_at M37435 /FEATURE= /DEFINITION=HUMCSDF1 Human macrophage-specific colony-st; 771_s_at D00749 /FEATURE=cds /DEFINITION=HUMCD7G3 Human T cell surface antigen C; 534_s_at U20391 /FEATURE=mRNA#1 /DEFINITION=HSU20391 Human folate receptor (FOLR.

Metagene 326; 33250_at Cluster Incl. AL031228:dJ1033B10.2 (WD40 protein BING4 (similar to S. c; 34679_at Cluster Incl. X02596:Human mRNA for bcr (breakpoint cluster region) gen; 32818_at Cluster Incl. X78565:H. sapiens mRNA for tenascin-C, 7560 bp /cds=(313,69; 36195_at Cluster Incl. U07681:Human NAD(H)-specific isocitrate dehydrogenase alp; 38797_at Cluster Incl. D31887:Human mRNA for KIAA0062 gene, partial cds /cds=(0,; 40198_at Cluster Incl. L06132:Human voltage-dependent anion channel isoform 1 (V; 41485_at Cluster Incl. X02152:Human mRNA for lactate dehydrogenase-A (LDH-A, EC; 2031_s_at U03106 /FEATURE= /DEFINITION=HSU03106 Human wild-type p53 activated fr; 1333_f_at X02596 /FEATURE=cds /DEFINITION=HSBCRR Human mRNA for bcr (breakpoint; 1199_at D13748 /FEATURE= /DEFINITION=HUM4AI Human mRNA for eukaryotic initiation; 867_s_at U12471 /FEATURE=cds#2 /DEFINITION=HSU12471 Human thrombospondin-1 gene,; 537_f_at U07000 /FEATURE=cds#3 /DEFINITION=HSU07000 Human breakpoint cluster reg; 430_at X00737 /FEATURE=cds /DEFINITION=HSPNP Human mRNA for purine nucleoside ph.

Metagene 327; 37430_at Cluster Incl. U78294:Homo sapiens 15S-lipoxygenase mRNA, complete cds /; 38597_f_at Cluster Incl. D50402:Human mRNA for NRAMP1, complete cds /cds=(175,18; 40730_at Cluster Incl. U48263:Human pre-pro-orphanin FQ (OFQ) mRNA, complete cds; 32103_at Cluster Incl. M20786:Human alpha-2-plasmin inhibitor gene /cds=(4,1479); 40506_s_at Cluster Incl. U75686:Homo sapiens polyadenylate binding protein mRNA,; 41778_at Cluster Incl. U53347:Human neutral amino acid transporter B mRNA, compl; 34378_at Cluster Incl. X97324:H. sapiens mRNA for adipophilin /cds=(0,1313) /gb=X; 34892_at Cluster Incl. AF016266:Homo sapiens TRAIL receptor 2 mRNA, complete cds; 36155_at Cluster Incl. D87465:Human mRNA for KIAA0275 gene, complete cds /cds=(3; 1491_at M31166 /FEATURE=mRNA /DEFINITION=HUMTSG14A Human tumor necrosis factor-i.

Metagene 328; 34494_at Cluster Incl. AJ003125:Homo sapiens mRNA for procollagen I—N proteinase; 41440_at Cluster Incl. D82061:Homo sapiens mRNA for a member of the short-chain; 33235_at Cluster Incl. AB023155:Homo sapiens mRNA for KIAA0938 protein, complete; 36147_at Cluster Incl. X74104:H. sapiens mRNA for TRAP beta subunit /cds=(50,601); 40257_at Cluster Incl. AI400011:tg85a05.x1 Homo sapiens cDNA, 3 end /clone=IMAG; 32533_s_at Cluster Incl. AF054825:Homo sapiens VAMP5 mRNA, complete cds /cds=(57.

Metagene 329; 31675_s_at Cluster Incl. AF019083:Homo sapiens phosphatase and tensin homolog 2; 31950_at Cluster Incl. Y00345:Human mRNA for polyA binding protein /cds=(502,240; 37819_at Cluster Incl. AF007130:Homo sapiens clone 23750 unknown mRNA, partial c; 38924_s_at Cluster Incl. AF001628:Homo sapiens interactor protein AblBP4 (AblBP4; 39231_at Cluster Incl. AF006513:Homo sapiens CHD1 mRNA, complete cds /cds=(163,5; 39317_at Cluster Incl. D86324:Homo sapiens mRNA for CMP-N-acetylneuraminic acid; 39964_at Cluster Incl. AJ007590:Homo sapiens mRNA for XRP2 protein /cds=(172,122; 41620_at Cluster Incl. AB018259:Homo sapiens mRNA for KIAA0716 protein, complete; 41621_i_at Cluster Incl. AA868898:ak55b08.s1 Homo sapiens cDNA, 3 end /clone=IM; 41651_at Cluster Incl. AB028956:Homo sapiens mRNA for KIAA1033 protein, partial; 32059_at Cluster Incl. U79282:Human clone 23801 mRNA sequence /cds=UNKNOWN /gb=U; 36060_at Cluster Incl. U51920:Human signal recognition particle (SRP54) mRNA, co; 36502_at Cluster Incl. AB020641:Homo sapiens mRNA for KIAA0834 protein, complete; 36550_at Cluster Incl. AL049538:Human DNA sequence from clone 117516 on chromoso; 36898_r_at Cluster Incl. X74331:H. sapiens mRNA for DNA primase (subunit p58) /cd; 37255_at Cluster Incl. U36601:Homo sapiens heparan N-deacetylase/N-sulfotransfer; 37936_at Cluster Incl. AI184802:qd24g04.x1 Homo sapiens cDNA, 3 end /clone=IMAG; 37971_at Cluster Incl. AL050089:Homo sapiens mRNA; cDNA DKFZp586E0518 (from clon; 38291_at Cluster Incl. J00123:Human enkephalin gene /cds=(0,803) /gb=J00123 /gi=; 38670_at Cluster Incl. AL031685:dJ963K23.2 (novel protein) /cds=(2,688) /gb-AL03; 38972_at Cluster Incl. AF052169:Homo sapiens clone 24775 mRNA sequence /cds=UNKN; 39032_at Cluster Incl. AJ222700:Homo sapiens mRNA for TSC-22 protein /cds=(191,6; 39396_at Cluster Incl. AF081281:Homo sapiens lysophospholipase (LPL1) mRNA, comp; 39434_at Cluster Incl. AB011164:Homo sapiens mRNA for KIAA0592 protein, partial; 39744_at Cluster Incl. AF000982:Homo sapiens dead box, X isoform (DBX) mRNA, alt; 40083_at Cluster Incl. AB014525:Homo sapiens mRNA for KIAA0625 protein, partial; 40088_at Cluster Incl. X84373:H. sapiens mRNA for nuclear factor RIP140 /cds=(287; 40453_s_at Cluster Incl. U30826:Human splicing factor SRp40-1 (SRp40) mRNA, comp; 40767_at Cluster Incl. M59499:Human lipoprotein-associated coagulation inhibitor; 40803_at Cluster Incl. AL050161:Homo sapiens mRNA; cDNA DKFZp586B0222 (from clon; 40878_f_at Cluster Incl. AF041081:Homo sapiens D15F37 pseudogene, S4 allele, mRN; 41175_at Cluster Incl. L20298:Homo sapiens transcription factor (CBFB) mRNA, 3; 41767_r_at Cluster Incl. AB020662:Homo sapiens mRNA for KIAA0855 protein, partia; 32215_i_at Cluster Incl. AB020685:Homo sapiens mRNA for KIAA0878 protein, comple; 33893_r_at Cluster Incl. AB007939:Homo sapiens mRNA for KIAA0470 protein, comple; 34386_at Cluster Incl. AF072250:Homo sapiens methyl-CpG binding protein MBD4 (MB; 34393_r_at Cluster Incl. AL050268:Homo sapiens mRNA; cDNA DKFZp564B163 (from clo; 34876_at Cluster Incl. U65090:Human carboxypeptidase D mRNA, complete cds /cds=(; 35803_at Cluster Incl. S82240:RhoE=26 kda GTPase homolog [human, HeLa cell line,; 35811_at Cluster Incl. AF037204:Homo sapiens RING zinc finger protein (RZF) mRNA; 36572_r_at Cluster Incl. D31885:Human mRNA for KIAA0069 gene, partial cds /cds=(; 37294_at Cluster Incl. X61123:Human BTG1 mRNA /cds=(308,823) /gb=X61123 /gi=2950; 37651_at Cluster Incl. D31888:Human mRNA for KIAA0071 gene, partial cds /cds=(0,; 37685_at Cluster Incl. U45976:Human clathrin assembly protein lymphoid myeloid 1; 38041_at Cluster Incl. U41514:Human UDP-GalNAc-polypeptide N-acetylgalactosaminy; 38084_at Cluster Incl. AA648295:ns20e08.s1 Homo sapiens cDNA, 3 end /clone=IMAG; 38110_at Cluster Incl. AF000652:Homo sapiens syntenin (sycl) mRNA, complete cds; 38392_at Cluster Incl. AF006088:Homo sapiens Arp2/3 protein complex subunit p16-; 38402_at Cluster Incl. U36336:Human lysosome-associated membrane protein-2b (LAM; 38764_at Cluster Incl. AF007142:Homo sapiens clone 23938 mRNA sequence lcds=UNKN; 38820_at Cluster Incl. AF051894:Homo sapiens 15 kDa selenoprotein mRNA, complete; 39506_at Cluster Incl. AA933984:on95f04.s1 Homo sapiens cDNA, 3 end /clone=IMAG; 32504_at Cluster Incl. AW024812:wu69c05.x1 Homo sapiens cDNA, 3 end /clone=IMAG; 1463_at M93425 /FEATURE= /DEFINITION=HUMPTPPEST Human protein tyrosine phosphata; 1377_at M58603 /FEATURE= /DEFINITION=HUMNFKB Human nuclear factor kappa-B DNA bi; 1038_s_at U19247 /FEATURE=mRNA /DEFINITION=HSINFGRA7 Homo sapiens interferon-gam; 763_at AB001106 /FEATURE= /DEFINITION=AB001106 Homo sapiens mRNA for glia matura; 377_g_at AB000220 /FEATURE= /DEFINITION=AB000220 Homo sapiens mRNA for semaphori.

Metagene 330; 31733_at Cluster Incl. Y07683:H. sapiens mRNA for P2X3 purinoceptor /cds=(165,135; 38276_at Cluster Incl. U91616:Human I kappa B epsilon (IkBe) mRNA, complete cds; 32514_s_at Cluster Incl. AF032906:Homo sapiens cathepsin Z precursor (CTSZ) mRNA; 652_g_at L07493 /FEATURE= /DEFINITION=HUM14RPA Homo sapiens replication protein.

Metagene 331; 31783_at Cluster Incl. U52112:renin-binding protein /cds=(191,1444) /gb=U52112 /; 33641_g_at Cluster Incl. Y14768:Homo sapiens DNA, cosmid clones TN62 and TN82 /c; 35132_at Cluster Incl. X98411:H. sapiens mRNA for myosin-IE /cds=(0,2348) /gb=X98; 36372_at Cluster Incl. U51333:Human hexokinase III (HK3) mRNA, complete cds /cds; 34435_at Cluster Incl. AB008775:Homo sapiens AQP9 mRNA for aquaporin 9, complete; 35926_s_at Cluster Incl. AF004230:Homo sapiens monocyte/macrophage Ig-related re; 36781_at Cluster Incl. X01683:Human mRNA for alpha 1-antitrypsin /cds=(38,1294); 37100_at Cluster Incl. AJ008112:Homo sapiens mRNA for C17orf1 protein /cds=(39,1; 38129_at Cluster Incl. L13943:Human glycerol kinase (GK) mRNA exons 1-4, complet; 38533_s_at Cluster Incl. J03925:Human Mac-1 gene encoding complement receptor ty; 39649_at Cluster Incl. X78817:H. sapiens partial C1 mRNA /cds=(42,2882) /gb=X7881; 39950_at Cluster Incl. Y08136:H. sapiens mRNA for ASM-like phosphodiesterase 3a /; 40698_at Cluster Incl. X96719:H. sapiens mRNA for AICL (activation-induced C-type; 40712_at Cluster Incl. D26579:Homo sapiens mRNA for transmembrane protein, compl; 40742_at Cluster Incl. M16591:Human hemopoietic cell protein-tyrosine kinase (HC; 32112_s_at Cluster Incl. AI800499:tc11f11.x1 Homo sapiens cDNA, 3 end /clone=IM; 32736_at Cluster Incl. W68830:zd37g06.r1 Homo sapiens cDNA, 5 end /clone=IMAGE-; 33748_at Cluster Incl. D86976:Human mRNA for KIAA0223 gene, partial cds /cds=(0,; 33802_at Cluster Incl. Z82244:bK286B10.2 (Heme Oxygenase 1 (HO-1, EC 1.14.99.3)); 34210_at Cluster Incl. N90866:zb11b10.s1 Homo sapiens cDNA, 3 end /clone=IMAGE-; 36028_at Cluster Incl. U45285:Human specific 116-kDa vacuolar proton pump subuni; 36833_at Cluster Incl. U78027:Homo sapiens Brutons tyrosine kinase (BTK), alpha-; 37220_at Cluster Incl. M63835:Human IgG Fc receptor I gene /cds=(155,1279) /gb=M; 37536_at Cluster Incl. Z11697:Homo sapiens mRNA for HB15 /cds=(10,627) /gb=Z1169; 37967_at Cluster Incl. AF000424:Homo sapiens LST1 mRNA, cLST1/C splice variant,; 38666_at Cluster Incl. M85169:Human homologue of yeast sec7 mRNA, complete cds /; 38976_at Cluster Incl. D44497:Human mRNA for actin binding protein p57, complete; 39799_at Cluster Incl. M94856:Human fatty acid binding protein homologue (PA-FAB; 40518_at Cluster Incl. Y00062:Human mRNA for T200 leukocyte common antigen (CD45; 41169_at Cluster Incl. X74039:H. sapiens mRNA for urokinase plasminogen activator; 32824_at Cluster Incl. AF039704:Homo sapiens lysosomal pepstatin insensitive pro; 35261_at Cluster Incl. W07033:za93f08.r1 Homo sapiens cDNA, 5 end /clone-IMAGE-; 36103_at Cluster Incl. D90144:Homo sapiens gene for LD78 alpha precursor, comple; 36674_at Cluster Incl. J04130:Human activation (Act-2) mRNA, complete cds /cds=(; 37011_at Cluster Incl. U49392:Human allograft inflammatory factor-1 (AIF-1) mRNA; 37023_at Cluster Incl. J02923:Human 65-kilodalton phosphoprotein (p65) mRNA, com; 37688_f_at Cluster Incl. M31932:Human IgG low affinity Fc fragment receptor (FcR; 38038_at Cluster Incl. U21128:Human lumican mRNA, complete cds /cds=(84,1100) /g; 41814_at Cluster Incl. M29877:Human alpha-L-fucosidase, complete cds /cds=UNKNOW; 32570_at Cluster Incl. L76465:Homo sapiens NAD+-dependent 15 hydroxyprostaglandi; 33106_at Cluster Incl. U22662:Human nuclear orphan receptor LXR-alpha mRNA, comp; 2045_s_at M116592 /FEATURE=mRNA /DEFINITION=HUMHCKB Human hemopoietic cell protei; 2002_s_at U27467 /FEATURE= /DEFINITION=HSU27467 Human Bcl-2 related (Bfl-1) mRNA; 1665_s_at Endothelial Cell Growth Factor 1; 1506_at D11086 /FEATURE= /DEFINITION=HUMIL2RG Human mRNA for interleukin 2 recep; 1402_at M16038 /FEATURE= /DEFINITION=HUMLYN Human lyn mRNA encoding a tyrosine k; 1127_at L07597 /FEATURE= /DEFINITION=HUMS6KINA Homo sapiens ribosomal protein S6; 341_at D10202 /FEATURE= /DEFINITION=HUMPAFRE Homo sapiens mRNA for platelet-acti; 210_at M95678 /FEATURE= /DEFINITION=HUMPLCB2A Homo sapiens phospholipase C-beta-.

Metagene 332; 33670_at Cluster Incl. X74439:H. sapiens mRNA for estrogen receptor gene 5flankin; 32281_at Cluster Incl. AF070549:Homo sapiens clone 24475 mRNA sequence /cds=UNKN; 35932_at Cluster Incl. AF081507:untitled /cds=(70,1170) /gb=AF081507 /gi=3450860; 37157_at Cluster Incl. X56667:Human mRNA for calretinin /cds=(43,858) /gb=X56667; 33336_at Cluster Incl. M27819:Human anion exchange protein 1 (AE1, band 3) mRNA,; 39006_r_at Cluster Incl. AB018257:Homo sapiens mRNA for KIAA0714 protein, partia; 34308_at Cluster Incl. U90551:Human histone 2A-like protein (H2A/I) mRNA, comple; 35735_at Cluster Incl. M55542:Human guanylate binding protein isoform I (GBP-2); 40974_at Cluster Incl. U63541:Human mRNA expressed in HC/HCC livers and MolT-4 p.

Metagene 333; 31661_at Cluster Incl. AJ243936:Homo sapiens mRNA for G16 protein (G16 gene loca; 33071_at Cluster Incl. Z98744:histone H2B /cds=(5,382) /gb=Z98744 /gi=3080⁴57 /u; 36398_at Cluster Incl. W28729:50h2 Homo sapiens cDNA /gb=W28729 /gi=1308677 /ug=; 34457_at Cluster Incl. U76010:Human putative zinc transporter ZnT-3 (ZnT-3) mRNA; 34527_r_at Cluster Incl. AF052108:Homo sapiens clone 23687 mRNA sequence /cds=UN; 35915_at Cluster Incl. X82540:H. sapiens mRNA for activin beta-C chain /cds=(127,; 36242_at Cluster Incl. M21539:Human small proline rich protein (sprII) mRNA, clo; 36702_at Cluster Incl. AJ010277:Homo sapiens mRNA for TBX19 protein /cds=(51,139; 39306_at Cluster Incl. AF052514:Homo sapiens thymus specific serine peptidase mR; 41101_at Cluster Incl. D87464:Human mRNA for KIAA0274 gene, complete cds /cds=(1; 32717_at Cluster Incl. AF029729:Homo sapiens neuralized mRNA, complete cds /cds=; 33757_f_at Cluster Incl. M69245:Human pregnancy-specific beta-1 glycoprotein (PS; 41227_at Cluster Incl. AL022162:Homo sapiens DNA sequence from PAC 454M7 on chro; 38008_at Cluster Incl. M98528:Homo sapiens neuron-specific protein gene, last ex; 38024_at Cluster Incl. AB020678:Homo sapiens mRNA for KIAA0871 protein, complete; 38080_at Cluster Incl. AI525665:PT1.3_{—}04_D06.r Homo sapiens cDNA, 5 end /clone_{—}; 41351_at Cluster Incl. AA885106:am31h01.s1 Homo sapiens cDNA, 3 end /clone=IMAG; 41799_at Cluster Incl. W28595:48h8 Homo sapiens cDNA /gb=W28595 /gi=1308543 /ug=; 2027_at M87068 /FEATURE= /DEFINITION=HUMCAN H. sapiens CaN19 mRNA sequence; 1792_g_at M68520 /FEATURE= /DEFINITION=HUMCDC2A Human cdc2-related protein kinas; 1014_at U60325 /FEATURE= /DEFINITION=HSU60325 Human DNA polymerase gamma mRNA, n; 866_at U12471 /FEATURE=cds#1 /DEFINITION=HSU12471 Human thrombospondin-1 gene, p; 732_f_at Mucin 3, Intestinal; 207_at M86752 /FEATURE= /DEFINITION=HUMIEF Human transformation-sensitive protei.

Metagene 334; 32963_s_at Cluster Incl. W27549:32d11 Homo sapiens cDNA /gb=W27549 /gi=1307353 /; 37873_g_at Cluster Incl. AF072468:Homo sapiens (JH8) mRNA, partial cds /cds=(0,1; 39566_at Cluster Incl. X70297:H. sapiens mRNA for neuronal nicotinic acetylcholin; 31863_at Cluster Incl. D80001:Human mRNA for KIAA0179 gene, partial cds /cds=(0,; 34661_at Cluster Incl. AB002348:Human mRNA for KLAA0350 gene, partial cds /cds=(; 37914_at Cluster Incl. AB002303:Human mRNA for KIAA0305 gene, complete cds /cds=; 38687_at Cluster Incl. AL050051:Homo sapiens mRNA; cDNA DKFZp566D193 (from clone; 34337_s_at Cluster Incl. AJ010014:Homo sapiens mRNA for M96A protein /cds=(243,2; 34353_at Cluster Incl. AB014548:Homo sapiens mRNA for KIAA0648 protein, partial; 38782_at Cluster Incl. M95809:Human basic transcription factor 62 kD subunit (BTF.

Metagene 335; 36354_at Cluster Incl. AL049689:Novel human mRNA from chromosome 1, similar to T; 34701_at Cluster Incl. U83192:Homo sapiens post-synaptic density protein 95 (PSD; 36499_at Cluster Incl. D87469:Human mRNA for KIAA0279 gene, partial cds /cds=(0,; 41357_at Cluster Incl. W27997:43e3 Homo sapiens cDNA /gb=W27997 /gi=1308152 /ug=.

Metagene 336; 35665_at Cluster Incl. Z46973:H. sapiens mRNA for phosphatidylinositol 3-kinase /; 33395_at Cluster Incl. AL050028:Homo sapiens mRNA; cDNA DKFZp566C0424 (from clon.

Metagene 337; 31521_f_at Cluster Incl. X60484:H. sapiens H4 /e gene for H4 histone /cds=(0,311); 31948_at Cluster Incl. X79563:H. sapiens 8.2 kDa differentiation factor mRNA /cds=; 39953_i_at Cluster Incl. AB014528:Homo sapiens mRNA for KLAA0628 protein, comple; 32062_at Cluster Incl. D25216:Human mRNA for KIAA0014 gene, complete cds /cds=(1; 33797_at Cluster Incl. X98494:H. sapiens mRNA for M phase phosphoprotein 10 /cds=; 35159_at Cluster Incl. U61232:Human tubulin-folding cofactor E mRNA, complete cd; 35212_at Cluster Incl. AF064801:Homo sapiens multiple membrane spanning receptor; 36068_at Cluster Incl. AF002210:Homo sapiens copper chaperone for superoxide dis; 36847_r_at Cluster Incl. AA121509:zk88c10.s1 Homo sapiens cDNA, 3 end /clone=IM; 36928_at Cluster Incl. X70394:H. sapiens OZF mRNA /cds=(856,1734) /gb=X70394 /gi=; 37964_at Cluster Incl. W25793:13e7 Homo sapiens cDNA /gb=W25793 /gi=1305934 /ug=; 40112_at Cluster Incl. AA522698:ni39d03.s1 Homo sapiens cDNA, 3 end /clone=IMAG; 40118_at Cluster Incl. X07290:Human HF.12 gene mRNA /cds=(0,589) /gb=X07290 /gi=; 40872_at Cluster Incl. T57872:yb19b12.s1 Homo sapiens cDNA, 3 end /clone=IMAGE-; 32194_at Cluster Incl. M37197:Human CCAAT-box-binding factor (CBF) mRNA, complet; 32236_at Cluster Incl. AF032456:Homo sapiens ubiquitin conjugating enzyme G2 (UB; 33844_at Cluster Incl. AA160724:zo72h04.r1 Homo sapiens cDNA, 5 end /clone=IMAG; 33861_at Cluster Incl. AI123426:qa49c09.x1 Homo sapiens cDNA, 3 end /clone=IMAG; 34801_at Cluster Incl. AB014610:Homo sapiens mRNA for KIAA0710 protein, complete; 34817_s_at Cluster Incl. U70671:Human ataxin-2 related protein mRNA, partial cds; 35267_g_at Cluster Incl. AL049288:Homo sapiens mRNA; cDNA DKFZp564M053 (from clo; 35739_at Cluster Incl. AB002369:Human mRNA for KIAA0371 gene, complete cds /cds=; 35776_at Cluster Incl. AF064243:Homo sapiens intersectin short form mRNA, comple; 35783_at Cluster Incl. H93123:yv05g07.s1 Homo sapiens cDNA, 3 end /clone=IMAGE-; 38416_at Cluster Incl. L27706:Human chaperonin protein (Tcp20) gene complete cds; 38774_at Cluster Incl. U77942:Human syntaxin 7 mRNA, complete cds /cds=(79,864); 38778_at Cluster Incl. AB028969:Homo sapiens mRNA for KIAA1046 protein, complete; 39921_at Cluster Incl. AI526089:DU3.2-7.H07.r Homo sapiens cDNA, 5 end /clone-e; 40564_at Cluster Incl. N42007:yw69e06.r1 Homo sapiens cDNA, 5 end /clone=IMAGE-; 40569_at Cluster Incl. M58297:Human zinc finger protein 42 (MZF-1) mRNA, complet; 41329_at Cluster Incl. AI458463:tj99b02.x1 Homo sapiens cDNA, 3 end /clone=IMAG; 41512_at Cluster Incl. AL042733:DKFZp434B2222_s1 Homo sapiens cDNA, 3 end /clon; 41832_s_at Cluster Incl. AI535828:jun1.P1.H10 Homo sapiens cDNA, 3 end /clone-e; 1873_at D21089 /FEATURE= /DEFINITION=HUMXPCR Human mRNA for XP-C repair compleme; 947_at D55716 /FEATURE= /DEFINITION=HUMPICDC47 Human mRNA for P1cdc47, complete; 782_at U93867 /FEATURE= /DEFINITION=HSU93867 Human RNA polymerase III subunit (R; 250_at L41067 /FEATURE= /DEFINITION=HUMHFAT4A Homo sapiens NF-AT4c mRNA, complet.

Metagene 338; 32387_at Cluster Incl. AB017494:Homo sapiens mRNA for LCAT-like lysophospholipas; 41226_at Cluster Incl. L05147:Human dual specificity phosphatase tyrosine/serine; 41494_at Cluster Incl. X99802:H. sapiens mRNA for ZYG homologue /cds=(38,2338) /g; 1834_at D38449 /FEATURE= /DEFINITION=HUMGPCRAA Human mRNA for G protein-coupled; 1750_at AD000092 /FEATURE=cds#5 /DEFINITION=CH19HHR23 Homo sapiens DNA from chro; 1753_s_at AD000092 /FEATURE=cds#7 /DEFINITION=CH19HHR23 Homo sapiens DNA from ch; 1558_g_at U24152 /FEATURE= /DEFINITION=HSU24152 Human p21-activated protein kina.

Metagene 339; 31621_s_at Cluster Incl. M36860:Human elastin mRNA, complete cds /cds=(49,2241); 40667_at Cluster Incl. X60992:H. sapiens CD6 mRNA for T cell glycoprotein CD6 /cd; 32189_g_at Cluster Incl. M96980:Homo sapiens myelin transcription factor 1 (MTF1; 40600_at Cluster Incl. AW024467:wu76g06.x1 Homo sapiens cDNA, 3 end /clone=IMAG; 2067_f_at L22475 /FEATURE= /DEFINITION=HUMBAXG Human Bax gamma mRNA, complete cd; 1517_at J02906 /FEATURE=mRNA /DEFINITION=HUMCYPIIF Human cytochrome P450IIF1 pro; 671_at J03040 /FEATURE= /DEFINITION=HUMSPARC Human SPARC/osteonectin mRNA, compl.

Metagene 340; 36391_at Cluster Incl. AF048730:Homo sapiens cyclin T1 mRNA, complete cds /cds=(; 34032_at Cluster Incl. AL109707:Homo sapiens mRNA full length insert cDNA clone; 34936_at Cluster Incl. AB012130:Homo sapiens SBC2 mRNA for sodium bicarbonate co; 36225_s_at Cluster Incl. W27611:35b9 Homo sapiens cDNA /gb=W27611 /gi=1307559 /u; 38962_at Cluster Incl. AB002296:Human mRNA for KIAA0298 gene, complete cds /cds=; 39286_at Cluster Incl. D64109:Homo sapiens mRNA for tob family, complete cds /cd; 39634_at Cluster Incl. AB017168:Homo sapiens mRNA for Slit-2 protein, complete c; 41465_at Cluster Incl. AJ236885:Homo sapiens mRNA for ZBP-89 protein /cds=(391,2; 34211_at Cluster Incl. AL079697:DKFZp434E1930_r1 Homo sapiens cDNA, 5 end /clon; 34676_at Cluster Incl. AB029022:Homo sapiens mRNA for KIAA1099 protein, complete; 37947_at Cluster Incl. D26362:Human mRNA for KIAA0043 gene, complete cds /cds=(1; 39343_at Cluster Incl. AW026656:wv15c06.x1 Homo sapiens cDNA, 3 end /clone=IMAG; 33349_at Cluster Incl. AL049378:Homo sapiens mRNA; cDNA DKFZp586I1518 (from clon; 33862_at Cluster Incl. AF017786:Homo sapiens phosphatidic acid phosphohydrolase; 39450_s_at Cluster Incl. AF050145:Homo sapiens iduronate-2-sulfatase (IDS) mRNA,; 39551_at Cluster Incl. N98667:yy66d05.r1 Homo sapiens cDNA, 5 end /clone=IMAGE-; 40949_at Cluster Incl. AF035812:Homo sapiens dynein light intermediate chain 2 (; 1602_at L33881 /FEATURE= /DEFINITION=HUMPKCI Human protein kinase C iota isoform; 1319_at X74764 /FEATURE=cds /DEFINITION=HSRPTK H. sapiens mRNA for receptor prote; 932_i_at L11672 /FEATURE= /DEFINITION=HUMKRUPZN Human Kruppel related zinc finge; 462_at U70862 /FEATURE= /DEFINITION=HSU70862 Human nuclear factor I B3 mRNA, com; 387_at X80230 /FEATURE=mRNA /DEFINITION=HSSTPKC2K H. sapiens mRNA (clone C-2k) mR.

Metagene 341; 36421_at Cluster Incl. AF038194:Homo sapiens clone 23821 mRNA sequence /cds=UNKN; 36010_at Cluster Incl. U10492:Human Mox1 protein (MOX1) mRNA, complete cds /cds=; 36065_at Cluster Incl. AF052389:Homo sapiens LIM domain binding protein (LDB1) m; 37550_at Cluster Incl. M14113:Human coagulation factor VIII-C mRNA, complete cds; 37919_at Cluster Incl. AF056732:untitled /cds=(83,2014) /gb=AF056732 /gi=3676521; 38994_at Cluster Incl. AF037989:Homo sapiens STAT-induced STAT inhibitor-2 mRNA,; 39048_at Cluster Incl. U95299:Human Notch4 (hNotch4) mRNA, complete cds /cds=(90; 39398_s_at Cluster Incl. AB023205:Homo sapiens mRNA for KIAA0988 protein, comple; 40458_at Cluster Incl. U43185:Human signal transducer and activator of transcrip; 37709_at Cluster Incl. M86934:Human GS1 (protein of unknown function) mRNA, comp; 37718_at Cluster Incl. D43636:Human mRNA for KIAA0096 gene, partial cds /cds=(0,; 525_g_at U13695 /FEATURE=cds /DEFINITION=HSU13695 Human homolog of yeast mutL (h.

Metagene 342; 35235_at Cluster Incl. AA099265:zk84f07.r1 Homo sapiens cDNA, 5 end /clone=IMAG; 38986_at Cluster Incl. Z49835:H. sapiens mRNA for protein disulfide isomerase /cd; 38987_at Cluster Incl. AF052183:Homo sapiens clone 24804 mRNA sequence /cds=UNKN; 33421_s_at Cluster Incl. AB016247:Homo sapiens mRNA for sterol-C5-desaturase, co; 37654_at Cluster Incl. D31764:Human mRNA for KIAA0064 gene, complete cds /cds=(2.

Metagene 343; 38565_at Cluster Incl. D88667:Homo sapiens mRNA for cerebroside sulfotransferase; 32657_at Cluster Incl. D25278:Human mRNA for KIAA0036 gene, complete cds /cds=(1.

Metagene 344; 33631_at Cluster Incl. AF023612:Homo sapiens Dim1p homolog mRNA, complete cds /c; 37114_at Cluster Incl. L32832:Homo sapiens zinc finger homeodomain protein (ATBF; 38188_s_at Cluster Incl. L28821:Homo sapiens alpha mannosidase II isozyme mRNA,; 39587_at Cluster Incl. AJ005890:Homo sapiens mRNA for JM1 protein, complete CDS; 40016_g_at Cluster Incl. AB002301:Human mRNA for KIAA0303 gene, partial cds /cds; 41010_at Cluster Incl. Y17829:Homo sapiens mRNA for Homer-related protein Syn47; 34178_at Cluster Incl. AI884738:wl84b02.x1 Homo sapiens cDNA, 3 end /clone=IMAG; 34264_at Cluster Incl. AB026894:Homo sapiens mRNA for NESCA, complete cds /cds=(; 36519_at Cluster Incl. M13194:Human excision repair protein (ERCC1) mRNA, comple; 36562_at Cluster Incl. AB007887:Homo sapiens KIAA0427 mRNA, complete cds /cds=(2; 38257_at Cluster Incl. AF038406:Homo sapiens NADH dehydrogenase-ubiquinone Fe—S; 40426_at Cluster Incl. X89985:H. sapiens mRNA for BCL7B protein /cds=(37,645) /gb; 40497_at Cluster Incl. AF040707:Homo sapiens candidate tumor suppressor gene 21; 40825_at Cluster Incl. AB025186:Homo sapiens mRNA for EB3 protein, complete cds; 33437_at Cluster Incl. AJ005892:Homo sapiens mRNA for JM23 protein, complete cod; 33901_at Cluster Incl. U81375:Human placental equilibrative nucleoside transport; 35308_at Cluster Incl. D83200:Homo sapiens mRNA expressed in placenta /cds=UNKNO; 36679_at Cluster Incl. X06272:Human mRNA for docking protein (signal recognition; 37768_at Cluster Incl. M74905:Human 3-alkyladenine DNA glycosylase (HAAG) mRNA,; 38021_at Cluster Incl. U53204:Human plectin (PLEC1) mRNA, complete cds /cds=(51,; 38094_at Cluster Incl. M65028:Human hnRNP type A/B protein mRNA, complete cds /c; 38730_at Cluster Incl. AB020671:Homo sapiens mRNA for KIAA0864 protein, partial; 39088_at Cluster Incl. Y18007:Homo sapiens mRNA for putative seven transmembrane; 32574_at Cluster Incl. X59960:H. sapiens mRNA for sphingomyelinase /cds=(122,2005; 1241_at U14603 /FEATURE= /DEFINITION=HSU14603 Human protein-tyrosine phosphatase.

Metagene 345; 35584_s_at Cluster Incl. AJ224874:Homo sapiens mRNA for L-type calcium channel a; 35861_at Cluster Incl. AA018440:ze50a07.s1 Homo sapiens cDNA, 3 end /clone=IMAG; 36286_at Cluster Incl. AF070628:Homo sapiens clone 24803 mRNA sequence /cds=UNKN; 37228_at Cluster Incl. U01038:Human pLK mRNA, complete cds /cds=(63,1874) /gb=U0; 39417_at Cluster Incl. AB028951:Homo sapiens mRNA for KIAA1028 protein, partial; 40443_at Cluster Incl. AF062341:Homo sapiens p120 catenin isoform 1ABC (CTNND1); 40232_at Cluster Incl. U75370:Human mitochondrial RNA polymerase mRNA, nuclear g.

Metagene 346; 31755_at Cluster Incl. AI094859:qa09a09.x1 Homo sapiens cDNA, 3 end /clone=IMAG; 34602_at Cluster Incl. D63160:Homo sapiens DNA for lectin P35 /cds=(10,951) /gb=; 35544_at Cluster Incl. Y16280:Homo sapiens mRNA for G protein-coupled receptor E; 34905_at Cluster Incl. AA977136:oq24f02.s1 Homo sapiens cDNA, 3 end /clone=IMAG; 36803_at Cluster Incl. L77561:Homo sapiens DGS-D mRNA, 3 end /cds=UNKNOWN /gb=L; 37082_at Cluster Incl. AB007886:Homo sapiens KIAA0426 mRNA, complete cds /cds=(1; 39650_s_at Cluster Incl. AB007895:Homo sapiens KIAA0435 mRNA, complete cds /cds=; 41664_at Cluster Incl. AF026030:Homo sapiens putative mitochondrial inner membra; 41869_at Cluster Incl. U78310:Homo sapiens pescadillo mRNA, complete cds /cds=(5; 31839_at Cluster Incl. AC004475:Homo sapiens chromosome 19, cosmid F23858 /cds=(; 32095_at Cluster Incl. AB018267:Homo sapiens mRNA for KIAA0724 protein, complete; 32122_at Cluster Incl. L31573:Human sulfite oxidase mRNA, complete cds /cds=(903; 32681_at Cluster Incl. S68616:Na+/H+ exchanger NHE-1 isoform [human, heart, mRNA; 33225_at Cluster Incl. AI928387:wo96f10.x1 Homo sapiens cDNA, 3 end /clone=IMAG; 33307_at Cluster Incl. AL022316:Human DNA sequence from clone 126B4 on chromosom; 35156_at Cluster Incl. AL050297:Homo sapiens mRNA; cDNA DKFZp564N123 (from clone; 35170_at Cluster Incl. AF044414:Homo sapiens alpha mannosidase 6A8B (6a8b) mRNA,; 35227_at Cluster Incl. U72066:Homo sapiens CtBP interacting protein CtIP (CtIP); 35620_at Cluster Incl. AF043250:Homo sapiens mitochondrial outer membrane protei; 35634_at Cluster Incl. U49928:Homo sapiens TAK1 binding protein (TAB1) mRNA, com; 36027_at Cluster Incl. AA418779:zv98d05.r1 Homo sapiens cDNA, 5 end /clone=IMAG; 36076_g_at Cluster Incl. AL037167:DKFZp564P1564_s1 Homo sapiens cDNA, 3 end /cl; 36475_at Cluster Incl. Z97630:Human DNA sequence from clone 466N1 on chromosome; 36870_at Cluster Incl. AB018347:Homo sapiens mRNA for KIAA0804 protein, partial; 37992_s_at Cluster Incl. AI436567:ti03b09.x1 Homo sapiens cDNA, 3 end /clone=IM; 40807_at Cluster Incl. X86018:H. sapiens mRNA for MUF1 protein /cds=(0,1853) /gb=; 40875_s_at Cluster Incl. X06815:Human mRNA for hU1-70K small nuclear RNP protein; 32145_at Cluster Incl. X58141:Human mRNA for erythrocyte adducin alpha subunit /; 32235_at Cluster Incl. AB011116:Homo sapiens mRNA for KIAA0544 protein, partial; 32758_g_at Cluster Incl. U84720:Homo sapiens mRNA export protein (RAE1) mRNA, co; 33441_at Cluster Incl. L41143:Homo sapiens expressed pseudo TCTA mRNA at t(1;3) 34315_at Cluster Incl. Y18314:Homo sapiens mRNA for paraplegin-like protein /cds; 35262_at Cluster Incl. AF022229:Homo sapiens translation initiation factor 6 (eI; 35758_at Cluster Incl. AB024301:Homo sapiens mRNA for RuvB-like DNA helicase TIP; 35826_at Cluster Incl. AF040253:Homo sapiens transcription factor Tat-CT1 mRNA,; 36154_at Cluster Incl. D87452:Human mRNA for KIAA0263 gene, complete cds /cds=(3; 36172_s_at Cluster Incl. AF002163:Homo sapiens delta-adaptin mRNA, complete cds; 36194_at Cluster Incl. M63959:Human alpha-2-macroglobulin receptor-associated pr; 36624_at Cluster Incl. L33842:Homo sapiens (clone FFE-7) type II inosine monopho; 36645_at Cluster Incl. L19067:Human NF-kappa-B transcription factor p65 subunit; 36973_at Cluster Incl. U41371:Human spliceosome associated protein (SAP 145) mRN; 37376_at Cluster Incl. M68864:Human ORF mRNA, complete cds /cds=(135,1031) /gb=M; 37378_r_at Cluster Incl. M13452:Human lamin A mRNA, 3end /cds=(0,1547) /gb=M1345; 38758_at Cluster Incl. R98910:yr31d04.r1 Homo sapiens cDNA, 5 end /clone=IMAGE-; 39089_at Cluster Incl. Y07604:H. sapiens mRNA for nucleoside-diphosphate kinase /; 39130_at Cluster Incl. AB018313:Homo sapiens mRNA for KIAA0770 protein, partial; 39149_at Cluster Incl. X99720:H. sapiens TPRC gene /cds=(212,1687) /gb=X99720 /gi; 39180_at Cluster Incl. S62140:TLS=translocated in liposarcoma [human, mRNA, 1824; 40182_s_at Cluster Incl. AF055027:Homo sapiens clone 24658 mRNA sequence /cds=UN; 40223_r_at Cluster Incl. AI677689:wd33c06.x1 Homo sapiens cDNA, 3 end /clone=IM; 40264_g_at Cluster Incl. AF001891:Homo sapiens clone lambda MEN1 region unknown; 41316_s_at Cluster Incl. U72355:Human Hsp27 ERE-TATA-binding protein (HET) mRNA,; 41527_f_at Cluster Incl. W29063:56g11 Homo sapiens cDNA /gb=W29063 /gi=1309092 /; 41590_at Cluster Incl. AI652660:wb30c10.x1 Homo sapiens cDNA, 3 end /clone=IMAG; 1310_at D26599 /FEATURE= /DEFINITION=HUMPSH2 Human mRNA for proteasome subunit H; 1000_at X60188 /FEATURE=mRNA /DEFINITION=HSERKI Human ERK1 mRNA for protein seri; 410_s_at X57152 /FEATURE=mRNA#1 /DEFINITION=HSCKIIBE Human gene for casein kinas; 162_at U44839 /FEATURE= /DEFINITION=HSU44839 Human putative ubiquitin C-terminal; 109_at Z97074 /FEATURE= /DEFINITION=HSRAB9P40 Homo sapiens mRNA for Rab9 effecto.

Metagene 347; 41656_at Cluster Incl. AF043325:Homo sapiens N-myristoyltransferase 2 mRNA, comp; 41691_at Cluster Incl. AB018337:Homo sapiens mRNA for KIAA0794 protein, partial; 32143_at Cluster Incl. AI126171:qd81g02.x1 Homo sapiens cDNA, 3 end /clone=IMAG; 35717_at Cluster Incl. AB020629:Homo sapiens mRNA for KIAA0822 protein, complete; 37616_at Cluster Incl. X79888:H. sapiens AUH mRNA /cds=(4,1023) /gb=X79888 /gi=78; 40423_at Cluster Incl. AB020710:Homo sapiens mRNA for KIAA0903 protein, partial; 40775_at Cluster Incl. AL021786:Human DNA sequence from PAC 696H22 on chromosome; 41229_at Cluster Incl. AI222594:qg58g05.x1 Homo sapiens cDNA, 3 end /clone=IMAG; 32747_at Cluster Incl. X05409:Human RNA for mitochondrial aldehyde dehydrogenase; 34821_at Cluster Incl. AL050197:Homo sapiens mRNA; cDNA DKFZp586D0623 (from clon; 35327_at Cluster Incl. U54559:Homo sapiens translation initiation factor eIF3 p4; 37348_s_at Cluster Incl. AA845349:ak01g01.s1 Homo sapiens cDNA, 3 end /clone=IM; 38051_at Cluster Incl. X76220:H. sapiens MAL gene exon 1 (and joined CDS) /cds=(5; 38098_at Cluster Incl. D80010:Human mRNA for KIAA0188 gene, partial cds /cds=(0,; 39110_at Cluster Incl. X55733:H. sapiens initiation factor 4B cDNA /cds=(0,1835); 41309_g_at Cluster Incl. U37408:Homo sapiens phosphoprotein CtBP mRNA, complete; 41796_at Cluster Incl. AB029015:Homo sapiens mRNA for KIAA1092 protein, partial; 33140_at Cluster Incl. AF029893:Homo sapiens i-beta-1,3-N-acetylglucosaminyltran; 1897_at L07594 /FEATURE= /DEFINITION=HUMTGFB3C Human transforming growth factor-; 1787_at U22398 /FEATURE= /DEFINITION=HSU22398 Human Cdk-inhibitor p57KIP2 (KIP2); 103_at Z19585 /FEATURE=cds /DEFINITION=HSTHROMB4 H. sapiens mRNA for thrombospond.

Metagene 348; 35599_at Cluster Incl. X62250:H. sapiens mRNA for liver glycine methyltransferase; 32351_at Cluster Incl. U66579:Human putative G protein-coupled receptor (GPR20); 36729_g_at Cluster Incl. M76446:Human alpha-A1-adrenergic receptor mRNA, complet; 37916_at Cluster Incl. AI086057:oz44f07.x1 Homo sapiens cDNA, 3 end /clone=IMAG; 1730_s_at M17446 /FEATURE=mRNA /DEFINITION=HUMKSGFA Human Kaposi s sarcoma oncog; 910_at M15205 /FEATURE=cds /DEFINITION=HUMTKRA Human thymidine kinase gene, comp.

Metagene 349; 31514_at Cluster Incl. AF034970:Homo sapiens docking protein (DOK-2) mRNA, compl; 31596_f_at Cluster Incl. L02326:Homo sapiens (clone Hu lambda-17) lambda-like ge; 35017_f_at Cluster Incl. M80469:Human MHC class I HLA-J gene, exons 1-8 and comp; 33967_at Cluster Incl. M31525:Human MHC class II lymphocyte antigen (HLA-DNA) ge; 36731_g_at Cluster Incl. U66684:HSU66684 Homo sapiens cDNA /gb=U66684 /gi=190656; 37419_g_at Cluster Incl. M36653:Human Oct-2 factor mRNA, complete cds /cds=UNKNO; 37421_f_at Cluster Incl. AL022723:dJ377H14.9 (major histocompatibility complex,; 37769_at Cluster Incl. AF011466:Homo sapiens G protein-coupled receptor Edg-4 mR; 37779_at Cluster Incl. Y08134:H. sapiens mRNA for ASM-like phosphodiesterase 3b /; 37790_at Cluster Incl. AF068006:Homo sapiens haemopoietic progenitor homeobox HP; 37843_i_at Cluster Incl. AF053004:Homo sapiens class I cytokine receptor (WSX1); 38149_at Cluster Incl. D29642:Human mRNA for KIAA0053 gene, complete cds /cds=(1; 38963_i_at Cluster Incl. U12707:Human Wiskott-Aldrich syndrome protein (WASP) mR; 39568_g_at Cluster Incl. AB006190:Homo sapiens mRNA for aquaporin adipose, compl; 40718_at Cluster Incl. AF013611:Homo sapiens lymphopain mRNA, complete cds /cds=; 41423_at Cluster Incl. AB018269:Homo sapiens mRNA for KIAA0726 protein, complete; 32629_f_at Cluster Incl. U90552:Human butyrophilin (BIT5) mRNA, complete cds /cd; 35150_at Cluster Incl. X60592:Human CDw40 mRNA for nerve growth factor receptor-; 35197_at Cluster Incl. AF038188:Homo sapiens clone 23940 mRNA sequence /cds=UNKN; 39008_at Cluster Incl. M13699:Human ceruloplasmin (ferroxidase) mRNA, complete c; 39778_at Cluster Incl. M55621:Human N-acetylglucosaminyltransferase I (GlcNAc-TI; 40836_s_at Cluster Incl. W26677:11f7 Homo sapiens cDNA /gb=W26677 /gi=1305788 /u; 40840_at Cluster Incl. M80254:H. sapiens cyclophilin isoform (hCyP3) mRNA, comple; 33390_at Cluster Incl. AA203487:zx53d03.r1 Homo sapiens cDNA, 5 end /clone=IMAG; 33448_at Cluster Incl. AB000095:Homo sapiens mRNA for hepatocyte growth factor a; 34835_at Cluster Incl. D87442:Human mRNA for KIAA0253 gene, partial cds /cds=(0,; 35309_at Cluster Incl. U20428:Human SNC19 mRNA sequence /cds=UNKNOWN /gb=U20428; 35798_at Cluster Incl. W25936:15b5 Homo sapiens cDNA /gb=W25936 /gi=1306059 /ug=; 37746_r_at Cluster Incl. U15131:Human p126 (ST5) mRNA, complete cds /cds=(114,35; 38121_at Cluster Incl. X59892:H. sapiens mRNA for IFN-inducible gamma2 protein/c; 38445_at Cluster Incl. Y09160:H. sapiens Sub1.5 mRNA /cds=(435,3044) /gb=Y09160 /; 41565_at Cluster Incl. AF034373:Homo sapiens ataxin-2-like protein A2LP (A2LG) m; 2090_i_at H12458 /FEATURE= /DEFINITION=H12458 yj12d03.s1 Soares placenta Nb2HP H; 1796_s_at U05681 /FEATURE=expanded_cds /DEFINITION=HSBCL3S2 Human proto-oncogene; 1291_s_at L03840 /FEATURE= /DEFINITION=HUMFGFR4X Human fibroblast growth factor; 993_at X54637 /FEATURE=cds /DEFINITION=HSTYK2 Human tyk2 mRNA for non-receptor p; 810_at U64105 /FEATURE= /DEFINITION=HSU64105 Human guanine nucleotide exchange f; 160042_s_at X58431 /FEATURE=mRNA#1 /DEFINITION=HSHOX22 Human Hox2.2 gene for a h.

Metagene 350; 36437_s_at Cluster Incl. AF064606:Homo sapiens KB07 protein mRNA, partial cds /c; 36213_at Cluster Incl. AB016816:Homo sapiens MASL1 mRNA, complete cds /cds=(0,31; 36238_at Cluster Incl. Y11284:Homo sapiens AFX1 gene, exon 1 (and joined CDS) /c; 37134_f_at Cluster Incl. L13266:Homo sapiens N-methyl-d-aspartate receptor (NR1-; 37138_at Cluster Incl. AB018352:Homo sapiens mRNA for KIAA0809 protein, partial; 37443_at Cluster Incl. AL031588:dJ1163J1.2.1 (novel protein similar to C. elegan; 37445_at Cluster Incl. AB015633:Homo sapiens mRNA for type II membrane protein,; 38569_at Cluster Incl. U02683:Human alpha palindromic binding protein mRNA, comp; 39246_at Cluster Incl. Z75330:H. sapiens mRNA for nuclear protein SA-1 /cds=(400,; 39266_at Cluster Incl. AF070632:Homo sapiens clone 24405 mRNA sequence /cds=UNKN; 31824_at Cluster Incl. AL049699:dJ747H23.1 (malic enzyme 1, soluble (NADP-depend; 33319_at Cluster Incl. AF009674:Homo sapiens axin (AXIN) mRNA, partial cds /cds=; 33791_at Cluster Incl. Y15227:Homo sapiens mRNA for leukemia associated gene 1 /; 37617_at Cluster Incl. U90912:Human clone 23865 mRNA sequence /cds=UNKNOWN /gb=U; 37907_at Cluster Incl. M34677:Human nested gene protein gene, complete cds /cds=; 38295_at Cluster Incl. X59842:Human PBX2 mRNA /cds=UNKNOWN /gb=X59842 /gi=35312; 38662_at Cluster Incl. AL047596:DKFZp586G0421_s1 Homo sapiens cDNA /clone=DKFZp5; 39337_at Cluster Incl. M37583:Human histone (H2A.Z) mRNA, complete cds /cds=(106; 39708_at Cluster Incl. L29277:Homo sapiens DNA-binding protein (APRF) mRNA, comp; 39719_at Cluster Incl. X56777:H. sapiens mRNA for ZP3 gene /cds=(30,1148) /gb=X56; 39734_at Cluster Incl. U10117:Human endothelial-monocyte activating polypeptide; 39788_at Cluster Incl. X81889:H. sapiens mRNA for p0071 protein /cds=(141,3776) /; 39791_at Cluster Incl. M23114:Homo sapiens calcium-ATPase (HK1) mRNA, complete c; 40487_at Cluster Incl. W26634:34b10 Homo sapiens cDNA /gb=W26634 /gi=1307477 /ug; 40771_at Cluster Incl. Z98946:Human DNA sequence from clone 376D21 on chromosome; 40813_at Cluster Incl. AI768188:wg82b12.x1 Homo sapiens cDNA, 3 end /clone=IMAG; 40881_at Cluster Incl. X64330:H. sapiens mRNA for ATP-citrate lyase /cds=(84,3401; 33434_at Cluster Incl. AF007551:Homo sapiens Bet1p homolog (hbet1) mRNA, complet; 34831_at Cluster Incl. AF055004:Homo sapiens clone 24640 mRNA sequence /cds=UNKN; 36146_at Cluster Incl. AF057297:Homo sapiens ornithine decarboxylase antizyme 2; 36954_at Cluster Incl. D86972:Human mRNA for KIAA0218 gene, complete cds /cds=(3; 37050_r_at Cluster Incl. AI130910:qb81g08.x1 Homo sapiens cDNA, 3 end /clone=IM; 37292_at Cluster Incl. D83785:Human mRNA for KIAA0200 gene, complete cds /cds=(2; 37393_at Cluster Incl. L19314:Human HRY gene, complete cds /cds=(0,842) /gb=L193; 38083_at Cluster Incl. AL049386:Homo sapiens mRNA; cDNA DKFZp586M0918 (from clon; 38415_at Cluster Incl. U14603:Human protein-tyrosine phosphatase (HU-PP-1) mRNA,; 40279_at Cluster Incl. D50911:Human mRNA for KIAA0121 gene, complete cds /cds=(4; 1674_at M15990 /FEATURE= /DEFINITION=HUMCYES1 Human c-yes-1 mRNA; 1507_s_at D11151 /FEATURE=_expandCDS /DEFINITION=HUMETAR8 Human DNA for endothe1; 1031_at U09564 /FEATURE= /DEFINITION=HSU09564 Human serine kinase mRNA, complete; 914_g_at M21535 /FEATURE= /DEFINITION=HUMERG11 Human erg protein (ets-related ge.

Metagene 351; 32290_at Cluster Incl. AL049223:Homo sapiens mRNA; cDNA DKFZp564L1916 (from clon; 40686_at Cluster Incl. AI985272:ws06b05.x1 Homo sapiens cDNA, 3 end /clone=IMAG; 36673_at Cluster Incl. X76057:H. sapiens PMI1 mRNA for phosphomannose isomerase /; 37353_g_at Cluster Incl. M60618:Human nuclear autoantigen (SP-100) mRNA, complet; 39114_at Cluster Incl. AB022718:Homo sapiens mRNA for DEPP (decidual protein ind; 41322_s_at Cluster Incl. AI816034:au44e05.x1 Homo sapiens cDNA, 3 end /clone=IM; 1043_s_at U27516 /FEATURE= /DEFINITION=HSU27516 Human recombination protein RAD5; 895_at L19686 /FEATURE=mRNA /DEFINITION=HUMMIF Homo sapiens macrophage migration; 849_g_at U19261 /FEATURE= /DEFINITION=HSU19261 Homo sapiens Epstein-Barr virus-i.

Metagene 352; 34498_at Cluster Incl. D89974:Homo sapiens mRNA for glycosylphosphatidyl inosito; 37842_at Cluster Incl. AF054589:Homo sapiens HIC protein mRNA, complete cds /cds; 39624_at Cluster Incl. D89078:Homo sapiens mRNA for leukotriene b4 receptor, com; 36924_r_at Cluster Incl. M25756:Human secretogranin II gene, complete cds /cds=(; 41783_at Cluster Incl. M97815:Human retinoic acid-binding protein II (CRABP-II); 32250_at Cluster Incl. X07523:Human mRNA for truncated form of complement factor; 40553_at Cluster Incl. AI742087:wg38g10.x1 Homo sapiens cDNA, 3 end /clone=IMAG; 40938_at Cluster Incl. Y13835:Homo sapiens mRNA for farnesylated-proteins conver; 33192_g_at Cluster Incl. AW051579:wy87g03.x1 Homo sapiens cDNA, 3 end /clone=IM; 1932_at U83661 /FEATURE= /DEFINITION=HSU83661 Homo sapiens multidrug resistance; 1573_at M12783 /FEATURE=mRNA /DEFINITION=HUMSISPDG Human c-sis/platelet-derived.

Metagene 353; 36229_at Cluster Incl. U58917:Homo sapiens IL-17 receptor mRNA, complete cds /cd; 38581_at Cluster Incl. U40038:Human GTP-binding protein alpha q subunit (GNAQ) m; 32178_r_at Cluster Incl. AJ011915:Homo sapiens mRNA for synaptosome associated p; 33391_r_at Cluster Incl. S57235:CD68=110 kda transmembrane glycoprotein [human, p; 39525_at Cluster Incl. AL120687:DKFZp762F2110_r1 Homo sapiens cDNA, 5 end /clon; 40613_at Cluster Incl. AL031775:dJ30M3.2 (novel protein) /cds=(0,260) /gb=AL0317; 41809_at Cluster Incl. AI656421:tt50h10.x1 Homo sapiens cDNA, 3 end /clone=IMAG.

Metagene 354; 39986_at Cluster Incl. AL050100:Homo sapiens mRNA; cDNA DKFZp586D0919 (from clon; 41389_s_at Cluster Incl. U46193:Human renal cell carcinoma antigen RAGE-3 mRNA,; 41708_at Cluster Incl. AB028957:Homo sapiens mRNA for KIAA1034 protein, partial; 36485_at Cluster Incl. U85647:Homo sapiens small optic lobes homolog (SOLH) mRNA; 36488_at Cluster Incl. AB011542:Homo sapiens mRNA for MEGF9, partial cds /cds=(0; 38264_at Cluster Incl. U74324:Human guanine nucleotide exchange factor mss4 mRNA; 38272_at Cluster Incl. AF038844:Homo sapiens MKP-1 like protein tyrosine phospha; 38336_at Cluster Incl. AB023230:Homo sapiens mRNA for KIAA1013 protein, partial; 38988_at Cluster Incl. AJ007042:Homo sapiens mRNA for TRX5 protein /cds=(762,485; 39695_at Cluster Incl. M31516:Human decay-accelerating factor mRNA, complete cds; 32174_at Cluster Incl. AF015926:Homo sapiens ezrin-radixin-moesin binding phosph; 32807_at Cluster Incl. AF004292:Homo sapiens clone IMAGE-30181 mRNA sequence /cd; 33917_at Cluster Incl. AB002336:Human mRNA for KLAA0338 gene, partial cds /cds=(; 40238_at Cluster Incl. AI674208:wc07f02.x1 Homo sapiens cDNA, 3 end /clone=IMAG; 41816_at Cluster Incl. AL049851:Human DNA sequence from clone 889J22B on chromos; 33158_at Cluster Incl. M97252:Homo sapiens Kallmann syndrome (KAL) mRNA, complet.

Metagene 355; 31737_at Cluster Incl. J00068:Human adult skeletal muscle alpha-actin mRNA /cds=; 32432_f_at Cluster Incl. L25899:Human ribosomal protein L10 mRNA, complete cds /; 33621_at Cluster Incl. X71348:Homo sapiens vHNF1-C mRNA /cds=UNKNOWN /gb=X71348; 33637_g_at Cluster Incl. U87459:Human autoimmunogenic cancer/testis antigen NY-E; 33689_-sat Cluster Incl. AF012434:untitled /cds-(38,394) /gb=AF012434 /gi=235291; 34085_at Cluster Incl. Z26876:H. sapiens gene for ribosomal protein L38 /cds=(110; 34618_at Cluster Incl. L38517:Homo sapiens indian hedgehog protein (IHH) mRNA, 5; 35078_at Cluster Incl. X93093:H. sapiens LW gene /cds=(9,824) /gb=X93093 /gi=1491; 35471_g_at Cluster Incl. AA418537:zv93a09.s1 Homo sapiens cDNA, 3 end /clone=IM; 37529_at Cluster Incl. AF051946:Homo sapiens T-type calcium channel alpha-1 subu; 38904_at Cluster Incl. AF077754:Homo sapiens tyrosine kinase pp60c-src (SRC) gen; 40755_at Cluster Incl. X92841:H. sapiens MICA gene /cds=(39,1196) /gb=X92841 /gi=; 41114_at Cluster Incl. AB018350:Homo sapiens mRNA for KIAA0807 protein, partial; 41603_at Cluster Incl. U02609:Human transducin-like protein mRNA, complete cds /; 32091_at Cluster Incl. AB007915:Homo sapiens mRNA for KIAA0446 protein, complete; 33337_at Cluster Incl. AF002668:Homo sapiens putative fatty acid desaturase MLD; 34245_at Cluster Incl. W25917:14h6 Homo sapiens cDNA /gb=W25917 /gi=1306040 /ug=; 34295_at Cluster Incl. AB011101 Homo sapiens mRNA for KIAA0529 protein, partial; 35183_at Cluster Incl. U78735:Human ABC3 mRNA, complete cds /cds=(559,5673) /gb=; 35653_at Cluster Incl. U28963:Human Gps2 (GPS2) mRNA, complete cds /cds=(90,1073; 36930_at Cluster Incl. L05425:Homo sapiens autoantigen mRNA, complete cds /cds=(; 37888_at Cluster Incl. D87449:Human mRNA for KIAA0260 gene, partial cds /cds=(0,; 38688_at Cluster Incl. AB007930:Homo sapiens mRNA for KIAA0461 perotein, partial; 38695_at Cluster Incl. AA203303:zx55b01.r1 Homo sapiens cDNA, 5 end /clone=IMAG; 39029_at Cluster Incl. U11861 Human G10 homolog (edg-2) mRNA, complete cds /cds=; 39688_at Cluster Incl. AF001433:Human requiem (HREQ) mRNA, complete cds /cds=(41; 39723_at Cluster Incl. AF062536:Homo sapiens cullin 1 mRNA, complete cds /cds=(1; 39800_s_at Cluster Incl. U68566:Human HS1 binding protein HAX-1 mRNA, nuclear ge; 40130_at Cluster Incl. U06863:Human follistatin-related protein precursor mRNA,; 40870_g_at Cluster Incl. AF069517:Homo sapiens RNA binding protein DEF-3 mRNA, c; 33915_at Cluster Incl. W22655:71B9 Homo sapiens cDNA /clone=(not-directional) /g; 34302_at Cluster Incl. U96074:Human translation initiation factor eIF3 p44 subun; 34325_at Cluster Incl. AJ005893:Homo sapiens mRNA for JM26 protein, complete CDS; 36093_at Cluster Incl. AB014514:Homo sapiens mRNA for KIAA0614 protein, partial; 36185_at Cluster Incl. D32050:Human mRNA for alanyl-tRNA synthetase, complete cd; 40276_at Cluster Incl. D50063:Human mRNA for proteasome subunit p40_/Mov34 prot; 40609_at Cluster Incl. AI475497:tj92g12.x1 Homo sapiens cDNA, 3 end /clone=IMAG; 40937_at Cluster Incl. AA160056:zo84f11.s1 Homo sapiens cDNA, 3 end /clone=IMAG; 41272_s_at Cluster Incl. W29031:55c6 Homo sapiens cDNA /gb=W29031 /gi=1308988 /u; 41821_at Cluster Incl. AA203246:zx54h03.r1 Homo sapiens cDNA, 5 end /clone=IMAG; 32546_at Cluster Incl. U59309:Human fumarase precursor (FH) mRNA, nuclear gene e; 33162_at Cluster Incl. X02160:Human mRNA for insulin receptor precursor /cds=(48; 1875_f_at D38498 /FEATURE= /DEFINITION=HUMPMS1A Human PMS5 mRNA (yeast mismatch; 1808_s_at X89101 /FEATURE=cds /DEFINITION=HSFASMRNA H. sapiens mRNA for Fas (Apo-; 1409_at J03161 /FEATURE=mRNA /DEFINITION=HUMSRF Human serum response factor (SRF; 541_g_at S67070 /FEATURE= /DEFINITION=S67070 heat shock protein HSP72 homolog [h.

Metagene 356; 37471_at Cluster Incl. U94317:Homo sapiens ribonuclease P protein subunit p40 (R; 38151_at Cluster Incl. AF002672:Homo sapiens breast cancer suppressor candidate; 39618_at Cluster Incl. U43328:Human link protein mRNA, complete cds /cds=(117,11; 39961_at Cluster Incl. AB011138:Homo sapiens mRNA for KIAA0566 protein, partial; 41470_at Cluster Incl. AF027208:Homo sapiens AC133 antigen mRNA, complete cds /c.

Metagene 357; 35075_at Cluster Incl. AI571208:tn43d09.x1 Homo sapiens cDNA, 3 end /clone=IMAG; 39619_at Cluster Incl. AF070551:Homo sapiens clone 24515 mRNA sequence /cds=UNKN; 39940_at Cluster Incl. AL080094:Homo sapiens mRNA; cDNA DKFZp564O1262 (from clon; 41029_at Cluster Incl. U44798:Human U1-snRNP binding protein homolog mRNA, compl; 41402_at Cluster Incl. AL080121:Homo sapiens mRNA; cDNA DKFZp564O0823 (from clon; 36525_at Cluster Incl. AL049953:Homo sapiens mRNA; cDNA DKFZp564P0622 (from clon; 38975_at Cluster Incl. AF062534:Homo sapiens genethonin 1 mRNA, complete cds /cd; 39017_at Cluster Incl. AJ238094:Homo sapiens mRNA for Lsm1 protein /cds=(188,589; 39702_at Cluster Incl. U77665:Human RNaseP protein p30 (RPP30) mRNA, complete cd; 34400_at Cluster Incl. AI540957:PEC1.2_{—}15_G03.r Homo sapiens cDNA, 5 end /clone; 35288_at Cluster Incl. AB007884:Homo sapiens KIAA0424 mRNA, partial cds /cds=(80; 37312_at Cluster Incl. D50917:Human mRNA for KIAA0127 gene, complete cds /cds=(2; 869_at U14193 /FEATURE= /DEFINITION=HSU14193 Human TFIIA gamma subunit mRNA, com; 283_at L16842 /FEATURE= /DEFINITION=HUMMITCORA Human ubiquinol cytochrome-c redu.

Metagene 358; 35956_s_at Cluster Incl. U18467:Human pregnancy-specific beta 1-glycoprotein 7 (; 37102_at Cluster Incl. AA203717:zx52f12.r1 Homo sapiens cDNA, 5 end /clone=IMAG; 39496_s_at Cluster Incl. W27595:34h5 Homo sapiens cDNA /gb=W27595 /gi=1307543 /u; 40241_at Cluster Incl. U09850:Human zinc finger protein (ZNF143) mRNA, complete; 41518_at Cluster Incl. AI925356:wn53b06.x1 Homo sapiens cDNA, 3 end /clone=IMAG; 601_s_at M28439 /FEATURE=cds /DEFINITION=HUMKER16A8 Human keratin type 16 gene,.

Metagene 359; 32977_at Cluster Incl. U49187:Human placenta (Diff48) mRNA, complete cds /cds=(4; 34898_at Cluster Incl. M30704:Human amphiregulin (AR) mRNA, complete cds, clones; 41870_at Cluster Incl. AF030428:Homo sapiens lung type-I cell membrane-associate; 37544_at Cluster Incl. X64318:H. sapiens E4BP4 gene /cds=(213,1601) /gb=X64318 /g; 38354_at Cluster Incl. X52560:Human gene for nuclear factor NF-IL6 /cds=(0,1037); 39081_at Cluster Incl. AI547258:PN001_AH_H08.r Homo sapiens cDNA, 5 end /clone_{—}; 36634_at Cluster Incl. U72649:Human BTG2 (BTG2) mRNA, complete cds /cds=(71,547); 38805_at Cluster Incl. X89750:H. sapiens mRNA for TGIF protein /cds=(311,1129) /g; 39822_s_at Cluster Incl. AF078077:Homo sapiens growth arrest and DNA-damage-indu; 40268_at Cluster Incl. X16706:Human fra-2 mRNA /cds=(3,983) /gb=X16706 /gi=31464; 33146_at Cluster Incl. L08246:Human myeloid cell differentiation protein (MCL1) 1052_s_at M83667 /FEATURE=mRNA /DEFINITION=HUMNFIL6BA Human NF-IL6-beta protein; 998_s_at X59770 /FEATURE=mRNA /DEFINITION=HSIL1R2II H. sapiens IL-1R2 mRNA for ty; 277_at L08246 /FEATURE= /DEFINITION=HUMMCL1X Human myeloid cell differentiation.

Metagene 360; 31982_at Cluster Incl. AB020701:Homo sapiens mRNA for KIAA0894 protein, complete; 32986_s_at Cluster Incl. D83760:Homo sapiens mRNA for mother against dpp (Mad) r; 34162_at Cluster Incl. D84111:Homo sapiens mRNA for RBP-MS/type 5, partial cds /; 34163_g_at Cluster Incl. D84111:Homo sapiens mRNA for RBP-MS/type 5, partial cds; 36691_at Cluster Incl. X82224:H. sapiens mRNA for glutamine transaminase K /cds=(; 41028_at Cluster Incl. AJ001515:Homo sapiens mRNA for ryanodine receptor 3, comp; 33290_at Cluster Incl. M74161:Human inositol polyphosphate 5-phosphatase (5ptase; 33808_at Cluster Incl. AL022721:dJ109F14.1.1 (Transcriptional Enhancer Factor TE; 34203_at Cluster Incl. D17408:Homo sapiens mRNA for calponin, complete cds /cds=; 35211_at Cluster Incl. L07590:Human protein phosphatase 2A 130 kDa regulatory su; 37580_at Cluster Incl. AF036271:Homo sapiens EEN-B2-L3 mRNA, complete cds /cds=(; 38298_at Cluster Incl. U25138:Human MaxiK potassium channel beta subunit mRNA, c; 38984_at Cluster Incl. AB007896:Homo sapiens KIAA0436 mRNA, partial cds /cds=(0,; 39730_at Cluster Incl. X16416:Human c-abl mRNA encoding p150 protein /cds=(147,3; 41195_at Cluster Incl. U49957:Human LIM protein (LPP) mRNA, partial cds /cds=(24; 32856_at Cluster Incl. AB020626:Homo sapiens mRNA for KIAA0819 protein, partial; 35330_at Cluster Incl. AJ012737:Homo sapiens mRNA for filamin, muscle isoform /c; 35359_at Cluster Incl. D87078:Human mRNA for KIAA0235 gene, partial cds /cds=(1,; 37407_s_at Cluster Incl. AF013570:Homo sapiens smooth muscle myosin heavy chain; 38048_at Cluster Incl. D84110:Homo sapiens mRNA for RBP-MS/type 4, complete cds; 38049_g_at Cluster Incl. D84110:Homo sapiens mRNA for RBP-MS/type 4, complete cd; 40165_at Cluster Incl. AB015345:Homo sapiens HRIHFB2216 mRNA, partial cds /cds=(; 40578_s_at Cluster Incl. M77016:Human tropomodulin mRNA, complete cds /cds=(34,1; 1826_at M12174 /FEATURE= /DEFINITION=HUMRHOA Human ras-related rho mRNA (clone 6; 1635_at U07563 /FEATURE=Poly_A_Site#1 /DEFINITION=HSABLGR3 Human proto-oncogene; 1297_at D84110 /FEATURE= /DEFINITION=D84110 Homo sapiens mRNA for RBP-MS/type 4,; 1276_g_at D84110 /FEATURE= /DEFINITION=D84110 Homo sapiens mRNA for RBP-MS/type; 1197_at D00654 /FEATURE=cds /DEFINITION=HUMACTSG7 Homo sapiens gene for enteric; 343_s_at D12485 /FEATURE=cds# 1 /DEFINITION=HUMNPP Human mRNA for nucleotide pyro.

Metagene 361; 33602_at Cluster Incl. AJ000479:Homo sapiens mRNA for putative G-protein coupled; 34122_at Cluster Incl. AF027807:Homo sapiens beta-casein (CSN2) gene, complete c; 35381_at Cluster Incl. AL080127:Homo sapiens mRNA; cDNA DKFZp434C013 (from clone; 37792_s_at Cluster Incl. Y15572:Homo sapiens mRNA for recombination repair prote; 38202_at Cluster Incl. AB011535:Homo sapiens mRNA for MEGF1, partial cds /cds=(0; 38899_s_at Cluster Incl. U95822:Human putative transmembrane GTPase mRNA, partia; 40371_at Cluster Incl. X51362:Human mRNA for dopamine D2 receptor /cds=(165,1496; 35188_at Cluster Incl. AB018336:Homo sapiens mRNA for KIAA0793 protein, complete; 36479_at Cluster Incl. AF050078:untitled /cds=(122,1558) /gb=AF050078 /gi=381846; 38623_at Cluster Incl. AI014538:ou40e01.x1 Homo sapiens cDNA, 3 end /clone=IMAG; 39738_at Cluster Incl. Z82215:Homo sapiens DNA sequence from PAC 6802 on chromos; 41170_at Cluster Incl. AB014563:Homo sapiens mRNA for KIAA0663 protein, complete; 34829_at Cluster Incl. U59151:Human Cbf5p homolog (CBF5) mRNA, complete cds /cds; 38779_r_at Cluster Incl. D16431:Human mRNA for hepatoma-derived growth factor, c; 41262_at Cluster Incl. AL021707:Human DNA sequence from clone 508115 on chromoso; 1662_r_at Antigen, Prostate Specific, Alt. Splice Form 2; 1366_i_at M26880 /FEATURE= /DEFINITION=HUMUBI13 Human ubiquitin mRNA, complete c; 258_at M16441 /FEATURE=cds#1 /DEFINITION=HUMTNFAB Human tumor necrosis factor an.

Metagene 362; 31486_s_at Cluster Incl. Z70218:H. sapiens mRNA for MNI protein (clone ICRFp507I0; 31943_g_at Cluster Incl. AF045583:Homo sapiens tubby like protein 3 (TULP3) mRNA; 33533_at Cluster Incl. U40992:Homo sapiens heat shock protein hsp40 homolog mRNA; 36718_s_at Cluster Incl. L42452:Homo sapiens pyruvate dehydrogenase kinase isoen; 36783_f_at Cluster Incl. M55422:Human Krueppel-related zinc finger protein (H-pl; 39565_at Cluster Incl. Z22535:H. sapiens ALK-3 mRNA /cds=(309,1907) /gb=Z22535 /g; 40387_at Cluster Incl. U80811:Human lysophosphatidic acid receptor homolog mRNA,; 34732_at Cluster Incl. X65873:H. sapiens mRNA for kinesin (heavy chain) /cds=(313; 34735_at Cluster Incl. U43195:Human Rho-associated, coiled-coil containing prote; 35221_at Cluster Incl. X91648:H. sapiens mRNA for pur alpha extended 3untranslate; 36534_at Cluster Incl. AF070621:Homo sapiens clone 24760 mRNA sequence /cds=UNKN; 37902_at Cluster Incl. L13278:Homo sapiens zeta-crystallin/quinone reductase mRN; 39055_at Cluster Incl. M32886:Human sorcin CP-22 mRNA, complete cds /cds=(12,608; 39365_i_at Cluster Incl. Y18207:Homo sapiens mRNA for protein phosphatase 1 (PPP; 39724_s_at Cluster Incl. U58087:Human Hs-cul-1 mRNA, complete cds /cds=(124,2382; 40119_at Cluster Incl. AJ006470:Homo sapiens mRNA for cartilage-associated prote; 40441_g_at Cluster Incl. AL080119:Homo sapiens mRNA; cDNA DKFZp564M2423 (from cl; 40879_at Cluster Incl. AB014599:Homo sapiens mRNA for KIAA0699 protein, partial; 41234_at Cluster Incl. AI540318:tq34f03.x1 Homo sapiens cDNA, 3 end /clone=IMAG; 41739_s_at Cluster Incl. M83216:Human aorta caldesmon mRNA, complete cds /cds=(2; 32179_s_at Cluster Incl. Y09568:Homo sapiens mRNA for SNAP23B protein, complete; 32779_s_at Cluster Incl. U23850:Human inositol 1,4,5 trisphosphate receptor type; 33385_g_at Cluster Incl. U31346:Human calpastatin mRNA, partial cds, long 3UTR /; 34350_at Cluster Incl. X64838:H. sapiens mRNA for restin /cds=(132,4415) /gb=X648; 35259_s_at Cluster Incl. Y11251:H. sapiens mRNA for novel member of serine-argini; 35788_at Cluster Incl. W28994:54h7 Homo sapiens cDNA /gb=W28994 /gi=1308960 /ug=; 36684_at Cluster Incl. M21154:Human S-adenosylmethionine decarboxylase mRNA, com; 37738_g_at Cluster Incl. D25547:Homo sapiens mRNA for PIMT isozyme I, complete c; 37747_at Cluster Incl. U05770:Human annexin V (ANX5) gene /cds=(164,1126) /gb=U0; 38736_at Cluster Incl. AL050108:Homo sapiens mRNA; cDNA DKFZp586I2219 (from clon; 38756_at Cluster Incl. M22995:Human ras-related protein (Krev-1) mRNA, complete; 41313_at Cluster Incl. M86917:Human oxysterol-binding protein (OSBP) mRNA, compl; 32548_at Cluster Incl. L24804:Human (p23) mRNA, complete cds /cds=(232,714) /gb=; 2085_s_at D14705 /FEATURE= /DEFINITION=HUMALPHAC Human mRNA for alpha-catenin, c; 2069_s_at L23805 /FEATURE= /DEFINITION=HUMCATENIN Human alpha1(E)-catenin mRNA,; 2033_s_at U10564 /FEATURE= /DEFINITION=HSU10564 Human CDK tyrosine 15-kinase WEE; 2010_at U33760 /FEATURE= /DEFINITION=HSU33760 Human cyclin A/CDK2-associated p19; 1959_at D88674 /FEATURE= /DEFINITION=D88674 Homo sapiens mRNA for antizyme inhib; 1423_at D78514 /FEATURE=cds /DEFINITION=D78514 Homo sapiens mRNA for ubiquitin-c; 772_at D10656 /FEATURE= /DEFINITION=HUMCRK Human mRNA for CRK-II, complete cds; 748_s_at D63940 /FEATURE= /DEFINITION=HUMHMXI1HL Human mRNA for Mxi1 protein, co; 621_at M28211 /FEATURE= /DEFINITION=HUMRAB4A Homo sapiens GTP-binding protein (R; 312_s_at Focal Adhesion Kinase; 227_g_at M33336 /FEATURE= /DEFINITION=HUMCAMPPK Human cAMP-dependent protein kin; 158_at U40992 /FEATURE= /DEFINITION=HSU40992 Homo sapiens heat shock protein hsp.

Metagene 363; 37523_at Cluster Incl. M74096:Human long chain acyl-CoA dehydrogenase (ACADL) mR; 40647_at Cluster Incl. Z32684:Homo sapiens mRNA for membrane transport protein (; 32709_at Cluster Incl. L39833:Homo sapiens (clone hKvBeta3) K+ channel beta subu; 3271_g_at Cluster Incl. X83127:H. sapiens mRNA for voltage gated potassium chann; 33799_at Cluster Incl. U76248:Human hSIAH2 mRNA, complete cds /cds=(526,1500) /g; 34257_at Cluster Incl. AB014605:Homo sapiens mRNA for KIAA0705 protein, complete; 34262_at Cluster Incl. Y15909:Homo sapiens mRNA for dia-156 protein /cds=(350,36; 35730_at Cluster Incl. X03350:Human mRNA for alcohol dehydrogenase beta-1-subuni; 38316_at Cluster Incl. AF052732:Homo sapiens 10-formyltetrahydrofolate dehydroge; 38717_at Cluster Incl. AL050159:Homo sapiens mRNA; cDNA DKFZp586A0522 (from clon; 39366_at Cluster Incl. N36638:yx88f05.r1 Homo sapiens cDNA, 5 end /clone=IMAGE-; 40141_at Cluster Incl. AB014595:Homo sapiens mRNA for KIAA0695 protein, complete; 40409_at Cluster Incl. U46689:Human microsomal aldehyde dehydrogenase (ALD10) mR; 40461_at Cluster Incl. AB007855:Homo sapiens KIAA0395 mRNA, partial cds /cds=(0,; 41770_at Cluster Incl. AA420624:nc61c12.r1 Homo sapiens cDNA /clone=IMAGE-745750; 41771_g_at Cluster Incl. AA420624:nc61c12.r1 Homo sapiens cDNA /clone=IMAGE-7457; 41772_at Cluster Incl. M68840:Human monoamine oxidase A (MAOA) mRNA, complete cd; 32251_at Cluster Incl. AA149307:zl25h05.s1 Homo sapiens cDNA, 3 end /clone=IMAG; 36119_at Cluster Incl. AF070648:Homo sapiens clone 24651 mRNA sequence /cds=UNKN; 38046_at Cluster Incl. AJ005579:Homo sapiens mRNA for Prer protein /cds=(0,1673); 38394_at Cluster Incl. D42047:Human mRNA for KIAA0089 gene, partial cds /cds=(0,; 39809_at Cluster Incl. AF019214:Homo sapiens HMG box containing protein 1 mRNA,; 41244_f_at Cluster Incl. X80910:H. sapiens PPP1CB mRNA /cds=(258,1241) /gb=X80910; 1686_g_at X82554 /FEATURE=mRNA /DEFINITION=HSSPHAR H. sapiens SPHAR gene for cycl; 1182_at D42108 /FEATURE= /DEFINITION=HUMPLCE Human mRNA for phospholipase C, com.

Metagene 364; 36239_at Cluster Incl. Z49194:H. sapiens mRNA for oct-binding factor /cds=(523,12; 37420_i_at Cluster Incl. AL022723:dJ377H14.9 (major histocompatibility complex,; 33304_at Cluster Incl. U88964:Human HEM45 mRNA, complete cds /cds=(37,582) /gb=U; 41166_at Cluster Incl. X58529:Human rearranged immunoglobulin mRNA for mu heavy; 34332_at Cluster Incl. D31766:Human mRNA for KIAA0060 gene, complete cds /cds=(4; 36599_at Cluster Incl. M55905:Human mitochondrial NAD(P)+ dependent malic enzyme; 40615_at Cluster Incl. AA780049:zj24f06.s1 Homo sapiens cDNA, 3 end /clone=4512; 41522_at Cluster Incl. Z93096:Human DNA sequence from BAC 390B3 on chromosome 22; 2059_s_at M36881 /FEATURE=mRNA /DEFINITION=HUMLCKAA Human lymphocyte-specific pr; 1630_s_at Tyrosine Kinase Syk.

Metagene 365; 34046_at Cluster Incl. Z83844:Human DNA sequence from clone 37E16 on chromosome; 34481_at Cluster Incl. AF030227:untitled /cds=(97,2634) /gb=AF030227 /gi=3282618; 36243_at Cluster Incl. AL050262:Homo sapiens mRNA; cDNA DKFZp547I0610 (from clon; 36709_at Cluster Incl. Y00093:H. sapiens mRNA for leukocyte adhesion glycoprotein; 36753_at Cluster Incl. AF072099:Homo sapiens immunoglobulin-like transcript 3 pr; 36795_at Cluster Incl. J03077:Human co-beta glucosidase (proactivator) mRNA, com; 37105_at Cluster Incl. M16117:Human cathepsin G mRNA, complete cds /cds=(8,775); 37470_at Cluster Incl. AF013249:Homo sapiens leukocyte-associated Ig-like recept; 40390_at Cluster Incl. J05037:Human serine dehydratase m-RNA, complete cds /cds=(; 40745_at Cluster Incl. L13939:Homo sapiens beta adaptin (BAM22) mRNA, complete c; 41059_at Cluster Incl. AF020314:Homo sapiens CMRF-35-H9 mRNA, complete cds /cds=; 31816_at Cluster Incl. X55079:Human lysosomal alpha-glucosidase gene exon 1 /cds; 31826_at Cluster Incl. AB014574:Homo sapiens mRNA for KIAA0674 protein, partial; 31901_at Cluster Incl. AF044253:Homo sapiens potassium channel beta 2 subunit (H; 32116_at Cluster Incl. AB002405:Homo sapiens mRNA for LAK-4p, complete cds /cds=; 32128_at Cluster Incl. Y13710:Homo sapiens mRNA for alternative activated macrop; 32704_at Cluster Incl. D86964:Human mRNA for KIAA0209 gene, partial cds /cds=(0,; 32715_at Cluster Incl. N90862:zb11b06.s1 Homo sapiens cDNA, 3 end /clone=IMAGE-; 33283_at Cluster Incl. AF106941:Homo sapiens beta-arrestin 2 mRNA, complete cds; 33813_at Cluster Incl. AI813532:wj83a09.x1 Homo sapiens cDNA, 3 end /clone=IMAG; 36062_at Cluster Incl. AF062075:Homo sapiens leupaxin mRNA, complete cds /cds=(9; 36493_at Cluster Incl. M33552:Human lymphocyte-specific protein 1 (LSP1) mRNA, c; 36495_at Cluster Incl. U21931:Human fructose-1,6-biphosphatase (FBPI) gene /cds=; 36861_at Cluster Incl. AL049946:Homo sapiens mRNA; cDNA DKFZp564I1922 (from clon; 37591_at Cluster Incl. U94592:Human uncoupling protein homolog (UCPH) mRNA, comp; 38363_at Cluster Incl. W60864:zd27g05.s1 Homo sapiens cDNA, 3 end /clone=IMAGE-; 38671_at Cluster Incl. AB014520:Homo sapiens mRNA for KIAA0620 protein, partial; 39728_at Cluster Incl. J03909:Human gamma-interferon-inducible protein (IP-30) m; 40420_at Cluster Incl. AB015718:Homo sapiens lok mRNA for protein kinase, comple; 41200_at Cluster Incl. Z22555:H. sapiens encoding CLA-1 mRNA /cds=(69,1598) /gb=Z; 32207_at Cluster Incl. M64925:Human palmitoylated erythrocyte membrane protein (; 32773_at Cluster Incl. AA868382:ak41e04.s1 Homo sapiens cDNA, 3 end /clone=IMAG; 32794_g_at Cluster Incl. X00437:Human mRNA for T-cell specific protein /cds=(37,; 33374_at Cluster Incl. L09708:Human complement component 2 (C2) gene allele b /c; 35820_at Cluster Incl. X62078:H. sapiens mRNA for GM2 activator protein /cds=UNKN; 36167_at Cluster Incl. D89052:Homo sapiens mRNA for proton-ATPase-like protein,; 36197_at Cluster Incl. Y08374:H. sapiens gene encoding cartilage GP-39 protein, e; 36207_at Cluster Incl. D67029:Human SEC14L mRNA, complete cds /cds=(303,2450) /g; 36651_at Cluster Incl. X15525:H. sapiens lysosomal acid phosphatase gene (EC 3.1.; 37039_at Cluster Incl. J00194:human hla-dr antigen alpha-chain mma & ivs fragme; 37344_at Cluster Incl. X62744:Human RING6 mRNA for HLA class II alpha chain-like; 37391_at Cluster Incl. X12451:Human mRNA for pro-cathepsin L (major excreted pro; 37395_at Cluster Incl. D49400:Homo sapiens mRNA for vacuolar ATPase, complete cd; 37742_at Cluster Incl. M34423:Human beta-galactosidase (GLB1) mRNA, complete cds; 37759_at Cluster Incl. U51240:Human lysosomal-associated multitransmembrane prot; 38091_at Cluster Incl. Z49107:H. sapiens mRNA for galectin /cds=(87,1058) /gb=Z49; 38095_i_at Cluster Incl. M83664:Human MHC class II lymphocyte antigen (HLA-DP) b; 38391_at Cluster Incl. M94345:Homo sapiens macrophage capping protein mRNA, comp; 38482_at Cluster Incl. AJ011497:Homo sapiens mRNA for Claudin-7 /cds=(334,969) /; 38798_s_at Cluster Incl. AI741833:wg29e04.x1 Homo sapiens cDNA, 3 end /clone=IM; 38833_at Cluster Incl. X00457:Human mRNA for SB classII histocompatibility antig; 40267_s_at Cluster Incl. AI880840:at11d06.x1 Homo sapiens cDNA, 3 end /clone=IM; 1583_at M32315 /FEATURE= /DEFINITION=HUMNFR Human tumor necrosis factor receptor; 1445_at AF014958 /FEATURE= /DEFINITION=AF014958 Homo sapiens chemokine receptor; 1105_s_at M12886 /FEATURE= /DEFINITION=HUMTCBYY Human T-cell receptor active bet; 1106_s_at M12959 /FEATURE= /DEFINITION=HUMTCAXB Human T-cell receptor active alp; 1061_at U00672 /FEATURE= /DEFINITION=U00672 Human interleukin-10 receptor mRNA,; 925_at J03909 /FEATURE= /DEFINITION=HUMIIP Human gamma-interferon-inducible prot; 879_at M30818 /FEATURE=mRNA /DEFINITION=HUMMXB Human interferon-induced cellular; 766_at AB006782 /FEATURE= /DEFINITION=AB006782 Homo sapiens mRNA for galectin-9; 677_s_at J04430 /FEATURE=mRNA /DEFINITION=HUMACP5 Human tartrate-resistant acid; 512_at U22662 /FEATURE= /DEFINITION=HSU22662 Human nuclear orphan receptor LXR-a; 425_at X67325 /FEATURE=cds /DEFINITION=HSP27 H. sapiens p27 mRNA; 239_at M63138 /FEATURE=mRNA /DEFINITION=HUMCATD5 Human cathepsin D (catD) gene,; 189_s_at U09937 /FEATURE=mRNA /DEFINITION=HSUROKR7 Human urokinase-type plasmino.

Metagene 366; 31438_-sat Cluster Incl. Z22971:H. sapiens mRNA for M130 antigen extracellular va; 36255_at Cluster Incl. L77730:Homo sapiens A3 adenosine receptor (ADORA3) gene /; 36708_at Cluster Incl. J04970:Human carboxypeptidase M, 3 end /cds=(0,1319) /gb; 37099_at Cluster Incl. AI806222:wf26e10.x1 Homo sapiens cDNA, 3 end /clone=IMAG; 31820_at Cluster Incl. X16663:Human HS1 gene for heamatopoietic lineage cell spe; 36884_at Cluster Incl. Z22970:H. sapiens mRNA for M130 antigen cytoplasmic varian; 36908_at Cluster Incl. M93221:Human macrophage mannose receptor (MRC1) gene /cds; 37976_at Cluster Incl. AL034397:Human DNA sequence from clone 159A1 on chromosom; 40082_at Cluster Incl. D10040:Homo sapiens mRNA for long-chain acyl-CoA syntheta; 36589_at Cluster Incl. X15414:Human mRNA for aldose reductase (EC 1.1.1.2) /cds=; 36609_at Cluster Incl. D26443:Human mRNA for glutamate transporter, complete cds; 41271_at Cluster Incl. Y18483:Homo sapiens mRNA for SLC7A8 protein /cds=(730,233; 32578_at Cluster Incl. AW005997:wz91c01.x1 Homo sapiens cDNA, 3 end /clone=IMAG; 1795_g_at M92287 /FEATURE= /DEFINITION=HUMCYCD3A Homo sapiens cyclin D3 (CCND3); 581_at M61916 /FEATURE= /DEFINITION=HUMLAM101 Human laminin B1 chain mRNA, compl.

Metagene 367; 32455_s_at Cluster Incl. U88153:Homo sapiens p160 mRNA, partial cds /cds=(0,3755; 32360_s_at Cluster Incl. AF017146:Homo sapiens DNA topoisomerase III beta (TOP3b; 35466_at Cluster Incl. W73046:zd54h09.r1 Homo sapiens cDNA, 5 end /clone=IMAGE-; 38523_f_at Cluster Incl. D49677:Human U2AF1-RS2 mRNA, complete cds /cds=(24,1472; 40705_at Cluster Incl. AF103905:Homo sapiens Rap1 guanine-nucleotide exchange fa; 41426_at Cluster Incl. U38864:Human zinc-finger protein C2H2-150 mRNA, complete; 31873_at Cluster Incl. U52112:ARD1 N-acetyl transferase related protein /cds=(97; 34758_at Cluster Incl. U23028:Human eukaryotic initiation factor 2B-epsilon mRNA; 35225_at Cluster Incl. M91592:Human zinc-finger protein (ZNF76) gene, partial cd; 37955_at Cluster Incl. AB015631:Homo sapiens mRNA for type II membrane protein,; 38682_at Cluster Incl. AF045581:Homo sapiens BRCA1 associated protein 1 (BAP1) m; 39067_at Cluster Incl. M27937:Human male-enhanced antigen mRNA (Mea), complete c; 39729_at Cluster Incl. L19185:Human natural killer cell enhancing factor (NKEFB); 40068_at Cluster Incl. U26648:Homo sapiens syntaxin 5 mRNA, complete cds /cds=(2; 40133_s_at Cluster Incl. W28944:54h12 Homo sapiens cDNA /gb=W28944 /gi=1308955 /; 41734_at Cluster Incl. AB020677:Homo sapiens mRNA for KIAA0870 protein, complete; 33838_at Cluster Incl. M33519:Human HLA-B-associated transcript 3 (BAT3) mRNA, c; 34880_at Cluster Incl. AC002115:Human DNA from overlapping chromosome 19 cosmids; 37319_at Cluster Incl. M35878:Human insulin-like growth factor-binding protein-3; 37667_at Cluster Incl. AF104421:Homo sapiens isolate normal patient 1 uroporphyr; 38376_at Cluster Incl. L46590:Homo sapiens very long chain acyl-CoA dehydrogenas; 38424_at Cluster Incl. AB018290:Homo sapiens mRNA for KIAA0747 protein, partial; 41596_s_at Cluster Incl. U43572:Human alpha-N-acetylglucosaminidase (NAGLU) gene; 1496_at M34668 /FEATURE= /DEFINITION=HUMPTPAAA Human protein tyrosine phosphatas; 1470_at U21090 /FEATURE= /DEFINITION=HSU21090 Human DNA polymerase delta small s; 958_s_at Rna Polymerase II, 14.5 Kda Subunit.

Metagene 368; 34708_at Cluster Incl. D88587:Homo sapiens mRNA for Hakata antigen, complete cds; 32224_at Cluster Incl. AB018312:Homo sapiens mRNA for KIAA0769 protein, complete; 33200_at Cluster Incl. D50550:Human LLGL mRNA, complete cds /cds=(45,3143) /gb=D; 1947_g_at M60614 /FEATURE= /DEFINITION=HUMWITA Human Wilms tumor (WIT-1) associ; 260_at M16447 /FEATURE= /DEFINITION=HUMDHPRA Human dihydropteridine reductase (h.

Metagene 369; 33702_f_at Cluster Incl. L05144:Homo sapiens (clone lamda-hPEC-3) phosphoenolpyr; 39408_at Cluster Incl. Z80345:H. sapiens SCAD gene, 5 UTR exon 1 and 2 (and joine; 39805_at Cluster Incl. AF070598:Homo sapiens clone 24410 ABC transporter mRNA, p.

Metagene 370; 36448_at Cluster Incl. X95677:H. sapiens mRNA for ArgBPIB protein /cds=(134,1033); 36215_at Cluster Incl. M34181:Human testis-specific cAMP-dependent protein kinas; 37162_at Cluster Incl. S72869:H4(D10S170)=putative cytoskeletal protein [human,; 37487_at Cluster Incl. AB029016:Homo sapiens mRNA for KIAA1093 protein, partial; 38208_at Cluster Incl. AB021981:Homo sapiens mRNA for UDP-N-acetylglucosamine tr; 39927_at Cluster Incl. U17032:Human p190-B (p190-B) mRNA, complete cds /cds=(302; 40689_at Cluster Incl. U11037:Human Sel-1 like mRNA, complete cds /cds=(11,298); 32638_s_at Cluster Incl. AI610467:tp42a10.x1 Homo sapiens cDNA, 3 end /clone=IM; 34195_at Cluster Incl. AL121073:DKFZp762B235_r1 Homo sapiens cDNA, 5 end /clone; 34684_at Cluster Incl. L36140:Homo sapiens (clone 1311) DNA helicase (RECQL) mRN; 34716_at Cluster Incl. AF067730:Homo sapiens TLS-associated protein TASR-2 mRNA,; 34717_s_at Cluster Incl. AF047448:Homo sapiens TLS-associated protein TASR mRNA,; 35705_at Cluster Incl. D16815:Homo sapiens mRNA for EAR-1r, complete cds /cds=(3; 33864_at Cluster Incl. X86098:H. sapiens mRNA for BS69 protein /cds=(244,1932) /g; 34886_at Cluster Incl. L02320:Human radixin mRNA, complete cds /cds=(30,1781) /g; 35258_f_at Cluster Incl. AF030234:Homo sapiens splicing factor Sip1 mRNA, comple; 36632_at Cluster Incl. U00957:Human clone KDB1.2 (CAC)n/(GTG)n repeat-containing; 37712_g_at Cluster Incl. S57212:hMEF2C=myocyte enhancer-binding factor 2 [human,; 38100_at Cluster Incl. D87127:Homo sapiens mRNA for translocation protein-1, com; 40555_at Cluster Incl. AL043108:DKFZp434C0823_r1 Homo sapiens cDNA, 5 end /clon; 40928_at Cluster Incl. W26496:30d2 Homo sapiens cDNA /gb=W26496 /gi=1307195 /ug=; 41549_s_at Cluster Incl. AF091077:Homo sapiens clone 558 unknown mRNA, complete; 33103_s_at Cluster Incl. U37122:Human adducin gamma subunit mRNA, complete cds /; 1660_at D83004 /FEATURE= /DEFINITION=D83004 Human epidermoid carcinoma mRNA for; 930_at L07590 /FEATURE= /DEFINITION=HUMPP2A130 Human protein phosphatase 2A 130; 783_at U96113 /FEATURE= /DEFINITION=HSU96113 Homo sapiens Nedd-4-like ubiquitin-; 784_g_at U96113 /FEATURE= /DEFINITION=HSU96113 Homo sapiens Nedd-4-like ubiquiti; 422_s_at X66867 /FEATURE=cds#2 /DEFINITION=HSMAXG H. sapiens max gene.

Metagene 371; 33678_i_at Cluster Incl. X02344:Homo sapiens beta 2 gene /cds=(0,1337) /gb=X0234; 32347_at Cluster Incl. AI831572:wj39c02.x1 Homo sapiens cDNA, 3 end /clone=IMAG; 37425_g_at Cluster Incl. AB029343:Homo sapiens HCR (a-helix coiled-coil rod homo; 37817_at Cluster Incl. AF052087:Homo sapiens clone 23604 mRNA sequence /cds=UNKN; 39973_at Cluster Incl. U47926:Human unknown protein B mRNA, complete cds /cds=(8; 41047_at Cluster Incl. AI885170:wl90e10.x1 Homo sapiens cDNA, 3 end /clone=IMAG; 41610_at Cluster Incl. AB011105:Homo sapiens mRNA for KIAA0533 protein, partial; 41659_at Cluster Incl. U46691:Human putative chromatin structure regulator (SUPT; 41720_r_at Cluster Incl. AF009767:AF009767 Homo sapiens cDNA /clone=C97A-12 /gb=; 34185_at Cluster Incl. W22541:69B4 Homo sapiens cDNA /clone=(not-directional) /g; 34229_s_at Cluster Incl. D84454:Human mRNA for UDP-galactose translocator, compl; 34726_at Cluster Incl. U07139:Human voltage-gated calcium channel beta subunit m; 35627_at Cluster Incl. U40571:Human alpha1-syntrophin (SNT A1) mRNA, complete cd; 37271_at Cluster Incl. L25444:Homo sapiens (TAFII70-alpha) mRNA, complete cds /c; 37904_s_at Cluster Incl. X66436:H. sapiens hsr1 mRNA (partial) /cds=UNKNOWN /gb=X; 38971_r_at Cluster Incl. AJ011896:Homo sapiens mRNA for HIV-1, Nef-associated fa; 39076_s_at Cluster Incl. AI991040:wu36b05.x1 Homo sapiens cDNA, 3 end /clone=IM; 40151_s_at Cluster Incl. Z48054:H. sapiens mRNA for peroxisomal targeting signal; 40470_at Cluster Incl. D10523:Human mRNA for 2-oxoglutarate dehydrogenase, compl; 40867_at Cluster Incl. J02902:Human protein phosphatase 2A regulatory subunit al; 41160_at Cluster Incl. AC005943:Homo sapiens chromosome 19, cosmid R30538 /cds=(; 32166_at Cluster Incl. AB028950:Homo sapiens mRNA for KIAA1027 protein, partial; 32190_at Cluster Incl. AL050118:Homo sapiens mRNA; cDNA DKFZp586C201 (from clone; 33396_at Cluster Incl. U12472:Human glutathione S-transferase (GST phi) gene, co; 33833_at Cluster Incl. J05243:Human nonerythroid alpha-spectrin (SPTAN1) mRNA, c; 33888_at Cluster Incl. X82207:H. sapiens mRNA for beta-centractin (PC3) /cds=(56,; 34318_at Cluster Incl. AJ005896:Homo sapiens mRNA for JM4 protein, complete CDS; 34861_at Cluster Incl. D63997:Homo sapiens mRNA for GCP170, complete cds /cds=(2; 35265_at Cluster Incl. U31501:Human fragile X mental retardation syndrome relate; 35740_at Cluster Incl. AL050138:Homo sapiens mRNA; cDNA DKFZp586M121 (from clone; 36116_at Cluster Incl. AJ000414:Homo sapiens mRNA for Cdc42-interacting protein; 36125_s_at Cluster Incl. L38696:Homo sapiens autoantigen p542 mRNA, complete cds; 36138_at Cluster Incl. X04106:Human mRNA for calcium dependent protease (small s; 36644_at Cluster Incl. D29963:Homo sapiens mRNA for CD151, complete cds /cds=(84; 37329_at Cluster Incl. AF053070:Homo sapiens NADH-ubiquinone dehydrogenase 51 kD; 37410_at Cluster Incl. AJ224358:Homo sapiens surf5a mRNA, clone 1de /cds=(218,64; 37656_at Cluster Incl. D83782:Human mRNA for KIAA0199 gene, partial cds /cds=(0,; 38119_at Cluster Incl. X12496:Human mRNA for erythrocyte membrane sialoglycoprot; 38750_at Cluster Incl. U97669:Homo sapiens Notch3 (NOTCH3) mRNA, complete cds /c; 38809_s_at Cluster Incl. AB011091:Homo sapiens mRNA for KIAA0519 protein, comple; 40597_g_at Cluster Incl. U76366:Human Treacher Collins syndrome (TCOF1) mRNA, co; 40955_at Cluster Incl. U79287:Human clone 23867 mRNA sequence /cds=UNKNOWN /gb=U; 41267_at Cluster Incl. AB028972:Homo sapiens mRNA for KIAA1049 protein, partial; 41526_at Cluster Incl. AF072836:Homo sapiens Sox-like transcriptional factor mRN; 41831_at Cluster Incl. AF077820:Homo sapiens LDL receptor member LR3 mRNA, compl; 32566_at Cluster Incl. AA165701:zo75g08.s1 Homo sapiens cDNA, 3 end /clone=IMAG; 32608_at Cluster Incl. AF000560:Homo sapiens TTF-I interacting peptide 20 mRNA,; 33206_at Cluster Incl. C18655:C18655 Homo sapiens cDNA, 5 end /clone=GEN-565G08; 1590_s_at J00277 /FEATURE=cds /DEFINITION=HUMRASH Human (genomic clones lambda-[; 905_at L76200 /FEATURE= /DEFINITION=HUMGUK1R Human guanylate kinase (GUK1) mRNA,; 692_s_at J02947 /FEATURE=mRNA /DEFINITION=HUMSODEC Human extracellular-superoxid; 564_at M69013 /FEATURE= /DEFINITION=HUMGTPBRPA Human guanine nucleotide-binding; 330_s_at Tubulin, Alpha 1, Isoform 44.

Metagene 372; 36313_at Cluster Incl. M55267:Human EV12 protein gene /cds=(0,698) /gb=M55267 /g; 37148_at Cluster Incl. AF025533:Homo sapiens leucocyte immunoglobulin-like recep; 39248_at Cluster Incl. N74607:za55a01.s1 Homo sapiens cDNA, 3 end /clone=IMAGE-; 40648_at Cluster Incl. U08023:Human cellular proto-oncogene (c-mer) mRNA, comple; 41710_at Cluster Incl. AL079277:Homo sapiens mRNA full length insert cDNA clone; 34215_at Cluster Incl. L03426:Human XE7 mRNA, complete alternate coding regions; 35718_at Cluster Incl. L22342:Human nuclear phosphoprotein mRNA, complete cds /c; 36885_at Cluster Incl. L28824:Homo sapiens protein tyrosine kinase (Syk) mRNA, c; 32227_at Cluster Incl. X17042:Human mRNA for hematopoetic proteoglycan core prot; 34304_s_at Cluster Incl. AL050290:Homo sapiens mRNA; cDNA DKFZp586G1923 (from cl; 35299_at Cluster Incl. AB000409:Homo sapiens mRNA for MNK1, complete cds /cds=(1; 38403_at Cluster Incl. X77196:H. sapiens mRNA for lysosome-associated membrane pr; 40568_at Cluster Incl. L35249:Homo sapiens vacuolar H+-ATPase Mr 56,000 subunit; 41253_s_at Cluster Incl. AI983043:wz30b11.x1 Homo sapiens cDNA, 3 end /clone=IM; 1173_g_at Spermidine/Spermine N1-Acetyltransferase, Alt. Splice 2; 931_at L08177 /FEATURE= /DEFINITION=HUMGPCRB Human EBV induced G-protein coupled; 855_at S78085 /FEATURE= /DEFINITION=S78085 PDCD2=programmed cell death-2 /Rp8 hom; 138_at U66464 /FEATURE= /DEFINITION=HSU66464 Human hematopoietic progenitor kina.

Metagene 373; 32914_f_at Cluster Incl. AC005175:Homo sapiens chromosome 19, cosmid R31449 /cds; 35005_at Cluster Incl. AF051941:Homo sapiens type 6 nucleoside diphosphate kinas; 34207_at Cluster Incl. AB028946:Homo sapiens mRNA for KIAA1023 protein, partial; 35165_at Cluster Incl. AF070582:Homo sapiens clone 24766 mRNA sequence /cds=UNKN; 32825_at Cluster Incl. Y10805:H. sapiens mRNA for arginine methyltransferase, spl; 34853_at Cluster Incl. AB007865:Homo sapiens KIAA0405 mRNA, complete cds /cds=(1; 35741_at Cluster Incl. U85245:Human phosphatidylinositol-4-phosphate 5-kinase ty.

Metagene 374; 33487_at Cluster Incl. D31628:Human gene for 4-hydroxyphenylpyruvic acid dioxyge; 36719_r_at Cluster Incl. L42452:Homo sapiens pyruvate dehydrogenase kinase isoen; 38950_r_at Cluster Incl. AJ005256:Homo sapiens mRNA for MMP-23 /cds=(38,1210) /g; 31855_at Cluster Incl. U61374:Human novel protein with short consensus repeats o; 37599_at Cluster Incl. AF017060:untitled /cds=(298,4314) /gb=AF017060 /gi=234315; 39045_at Cluster Incl. W26655:34c9 Homo sapiens cDNA /gb=W26655 /gi=1307498 /ug=; 35366_at Cluster Incl. M30269:Human nidogen mRNA, complete cds /cds=(90,3833) /g; 38459_g_at Cluster Incl. L39945:Human cytochrome b5 (CYB5) gene /cds=(120,548) /; 39154_at Cluster Incl. AI952982:wp98b06.x1 Homo sapiens cDNA, 3 end /clone=IMAG; 41489_at Cluster Incl. M99435:Human transducin-like enhancer protein (TLE1) mRNA; 1749_at AD000092 /FEATURE=cds#4 /DEFINITION=CH19HHR23 Homo sapiens DNA from chro; 1667_s_at J02871 /FEATURE= /DEFINITION=HUMCP451V Human lung cytochrome P450 (IV; 374_f_at Z84718 /FEATURE=cds#5 /DEFINITION=HS322B1 Human DNA sequence from clone.

Metagene 375; 35868_at Cluster Incl. M91211:Human receptor for advanced glycosylation end prod; 41356_at Cluster Incl. W27619:35c7 Homo sapiens cDNA /gb=W27619 /gi=1307567 /ug=; 33196_at Cluster Incl. U08191:Human R kappa B mRNA, complete cds /cds=(2220,5216; 1907_at L14812 /FEATURE= /DEFINITION=HUMP107B Human retinoblastoma related prote.

Metagene 376; 31993_f_at Cluster Incl. U80764:Human EST clone 122887 mariner transposon Hsmar1; 32401_at Cluster Incl. U78110:Human prepro-neurturin mRNA, complete cds /cds=(0,; 33493_at Cluster Incl. AF048849:Homo sapiens erythroid differentiation and denuc; 33521_at Cluster Incl. M63962:Human gastric H,K-ATPase catalytic subunit gene, c; 34453_at Cluster Incl. H19081:yn51e03.r1 Homo sapiens cDNA, 5 end /clone=IMAGE-; 39684_at Cluster Incl. U37707:Human dlg3 mRNA, complete cds /cds=(336,2093) /gb=; 40643_at Cluster Incl. M34480:Human platelet glycoprotein IIb (GPIIb) mRNA, comp; 40729_s_at Cluster Incl. Y14768:Homo sapiens DNA, cosmid clones TN62 and TN82 /c; 31819_at Cluster Incl. AI796281:wh49a09.x1 Homo sapiens cDNA, 3 end /clone=IMAG; 35630_at Cluster Incl. X87342:H. sapiens mRNA for human giant larvae homolog /cds; 36451_at Cluster Incl. AI743299:wg91b04.x1 Homo sapiens cDNA, 3 end /clone=IMAG; 39335_at Cluster Incl. AI074025:oy66g12.x1 Homo sapiens cDNA, 3 end /clone=IMAG; 32228_at Cluster Incl. AB020706:Homo sapiens mRNA for KIAA0899 protein, partial; 40534_at Cluster Incl. AA843737:ak01e07.s1 Homo sapiens cDNA, 3 end /clone=IMAG; 1296_at D83542 /FEATURE= /DEFINITION=HUMC15A Homo sapiens mRNA for cadherin-15,; 385_at X71874 /FEATURE=cds#2 /DEFINITION=HSPROSCHY H. sapiens genes for proteasom.

Metagene 377; 32441_at Cluster Incl. X52142:Human mRNA for CTP synthetase (EC 6.3.4.2) /cds=(7; 32371_at Cluster Incl. AB011099:Homo sapiens mRNA for KIAA0527 protein, partial; 38577_at Cluster Incl. U92981:Homo sapiens clone DT1P1B6 mRNA, CAG repeat region; 39932_at Cluster Incl. AI655015:wb66a10.x1 Homo sapiens cDNA, 3 end /clone=IMAG; 40318_at Cluster Incl. AI810807:tu26a01.x1 Homo sapiens cDNA, 3 end /clone=IMAG; 41031_at Cluster Incl. AB000114:Homo sapiens mRNA for osteomodulin, complete cds; 41464_at Cluster Incl. AB002337:Human mRNA for KIAA0339 gene, complete cds /cds=; 32076_at Cluster Incl. D83407:ZAKI-4 mRNA in human skin fibroblast, complete cds; 35223_at Cluster Incl. AB023234:Homo sapiens mRNA for KIAA1017 protein, complete; 35649_at Cluster Incl. U80055:untitled /cds=(248,850) /gb=U80055 /gi=2138110 /ug; 36092_at Cluster Incl. AL080213:Homo sapiens mRNA; cDNA DKFZp586I1823 (from clon; 39425_at Cluster Incl. X91247:H. sapiens mRNA for thioredoxin reductase /cds=(439; 40045_g_at Cluster Incl. AF009425:Homo sapiens clone 22 mRNA, alternative splici; 33878_at Cluster Incl. W27472:31d4 Homo sapiens cDNA /gb=W27472 /gi=1307276 /ug=; 33905_at Cluster Incl. AF072242:Homo sapiens methyl-CpG binding protein MBD2 (MB; 35332_at Cluster Incl. X04714:Human mRNA for apolipoprotein B-100 (apoB-100) /cd; 38028_at Cluster Incl. AL050152:Homo sapiens mRNA; cDNA DKFZp586K1220 (from clon; 38469_at Cluster Incl. M35252:Human CO-029 /cds=(137,850) /gb=M35252 /gi=180925; 38748_at Cluster Incl. U76421:Human dsRNA adenosine deaminase DRADA2b (DRADA2b); 40230_at Cluster Incl. U91903:Human Fritz mRNA, complete cds /cds=(69,1046) /gb=; 40530_at Cluster Incl. AL080130:Homo sapiens mRNA; cDNA DKFZp434E033 (from clone; 1736_at M62402 /FEATURE= /DEFINITION=HUMIGFBP4 Human insulin-like growth factor; 1685_at X82554 /FEATURE=mRNA /DEFINITION=HSSPHAR H. sapiens SPHAR gene for cyclin; 1467_at U12535 /FEATURE= /DEFINITION=HSU12535 Human epidermal growth factor rece.

Metagene 378; 31530_at Cluster Incl. U89344:Human acetyl-CoA carboxylase (ACC2) mRNA, complete; 32275_at Cluster Incl. X04470:Human mRNA for antileukoprotease (ALP) from cervix; 34922_at Cluster Incl. AF047826:Homo sapiens cadherin-7 (CDH7) mRNA, partial cds; 41622_r_at Cluster Incl. AA868898:ak55b08.s1 Homo sapiens cDNA, 3 end /clone=IM; 34183_at Cluster Incl. AL080169:Homo sapiens mRNA; cDNA DKFZp434C171 (from clone; 36018_at Cluster Incl. AJ00183:Homo sapiens mRNA for Sox10 protein /cds=(120,15; 35817_at Cluster Incl. M13577:Human myelin basic protein (MBP) mRNA, complete cd; 38408_at Cluster Incl. L10373:Human (clone CCG-B7) mRNA sequence /cds=UNKNOWN /g; 38747_at Cluster Incl. M81945:Human CD34 gene, promoter and /cds=(258,1415) /gb=; 1252_at M73547 /FEATURE= /DEFINITION=HUMPOLLA Human polyposis locus (DP1 gene) m.

Metagene 379; 39236_-sat Cluster Incl. AL050372:Homo sapiens mRNA; cDNA DKFZp434A091 (from clo; 32073_at Cluster Incl. AB014577:Homo sapiens mRNA for KIAA0677 protein, complete; 33287_at Cluster Incl. AA533071:nj19e03.s1 Homo sapiens cDNA, 3 end /clone=IMAG; 36876_at Cluster Incl. M55150:Human fumarylacetoacetate hydrolase mRNA, complete; 32221_at Cluster Incl. AL050361:Homo sapiens mRNA; cDNA DKFZp564H0223 (from clon; 34775_at Cluster Incl. AF065388:Homo sapiens tetraspan NET-1 mRNA, complete cds; 36664_at Cluster Incl. M60091:Homo sapiens galactose-1-phosphate uridyl transfer; 36985_at Cluster Incl. X17025:Human homolog of yeast IPP isomerase /cds=(50,736); 37315_f_at Cluster Incl. AI057607:oy31e07.x1 Homo sapiens cDNA, 3 end /clone=IM; 38794_at Cluster Incl. X53390:Human mRNA for upstream binding factor (hUBF) /cds; 39516_at Cluster Incl. AI827793:wf33b11.x1 Homo sapiens cDNA, 3 end /clone=IMAG; 41540_at Cluster Incl. Z50749:H. sapiens sds22-like mRNA /cds=(15,1097) /gb=Z5074.

Metagene 380; 36260_at Cluster Incl. AB002448:Homo sapiens mRNA from chromosome 5q21-22, clone; 39941_at Cluster Incl. Z75311:H. sapiens mRNA for RAD50 /cds=(42,3998) /gb=Z75311; 32679_at Cluster Incl. D13634:Human mRNA for KIAA0009 gene, complete cds /cds=(1; 34259_at Cluster Incl. AB014564:Homo sapiens mRNA for KIAA0664 protein, partial; 38361_g_at Cluster Incl. AI688812:wd41c03.x1 Homo sapiens cDNA, 3 end /clone=IM; 38620_at Cluster Incl. AA905543:oj86h04.s1 Homo sapiens cDNA, 3 end /clone=IMAG; 33918_s_at Cluster Incl. AJ223349:Homo sapiens mRNA for HIRIP3 protein, clone pH; 40228_at Cluster Incl. Y11392:Homo sapiens mRNA; candidate gene for APECED /cds=; 41806_at Cluster Incl. J04513:Human basic fibroblast growth factor (bFGF) 22.5 k.

Metagene 381; 34591_at Cluster Incl. S79854:type 3 iodothyronine deiodinase=selenoenzyme [huma; 36410_f_at Cluster Incl. U12431:Human ELAV-like neuronal protein 1 (hel-N1) mRNA; 36038_r_at Cluster Incl. J05500:Human beta-spectrin (SPTB) mRNA, complete cds /c; 32152_at Cluster Incl. X16609:Human mRNA for ankyrin (variant 2.1) /cds=(84,5729; 39454_f_at Cluster Incl. AJ002607:Homo sapiens HOX11L1 gene, exon 1 and joined C; 1777_at L36463 /FEATURE= /DEFINITION=HUMA Homo sapiens ras interactor (RIN1) mRN.

Metagene 382; 38488_s_at Cluster Incl. AF031167:Homo sapiens interleukin 15 precursor (IL-15); 39592_r_at Cluster Incl. Z36531:H. sapiens mRNA for fibrinogen-like protein (pT49; 41436_at Cluster Incl. AJ224901:Homo sapiens mRNA for ZNF198 protein /cds=(184,4; 41634_at Cluster Incl. D87445:Human mRNA for KIAA0256 gene, complete cds /cds=(1; 41683_i_at Cluster Incl. U50708:Human branched chain alpha-ketoacid dehydrogenas; 41713_at Cluster Incl. U09848:Human zinc finger protein (ZNF139) mRNA, partial c; 32628_at Cluster Incl. D28118:Human mRNA for DB1, complete cds /cds=(41,1591) /g; 32664_at Cluster Incl. D37931:Human mRNA for RNase 4, complete cds /cds=(27,470); 32667_at Cluster Incl. M58526:Human alpha-5 collagen type IV (COL4A5) mRNA, 3 e; 32668_at Cluster Incl. AL080076:Homo sapiens mRNA; cDNA DKFZp564C0362 (from clon; 32740_at Cluster Incl. AB023158:Homo sapiens mRNA for KIAA0941 protein, complete; 33700_at Cluster Incl. AF039843:Homo sapiens Sprouty 2 (SPRY2) mRNA, complete cd; 34739_at Cluster Incl. W26023:18c3 Homo sapiens cDNA /gb=W26023 /gi=1306308 /ug=; 34760_at Cluster Incl. D14664:Human mRNA for KIAA0022 gene, complete cds /cds=(1; 34767_at Cluster Incl. AI670788:tz10c02.x1 Homo sapiens cDNA, 3 end /clone=IMAG; 35195_at Cluster Incl. Y11651:H. sapiens mRNA for phosphate cyclase /cds=(170,127; 35209_at Cluster Incl. AB018309:Homo sapiens mRNA for KIAA0766 protein, complete; 36074_at Cluster Incl. U12897:Homo sapiens IPW mRNA sequence /cds=UNKNOWN /gb=U1; 36509_at Cluster Incl. AL049998:Homo sapiens mRNA; cDNA DKFZp564L222 (from clone; 36896_s_at Cluster Incl. AF044288:Homo sapiens basic-helix-loop-helix-PAS orphan; 38256_s_at Cluster Incl. W21827:57E11 Homo sapiens cDNA /clone=(not-directional); 38659_at Cluster Incl. AB020669:Homo sapiens mRNA for KIAA0862 protein, complete; 39021_at Cluster Incl. AB020684:Homo sapiens mRNA for KIAA0877 protein, partial; 39780_at Cluster Incl. M29551:Human calcineurin A2 mRNA, complete cds /cds=(116,; 39793_at Cluster Incl. AF029786:Homo sapiens GBAS (GBAS) mRNA, complete cds /cds; 40046_r_at Cluster Incl. AF009426:Homo sapiens clone 22 mRNA, alternative splice; 40431_at Cluster Incl. AB007891:Homo sapiens KIAA0431 mRNA, partial cds /cds=(96; 40786_at Cluster Incl. U37352:Human protein phosphatase 2A Balpha1 regulatory su; 41780_at Cluster Incl. U22816:Human LAR-interacting protein, 1b mRNA, complete cd; 32781_f_at Cluster Incl. AA058762:zk65d06.r1 Homo sapiens cDNA, 5 end /clone=IM; 32851_at Cluster Incl. AF036956:Homo sapiens neuroblastoma apoptosis-related RNA; 33440_at Cluster Incl. U19969:Human two-handed zinc finger protein ZEB mRNA, par; 33831_at Cluster Incl. U47741:Human CREB-binding protein (CBP) mRNA, complete cd; 34355_at Cluster Incl. AJ132917:Homo sapiens mRNA for methyl-CpG-binding protein; 34383_at Cluster Incl. AB014458:Homo sapiens hUBP mRNA for ubiquitin specific pr; 34842_at Cluster Incl. U41303:Human small nuclear ribonuleoprotein particle N(S; 35767_at Cluster Incl. AI565760:tn20b01.x1 Homo sapiens cDNA, 3 end /clone=IMAG; 36571_at Cluster Incl. X68060:H. sapiens topIIb mRNA for topoisomerase IIb /cds=(; 36580_at Cluster Incl. AL050139:Homo sapiens mRNA; cDNA DKFZp586M141 (from clone; 36948_at Cluster Incl. AL109701:Homo sapiens mRNA full length insert cDNA clone; 36953_at Cluster Incl. U44378:Human homozygous deletion target in pancreatic car; 37038_at Cluster Incl. X83467:H. sapiens PXMP1 gene, exon 1 (and joined CDS) /cds; 37334_at Cluster Incl. U23803:Human heterogeneous ribonucleoprotein A0 mRNA, com; 37697_s_at Cluster Incl. L08666:Homo sapiens porin (por) mRNA, complete cds and; 37727_i_at Cluster Incl. X78669:H. sapiens ERC-55 mRNA /cds=(66,1019) /gb=X78669; 39091_at Cluster Incl. AF070523:Homo sapiens JWA protein mRNA, complete cds /cds; 39140_at Cluster Incl. AL079292:Homo sapiens m-RNA full length insert cDNA clone; 40601_at Cluster Incl. AI057115:oz23g10.x1 Homo sapiens cDNA, 3 end /clone=IMAG; 40631_at Cluster Incl. D38305:Human mRNA for Tob, complete cds /cds=(43,1080) /g; 40893_at Cluster Incl. AF058953:Homo sapiens ATP-specific succinyl-CoA synthetas; 41808_at Cluster Incl. AF052102:Homo sapiens clone 23926 mRNA sequence /cds=UNKN; 2086_s_at D17517 /FEATURE= /DEFINITION=HUMSKY Human sky mRNA for Sky, complete c; 1307_at D14533 /FEATURE= /DEFINITION=HUMXPAC Human mRNA for XPAC protein; 1192_at AB003103 /FEATURE= /DEFINITION=AB003103 Homo sapiens mRNA for 26S protea; 779_at D21337 /FEATURE= /DEFINITION=HUMCO Human mRNA for collagen.

Metagene 383; 31909_at Cluster Incl. AB018297:Homo sapiens mRNA for KIAA0754 protein, partial; 31790_at Cluster Incl. AL049801:Novel human gene mapping to chomosome 13, simila; 32727_at Cluster Incl. AF037062:Homo sapiens retinol dehydrogenase gene, complet; 33362_at Cluster Incl. AF094521:Homo sapiens MSE55-related protein (UB1) mRNA, c.

Metagene 384; 31655_at Cluster Incl. AL031737:Human DNA sequence from clone 8B22 on chromosome; 31692_at Cluster Incl. M59830:Human MHC class III HSP70-2 gene (HLA), complete c; 36785_at Cluster Incl. Z23090:H. sapiens mRNA for 28 kDa heat shock protein /cds=; 37158_at Cluster Incl. AF035306:Homo sapiens clone 23771 mRNA sequence /cds=UNKN; 41378_at Cluster Incl. AF010236:Homo sapiens mRNA from chromosome 5q31-33 region; 32108_at Cluster Incl. M76231:Human sepiapterin reductase mRNA, complete cds /cd; 33816_at Cluster Incl. AF020267:Homo sapiens myosin-IXb splice variant (Myo9b) m; 34246_at Cluster Incl. AA418437:zv92d11.r1 Homo sapiens cDNA, 5 end /clone=IMAG; 35191_at Cluster Incl. AB002373:Human mRNA for KIAA0375 gene, complete cds /cds=; 37579_at Cluster Incl. L47738:Homo sapiens inducible protein mRNA, complete cds; 37637_at Cluster Incl. U27655:Human RGP3 mRNA, complete cds /cds=(287,1846) /gb=; 38700_at Cluster Incl. M33146:Human cysteine-rich peptide m-RNA, complete cds /cd; 39066_at Cluster Incl. L38486:Human microfibril-associated glycoprotein 4 (MFAP4; 39709_at Cluster Incl. U67171:Human selenoprotein W (selW) mRNA, complete cds /c; 40120_at Cluster Incl. X90999:H. sapiens mRNA for Glyoxalase II /cds=(36,818) /gb; 40427_at Cluster Incl. AA149486:zl27g01.r1 Homo sapiens cDNA, 5 end /clone=IMAG; 41753_at Cluster Incl. U48734:Human non-muscle alpha-actinin mRNA, complete cds; 32164_at Cluster Incl. S79639:EXT1=putative tumour suppressor/hereditary multipl; 32226_at Cluster Incl. M64571:Human microtubule-associated protein 4 mRNA, compl; 32260_at Cluster Incl. X86809:H. sapiens mRNA for major astrocytic phosphoprotein; 32749_s_at Cluster Incl. AL050396:Homo sapiens mRNA; cDNA DKFZp586K1720 (from cl; 32750_r_at Cluster Incl. X53416:Human mRNA for actin-binding protein (filamin) (; 33850_at Cluster Incl. W28892:53c11 Homo sapiens cDNA /gb=W28892 /gi=1308840 /ug; 33882_at Cluster Incl. AB020664:Homo sapiens mRNA for KIAA0857 protein, partial; 33903_at Cluster Incl. AB007144:Homo sapiens mRNA for ZIP-kinase, complete cds /; 35365_at Cluster Incl. U40282:Homo sapiens integrin-linked kinase (ILK) mRNA, co; 36937_s_at Cluster Incl. U90878:Homo sapiens carboxyl terminal LIM domain protei; 39115_at Cluster Incl. AL050275:Homo sapiens mRNA; cDNA DKFZp566D213 (from clone; 40900_at Cluster Incl. AI382123:te30a09.x1 Homo sapiens cDNA, 3 end /clone=IMAG; 41288_at Cluster Incl. AL036744:DKFZp564I1663_r1 Homo sapiens cDNA, 5 end /clon; 41528_at Cluster Incl. W72239:zd62h08.s1 Homo sapiens cDNA, 3 end /clone=IMAGE-; 32522_f_at Cluster Incl. M20469:Human brain-type clathrin light-chain b mRNA, co; 1561_at U27193 /FEATURE= /DEFINITION=HSU27193 Human protein-tyrosine phosphatase; 1176_at Integrin, Beta 3 Subunit; 1104_s_at M11717 /FEATURE=mRNA /DEFINITION=HUMHSP70D Human heat shock protein (h; 897_at L33243 /FEATURE=mRNA /DEFINITION=HUMPKD1A Homo sapiens polycystic kidney; 635_s_at L42374 /FEATURE=mRNA /DEFINITION=HUMPP2ABA Homo sapiens protein phospha; 243_g_at M64571 /FEATURE=mRNA /DEFINITION=HUMMAP4 Human microtubule-associated p; 222_at S79639 /FEATURE= /DEFINITION=S79639 EXT1=putative tumour suppressor/hered.

Metagene 385; 31417_at Cluster Incl. AF041240:Homo sapiens prepro-orexin mRNA, complete cds /c; 34600_s_at Cluster Incl. U54644:Human tub homolog mRNA, complete cds /cds=(152,1; 36407_at Cluster Incl. AL050220:Homo sapiens mRNA; cDNA DKFZp586J1923 (from clon; 36408_at Cluster Incl. AB011082:Homo sapiens ORCTL4 mRNA for organic-cation tran; 32913_i_at Cluster Incl. AC005175:Homo sapiens chromosome 19, cosmid R31449 /cds; 35960_at Cluster Incl. AF031416:Homo sapiens IkB kinase beta subunit mRNA, compl; 39570_at Cluster Incl. W22289:65G5 Homo sapiens cDNA /clone=(not-directional) /g; 39598_at Cluster Incl. X04325:Human liver mRNA for gap junction protein /cds=(62; 41657_at Cluster Incl. AF035625:Homo sapiens serine threonine kinase 11 (STK11); 40898_at Cluster Incl. U46751:Human phosphotyrosine independent ligand p62 for t.

Metagene 386; 33030_at Cluster Incl. M60747:Human histone H1 (H1F3) gene, complete cds /cds=(0; 34541_at Cluster Incl. L02867:Homo sapiens 62 kDa paraneoplastic antigen mRNA, 3; 38486_at Cluster Incl. J04760:Human slow-twitch skeletal troponin I (TNN1) mRNA,; 1332_f_at V00520 /FEATURE=mRNA /DEFINITION=HSGROW2 Human germ line gene for grow; 1033_g_at U11872 /FEATURE= /DEFINITION=HSU11872 Human interleukin-8 receptor typ; 643_at L76571 /FEATURE=cds /DEFINITION=HUMSHP Homo sapiens nuclear hormone recep.

Metagene 387; 34595_at Cluster Incl. AF105424:Homo sapiens brush border myosin I (BBMI) mRNA,; 32286_at Cluster Incl. AF012131:Homo sapiens brachyury variant B (TBX1) mRNA, co; 37860_at Cluster Incl. AL049942:Homo sapiens mRNA; cDNA DKFZp564F1422 (from clon; 32097_at Cluster Incl. AB007862:Homo sapiens KIAA0402 mRNA, partial cds /cds=(0,; 35240_at Cluster Incl. W28983:54f11 Homo sapiens cDNA /gb=W28983 /gi=1308931 /ug; 36035_at Cluster Incl. AB002135:Homo sapiens mRNA for glycosylphosphatidylinosit; 33868_at Cluster Incl. Z93241:dJ222E13.3.2 (PUTATIVE partial isoform 2) /cds=(0,; 40222_s_at Cluster Incl. AI677689:wd33c06.x1 Homo sapiens cDNA, 3 end /clone=IM.

Metagene 388; 35769_at Cluster Incl. AJ011001:Homo sapiens mRNA for TM7XN1 protein /cds=(316,2; 619_s_at M27394 /FEATURE=cds /DEFINITION=HUMB1LYM Human B-lymphocyte cell-surfac; 459_s_at U68485 /FEATURE= /DEFINITION=HSU68485 Homo sapiens bridging integrator.

Metagene 389; 33041_at Cluster Incl. U03493:Human connexin45 gene, complete cds /cds=(0,1190); 41861_at Cluster Incl. AL050019:Homo sapiens mRNA; cDNA DKFZp564C186 (from clone; 1868_g_at AF005775 /FEATURE= /DEFINITION=AF005775 Homo sapiens caspase-like apop; 698_f_at Zinc Finger Protein, Kruppel-Like.

Metagene 390; 31557_at Cluster Incl. M17733:Human thymosin beta-4 mRNA, complete cds /cds=(77,; 33089_s_at Cluster Incl. U92896:Human LERK-6 (EPLG6) gene /cds=(0,641) /gb=U9289; 35523_at Cluster Incl. AF150241:AF150241 Homo sapiens cDNA /clone=CBFAZB10 /gb=A; 38160_at Cluster Incl. AF011333:Homo sapiens DEC-205 mRNA, complete cds /cds=(53; 40311_at Cluster Incl. AF053356:Homo sapiens chromosome 7q22 sequence /cds=(27,2.

Metagene 391; 35039_at Cluster Incl. D87466:Human mRNA for KIAA0276 gene, partial cds /cds=(0,; 38204_at Cluster Incl. AB007866:Homo sapiens KIAA0406 mRNA, complete cds /cds=(1; 38892_at Cluster Incl. D87077:Human mRNA for KIAA0240 gene, partial cds /cds=(0,; 39646_at Cluster Incl. S60415:myasthenic syndrome antigen B [human, fetal brain,; 39989_at Cluster Incl. X90530:H. sapiens mRNA for ragB protein /cds=(442,1566) /g; 40732_at Cluster Incl. D83243:Human NPAT mRNA, complete cds /cds=(66,4349) /gb=D; 33760_at Cluster Incl. AB017546:Homo sapiens Pex14 mRNA for peroxisomal membrane; 34198_at Cluster Incl. U12128:Human protein tyrosine phosphatase 1E (PTP1E) mRNA; 34675_at Cluster Incl. AL080210:Homo sapiens mRNA; cDNA DKFZp586G0623 (from clon; 35167_at Cluster Incl. AB007893:Homo sapiens KIAA0433 mRNA, partial cds /cds=(50; 35232_f_at Cluster Incl. AI056696:oz26h05.x1 Homo sapiens cDNA, 3 end /clone=IM; 35645_at Cluster Incl. AL050148:Homo sapiens mRNA; cDNA DKFZp586G1520 (from clon; 39036_g_at Cluster Incl. AF006010:Human progestin induced protein (DD5) mRNA, co; 39047_at Cluster Incl. AB020880:Homo sapiens mRNA for squamous cell carcinoma an; 39346_at Cluster Incl. M88108:Human p62 mRNA, complete cds /cds=(106,1437) /gb=M; 39390_at Cluster Incl. AF052123:Homo sapiens clone 23770 mRNA sequence /cds=UNKN; 39443_s_at Cluster Incl. M19961:Human cytochrome c oxidase subunit Vb (coxyb) mR; 39731_at Cluster Incl. Z23064:H. sapiens mRNA gene for hnRNP G protein /cds=(11,1; 39782_at Cluster Incl. X95592:H. sapiens mRNA for CID protein /cds=(117,542) /gb=; 40763_at Cluster Incl. U85707:Human leukemogenic homolog protein (MEIS1) mRNA, c; 40781_at Cluster Incl. U79271:Human clones 23920 and 23921 mRNA sequence /cds=UN; 40826_at Cluster Incl. M80359:Human protein p78 mRNA, complete cds /cds=(171,231; 41219_at Cluster Incl. AL050376:Homo sapiens mRNA; cDNA DKFZp586J101 (from clone; 32171_at Cluster Incl. AL080102:Homo sapiens mRNA; cDNA DKFZp564N1916 (from clon; 32172_at Cluster Incl. AL096858:Novel human gene mapping to chomosome 1 /cds=(33; 32792_at Cluster Incl. AL031432:Human DNA sequence from clone 465N24 on chromoso; 33847_s_at Cluster Incl. AI304854:qo19f03.x1 Homo sapiens cDNA, 3 end /clone=IM; 34385_at Cluster Incl. U57877:Human integral membrane protein CII-3 mRNA, nuclea; 35755_at Cluster Incl. U51336:Human inositol 1,3,4-trisphosphate 5/6-kinase mRNA; 36153_at Cluster Incl. L13848:Human RNA helicase A mRNA, complete cds /cds=(80,3; 36164_at Cluster Incl. U82328:Homo sapiens pyruvate dehydrogenase complex protei; 36974_at Cluster Incl. D88378:Homo sapiens mRNA for proteasome inhibitor hPI31 s; 37034_at Cluster Incl. U73477:Human acidic nuclear phosphoprotein pp32 mRNA, com; 38481_at Cluster Incl. M63488:Human replication protein A 70 kDa subunit mRNA com; 39864_at Cluster Incl. D78134:Homo sapiens mRNA for CIRP, complete cds /cds=(80,; 40211_at Cluster Incl. X12671:Human gene for heterogeneous nuclear ribonucleopro; 40576_f_at Cluster Incl. D89678:Homo sapiens mRNA for A+U-rich element RNA bindi; 41490_at Cluster Incl. Y00971:Human mRNA for phosphoriobosyl pyrophosphate synth; 41544_at Cluster Incl. AF059617:Homo sapiens serum-inducible kinase mRNA, comple; 32609_at Cluster Incl. AI885852:wl62d08.x1 Homo sapiens cDNA, 3 end /clone=IMAG; 1488_at L77886 /FEATURE= /DEFINITION=HUMPTPC Human protein tyrosine phosphatase.

Metagene 392; 31460_f_at Cluster Incl. AF043586:Homo sapiens clone CPRF1-T2 immunoglobulin lam; 33627_at Cluster Incl. U57843:Human phosphatidylinositol 3-kinase delta catalyti; 35601_at Cluster Incl. L00022:Human Ig active epsilon1 5 UT, V-D-J region subgro; 41054_at Cluster Incl. AB006628:Homo sapiens mRNA for KIAA0290 gene, partial cds.

Metagene 393; 41676_at Cluster Incl. U79248:Human clone 23826 mRNA sequence /cds=UNKNOWN /gb=U; 32045_at Cluster Incl. AB002331:Human mRNA for KLAA0333 gene, partial cds /cds=(; 40138_at Cluster Incl. U70735:Homo sapiens 34 kDa Mov34 homolog mRNA, complete c; 32241_at Cluster Incl. AL050265:Homo sapiens mRNA; cDNA DKFZp564O1716 (from clon; 33854_at Cluster Incl. AA877795:nr10g08.s1 Homo sapiens cDNA, 3 end /clone=IMAG; 39196_i_at Cluster Incl. W25875:14f10 Homo sapiens cDNA /gb=W25875 /gi=1306016 /.

Metagene 394; 36734_at Cluster Incl. M21302:Human small proline rich protein (sprII) mRNA, clo; 37424_at Cluster Incl. AB029343:Homo sapiens HCR (a-helix coiled-coil rod homolo; 40711_at Cluster Incl. AL049340:Homo sapiens mRNA; cDNA DKFZp564P056 (from clone; 33321_r_at Cluster Incl. M33494:Homo sapiens tryptase-I gene, complete cds /cds=; 35650_at Cluster Incl. AB002354:Human mRNA for KIAA0356 gene, complete cds /cds=; 38995_at Cluster Incl. AF000959:Homo sapiens transmembrane protein mRNA, complet; 33895_at Cluster Incl. AL050373:Homo sapiens mRNA; cDNA DKFZp586F1318 (from clon; 38104_at Cluster Incl. U78302:Human 2,4-dienoyl-CoA reductase gene /cds=(73,1080; 1128_s_at L09230 /FEATURE= /DEFINITION=HUMCCCKR1A Human C-C chemokine receptor t; 802_at X84002 /FEATURE=cds /DEFINITION=HSTAFII20 H. sapiens TAFII20 mRNA for tran.

Metagene 395; 34976_at Cluster Incl. M60052:Human histidine-rich calcium binding protein (HRC); 41110_at Cluster Incl. X81882:H. sapiens mRNA for for vasopressin activated calci; 35203_at Cluster Incl. AB002381:Human mRNA for KIAA0383 gene, partial cds /cds=(; 37995_s_at Cluster Incl. M67468:Human Fragile X mental retardation 1 FMR-1 gene,; 32552_at Cluster Incl. X00129:Human mRNA for retinol binding protein (RBP) /cds=; 1453_at U68018 /FEATURE= /DEFINITION=HSU68018 Human mad protein homolog (hMAD-2).

Metagene 396; 39574_at Cluster Incl. AB021292:Homo sapiens mRNA for semaphorin W, splicing var; 40309_at Cluster Incl. X66839:H. sapiens MaTu MN mRNA for p54/58N protein /cds=(4; 40782_at Cluster Incl. AF061741:Homo sapiens retinal short-chain dehydrogenase/r; 32177_s_at Cluster Incl. AC004084:Homo sapiens BAC clone RG158017 from 7q22-q31.; 34407_at Cluster Incl. U77594:Human tazarotene-induced gene 2 (TIG2) mRNA, compl; 2051_at M31767 /FEATURE=mRNA /DEFINITION=HUMDNAMET Human 06-methylguanine-DNA me; 1057_at M97815 /FEATURE=expanded cds /DEFINITION=HUMCRABP02 Human retinoic acid-.

Metagene 397; 37122_at Cluster Incl. AB005293:Homo sapiens mRNA for perilipin, complete cds /c; 37513_at Cluster Incl. Y13647:Homo sapiens mRNA for stearoyl-CoA desaturase /cds; 40657_r_at Cluster Incl. H15814:yl28b07.s1 Homo sapiens cDNA, 3 end /clone=IMAG; 40658_r_at Cluster Incl. D45371:Human apM1 mRNA for GS3109 (novel adipose specif; 33703_f_at Cluster Incl. L12760:Human phosphoenolpyruvate carboxykinase (PCK1) g; 38320_s_at Cluster Incl. L11706:Human hormone-sensitive lipase (LIPE) gene, comp; 33902_at Cluster Incl. L34041:Homo sapiens L-glycerol-3-phosphate-NAD oxidoreduc; 38429_at Cluster Incl. U29344:Human breast carcinoma fatty acid synthase mRNA, c; 40282_s_at Cluster Incl. M84526:Human adipsin/complement factor D mRNA, complete.

Metagene 398; 31584_at Cluster Incl. X16064:Human mRNA for translationally controlled tumor pr; 34030_at Cluster Incl. U82468:Human tubby related protein 1 (TULP1) mRNA, comple; 36306_at Cluster Incl. AF033347:Homo sapiens potassium channel homolog (KCNQ3) m; 33457_at Cluster Incl. AB029028:Homo sapiens mRNA for KIAA1105 protein, partial; 37009_at Cluster Incl. AL035079:dJ53C18.1 (Catalase) /cds=(74,1657) /gb=AL035079; 1815_g_at D50683 /FEATURE= /DEFINITION=D50683 Homo sapiens mRNA for TGF-betaIIR.

Metagene 399; 36736_f_at Cluster Incl. Y10275:H. sapiens mRNA for L-3-phosphoserine phosphatase; 37208_at Cluster Incl. AJ001612:Homo sapiens mRNA for L-3-phosphoserine-phosphat; 37209_g_at Cluster Incl. AJ001612:Homo sapiens mRNA for L-3-phosphoserine-phosph; 40272_at Cluster Incl. D78012:Homo sapiens mRNA for dihydropyrimidinase related; 1572_s_at M10051 /FEATURE= /DEFINITION=HUMINSR Human insulin receptor mRNA, comp; 1130_at L11284 /FEATURE= /DEFINITION=HUMMEK1NF Homo sapiens ERK activator kinase.

Metagene 400; 39296_at Cluster Incl. W28319:45c1 Homo sapiens cDNA /gb=W28319 /gi=1308267 /ug=; 41057_at Cluster Incl. AI760162:wg58e09.x1 Homo sapiens cDNA, 3 end /clone=IMAG; 41371_at Cluster Incl. Y07595:H. sapiens mRNA for 52 kD subunit of transcription; 35149_at Cluster Incl. AI865431:wk11h09.x1 Homo sapiens cDNA, 3 end /clone=IMAG; 36867_at Cluster Incl. W03846:za60a02.r1 Homo sapiens cDNA, 5 end /clone=IMAGE-; 37986_at Cluster Incl. M60459:Human erythropoietin receptor mRNA, complete cds /; 41230_at Cluster Incl. U71087:Human MAP kinase kinase MEK5b mRNA, complete cds /; 35360_at Cluster Incl. Y17711:Homo sapiens mRNA for atopy related autoantigen CA; 32532_at Cluster Incl. L14837:Human tight junction (zonula occludens) protein ZO; 533_g_at U17418 /FEATURE= /DEFINITION=HSU17418 Human parathyroid hormone/parathy.

Metagene 401; 34438_at Cluster Incl. U71364:Human serine proteinase inhibitor (P19) mRNA, comp; 37152_at Cluster Incl. L07592:Human peroxisome proliferator activated receptor m; 38564_at Cluster Incl. U40152:Human origin recognition complex 1 (HsORC1) mRNA,; 41698_at Cluster Incl. AL031685:dJ963K23.4 (KIAA0939 (novel Sodium/hydrogen exch; 35182_f_at Cluster Incl. W25874:14e9 Homo sapiens cDNA /gb=W25874 /gi=1306015 /u; 37609_at Cluster Incl. U01833:Human nucleotide-binding protein mRNA, complete cd; 33921_at Cluster Incl. Y15409:Homo sapiens mRNA for putative glucose 6-phosphate; 1680_at D43772 /FEATURE= /DEFINITION=HUMGRB7 Human squamous cell carcinama of es; 1498_at L05148 /FEATURE= /DEFINITION=HUMTYRKIN Human protein tyrosine kinase rel; 489_at U63329 /FEATURE=cds /DEFINITION=HSU63329 Human mutY homolog (hMYH) gene,.

Metagene 402; 35094_f_at Cluster Incl. AF025527:Homo sapiens leucocyte immunoglobulin-like rec; 39221_at Cluster Incl. AF004231:Homo sapiens monocyte/macrophage Ig-related rece; 39992_at Cluster Incl. AF030302:Homo sapiens p45-BWR1A (BWR1-A) mRNA, complete c; 40439_at Cluster Incl. AF047469:Homo sapiens arsenite translocating ATPase (ASNA.

Metagene 403; 36269_at Cluster Incl. AB002364:Human mRNA for KIAA0366 gene, partial cds /cds=(; 32051_at Cluster Incl. AJ224875:Homo sapiens mRNA for putative glucosyltransfera; 33334_at Cluster Incl. X84194:H. sapiens mRNA for acylphosphatase, erythrocyte (C; 35214_at Cluster Incl. AF061016:Homo sapiens UDP-glucose dehydrogenase (UGDH) mR; 37640_at Cluster Incl. M31642:Human hypoxanthine phosphoribosyltransferase (HPRT; 38985_at Cluster Incl. AF063605:Homo sapiens brain my047 protein mRNA, complete; 40108_at Cluster Incl. D13630:Human mRNA for KIAA0005 gene, complete cds /cds=(8; 40113_at Cluster Incl. D87119:Homo sapiens mRNA for GS3955, complete cds /cds=(1; 41177_at Cluster Incl. AW024285:wt69d06.x1 Homo sapiens cDNA, 3 end /clone=IMAG; 38739_at Cluster Incl. AF017257:Homo sapiens chromosome 21 derived BAC containin; 39127_f_at Cluster Incl. X73478:H. sapiens hPTPA mRNA /cds=(189,1160) /gb=X73478; 41354_at Cluster Incl. U25997:Homo sapiens stanniocalcin precursor (STC) mRNA, c; 32607_at Cluster Incl. AF039656:Homo sapiens neuronal tissue-enriched acidic pro; 1368_at M27492 /FEATURE= /DEFINITION=HUMIL1RA Human interleukin 1 receptor mRNA,; 717_at D87119 /FEATURE= /DEFINITION=D87119 Homo sapiens mRNA for GS3955, complet.

Metagene 404; 38960_at Cluster Incl. U45975:Human phosphatidylinositol (4,5)bisphosphate 5-pho; 41103_at Cluster Incl. M91585:Human Br140 mRNA, complete cds /cds=(28,3672) /gb=; 38706_at Cluster Incl. AA552140:ng48e07.s1 Homo sapiens cDNA, 3 end /clone=IMAG; 36932_at Cluster Incl. D13636:Human mRNA for KIAA0011 gene, complete cds /cds=(3; 40602_at Cluster Incl. AF052178:Homo sapiens clone 24523 mRNA sequence /cds=UNKN; 33193_at Cluster Incl. AW052084:wy86f07.x1 Homo sapiens cDNA, 3 end /clone=IMAG.

Metagene 405; 36447_at Cluster Incl. S80990:ficolin [human, uterus, mRNA, 1736 nt]/cds=(532,1; 34010_at Cluster Incl. U90908:Human clones 23549 and 23762 mRNA, complete cds /c; 36802_at Cluster Incl. M23197:Human differentiation antigen (CD33) mRNA, complet; 37834_at Cluster Incl. Y17999:Homo sapiens mRNA for protein kinase Dyrk1B /cds=(; 38235_at Cluster Incl. AB020722:Homo sapiens mRNA for KIAA0915 protein, complete; 38516_at Cluster Incl. L10338:Human sodium channel beta-1 subunit (SCN1B) mRNA,; 40768_s_at Cluster Incl. X64228:H. sapiens can mRNA /cds=(94,6366) /gb=X64228 /gi; 33919_at Cluster Incl. AF022813:Homo sapiens tetraspan (NAG-2) mRNA, complete cd; 35350_at Cluster Incl. AB011170:Homo sapiens mRNA for KIAA0598 protein, complete; 41347_at Cluster Incl. AI814659:wj75g09.x1 Homo sapiens cDNA, 3 end /clone=IMAG; 33141_at Cluster Incl. M84472:Human 17-beta-hydroxysteroid dehydrogenase (EDH17B; 2019_s_at M68892 /FEATURE= /DEFINITION=HUMINTB7 Human integrin beta-7 subunit mR; 1673_at M14764 /FEATURE=mRNA /DEFINITION=HUMNGFR Human nerve growth factor recep.

Metagene 406; 33252_at Cluster Incl. D38073:Human mRNA for hRlf beta subunit (p102 protein), c; 37927_at Cluster Incl. X12654:Human mRNA for cell cycle gene RCC1 /cds=(182,1447; 32180_s_at Cluster Incl. AB000461:Homo sapiens mRNA, complete cds, clone-RES4-22; 646_s_at L29218 /FEATURE=mRNA /DEFINITION=HUMCLK2B Homo sapiens clk2 mRNA, compl.

Metagene 407; 35858_at Cluster Incl. AA996066:os33d01.s1 Homo sapiens cDNA, 3 end /clone=IMAG; 39668_at Cluster Incl. X95694:H. sapiens mRNA for AP-2 beta transcription factor; 40645_at Cluster Incl. L33801:Human protein kinase mRNA, complete cds /cds=(39,1; 36524_at Cluster Incl. AB029035:Homo sapiens mRNA for KIAA1112 protein, partial; 36538_at Cluster Incl. AB018314:Homo sapiens mRNA for KIAA0771 protein, partial; 32192_g_at Cluster Incl. D13969:Human mRNA for Mel-18 protein, complete cds /cds.

Metagene 408; 34907_at Cluster Incl. AB014541:Homo sapiens mRNA for KIAA0641 protein, complete; 35008_at Cluster Incl. AB002345:Human mRNA for KIAA0347 gene, complete cds /cds=; 35379_at Cluster Incl. X54412:Human mRNA for alpha1(IX) collagen (long form) /cd; 38209_at Cluster Incl. L22647:Human prostaglandin receptor ep1 subtype mRNA, com; 38944_at Cluster Incl. U68019:Homo sapiens mad protein homolog (hMAD-3) mRNA, co; 33293_at Cluster Incl. AB023167:Homo sapiens mRNA for KIAA0950 protein, partial; 34682_at Cluster Incl. AL050271:Homo sapiens mRNA; cDNA DKFZp566H0824 (from clon; 37639_at Cluster Incl. X07732:Human hepatoma mRNA for serine protease hepsin /cd; 36581_at Cluster Incl. U09510:Human glycyl-tRNA synthetase mRNA, complete cds /c; 38093_at Cluster Incl. U90909:Human clone 23722 mRNA sequence /cds=UNKNOWN /gb=U; 39547_at Cluster Incl. AB008515:Homo sapiens mRNA for RanBPM, complete cds /cds=; 713_at Helix-Loop-Helix Protein Delta Max, Alt. Splice 1; 416_s_at X61755 /FEATURE=mRNA /DEFINITION=HSHOX3D Human HOX3D gene for homeoprot

Metagene 409; 106_at Z35278 /FEATURE=mRNA /DEFINITION=HSAML1RN H. sapiens PEBP2aC1 acute myeloi.

Metagene 410; 31952_at Cluster Incl. X69391:H. sapiens mRNA for ribosomal protein L6 /cds=(26,8; 32433_at Cluster Incl. Z97353:Human DNA sequence from clone 90L6 on chromosome 2; 32436_at Cluster Incl. U14968:Human ribosomal protein L27a mRNA, complete cds /c; 34570_at Cluster Incl. S79522:ubiquitin carboxyl extension protein [human, mRNA,; 34646_at Cluster Incl. Z25749:H. sapiens gene for ribosomal protein S7 /cds=(81,6; 35508_at Cluster Incl. X79781:H. sapiens ray mRNA /cds=(81,686) /gb=X79781 /gi=76; 32883_at Cluster Incl. X77744:H. sapiens F11 mRNA /cds=UNKNOWN /gb=X77744 /gi=456; 39298_at Cluster Incl. AB022918:Homo sapiens mRNA for alpha2,3-sialyltransferase; 31899_at Cluster Incl. D14659:Human mRNA for KIAA0103 gene, complete cds /cds=(6; 34773_at Cluster Incl. AF038952:Homo sapiens cofactor A protein mRNA, complete c; 35642_at Cluster Incl. AF053551:Homo sapiens metaxin 2 (MTX2) mRNA, nuclear gene; 36517_at Cluster Incl. M96982:Homo sapiens U2 snRNP auxiliary factor small subun; 37242_at Cluster Incl. U79260:Human clone 23745 mRNA, complete cds /cds=(609,102; 38318_at Cluster Incl. AL050128:Homo sapiens mRNA; cDNA DKFZp586G051 (from clone; 39757_at Cluster Incl. J04621:Human heparan sulfate proteoglycan (HSPG) core pro; 40411_at Cluster Incl. D80003:Human mRNA for KIAA0181 gene, partial cds /cds=(0,; 40467_at Cluster Incl. AB006202:Homo sapiens mRNA for cytochrome b small subunit; 40811_at Cluster Incl. AB011148:Homo sapiens mRNA for KIAA0576 protein, partial; 41156_g_at Cluster Incl. U03100:Human alpha2(E)-catenin mRNA, complete cds /cds=; 41746_at Cluster Incl. Z83840:Human DNA sequence from clone 216E10 on chromosome; 41768_at Cluster Incl. M33336:Human cAMP-dependent protein kinase type I-alpha s; 41785_at Cluster Incl. U73824:Human p97 mRNA, complete cds /cds=(306,3029) /gb=U; 32789_at Cluster Incl. AA149428:zl26a05.s1 Homo sapiens cDNA, 3 end /clone=IMAG; 32833_at Cluster Incl. M59287:Human protein kinase mRNA /cds=UNKNOWN /gb=M59287; 33351_at Cluster Incl. AF064607:Homo sapiens GC20 protein mRNA, complete cds /cd; 33817_at Cluster Incl. S63912:D10S102=FBRNP [human, fetal brain, mRNA, 3043 nt]; 33891_at Cluster Incl. AL080061:Homo sapiens mRNA; cDNA DKFZp564H182 (from clone; 34307_at Cluster Incl. U81006:Human p76 mRNA, complete cds /cds=(133,2124) /gb=U; 35738_at Cluster Incl. AI347088:qp60d09.x1 Homo sapiens cDNA, 3 end /clone=IMAG; 35821_at Cluster Incl. U75697:Human transcription regulator RPD3-2B mRNA, comple; 35842_at Cluster Incl. AL049265:Homo sapiens mRNA; cDNA DKFZp564F053 (from clone; 36098_at Cluster Incl. M72709:Human alternative splicing factor mRNA, complete c; 36601_at Cluster Incl. M33308:Human vinculin mRNA, complete cds /cds=(50,3250) /; 37308_at Cluster Incl. AI888084:wm29g09.x1 Homo sapiens cDNA, 3 end /clone=IMAG; 37373_at Cluster Incl. U27460:Human uridine diphosphoglucose pyrophosphorylase m; 37677_at Cluster Incl. V00572:Human mRNA encoding phosphoglycerate kinase /cds=(; 37707_i_at Cluster Incl. M81118:Human alcohol dehydrogenase chi polypeptide (ADH; 37717_at Cluster Incl. L03532:Human M4 protein mRNA, complete cds /cds=(11,2200); 38837_at Cluster Incl. W26226:22e3 Homo sapiens cDNA /gb=W26226 /gi=1306637 /ug=; 39839_at Cluster Incl. M24069:Human DNA-binding protein A (dbpA) gene, 3 end /c; 40916_at Cluster Incl. AL035494:Human DNA sequence from clone 635G19 on chromoso; 40961_at Cluster Incl. X72889:H. sapiens hbrm mRNA /cds=(222,4982) /gb=X72889 /gi; 41292_at Cluster Incl. L22009:Human hnRNP H mRNA, complete cds /cds=(72,1421) /g; 1450_g_at D00763 /FEATURE= /DEFINITION=HUMPSC9 Human mRNA for proteasome subunit; 1295_at L19067 /FEATURE= /DEFINITION=HUMNFKB65A Human NF-kappa-B transcription f; 1250_at U47077 /FEATURE= /DEFINITION=HSU47077 Homo sapiens DNA-dependent protein; 950_at D87127 /FEATURE= /DEFINITION=D87127 Homo sapiens mRNA for translocation p; 853_at S74017 /FEATURE= /DEFINITION=S74017 Nrf2=NF-E2-like basic leucine zipper; 292_s_at Protein Kinase; 140_s_at U68063 /FEATURE= /DEFINITION=HSU68063 Human transformer-2 beta (htra-2.

Metagene 411; 32402_s_at Cluster Incl. Y10931:H. sapiens mRNA for symplekin /cds=(459,3887) /gb; 35190_at Cluster Incl. AL031447:dJ126A5.2.1 (novel protein) (isoform 1) /cds=(0,; 36828_at Cluster Incl. AB002324:Human mRNA for KIAA0326 gene, partial cds /cds=(; 37903_at Cluster Incl. L25665:Human GTP-binding protein (HSR1) mRNA, complete cd; 39004_at Cluster Incl. AI432190:tg77f11.x1 Homo sapiens cDNA, 3 end /clone=IMAG; 39372_at Cluster Incl. W26480:30b8 Homo sapiens cDNA /gb=W26480 /gi=1307179 /ug=; 34310_at Cluster Incl. Y00486:Human APRT gene for adenine phosphoribosyltransfer; 34845_at Cluster Incl. AL035398:Human DNA sequence from clone 796117 on chromoso; 36658_at Cluster Incl. D13643:Human mRNA for KIAA0018 gene, complete cds /cds=(3; 36984_f_at Cluster Incl. X89214:H. sapiens mRNA for haptoglobin related protein /; 37739_at Cluster Incl. M86737:Human high mobility group box (SSRP1) mRNA, comple.

Metagene 412; 40367_at Cluster Incl. M22489:Human bone morphogenetic protein 2A (BMP-2A) mRNA; 41051_at Cluster Incl. X95073:H. sapiens mRNA for translin associated protein X /; 32099_at Cluster Incl. D50928:Human mRNA for KIAA0138 gene, complete cds /cds=(3; 32659_at Cluster Incl. AL050109:Homo sapiens mRNA; cDNA DKFZp586J0119 (from clon; 32695_at Cluster Incl. Z97632:dJ196E23.2 (HIV-1 transcriptional elongation facto; 34685_at Cluster Incl. AI685944:tu38g02.x1 Homo sapiens cDNA, 3 end /clone=IMAG; 36083_at Cluster Incl. U01160:Human transmembrane 4 superfamily protein (SAS) mR; 37199_at Cluster Incl. AI760932:wi70d01.x1 Homo sapiens cDNA, 3 end /clone=IMAG; 38669_at Cluster Incl. D86959:Human mRNA for KIAA0204 gene, complete cds /cds=(5; 38685_at Cluster Incl. AL035306:H. sapiens gene from PAC 42616, similar to syntax; 38983_at Cluster Incl. AI223047:qg70a09.x1 Homo sapiens cDNA, 3 end /clone=IMAG; 39784_at Cluster Incl. U26032:Human translation initiation factor eIF-2alpha mRN; 40418_at Cluster Incl. X74262:H. sapiens RbAp48 mRNA encoding retinoblastoma bind; 40437_at Cluster Incl. AL049944:Homo sapiens mRNA; cDNA DKFZp564G2022 (from clon; 41737_at Cluster Incl. AF048977:Homo sapiens Ser/Arg-related nuclear matrix prot; 41754_at Cluster Incl. M92439:Human leucine-rich protein mRNA, complete cds /cds; 32205_at Cluster Incl. AF072860:Homo sapiens protein activator of the interferon; 32234_at Cluster Incl. AF007871:Homo sapiens torsinA (DYT1) mRNA, complete cds /; 33420_g_at Cluster Incl. U83857:Human Aac11 (aac11) mRNA, complete cds /cds=(77,; 36111_s_at Cluster Incl. X75755:H. sapiens PR264 gene /cds=(98,763) /gb=X75755 /g; 36968_s_at Cluster Incl. AL050353:Homo sapiens mRNA; cDNA DKFZp547C0410 (from cl; 38380_at Cluster Incl. Y18863:Homo sapiens mRNA for ribonuclease P protein subun; 38410_at Cluster Incl. X72964:H. sapiens mRNA for caltractin /cds=(47,565) /gb=X7; 38817_at Cluster Incl. AF047437:Homo sapiens sperm acrosomal protein mRNA, compl; 41830_at Cluster Incl. AB007963:Homo sapiens mRNA for KIAA0494 protein, complete; 33175_at Cluster Incl. AA156237:zl50c09.s1 Homo sapiens cDNA, 3 end /clone=IMAG; 2060_at M13995 /FEATURE=mRNA /DEFINITION=HUMBCL2B Human B-cell leukemia/lymphoma; 1211_s_at U84388 /FEATURE= /DEFINITION=HSU84388 Human death domain containing pr; 1017_at U73737 /FEATURE=mRNA /DEFINITION=HUMMSH06 Human hMSH6 gene, exons 6-10 a; 585_at M30938 /FEATURE=mRNA#2 /DEFINITION=HUMKUP Human Ku (p70 /p80) subunit mRNA; 507_s_at U43189 /FEATURE= /DEFINITION=HSU43189 Human Ets transcription factors N; 174_s_at U61167 /FEATURE= /DEFINITION=HSU61167 Human SH3 domain-containing prote.

Metagene 413; 35524_at Cluster Incl. U08198:Human complement C8 gamma subunit precursor (C8G) 32975_g_at Cluster Incl. U07563:Human ABL gene, exon 1b and intron 1b, and putat; 37451_at Cluster Incl. AL109695:Homo sapiens mRNA full length insert cDNA clone; 38171_at Cluster Incl. U94747:Human WD repeat protein HAN11 mRNA, complete cds /; 39223_at Cluster Incl. AL096749:Homo sapiens mRNA; cDNA DKFZp434G153 (from clone; 39934_at Cluster Incl. AB023061:Homo sapiens mRNA for small GTP-binding protein; 36121_at Cluster Incl. AB028988:Homo sapiens mRNA for KIAA1065 protein, complete; 40207_g_at Cluster Incl. AI767675:wh38d11.x1 Homo sapiens cDNA, 3 end /clone=IM; 40641_at Cluster Incl. AF038362:Homo sapiens TBP-associated factor 172 (TAF-172).

Metagene 414; 34651_at Cluster Incl. M58525:Homo sapiens catechol-O-methyltransferase (COMT) m; 35176_at Cluster Incl. Z82022:H. sapiens mRNA for GlcNac-1-P transferase /cds=(10; 33863_at Cluster Incl. U65785:Human 150 kDa oxygen-regulated protein ORP150 mRNA; 39113_at Cluster Incl. AI262789:qk35e02.x1 Homo sapiens cDNA, 3 end /clone=IMAG; 40932_at Cluster Incl. H18080:ym38h10.s1 Homo sapiens cDNA, 3 end /clone=IMAGE-; 32543_at Cluster Incl. M84739:Human autoantigen calreticulin mRNA, complete cds; 2075_s_at L36719 /FEATURE=mRNA /DEFINITION=HUMMKK3A Homo sapiens MAP kinase kina; 1752_at AD000092 /FEATURE=cds#6 /DEFINITION=CH19HHR23 Homo sapiens DNA from chro; 1438_at X75208 /FEATURE=cds /DEFINITION=HSPTKR H. sapiens HEK2 mRNA for protein t; 442_at X15187 /FEATURE=cds /DEFINITION=HSTRA1 Human tra1 mRNA for human homologu.

Metagene 415; 37978_at Cluster Incl. D78177:Homo sapiens mRNA for quinolinate phosphoribosyl t; 32249_at Cluster Incl. M65292:Human factor H homologue mRNA, complete cds /cds=(; 33867_s_at Cluster Incl. X77494:H. sapiens MSSP-2 mRNA /cds=(231,1400) /gb=X77494; 35848_at Cluster Incl. AL049432:Homo sapiens mRNA; cDNA DKFZp586J231 (from clone; 40626_at Cluster Incl. AI693193:wd68f02.x1 Homo sapiens cDNA, 3 end /clone=IMAG; 1846_at L78132 /FEATURE= /DEFINITION=HUMPCTA1A Human prostate carcinoma tumor an; 332_at Zinc Finger Protein Kup; 166_at U48405 /FEATURE=cds /DEFINITION=HSU48405 Human G protein coupled receptor.

Metagene 416; 32487_s_at Cluster Incl. AB002533:Homo sapiens mRNA for Qip1, complete cds /cds=; 34445_at Cluster Incl. AB007940:Homo sapiens mRNA for KIAA0471 protein, complete; 36695_at Cluster Incl. AI290480:ql98b12.x1 Homo sapiens cDNA, 3 end /clone=IMAG; 36730_at Cluster Incl. U66684:HSU66684 Homo sapiens cDNA /gb=U66684 /gi=1906569; 37422_at Cluster Incl. S71018:cyclophilin C [human, kidney, mRNA, 883 nt]/cds=(; 38220_at Cluster Incl. U20938:Human lymphocyte dihydropyrimidine dehydrogenase m; 39211_at Cluster Incl. AF091080:Homo sapiens clone 614 unknown mRNA, complete se; 40349_at Cluster Incl. AL049442:Homo sapiens mRNA; cDNA DKFZp586N1720 (from clon; 40399_r_at Cluster Incl. AI743406:wg92g12.x1 Homo sapiens cDNA, 3 end /clone=IM; 31892_at Cluster Incl. X58288:H. sapiens hR-PTPu gene for protein tyrosine phosph; 32696_at Cluster Incl. X59841:Human PBX3 mRNA /cds=UNKNOWN /gb=X59841 /gi=35314; 33267_at Cluster Incl. AF035315:Homo sapiens clone 23664 and 23905 mRNA sequence; 34759_at Cluster Incl. U68494:Human hbc647 mRNA sequence /cds=UNKNOWN /gb=U68494; 35193_at Cluster Incl. AF060219:Homo sapiens RCC1-like G exchanging factor RLG m; 35677_at Cluster Incl. AL035369:H. sapiens novel gene from PAC 117P20, chromosome; 35682_at Cluster Incl. AI133727:Habcs0217 Homo sapiens cDNA /gb=AI133727 /gi=360; 36061_at Cluster Incl. AF009314:Homo sapiens clone TUA8 Cri-du-chat region mRNA; 36069_at Cluster Incl. AB007925:Homo sapiens mRNA for KIAA0456 protein, partial; 36821_at Cluster Incl. AL050367:Homo sapiens mRNA; cDNA DKFZp564A026 (from clone; 37229_at Cluster Incl. U49844:Human FRAP-related protein (FRP1) mRNA, complete c; 37280_at Cluster Incl. U59912:Human chromosome 4 Mad homolog Smad1 mRNA, complet; 37571_at Cluster Incl. AB028981:Homo sapiens mRNA for KIAA1058 protein, partial; 38306_at Cluster Incl. AA477576:zu44b03.r1 Homo sapiens cDNA, 5 end /clone=IMAG; 38364_at Cluster Incl. AF068197:Homo sapiens BCE-1 mRNA, complete cds /cds=(516,; 39741_at Cluster Incl. D16481:Homo sapiens mRNA for mitochondrial 3-ketoacyl-CoA; 39771_at Cluster Incl. AB018283:Homo sapiens mRNA for KIAA0740 protein, complete; 40516_at Cluster Incl. L19872:Human AH-receptor mRNA, complete cds /cds=(375,292; 40805_at Cluster Incl. AB007900:Homo sapiens KIAA0440 mRNA, partial cds /cds=(0,; 40818_at Cluster Incl. D14041:Homo sapiens mRNA for H-2K binding factor-2, compl; 41763_g_at Cluster Incl. D64015:Homo sapiens mRNA for T-cluster binding protein,; 32218_at Cluster Incl. AF034176:AF034176 Homo sapiens cDNA /clone=ntcon5-contig; 36689_at Cluster Incl. AL040446:DKFZp434D1414_r1 Homo sapiens cDNA, 5 end /clon; 37733_at Cluster Incl. L35263:Human CSaids binding protein (CSBP1) mRNA, complet; 38062_at Cluster Incl. D87467:Human mRNA for KIAA0277 gene, complete cds /cds=(5; 38411_at Cluster Incl. U90916:Human clone 23815 mRNA sequence /cds=UNKNOWN /gb=U; 41335_at Cluster Incl. AL050084:Homo sapiens mRNA; cDNA DKFZp566O1646 (from clon; 32510_at Cluster Incl. AF026947:Homo sapiens aflatoxin aldehyde reductase AFAR m; 32576_at Cluster Incl. U94855:Homo sapiens translation initiation factor 3 47 kD; 33102_at Cluster Incl. D67031:Homo sapiens ADDL mRNA for adducin-like protein, c; 1596_g_at L06139 /FEATURE= /DEFINITION=HUMTEKRPTK Homo sapiens receptor protein-; 995_g_at X58288 /FEATURE=mRNA /DEFINITION=HSHRPTPU H. sapiens hR-PTPu gene for pr.

Metagene 417; 31463_s_at Cluster Incl. AL022097:Homo sapiens DNA sequence from PAC 256G22 on c; 36443_at Cluster Incl. X99947:Homo sapiens mRNA dynein-related protein /cds=(529; 33485_at Cluster Incl. D23660:Human mRNA for ribosomal protein, complete cds /cd; 39995_s_at Cluster Incl. U13395:Human oxidoreductase (HHCMA56) mRNA, complete cd; 40395_at Cluster Incl. AB007932:Homo sapiens mRNA for KIAA0463 protein, partial; 41408_at Cluster Incl. AF042169:Homo sapiens putative ATP-dependent mitochondria; 41719_i_at Cluster Incl. AF009767:AF009767 Homo sapiens cDNA /clone=C97A-12 /gb=; 32724_at Cluster Incl. AF023462:Homo sapiens peroxisomal phytanoyl-CoA alpha-hyd; 33223_at Cluster Incl. AB011133:Homo sapiens mRNA for KIAA0561 protein, partial; 36073_at Cluster Incl. U35139:Human NECDIN related protein mRNA, complete cds /c; 37980_at Cluster Incl. U03644:Human recepin mRNA, complete cds /cds=(32,1387) /g; 38689_at Cluster Incl. AL021937:dJ149A16.6 (novel protein, human ortholog of wor; 39367_at Cluster Incl. AA522537:ni38e08.s1 Homo sapiens cDNA, 3 end /clone=IMAG; 40139_at Cluster Incl. U88966:Human protein rapamycin associated protein (FRAP2); 40486_g_at Cluster Incl. AA176780:zp32a10.s1 Homo sapiens cDNA, 3 end /clone=IM; 41765_at Cluster Incl. AI541285:pec1.2-4.D10.r Homo sapiens cDNA, 5 end /clone_{—}; 32173_at Cluster Incl. X95384:Homo sapiens mRNA for translational inhibitor prot; 32745_at Cluster Incl. AF034091:Homo sapiens nuclear localization signal contain; 33359_at Cluster Incl. AB018311:Homo sapiens mRNA for KIAA0768 protein, partial; 34299_at Cluster Incl. AL096880:Novel human mRNA containing Zinc finger C2H2 typ; 34391_at Cluster Incl. Y08915:H. sapiens mRNA for alpha 4 protein /cds=(8,1027) /; 34826_at Cluster Incl. L21936:Human succinate dehydrogenase flavoprotein subunit; 34828_at Cluster Incl. AL037557:DKFZp564H2472_r1 Homo sapiens cDNA, 5 end /clon; 35358_at Cluster Incl. AB028998:Homo sapiens mRNA for KIAA1075 protein, partial; 35760_at Cluster Incl. AF087135:Homo sapiens FIFO-type ATPase subunit d mRNA, nu; 35836_at Cluster Incl. AB019408:Homo sapiens mRNA, expressed in fibroblasts of p; 36137_at Cluster Incl. X86691:H. sapiens mRNA for 218 kD Mi-2 protein /cds=(89,582; 37002_at Cluster Incl. D32143:Human mRNA for biliverdin-IXbeta reductase I /cds=; 37704_at Cluster Incl. Z14093:H. sapiens mRNA for branched chain decarboxylase a1; 37766_s_at Cluster Incl. AF035309:Homo sapiens clone 23598 mRNA, complete cds /c; 38762_at Cluster Incl. AF083255:Homo sapiens RNA helicase-related protein mRNA,; 39184_at Cluster Incl. AI857469:wl57f04.x1 Homo sapiens cDNA, 3 end /clone=IMAG; 41302_at Cluster Incl. R59606:yh02e06.s1 Homo sapiens cDNA, 3 end /clone=IMAGE-; 326_i_at Ribosomal Protein S20.

Metagene 418; 34969_s_at Cluster Incl. AL096750:Homo sapiens mRNA; cDNA DKFZp434H244 (from clo; 38219_at Cluster Incl. D10656:Human mRNA for CRK-II, complete cds /cds=(105,1019; 34656_at Cluster Incl. X82895:H. sapiens mRNA for DLG2 /cds=(87,1817) /gb=X82895; 35198_at Cluster Incl. AF070596:Homo sapiens clone 24796 mRNA sequence /cds=UNKN; 40485_at Cluster Incl. AA176780:zp32a10.s1 Homo sapiens cDNA, 3 end /clone=IMAG; 32611_at Cluster Incl. X75252:H. sapiens phosphatidylethanolamine binding protein; 160033_s_at NM_{—}006297 /FEATURE=mRNA /DEFINITION=Homo sapiens X-ray repair comple.

Metagene 419; 40425_at Cluster Incl. M57730:Human B61 mRNA, complete cds /cds=(73,690) /gb=M57.

Metagene 420; 33651_at Cluster Incl. AB013456:Homo sapiens hAQP8 mRNA for aquaporin 8, complet; 39567_at Cluster Incl. AB006190:Homo sapiens mRNA for aquaporin adipose, complet; 39051_at Cluster Incl. U31767:Human neuronatin alpha and neuronatin beta genes,; 32507_at Cluster Incl. AW043925:wy82b07.x1 Homo sapiens cDNA, 3 end /clone=IMAG; 1754_at AF006041 /FEATURE= /DEFINITION=AF006041 Homo sapiens Fas-binding protein; 1641_s_at U32986 /FEATURE= /DEFINITION=HSU32986 Human xeroderma pigmentosum grou; 1001_at X60957 /FEATURE=cds /DEFINITION=HSTIEMR Human tie mRNA for putative rece; 485_at U59748 /FEATURE= /DEFINITION=HSU59748 Human desert hedgehog (hDHH) mRNA,; 145_s_at U80987 /FEATURE= /DEFINITION=HSU80987 Human transcription factor TBX5 m; 107_at Z95624 /FEATURE=cds /DEFINITION=HSU237H1 Human DNA sequence from cosmid U.

Metagene 421; 35106_at Cluster Incl. Z46967:H. sapiens mRNA for calicin (partial) /cds=(0,1757); 35390_at Cluster Incl. AI651024:wa96h06.x1 Homo sapiens cDNA, 3 end /clone=IMAG; 35419_g_at Cluster Incl. J04178:Human abnormal beta-hexosaminidase alpha chain (; 38551_at Cluster Incl. U52112:neural cell adhesion molecule L1 /cds=(19,3792) /g; 40762_g_at Cluster Incl. AA705628:zf40a01.s1 Homo sapiens cDNA, 3 end /clone=37; 38005_at Cluster Incl. AJ005866:Homo sapiens mRNA for putative Sqv-7-like protei; 38691_s_at Cluster Incl. J03553:Human pulmonary surfactant protein (SP5) mRNA, c; 32768_at Cluster Incl. AL048308:DKFZp586A2224_s1 Homo sapiens cDNA /clone=DKFZp5; 35266_at Cluster Incl. AL049288:Homo sapiens mRNA; cDNA DKFZp564M053 (from clone; 35809_g_at Cluster Incl. AL031681:dJ862K6.2.2 (splicing factor, arginine/serine-; 38454_g_at Cluster Incl. X15606:Human mRNA for ICAM-2, cell adhesion ligand for; 1461_at M69043 /FEATURE= /DEFINITION=HUMMAD3A Homo sapiens MAD-3 mRNA encoding I; 1347_at S78187 /FEATURE= /DEFINITION=S78187 CDC25Hu2=cdc25+homolog [human, mRNA.

Metagene 422; 32319_at Cluster Incl. AL022310:dJ395P12.2 (tax-transcriptionally activated glyc; 34454_r_at Cluster Incl. U32576:Human apolipoprotein apoC-IV (APOC4) gene, compl; 37417_at Cluster Incl. M36542:Human lymphoid-specific transcription factor mRNA,; 39645_r_at Cluster Incl. AF033105:Homo sapiens X-arrestin mRNA, complete cds /cd; 41442_at Cluster Incl. AB010419:Homo sapiens mRNA for MTG8-related protein MTG16; 33737_f_at Cluster Incl. AI871359:wl81b11.x1 Homo sapiens cDNA, 3 end /clone=IM; 40233_at Cluster Incl. AI198214:qi55c03.x1 Homo sapiens cDNA, 3 end /clone=IMAG.

Metagene 423; 31906_at Cluster Incl. AF068754:Homo sapiens heat shock factor binding protein 1; 32316_s_at Cluster Incl. X15183:Human mRNA for 90-kDa heat-shock protein /cds=(6; 41450_at Cluster Incl. AI553878:tn30a05.x1 Homo sapiens cDNA, 3 end /clone=IMAG; 34678_at Cluster Incl. AL096713:Homo sapiens mRNA; cDNA DKFZp564E1616 (from clon; 35236_g_at Cluster Incl. AA099265:zk84f07.r1 Homo sapiens cDNA, 5 end /clone=IM; 38331_at Cluster Incl. Y07566:H. sapiens mRNA for RIT protein /cds=(145,804) /gb=; 38684_at Cluster Incl. AJ010953:Homo sapiens mRNA for putative Ca2+-transporting; 39756_g_at Cluster Incl. Z93930:Human DNA sequence from clone 292E10 on chromoso; 36614_at Cluster Incl. X87949:H. sapiens mRNA for BiP protein /cds=(222,2183) /gb; 37720_at Cluster Incl. M22382:Human mitochondrial matrix protein P1 (nuclear enc; 39118_at Cluster Incl. L08069:Human heat shock protein, E. coli DnaJ homologue m; 1957_s_at AF054598 /FEATURE=mRNA /DEFINITION=HSTGFBR1G9 Homo sapiens TGF-beta ty; 1262_s_at M19154 /FEATURE=mRNA /DEFINITION=HUMTGFB2A Human transforming growth f; 1161_at J04988 /FEATURE=cds /DEFINITION=HUMHSP90B Human 90 kD heat shock protein; 1102_s_at M10901 /FEATURE=mRNA /DEFINITION=HHUMGCRA Human glucocorticoid receptor; 809_at U57094 /FEATURE= /DEFINITION=HSU57094 Human small GTP-binding protein mRN; 714_at Adenylyl Cyclase-Associated Protein 2; 276_at L08069 /FEATURE= /DEFINITION=HUMDNAJHOM Human heat shock protein, E. coli.

Metagene 424; 31736_at Cluster Incl. AA975427:oq28g02.s1 Homo sapiens cDNA, 3 end /clone=IMAG; 31944_at Cluster Incl. AI028290:ov84f11.x1 Homo sapiens cDNA, 3 end /clone=IMAG; 33690_at Cluster Incl. AL080190:Homo sapiens mRNA; cDNA DKFZp434A202 (from clone; 32872_at Cluster Incl. AL049279:Homo sapiens mRNA; cDNA DKFZp564I083 (from clone; 36707_s_at Cluster Incl. X89059:H. sapiens mRNA for unknown protein expressed in; 40725_at Cluster Incl. AF047438:Homo sapiens GOS28 /P28 protein mRNA, complete cd; 41388_at Cluster Incl. AF017418:Homo sapiens homeobox protein MEIS2 (MEIS2) mRNA; 41438_at Cluster Incl. AL049923:Homo sapiens mRNA; cDNA DKFZp547E2210 (from clon; 32064_at Cluster Incl. Y13467:Homo sapiens mRNA for RB18A protein /cds=(235,4935; 32127_at Cluster Incl. U90030:Homo sapiens bicaudal-D (BICD) mRNA, alternatively; 32643_at Cluster Incl. L07956:Homo sapiens 1,4-alpha-glucan branching enzyme (HG; 32662_at Cluster Incl. AL041663:DKFZp434M0217_s1 Homo sapiens cDNA, 3 end /clon; 34697_at Cluster Incl. AF074264:Homo sapiens LDL receptor-related protein 6 (LRP; 37984_s_at Cluster Incl. M57763:Human ADP-ribosylation factor (hARF6) mRNA, comp; 32802_at Cluster Incl. AB011169:Homo sapiens mRNA for KLAA0597 protein, partial; 33418_at Cluster Incl. AL096752:Homo sapiens mRNA; cDNA DKFZp434A012 (from clone; 33845_at Cluster Incl. W28483:47e11 Homo sapiens cDNA /gb=W28483 /gi=1308431 /ug; 36991_at Cluster Incl. L14076:Human pre-mRNA splicing factor SRp75 mRNA, complet; 37710_at Cluster Incl. L08895:Homo sapiens MADS/MEF2-family transcription factor; 40191_s_at Cluster Incl. AI761647:wg66h09.x1 Homo sapiens cDNA, 3 end /clone=IM; 33188_at Cluster Incl. U37221:Human cyclophilin-like protein mRNA, partial cds /; 1383_at M64929 /FEATURE= /DEFINITION=HUMPROP2AA Human protein phosphatase 2A alp.

Metagene 425; 31745_at Cluster Incl. AF007194:Homo sapiens mucin (MUC3) mRNA, partial cds /cds; 32007_at Cluster Incl. W29045:55e12 Homo sapiens cDNA /gb=W29045 /gi=1309002 /ug; 36355_at Cluster Incl. M13903:Human involucrin mRNA /cds=(0,1757) /gb=M13903 /gi; 35913_at Cluster Incl. U88047:Homo sapiens DNA binding protein homolog (DRIL1) m; 38205_at Cluster Incl. AB021742:Homo sapiens NDRF gene for neuroD-related factor; 40700_at Cluster Incl. U36500:Human lymphoid-specific SP100 homolog (LYSP100-B); 41050_at Cluster Incl. X95525:H. sapiens mRNA for TAFII100 protein /cds=(23,2422); 41647_at Cluster Incl. W28742:51a7 Homo sapiens cDNA /gb=W28742 /gi=1308690 /ug=; 32710_at Cluster Incl. X83127:H. sapiens mRNA for voltage gated potassium channel; 33744_at Cluster Incl. AL080150:Homo sapiens mRNA; cDNA DKFZp434D174 (from clone; 35687_at Cluster Incl. Z24459:H. sapiens MTCP1 gene, exons 2A to 7 (and joined mR; 35990_at Cluster Incl. AB007947:Homo sapiens mRNA for KIAA0478 protein, complete; 38629_at Cluster Incl. AF047863:untitled /cds=(17,1075) /gb=AF047863 /gi=2909668; 32810_at Cluster Incl. AF019369:untitled /cds=(89,826) /gb=AF019369 /gi=2623563; 39834_at Cluster Incl. X66403:H. sapiens mRNA for acetylcholine receptor (epsilon; 1684_s_at X69950 /FEATURE=exon#3 /DEFINITION=HSWT1WIT H. sapiens DNA sequence for; 1689_at AB000895 /FEATURE= /DEFINITION=AB000895 Homo sapiens mRNA for cadherin F; 1339_s_at X14675 /FEATURE=cds /DEFINITION=HSBCR3C Human bcr-abl mRNA 5 fragment; 1116_at M28170 /FEATURE= /DEFINITION=HUMCSPC Human cell surface protein CD19 (CD.

Metagene 426; 33467_at Cluster Incl. X66171:H. sapiens CMRF35 mRNA, complete CDS /cds=(239,913); 33503_at Cluster Incl. W29105:56d8 Homo sapiens cDNA /gb=W29105 /gi=1309071 /ug=; 35402_at Cluster Incl. AF068868:Homo sapiens TNFR-related death receptor-6 (DR6); 35869_at Cluster Incl. AB020499:Homo sapiens BCG-regulated mRNA for MD-1 homolog; 40331_at Cluster Incl. AF035819:Homo sapiens macrophage receptor MARCO mRNA, com; 41677_at Cluster Incl. AF035279:Homo sapiens clone 23888 mRNA sequence /cds=(162; 36472_at Cluster Incl. U32849:Homo sapiens Nmi mRNA, complete cds /cds=(280,1203; 39061_at Cluster Incl. D28137:Human mRNA for BST-2, complete cds /cds=(9,551) /g; 39721_at Cluster Incl. U09303:Human T cell leukemia LERK-2 (EPLG2) mRNA, complet; 37749_at Cluster Incl. D78611:Human MEST mRNA, complete cds /cds=(223,1230) /gb=; 1939_at M22898 /FEATURE=mRNA /DEFINITION=HUMP53A11 Human phosphoprotein p53 gene; 1890_at AB000584 /FEATURE= /DEFINITION=AB000584 Homo sapiens mRNA for TGF-beta s; 626_s_at L78833 /FEATURE=cds#5 /DEFINITION=HUMBRCA1 Human BRCA1, Rho7 and vatI g.

Metagene 427; 35446_at Cluster Incl. AA767013:oa42a08.s1 Homo sapiens cDNA /clone=IMAGE-130760; 41222_at Cluster Incl. AF067575:untitled /cds=(21,2564) /gb=AF067575 /gi=3789867; 1326_at U60519 /FEATURE= /DEFINITION=HSU60519 Human apoptotic cysteine protease; 574_s_at M87507 /FEATURE= /DEFINITION=HUMIL1BCE Homo sapien interleukin-1 beta c.

Metagene 428; 38278_at Cluster Incl. M62324:Human modulator recognition factor I (MRF-1) mRNA,; 39052_at Cluster Incl. J00124:Homo sapiens 50 kDa type I epidermal keratin gene,; 32855_at Cluster Incl. L00352:Human low density lipoprotein receptor gene /cds=(; 35850_at Cluster Incl. AI950382:wp10g06.x1 Homo sapiens cDNA, 3 end /clone=IMAG; 41828_at Cluster Incl. Y10746:H. sapiens mRNA for protein containing MBD 1 /cds=(; 1842_at Oncogene Tls/Chop, Fusion Activated; 287_at L19871 /FEATURE= /DEFINITION=HUMATF3X Human activating transcription fact.

Metagene 429; 33045_r_at Cluster Incl. AI830463:wh51e03.x1 Homo sapiens cDNA, 3 end /clone=IM; 32928_at Cluster Incl. AJ012214:Homo sapiens mRNA for PLA-1 protein /cds=(0,1310; 39611_at Cluster Incl. AI557322:PT2.1_{—}16_F11.r Homo sapiens cDNA, 3 end /clone_{—}; 37201_at Cluster Incl. D38535:Human mRNA for PK-120 /cds=(129,2921) /gb=D38535 /; 37653_at Cluster Incl. AF018081:Homo sapiens type XVIII collagen (COL18A1) mRNA,; 1400_at M13207 /FEATURE=expanded-cds /DEFINITION=HUMCSFGMA Human granulocyte-mac.

Metagene 430; 37432_g_at Cluster Incl. AF077953:Homo sapiens protein inhibitor of activated ST; 38192_at Cluster Incl. AB014543:Homo sapiens mRNA for KIAA0643 protein, partial; 40026_g_at Cluster Incl. AF052186:Homo sapiens clone 24431 mRNA sequence /cds=UN; 33720_at Cluster Incl. L48692:Homo sapiens (clone p5-23-3) mRNA /cds=UNKNOWN /gb; 34667_at Cluster Incl. U15306:Human cysteine-rich sequence-specific DNA-binding; 36504_at Cluster Incl. AF097441:Homo sapiens phenylalanine-tRNA synthetase (FARS; 37178_at Cluster Incl. M74089:Human TB1 gene mRNA, 3 end /cds=(0,1305) /gb=M740; 39012_g_at Cluster Incl. X99906:Homo sapiens mRNA for alpha endosulfine /cds=(12; 40132_g_at Cluster Incl. D89937:Homo sapiens mRNA for follistatin-related protei; 40823_s_at Cluster Incl. U85430:Human transcription factor NFATx4 mRNA, complete; 32812_at Cluster Incl. AB029025:Homo sapiens mRNA for KIAA1102 protein, partial; 33932_at Cluster Incl. X17644:Human GST1-Hs mRNA for GTP-binding protein /cds=(6; 38390_at Cluster Incl. Z34975:H. sapiens LDLC mRNA /cds=(95,2311) /gb=Z34975 /gi=; 33180_at Cluster Incl. U68111:Human protein phosphatase inhibitor 2 (PPP1R2) gen; 2093_s_at J04977 /FEATURE=mRNA /DEFINITION=HUMKUANT Human Ku autoimmune antigen; 1550_at U19796 /FEATURE= /DEFINITION=HSU19796 Human melanoma antigen p15 mRNA, c; 1206_at X66364 /FEATURE=cds /DEFINITION=HSSTHPKE H. sapiens mRNA PSSALRE for seri; 913_at M21535 /FEATURE= /DEFINITION=HUMERG11 Human erg protein (ets-related gene.

Metagene 431; 37785_at Cluster Incl. U69563:U69563 Homo sapiens cDNA /clone=25050 /gb=U69563 /; 41542_at Cluster Incl. AF062346:Homo sapiens zinc finger protein 216 splice vari; 376_at AB000220 /FEATURE= /DEFINITION=AB000220 Homo sapiens mRNA for semaphorin.

Metagene 432; 36233_at Cluster Incl. AF091242:Homo sapiens ATP sulfuerylase/APS kinase 2 mRNA,; 32075_at Cluster Incl. D89859:Homo sapiens mRNA for zinc finger 5 protein, compl; 33761_s_at Cluster Incl. AB007962:Homo sapiens mRNA, chromosome 1 specific trans; 36042_at Cluster Incl. X75958:H. sapiens trkB mRNA for protein-tyrosine kinase /c; 41735_at Cluster Incl. AI808958:wf67a09.x1 Homo sapiens cDNA, 3 end /clone=IMAG; 1929_at U83508 /FEATURE= /DEFINITION=HSU83508 Human angiopoietin-1 mRNA, complet; 1867_at AF005775 /FEATURE= /DEFINITION=AF005775 Homo sapiens caspase-like apopto; 1115_at M25897 /FEATURE=mRNA /DEFINITION=HUMPF4A Human platelet factor 4 (PF4) m.

Metagene 433; 34169_s_at Cluster Incl. U57627:Human fetal brain oculocerebrorenal syndrome (OC; 35074_at Cluster Incl. AF004715:Homo sapiens jerky gene product homolog mRNA, co; 36792_at Cluster Incl. Z24727:H. sapiens tropomyosin isoform mRNA, complete CDS /; 37828_at Cluster Incl. AL050064:Homo sapiens mRNA; cDNA DKFZp566L033 (from clone; 38139_at Cluster Incl. AF017445:Homo sapiens GDP-L-fucose pyrophosphorylase (GFP; 38568_at Cluster Incl. U82939:Homo sapiens p53 binding protein mRNA, complete cd; 40338_at Cluster Incl. U52827:Homo sapiens Cri-du-chat region mRNA, clone NIBB11; 40701_at Cluster Incl. U75362:Homo sapiens isopeptidase T-3 (ISOT-3) mRNA, compl; 41366_at Cluster Incl. AB023219:Homo sapiens mRNA for KIAA1002 protein, complete; 41421_at Cluster Incl. AB020716:Homo sapiens mRNA for KIAA0909 protein, partial; 41612_at Cluster Incl. AB007872:Homo sapiens KIAA0412 mRNA, partial cds /cds=(36; 31851_at Cluster Incl. AJ224819:Homo sapiens mRNA for candidate tumor suppressor; 31854_at Cluster Incl. AF035582:Homo sapiens CASK mRNA, complete cds /cds=(15,27; 32087_at Cluster Incl. M65217:Human heat shock factor 2 (HSF2) mRNA, complete cd; 32119_at Cluster Incl. AL049423:Homo sapiens mRNA; cDNA DKFZp586B211 (from clone; 32730_at Cluster Incl. AL080059:Homo sapiens mRNA; cDNA DKFZp564H142 (from clone; 33241_at Cluster Incl. AB014526:Homo sapiens mRNA for KIAA0626 protein, complete; 33809_at Cluster Incl. AL049933:Homo sapiens mRNA; cDNA DKFZp564K1216 (from clon; 34249_at Cluster Incl. AF084535:Homo sapiens laforin (EPM2A) mRNA, partial cds /; 34745_at Cluster Incl. AF070570:Homo sapiens clone 24473 mRNA sequence /cds=UNKN; 35234_at Cluster Incl. D50406:Homo sapiens ST15 mRNA, complete cds /cds=(109,302; 35720_at Cluster Incl. AB020700:Homo sapiens mRNA for KIAA0893 protein, complete; 36002_at Cluster Incl. AB023229:Homo sapiens mRNA for KIAA1012 protein, complete; 36089_at Cluster Incl. AB023183:Homo sapiens mRNA for KIAA0966 protein, complete; 36827_at Cluster Incl. AF020762:Homo sapiens clone 1400 unknown protein mRNA, pa; 36905_at Cluster Incl. AB009356:Homo sapiens mRNA for TGF-beta activated kinase; 36909_at Cluster Incl. X62048:H. sapiens Wee1 hu gene /cds=(170,2110) /gb=X62048; 37535_at Cluster Incl. M27691:Human transactivator protein (CREB) mRNA, complete; 37581_at Cluster Incl. X92972:H. sapiens mRNA for protein phosphatase 6 /cds=(21,; 37891_at Cluster Incl. U79272:Human clone 23720 mRNA sequence /cds=UNKNOWN /gb=U; 38674_at Cluster Incl. AA115140:zl10d12.r1 Homo sapiens cDNA, 5 end /clone=IMAG; 38698_at Cluster Incl. AL080070:Homo sapiens mRNA; cDNA DKFZp564M112 (from clone; 39376_at Cluster Incl. AB014530:Homo sapiens mRNA for KIAA0630 protein, partial; 39692_at Cluster Incl. AL080209:Homo sapiens mRNA; cDNA DKFZp586F2423 (from clon; 39707_at Cluster Incl. AB014547:Homo sapiens mRNA for KIAA0647 protein, partial; 40105_at Cluster Incl. M65131:Human methylmalonyl-CoA mutase (MCM) mRNA, complet; 40438_at Cluster Incl. D87930:Homo sapiens mRNA for myosin phosphatase target su; 40488_at Cluster Incl. M18533:Homo sapiens dystrophin (DMD) mRNA, complete cds /; 40492_at Cluster Incl. AB020633:Homo sapiens mRNA for KIAA0826 protein, partial; 40780_at Cluster Incl. AF016507:Homo sapiens C-terminal binding protein 2 mRNA,; 40853_at Cluster Incl. AI478147:tm34f06.x1 Homo sapiens cDNA, 3 end /clone=IMAG; 40859_at Cluster Incl. AI561196:tq27a01.x1 Homo sapiens cDNA, 3 end /clone=IMAG; 32160_at Cluster Incl. U76247:Human hSIAH1 mRNA, complete cds /cds=(186,1034) /g; 32854_at Cluster Incl. AB014596:Homo sapiens mRNA for KIAA0696 protein, partial; 33340_at Cluster Incl. AB007898:Homo sapiens KIAA0438 mRNA, complete cds /cds=(1; 34335_at Cluster Incl. AI765533:wi81c01.x1 Homo sapiens cDNA, 3 end /clone=IMAG; 34344_at Cluster Incl. AF044195:Homo sapiens IkappaB kinase complex associated p; 35319_at Cluster Incl. U25435:Human transcriptional repressor (CTCF) mRNA, compl; 35321_at Cluster Incl. AB004884:Homo sapiens mRNA for PKU-alpha, partial cds /cd; 35843_at Cluster Incl. L40402:Homo sapiens (clone Zap2) mRNA fragment /cds=UNKNO; 36117_at Cluster Incl. L13616:Human focal adhesion kinase (FAK) mRNA, complete c; 36192_at Cluster Incl. D83777:Human mRNA for KIAA0193 gene, complete cds /cds=(3; 36198_at Cluster Incl. D13641:Human mRNA for KIAA0016 gene, complete cds /cds=(1; 36587_at Cluster Incl. Z11692:H. sapiens mRNA for elongation factor 2 /cds=(0,257; 36934_at Cluster Incl. AL035447:Human DNA sequence from clone 1183I21 on chromos; 36944_f_at Cluster Incl. U72621:Human LOT1 mRNA, complete cds /cds=(657,2048) /g; 36946_at Cluster Incl. D86550:Human mRNA for serine/threonine protein kinase, co; 37662_at Cluster Incl. AI701164:we10g07.x1 Homo sapiens cDNA, 3 end tclone=IMAG; 37736_at Cluster Incl. D13892:Human mRNA for carboxylmethyltransferase, complet; 38011_at Cluster Incl. AB006572:Homo sapiens RMP mRNA for RPB5 meidating protein; 38075_at Cluster Incl. X68194:H. sapiens h-Sp1 mRNA /cds=(33,812) /gb=X68194 /gi=; 38821_at Cluster Incl. AJ002030:Homo sapiens mRNA for putative progesterone bind; 39132_at Cluster Incl. AB010882:Homo sapiens mRNA for hSNF2H, complete cds /cds=; 39150_at Cluster Incl. U69559:U69559 Homo sapiens cDNA /clone=26077 /gb=U69559 /; 39509_at Cluster Incl. AI692348:wd85g12.x1 Homo sapiens cDNA, 3 end /clone=IMAG; 40899_at Cluster Incl. Y00503:Human mRNA for keratin 19 /cds=(32,1234) /gb=Y0050; 41343_at Cluster Incl. Y16521:Homo sapiens mRNA for CDS2 protein /cds=(258,1595); 32530_at Cluster Incl. X56468:Human mRNA for 14.3.3 protein, a protein kinase re; 32569_at Cluster Incl. L13385:Homo sapiens (clone 71) Miller-Dieker lissencephaly; 1940_at M54968 /FEATURE= /DEFINITION=HUMKRASM Human K-ras oncogene protein mRNA,; 1530_g_at U50534 /FEATURE= /DEFINITION=HSU50534 Human BRCA2 region, mRNA sequenc; 851_s_at S62539 /FEATURE= /DEFINITION=S62539 insulin receptor substrate-1 [human; 755_at D26070 /FEATURE=mRNA /DEFINITION=HUMINSP3R1 Human mRNA for type 1 inosito; 160_at U43899 /FEATURE= /DEFINITION=HSU43899 Human signal transducing adaptor mo.

Metagene 434; 31525_s_at Cluster Incl. J00153:Human alpha globin gene cluster on chromosome 16; 31687_f_at Cluster Incl. M25079:Human sickle cell beta-globin mRNA, complete cds; 32052_at Cluster Incl. L48215:Homo sapiens beta-globin (HBB) gene, with a to c a; 32799_at Cluster Incl. AF023268:Homo sapiens clk2 kinase (CLK2), propin1, cote1,; 215_g_at M97676 /FEATURE= /DEFINITION=HUMHOX7 Homo sapiens (region 7) homeobox P.

Metagene 435; 35092_at Cluster Incl. AF032387:Homo sapiens snRNA activating protein complex 19; 38935_at Cluster Incl. X72790:Human endogenous retrovirus mRNA for ORF /cds=(524; 38337_at Cluster Incl. U62392:Homo sapiens zinc finger protein mRNA, complete cd; 40221_at Cluster Incl. L39059:Homo sapiens transcription factor SL1 mRNA, comple; 1468_at U12595 /FEATURE= /DEFINITION=HSU12595 Human tumor necrosis factor type 1; 576_at M93718 /FEATURE= /DEFINITION=HUMNIOXSYN Human nitric oxide synthase mRNA,.

Metagene 436; 35948_at Cluster Incl. D25303:Human mRNA for integrin alpha subunit, complete cd; 39324_at Cluster Incl. AL050078:Homo sapiens mRNA; cDNA DKFZp566G0746 (from clon; 40673_at Cluster Incl. U12778:Human acyl-CoA dehydrogenase mRNA, complete cds /c; 40731_at Cluster Incl. L07515:Human heterochromatin protein homologue (HP1) mRNA; 32142_at Cluster Incl. U26424:Human Ste20-like kinase (MST2) mRNA, complete cds; 34288_at Cluster Incl. U67784:Human orphan G protein-coupled receptor (RDC1) mRN; 35246_at Cluster Incl. U18934:Human receptor tyrosine kinase (DTK) mRNA, complet; 37532_at Cluster Incl. M91432:Human medium-chain acyl-CoA dehydrogenase (MCAD) g; 39009_at Cluster Incl. N98670:yy66d08.r1 Homo sapiens cDNA, 5 end /clone=IMAGE-; 39690_at Cluster Incl. AF002282:Homo sapiens alpha-actinin-2 associated LIM prot; 40876_at Cluster Incl. U31525:Human glycogenin mRNA, complete cds /cds=(127,1128; 41188_at Cluster Incl. W28186:43c2 Homo sapiens cDNA /gb=W28186 /gi=1308134 /ug=; 33456_at Cluster Incl. U24166:Homo sapiens EB1 mRNA, complete cds /cds=(64,870); 33876_at Cluster Incl. AL050107:Homo sapiens mRNA; cDNA DKFZp586I1419 (from clon; 34809_at Cluster Incl. H53921:yq87g03.r1 Homo sapiens cDNA, 5 end /clone=IMAGE-; 35837_at Cluster Incl. AJ224677:Homo sapiens mRNA for scrapie responsive protein; 36149_at Cluster Incl. D78014:Homo sapiens mRNA for dihydropyrimidinase related; 36211_at Cluster Incl. D87461:Human mRNA for KIAA0271 gene, complete cds /cds=(1; 36650_at Cluster Incl. D13639:Human mRNA for KIAK0002 gene, complete cds /cds=(2; 37035_at Cluster Incl. AI557272:PT2.1_{—}15_G02.r Homo sapiens cDNA, 3 end /clone; 37318_at Cluster Incl. X81625:H. sapiens mRNA for C1I protein /cds=(135,1448) /gb; 37347_at Cluster Incl. AA926959:om68h08.s1 Homo sapiens cDNA, 3 end /clone=IMAG; 38033_at Cluster Incl. AL049934:Homo sapiens mRNA; cDNA DKFZp564M1416 (from clon; 38802_at Cluster Incl. Y12711:H. sapiens mRNA for putative progesterone binding p; 39147_g_at Cluster Incl. U72936:Human putative DNA dependent ATPase and helicase; 40213_at Cluster Incl. M88163:Human global transcription activator homologous se; 1845_at L36870 /FEATURE=mRNA /DEFINITION=HUMMKK4A Homo sapiens MAP kinase kinase; 1448_at D00762 /FEATURE= /DEFINITION=HUMPSC8 Human mRNA for proteasome subunit H; 949_s_at D78275 /FEATURE= /DEFINITION=D78275 Homo sapiens mRNA for proteasome su; 539_at S59184 /FEATURE= /DEFINITION=S59184 RYK=related to receptor tyrosine kina.

Metagene 437; 36446_s_at Cluster Incl. L24521:Human transformation-related protein mRNA, 3 en; 36694_at Cluster Incl. AF043472:Homo sapiens Shab-related delayed-rectifier K+ c; 38170_at Cluster Incl. AF070595:Homo sapiens clone 24583 mRNA sequence /cds=UNKN; 38856_at Cluster Incl. AL109724:Homo sapiens mRNA full length insert cDNA clone; 40000_f_at Cluster Incl. AB023203:Homo sapiens mRNA for KIAA0986 protein, partia; 41419_at Cluster Incl. AL080142:Homo sapiens mRNA; cDNA DKFZp434N103 (from clone; 41655_at Cluster Incl. AL034399:dA191P20.2 (novel Fibronectin type III domain co; 31792_at Cluster Incl. M20560:Human lipocortin-III mRNA, complete cds /cds=(46,1; 32026_s_at Cluster Incl. AB002311:Human mRNA for KIAA0313 gene, complete cds /cd; 33222_at Cluster Incl. AB017365:Homo sapiens mRNA for frizzled-7, complete cds /; 33716_at Cluster Incl. N95443:zb81c12.s1 Homo sapiens cDNA, 3 end /clone=IMAGE-; 33759_at Cluster Incl. X04327:Human erythrocyte 2,3-bisphosphoglycerate mutase m; 34654_at Cluster Incl. AJ224979:Homo sapiens mRNA for MTMR1 protein /cds=(0,1990; 35237_at Cluster Incl. AL034452:Human DNA sequence from clone 682J15 on chromoso; 36542_at Cluster Incl. AF030409:Homo sapiens sodium-hydrogen exchanger 6 (NHE-6); 37273_at Cluster Incl. AF007153:Homo sapiens clone 23736 mRNA sequence /cds=UNKN; 37619_at Cluster Incl. D42084:Human mRNA for KIAA0094 gene, partial cds /cds=(0,; 38279_at Cluster Incl. D90150:Human Gx-alpha gene /cds=(619,1686) /gb=D90150 /gi; 38980_at Cluster Incl. AB018276:Homo sapiens mRNA for KIAA0733 protein, partial; 39410_at Cluster Incl. AB007860:Homo sapiens KIAA0400 mRNA, complete cds /cds=(3; 41128_at Cluster Incl. AF070537:Homo sapiens clone 24606 mRNA sequence /cds=UNKN; 41747_s_at Cluster Incl. U49020:Human myocyte-specific enhancer factor 2A (MEF2A; 32805_at Cluster Incl. U05861:Human hepatic dihydrodiol dehydrogenase gene /cds=; 35310_at Cluster Incl. D45288:HUMHG2121 Homo sapiens cDNA /gb=D45288 /gi=1136684; 35371_at Cluster Incl. M83822:Human beige-like protein (BGL) mRNA, partial cds /; 36636_at Cluster Incl. M12267:Human ornithine aminotransferase mRNA, complete cd; 38385_at Cluster Incl. S65738:actin depolymerizing factor [human, fetal brain, m; 39136_at Cluster Incl. AB017642:Homo sapiens mRNA for oxidative-stress responsiv; 40220_at Cluster Incl. AB021179:Homo sapiens mRNA for HEXIM1 protein, complete c; 41536_at Cluster Incl. AL022726:Human DNA sequence from clone 625H18 on chromoso; 1593_at J04513 /FEATURE=mRNA /DEFINITION=HUMGFB Human basic fibroblast growth fa; 1495_at M34057 /FEATURE= /DEFINITION=HUMTGFB1B Human transforming growth factor-; 857_at S87759 /FEATURE= /DEFINITION=S87759 protein phosphatase 2C alpha [human,; 160043_at X66087 /FEATURE=cds /DEFINITION=HSAMYB2 H. sapiens a-myb mRNA /NOTE=rep.

Metagene 438; 34949_at Cluster Incl. AB028971:Homo sapiens mRNA for KIAA1048 protein, complete; 37085_g_at Cluster Incl. AF088219:Homo sapiens CC chemokine gene cluster, comple; 38733_at Cluster Incl. M30938:Human Ku (p70 /p80) subunit mRNA, complete cds /cds.

Metagene 439; 31936_s_at Cluster Incl. AB007890:Homo sapiens KIAA0430 mRNA, complete cds /cds=; 33666_at Cluster Incl. M16342:Human nuclear ribonucleoprotein particle (hnRNP)C; 34987_s_at Cluster Incl. X79536:H. sapiens mRNA for hnRNPcore protein A1 /cds=(26; 38908_s_at Cluster Incl. AL096744:Homo sapiens mRNA; cDNA DKFZp566H033 (from clo; 41457_at Cluster Incl. AB007883:Homo sapiens KIAA0423 mRNA, partial cds /cds=(0,; 41669_at Cluster Incl. D83776:Human mRNA for KIAA0191 gene, partial cds /cds=(0,; 32085_at Cluster Incl. AB023198:Homo sapiens mRNA for KIAA0981 protein, partial; 32654_g_at Cluster Incl. AW020536:df11b12.y1 Homo sapiens cDNA, 5 end /clone=IM; 33264_at Cluster Incl. X89602:H. sapiens mRNA for rTS beta protein /cds=(17,1267); 33774_at Cluster Incl. X98172:H. sapiens mRNA for MACH-alpha-1 protein /cds=(291,; 34225_at Cluster Incl. AF101434:Homo sapiens Wolf-Hirschhorn syndrome candidate; 34269_at Cluster Incl. AL050102:Homo sapiens mRNA; cDNA DKFZp586F1019 (from clon; 35163_at Cluster Incl. AB028964:Homo sapiens mRNA for KIAA1041 protein, complete; 35614_at Cluster Incl. AB012124:Homo sapiens TCFL5 mRNA for transcription factor; 36032_at Cluster Incl. AL031427:dJ167A19.3 (novel protein) /cds=(123,557) /gb=AL; 36046_at Cluster Incl. AL050144:Homo sapiens mRNA; cDNA DKFZp586C1620 (from clon; 36514_at Cluster Incl. U66469:Human cell growth regulator CGR19 mRNA, complete c; 36551_at Cluster Incl. AL049382:Homo sapiens mRNA; cDNA DKFZp586K1318 (from clon; 36845_at Cluster Incl. D50926:Human mRNA for KIAA0136 gene, partial cds /cds=(0,; 36910_at Cluster Incl. L36870:Homo sapiens MAP kinase kinase 4 (MKK4) mRNA, comp; 37197_s_at Cluster Incl. AL050006:Homo sapiens mRNA; cDNA DKFZp564A033 (from clo; 37212_at Cluster Incl. D28588:Human mRNA for KIAA0048 gene, complete cds /cds=(3; 37895_at Cluster Incl. D87969:Homo sapiens mRNA for CMP-sialic acid transporter,; 38344_at Cluster Incl. R40666:yf79c10.s1 Homo sapiens cDNA, 3 end /clone=IMAGE-; 38357_at Cluster Incl. AL049321:Homo sapiens mRNA; cDNA DKFZp564D156 (from clone; 38365_at Cluster Incl. AF026086:Homo sapiens peroxisome biogenesis disorder prot; 39415_at Cluster Incl. X72727:H. sapiens tunp mRNA for transformation upregulated; 40091_at Cluster Incl. U00115:Human zinc-finger protein (bcl-6) mRNA, complete c; 40864_at Cluster Incl. D25274:Homo sapiens mRNA, clone-PO2ST9 /cds=UNKNOWN /gb=D; 41129_at Cluster Incl. D26067:Human mRNA for KIAA0033 gene, partial cds /cds=(0,; 41224_at Cluster Incl. AB018331:Homo sapiens mRNA for KIAA0788 protein, partial; 41784_at Cluster Incl. AL080186:Homo sapiens mRNA; cDNA DKFZp564B0769 (from clon; 32165_at Cluster Incl. L41887:Homo sapiens splicing factor, arginine/serine-rich; 32183_at Cluster Incl. M74002:Human arginine-rich nuclear protein mRNA, complete; 32255_i_at Cluster Incl. U40705:Homo sapiens telomeric repeat binding factor (TR; 32259_at Cluster Incl. AB002386:Human mRNA for KIAA0388 gene, complete cds /cds=; 32857_at Cluster Incl. L13858:Human guanine nucleotide exchange factor mRNA, com; 33348_at Cluster Incl. M80627:Human HEB helix-loop-helix protein (HEB) mRNA, com; 33373_at Cluster Incl. AL049951:Homo sapiens mRNA; cDNA DKFZp564O0122 (from clon; 33835_at Cluster Incl. AB018264:Homo sapiens mRNA for KIAA0721 protein, partial; 34330_at Cluster Incl. AB007618:Homo sapiens mRNA for COX7RP, complete cds /cds=; 34804_at Cluster Incl. AL049246:Homo sapiens mRNA; cDNA DKFZp564C053 (from clone; 34877_at Cluster Incl. AL039831:DKFZp434D1112_s1 Homo sapiens cDNA, 3 end /clon; 34879_at Cluster Incl. AF007875:Homo sapiens dolichol monophosphate mannose synt; 35283_at Cluster Incl. H05692:yl76b12.s1 Homo sapiens cDNA, 3 end /clone=IMAGE-; 35762_at Cluster Incl. AB007952:Homo sapiens mRNA for KIAA0483 protein, partial; 35829_at Cluster Incl. AL080181:Homo sapiens mRNA; cDNA DKFZp434O111 (from clone; 36633_at Cluster Incl. AA114830:zk88e06.s1 Homo sapiens cDNA, 3 end /clone=IMAG; 36635_at Cluster Incl. AB023173:Homo sapiens mRNA for KIAA0956 protein, partial; 36648_at Cluster Incl. AF031383:Homo sapiens hMed7 (MED7) mRNA, complete cds /cd; 36660_at Cluster Incl. AF000231:Homo sapiens rab11a GTPase mRNA, complete cds /c; 36690_at Cluster Incl. M10901:Human glucocorticoid receptor alpha mRNA, complete; 37694_at Cluster Incl. D87685:Human mRNA for KIAA0244 gene, partial cds /cds=(0,; 37703_at Cluster Incl. Y08201:Homo sapiens mRNA for rab geranylgeranyl transfera; 37732_at Cluster Incl. AL049940:Homo sapiens mRNA; cDNA DKFZp564E1922 (from clon; 38050_at Cluster Incl. D79986:Human mRNA for KIAA0164 gene, complete cds /cds=(2; 38818_at Cluster Incl. Y08685:H. sapiens mRNA for serine palmitoyltransferase, su; 40605_at Cluster Incl. AA524345:ng43a06.s1 Homo sapiens cDNA, 3 end /clone=IMAG; 40885_s_at Cluster Incl. N30151:yx81f01.s1 Homo sapiens cDNA, 3 end /clone=IMAG; 40988_at Cluster Incl. AJ132637:Homo sapiens mRNA for ATP-dependent metalloprote; 41283_at Cluster Incl. AF052131:Homo sapiens clone 23930 mRNA sequence /cds=UNKN; 41547_at Cluster Incl. AF047472:Homo sapiens spleen mitotic checkpoint BUB3 (BUB; 41573_at Cluster Incl. X68560:H. sapiens SPR-2 mRNA for GT box binding protein /c; 1527_s_at U50527 /FEATURE= /DEFINITION=HSU50527 Human BRCA2 region, mRNA sequenc; 1512_at D86550 /FEATURE= /DEFINITION=D86550 Human mRNA for serine/threonine prot; 1030_s_at U07806 /FEATURE= /DEFINITION=HSU07806 Human camptothecin resistant clo; 582_g_at M29960 /FEATURE=mRNA /DEFINITION=HUMTR211 Human steroid receptor (TR2-1; 192_at U18062 /FEATURE= /DEFINITION=HSU18062 Human TFIID subunit TAFII55 (TAFII5.

Metagene 440; 33948_at Cluster Incl. U34587:Human corticotropin-releasing factor receptor 2 mR; 35909_at Cluster Incl. Z50194:H. sapiens mRNA for PQ-rich protein /cds=(159,1361); 41447_at Cluster Incl. AB023207:Homo sapiens mRNA for KIAA0990 protein, complete; 32066_g_at Cluster Incl. S68134:CREM=cyclic AMP-responsive element modulator bet; 32067_at Cluster Incl. S68271:CREM=cyclic AMP-responsive element modulator [huma; 33305_at Cluster Incl. M93056:Human mononcyte/neutrophil elastase inhibitor mRNA; 33705_at Cluster Incl. L20971:Human phosphodiesterase mRNA, complete cds /cds=(7; 33803_at Cluster Incl. J02973:Human thrombomodulin gene, complete cds /cds=(541,; 37985_at Cluster Incl. L37747:Homo sapiens lamin B1 gene /cds=(340,2100) /gb=L37; 38326_at Cluster Incl. M69199:Human GOS2 protein gene, complete cds /cds=(160,47; 40790_at Cluster Incl. AB004066:Homo sapiens mRNA for DEC1, complete cds /cds=(1; 32186_at Cluster Incl. M80244:Human E16 mRNA, complete cds /cds=(310,1035) /gb=M; 33428_s_at Cluster Incl. AF034957:Homo sapiens secreted T cell activation protei; 33849_at Cluster Incl. U02020:Human pre-B cell enhancing factor (PBEF) mRNA, com; 34375_at Cluster Incl. M28225:Human JE gene encoding a monocyte secretory protei; 34777_at Cluster Incl. D14874:Homo sapiens mRNA for adrenomedullin precursor, co; 36100_at Cluster Incl. AF022375:Homo sapiens vascular endothelial growth factor; 38740_at Cluster Incl. X79067:H. sapiens ERF-1 mRNA 3 end /cds=UNKNOWN /gb=X7906; 40968_at Cluster Incl. AB004904:Homo sapiens mRNA for STAT induced STAT inhibito; 1953_at AF024710 /FEATURE= /DEFINITION=AF024710 Homo sapiens vascular endothelia; 1936_s_at Proto-Oncogene C-Myc, Alt. Splice 3, Orf 114; 1519_at J04102 /FEATURE= /DEFINITION=HUMETS2A Human erythroblastosis virus oncog; 1372_at M31165 /FEATURE=mRNA /DEFINITION=HUMTSG6A Human tumor necrosis factor-in; 1237_at S81914 /FEATURE= /DEFINITION=S81914 IEX-1=radiation-inducible immediate-; 1069_at U04636 /FEATURE=mRNA /DEFINITION=HSU04636 Human cyclooxygenase-2 (hCox-2; 874_at M26683 /FEATURE= /DEFINITION=HUMIFNIND Human interferon gamma treatment i; 875_g_at M26683 /FEATURE= /DEFINITION=HUMIFNIND Human interferon gamma treatment; 669_s_at L05072 /FEATURE=expaned-cds /DEFINITION=HUMIFNRF1A Homo sapiens interfe.

Metagene 441; 31931_f_at Cluster Incl. AI632247:tt20f10.x1 Homo sapiens cDNA, 3 end /clone=IM; 32474_at Cluster Incl. X96744:H. sapiens PAX7 gene, exon 1 (and joined CDS) /cds=; 33037_at Cluster Incl. AL022165:dJ71L16.4 (putative Chondroitin 6-Sulfotransfera; 34901_at Cluster Incl. AF079564:Homo sapiens ubiquitin-specific protease UBP41 (; 34903_at Cluster Incl. AI017382:ou92e06.x1 Homo sapiens cDNA, 3 end /clone=IMAG; 40033_at Cluster Incl. AL022328:Human DNA sequence from clone 402G11 on chromoso; 40360_at Cluster Incl. X12458:Human P3 gene /cds=(494,1927) /gb=X12458 /gi=35187; 32101_at Cluster Incl. AA112483:zn69a06.s1 Homo sapiens cDNA, 3 end /clone=IMAG; 36522_at Cluster Incl. AB014516:Homo sapiens mRNA for KIAA0616 protein, partial; 38282_at Cluster Incl. U41767:Human metargidin precursor mRNA, complete cds /cds; 37368_at Cluster Incl. AA292277:zt51a05.s1 Homo sapiens cDNA, 3 end /clone=IMAG; 33204_at Cluster Incl. AI080288:oz51f04.x1 Homo sapiens cDNA, 3 end /clone=IMAG; 1592_at J04088 /FEATURE= /DEFINITION=HUMTOPII Human DNA topoisomerase II (top2); 1263_at M20137 /FEATURE=mRNA /DEFINITION=HUMIL3A Human interleukin 3 (IL-3) mRNA.

Metagene 442; 31508_at Cluster Incl. S73591:brain-expressed HHCPA78 homolog [human, HL-60 acut; 34110_g_at Cluster Incl. AF010310:Homo sapiens p53 induced protein mRNA, partial; 41638_at Cluster Incl. D38552:Human mRNA for KIAA0073 gene, partial cds /cds=(0,; 33325_at Cluster Incl. W26667:11a1 Homo sapiens cDNA /gb=W26667 /gi=1305733 /ug=; 34695_at Cluster Incl. AI816724:wj43c06.x1 Homo sapiens cDNA, 3 end /clone=IMAG; 34740_at Cluster Incl. AF032886:Homo sapiens forkhead protein (FKHRL1) mRNA, com; 36906_at Cluster Incl. U73304:Human CB1 cannabinoid receptor (CNR1) gene, comple; 37909_at Cluster Incl. L34155:Homo sapiens laminin-related protein (LamA3) mRNA,; 38653_at Cluster Incl. D11428:Homo sapiens mRNA for PMP-22(PAS-II/SR13 /Gas-3), c; 39053_at Cluster Incl. AF016370:Homo sapiens U4 /U6 small nuclear ribonucleoprote; 39725_at Cluster Incl. L10910:Homo sapiens splicing factor (CC1.3) mRNA, complet; 40103_at Cluster Incl. X51521:Human mRNA for ezrin /cds=(117,1877) /gb=X51521 /g; 40841_at Cluster Incl. AF049910:Homo sapiens TACC1 (TACC1) mRNA, complete cds /c; 32804_at Cluster Incl. AF091263:Homo sapiens RNA binding motif protein 5 (RBM5); 35785_at Cluster Incl. W28281:47e7 Homo sapiens cDNA /gb=W28281 /gi=1308436 /ug=; 36629_at Cluster Incl. AI635895:tz82a07.x1 Homo sapiens cDNA, 3 end /clone=IMAG; 36961_at Cluster Incl. AL050286:Homo sapiens mRNA; cDNA DKFZp586A011 (from clone; 39533_at Cluster Incl. D87432:Human mRNA for KIAA0245 gene, complete cds /cds=(2; 39545_at Cluster Incl. U22398:Human Cdk-inhibitor p57KIP2 (KIP2) mRNA, complete; 39827_at Cluster Incl. AA522530:ni38d12.s1 Homo sapiens cDNA, 3 end /clone=IMAG; 40570_at Cluster Incl. AF032885:Homo sapiens forkhead protein (FKHR) mRNA, compl; 1629_s_at Tyrosine Phosphatase 1, Non-Receptor, Alt. Splice 3. Metagene 443; 38858_at Cluster Incl. U04270:Human putative potassium channel subunit (h-erg) m; 39078_at Cluster Incl. U43286:Human selenophosphate synthetase 2 (SPS2) mRNA, co; 40559_at Cluster Incl. AL096727:Homo sapiens mRNA; cDNA DKFZp434B104 (from clone.

Metagene 444; 35756_at Cluster Incl. AF089816:Homo sapiens RGS-GAIP interacting protein GIPC m; 39124_r_at Cluster Incl. X89066:H. sapiens mRNA for TRPC1 protein /cds=(137,2416); 33207_at Cluster Incl. AI095508:qb29a06.x1 Homo sapiens cDNA, 3 end /clone=IMAG.

Metagene 445; 34621_at Cluster Incl. AF019084:Homo sapiens keratin 2e (KRT2E) gene, complete c; 36411_s_at Cluster Incl. U29943:Human ELAV-like neuronal protein-2 Hel-N2 mRNA,; 32971_at Cluster Incl. L27479:Human X123 mRNA, 3 end /cds=(2,739) /gb=L27479 /g; 33516_at Cluster Incl. V00505:Human gene for delta-globin /cds=(50,493) /gb=V005; 37140_s_at Cluster Incl. AF061193:Homo sapiens ectodysplasin-A isoform EDA-E (ED; 39245_at Cluster Incl. U72507:Human 40871 mRNA partial sequence /cds=UNKNOWN /gb; 35999_r_at Cluster Incl. AB018324:Homo sapiens mRNA for KIAA0781 protein, partia; 37593_at Cluster Incl. X14608:Human mRNA for propionyl-CoA carboxylase alpha-cha; 33142_at Cluster Incl. AF034633:Homo sapiens orphan G protein-coupled receptor (; 1683_at X69950 /FEATURE=exon#2 /DEFINITION=HSWT1WIT H. sapiens DNA sequence for W; 1289_at L02321 /FEATURE= /DEFINITION=HUMGSTM5 Human glutathione S-transferase (G.

Metagene 446; 39808_at Cluster Incl. Y12692:Homo sapiens mRNA for WNT11 gene /cds=(123,1187) /; 40532_at Cluster Incl. U75285:Homo sapiens apoptosis inhibitor survivin gene, co; 33109_f_at Cluster Incl. L07335:Homo sapiens (clone 6AR33) HMG box mRNA, 3 end; 33120_at Cluster Incl. AF045229:Homo sapiens regulator of G protein signaling 10; 2082_s_at L08599 /FEATURE= /DEFINITION=HUMUVOECAD Human uvomorulin (E-cadherin); 907_at M13792 /FEATURE=cds /DEFINITION=HUMADAG Human adenosine deaminase (ADA) g; 405_at X52773 /FEATURE=cds /DEFINITION=HSRARLP Human mRNA for retinoic acid rece.

Metagene 447; 35521_at Cluster Incl. AJ130941:Homo sapiens claudin-9 (CLDN9) gene /cds=(0,653); 36406_at Cluster Incl. AA401397:zu68b01.s1 Homo sapiens cDNA, 3 end /clone=IMAG; 33558_at Cluster Incl. Y09445:H. sapiens mRNA for transcription factor TBX5 /cds=; 35375_at Cluster Incl. AJ011311:Homo sapiens mRNA for AP endonuclease XTH2, puta; 36742_at Cluster Incl. U34249:Human putative zinc finger protein (ZNFB7) mRNA, c; 41078_at Cluster Incl. D63484:Human mRNA for KIAA0150 gene, partial cds /cds=(0,; 40494_at Cluster Incl. AF043733:Homo sapiens death effector domain-containing te; 34858_at Cluster Incl. D79998:Human mRNA for KIAA0176 gene, partial cds /cds=(0,; 1552_i_at U22028 /FEATURE=expanded-cds /DEFINITION=HSU22028 Human cytochrome P45; 1091_at M65066 /FEATURE= /DEFINITION=HUMRIB Human cAMP-dependent protein kinase.

Metagene 448; 34647_at Cluster Incl. X52104:Human mRNA for p68 protein /cds=(175,2019) /gb=X52; 33570_at Cluster Incl. U34962:Human transcription factor HCSX (hCsx) mRNA, compl; 34008_at Cluster Incl. AF084465:Homo sapiens Ras-like GTP-binding protein REM mR; 41462_at Cluster Incl. AF065482:Homo sapiens sorting nexin 2 (SNX2) mRNA, comple; 32639_at Cluster Incl. U97198:Homo sapiens CG1 mRNA, complete cds /cds=(219,1490; 36565_at Cluster Incl. X98253:H. sapiens ZNF183 gene /cds=(210,1241) /gb=X98253 /; 39403_at Cluster Incl. AB014578:Homo sapiens mRNA for KIAA0678 protein, partial; 32589_at Cluster Incl. U20979:Human chromatin assembly factor-I p150 subunit mRN; 33149_at Cluster Incl. U73524:Human putative ATP/GTP-binding protein (HEAB) mRNA.

Metagene 449; 36334_at Cluster Incl. L42621:Homo sapiens Ly-9 mRNA, complete cds /cds=(0,1832); 33506_at Cluster Incl. U96919:Homo sapiens inositol polyphosphate 4-phosphatase; 33971_f_at Cluster Incl. U66078:Human DAZLA mRNA, complete cds /cds=(226,1113) /; 34493_at Cluster Incl. AF014794:Homo sapiens TNF related TRAIL receptor (TRAIL-R; 36754_at Cluster Incl. X60435:H. sapiens gene PACAP for pituitary adenylate cycla; 37831_at Cluster Incl. AB011117:Homo sapiens mRNA for KIAA0545 protein, partial; 38521_at Cluster Incl. X59350:H. sapiens mRNA for B cell membrane protein CD22 /c; 36874_at Cluster Incl. M26004:Human CR2 /CD21 /C3d/Epstein-Barr virus receptor mRN; 1201_at D14889 /FEATURE= /DEFINITION=HUMSGBP Human mRNA for small GTP-binding pr.

Metagene 450; 41429_at Cluster Incl. M65254:Protein phosphatase 2A 65 kDa regulatory subunit-b; 36848_r_at Cluster Incl. AC005258:Homo sapiens chromosome 19, cosmid R30783 /cds; 37205_at Cluster Incl. AB020647:Homo sapiens mRNA for KIAA0840 protein, partial; 37890_at Cluster Incl. X69398:H. sapiens mRNA for OA3 antigenic surface determina; 39375_g_at Cluster Incl. AL022325:Homo sapiens DNA sequence from Fosmid 27C3 on; 37664_at Cluster Incl. X80754:Homo sapiens mRNA for GTP-binding protein /cds=(47; 37764_at Cluster Incl. D87328:Homo sapiens mRNA for HCS, complete cds /cds=(1231; 41545_at Cluster Incl. X66365:H. sapiens mRNA PLSTIRE for serine/threonine protei; 1191_s_at AB003102 /FEATURE= /DEFINITION=AB003102 Homo sapiens mRNA for 26S prot.

Metagene 451; 34117_at Cluster Incl. AL080232:Homo sapiens mRNA; cDNA DKFZp586A061 (from clone; 35041_at Cluster Incl. X53655:H. sapiens mRNA for NGF-2 /cds=(43,816) /gb=X53655; 41690_at Cluster Incl. AL049471:Homo sapiens mRNA; cDNA DKFZp586N012 (from clone; 32102_at Cluster Incl. AB018273:Homo sapiens mRNA for KIAA0730 protein, partial; 35186_at Cluster Incl. AJ009770:Homo sapiens mRNA for putative transcription fac; 37218_at Cluster Incl. D64110:Homo sapiens mRNA for ANA, complete cds /cds=(94,8; 37574_at Cluster Incl. L43821:Homo sapiens enhancer of filamentation (HEF1) mRNA; 37934_at Cluster Incl. AL080078:Homo sapiens mRNA; cDNA DKFZp564D1462 (from clon; 38632_at Cluster Incl. AF088982:Homo sapiens heat shock protein hsp40-3 mRNA, co; 40079_at Cluster Incl. AA156240:zl50c12.s1 Homo sapiens cDNA, 3 end /clone=IMAG; 40099_at Cluster Incl. AB014551:Homo sapiens mRNA for KIAA0651 protein, complete; 33924_at Cluster Incl. AB029014:Homo sapiens mRNA for KIAA1091 protein, partial; 34822_at Cluster Incl. U58334:Human Bcl2, p53 binding protein Bbp/53BP2 (BBP/53B; 35291_at Cluster Incl. AL050287:Homo sapiens mRNA; cDNA DKFZp586C021 (from clone; 35335_at Cluster Incl. AB014519:Homo sapiens mRNA for KIAA0619 protein, complete; 37026_at Cluster Incl. AF001461:Homo sapiens Kruppel-like zinc finger protein Zf; 37678_at Cluster Incl. U23070:Human putative transmembrane protein (nma) mRNA, c; 41346_at Cluster Incl. AJ007583:Homo sapiens mRNA for acetylglucosaminyltransfer; 1895_at J04111 /FEATURE=exon#1 /DEFINITION=HUMJUNA Human c-jun proto oncogene (J; 1860_at U58334 /FEATURE= /DEFINITION=HSU58334 Human Bcl2, p53 binding protein Bb; 1801_at U76638 /FEATURE= /DEFINITION=HSU76638 Human BRCA1-associated RING domain; 1562_g_at U27193 /FEATURE= /DEFINITION=HSU27193 Human protein-tyrosine phosphata.

Metagene 452; 32983_at Cluster Incl. U03865:Human adrenergic alpha-1b receptor protein mRNA, c; 33629_at Cluster Incl. AJ001982:Homo sapiens WWp2-like mRNA complete cds /cds=UN; 32312_at Cluster Incl. X06825:Human mRNA for skeletal beta-tropomyosin /cds=(106; 32932_at Cluster Incl. AL050040:Homo sapiens mRNA; cDNA DKFZp566K0524 (from clon; 34016_s_at Cluster Incl. X78338:Synthetic adenovirus transformed human retina ce; 34904_at Cluster Incl. S40369:glutamate receptor subunit-kainate receptor subuni; 36248_at Cluster Incl. AF070572:Homo sapiens clone 24778 unknown mRNA /cds=(0,17; 37133_at Cluster Incl. AF027406:Homo sapiens muscle-specific serine kinase 1 (MS; 38954_at Cluster Incl. Y18423:Homo sapiens VIP2R gene, exons 1-2 (and joined CDS; 32105_f_at Cluster Incl. U66063:Homo sapiens calcium/calmodulin-dependent protei; 37250_at Cluster Incl. AB007191:Homo sapiens mRNA for AMY-1, complete cds /cds=(; 37996_s_at Cluster Incl. L08835:Homo sapiens DMR-N9, partial cds; and myotonic d; 39018_at Cluster Incl. AF026977:Homo sapiens microsomal glutathione S-transferas; 39030_at Cluster Incl. AJ133534:Homo sapiens mRNA for prenylated Rab acceptor 1; 41139_at Cluster Incl. W26633:34b1 Homo sapiens cDNA /gb=W26633 /gi=1307476 /ug=; 41760_at Cluster Incl. AA978033:oq55e04.s1 Homo sapiens cDNA, 3 end /clone=IMAG; 33412_at Cluster Incl. AI535946:vicpro2.D07.r Homo sapiens cDNA, 5 end /clone-e; 34413_at Cluster Incl. AF038203:Homo sapiens clone 23596 mRNA sequence /cds=UNKN; 35361_at Cluster Incl. W28299:44h4 Homo sapiens cDNA /gb=W28299 /gi=1308247 /ug=; 37309_at Cluster Incl. L09159:Homo sapiens RHOA proto-oncogene multi-drug-resist; 38810_at Cluster Incl. AF039241:AF039241 Homo sapiens cDNA /clone=11-67js /gb=AF; 41534_at Cluster Incl. AB006755:Homo sapiens mRNA for PCDH7 (BH-Pcdh)a, complete; 41807_at Cluster Incl. AL040137:DKFZp434D1813_s1 Homo sapiens cDNA, 3 end /clon; 835_at U41745 /FEATURE /DEFINITION=HSU41745 Human PDGF associated protein mRNA,; 650_s_at L07044 /FEATURE= /DEFINITION=HUMCCDPKB Homo sapiens calcium/calmodulin-; 616_s_at M24748 /FEATURE=cds#2 /DEFINITION=HUMTHRA1A Human thyroid hormone recep.

Metagene 453; 36390_at Cluster Incl. U79526:Human orphan G-protein coupled receptor Dez isofor; 33535_at Cluster Incl. U45448:Human P2x1 receptor mRNA, complete cds /cds=(196,1; 37788_at Cluster Incl. AF052115:Homo sapiens clone 23688 mRNA sequence /cds=UNKN; 37855_at Cluster Incl. M95767:Homo sapiens di-N-acetylchitobiase mRNA, complete; 35692_at Cluster Incl. AL080235:Homo sapiens mRNA; cDNA DKFZp586E1621 (from clon; 37562_at Cluster Incl. L11370:Human protocadherin 42 mRNA, complete cds for abbr; 37959_at Cluster Incl. D63876:Human mRNA for KIAA0154 gene, partial cds /cds=(0,; 33366_at Cluster Incl. AL022238:dJ1042K10.2.1 (novel protein with probable rabGA; 34379_at Cluster Incl. AF082657:Homo sapiens Era GTPase A protein (HERA-A) mRNA,; 41789_r_at Cluster Incl. AB014569:Homo sapiens mRNA for KIAA0669 protein, comple; 1852_at X02910 /FEATURE=expanded_cds /DEFINITION=HSTNFA Human gene for tumor nec.

Metagene 454; 32988_at Cluster Incl. Z30643:H. sapiens mRNA for chloride channel (putative) 213; 32877_i_at Cluster Incl. AA524802:nh33h11.s1 Homo sapiens cDNA /clone=IMAGE-9542; 38498_at Cluster Incl. Z99916:Human DNA sequence from clone 221G9 on chromosome; 38661_at Cluster Incl. X75315:H. sapiens seb4B mRNA /cds=(0,693) /gb=X75315 /gi=4; 38089_at Cluster Incl. D63478:Human mRNA for KIAA0144 gene, complete cds /cds=(1.

Metagene 455; 39672_at Cluster Incl. M64322:Human protein tyrosine phosphatase (LPTPase) mRNA,; 34290_f_at Cluster Incl. W29091:56c5 Homo sapiens cDNA /gb=W29091 /gi=1309057 /u.

Metagene 456; 33010_at Cluster Incl. AL008723:dJ90G24.4 (SAAT1 (low affinity sodium glucose co; 34912_at Cluster Incl. AF052941:Homo sapiens DAP-kinase related protein 1 mRNA,; 32092_at Cluster Incl. AB007937:Homo sapiens mRNA for KIAA0468 protein, complete; 34250_at Cluster Incl. AB029033:Homo sapiens mRNA for KIAA1110 protein, partial; 37556_at Cluster Incl. M81637:Human grancalcin mRNA, complete cds /cds=(119,772); 37563_at Cluster Incl. AB007871:Homo sapiens KIAA0411 mRNA, complete cds /cds=(0; 34810_at Cluster Incl. AC004382:Homo sapiens Chromosome 16 BAC clone CIT987SK-A-.

Metagene 457; 31317_r_at Cluster Incl. M21388:Human unproductively rearranged Ig mu-chain mRNA; 31391_at Cluster Incl. AF040723:Homo sapiens neuroan1 mRNA, complete cds /cds=(1; 31393_r_at Cluster Incl. AB011076:Homo sapiens mRNA for UTF1, complete cds /cds=; 31419_r_at Cluster Incl. AF023203:Homo sapiens homeobox protein Og12 (OGL12) mRN; 31426_at Cluster Incl. U34806:Human G protein-coupled receptor (GPR15) gene, com; 31559_at Cluster Incl. U26209:Human renal sodium/dicarboxylate cotransporter (NA; 34166_at Cluster Incl. S80071:hPROT=brain-specific L-proline transporter [human,; 36352_at Cluster Incl. AI275093:ql65c10.x1 Homo sapiens cDNA, 3 end /clone=IMAG; 36359_at Cluster Incl. M34182:Human testis-specific protein kinase gamma-subunit; 32897_at Cluster Incl. AJ237672:Homo sapiens mRNA for methylenetetrahydrofolate; 33470_at Cluster Incl. AF052177:Homo sapiens clone 24510 mRNA sequence /cds=UNKN; 33510_s_at Cluster Incl. U31216:Human metabotropic glutamate receptor 1 beta (mG; 34970_r_at Cluster Incl. AI655458:tt13a03.x1 Homo sapiens cDNA, 3 end /clone=IM; 35853_at Cluster Incl. AL049654:Novel human mRNA similar to mouse gene PICK1 (TR; 36765_at Cluster Incl. AL080154:Homo sapiens mRNA; cDNA DKFZp434I114 (from clone; 36784_at Cluster Incl. J03071:Human growth hormone (GH-1 and GH-2) and chorionic; 37153_at Cluster Incl. AB014573:Homo sapiens mRNA for KIAA0673 protein, partial; 38897_at Cluster Incl. AJ000730:Homo sapiens mRNA for cationic amino acid transp; 39274_at Cluster Incl. X58521:Human mRNA for p62 nucleoporin /cds=(151,1719) /gb; 39667_at Cluster Incl. AF083898:Homo sapiens RNA-binding protein Nova-2 mRNA, co; 40295_at Cluster Incl. AB009288:Homo sapiens mRNA for N-copine, complete cds /cd; 41705_at Cluster Incl. U69198:U69198 Homo sapiens cDNA /clone=c-32h10 /gb=U69198; 34206_at Cluster Incl. AB018325:Homo sapiens mRNA for KIAA0782 protein, partial; 37238_s_at Cluster Incl. AF014118:Homo sapiens membrane-associated kinase (Myt1); 37968_at Cluster Incl. AF031137:Homo sapiens 1C7 precursor, mRNA, alternatively; 38644_at Cluster Incl. U14588:Human paxillin mRNA, complete cds /cds=(74,1747) /; 41181_r_at Cluster Incl. U49785:Human D-dopachrome tautomerase mRNA, complete cd; 41324_g_at Cluster Incl. U90917:Human clone 23641 mRNA sequence /cds=UNKNOWN /gb; 41553_at Cluster Incl. AI738702:wi22b11.x1 Homo sapiens cDNA, 3 end /clone=IMAG; 41566_at Cluster Incl. AI218364:qh21h01.x1 Homo sapiens cDNA, 3 end /clone=IMAG; 2078_s_at M13228 /FEATURE=cds /DEFINITION=HUMNMCY1A Human N-myc oncogene protein; 2032_s_at U07375 /FEATURE=cds /DEFINITION=HSU07375 Human integrin alpha v gene,; 1944_f_at AF001359 /FEATURE= /DEFINITION=AF001359 Homo sapiens DNA mismatch repa; 1442_at X99101 /FEATURE=cds /DEFINITION=HSRNAERB H. sapiens mRNA for estrogen rec; 1134_at L13738 /FEATURE=mRNA /DEFINITION=HUMNRTYKIN Human activated p21cdc42Hs k; 939_at P97 Antigen, Melanoma-Specific; 888_s_at M62302 /FEATURE=cds#1 /DEFINITION=HUMGDF1 Human growth/differentiation; 715_s_at D87002 /FEATURE=cds#4 /DEFINITION=D87002 Homo sapiens immunoglobulin 1a; 632_at L40027 /FEATURE=mRNA /DEFINITION=HUMGLYSYN Homo sapiens glycogen synthase; 344_s_at D13146 /FEATURE=mRNA#1 /DEFINITION=HUM3CNP3 Homo sapiens gene for 2,3; 188_at U09303 /FEATURE= /DEFINITION=HSU09303 Human T cell leukemia LERK-2 (EPLG2.

Metagene 458; 34637_f_at Cluster Incl. M12963:Human class I alcohol dehydrogenase (ADH1) alpha; 36247_f_at Cluster Incl. M12272:Homo sapiens alcohol dehydrogenase class I gamma; 36533_at Cluster Incl. D83402:Homo sapiens gene for prostacyclin synthase /cds=(; 38342_at Cluster Incl. D87076:Human mRNA for KIAA0239 gene, partial cds /cds=(0,; 32545_r_at Cluster Incl. L12535:Human RSU-1 /RSP-1 mRNA, complete cds /cds=(827,1; 32551_at Cluster Incl. U03877:Human extracellular protein (S1-5) mRNA, complete; 32600_at Cluster Incl. AC004382:Homo sapiens Chromosome 16 BAC clone CIT987SK-A-.

Metagene 459; 34151_at Cluster Incl. AL050284:Homo sapiens mRNA; cDNA DKFZp586M1019 (from clon; 32317_s_at Cluster Incl. U34804:Human thermostable phenol sulfotransferase (STP2; 35388_at Cluster Incl. U14755:Human LIM domain transcription factor LIM-1 (hLIM-; 41709_at Cluster Incl. AF034803:Homo sapiens liprin-beta2 mRNA, partial cds /cds; 32138_at Cluster Incl. L07807:Human dynamin mRNA, alternative exons and complete; 37539_at Cluster Incl. AB023176:Homo sapiens mRNA for KIAA0959 protein, partial; 40071_at Cluster Incl. U03688:Human dioxin-inducible cytochrome P450 (CYP1B1) mR; 40436_g_at Cluster Incl. J03592:Human ADP/ATP translocase mRNA, 3 end, clone pH; 32754_at Cluster Incl. X04201:Human skeletal muscle 1.3 kb mRNA for tropomyosin; 34851_at Cluster Incl. AF011468:Homo sapiens serine/threonine kinase (BTAK) mRNA; 38738_at Cluster Incl. X99584:H. sapiens mRNA for SMT3A protein /cds=(94,405) /gb; 40527_at Cluster Incl. AF000571:Homo sapiens kidney and cardiac voltage dependen; 40923_at Cluster Incl. AA290994:zs45d07.r1 Homo sapiens cDNA, 5 end /clone=IMAG; 41798_at Cluster Incl. AJ222801:Homo sapiens mRNA for neutral sphingomyelinase /.

Metagene 460; 32139_at Cluster Incl. Y09538:H. sapiens mRNA for ZNF185 gene /cds=(40,1398) /gb=; 33247_at Cluster Incl. U86782:Human 26S proteasome-associated pad1 homolog (POH1; 36561_at Cluster Incl. X73424:Homo sapiens gene for propionyl-CoA carboxylase a; 38713_at Cluster Incl. Z99716:bK250D10.1 (sterol regulatory element binding tran; 40784_at Cluster Incl. Z69030:H. sapiens mRNA for gamma 1 isoform of 61 kDa regula; 41757_at Cluster Incl. W25933:15b2 Homo sapiens cDNA /gb=W25933 /gi=1306056 /ug=; 32770_at Cluster Incl. AB018298:Homo sapiens mRNA for KIAA0755 protein, complete; 32829_at Cluster Incl. X97544:H. sapiens mRNA for TIM17 preprotein translocase /c; 36199_at Cluster Incl. X76105:H. sapiens DAP-1 mRNA /cds=(159,467) /gb=X76105 /gi; 40567_at Cluster Incl. X01703:Human gene for alpha-tubulin (b alpha 1) /cds=(213; 41516_at Cluster Incl. U95735:Human SNARE protein Ykt6 (YKT6) mRNA, complete cds; 41517_g_at Cluster Incl. U95735:Human SNARE protein Ykt6 (YKT6) mRNA, complete c; 1449_at D00763 /FEATURE= /DEFINITION=HUMPSC9 Human mRNA for proteasome subunit H.

Metagene 461; 31849_at Cluster Incl. AB011136:Homo sapiens mRNA for KIAA0564 protein, partial; 32058_at Cluster Incl. AF070594:Homo sapiens clone 24570 HNK-1 sulfotransferase; 38771_at Cluster Incl. D50405:Human mRNA for RPD3 protein, complete cds /cds=(63; 2065_s_at L22473 /FEATURE= /DEFINITION=HUMBAXA Human Bax alpha mRNA, complete cd; 131_at X83928 /FEATURE=cds /DEFINITION=HSTAFII28 H. sapiens mRNA for transcriptio.

Metagene 462; 40325_at Cluster Incl. AB014460:Homo sapiens TSC2, NTHL1 /NTH1 and SLC9A3R2 /E3KAR; 33232_at Cluster Incl. AI017574:ou23f10.x1 Homo sapiens cDNA, 3 end /clone=IMAG; 33788_at Cluster Incl. AB002390:Human mRNA for KIAA0392 gene, partial cds /cds=(; 36843_at Cluster Incl. AB005666:Homo sapiens mRNA for GTPase-activating protein,; 37543_at Cluster Incl. D25304:Human mRNA for KIAA0006 gene, partial cds /cds=(0,; 37943_at Cluster Incl. AB002319:Human mRNA for KIAA0321 gene, partial cds /cds=(; 38259_at Cluster Incl. AB002559:Homo sapiens mRNA for hunc 18b2, complete cds /cd; 38635_at Cluster Incl. Z69043:H. sapiens mRNA translocon-associated protein delta; 32195_at Cluster Incl. AL049450:Homo sapiens mRNA; cDNA DKFZp586B1922 (from clon; 32806_at Cluster Incl. M36035:Human peripheral benzodiazepine receptor (hpbs) mR; 35282_r_at Cluster Incl. M33680:Human 26-kDa cell surface protein TAPA-1 mRNA, c; 36131_at Cluster Incl. AJ012008:Homo sapiens genes encoding RNCC protein, DDAH p; 36666_at Cluster Incl. M22806:Human prolyl 4-hydroxylase beta-subunit and disulf; 38066_at Cluster Incl. M81600:Human NAD(P)H-quinone oxireductase gene /cds=(111,; 38418_at Cluster Incl. X59798:Human PRAD1 mRNA for cyclin /cds=(147,1034) /gb=X5; 38477_at Cluster Incl. S81752:DPH2L=candidate tumor suppressor gene (ovarian can; 1797_at U40343 /FEATURE= /DEFINITION=HSU40343 Human CDK inhibitor p19INK4d mRNA,; 149_at U90426 /FEATURE= /DEFINITION=HSU90426 Human nuclear RNA helicase, complet.

Metagene 463; 36751_at Cluster Incl. AF035154:Homo sapiens regulator of G-protein signalling 1; 38157_at Cluster Incl. AF059252:Homo sapiens clone 1 HLA class III protein Dom3z; 31823_at Cluster Incl. M74099:Human displacement protein (CCAAT) mRNA /cds=UNKNO; 34700_at Cluster Incl. AF045239:Homo sapiens brain expressed ring finger protein; 34713_at Cluster Incl. AB002357:Human mRNA for KIAA0359 gene, complete cds /cds=; 35644_at Cluster Incl. AB014598:Homo sapiens mRNA for KIAA0698 protein, complete; 38334_g_at Cluster Incl. Y11731:H. sapiens mRNA for DNA glycosylase /cds=(338,137; 39068_at Cluster Incl. L76702:Homo sapiens protein phosphatase 2A B56-delta (PP2; 34869_at Cluster Incl. AJ133766:Homo sapiens mRNA for ZASP protein, partial /cds; 36118_at Cluster Incl. AJ000882:Homo sapiens mRNA for steroid receptor coactivat; 36598_s_at Cluster Incl. L36818:Human (clone 51C-3) 51C protein mRNA, complete c; 1113_at M22489 /FEATURE= /DEFINITION=HUMBMP2A Human bone morphogenetic protein 2; 840_at U47742 /FEATURE= /DEFINITION=HSU47742 Human monocytic leukaemia zinc fing; 278_at L13436 /FEATURE=mRNA /DEFINITION=HUMGUANCYC Homo sapiens guanylate cyclas.

Metagene 464; 32410_at Cluster Incl. X17651:Human Myf-4 mRNA for myogenic determination factor; 32898_at Cluster Incl. U20582:Human actin-like peptide mRNA, partial cds /cds=(2; 33568_at Cluster Incl. U48861:Human beta 4 nicotinic acetylcholine receptor subu; 35412_at Cluster Incl. D13705:Human mRNA for fatty acids omega-hydroxylase (cyto; 36298_at Cluster Incl. L14565:Human peripherin (PRPH) gene exons 1-9, complete c; 38221_at Cluster Incl. AF100153:Homo sapiens connector enhancer of KSR-like prot; 40317_at Cluster Incl. U57352:Human sodium channel 1 (hBNaC1) mRNA, complete cds; 41002_at Cluster Incl. U59299:Homo sapiens putative monocarboxylate transporter; 33699_at Cluster Incl. M18667:Human pepsinogen C gene /cds=(73,1230) /gb=M18667; 35997_g_at Cluster Incl. X98261:H. sapiens mRNA for M-phase phosphoprotein, mpp5; 38309_r_at Cluster Incl. AA805659:nz41b04.s1 Homo sapiens cDNA /clone=IMAGE-1290; 39016_r_at Cluster Incl. L42611:Homo sapiens keratin 6 isoform K6e (KRT6E) mRNA,; 39720_g_at Cluster Incl. X56777:H. sapiens mRNA for ZP3 gene /cds=(30,1148) /gb=X; 34301_r_at Cluster Incl. Z19574:H. sapiens gene for cytokeratin 17 /cds=(64,1362); 40266_at Cluster Incl. AB028959:Homo sapiens mRNA for KIAA1036 protein, complete; 40270_at Cluster Incl. AA969267:on57d12.s1 Homo sapiens cDNA, 3 end /clone=IMAG.

Metagene 465; 40379_at Cluster Incl. J02625:Human cytochrome P-450j mRNA, complete cds /cds=(1; 37240_at Cluster Incl. U18937:Human histidyl-tRNA synthetase homolog (HO3) mRNA,; 36109_at Cluster Incl. J04605:Human prolidase (imidodipeptidase) mRNA, complete.

Metagene 466; 31342_at Cluster Incl. X85019:H. sapiens mRNA for UDP-GalNAc-polypeptide N-acetyl; 31699_at Cluster Incl. S67334:phosphatidylinositol 3-kinase p110 beta isoform=11; 33581_at Cluster Incl. AF023259:Homo sapiens RNA-binding protein (RBMS3) mRNA, c; 37450_r_at Cluster Incl. X04409:Human mRNA for coupling protein G(s) alpha-subun; 37814_g_at Cluster Incl. AL079273:Homo sapiens mRNA full length insert cDNA clon; 33718_at Cluster Incl. AC006128:Homo sapiens chromosome 19, cosmid F20900 /cds=(; 34694_at Cluster Incl. U66618:Human SWI/SNF complex 60 KDa subunit (BAF60b) mRNA; 34742_at Cluster Incl. Z23115:H. sapiens bcl-xL mRNA /cds=(134,835) /gb=Z23115 /g; 39011_at Cluster Incl. X99906:Homo sapiens mRNA for alpha endosulfine /cds=(125,; 39694_at Cluster Incl. W27517:31h6 Homo sapiens cDNA /gb=W27517 /gi=1307321 /ug=; 40847_at Cluster Incl. AB018293:Homo sapiens mRNA for KIAA0750 protein, complete; 33843_g_at Cluster Incl. AC004472:Homo sapiens chromosome 9, P1 clone 11659 /cds; 35339_at Cluster Incl. AI743606:wg51f08.x1 Homo sapiens cDNA, 3 end /clone=IMAG; 38022_s_at Cluster Incl. Z54367:H. sapiens gene for plectin /cds=(0,14054) /gb=Z5; 39537_at Cluster Incl. X98248:H. sapiens mRNA for sortilin /cds=(21,2522) /gb=X98; 39879_s_at Cluster Incl. H16917:ym39e02.r1 Homo sapiens cDNA, 5 end /clone=IMAG; 41291_at Cluster Incl. AC004528:Homo sapiens chromosome 19, cosmid R32184 /cds=(; 41594_at Cluster Incl. M64174:Human protein-tyrosine kinase (JAK1) mRNA, complet; 2041_i_at M14752 /FEATURE= /DEFINITION=HUMABLA Human c-abl gene, complete cds; 2043_s_at M15025 /FEATURE=cds /DEFINITION=HUMBCRABL Human BCR/ABL mRNA (product; 1974_s_at X02469 /FEATURE=cds /DEFINITION=HSP53 Human mRNA for p53 cellular tumo; 1906_at Ras Inhibitor Inf; 1917_at X03484 /FEATURE=cds /DEFINITION=HSRAFR Human mRNA for raf oncogene; 1781_at M25269 /FEATURE= /DEFINITION=HUMELK1A Homo sapiens tyrosine kinase (ELK1; 1764_s_at D85131 /FEATURE= /DEFINITION=D85131 Homo sapiens mRNA for Myc-associat; 1703_g_at S75174 /FEATURE= /DEFINITION=S75174 E2F-4=transcription factor [human,; 1615_at Z23115 /FEATURE=cds /DEFINITION=HSBCLXL H. sapiens bcl-xL mRNA; 1439_s_at X75346 /FEATURE=cds /DEFINITION=HSMAPKAP H. sapiens mRNA for MAP kinase; 1362_s_at M84820 /FEATURE= /DEFINITION=HUMRXRB Human retinoid X receptor beta (R; 919_at Guanine Nucleotide-Binding Protein, Alpha 12; 826_at U50553 /FEATURE= /DEFINITION=HSU50553 Homo sapiens helicase like protein; 716_at D87002 /FEATURE=cds#5 /DEFINITION=D87002 Homo sapiens immunoglobulin lamb; 727_at Ornithine Aminotransferase-Like 3; 463_g_at U70862 /FEATURE= /DEFINITION=HSU70862 Human nuclear factor I B3 mRNA, c; 446_at U89896 /FEATURE= /DEFINITION=HSU89896 Homo sapiens casein kinase I gamma; 424_s_at X66945 /FEATURE=cds /DEFINITION=HSNSAMTK H. sapiens N-sam mRNA for fibro; 368_at Z29083 /FEATURE=cds /DEFINITION=HS5T4OA H. sapiens 5T4 gene for 5T4 Oncofe; 352_at D30036 /FEATURE= /DEFINITION=HUMPITPA Human mRNA for phosphatidylinositol; 111_at Y08200 /FEATURE= /DEFINITION=HSRABGTRA Homo sapiens mRNA for rab geranylg.

Metagene 467; 39309_at Cluster Incl. M88714:Human bradykinin receptor (BK-2) mRNA, complete cd; 40684_at Cluster Incl. U78190:Human GTP cyclohydrolase I feedback regulatory pro; 41718_g_at Cluster Incl. AC004770:Homo sapiens chromosome 11, BAC CIT-HSP-311e8; 34738_at Cluster Incl. L11931:Human cytosolic serine hydroxymethyltransferase (S; 33931_at Cluster Incl. X71973:H. sapiens GPx-4 mRNA for phospholipid hydroperoxid; 36945_at Cluster Incl. X94910:Homo sapiens mRNA for ERp28 protein /cds=(11,796); 38426_at Cluster Incl. X83928:H. sapiens mRNA for transcription factor TFIID subu.

Metagene 468; 31633_g_at Cluster Incl. Z95118:Human DNA sequence from clone 354J5 on chromosom; 33024_at Cluster Incl. M69136:Human heart chymase mRNA, complete cds /cds=(15,75; 36431_at Cluster Incl. AL109709:Homo sapiens mRNA full length insert cDNA clone; 37870_at Cluster Incl. AF073771:Homo sapiens RNA polymerase II termination facto; 38570_at Cluster Incl. X03066:Human mRNA for HLA-D class II antigen DO beta chai; 32061_at Cluster Incl. U96629:Human chromosome 8 BAC clone CIT987SK-2A8 complete; 32651_at Cluster Incl. W28770:51d8 Homo sapiens cDNA /gb=W28770 /gi=1308718 /ug=; 35671_at Cluster Incl. U02619:Human TFIIIC Box B-binding subunit mRNA, complete; 1331_s_at U83598 /FEATURE= /DEFINITION=HSU83598 Human death domain receptor 3 so.

Metagene 469; 40448_at Cluster Incl. M92843:H. sapiens zinc finger transcriptional regulator mR; 32583_at Cluster Incl. J04111:Human c-jun proto oncogene (JUN), complete cds, cl; 2094_s_at K00650 /FEATURE=cds /DEFINITION=HUMFOS Human fos proto-oncogene (c-fos; 1915_s_at V01512 /FEATURE=mRNA#1 /DEFINITION=HSCFOS Human cellular oncogene c-fo; 1916_s_at V01512 /FEATURE=mRNA#2 /DEFINITION=HSCFOS Human cellular oncogene c-fo; 1005_at X68277 /FEATURE=cds /DEFINITION=HSCL100 H. sapiens CL 100 mRNA for protei.

Metagene 470; 31443_at Cluster Incl. S76346:AML1=AML1 {alternatively spliced, exons 5 and b} [; 31497_at Cluster Incl. U19142:Human GAGE-1 protein mRNA, complete cds /cds=(48,4; 37077_at Cluster Incl. D13243:Homo sapiens gene for pyruvate kinase L /cds=(39,1; 37498_at Cluster Incl. AL050404:Human DNA sequence from clone 955M13 on chromoso; 38181_at Cluster Incl. X57766:Human stromelysin-3 mRNA /cds=(9,1475) /gb=X57766; 35219_at Cluster Incl. AL050202:Homo sapiens mRNA; cDNA DKFZp586E2023 (from clon; 37270_at Cluster Incl. AF007876:Homo sapiens Na,K-ATPase beta 2 subunit gene, co; 39862_at Cluster Incl. AA528252:nh92c11.s1 Homo sapiens cDNA, 3 end /clone=IMAG; 40277_at Cluster Incl. AI799984:wc46f12.x1 Homo sapiens cDNA, 3 end /clone=IMAG; 1072_g_at M77810 /FEATURE= /DEFINITION=HUMGATA2A Human transcription factor GATA.

Metagene 471; 35467_g_at Cluster Incl. W73046:zd54h09.r1 Homo sapiens cDNA, 5 end /clone=IMAG; 32808_at Cluster Incl. X07979:Human mRNA for integrin beta 1 subunit /cds=(103,2.

Metagene 472; 32372_at Cluster Incl. L22569:Homo sapiens cathepsin B mRNA, 3 UTR with a stem-1; 38849_at Cluster Incl. AF038966:Homo sapiens secretory carrier-associated membra; 32125_at Cluster Incl. AA928996:oo27f06.s1 Homo sapiens cDNA, 3 end /clone=IMAG; 32144_at Cluster Incl. AL050135:Homo sapiens mRNA; cDNA DKFZp586K091 (from clone; 33297_at Cluster Incl. AL031778:dJ34B21.3 (PUTATIVE novel protein) /cds=(297,557; 38665_at Cluster Incl. D85939:Homo sapiens mRNA for p97 homologous protein, comp; 32777_at Cluster Incl. Y12478:H. sapiens mRNA for CHD5 protein /cds=(42,566) /gb=; 33451_s_at Cluster Incl. AI526079:DU3.2-7.G09 Homo sapiens cDNA, 3 end /clone-e; 35292_at Cluster Incl. Z37166:H. sapiens BAT1 mRNA for nuclear RNA helicase (DEAD; 36639_at Cluster Incl. AF067853:Homo sapiens adenylosuccinate lyase (ADSL) mRNA,; 37010_at Cluster Incl. AI203737:qf76b12.x1 Homo sapiens cDNA, 3 end /clone=IMAG; 37672_at Cluster Incl. Z72499:H. sapiens mRNA for herpesvirus associated ubiquiti; 38097_at Cluster Incl. AF010313:Homo sapiens Pig8 (PIG8) mRNA, complete cds /cds; 38368_at Cluster Incl. U31930:Human deoxyuridine nucleotidohydrolase mRNA, compl; 38413_at Cluster Incl. D15057:Human mRNA for DAD-1, complete cds /cds=(66,407) /; 38455_at Cluster Incl. AL049650:dJ734P14.2.1 (snRNP (small nuclear ribonucleopro; 39131_at Cluster Incl. N36842:yy35g03.s1 Homo sapiens cDNA, 3 end /clone=IMAGE-; 39924_at Cluster Incl. AB020660:Homo sapiens mRNA for KIAA0853 protein, partial; 40953_at Cluster Incl. S80562:acidic calponin [human, kidney, mRNA, 1607 nt]/cd; 41308_at Cluster Incl. U37408:Homo sapiens phosphoprotein CtBP mRNA, complete cd; 41328_s_at Cluster Incl. AL096717:Homo sapiens mRNA; cDNA DKFZp564P0662 (from cl; 32585_at Cluster Incl. AF027299:Homo sapiens protein 4.1-G mRNA, complete cds /c; 33117_r_at Cluster Incl. AA977163:oq25a04.s1 Homo sapiens cDNA, 3 end /clone=IM; 1151_at Epstein-Barr Virus Small Rna-Associated Protein; 989_at X17576 /FEATURE=cds /DEFINITION=HSNCK Human melanoma mRNA for nck protein; 735_-sat Protein Kinase Ht31, Camp-Dependent.

Metagene 473; 39230_at Cluster Incl. AL022318:bK150C2.2 (Phorbolin 3) /cds=(29,1177) /gb=AL022; 36503_at Cluster Incl. AB002409:Homo sapiens mRNA for SLC, complete cds /cds=(58.

Metagene 474; 36377_at Cluster Incl. U43672:Human putative transmembrane receptor IL-1Rrp mRNA; 39229_at Cluster Incl. W27883:39b10 Homo sapiens cDNA /gb=W27883 /gi=1307831 /ug; 32653_at Cluster Incl. AW020536:df11b12.y1 Homo sapiens cDNA, 5 end /clone=IMAG; 34677_f_at Cluster Incl. AJ012755:Homo sapiens mRNA for TL132 /cds=(1241,2305) /; 39863_at Cluster Incl. AB002294:Human mRNA for KIAA0296 gene, complete cds /cds=; 1927_s_at U77713 /FEATURE=mRNA /DEFINITION=HSALK07 Human activin receptor like k; 1351_at U07695 /FEATURE= /DEFINITION=HSU07695 Human tyrosine kinase (HTK) mRNA,; 288_s_at L25931 /FEATURE= /DEFINITION=HUMLBR Human lamin B receptor (LBR) mRNA,.

Metagene 475; 35384_at Cluster Incl. Z34897:H. sapiens mRNA for H1 histamine receptor /cds=(0,1; 35230_at Cluster Incl. AF070530:Homo sapiens clone 24751 unknown mRNA /cds=(0,12; 39353_at Cluster Incl. AI912041:wd84b06.x1 Homo sapiens cDNA, 3 end /clone=IMAG; 40788_at Cluster Incl. U84371:Human adenylate kinase 2A (AK2A) mRNA, complete cd; 35759_at Cluster Incl. AF026166:Homo sapiens chaperonin-containing TCP-1 beta su; 36941_at Cluster Incl. U16954:Human (AF1q) mRNA, complete cds /cds=(355,627) /gb; 38421_at Cluster Incl. AF070546:Homo sapiens clone 24607 mRNA sequence /cds=UNKN; 39199_at Cluster Incl. W28661:49h1 Homo sapiens cDNA /gb=W28661 /gi=1308609 /ug=; 40979_at Cluster Incl. AJ243310:Homo sapiens mRNA for C14orf3 protein /cds=(131,; 32529_at Cluster Incl. X69910:H. sapiens p63 mRNA for transmembrane protein /cds=; 1636_g_at U07563 /FEATURE=Poly_A_Site#1 /DEFINITION=HSABLGR3 Human proto-oncogen.

Metagene 476; 41658_at Cluster Incl. AF001436:Human clone zeta unknown protein mRNA, complete; 37194_at Cluster Incl. M68891:Human GATA-binding protein (GATA2) m-RNA, complete; 40468_at Cluster Incl. AB011126:Homo sapiens mRNA for KIAA0554 protein, partial; 41493_at Cluster Incl. AI094610:oy64f07.s1 Homo sapiens cDNA, 3 end /clone=IMAG; 41834_g_at Cluster Incl. AB016492:Homo sapiens hJTB gene, complete cds /cds=(464; 431_at X02530 /FEATURE=cds /DEFINITION=HSINFGER Human mRNA for gamma-interferon.

Metagene 477; 32010_at Cluster Incl. Z82180:Human DNA sequence from clone E81G9 on chromosome; 32423_at Cluster Incl. U48408:Human kidney water channel (hKID) mRNA, complete c; 33096_r_at Cluster Incl. U48224:Human beaded filament protein CP49 (LIFL-L) mRNA; 33950_g_at Cluster Incl. AF011406:Homo sapiens corticotropin releasing hormone r; 34047_at Cluster Incl. AF016045:Homo sapiens OVO-like 1 binding protein (OVOL1); 35445_at Cluster Incl. AC002398:Human DNA from chromosome 19-specific cosmid F25; 33712_at Cluster Incl. N63574:yy63f05.s1 Homo sapiens cDNA, 3 end /clone=IMAGE-; 35194_at Cluster Incl. X53463:Human mRNA for glutathione peroxidase-like protein; 36557_at Cluster Incl. M92303:Human voltage-dependent calcium channel beta-1 sub; 37926_at Cluster Incl. D14520:Human mRNA for GC-Box binding protein BTEB2, compl; 39915_at Cluster Incl. AB001535:Homo sapiens mRNA, complete cds /cds=(445,4956); 1223_at X66362 /FEATURE=cds /DEFINITION=HSSTHPKG H. sapiens mRNA PCTAIRE-3 for se.

Metagene 478; 35042_at Cluster Incl. L12398:Homo sapiens dopamine D4 receptor (DRD4) mRNA (D4.; 37166_at Cluster Incl. Z29481:H. sapiens mRNA for 3-hydroxyanthranilic acid dioxy; 40294_at Cluster Incl. U66676:HSU66676 Homo sapiens cDNA /gb=U66676 /gi=1906561; 41079_at Cluster Incl. AB010575:Homo sapiens mRNA for sodium channel, complete c; 36546_r_at Cluster Incl. AB011114:Homo sapiens mRNA for KIAA0542 protein, comple; 36815_at Cluster Incl. AF038185:Homo sapiens clone 23700 mRNA sequence /cds=UNKN; 37993_at Cluster Incl. X63422:H. sapiens mRNA for delta-subunit of mitochondrial; 40415_at Cluster Incl. X14813:Human liver mRNA for 3-oxoacyl-CoA thiolase /cds=(; 32757_at Cluster Incl. U84720:Homo sapiens mRNA export protein (RAE1) mRNA, comp; 32852_at Cluster Incl. U78678:Human thioredoxin mRNA, nuclear gene encoding mito; 38449_at Cluster Incl. W28931:56f3 Homo sapiens cDNA /gb=W28931 /gi=1309086 /ug=; 33215_g_at Cluster Incl. Y11681:Homo sapiens mRNA formitochondrial ribosomal pr; 870_f_at M93311 /FEATURE=cds /DEFINITION=HUMMETIII Human metallothionein-III gen.

Metagene 479; 35526_at Cluster Incl. K02766:Human complement component C9 mRNA, complete cds /; 33261_at Cluster Incl. M16941:Human MHC class II HLA-DR7-associated glycoprotein; 36623_at Cluster Incl. AB011406:Homo sapiens mRNA for alkalin phosphatase, compl; 38784_g_at Cluster Incl. J05581:Human polymorphic epithelial mucin (PEM) mRNA, c; 33197_at Cluster Incl. U39226:Human myosin VIIA (USH1B) mRNA, complete cds /cds=.

Metagene 480; 32884_at Cluster Incl. L09749:Homo sapiens (clone F4) transmembrane protein mRNA; 36491_at Cluster Incl. D82345:Homo sapiens mRNA for NB thymosin beta, complete c; 35341_at Cluster Incl. U90547:Human Ro/SSA ribonucleoprotein homolog (RoRet) mRN; 41822_at Cluster Incl. AF060503:Homo sapiens zinc finger protein (ZF5128) mRNA,; 1924_at U11791 /FEATURE= /DEFINITION=HSU11791 Human cyclin H mRNA, complete cds; 966_at X97795 /FEATURE=cds /DEFINITION=HSRAD54 H. sapiens mRNA homologous to S. c.

Metagene 481; 35038_at Cluster Incl. Y10129:H. sapiens mybpc3 gene /cds=(33,3857) /gb=Y10129 /g; 39674_r_at Cluster Incl. AB011792:Homo sapiens mRNA for extracellular matrix pro; 35141_at Cluster Incl. Z97029:Homo sapiens mRNA for ribonuclease H I large subun; 33345_at Cluster Incl. AF035621:Homo sapiens kinesin-related protein (KIF3C) mRN; 41269_r_at Cluster Incl. Y15906:Homo sapiens mRNA for XAGL protein /cds=(132,164.

Metagene 482; 34650_at Cluster Incl. U36798:Homo sapiens platelet cGI-PDE mRNA, complete cds /; 32974_at Cluster Incl. U07563:Human ABL gene, exon 1b and intron 1b, and putativ; 37818_at Cluster Incl. Y10936:H. sapiens mRNA for hypothetical protein downstream; 35732_at Cluster Incl. AL031427:dJ167A19.4 (novel protein) /cds=(0,1286) /gb=AL0; 36031_at Cluster Incl. AB024401:Homo sapiens mRNA for p33, complete cds /cds=(45; 36460_at Cluster Incl. AF008442:Homo sapiens RNA polymerase I subunit hRPA39 mRN; 39773_at Cluster Incl. W28235:43h8 Homo sapiens cDNA /gb=W28235 /gi=1308183 /ug=; 40077_at Cluster Incl. Z11559:H. sapiens mRNA for iron regulatory factor /cds=(10.

Metagene 483; 40333_at Cluster Incl. U43842:Homo sapiens bone morphogenetic protein-4 (hBMP-4); 836_at U43148 /FEATURE= /DEFINITION=HSU43148 Human patched homolog (PTC) mRNA, c.

Metagene 484; 39588_at Cluster Incl. AF055872:Homo sapiens Apo3 /DR3 ligand (APO3L) mRNA, compl; 37189_at Cluster Incl. AL023553:dJ347H13.3 (phosphomannomutase 1 (PMMH-22, yeast; 37596_at Cluster Incl. U09117:Human phospholipase c delta 1 mRNA, complete cds /; 38359_at Cluster Incl. Y12336:H. sapiens mRNA for F25B3.3 kinase like protein fro; 34859_at Cluster Incl. Z98046:dJ14O9.2 (Melanoma-Associated Antigen MAGE LIKE) /; 37016_at Cluster Incl. D13900:Homo sapiens mRNA for mitochondrial short-chain en; 38795_s_at Cluster Incl. X56687:H. sapiens mRNA for autoantigen NOR-90 /cds=(179,; 38845_at Cluster Incl. R89044:ym99b08.s1 Homo sapiens cDNA, 3 end /clone=IMAGE-; 1782_s_at M31303 /FEATURE=mRNA /DEFINITION=HUMOP18A Human oncoprotein 18 (Op18).

Metagene 485; 31636_s_at Cluster Incl. U09210:Human vesicular acetylcholine transporter mRNA,; 34557_at Cluster Incl. X67594:H. sapiens mRNA for MSH receptor /cds=(168,1121) /g; 35674_at Cluster Incl. AB023211:Homo sapiens mRNA for KIAA0994 protein, partial; 36643_at Cluster Incl. L20817:Homo sapiens tyrosine protein kinase (CAK) gene, c; 39828_at Cluster Incl. AA477714:zu44e09.s1 Homo sapiens cDNA, 3 end /clone=IMAG; 1681_at X03635 /FEATURE=cds /DEFINITION=HSERR Human mRNA for oestrogen receptor; 780_at X90978 /FEATURE= /DEFINITION=HSRNAML13 H. sapiens mRNA for an acute myeloi; 164_at U47931 /FEATURE=mRNA /DEFINITION=HSU47931 Human G-protein beta-3 subunit.

Metagene 486; 35497_at Cluster Incl. AI924594:wn57a11.x1 Homo sapiens cDNA, 3 end /clone=IMAG; 36371_at Cluster Incl. L35251:Homo sapiens extracellular matrix protein (MFAP3); 36442_g_at Cluster Incl. U02632:Human calcium-activated potassium channel mRNA,; 38503_at Cluster Incl. M63967:Human mitochondrial aldehyde dehydrogenase x gene,; 32626_at Cluster Incl. M90516:Human glutamine-fructose-6-phosphate amidotransfer; 36467_g_at Cluster Incl. U26742:Human dystrobrevin-delta mRNA, complete cds /cds; 40148_at Cluster Incl. U62325:Human FE65-like protein (hFE65L) mRNA, partial cds; 32229_at Cluster Incl. AF038957:Homo sapiens translation initiation factor 4e mR; 41499_at Cluster Incl. X15218:Human ski oncogene mRNA /cds=(72,2258) /gb=X15218; 1335_at X04434 /FEATURE=cds /DEFINITION=HSIGFRR Human mRNA for insulin-like gro; 785_at U96114 /FEATURE= /DEFINITION=HSU96114 Homo sapiens Nedd-4-like ubiquitin-; 369_s_at Z29331 /FEATURE=cds /DEFINITION=HSUCEH3 H. sapiens (23k/3) mRNA for ubiq.

Metagene 487; 37078_at Cluster Incl. J04132:Human T cell receptor zeta-chain mRNA, complete cd; 37196_at Cluster Incl. X79981:H. sapiens VE-cadherin mRNA /cds=(24,2378) /gb=X799; 37897_s_at Cluster Incl. AI985964:wr79d08.x1 Homo sapiens cDNA, 3 end /clone=IM; 41577_at Cluster Incl. AB020630:Homo sapiens mRNA for KIAA0823 protein, partial; 32580_at Cluster Incl. AF035152:Homo sapiens regulator of G-protein signalling 1.

Metagene 488; 32393_s_at Cluster Incl. W27466:31c9 Homo sapiens cDNA /gb=W27466 /gi=1307270 /u; 41417_at Cluster Incl. AC003108:Human Chromosome 16 BAC clone CIT987SK-327O24 /c; 41453_at Cluster Incl. U49089:Human neuroendocrine-dlg (NE-dlg) mRNA, complete c; 41635_at Cluster Incl. D14661:Human mRNA for KIAA0105 gene, complete cds /cds=(1; 41855_at Cluster Incl. AF030424:Homo sapiens histone acetyltransferase 1 mRNA, c; 31879_at Cluster Incl. U69127:Human FUSE binding protein 3 (FBP3) mRNA, partial; 32665_at Cluster Incl. AJ005801:Homo sapiens mRNA for protein phosphatase 2C (be; 33741_at Cluster Incl. AI741756:wg22e12.x1 Homo sapiens cDNA, 3 end /clone=IMAG; 33811_at Cluster Incl. AI761567:wg66a05.x1 Homo sapiens cDNA, 3 end /clone=IMAG; 34192_at Cluster Incl. AB011104:Homo sapiens mRNA for KIAA0532 protein, partial; 34258_at Cluster Incl. W28205:43g5 Homo sapiens cDNA /gb=W28205 /gi=1308171 /ug=; 34274_at Cluster Incl. AB029039:Homo sapiens mRNA for KIAA1116 protein, complete; 34723_at Cluster Incl. U79270:Human clone 23707 mRNA, partial cds /cds=(0,460) /; 35177_at Cluster Incl. AB018268:Homo sapiens mRNA for KIAA0725 protein, partial; 35702_at Cluster Incl. M76665:Human 11-beta-hydroxysteroid dehydrogenase (HSD11); 35973_at Cluster Incl. AB023163:Homo sapiens mRNA for KIAA0946 protein, partial; 38353_at Cluster Incl. AF042378:Homo sapiens spindle pole body protein spc98 hom; 39005_s_at Cluster Incl. AB018257:Homo sapiens mRNA for KIAA0714 protein, partia; 39794_at Cluster Incl. D29956:Human mRNA for KIAA0055 gene, complete cds /cds=(3; 40801_at Cluster Incl. AA643063:nr95e11.s1 Homo sapiens cDNA, 3 end /clone=IMAG; 41733_at Cluster Incl. AC003007:Human Chromosome 16 BAC clone CIT987SK-A-61E3 /c; 33368_at Cluster Incl. X76040:H. sapiens mRNA for Lon protease-like protein /cds=; 34359_at Cluster Incl. AA524058:ng33b12.s1 Homo sapiens cDNA, 3 end /clone=IMAG; 34401_at Cluster Incl. L32977:Homo sapiens (clone f17252) ubiquinol cytochrome c; 34813_at Cluster Incl. AL079283:Homo sapiens mRNA full length insert cDNA clone; 35356_at Cluster Incl. W21884:58c2 Homo sapiens cDNA /clone=(not-directional) /g; 35364_at Cluster Incl. U50939:Human amyloid precursor protein-binding protein 1; 37352_at Cluster Incl. M60618:Human nuclear autoantigen (SP-100) mRNA, complete; 37357_at Cluster Incl. D00723:Homo sapiens mRNA for hydrogen carrier protein, a; 37389_at Cluster Incl. AI346580:qp51f08.x1 Homo sapiens cDNA, 3 end /clone=IMAG; 37715_at Cluster Incl. AF045184:Homo sapiens nuclear receptor coactivator NCoA-6; 37752_at Cluster Incl. M15353:Homo sapiens cap-binding protein mRNA, complete cd; 37755_at Cluster Incl. AB023169:Homo sapiens mRNA for KIAA0952 protein, complete; 37762_at Cluster Incl. Y07909:H. sapiens mRNA for Progression Associated Protein; 40229_at Cluster Incl. AJ010071:Homo sapiens for TOM1-like protein /cds=(30,1460; 40577_at Cluster Incl. AI951046:wx62g06.x1 Homo sapiens cDNA, 3 end /clone=IMAG; 41557_at Cluster Incl. D29641:Human mRNA for KIAA0052 gene, partial cds /cds=(0,; 32541_at Cluster Incl. S46622:calcineurin A catalytic subunit [human, testis, mR; 1891_at D14497 /FEATURE= /DEFINITION=HUMPOPSTK Human mRNA for proto-oncogene pro; 674_g_at J04031 /FEATURE= /DEFINITION=HUMMDMCSF Human methylenetetrahydrofolate.

Metagene 489; 31554_at Cluster Incl. X55019:H. sapiens mRNA for acetylcholine receptor delta su; 31930_f_at Cluster Incl. X63096:H. sapiens mRNA for rhesus polypeptide (RhVIII) /; 40381_at Cluster Incl. AB023189:Homo sapiens mRNA for KIAA0972 protein, complete; 31821_at Cluster Incl. AB011167:Homo sapiens mRNA for KIAA0595 protein, partial; 38724_at Cluster Incl. AB011087:Homo sapiens mRNA for KIAA0515 protein, partial; 39735_at Cluster Incl. AF069987:Homo sapiens nitrilase 1 (NIT1) mRNA, complete c; 33934_at Cluster Incl. AB018340:Homo sapiens mRNA for KIAA0797 protein, partial; 37734_at Cluster Incl. D80006:Human mRNA for KIAA0184 gene, partial cds /cds=(0,; 40538_at Cluster Incl. AL050358:Homo sapiens mRNA; cDNA DKFZp564M1916 (from clon; 1071_at M77810 /FEATURE= /DEFINITION=HUMGATA2A Human transcription factor GATA-2; 948_s_at D63861 /FEATURE=expanded_cds /DEFINITION=D63861 Homo sapiens DNA for cy.

Metagene 490; 32004_s_at Cluster Incl. W32483:zc67e07.r1 Homo sapiens cDNA, 5 end /clone=IMAG; 33679_f_at Cluster Incl. X02344:Homo sapiens beta 2 gene /cds=(0,1337) /gb=X0234; 41717_at Cluster Incl. AC004770:Homo sapiens chromosome 11, BAC CIT-HSP-311e8 (B; 33781_s_at Cluster Incl. AF075599:Homo sapiens ubiquitin conjugating enzyme 12 (; 34669_at Cluster Incl. X96717:H. sapiens mRNA for transcription factor TFE3 /cds=; 39685_at Cluster Incl. AL050282:Homo sapiens mRNA; cDNA DKFZp586H2219 (from clon; 40845_at Cluster Incl. U10324:Human nuclear factor NF90 mRNA, complete cds /cds=; 33414_at Cluster Incl. X57398:Human mRNA for pM5 protein /cds=(0,3572) /gb=X5739; 33818_at Cluster Incl. AC004472:Homo sapiens chromosome 9, P1 clone 11659 /cds=(; 36158_at Cluster Incl. AF086947:untitled /cds=(334,4119) /gb=AF086947 /gi=413912; 39159_at Cluster Incl. X99656:H. sapiens mRNA for protein containing SH3 domain,; 39518_at Cluster Incl. H97470:yw11b04.s1 Homo sapiens cDNA, 3 end /clone=IMAGE-; 39521_at Cluster Incl. U55054:Human K—Cl cotransporter (hKCC1) mRNA, complete cd; 40619_at Cluster Incl. M91670:Human ubiquitin carrier protein (E2-EPF) mRNA, com; 1813_at Epidermal Growth Factor Receptor-Related Protein; 1751_g_at AD000092 /FEATURE=cds#5 /DEFINITION=CH19HHR23 Homo sapiens DNA from ch; 1218_at X12794 /FEATURE=cds /DEFINITION=HSEAR2 Human v-erbA related ear-2 gene; 1224_at X66363 /FEATURE=cds /DEFINITION=HSSTHPKD H. sapiens mRNA PCTAIRE-1 for se; 922_at J02902 /FEATURE=mRNA /DEFINITION=HUMP2A Human protein phosphatase 2A regu; 893_at M91670 /FEATURE= /DEFINITION=HUME2EPI Human ubiquitin carrier protein (E2; 518_at U07132 /FEATURE= /DEFINITION=HSU07132 Human steroid hormone receptor Ner-; 423_at X66899 /FEATURE=cds /DEFINITION=HSEWS H. sapiens EWS mRNA; 391_at X89416 /FEATURE=cds /DEFINITION=HSRNAPPP5 H. sapiens mRNA for protein phos; 399_at X99325 /FEATURE=cds /DEFINITION=HSSTE20 H. sapiens mRNA for Ste20-like kin; 294_s_at Protein Kinase Pitslre, Alpha, Alt. Splice 1-Feb; 163_at U46461 /FEATURE= /DEFINITION=HSU46461 Human dishevelled homolog (DVL) mRN; 146_at U81802 /FEATURE= /DEFINITION=HSU81802 Human PtdIns 4-kinase (PI4Kb) mRNA,; 110_at X96753 /FEATURE=cds /DEFINITION=HSMCSP H. sapiens mRNA for melanoma-associ; 160044_g_at NM_{—}001098 /FEATURE=mRNA /DEFINITION=Homo sapiens aconitase 2, mitoch.

Metagene 491; 37418_at Cluster Incl. M36653:Human Oct-2 factor mRNA, complete cds /cds=UNKNOWN; 41152_f_at Cluster Incl. T89651:yd99a05.s1 Homo sapiens cDNA, 3 end /clone=IMAG; 36104_at Cluster Incl. AA526497:ni96d07.s1 Homo sapiens cDNA, 3 end /clone=IMAG; 1313_at D38048 /FEATURE= /DEFINITION=D38048 Human mRNA for proteasome subunit z,; 360_at Y11999 /FEATURE=cds /DEFINITION=HSINSP3KN H. sapiens mRNA for inositol 1,4.

Metagene 492; 35451_s_at Cluster Incl. AA280726:zs96g08.s1 Homo sapiens cDNA, 3 end /clone=IM; 35942_at Cluster Incl. AI183417:qd24c11.x1 Homo sapiens cDNA, 3 end /clone=IMAG; 36330_at Cluster Incl. Y17448:Homo sapiens CCBL1 gene, last two exons /cds=(0,14; 35253_at Cluster Incl. AB011143:Homo sapiens mRNA for KIAA0571 protein, complete; 38830_at Cluster Incl. U66685:HSU66685 Homo sapiens cDNA /gb=U66685 /gi=1906570; 1233_s_at M76125 /FEATURE= /DEFINITION=HUMTYRKINR Human tyrosine kinase receptor; 392_g_at X89416 /FEATURE=cds /DEFINITION=HSRNAPPP5 H. sapiens mRNA for protein ph; 144_at U80628 /FEATURE= /DEFINITION=HSU80628 Human thymidine kinase 2 isoform B.

Metagene 493; 41382_at Cluster Incl. AJ000342:Homo sapiens mRNA for DMBT1 6 kb transcript vari; 39144_at Cluster Incl. U59736:Human transcription factor (NFATc.b) mRNA, complet.

Metagene 494; 36368_at Cluster Incl. AF054998:Homo sapiens clone 24479 mRNA sequence /cds=UNKN; 34031_i_at Cluster Incl. U90268:Human Krit1 mRNA, complete cds /cds=(25,1614) /g; 36720_at Cluster Incl. AA873266:oh68e03.s1 Homo sapiens cDNA, 3 end /clone=IMAG; 38224_at Cluster Incl. U71300:Human snRNA activating protein complex 50 kD subuni; 33805_at Cluster Incl. AB007949:Homo sapiens mRNA for KIAA0480 protein, complete; 34718_at Cluster Incl. X04434:Human mRNA for insulin-like growth factor I recept; 39401_at Cluster Incl. W28264:44c9 Homo sapiens cDNA /gb=W28264 /gi=1308212 /ug=; 34381_at Cluster Incl. AI708889:as86g01.x1 Homo sapiens cDNA, 3 end /clone=IMAG; 35373_at Cluster Incl. M61906:Human P13-kinase associated p85 mRNA sequence /cds; 36573_at Cluster Incl. U78524:Human Gu binding protein mRNA, partial cds /cds=(0; 38765_at Cluster Incl. AB028449:Homo sapiens mRNA for Helicase-MOI, complete cds; 33173_g_at Cluster Incl. T75292:yc89b05.r1 Homo sapiens cDNA, 5 end /clone=IMAG; 1785_at S66431 /FEATURE= /DEFINITION=S66431 RBP2=retinoblastoma binding protein.

Metagene 495; 39628_at Cluster Incl. AI671547:wb33e07.x1 Homo sapiens cDNA, 3 end /clone=IMAG; 33704_at Cluster Incl. AB011111:Homo sapiens mRNA for KIAA0539 protein, complete; 36494_at Cluster Incl. AF058918:Homo sapiens unknown mRNA /cds=(212,634) /gb=AF0; 32572_at Cluster Incl. X98296:H. sapiens mRNA for ubiquitin hydrolase /cds=(59,77.

Metagene 496; 39316_at Cluster Incl. AI935153:wp14c12.x1 Homo sapiens cDNA, 3 end /clone=IMAG; 39647_s_at Cluster Incl. U95019:Human voltage-dependent calcium channel beta-2c; 1452_at U24576 /FEATURE= /DEFINITION=U24576 Homo sapiens breast tumor autoantige; 903_at L42373 /FEATURE=mRNA /DEFINITION=HUMPP2A Homo sapiens phosphatase 2A B56-.

Metagene 497; 41395_at Cluster Incl. AB003791:Homo sapiens mRNA for keratan sulfate Gal-6-sulf; 33272_at Cluster Incl. AA829286:of08a01.s1 Homo sapiens cDNA, 3 end /clone=IMAG; 36555_at Cluster Incl. AF044311:Homo sapiens gamma-synuclein gene, complete cds; 37587_at Cluster Incl. S43855:recoverin=photoreceptor protein [human, retina, mR; 38261_at Cluster Incl. AF085692:Homo sapiens multidrug resistance-associated pro; 41123_s_at Cluster Incl. L35594:Human autotaxin mRNA, complete cds /cds=(49,2796; 41124_r_at Cluster Incl. L35594:Human autotaxin mRNA, complete cds /cds=(49,2796; 34311_at Cluster Incl. X76648:H. sapiens mRNA for glutaredoxin /cds=(63,383) /gb=; 38118_at Cluster Incl. U73377:Human p66shc (SHC) mRNA, complete cds /cds=(194,19; 40541_at Cluster Incl. X01630:Human mRNA for argininosuccinate synthetase /cds=(; 1930_at U83659 /FEATURE= /DEFINITION=HSU83659 Human multidrug resistance-associa; 1731_at M21574 /FEATURE=mRNA /DEFINITION=HUMPDGFRAA Human platelet-derived growt; 707_s_at Mucin 6, Gastric; 411_i_at X57351 /FEATURE=cds /DEFINITION=HS18D Human 1-8D gene from interferon-i; 291_s_at J04152 /FEATURE=mRNA /DEFINITION=HUMGA733A Human gastrointestinal tumor.

Metagene 498; 32168_s_at Cluster Incl. U85267:Homo sapiens down syndrome candidate region 1 (D; 36638_at Cluster Incl. X78947:H. sapiens mRNA for connective tissue growth factor; 37028_at Cluster Incl. U83981:Homo sapiens apoptosis associated protein (GADD34); 38772_at Cluster Incl. Y11307:H. sapiens CYR61 mRNA /cds=(223,1368) /gb=Y11307 /g.

Metagene 499; 32424_at Cluster Incl. D84424:Homo sapiens mRNA for hyaluronan synthase, complet; 36214_at Cluster Incl. U70663:Human zinc finger transcription factor hEZF (EZF); 40375_at Cluster Incl. X63741:H. sapiens pilot mRNA /cds=(353,1516) /gb=X63741 /g; 32065_at Cluster Incl. S68134:CREM=cyclic AMP-responsive element modulator beta; 37623_at Cluster Incl. X75918:H. sapiens mRNA for NOT /cds=(317,2113) /gb=X75918; 33439_at Cluster Incl. D15050:Human mRNA for transcription factor AREB6, complet; 36669_at Cluster Incl. L49169:Human G0S3 mRNA, complete cds /cds=(593,1609) /gb=; 38037_at Cluster Incl. M60278:Human heparin-binding EGF-like growth factor mRNA,; 41483_s_at Cluster Incl. X56681:Human junD mRNA /cds=(174,1217) /gb=X56681 /gi=3; 547_s_at S77154 /FEATURE= /DEFINITION=S77154 TINUR=NGFI-B/nur77 beta-type trans; 279_at L13740 /FEATURE= /DEFINITION=HUMTR3A Human TR3 orphan receptor mRNA, comp; 280_g_at L13740 /FEATURE= /DEFINITION=HUMTR3A Human TR3 orphan receptor mRNA, co.

Metagene 500; 38913_at Cluster Incl. U60319:Homo sapiens haemochromatosis protein (HLA-H) mRNA; 39633_at Cluster Incl. Z18948:H. sapiens mRNA for S100E calcium binding protein /; 31868_at Cluster Incl. AF060798:Homo sapiens myristilated and palmitylated serin; 32632_g_at Cluster Incl. J03060:Human glucocerebrosidase (GCB) gene /cds=(229,17; 32670_at Cluster Incl. L38969:Homo sapiens thrombospondin 3 (THBS3) gene, comple; 34181_at Cluster Incl. X55330:H. sapiens mRNA for aspartylglucosaminidase /cds=(1; 36497_at Cluster Incl. W28438:47g10 Homo sapiens cDNA /gb=W28438 /gi=1308449 /ug; 38697_at Cluster Incl. AL050274:Homo sapiens mRNA; cDNA DKFZp566C243 (from clone; 39069_at Cluster Incl. AF053944:Homo sapiens aortic carboxypeptidase-like protei; 40136_at Cluster Incl. AB014576:Homo sapiens mRNA for KIAA0676 protein, partial; 32181_at Cluster Incl. M60922:Human surface antigen mRNA, complete cds /cds=(126; 36126_at Cluster Incl. U18919:Human chromosome 17q12-21 mRNA, clone pOV-2, parti; 39910_at Cluster Incl. AA663800:ae72g12.s1 Homo sapiens cDNA, 3 end /clone=IMAG; 32502_at Cluster Incl. AL041124:DKFZp434D0316_s1 Homo sapiens cDNA, 3 end /clon; 33132_at Cluster Incl. U37012:Human cleavage and polyadenylation specificity fac; 2020_at M73554 /FEATURE= /DEFINITION=HUMBCL1 Human bcl-1 mRNA, complete CDS.

Metagene 501; 34771_at Cluster Incl. AF035959:Homo sapiens type-2 phosphatidic acid phosphatas; 37974_at Cluster Incl. AL050178:Homo sapiens mRNA; cDNA DKFZp586J1822 (from clon; 39787_at Cluster Incl. AB029821:Homo sapiens mRNA for phosphatidylethanolamine N; 34866_at Cluster Incl. AF055029:Homo sapiens clone 24711 mRNA sequence /cds=UNKN; 33202_f_at Cluster Incl. U43747:Human frataxin (FRDA) mRNA, complete cds /cds=(5.

Metagene 502; 35503_at Cluster Incl. M81590:Homo sapiens serotonin 1D receptor (5-HT1D˜) mRNA,; 32332_at Cluster Incl. X69433:H. sapiens mRNA for mitochondrial isocitrate dehydr; 33480_at Cluster Incl. X15393:H. sapiens motilin gene exon 2 (and joined CDS) /cd; 37510_at Cluster Incl. AF036715:Homo sapiens syntaxin 8 mRNA, complete cds /cds=; 37880_at Cluster Incl. X53414:Human mRNA for peroxisomal L-alanine-glyoxylate am; 38517_at Cluster Incl. M87503:Human IFN-responsive transcription factor subunit; 38531_at Cluster Incl. AA428150:zw57c05.s1 Homo sapiens cDNA, 3 end /clone=IMAG; 38865_at Cluster Incl. AJ01736:Homo sapiens mRNA for growth factor receptor bin; 41108_at Cluster Incl. Y14391:Homo sapiens mRNA for putative GTP-binding protein; 36058_at Cluster Incl. AL096741:Homo sapiens mRNA; cDNA DKFZp586O0223 (from clon; 37214_g_at Cluster Incl. X90392:H. sapiens mRNA for DNase X gene /cds=(794,1702); 38966_at Cluster Incl. AF038958:Homo sapiens synaptic glycoprotein SC2 spliced v; 40089_at Cluster Incl. AJ224442:Homo sapiens mRNA for putative methyltransferase; 40104_at Cluster Incl. D63780:Homo sapiens mRNA for YSK1, complete cds /cds=(114; 40866_at Cluster Incl. AJ001258:Homo sapiens mRNA for NIPSNAP1 protein /cds=(254; 41776_at Cluster Incl. U70660:Human copper transport protein HAH1 (HAH1) mRNA, c; 35796_at Cluster Incl. Y17169:Homo sapiens mRNA for A6 related protein /cds=(104; 35835_at Cluster Incl. AB019409:Homo sapiens mRNA, expressed in fibroblasts of p; 36574_at Cluster Incl. Z68907:H. sapiens mRNA for NAD (H)-specific isocitrate deh; 37311_at Cluster Incl. AF010400:untitled /cds=(50,1063) /gb=AF010400 /gi=2612878; 37706_at Cluster Incl. U28811:Human cysteine-rich fibroblast growth factor recep; 38787_at Cluster Incl. X63131:H. sapiens My1 (PML) mRNA /cds=(141,2042) /gb=X6313; 39867_at Cluster Incl. S75463:P43=mitochondrial elongation factor homolog [human; 1980_s_at X58965 /FEATURE= /DEFINITION=HSNM23H2G H. sapiens RNA for nm23-H2 gene; 1855_at X14445 /FEATURE=expanded cds /DEFINITION=HSINT2 Human int-2 proto-oncoge; 982_at X74795 /FEATURE=cds /DEFINITION=HSP1CDC46 H. sapiens P1-Cdc46 mRNA.

Metagene 503; 31935_s_at Cluster Incl. U75968:Human clone C3 CHL1 protein (CHLR1) mRNA, altern; 34167_s_at Cluster Incl. AA984230:am82g03.s1 Homo sapiens cDNA, 3 end /clone=IM; 35124_at Cluster Incl. M62982:Human arachidonate 12-lipoxygenase mRNA, complete; 32879_at Cluster Incl. AL080233:Homo sapiens mRNA; cDNA DKFZp586L111 (from clone; 35013_at Cluster Incl. AF013512:untitled /cds=(106,1551) /gb=AF013512 /gi=265381; 35883_at Cluster Incl. X66079:H. sapiens Spi-B mRNA /cds=(5,793) /gb=X66079 /gi=3; 38558_at Cluster Incl. M29273:Human myelin-associated glycoprotein (MAG) mRNA, c; 36826_at Cluster Incl. X64037:H. sapiens mRNA for RNA polymerase II associated pr; 36883_at Cluster Incl. X14640:Human mRNA for keratin 13 /cds=(42,1418) /gb=X1464; 39802_at Cluster Incl. X72308:Homo sapiens mRNA for monocyte chemotactic protein; 33377_at Cluster Incl. X03168:Human mRNA for S-protein /cds=(61,1497) /gb=X03168; 34405_at Cluster Incl. U47927:Human isopeptidase T (ISOT) mRNA, complete cds /cd; 37339_at Cluster Incl. U20657:Human ubiquitin protease (Unph) proto-oncogene mRN; 40558_at Cluster Incl. W28227:43h1 Homo sapiens cDNA /gb=W28227 /gi=1308175 /ug=; 40598_at Cluster Incl. W20138:zb40d12.r1 Homo sapiens cDNA, 5 end /clone=IMAGE-; 41793_at Cluster Incl. AI288757:qm11h01.x1 Homo sapiens cDNA, 3 end /clone=IMAG; 1804_at X07730 /FEATURE= /DEFINITION=HSPSA Human mRNA for prostate specific anti; 912_s_at M21056 /FEATURE=cds /DEFINITION=HUMPLA2A2 Human pancreatic phospholipas.

Metagene 504; 31810_g_at Cluster Incl. Z21488:H. sapiens contactin mRNA /cds=(121,3177) /gb=Z21; 33155_at Cluster Incl. M95740:Human alpha-L-iduronidase gene /cds=(0,1961) /gb=M.

Metagene 505; 35101_at Cluster Incl. X52997:Human mRNA for platelet glycoprotein IX /cds=(222,; 35131_at Cluster Incl. J05213:Homo sapiens sialoprotein precursor (IBSP) mRNA, c; 37176_at Cluster Incl. U96078:Homo sapiens hyaluronoglucosaminidase 1 (HYAL1) m-R; 37929_at Cluster Incl. AB017563:Homo sapiens IGSF4 gene /cds=(0,1328) /gb=AB0175.

Metagene 506; 35941_f_at Cluster Incl. U91329:Human kinesin-like motor protein KIF1C mRNA, com; 41407_at Cluster Incl. L03411:Human RD protein (RD) mRNA, complete cds /cds=(86,; 35169_at Cluster Incl. AI982638:wt53c06.x1 Homo sapiens cDNA, 3 end /clone=IMAG; 35701_at Cluster Incl. AI038821:ox96d03.x1 Homo sapiens cDNA, 3 end /clone=IMAG; 35723_at Cluster Incl. D16581:Human mRNA for 8-oxo-dGTPase, complete cds /cds=(2; 38725_s_at Cluster Incl. N36295:yx99b12.r1 Homo sapiens cDNA, 5 end /clone=IMAG; 39074_at Cluster Incl. U79241:Human clone 23759 mRNA, partial cds /cds=(0,1315); 39342_at Cluster Incl. X94754:H. sapiens mRNA for yeast methionyl-tRNA synthetase; 39363_at Cluster Incl. AF042384:Homo sapiens BC-2 protein mRNA, complete cds /cd; 40056_at Cluster Incl. D87989:Human mRNA for UDP-galactose transporter related i; 40837_at Cluster Incl. M99436:Human transducin-like enhancer protein (TLE2) mRNA; 41163_at Cluster Incl. AL109672:Homo sapiens mRNA full length insert cDNA clone; 41732_at Cluster Incl. AA310786:EST181572 Homo sapiens cDNA, 5 end /clone=ATCC-; 33824_at Cluster Incl. X74929:H. sapiens KRT8 mRNA for keratin 8 /cds=(59,1510) /; 33906_at Cluster Incl. AB001740:Homo sapiens mRNA for p27, complete cds /cds=(20; 35269_at Cluster Incl. AF093420:Homo sapiens Hsp70 binding protein HspBP1 mRNA,; 35823_at Cluster Incl. M63573:Human secreted cyclophilin-like protein (SCYLP) mR; 36135_at Cluster Incl. U86602:Human nucleolar protein p40 mRNA, complete cds /cd; 36613_at Cluster Incl. U09585:Homo sapiens putative interferon-related protein (; 38076_at Cluster Incl. X69907:H. sapiens gene for mitochondrial ATP synthase c su; 38808_at Cluster Incl. D64154:Human mRNA for Mr 110,000 antigen, complete cds /c; 40956_at Cluster Incl. X90857:H. sapiens mRNA for −14 gene, containing globin reg; 41259_at Cluster Incl. AI553745:tn28c11.x1 Homo sapiens cDNA, 3 end /clone=IMAG; 41332_at Cluster Incl. D38251:Homo sapiens mRNA for RPB5 (XAP4), complete cds /c; 32518_at Cluster Incl. AF019767:Homo sapiens zinc finger protein (ZPR1) mRNA, co; 2035_s_at M55914 /FEATURE= /DEFINITION=HUMCMYCQ Human c-myc binding protein (MBP; 1878_g_at M13194 /FEATURE=mRNA /DEFINITION=HUMERCC1 Human excision repair protei; 1322_at U47677 /FEATURE=mRNA /DEFINITION=HSE2F1S03 Human transcription factor E2; 1309_at D26598 /FEATURE= /DEFINITION=HUMPSH1 Human mRNA for proteasome subunit H; 1154_at J02645 /FEATURE=mRNA /DEFINITION=HUMEIF2A Human translational initiation; 503_at U37690 /FEATURE= /DEFINITION=HSU37690 Human RNA polymerase II subunit (hs; 241_g_at M64231 /FEATURE=mRNA /DEFINITION=HUMSPERSYN Human spermidine synthase g.

Metagene 507; 36336_s_at Cluster Incl. AC005390:Homo sapiens chromosome 19, cosmid R31180 /cds; 37101_at Cluster Incl. AL050008:Homo sapiens mRNA; cDNA DKFZp564A063 (from clone; 40297_at Cluster Incl. AC005053:Homo sapiens BAC clone RG041D11 from 7q21 /cds=(; 40695_at Cluster Incl. J05272:Human IMP dehydrogenase type 1 mRNA complete cds /; 41415_at Cluster Incl. L36720:Homo sapiens bystin mRNA, complete cds /cds=(64,98; 31856_at Cluster Incl. Z24680:H. sapiens garp gene mRNA, complete CDS /cds=(94,20; 34273_at Cluster Incl. AI267373:aq64c09.x1 Homo sapiens cDNA /clone=IMAGE-203569; 34293_at Cluster Incl. AF004426:Homo sapiens microtubule-based motor (HsKIFC3) m; 37893_at Cluster Incl. AI828880:wj37b02.x1 Homo sapiens cDNA, 3 end /clone=IMAG; 38617_at Cluster Incl. D45906:Homo sapiens mRNA for LIMK-2, complete cds /cds=(1; 38969_at Cluster Incl. AI828168:wk32h09.x1 Homo sapiens cDNA, 3 end /clone=IMAG; 41728_at Cluster Incl. D63486:Human mRNA for KIAA0152 gene, complete cds /cds=(1; 34830_at Cluster Incl. W25986:17e7 Homo sapiens cDNA /gb=W25986 /gi=1306253 /ug=; 36678_at Cluster Incl. D21261:Human mRNA for KIAA0120 gene, complete cds /cds=(7; 38393_at Cluster Incl. D87434:Human mRNA for KIAA0247 gene, complete cds /cds=(2; 38417_at Cluster Incl. M91029:Human AMP deaminase (AMPD2) mRNA /cds=(0,2282) /gb; 40169_at Cluster Incl. AF057140:Homo sapiens cargo selection protein TIP47 (TIP4; 40635_at Cluster Incl. AF089750:Homo sapiens flotillin-1 mRNA, complete cds /cds; 40964_at Cluster Incl. Z46376:H. sapiens HK2 mRNA for hexokinase II /cds=(1490,42; 40986_s_at Cluster Incl. AA058852:zf65a11.s1 Homo sapiens cDNA, 3 end /clone=IM; 41246_at Cluster Incl. AI743134:wg87f07.x1 Homo sapiens cDNA, 3 end /clone=IMAG; 32562_at Cluster Incl. X72012:H. sapiens end mRNA for endoglin /cds=(281,2158) /g; 33131_at Cluster Incl. X70683:H. sapiens mRNA for SOX-4 protein /cds=(350,1774) /; 33143_s_at Cluster Incl. U81800:Homo sapiens monocarboxylate transporter (MCT3); 33214_at Cluster Incl. Y11681:Homo sapiens mRNA for mitochondrial ribosomal prot; 1693_s_at D11139 /FEATURE=exons#1-4 /DEFINITION=HUMTIMP Human gene for tissue in; 1385_at M77349 /FEATURE= /DEFINITION=HUMTGFBIG Human transforming growth factor-; 1138_at L20859 /FEATURE= /DEFINITION=HUMGLVR1X Human leukemia virus receptor 1 (; 1020-sat U85611 /FEATURE= /DEFINITION=HSU85611 Human DNA-PK interaction protein; 892_at M90657 /FEATURE= /DEFINITION=HUML6A Human tumor antigen (L6) mRNA, comple; 793_at X54936 /FEATURE=cds /DEFINITION=HSPLGF H. sapiens mRNA for placenta growth; 69 I_g_at J02783 /FEATURE=m-RNA /DEFINITION=HUMTHBP Human thyroid hormone binding; 404_at X52425 /FEATURE=mRNA /DEFINITION=HSIL4R Human IL-4-R mRNA for the interle; 333_s_at Single-Stranded Dna-Binding Protein Mssp-1.

Metagene 508; 33632_g_at Cluster Incl. AF023612:Homo sapiens Dim1p homolog mRNA, complete cds; 36450_at Cluster Incl. D13897:Human DNA for peptide YY, complete cds /cds=(91,36; 39426_at Cluster Incl. AF017789:Homo sapiens putative transcription factor CA150; 40449_at Cluster Incl. L23320:Human replication factor C large subunit mRNA, com; 34796_at Cluster Incl. X63679:H. sapiens mRNA for TRAMP protein /cds=(121,1245) /; 37030_at Cluster Incl. AB020694:Homo sapiens mRNA for KIAA0887 protein, partial; 37377_i_at Cluster Incl. M13452:Human lamin A mRNA, 3end /cds=(0,1547) /gb=M1345; 38031_at Cluster Incl. D21853:Human mRNA for KIAA0111 gene, complete cds /cds=(2; 38815_at Cluster Incl. Y08999:H. sapiens mRNA for Sop2p-like protein /cds=(33,114.

Metagene 509; 31864_at Cluster Incl. X98263:H. sapiens mRNA for M-phase phosphoprotein, mp.

Metagene 509; 31864_at Cluster Incl. X98263:H. sapiens mRNA for M-phase phosphoprotein, mp;

4. Discussion

Applicants have described a novel and unbiased approach for developing detailed genomic phenotypes that are exquisitely predictive for clinical disease states in human aorta samples. Importantly, the analysis is driven by the ability to predict phenotypic characteristics rather than merely identifying genes whose expression differs by an arbitrary amount for a given clinical phenotype. Applicants identified patterns of gene expression involving multiple, interacting genes that define the association with the clinical phenotypes. Most importantly, our analytic approach is rigorous in assessing the validity of these patterns through cross-validation analyses. This establishes the robust conclusion that these patterns of gene expression have the capacity to accurately predict the clinical phenotype, and hence underpins the inferred validity of the connection between these genes and the phenotype. The method described could serve as a model for studying other complex cardiovascular diseases where target tissues are genomically phenotyped to identify high priority candidate genes. These genes are now the focus of SNP studies and the basis for studying clinical patient populations.

The statistical methodology described in this report has been successfully applied to identify gene expression patterns capable of discriminating breast cancer samples on the basis of estrogen receptor status and predicting both recurrence and extent of lymph node metastasis in primary breast cancer. The use of metagenes in our statistical approach places the emphasis on the differential co-expression of multiple genes acting in concert—consonant with the biological model of complex diseases. The decision tree models used in this study showed considerable predictive value using the metagene patterns identified. In the cross validation analysis, applicants were able to correctly classify unknown samples with 93.5% accuracy for extent of disease severity and 93.6% accuracy for aortic location. Even with the heterogeneous nature of human aorta tissues and of atherosclerotic lesions therein, the metagenes identified through this approach clearly exhibit reliable discriminatory patterns. One point in the disease burden analysis is that applicants used sections from two different locations within multiple aortas. Because applicants did not use a single location, the inherent heterogeneity within an individual aorta could have influenced our results. However, the genes that predicted aortic location in our second analysis were quite different from those in our first analysis suggesting that location was not a major factor in the burden analysis.

Our disease burden analysis identified genes associated with minimal vs. severe atherosclerosis as measured, primarily, by the extent of advanced disease plaques. The samples included in the analysis were classified by quantitative measures of atherosclerosis identifying genes that reflect extent of disease. Although our analysis did identify genes whose expression is limited in the tissue (i.e. endothelium-derived), it may be biased toward genes expressed in the more abundant media, especially the inner media which may be most interactive with the atheromas. Still, applicants intentionally used the full thickness of the vessel wall in this initial analysis as the disease process likely involves complex interactions across the tissue layers through autocrine and other mechanisms. The expression patterns identified therefore may represent the net gene expression effect contributed from the different tissue layers.

The second analysis identified genes associated with location within the aortas. The regional differences within an aorta could be related to disease susceptibility given results from the PDAY study that showed atherosclerotic development progresses from the distal to the proximal aorta. Cornhill J F et al, Monogr Atheroscler. 1990; 15:13-19. In our analysis of the proximal and distal sections of the human aortas, there was no significant difference in the age of the donor patients and the atherosclerotic burden of the proximal and distal sections used by Sudan IV and raised lesion scores. This would seem to indicate that the differential gene expression patterns applicants found were indicative of location rather than age or presence of disease. This may reflect inherent differences in the mechanical stress from hemodynamic factors experienced by the different locations or possibly differences in the clonal origin of the cells populating the proximal or distal areas. See Nakashima Y, Raines E W, Plump A S, Breslow J L, Ross R. Upregulation of VCAM-1 and ICAM-1 at atherosclerosis-prone sites on the endothelium in the ApoE-deficient mouse. Arterioscler Thromb Vasc Biol. 1998; 18:842-851; Passerini A G, Polacek D C, Shi C, Francesco N M, Manduchi E, Grant G R, Pritchard W F, Powell S, Chang G Y, Stoeckert C J, Jr., Davies P F. Coexisting proinflammatory and antioxidative endothelial transcription profiles in a disturbed flow region of the adult porcine aorta. Proc Natl Acad Sci USA. 2004; 101:2482-2487; Schwartz S M, deBlois D, O'Brien E R. The intima. Soil for atherosclerosis and restenosis. Circ Res. 1995; 77:445-465.

Our first analysis for extent of disease identified genes reproducibly associated with atherosclerosis in the literature such as apoE, osteopontin, and olr1. See Chen M, Masaki T, Sawamura T. LOX-1, the receptor for oxidized low-density lipoprotein identified from endothelial cells: implications in endothelial dysfunction and atherosclerosis. Pharmacol Ther. 2002; 95:89-100; de Knijff P, van den Maagdenberg A M, Frants R R, Havekes L M. Genetic heterogeneity of apolipoprotein E and its influence on plasma lipid and lipoprotein levels. Hum Mutat. 1994; 4:178-194; Ilveskoski E, Perola M, Lehtimaki T, Laippala P, Savolainen V, Pajarinen J, Penttila A, Lalu K H, Mannikko A, Liesto K K, Koivula T, Karhunen P J. Age-dependent association of apolipoprotein E genotype with coronary and aortic atherosclerosis in middle-aged men: an autopsy study. Circulation. 1999; 100:608-613; Kwon H M, Hong B K, Kang T S, Kwon K, Kim H K, Jang Y, Choi D, Park H Y, Kang S M, Cho S Y, Kim H S. Expression of osteopontin in calcified coronary atherosclerotic plaques. J Korean Med Sci. 2000; 15:485-493; Mahley R W. Apolipoprotein E: cholesterol transport protein with expanding role in cell biology. Science. 1988; 240:622-630; Bini A, Mann K G, Kudryk B J, Schoen F J. Noncollagenous bone matrix proteins, calcification, and thrombosis in carotid artery atherosclerosis. Arterioscler Thromb Vasc Biol. 1999; 19:1852-1861. As one would expect from a biological context, these genes were upregulated in the diseased sections of the aorta. Importantly for our study, applicants also identified genes such as capg, gm2, mmp9 and ccrl2 that encode proteins whose function is consistent with a role in atherogenesis but have not been previously linked to atherosclerosis. CapG is a key regulatory protein for actin and membrane phospholipids within migrating phagocytes. See Witke W, Li W, Kwiatkowski D J, Southwick F S. Comparisons of CapG and gelsolin-null macrophages: demonstration of a unique role for CapG in receptor-mediated ruffling, phagocytosis, and vesicle rocketing. J Cell Biol. 2001; 154:775-784. The gene gm2 has been well studied in the oncology field for its role in cell proliferation, adhesion and chemotaxis. See Garner B, Priestman D A, Stocker R, Harvey D J, Butters T D, Platt F M. Increased glycosphingolipid levels in serum and aortae of apolipoprotein E gene knockout mice. J Lipid Res. 2002; 43:205-214; Gouni-Berthold I, Seul C, Ko Y, Hescheler J, Sachinidis A. Gangliosides GM1 and GM2 induce vascular smooth muscle cell proliferation via extracellular signal-regulated kinase 1/2 pathway. Hypertension. 2001; 38:1030-1037; Sachinidis A, Kraus R, Seul C, Meyer zu Brickwedde M K, Schulte K, Ko Y, Hoppe J, Vetter H. Gangliosides GM1, GM2 and GM3 inhibit the platelet-derived growth factor-induced signaling transduction pathway in vascular smooth muscle cells by different mechanisms. Eur J Cell Biol. 1996; 71:79-88. In addition, it has been shown to be present in aorta of apoE knock-out mice. The proteins MMP1, MMP2, MMP9 and MMP13 have all been associated with atheroma progression presumably through vascular remodeling. See Jeng A Y, Chou M, Sawyer W K, Caplan S L, Von Linden-Reed J, Jeune M, Prescott M F. Enhanced expression of matrix metalloproteinase-3, -12, and -13 mRNAs in the aortas of apolipoprotein E-deficient mice with advanced atherosclerosis. Ann NY Acad Sci. 1999; 878:555-558; Prescott M F, Sawyer W K, Von Linden-Reed J, Jeune M, Chou M, Caplan S L, Jeng A Y. Effect of matrix metalloproteinase inhibition on progression of atherosclerosis and aneurysm in LDL receptor-deficient mice overexpressing MMP-3, MMP-12, and MMP-13 and on restenosis in rats after balloon injury. Ann NY Acad Sci 1999; 878:179-190; Lee R T, Schoen F J, Loree H M, Lark M W, Libby P. Circumferential stress and matrix metalloproteinase 1 in human coronary atherosclerosis. Implications for plaque rupture. Arterioscler Thromb Vasc Biol. 1996; 16:1070-1073. The gene product of ccrl2 serves as a receptor for monocyte chemotactic protein 1 (MCP1) and other chemokines, and may be important for processes such as vascular infiltration by monocytes and intimal hyperplasia. See Perez de Lema G, Maier H, Nieto E, Vielhauer V, Luckow B, Mampaso F, Schlondorff D. Chemokine expression precedes inflammatory cell infiltration and chemokine receptor and cytokine expression during the initiation of murine lupus nephritis. J Am Soc Nephrol. 2001; 12:1369-1382; Bush E, Maeda N, Kuziel W A, Dawson T C, Wilcox J N, DeLeon H, Taylor W R. CC chemokine receptor 2 is required for macrophage infiltration and vascular hypertrophy in angiotensin II-induced hypertension. Hypertension. 2000; 36:360-363. An ontologic analysis of the gene list confirmed much of what has already been reported regarding the processes involved in atherosclerosis. Although, the number of genes related to cell growth/motility, transcriptional regulation and signal transduction seem to indicate that even in the advanced stages, the disease is active and proliferating. This would suggest that molecular, targeted interventions could still be of some benefit to this population of patients.

The second analysis looked for genes related to disease susceptibility. As before, there were genes whose translated products are directly associated with atherosclerosis such as sod3 and procr. See Laszik Z G, Zhou X J, Ferrell G L, Silva F G, Esmon C T. Down-regulation of endothelial expression of endothelial cell protein C receptor and thrombomodulin in coronary atherosclerosis. Am J Pathol. 2001; 159:797-802; Hink H U, Santanam N, Dikalov S, McCann L, Nguyen A D, Parthasarathy S, Harrison D G, Fukai T. Peroxidase properties of extracellular superoxide dismutase: role of uric acid in modulating in vivo activity. Arterioscler Thromb Vasc Biol. 2002; 22:1402-1408.

In addition, the analysis identified genes whose function has been described in other human disorders, but are involved in inflammatory, growth signaling, and cell-cell communication pathways that would be important for early atherosclerosis initiation and progression. Key examples are a number of homeobox-containing genes, that have been found in vascular smooth muscle and endothelial cells and have been linked to cellular proliferation, migration and differentiation as well as vascular remodeling. See Gorski D H, Walsh K. The role of homeobox genes in vascular remodeling and angiogenesis. Circ Res. 2000; 87:865-872. Another gene, gata2, is a transcription factor that regulates endothelin-1 production in endothelial cells. Endothelin-1 is a vasoactive peptide that has been highly associated with atherosclerosis. See Fadel B M, Boutet S C, Quertermous T. Endothelial cell-specific regulation of the murine endothelin-1 gene. J Cardiovasc Pharmacol. 2000; 35:S7-11; Kawana M, Lee M E, Quertermous E E, Quertermous T. Cooperative interaction of GATA-2 and AP1 regulates transcription of the endothelin-1 gene. Mol Cell Biol. 1995; 15:4225-4231.

An intriguing observation is the great percentage of genes that are related to regulation of transcription and signal transduction without a great number of genes related to inflammation. One explanation could be that the genes identified by our analysis could be upstream effectors that modulate the inflammatory pathways. For example, the identification of the homeobox genes and gata2 is intriguing given the potential role in modulation of VCAM1 and ICAM1—two proteins linked to atherosclerotic pre-lesion and early lesion formation. Homeobox genes, particularly the C class, have been associated with the increased and decreased expression of ICAM1. See Cillo C. HOX genes in human cancers. Invasion Metastasis. 1994; 14:38-49; Cillo C, Cantile M, Mortarini R, Barba P, Parmiani G, Anichini A. Differential patterns of HOX gene expression are associated with specific integrin and ICAM profiles in clonal populations isolated from a single human melanoma metastasis. Int J Cancer. 1996; 66:692-697. The gene, gata2 has been shown to mediate VCAM induction in response to thrombin, estrogen and glucocorticoids. See Minami T, Aird W C. Thrombin stimulation of the vascular cell adhesion molecule-1 promoter in endothelial cells is mediated by tandem nuclear factor-kappa B and GATA motifs. J Biol Chem. 2001; 276:47632-47641; Minami T, Abid M R, Zhang J, King G, Kodama T, Aird W C. Thrombin stimulation of vascular adhesion molecule-1 in endothelial cells is mediated by protein kinase C (PKC)-delta-NF-kappa B and PKC-zeta-GATA signaling pathways. J Biol Chem. 2003; 278:6976-6984; Simoncini T, Maffei S, Basta G, Barsacchi G, Genazzani A R, Liao J K, De Caterina R. Estrogens and glucocorticoids inhibit endothelial vascular cell adhesion molecule-1 expression by different transcriptional mechanisms. Circ Res. 2000; 87:19-25.

The identification of genes that predict atherosclerotic phenotypes, whether they are genes already known to function in the disease process or novel genes not previously linked to the disease, represents an important initial step towards an improved understanding of the disease. Our method as described is not meant initially to serve as a diagnostic test, but rather as a means to prioritize genes and allow us to focus our research efforts for identification of SNPs for large-scale analyses of gene variants. As well, identification of important genes advances our understanding of the biological pathways relevant to atherosclerosis. Importantly, our analytical approach may identify not only the initial steps in biological pathways but the secondary and tertiary events as well. As such, the analysis provides a much richer dataset than merely identifying the immediate effectors of a process. Many of the genes applicants have identified are likely to be causative and may be relevant to future therapeutic interventions.

Furthermore, by finding the polymorphisms within these high priority genes, applicants may begin to identify combinations of SNPs, when taken in concert with clinical cardiovascular risk factors that may lead to the development of new diagnostic and prognostic tools for cardiovascular events. Clearly, a next step must be to develop a deeper understanding of the biological pathways implicated by these analyses and to begin the process of investigating the role of these genes in the development of vascular disease.

The following attached documents, cited throughout the specification, are incorporated by reference:
1. Gibbons G H, Dzau V J. Molecular therapies for vascular diseases. Science. 1996; 272:689-693.
2. Libby P, Aikawa M. Stabilization of atherosclerotic plaques: new mechanisms and clinical targets. Nat Med. 2002; 8:1257-1262.
3. Ross R. The pathogenesis of atherosclerosis: a perspective for the 1990s. Nature. 1993; 362:801-809.
4. Schwartz S M, Virmani R, Rosenfeld M E. The good smooth muscle cells in atherosclerosis. Curr Atheroscler Rep. 2000; 2:422-429.
5. Cornhill J F, Herderick E E, Stary H C. Topography of human aortic sudanophilic lesions. Monogr Atheroscler. 1990; 15:13-19.
6. Cornhill J F, Barrett W A, Herderick E E, Mahley R W, Fry D L. Topographic study of sudanophilic lesions in cholesterol-fed minipigs by image analysis. Arteriosclerosis. 1985; 5:415-426.
7. Huang E, West M, Nevins J. Gene expression profiling for prediction of clinical characteristics of breast cancer. Recent Progress in Hormone Research. 2003; 55:55-73.
8. West M, Blanchette C, Dressman H, Huang E, Ishida S, Spang R, Zuzan H, Olson J A, Jr., Marks J R, Nevins J R. Predicting the clinical status of human breast cancer by using gene expression profiles. Proc Natl Acad Sci USA. 2001; 98:11462-11467.
9. Huang E, Cheng, S. H., Dressman, H., Pittman, J., Tsou, M. H., Horng, C. F., Bild, A., Iversen, E, S., Liao, M., Chen, C. M., West, M., Nevins, J. R., and Huang, A. T. Gene expression predictors of breast cancer outcomes. Lancet. 2003; 361:1590-1596.
10. Huang E, Ishida S, Pittman J, Dressman H, Bild A, Kloos M, D'Amico M, Pestell R, West M, Nevins J. Gene expression phenotypic models that predict the activity of oncogenic pathways. Nature Genetics. 2003; 34:226-230.
11. Nakashima Y, Raines E W, Plump A S, Breslow J L, Ross R. Upregulation of VCAM-1 and ICAM-1 at atherosclerosis-prone sites on the endothelium in the ApoE-deficient mouse. Arterioscler Thromb Vasc Biol. 1998; 18:842-851.
12. Passerini A G, Polacek D C, Shi C, Francesco N M, Manduchi E, Grant G R, Pritchard W F, Powell S, Chang G Y, Stoeckert C J, Jr., Davies P F. Coexisting proinflammatory and antioxidative endothelial transcription profiles in a disturbed flow region of the adult porcine aorta. Proc Natl Acad Sci USA. 2004; 101:2482-2487.
13. Schwartz S M, deBlois D, O'Brien E R. The intima. Soil for atherosclerosis and restenosis. Circ Res. 1995; 77:445-465.
14. Chen M, Masaki T, Sawamura T. LOX-1, the receptor for oxidized low-density lipoprotein identified from endothelial cells: implications in endothelial dysfunction and atherosclerosis. Pharmacol Ther. 2002; 95:89-100.
15. de Knijff P, van den Maagdenberg A M, Frants R R, Havekes L M. Genetic heterogeneity of apolipoprotein E and its influence on plasma lipid and lipoprotein levels. Hum Mutat. 1994; 4:178-194.
16. Ilveskoski E, Perola M, Lehtimaki T, Laippala P, Savolainen V, Pajarinen J, Penttila A, Lalu K H, Mannikko A, Liesto K K, Koivula T, Karhunen P J. Age-dependent association of apolipoprotein E genotype with coronary and aortic atherosclerosis in middle-aged men: an autopsy study. Circulation. 1999; 100:608-613.
17. Kwon H M, Hong B K, Kang T S, Kwon K, Kim H K, Jang Y, Choi D, Park H Y, Kang S M, Cho S Y, Kim H S. Expression of osteopontin in calcified coronary atherosclerotic plaques. J Korean Med Sci. 2000; 15:485-493.
18. Mahley R W. Apolipoprotein E: cholesterol transport protein with expanding role in cell biology. Science. 1988; 240:622-630.
19. Bini A, Mann K G, Kudryk B J, Schoen F J. Noncollagenous bone matrix proteins, calcification, and thrombosis in carotid artery atherosclerosis. Arterioscler Thromb Vasc Biol. 1999; 19:1852-1861.
20. Witke W, Li W, Kwiatkowski D J, Southwick F S. Comparisons of CapG and gelsolin-null macrophages: demonstration of a unique role for CapG in receptor-mediated ruffling, phagocytosis, and vesicle rocketing. J Cell Biol. 2001; 154:775-784.
21. Garner B, Priestman D A, Stocker R, Harvey D J, Butters T D, Platt F M. Increased glycosphingolipid levels in serum and aortae of apolipoprotein E gene knockout mice. J Lipid Res. 2002; 43:205-214.
22. Gouni-Berthold I, Seul C, Ko Y, Hescheler J, Sachinidis A. Gangliosides GM1 and GM2 induce vascular smooth muscle cell proliferation via extracellular signal-regulated kinase 1/2 pathway. Hypertension. 2001; 38:1030-1037.
23. Sachinidis A, Kraus R, Seul C, Meyer zu Brickwedde M K, Schulte K, Ko Y, Hoppe J, Vetter H. Gangliosides GM1, GM2 and GM3 inhibit the platelet-derived growth factor-induced signaling transduction pathway in vascular smooth muscle cells by different mechanisms. Eur J Cell Biol. 1996; 71:79-88.
24. Jeng A Y, Chou M, Sawyer W K, Caplan S L, Von Linden-Reed J, Jeune M, Prescott M F. Enhanced expression of matrix metalloproteinase-3, -12, and -13 mRNAs in the aortas of apolipoprotein E-deficient mice with advanced atherosclerosis. Ann NY Acad Sci. 1999; 878:555-558.
25. Prescott M F, Sawyer W K, Von Linden-Reed J, Jeune M, Chou M, Caplan S L, Jeng A Y. Effect of matrix metalloproteinase inhibition on progression of atherosclerosis and aneurysm in LDL receptor-deficient mice overexpressing MMP-3, MMP-12, and MMP-13 and on restenosis in rats after balloon injury. Ann NY Acad Sci. 1999; 878:179-190.
26. Lee R T, Schoen F J, Loree H M, Lark M W, Libby P. Circumferential stress and matrix metalloproteinase 1 in human coronary atherosclerosis. Implications for plaque rupture. Arterioscler Thromb Vasc Biol. 1996; 16:1070-1073.
27. Perez de Lema G, Maier H, Nieto E, Vielhauer V, Luckow B, Mampaso F, Schlondorff D. Chemokine expression precedes inflammatory cell infiltration and chemokine receptor and cytokine expression during the initiation of murine lupus nephritis. J Am Soc Nephrol. 2001; 12:1369-1382.
28. Bush E, Maeda N, Kuziel W A, Dawson T C, Wilcox J N, DeLeon H, Taylor W R. CC chemokine receptor 2 is required for macrophage infiltration and vascular hypertrophy in angiotensin II-induced hypertension. Hypertension. 2000; 36:360-363.
29. Laszik Z G, Zhou X J, Ferrell G L, Silva F G, Esmon C T. Down-regulation of endothelial expression of endothelial cell protein C receptor and thrombomodulin in coronary atherosclerosis. Am J Pathol. 2001; 159:797-802.
30. Hink H U, Santanam N, Dikalov S, McCann L, Nguyen A D, Parthasarathy S, Harrison D G, Fukai T. Peroxidase properties of extracellular superoxide dismutase: role of uric acid in modulating in vivo activity. Arterioscler Thromb Vasc Biol. 2002; 22:1402-1408.
31. Gorski D H, Walsh K. The role of homeobox genes in vascular remodeling and angiogenesis. Circ Res. 2000; 87:865-872.
32. Fadel B M, Boutet S C, Quertermous T. Endothelial cell-specific regulation of the murine endothelin-1 gene. J Cardiovasc Pharmacol. 2000; 35:S7-11.
33. Kawana M, Lee M E, Quertermous E E, Quertermous T. Cooperative interaction of GATA-2 and AP1 regulates transcription of the endothelin-1 gene. Mol Cell Biol. 1995; 15:4225-4231.
34. Cillo C. HOX genes in human cancers. Invasion Metastasis. 1994; 14:38-49.
35. Cillo C, Cantile M, Mortarini R, Barba P, Parmiani G, Anichini A. Differential patterns of HOX gene expression are associated with specific integrin and ICAM profiles in clonal populations isolated from a single human melanoma metastasis. Int J Cancer. 1996; 66:692-697.
36. Minami T, Aird W C. Thrombin stimulation of the vascular cell adhesion molecule-1 promoter in endothelial cells is mediated by tandem nuclear factor-kappa B and GATA motifs. J Biol Chem. 2001; 276:47632-47641.
37. Minami T, Abid M R, Zhang J, King G, Kodama T, Aird W C. Thrombin stimulation of vascular adhesion molecule-1 in endothelial cells is mediated by protein kinase C (PKC)-delta-NF-kappa B and PKC-zeta-GATA signaling pathways. J Biol Chem. 2003; 278:6976-6984.
38. Simoncini T, Maffei S, Basta G, Barsacchi G, Genazzani A R, Liao J K, De Caterina R. Estrogens and glucocorticoids inhibit endothelial vascular cell adhesion molecule-1 expression by different transcriptional mechanisms. Circ Res. 2000; 87:19-25.
39. Pittman J, Huang E, Nevins J, Wang Q, West M. Bayesian analysis of binary prediction tree models for retrospectively sampled outcomes. Biostatistics 2004; 1(1): 1-18.

The following reference is specifically incorporated by reference: Seo D, Wang T, Dressman H, Herderick E E, Iversen E S, Dong C, Vata K, Milano C A, Rigat F, Pittman J, Nevins J R, West M, Goldschmidt-Clermont P J. “Gene expression phenotypes of atherosclerosis.” Arterioscler Thromb Vasc Biol. 2004 October;24(10):1922-7. Instant FIGS. 11-13 correspond to FIGS. 2-4 of Seo et al., respectively, also incoporated by reference.

What is claimed is:

1. A method of estimating whether a sample is from tissue having an atherosclerotic phenotype, said method comprising:
(a) obtaining an expression profile for said sample from at least two of said genes listed in Table I;
(b) providing one or more predictive statistical tree models, each model including one or more nodes, each node representing a metagene, each node including a statistical predictive probability of the having an atherosclerotic phenotype, each metagene representing a dominant factor from a group of genes associated with having an atherosclerotic phenotype, wherein at least two genes in the group of genes are selected from those listed in Table I; and
(c) determining an estimate of the sample having the atherosclerotic phenotype by averaging the predictions of one or more of the tree models applied to the expression profile of the sample.

2. The method of claim 1, wherein at least two of the genes are selected from genes having Genbank accession numbers selected from Y09445, AF053233, U43185, AL050008, AB022718, L10333, M80634, AF044896, X78565, AB011143, X69819, J02947, U78095, D67029, AF013249, AB014574, L13939, L06797, D89077, Y08374, X02317, AB002365, AF084481, D34625, AB011103, AF041259, J05037, AF056087, U81800, AL050262, AB018271, J03011, D12485, U88629, U75308, J03600, AF004709, AB002361, X90858, Z29067, U00952, M80254, AF030339, AJ007395, AF013570, Z22555, L22524, Y07512, Y00093, AB007889, Y08136, L10678, Z98046, D79994, D87074, X81109, AL049946, U78556, M63603, X12451, U89606, AB029018, AF095791, X74039, X90976, U00802, X96752, Z49107, AL080235, AF051851, AF062075, AB000220, AB015718, X78817, AJ000534, M63835, M16336, U32324, M22324, X54162, U57911, M64571, AC005546, AC005546, Y13622, L76191, U60060, AJ011497, D64142, D26350, X15414, D87434, X79204, AB014513, U63127, S59184, X53587, Z84718, AF030409, J04621, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, X97074, D89016, AF022797, M33552, U09578, M21186, M64925, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663, M63193, D84110, AJ006973, AB002318, U51333, U09577 and U00672.

3. The method of claim 1, wherein at least two of the genes are selected from genes having Genbank accession numbers selected from Y09445, AF053233, U43185, AL050008, AB022718, L10333, M80634, AF044896, X78565, AB011143, X69819, J02947, U78095, D67029, AF013249, AB014574, L13939, L06797, D89077, Y08374, X02317, AB002365, AF084481, D34625, AB011103, AF041259, J05037, AF056087, U81800, AL050262, AB018271, J03011, D12485, U88629, U75308, J03600, AF004709, AB002361, X90858, Z29067, U00952, M80254, AF030339, AJ007395, AF013570, Z22555, L22524, Y07512, Y00093, AB007889, Y08136, L10678, Z98046, D79994, D87074, X81109, AL049946, U78556, M63603, X12451, U89606, AB029018, AF095791, X74039, X90976, U00802, X96752, Z49107, AL080235, AF051851, AF062075, AB000220, AB015718, X78817, AJ000534, M63835, M16336, U32324, M22324, X54162, U57911, M64571, AC005546, AC005546, Y13622, L76191, U60060, AJ011497, D64142, D26350, X15414, D87434, X79204, AB014513, U63127, S59184, X53587, Z84718, AF030409, J04621, U56833, J05070, AF093118, U12707, M55531, AB019527, X62055, D83004, X76534, U45285, X63657, L09708, AB020316, AF112219, Y14768, Y14768, Y14768, Y14768, Y14768, Y14768, AL031846, AL031846, AF036927, D49400, M55210, X97074, D89016, AF022797, M33552, U09578, M21186, M64925, U10906, U83993, AF022789, L35249, M61916, AB011155, X91809, U20158, S59049, U13991, X93498, M87770, AL050139, M73720, U35451, M32315, Y13710, AB008109, M60830, X71874, AB007972, X16663, M63193, D84110, AJ006973, AB002318, U51333, U09577 and U00672.

4. The method according to claim 1, wherein said tissue is a vascular tissue.

5. The method according to claim 2, wherein said vascular tissue is aortic tissue.

6. The method of claim 1, wherein the sample is from a mammal suspected of having tissue having an atherosclerotic phenotype.

7. The method of claim 6, wherein the mammal is at risk of being afflicted with atherosclerosis.

8. The method of claim 7, wherein the mammal has being treated with an anti-atherosclerosis agent.

9. The method of claim 1, wherein at least one metagene is Metagene n, wherein n is an integer between 1 and 509.

10. The method of claim 1, wherein the one or more predictive statistical tree models correctly classify samples with greater than 85% accuracy.

11. The method of claim 1, wherein the one or more predictive statistical tree models correctly classify samples with greater than 90% accuracy.

12. A method of predicting the susceptibility of a mammal for developing atherosclerosis, the method comprising:
(a) obtaining an expression profile of at least two of said genes listed in Table II from a sample from the mammal;
(b) providing one or more predictive statistical tree models, each model including one or more nodes, each node representing a metagene, each node including a statistical predictive probability of being susceptible to developing atherosclerosis, each metagene representing a dominant factor from a group of genes associated with susceptible to developing atherosclerosis, wherein at least two genes in the group of genes are selected from those listed in Table II; and
(c) determining an estimate of the sample being susceptible to developing atherosclerosis by averaging the predictions of one or more of the tree models applied to the expression profile of the sample.

13. The method of claim 12, wherein at least two of the genes are selected from genes having Genbank accession numbers selected from M68891, X51757, D83004, X06256, Z22865, X75918 and M55153.

14. The method of claim 12, wherein at least seven genes are selected from genes having Genbank accession numbers M68891, X51757, D83004, X06256, Z22865, X75918 and M55153.

15. The method according to claim 12, wherein the sample is a sample of vascular tissue.

16. The method according to claim 12, wherein the vascular tissue is aortic tissue.

17. The method of claim 12, wherein at least one metagene is Metagene n, wherein n is an integer between 1 and 509.

18. The method of claim 12, wherein the one or more predictive statistical tree models correctly classify samples with greater than 85% accuracy.

19. The method of claim 12, wherein the one or more predictive statistical tree models correctly classify samples with greater than 90% accuracy.
